SEQUENCE LISTING (1049 sequences)

SEQ ID NO: 1 - protein, 220 aa, Homo sapiens
  source 1..220: note = sp|P10747|CD28_HUMAN T-cell-specific surface glycoprotein CD28 OS=Homo sapiens OX=9606 GN=CD28 PE=1; SV = 1
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 134 aa, Homo sapiens
  source 1..134: note = sp|P10747|19-152
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 3 - protein, 218 aa, Mus musculus
  source 1..218: note = sp|P31041|CD28_MOUSE T-cell-specific surface glycoprotein CD28 OS=Mus musculus OX=10090 GN=Cd28 PE=1; SV = 2
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 4 - protein, 131 aa, Mus musculus
  source 1..131: note = sp|P31041|20-150
NKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKL

SEQ ID NO: 5 - protein, 220 aa, Macaca fascicularis
  source 1..220: note = tr|Q0PDN3|Q0PDN3_MACFA CD28 OS=Macaca fascicularis; OX = 9541 GN=CD28 PE=2 SV=1
MLRLLLALNLLPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWALVVVGGVLACYSLLVTVAFCIFWMRSKRSRLLHSDYMNMTPRRPGPTRKHYQPYAPPRDFAAYRS

SEQ ID NO: 6 - protein, 134 aa, Macaca fascicularis; identical to SEQ ID NO: 2
  source 1..134: note = tr|Q0PDN3|19-152

SEQ ID NO: 7 - protein, 584 aa, Macaca fascicularis
  source 1..584: note = XP_015308533.1 PREDICTED: CD276 antigen isoform X1 [Macaca fascicularis]
MKLSSDHVFPLFRKLQWLPAAFRIQFTPVSPSAGAAFHHGEPSCQLPHSKMLHRRGSPGMGVHVGAALGALWFCLTGALEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEALWVTVGLSVCLVALLVALAFVCWRKIKQSCEEENAGAEDQDGEGEGSKTALQPLKHSDSKEDDGQELA

SEQ ID NO: 8 - protein, 438 aa, Macaca fascicularis
  source 1..438: note = XP_015308533.1|79-516
LEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEA

SEQ ID NO: 9 - protein, 121 aa, synthetic construct
  source 1..121: note = 1A7[CD28]_H1L1 Variable heavy (vh) domain
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 10 - protein, 5 aa, synthetic construct
  source 1..5: note = 1A7[CD28]_H1L1 vhCDR1
SYAMS

SEQ ID NO: 11 - protein, 17 aa, synthetic construct
  source 1..17: note = 1A7[CD28]_H1L1 vhCDR2
TISGSGDSTYYADSVKG

SEQ ID NO: 12 - protein, 12 aa, synthetic construct
  source 1..12: note = 1A7[CD28]_H1L1 vhCDR3
SGPGLRQVGFDY

SEQ ID NO: 13 - protein, 107 aa, synthetic construct
  source 1..107: note = 1A7[CD28]_H1L1 Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 14 - protein, 11 aa, synthetic construct
  source 1..11: note = 1A7[CD28]_H1L1 vlCDR1
RASQSISSYLN

SEQ ID NO: 15 - protein, 7 aa, synthetic construct
  source 1..7: note = 1A7[CD28]_H1L1 vlCDR2
AASSLQS

SEQ ID NO: 16 - protein, 9 aa, synthetic construct
  source 1..9: note = 1A7[CD28]_H1L1 vlCDR3
QQSYSTPFT

SEQ ID NO: 17 - protein, 450 aa, synthetic construct
  source 1..450: note = XENP28428 Heavy Chain
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, synthetic construct
  source 1..214: note = XENP28428 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 121 aa, synthetic construct
  source 1..121: note = 1A7_H1.1 Variable heavy (vh) domain
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 20 - protein, 5 aa, synthetic construct
  source 1..5: note = 1A7_H1.1 vhCDR1
SYYMS

SEQ ID NO: 21 - protein, 17 aa, synthetic construct
  source 1..17: note = 1A7_H1.1 vhCDR2
TISGSGDSTYYADSVKG

SEQ ID NO: 22 - protein, 12 aa, synthetic construct
  source 1..12: note = 1A7_H1.1 vhCDR3
SGPGLRQVGFDY

SEQ ID NO: 23 - protein, 121 aa, synthetic construct
  source 1..121: note = 1A7_H1.14 Variable heavy (vh) domain
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 24 - protein, 5 aa, synthetic construct
  source 1..5: note = 1A7_H1.14 vhCDR1
SYYMS

SEQ ID NO: 25 - protein, 17 aa, synthetic construct
  source 1..17: note = 1A7_H1.14 vhCDR2
TISESGDSTYYADSVKG

SEQ ID NO: 26 - protein, 12 aa, synthetic construct
  source 1..12: note = 1A7_H1.14 vhCDR3
SGPGLRQVGFDY

SEQ ID NO: 27 - protein, 107 aa, synthetic construct
  source 1..107: note = 1A7_L1.71 Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 28 - protein, 11 aa, synthetic construct
  source 1..11: note = 1A7_L1.71 vlCDR1
RASQSISSYLN

SEQ ID NO: 29 - protein, 7 aa, synthetic construct
  source 1..7: note = 1A7_L1.71 vlCDR2
AASSLQS

SEQ ID NO: 30 - protein, 9 aa, synthetic construct
  source 1..9: note = 1A7_L1.71 vlCDR3
QQVYSTPFT

SEQ ID NO: 31 - protein, 121 aa, synthetic construct; identical to SEQ ID NO: 19
  source 1..121: note = 1A7[CD28]_H1.1_L1 Variable Heavy (vh) Domain

SEQ ID NO: 32 - protein, 5 aa, synthetic construct
  source 1..5: note = 1A7[CD28]_H1.1_L1 vhCDR1
SYYMS

SEQ ID NO: 33 - protein, 17 aa, synthetic construct
  source 1..17: note = 1A7[CD28]_H1.1_L1 vhCDR2
TISGSGDSTYYADSVKG

SEQ ID NO: 34 - protein, 12 aa, synthetic construct
  source 1..12: note = 1A7[CD28]_H1.1_L1 vhCDR3
SGPGLRQVGFDY

SEQ ID NO: 35 - protein, 107 aa, synthetic construct; identical to SEQ ID NO: 13
  source 1..107: note = 1A7[CD28]_H1.1_L1 Variable Light (vl) Domain

SEQ ID NO: 36 - protein, 11 aa, synthetic construct
  source 1..11: note = 1A7[CD28]_H1.1_L1 vlCDR1
RASQSISSYLN

SEQ ID NO: 37 - protein, 7 aa, synthetic construct
  source 1..7: note = 1A7[CD28]_H1.1_L1 vlCDR2
AASSLQS

SEQ ID NO: 38 - protein, 9 aa, synthetic construct
  source 1..9: note = 1A7[CD28]_H1.1_L1 vlCDR3
QQSYSTPFT

SEQ ID NO: 39 - protein, 121 aa, synthetic construct; identical to SEQ ID NO: 9
  source 1..121: note = 1A7[CD28]_H1_L1.71 Variable Heavy (vh) Domain

SEQ ID NO: 40 - protein, 5 aa, synthetic construct
  source 1..5: note = 1A7[CD28]_H1_L1.71 vhCDR1
SYAMS

SEQ ID NO: 41 - protein, 17 aa, synthetic construct
  source 1..17: note = 1A7[CD28]_H1_L1.71 vhCDR2
TISGSGDSTYYADSVKG

SEQ ID NO: 42 - protein, 12 aa, synthetic construct
  source 1..12: note = 1A7[CD28]_H1_L1.71 vhCDR3
SGPGLRQVGFDY

SEQ ID NO: 43 - protein, 107 aa, synthetic construct; identical to SEQ ID NO: 27
  source 1..107: note = 1A7[CD28]_H1_L1.71 Variable Light (vl) Domain

SEQ ID NO: 44 - protein, 11 aa, synthetic construct
  source 1..11: note = 1A7[CD28]_H1_L1.71 vlCDR1
RASQSISSYLN

SEQ ID NO: 45 - protein, 7 aa, synthetic construct
  source 1..7: note = 1A7[CD28]_H1_L1.71 vlCDR2
AASSLQS

SEQ ID NO: 46 - protein, 9 aa, synthetic construct
  source 1..9: note = 1A7[CD28]_H1_L1.71 vlCDR3
QQVYSTPFT

SEQ ID NO: 47 - protein, 121 aa, synthetic construct; identical to SEQ ID NO: 19
  source 1..121: note = 1A7[CD28]_H1.1_L1.71 Variable Heavy (vh) Domain

SEQ ID NO: 48 - protein, 5 aa, synthetic construct
  source 1..5: note = 1A7[CD28]_H1.1_L1.71 vhCDR1
SYYMS

SEQ ID NO: 49 - protein, 17 aa, synthetic construct
  source 1..17: note = 1A7[CD28]_H1.1_L1.71 vhCDR2
TISGSGDSTYYADSVKG

SEQ ID NO: 50 - protein, 12 aa, synthetic construct
  source 1..12: note = 1A7[CD28]_H1.1_L1.71 vhCDR3
SGPGLRQVGFDY

SEQ ID NO: 51 - protein, 107 aa, synthetic construct; identical to SEQ ID NO: 27
  source 1..107: note = 1A7[CD28]_H1.1_L1.71 Variable Light (vl) Domain

SEQ ID NO: 52 - protein, 11 aa, synthetic construct
  source 1..11: note = 1A7[CD28]_H1.1_L1.71 vlCDR1
RASQSISSYLN

SEQ ID NO: 53 - protein, 7 aa, synthetic construct
  source 1..7: note = 1A7[CD28]_H1.1_L1.71 vlCDR2
AASSLQS

SEQ ID NO: 54 - protein, 9 aa, synthetic construct
  source 1..9: note = 1A7[CD28]_H1.1_L1.71 vlCDR3
QQVYSTPFT

SEQ ID NO: 55 - protein, 121 aa, synthetic construct; identical to SEQ ID NO: 23
  source 1..121: note = 1A7[CD28]_H1.14_L1 Variable Heavy (vh) Domain

SEQ ID NO: 56 - protein, 5 aa, synthetic construct
  source 1..5: note = 1A7[CD28]_H1.14_L1 vhCDR1
SYYMS

SEQ ID NO: 57 - protein, 17 aa, synthetic construct
  source 1..17: note = 1A7[CD28]_H1.14_L1 vhCDR2
TISESGDSTYYADSVKG

SEQ ID NO: 58 - protein, 12 aa, synthetic construct
  source 1..12: note = 1A7[CD28]_H1.14_L1 vhCDR3
SGPGLRQVGFDY

SEQ ID NO: 59 - protein, 107 aa, synthetic construct; identical to SEQ ID NO: 13
  source 1..107: note = 1A7[CD28]_H1.14_L1 Variable Light (vl) Domain

SEQ ID NO: 60 - protein, 11 aa, synthetic construct
  source 1..11: note = 1A7[CD28]_H1.14_L1 vlCDR1
RASQSISSYLN

SEQ ID NO: 61 - protein, 7 aa, synthetic construct
  source 1..7: note = 1A7[CD28]_H1.14_L1 vlCDR2
AASSLQS

SEQ ID NO: 62 - protein, 9 aa, synthetic construct
  source 1..9: note = 1A7[CD28]_H1.14_L1 vlCDR3
QQSYSTPFT

SEQ ID NO: 63 - protein, 121 aa, synthetic construct; identical to SEQ ID NO: 23
  source 1..121: note = 1A7[CD28]_H1.14_L1.71 Variable Heavy (vh) Domain

SEQ ID NO: 64 - protein, 5 aa, synthetic construct
  source 1..5: note = 1A7[CD28]_H1.14_L1.71 vhCDR1
SYYMS

SEQ ID NO: 65 - protein, 17 aa, synthetic construct
  source 1..17: note = 1A7[CD28]_H1.14_L1.71 vhCDR2
TISESGDSTYYADSVKG

SEQ ID NO: 66 - protein, 12 aa, synthetic construct
  source 1..12: note = 1A7[CD28]_H1.14_L1.71 vhCDR3
SGPGLRQVGFDY

SEQ ID NO: 67 - protein, 107 aa, synthetic construct; identical to SEQ ID NO: 27
  source 1..107: note = 1A7[CD28]_H1.14_L1.71 Variable Light (vl) Domain

SEQ ID NO: 68 - protein, 11 aa, synthetic construct
  source 1..11: note = 1A7[CD28]_H1.14_L1.71 vlCDR1
RASQSISSYLN

SEQ ID NO: 69 - protein, 7 aa, synthetic construct
  source 1..7: note = 1A7[CD28]_H1.14_L1.71 vlCDR2
AASSLQS

SEQ ID NO: 70 - protein, 9 aa, synthetic construct
  source 1..9: note = 1A7[CD28]_H1.14_L1.71 vlCDR3
QQVYSTPFT

SEQ ID NO: 71 - protein, 30 aa, synthetic construct
  source 1..30: note = 1A7 VH HFR1
  VARIANT 28: note = T, S, or N
  VARIANT 29: note = F or L
  VARIANT 30: note = S, E, R, K, G, T, A, or N
EVQLLESGGGLVQPGGSLRLSCAASGFXXX

SEQ ID NO: 72 - skipped

SEQ ID NO: 73 - protein, 14 aa, synthetic construct
  source 1..14: note = 1A7 VH HFR2
  VARIANT 14: note = S or A
WVRQAPGKGLEWVX

SEQ ID NO: 74 - protein, 17 aa, synthetic construct
  source 1..17: note = 1A7 VH HCDR2
  VARIANT 1: note = T or S
  VARIANT 3: note = S, D, E, Y, or T
  VARIANT 4: note = G, D, E, A, Y, S, N, or T
  VARIANT 5: note = S, D, N, or G
  VARIANT 6: note = D, T, Y, S, or A
  VARIANT 7: note = D, T, Y, S, or A
  VARIANT 8: note = S, Y, A, T, D, or N
XIXXXXXXTYYADSVKG

SEQ ID NO: 75 - protein, 32 aa, synthetic construct
  source 1..32: note = 1A7 VH HFR3
RFTISRDNSKNTLYLQMNSLRAEDTAVYYCAK

SEQ ID NO: 76 - protein, 12 aa, synthetic construct
  source 1..12: note = 1A7 VH HCDR3
SGPGLRQVGFDY

SEQ ID NO: 77 - protein, 11 aa, synthetic construct
  source 1..11: note = 1A7 VH HFR4
WGQGTLVTVSS

SEQ ID NO: 78 - skipped

SEQ ID NO: 79 - protein, 23 aa, synthetic construct
  source 1..23: note = 1A7 VL LFR1
DIQMTQSPSSLSASVGDRVTITC

SEQ ID NO: 80 - protein, 11 aa, synthetic construct
  source 1..11: note = 1A7 VL LCDR1
  VARIANT 8: note = S, A, D, G, H, K, N, Q, T, V, or Y
  VARIANT 9: note = Y, A, D, F, H, K, L, N, Q, S, or W
  VARIANT 11: note = N, A, D, G, H, Q, S, T, or Y
RASQSISXXLX

SEQ ID NO: 81 - protein, 15 aa, synthetic construct
  source 1..15: note = 1A7 VL LFR2
WYQQKPGKAPKLLIY

SEQ ID NO: 82 - skipped

SEQ ID NO: 83 - protein, 32 aa, synthetic construct
  source 1..32: note = 1A7 VL LFR3
GVPSRFSGSGSGTDFTLTISSLQPEDFATYYC

SEQ ID NO: 84 - protein, 9 aa, synthetic construct
  source 1..9: note = 1A7 VL LCDR3
  VARIANT 3: note = S, A, D, F, H, K, L, T, V, or Y
  VARIANT 4: note = Y, A, D, F, H, K, L, Q, V, or W
  VARIANT 5: note = S, A, D, G, H, K, N, Q, T, V, or Y
  VARIANT 6: note = T, A, D, F, I, K, L, Q, S, V, or Y
  VARIANT 8: note = F, I, L, or W
QQXXXXPXT

SEQ ID NO: 85 - protein, 10 aa, synthetic construct
  source 1..10: note = 1A7 VL LFR4
FGQGTKLEIK

SEQ ID NO: 86 - protein, 121 aa, synthetic construct
  source 1..121: note = CD28.3[CD28]_H0L0 Variable heavy (vh) domain
EVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSS

SEQ ID NO: 87 - protein, 5 aa, synthetic construct
  source 1..5: note = CD28.3[CD28]_H0L0 vhCDR1
EYIIH

SEQ ID NO: 88 - protein, 17 aa, synthetic construct
  source 1..17: note = CD28.3[CD28]_H0L0 vhCDR2
WFYPGSNDIQYNAKFKG

SEQ ID NO: 89 - protein, 12 aa, synthetic construct
  source 1..12: note = CD28.3[CD28]_H0L0 vhCDR3
RDDFSGYDALPY

SEQ ID NO: 90 - protein, 107 aa, synthetic construct
  source 1..107: note = CD28.3[CD28]_H0L0 Variable light (vl) domain
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIK

SEQ ID NO: 91 - protein, 11 aa, synthetic construct
  source 1..11: note = CD28.3[CD28]_H0L0 vlCDR1
RTNENIYSNLA

SEQ ID NO: 92 - protein, 7 aa, synthetic construct
  source 1..7: note = CD28.3[CD28]_H0L0 vlCDR2
AATHLVE

SEQ ID NO: 93 - protein, 9 aa, synthetic construct
  source 1..9: note = CD28.3[CD28]_H0L0 vlCDR3
QHFWGTPCT

SEQ ID NO: 94 - protein, 121 aa, synthetic construct
  source 1..121: note = hCD28.3[CD28]_H1L1 Variable heavy (vh) domain
QVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 95 - protein, 5 aa, synthetic construct
  source 1..5: note = hCD28.3[CD28]_H1L1 vhCDR1
EYIIH

SEQ ID NO: 96 - protein, 17 aa, synthetic construct
  source 1..17: note = hCD28.3[CD28]_H1L1 vhCDR2
WFYPGSNDIQYNAQFKG

SEQ ID NO: 97 - protein, 12 aa, synthetic construct
  source 1..12: note = hCD28.3[CD28]_H1L1 vhCDR3
RDDFSGYDALPY

SEQ ID NO: 98 - protein, 107 aa, synthetic construct
  source 1..107: note = hCD28.3[CD28]_H1L1 Variable light (vl) domain
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIK

SEQ ID NO: 99 - protein, 11 aa, synthetic construct
  source 1..11: note = hCD28.3[CD28]_H1L1 vlCDR1
KTNENIYSNLA

SEQ ID NO: 100 - protein, 7 aa, synthetic construct
  source 1..7: note = hCD28.3[CD28]_H1L1 vlCDR2
AATHLVE

SEQ ID NO: 101 - protein, 9 aa, synthetic construct
  source 1..9: note = hCD28.3[CD28]_H1L1 vlCDR3
QHFWGTPCT

SEQ ID NO: 102 - protein, 120 aa, synthetic construct
  source 1..120: note = 5.11A1[CD28]_H0L0 Variable heavy (vh) domain
QVQLQQSGPELVKPGTSVRISCEASGYTFTSYYIHWVKQRPGQGLEWIGCIYPGNVNTNYNEKFKDKATLIVDTSSNTAYMQLSRMTSEDSAVYFCTRSHYGLDWNFDVWGAGTTVTVSS

SEQ ID NO: 103 - protein, 5 aa, synthetic construct
  source 1..5: note = 5.11A1[CD28]_H0L0 vhCDR1
SYYIH

SEQ ID NO: 104 - protein, 17 aa, synthetic construct
  source 1..17: note = 5.11A1[CD28]_H0L0 vhCDR2
CIYPGNVNTNYNEKFKD

SEQ ID NO: 105 - protein, 11 aa, synthetic construct
  source 1..11: note = 5.11A1[CD28]_H0L0 vhCDR3
SHYGLDWNFDV

SEQ ID NO: 106 - protein, 107 aa, synthetic construct
  source 1..107: note = 5.11A1[CD28]_H0L0 Variable light (vl) domain
DIQMNQSPSSLSASLGDTITITCHASQNIYVWLNWYQQKPGNIPKLLIYKASNLHTGVPSRFSGSGSGTGFTLTISSLQPEDIATYYCQQGQTYPYTFGGGTKLEIK

SEQ ID NO: 107 - protein, 11 aa, synthetic construct
  source 1..11: note = 5.11A1[CD28]_H0L0 vlCDR1
HASQNIYVWLN

SEQ ID NO: 108 - protein, 7 aa, synthetic construct
  source 1..7: note = 5.11A1[CD28]_H0L0 vlCDR2
KASNLHT

SEQ ID NO: 109 - protein, 9 aa, synthetic construct
  source 1..9: note = 5.11A1[CD28]_H0L0 vlCDR3
QQGQTYPYT

SEQ ID NO: 110 - protein, 120 aa, synthetic construct
  source 1..120: note = TGN1412_H1L1 Variable heavy (vh) domain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 111 - protein, 5 aa, synthetic construct
  source 1..5: note = TGN1412_H1L1 vhCDR1
SYYIH

SEQ ID NO: 112 - protein, 17 aa, synthetic construct
  source 1..17: note = TGN1412_H1L1 vhCDR2
CIYPGNVNTNYNEKFKD

SEQ ID NO: 113 - protein, 11 aa, synthetic construct
  source 1..11: note = TGN1412_H1L1 vhCDR3
SHYGLDWNFDV

SEQ ID NO: 114 - protein, 107 aa, synthetic construct
  source 1..107: note = TGN1412_H1L1 Variable light (vl) domain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 115 - protein, 11 aa, synthetic construct
  source 1..11: note = TGN1412_H1L1 vlCDR1
HASQNIYVWLN

SEQ ID NO: 116 - protein, 7 aa, synthetic construct
  source 1..7: note = TGN1412_H1L1 vlCDR2
KASNLHT

SEQ ID NO: 117 - protein, 9 aa, synthetic construct
  source 1..9: note = TGN1412_H1L1 vlCDR3
QQGQTYPYT

SEQ ID NO: 118 - protein, 127 aa, synthetic construct
  source 1..127: note = 341VL34[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLTTEDTAVYYCNTDLPYYYGSGRYSGMDVWGQGTTVTVSS

SEQ ID NO: 119 - protein, 5 aa, synthetic construct
  source 1..5: note = 341VL34[CD28]_H1L1 vhCDR1
NAWMN

SEQ ID NO: 120 - protein, 19 aa, synthetic construct
  source 1..19: note = 341VL34[CD28]_H1L1 vhCDR2
RIKGKTDGGTADYAAPVKG

SEQ ID NO: 121 - protein, 16 aa, synthetic construct
  source 1..16: note = 341VL34[CD28]_H1L1 vhCDR3
DLPYYYGSGRYSGMDV

SEQ ID NO: 122 - protein, 104 aa, synthetic construct
  source 1..104: note = 341VL34[CD28]_H1L1 Variable light (vl) domain
EIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIK

SEQ ID NO: 123 - protein, 11 aa, synthetic construct
  source 1..11: note = 341VL34[CD28]_H1L1 vlCDR1
RASQSVSSYLA

SEQ ID NO: 124 - protein, 7 aa, synthetic construct
  source 1..7: note = 341VL34[CD28]_H1L1 vlCDR2
DASNRAT

SEQ ID NO: 125 - protein, 6 aa, synthetic construct
  source 1..6: note = 341VL34[CD28]_H1L1 vlCDR3
QQRSNT

SEQ ID NO: 126 - protein, 127 aa, synthetic construct
  source 1..127: note = 341VL36[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLKTEDTGVYYCTTYLPYYYGSERWSGMDVWGQGTTVTVSS

SEQ ID NO: 127 - protein, 5 aa, synthetic construct
  source 1..5: note = 341VL36[CD28]_H1L1 vhCDR1
NAWMN

SEQ ID NO: 128 - protein, 19 aa, synthetic construct
  source 1..19: note = 341VL36[CD28]_H1L1 vhCDR2
RIKGKTDGGTADYAAPVKG

SEQ ID NO: 129 - protein, 16 aa, synthetic construct
  source 1..16: note = 341VL36[CD28]_H1L1 vhCDR3
YLPYYYGSERWSGMDV

SEQ ID NO: 130 - protein, 104 aa, synthetic construct; identical to SEQ ID NO: 122
  source 1..104: note = 341VL36[CD28]_H1L1 Variable light (vl) domain

SEQ ID NO: 131 - protein, 11 aa, synthetic construct
  source 1..11: note = 341VL36[CD28]_H1L1 vlCDR1
RASQSVSSYLA

SEQ ID NO: 132 - protein, 7 aa, synthetic construct
  source 1..7: note = 341VL36[CD28]_H1L1 vlCDR2
DASNRAT

SEQ ID NO: 133 - protein, 6 aa, synthetic construct
  source 1..6: note = 341VL36[CD28]_H1L1 vlCDR3
QQRSNT

SEQ ID NO: 134 - protein, 126 aa, synthetic construct
  source 1..126: note = 281VL4[CD28]_H1L1 Variable heavy (vh) domain
EVQLVQSGSELKKPGSSVKVSCKASGGTSRSFAISWVRQAPGQGLEWMGGIIPIFGPANYAQKFQGRVTITADESTSTAYMELSSLRSEDTAVYYCARHAIAMGWGVITTNYFDSWGQGTMVTVSS

SEQ ID NO: 135 - protein, 5 aa, synthetic construct
  source 1..5: note = 281VL4[CD28]_H1L1 vhCDR1
SFAIS

SEQ ID NO: 136 - protein, 17 aa, synthetic construct
  source 1..17: note = 281VL4[CD28]_H1L1 vhCDR2
GIIPIFGPANYAQKFQG

SEQ ID NO: 137 - protein, 17 aa, synthetic construct
  source 1..17: note = 281VL4[CD28]_H1L1 vhCDR3
HAIAMGWGVITTNYFDS

SEQ ID NO: 138 - protein, 108 aa, synthetic construct
  source 1..108: note = 281VL4[CD28]_H1L1 Variable light (vl) domain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIK

SEQ ID NO: 139 - protein, 12 aa, synthetic construct
  source 1..12: note = 281VL4[CD28]_H1L1 vlCDR1
RASQSVSSSYLA

SEQ ID NO: 140 - protein, 7 aa, synthetic construct
  source 1..7: note = 281VL4[CD28]_H1L1 vlCDR2
GASSRAT

SEQ ID NO: 141 - protein, 9 aa, synthetic construct
  source 1..9: note = 281VL4[CD28]_H1L1 vlCDR3
QQYGSSPIT

SEQ ID NO: 142 - protein, 121 aa, synthetic construct
  source 1..121: note = HuTN228[CD28]_H1L1 Variable heavy (vh) domain
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 143 - protein, 5 aa, synthetic construct
  source 1..5: note = HuTN228[CD28]_H1L1 vhCDR1
SYGVH

SEQ ID NO: 144 - protein, 16 aa, synthetic construct
  source 1..16: note = HuTN228[CD28]_H1L1 vhCDR2
VIWPGGGTNFNSALMS

SEQ ID NO: 145 - protein, 13 aa, synthetic construct
  source 1..13: note = HuTN228[CD28]_H1L1 vhCDR3
DRAYGNYLYAMDY

SEQ ID NO: 146 - protein, 111 aa, synthetic construct
  source 1..111: note = HuTN228[CD28]_H1L1 Variable light (vl) domain
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 147 - protein, 15 aa, synthetic construct
  source 1..15: note = HuTN228[CD28]_H1L1 vlCDR1
RASESVEYYVTSLMQ

SEQ ID NO: 148 - protein, 7 aa, synthetic construct
  source 1..7: note = HuTN228[CD28]_H1L1 vlCDR2
AASNVDS

SEQ ID NO: 149 - protein, 9 aa, synthetic construct
  source 1..9: note = HuTN228[CD28]_H1L1 vlCDR3
QQSRKVPFT

SEQ ID NO: 150 - protein, 124 aa, synthetic construct
  source 1..124: note = PV1[CD28]_H0L0 Variable heavy (vh) domain
QVQLKQSGAELVKPGASVKISCKTSGYTFTDGYMNWVEQKPGQGLEWIGRIDPDSGNTRYNQKFQGKATLTRDKSSSTVYMDLRSLTSEDSAVYYCARDGTFYGTYGYWYFDFWGQGTQVTVSS

SEQ ID NO: 151 - protein, 5 aa, synthetic construct
  source 1..5: note = PV1[CD28]_H0L0 vhCDR1
DGYMN

SEQ ID NO: 152 - protein, 17 aa, synthetic construct
  source 1..17: note = PV1[CD28]_H0L0 vhCDR2
RIDPDSGNTRYNQKFQG

SEQ ID NO: 153 - protein, 15 aa, synthetic construct
  source 1..15: note = PV1[CD28]_H0L0 vhCDR3
DGTFYGTYGYWYFDF

SEQ ID NO: 154 - protein, 113 aa, synthetic construct
  source 1..113: note = PV1[CD28]_H0L0 Variable light (vl) domain
DIVMTQSPYSLAVSAGEKVTMSCRSSQSLYYSGIKKNLLAWYQQKPGQSPKLLIYFTSTRLPGVPDRFTGSGSGTDYTLTITSVQAEDMGHYFCQQGISTPLTFGDGTKLEIR

SEQ ID NO: 155 - protein, 17 aa, synthetic construct
  source 1..17: note = PV1[CD28]_H0L0 vlCDR1
RSSQSLYYSGIKKNLLA

SEQ ID NO: 156 - protein, 7 aa, synthetic construct
  source 1..7: note = PV1[CD28]_H0L0 vlCDR2
FTSTRLP

SEQ ID NO: 157 - protein, 9 aa, synthetic construct
  source 1..9: note = PV1[CD28]_H0L0 vlCDR3
QQGISTPLT

SEQ ID NO: 158 - protein, 120 aa, synthetic construct
  source 1..120: note = m9.3[CD28]_H0L0 Variable heavy (vh) domain
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 159 - protein, 5 aa, synthetic construct
  source 1..5: note = m9.3[CD28]_H0L0 vhCDR1
DYGVH

SEQ ID NO: 160 - protein, 16 aa, synthetic construct
  source 1..16: note = m9.3[CD28]_H0L0 vhCDR2
VIWAGGGTNYNSALMS

SEQ ID NO: 161 - protein, 12 aa, synthetic construct
  source 1..12: note = m9.3[CD28]_H0L0 vhCDR3
DKGYSYYYSMDY

SEQ ID NO: 162 - protein, 111 aa, synthetic construct
  source 1..111: note = m9.3[CD28]_H0L0 Variable light (vl) domain
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 163 - protein, 15 aa, synthetic construct
  source 1..15: note = m9.3[CD28]_H0L0 vlCDR1
RASESVEYYVTSLMQ

SEQ ID NO: 164 - protein, 7 aa, synthetic construct
  source 1..7: note = m9.3[CD28]_H0L0 vlCDR2
AASNVES

SEQ ID NO: 165 - protein, 9 aa, synthetic construct
  source 1..9: note = m9.3[CD28]_H0L0 vlCDR3
QQSRKVPYT

SEQ ID NO: 166 - protein, 120 aa, synthetic construct
  source 1..120: note = hu9.3[CD28]_H1L1 Variable heavy (vh) domain
EVQLVQSGGGLVQPGGSLRLSCAGSGFTFSDYGVHWVRQAPGKGLEWVSAIWAGGGTNYASSVMGRFTISRDNAKNSLYLQMNSLRAEDMAVYYCARDKGYSYYYSMDYWGQGTLVTVSS

SEQ ID NO: 167 - protein, 5 aa, synthetic construct
  source 1..5: note = hu9.3[CD28]_H1L1 vhCDR1
DYGVH

SEQ ID NO: 168 - protein, 16 aa, synthetic construct
  source 1..16: note = hu9.3[CD28]_H1L1 vhCDR2
AIWAGGGTNYASSVMG

SEQ ID NO: 169 - protein, 12 aa, synthetic construct
  source 1..12: note = hu9.3[CD28]_H1L1 vhCDR3
DKGYSYYYSMDY

SEQ ID NO: 170 - protein, 111 aa, synthetic construct
  source 1..111: note = hu9.3[CD28]_H1L1 Variable light (vl) domain
DIVMTQSPDSLAVSLGERATINCRASESVEYYVTSLMAWYQQKPGQPPKLLIYAASNVESGVPDRFSGSGSGTNFSLTISSLQAEDVAVYYCQQSRKVPYTFGQGTKLEIK

SEQ ID NO: 171 - protein, 15 aa, synthetic construct
  source 1..15: note = hu9.3[CD28]_H1L1 vlCDR1
RASESVEYYVTSLMA

SEQ ID NO: 172 - protein, 7 aa, synthetic construct
  source 1..7: note = hu9.3[CD28]_H1L1 vlCDR2
AASNVES

SEQ ID NO: 173 - protein, 9 aa, synthetic construct
  source 1..9: note = hu9.3[CD28]_H1L1 vlCDR3
QQSRKVPYT

SEQ ID NO: 174 - protein, 127 aa, synthetic construct
  source 1..127: note = 9G2[CD28]_H0L0 Variable heavy (vh) domain
EVQLVESGGGLVQPGRSLKLSCAASGFTFSNYYMAWVRQAPTKGLEWVASITNTGGSTYYRDSVKGRFTISRDNAKSTLYLQMDSLRSEDTATYYCTRGLIYYYDGRNYYDYVMDAWGQGASVTVSS

SEQ ID NO: 175 - protein, 5 aa, synthetic construct
  source 1..5: note = 9G2[CD28]_H0L0 vhCDR1
NYYMA

SEQ ID NO: 176 - protein, 17 aa, synthetic construct
  source 1..17: note = 9G2[CD28]_H0L0 vhCDR2
SITNTGGSTYYRDSVKG

SEQ ID NO: 177 - protein, 18 aa, synthetic construct
  source 1..18: note = 9G2[CD28]_H0L0 vhCDR3
GLIYYYDGRNYYDYVMDA

SEQ ID NO: 178 - protein, 107 aa, synthetic construct
  source 1..107: note = 9G2[CD28]_H0L0 Variable light (vl) domain
DIQMTQSPASLSASLGETVSIECLASEGISNSLAWYQQKPGKSPQLLIYGASSLQDGVPSRFSGSGSGTQYSLKISGMQPEDEGVYYCQQGYKYPLTFGSGTKLEIK

SEQ ID NO: 179 - protein, 11 aa, synthetic construct
  source 1..11: note = 9G2[CD28]_H0L0 vlCDR1
LASEGISNSLA

SEQ ID NO: 180 - protein, 7 aa, synthetic construct
  source 1..7: note = 9G2[CD28]_H0L0 vlCDR2
GASSLQD

SEQ ID NO: 181 - protein, 9 aa, synthetic construct
  source 1..9: note = 9G2[CD28]_H0L0 vlCDR3
QQGYKYPLT

SEQ ID NO: 182 - protein, 127 aa, synthetic construct
  source 1..127: note = 9G2[CD28]_H1L1 Variable heavy (vh) domain
EVQLLESGGGLVQPGGSLRLSCAASGFTFSNYYMAWVRQAPGKGLEWVSSITNTGGSTYYRDSVKGRFTISRDNSKSTLYLQMNSLRAEDTAVYYCTRGLIYYYDGRNYYDYVMDAWGQGTTVTVSS

SEQ ID NO: 183 - protein, 5 aa, synthetic construct
  source 1..5: note = 9G2[CD28]_H1L1 vhCDR1
NYYMA

SEQ ID NO: 184 - protein, 17 aa, synthetic construct
  source 1..17: note = 9G2[CD28]_H1L1 vhCDR2
SITNTGGSTYYRDSVKG

SEQ ID NO: 185 - protein, 18 aa, synthetic construct
  source 1..18: note = 9G2[CD28]_H1L1 vhCDR3
GLIYYYDGRNYYDYVMDA

SEQ ID NO: 186 - protein, 107 aa, synthetic construct
  source 1..107: note = 9G2[CD28]_H1L1 Variable light (vl) domain
DIQMTQSPSSLSASVGDRVTITCRASEGISNSLAWYQQKPGKSPKLLIYGASSLQDGVPSRFSGSGSGTDYTLTISSLQPEDFATYYCQQGYKYPLTFGSGTKVEIK

SEQ ID NO: 187 - protein, 11 aa, synthetic construct
  source 1..11: note = 9G2[CD28]_H1L1 vlCDR1
RASEGISNSLA

SEQ ID NO: 188 - protein, 7 aa, synthetic construct
  source 1..7: note = 9G2[CD28]_H1L1 vlCDR2
GASSLQD

SEQ ID NO: 189 - protein, 9 aa, synthetic construct
  source 1..9: note = 9G2[CD28]_H1L1 vlCDR3
QQGYKYPLT

SEQ ID NO: 190 - protein, 121 aa, synthetic construct
  source 1..121: note = 2F10A3.140[CD28]_H1L1 Variable heavy (vh) domain
EVQLLESGGGLVQPGGSLRLSCAASGFSFGGNSMSWVRQAPGKGLEWVATISDNSYSTYYADSVKGRFTISRDNSKNTLYLQMNSLRAEDTAVYYCAKSGPGLRQVGFDYWGQGTLVTVSS

SEQ ID NO: 191 - protein, 5 aa, synthetic construct
  source 1..5: note = 2F10A3.140[CD28]_H1L1 vhCDR1
GNSMS

SEQ ID NO: 192 - protein, 17 aa, synthetic construct
  source 1..17: note = 2F10A3.140[CD28]_H1L1 vhCDR2
TISDNSYSTYYADSVKG

SEQ ID NO: 193 - protein, 12 aa, synthetic construct
  source 1..12: note = 2F10A3.140[CD28]_H1L1 vhCDR3
SGPGLRQVGFDY

SEQ ID NO: 194 - protein, 107 aa, synthetic construct; identical to SEQ ID NO: 13
  source 1..107: note = 2F10A3.140[CD28]_H1L1 Variable light (vl) domain

SEQ ID NO: 195 - protein, 11 aa, synthetic construct
  source 1..11: note = 2F10A3.140[CD28]_H1L1 vlCDR1
RASQSISSYLN

SEQ ID NO: 196 - protein, 7 aa, synthetic construct
  source 1..7: note = 2F10A3.140[CD28]_H1L1 vlCDR2
AASSLQS

SEQ ID NO: 197 - protein, 9 aa, synthetic construct
  source 1..9: note = 2F10A3.140[CD28]_H1L1 vlCDR3
QQSYSTPFT

SEQ ID NO: 198 - protein, 121 aa, synthetic construct
  source 1..121: note = TN228[CD28]_H4L2 Variable heavy (vh) domain
QVQLQESGPGLVKPSETLSLTCTVSGFSLTSYGVHWVRQPPGKGLEWIGVIWPGGGTNYNSALKSRVTISEDTSKSQVSLKLSSVTAADTAVYYCARDRAYGNYLYAMDYWGQGTLVTVSS

SEQ ID NO: 199 - protein, 5 aa, synthetic construct
  source 1..5: note = TN228[CD28]_H4L2 vhCDR1
SYGVH

SEQ ID NO: 200 - protein, 16 aa, synthetic construct
  source 1..16: note = TN228[CD28]_H4L2 vhCDR2
VIWPGGGTNYNSALKS

SEQ ID NO: 201 - protein, 13 aa, synthetic construct
  source 1..13: note = TN228[CD28]_H4L2 vhCDR3
DRAYGNYLYAMDY

SEQ ID NO: 202 - protein, 111 aa, synthetic construct
  source 1..111: note = TN228[CD28]_H4L2 Variable light (vl) domain
EIVLTQSPATLSLSPGERATLSCRASESVEYYVTSLMQWYQQKPGQAPRLLIYAASNVDSGVPARFSGSGSGTDFTLTISSLEPEDFAVYYCQQSRKVPFTFGGGTKVEIK

SEQ ID NO: 203 - protein, 15 aa, synthetic construct
  source 1..15: note = TN228[CD28]_H4L2 vlCDR1
RASESVEYYVTSLMQ

SEQ ID NO: 204 - protein, 7 aa, synthetic construct
  source 1..7: note = TN228[CD28]_H4L2 vlCDR2
AASNVDS

SEQ ID NO: 205 - protein, 9 aa, synthetic construct
  source 1..9: note = TN228[CD28]_H4L2 vlCDR3
QQSRKVPFT

SEQ ID NO: 206 - protein, 120 aa, synthetic construct
  source 1..120: note = A10[PSMA] Variable Heavy (vh) Domain
QVQLVESGGGVVQPGRSLRLSCAASGFTFSSYNMNWVRQAPGKGLEWVAIIYYDGSNKYYADSVKGRFTISRDISKNTLYLQMNSLRAEDTAVYYCARERGRDYYGMDVWGQGTTVTVSS

SEQ ID NO: 207 - protein, 5 aa, synthetic construct
  source 1..5: note = A10[PSMA] vhCDR1
SYNMN

SEQ ID NO: 208 - protein, 17 aa, synthetic construct
  source 1..17: note = A10[PSMA] vhCDR2
IIYYDGSNKYYADSVKG

SEQ ID NO: 209 - protein, 11 aa, synthetic construct
  source 1..11: note = A10[PSMA] vhCDR3
ERGRDYYGMDV

SEQ ID NO: 210 - protein, 107 aa, synthetic construct
  source 1..107: note = A10[PSMA] Variable Light (vl) Domain
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVL

SEQ ID NO: 211 - protein, 11 aa, synthetic construct
  source 1..11: note = A10[PSMA] vlCDR1
SGDALPKQYAY

SEQ ID NO: 212 - protein, 7 aa, synthetic construct
  source 1..7: note = A10[PSMA] vlCDR2
KDSERPS

SEQ ID NO: 213 - protein, 10 aa, synthetic construct
  source 1..10: note = A10[PSMA] vlCDR3
QSADSSGTYV

SEQ ID NO: 214 - protein, 120 aa, synthetic construct
  source 1..120: note = A10v2[PSMA] Variable Heavy (vh) Domain
QVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSS

SEQ ID NO: 215 - protein, 5 aa, synthetic construct
  source 1..5: note = A10v2[PSMA] vhCDR1
SYNMN

SEQ ID NO: 216 - protein, 17 aa, synthetic construct
  source 1..17: note = A10v2[PSMA] vhCDR2
IIYYDESNKYYADSVKG

SEQ ID NO: 217 - protein, 11 aa, synthetic construct
  source 1..11: note = A10v2[PSMA] vhCDR3
ERGRDYYGMDV

SEQ ID NO: 218 - protein, 107 aa, synthetic construct; identical to SEQ ID NO: 210
  source 1..107: note = A10v2[PSMA] Variable Light (vl) Domain

SEQ ID NO: 219 - protein, 11 aa, synthetic construct
  source 1..11: note = A10v2[PSMA] vlCDR1
SGDALPKQYAY

SEQ ID NO: 220 - protein, 7 aa, synthetic construct
  source 1..7: note = A10v2[PSMA] vlCDR2
KDSERPS

SEQ ID NO: 221 - protein, 10 aa, synthetic construct
  source 1..10: note = A10v2[PSMA] vlCDR3
QSADSSGTYV

SEQ ID NO: 222 - protein, 126 aa, synthetic construct
  source 1..126: note = D01[PSMA] Variable Heavy (vh) Domain
EVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCARDGVGADYGDYYYYGMDVWGQGTTVTVSS

SEQ ID NO: 223 - protein, 5 aa, synthetic construct
  source 1..5: note = D01[PSMA] vhCDR1
NYNMN

SEQ ID NO: 224 - protein, 17 aa, synthetic construct
  source 1..17: note = D01[PSMA] vhCDR2
HISTSSSNKYYADSVKG

SEQ ID NO: 225 - protein, 17 aa, synthetic construct
  source 1..17: note = D01[PSMA] vhCDR3
DGVGADYGDYYYYGMDV

SEQ ID NO: 226 - protein, 110 aa, synthetic construct
  source 1..110: note = D01[PSMA] Variable Light (vl) Domain
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSYTYVFGTGTKLTVL

SEQ ID NO: 227 - protein, 14 aa, synthetic construct
  source 1..14: note = D01[PSMA] vlCDR1
TGTSSDVGGYNYVS

SEQ ID NO: 228 - protein, 7 aa, synthetic construct
  source 1..7: note = D01[PSMA] vlCDR2
EVSNRPS

SEQ ID NO: 229 - protein, 10 aa, synthetic construct
  source 1..10: note = D01[PSMA] vlCDR3
SSYTSSYTYV

SEQ ID NO: 230 - protein, 126 aa, synthetic construct
  source 1..126: note = D01v2[PSMA] Variable Heavy (vh) Domain
EVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCAREGVGADYGDYYYYGMDVWGQGTTVTVSS

SEQ ID NO: 231 - protein, 5 aa, synthetic construct
  source 1..5: note = D01v2[PSMA] vhCDR1
NYNMN

SEQ ID NO: 232 - protein, 17 aa, synthetic construct
  source 1..17: note = D01v2[PSMA] vhCDR2
HISTSSSNKYYADSVKG

SEQ ID NO: 233 - protein, 17 aa, synthetic construct
  source 1..17: note = D01v2[PSMA] vhCDR3
EGVGADYGDYYYYGMDV

SEQ ID NO: 234 - protein, 110 aa, synthetic construct; identical to SEQ ID NO: 226
  source 1..110: note = D01v2[PSMA] Variable Light (vl) Domain

SEQ ID NO: 235 - protein, 14 aa, synthetic construct
  source 1..14: note = D01v2[PSMA] vlCDR1
TGTSSDVGGYNYVS

SEQ ID NO: 236 - protein, 7 aa, synthetic construct
  source 1..7: note = D01v2[PSMA] vlCDR2
EVSNRPS

SEQ ID NO: 237 - protein, 10 aa, synthetic construct
  source 1..10: note = D01v2[PSMA] vlCDR3
SSYTSSYTYV

SEQ ID NO: 238 - protein, 125 aa, synthetic construct
  source 1..125: note = E07[PSMA] Variable Heavy (vh) Domain
EVQLVESGGGVVQPGRSLRLSCAASGFTFITYGMHWVRQAPGKGLEWVAVVSFDESNKYYADSVKGRFTISRDNSKNTLYLQMNSLRAEDTAVYYCARALRDGNNWDYFNGMDVWGQGTTVTVSS

SEQ ID NO: 239 - protein, 5 aa, synthetic construct
  source 1..5: note = E07[PSMA] vhCDR1
TYGMH

SEQ ID NO: 240 - protein, 17 aa, synthetic construct
  source 1..17: note = E07[PSMA] vhCDR2
VVSFDESNKYYADSVKG

SEQ ID NO: 241 - protein, 16 aa, synthetic construct
  source 1..16: note = E07[PSMA] vhCDR3
ALRDGNNWDYFNGMDV

SEQ ID NO: 242 - protein, 110 aa, synthetic construct
  source 1..110: note = E07[PSMA] Variable Light (vl) Domain
QSVLTQPPSASGTPGQGVTISCSGSSSNIGSNTVNWFQQLPGTAPKLLIYSDNQRPSGVPDRFSGSKSGTSASLAISGLQSEDEADYYCAAWDDSLNGYVFGTGTKVTVL

SEQ ID NO: 243 - protein, 13 aa, synthetic construct
  source 1..13: note = E07[PSMA] vlCDR1
SGSSSNIGSNTVN

SEQ ID NO: 244 - protein, 7 aa, synthetic construct
  source 1..7: note = E07[PSMA] vlCDR2
SDNQRPS

SEQ ID NO: 245 - protein, 11 aa, synthetic construct
  source 1..11: note = E07[PSMA] vlCDR3
AAWDDSLNGYV

SEQ ID NO: 246 - protein, 118 aa, synthetic construct
  source 1..118: note = F02[PSMA] Variable Heavy (vh) Domain
EVQLLESGPGLVKPSETLSLTCTVSGGSIISYYWSWIRQPAGKGLEWIGRIYSSGSTNYNPSLKSRVTMSVDTSKNQFSLKLSSVTAADTAVYYCAKVGVWPGAFDIWGQGTMVTVSS

SEQ ID NO: 247 - protein, 5 aa, synthetic construct
  source 1..5: note = F02[PSMA] vhCDR1
SYYWS

SEQ ID NO: 248 - protein, 16 aa, synthetic construct
  source 1..16: note = F02[PSMA] vhCDR2
RIYSSGSTNYNPSLKS

SEQ ID NO: 249 - protein, 10 aa, synthetic construct
  source 1..10: note = F02[PSMA] vhCDR3
VGVWPGAFDI

SEQ ID NO: 250 - protein, 110 aa, synthetic construct
  source 1..110: note = F02[PSMA] Variable Light (vl) Domain
QSVLTQPPSASGTPGQRVTISCSGSSSNIGSNTVNWYQQLPGTAPKLLIYSSNQRPSGVPDRFSGSKSGTSASLAISGLQSEDEADYYCAAWDDSLNGVVFGGGTKLTVL

SEQ ID NO: 251 - protein, 13 aa, synthetic construct
  source 1..13: note = F02[PSMA] vlCDR1
SGSSSNIGSNTVN

SEQ ID NO: 252 - protein, 7 aa, synthetic construct
  source 1..7: note = F02[PSMA] vlCDR2
SSNQRPS

SEQ ID NO: 253 - protein, 11 aa, synthetic construct
  source 1..11: note = F02[PSMA] vlCDR3
AAWDDSLNGVV

SEQ ID NO: 254 - protein, 122 aa, synthetic construct
  source 1..122: note = A11[PSMA] Variable Heavy (vh) Domain
QVQLQESGGDVVQPGRSLRLSCAASGFSFSGYGLHWVRQAPGRGLEWVTLISYDGSNKYYADSVKGRFTISRDNSKNTLYLQMNSLRAEDTAVYYCAKTTVSDPYYYGMDVWGQGTTVTVSS

SEQ ID NO: 255 - protein, 5 aa, synthetic construct
  source 1..5: note = A11[PSMA] vhCDR1
GYGLH

SEQ ID NO: 256 - protein, 17 aa, synthetic construct
  source 1..17: note = A11[PSMA] vhCDR2
LISYDGSNKYYADSVKG

SEQ ID NO: 257 - protein, 13 aa, synthetic construct
  source 1..13: note = A11[PSMA] vhCDR3
TTVSDPYYYGMDV

SEQ ID NO: 258 - protein, 108 aa, synthetic construct
  source 1..108: note = A11[PSMA] Variable Light (vl) Domain
SYELTQPPSVSVAPGQTARITCGGNNIGSKSVHWYQQKPGQAPVLVVYDDSDRPSGIPERFSGTNSGNTATLTISRAEAGDEADYYCQVWDSSSDHVVFGGGTKLTVL

SEQ ID NO: 259 - protein, 11 aa, synthetic construct
  source 1..11: note = A11[PSMA] vlCDR1
GGNNIGSKSVH

SEQ ID NO: 260 - protein, 7 aa, synthetic construct
  source 1..7: note = A11[PSMA] vlCDR2
DDSDRPS

SEQ ID NO: 261 - protein, 11 aa, synthetic construct
  source 1..11: note = A11[PSMA] vlCDR3
QVWDSSSDHVV

SEQ ID NO: 262 - protein, 122 aa, synthetic construct
  source 1..122: note = F07[PSMA] Variable Heavy (vh) Domain
EVQLVESGGGVVQPGRSLRLSCAASGFTFSSYGMNWVRQAPGKGLEWVAVTSYDGSNKYYADSVKGRFTISRDISKNTLYLQMSSLRAEDTAVYYCARDPYSSSWNGAFDIWGPGTMVTVSS

SEQ ID NO: 263 - protein, 5 aa, synthetic construct
  source 1..5: note = F07[PSMA] vhCDR1
SYGMN

SEQ ID NO: 264 - protein, 17 aa, synthetic construct
  source 1..17: note = F07[PSMA] vhCDR2
VTSYDGSNKYYADSVKG

SEQ ID NO: 265 - protein, 13 aa, synthetic construct
  source 1..13: note = F07[PSMA] vhCDR3
DPYSSSWNGAFDI

SEQ ID NO: 266 - protein, 108 aa, synthetic construct
  source 1..108: note = F07[PSMA] Variable Light (vl) Domain
SYELTQPPSVSVAPGQTARITCGGNNIGSKSVHWYQQKPGQAPVLVVYDDSDRPSGIPERFSGSNSGNTATLTISRVEAGDEADYYCQVWDSSTDHVVFGGGTKLTVL

SEQ ID NO: 267 - protein, 11 aa, synthetic construct
  source 1..11: note = F07[PSMA] vlCDR1
GGNNIGSKSVH

SEQ ID NO: 268 - protein, 7 aa, synthetic construct
  source 1..7: note = F07[PSMA] vlCDR2
DDSDRPS

SEQ ID NO: 269 - protein, 11 aa, synthetic construct
  source 1..11: note = F07[PSMA] vlCDR3
QVWDSSTDHVV

SEQ ID NO: 270 - protein, 122 aa, synthetic construct
  source 1..122: note = F07v2[PSMA] Variable Heavy (vh) Domain
EVQLVESGGGVVQPGRSLRLSCAASGFTFSSYGMNWVRQAPGKGLEWVAVTSYDESNKYYADSVKGRFTISRDISKNTLYLQMSSLRAEDTAVYYCARDPYSSSWNGAFDIWGPGTMVTVSS

SEQ ID NO: 271 - protein, 5 aa, synthetic construct
  source 1..5: note = F07v2[PSMA] vhCDR1
SYGMN

SEQ ID NO: 272 - protein, 17 aa, synthetic construct
  source 1..17: note = F07v2[PSMA] vhCDR2
VTSYDESNKYYADSVKG

SEQ ID NO: 273 - protein, 13 aa, synthetic construct
  source 1..13: note = F07v2[PSMA] vhCDR3
DPYSSSWNGAFDI

SEQ ID NO: 274 - protein, 108 aa, synthetic construct; identical to SEQ ID NO: 266
  source 1..108: note = F07v2[PSMA] Variable Light (vl) Domain

SEQ ID NO: 275 - protein, 11 aa, synthetic construct
  source 1..11: note = F07v2[PSMA] vlCDR1
GGNNIGSKSVH

SEQ ID NO: 276 - protein, 7 aa, synthetic construct
  source 1..7: note = F07v2[PSMA] vlCDR2
DDSDRPS

SEQ ID NO: 277 - protein, 11 aa, synthetic construct
  source 1..11: note = F07v2[PSMA] vlCDR3
QVWDSSTDHVV

SEQ ID NO: 278 - protein, 124 aa, synthetic construct
  source 1..124: note = G02[PSMA] Variable Heavy (vh) Domain
EVQLVESGGGVVQPGRSLRLSCAASGFSFSGYGMHWVRQAPGKGLEWVAVISYDGSNKYYADSVKGRFTISRDNSKNTLYLQMNSLRVEDTAVYYCARDRIWGSRGYYYGMDVWGQGTTVTVSS

SEQ ID NO: 279 - protein, 5 aa, synthetic construct
  source 1..5: note = G02[PSMA] vhCDR1
GYGMH

SEQ ID NO: 280 - protein, 17 aa, synthetic construct
  source 1..17: note = G02[PSMA] vhCDR2
VISYDGSNKYYADSVKG

SEQ ID NO: 281 - protein, 15 aa, synthetic construct
  source 1..15: note = G02[PSMA] vhCDR3
DRIWGSRGYYYGMDV

SEQ ID NO: 282 - protein, 110 aa, synthetic construct
  source 1..110: note = G02[PSMA] Variable Light (vl) Domain
QSALTQPASVSGSPGQSITISCTGASSDVGGYNYVSWYQQHPGKAPKLMIYEVSNRPSGVSNRFSGSKSGNTASLTISGLQAEDEADYYCSSYTITSTLVFGGGTKLTVL

SEQ ID NO: 283 - protein, 14 aa, synthetic construct
  source 1..14: note = G02[PSMA] vlCDR1
TGASSDVGGYNYVS

SEQ ID NO: 284 - protein, 7 aa, synthetic construct
  source 1..7: note = G02[PSMA] vlCDR2
EVSNRPS

SEQ ID NO: 285 - protein, 10 aa, synthetic construct
  source 1..10: note = G02[PSMA] vlCDR3
SSYTITSTLV

SEQ ID NO: 286 - protein, 124 aa, synthetic construct
  source 1..124: note = G02v2[PSMA] Variable Heavy (vh) Domain
EVQLVESGGGVVQPGRSLRLSCAASGFSFSGYGMHWVRQAPGKGLEWVAVISYDESNKYYADSVKGRFTISRDNSKNTLYLQMNSLRVEDTAVYYCARDRIWGSRGYYYGMDVWGQGTTVTVSS

SEQ ID NO: 287 - protein, 5 aa, synthetic construct
  source 1..5: note = G02v2[PSMA] vhCDR1
GYGMH

SEQ ID NO: 288 - protein, 17 aa, synthetic construct
  source 1..17: note = G02v2[PSMA] vhCDR2
VISYDESNKYYADSVKG

SEQ ID NO: 289 - protein, 15 aa, synthetic construct
  source 1..15: note = G02v2[PSMA] vhCDR3
DRIWGSRGYYYGMDV

SEQ ID NO: 290 - protein, 110 aa, synthetic construct; identical to SEQ ID NO: 282
  source 1..110: note = G02v2[PSMA] Variable Light (vl) Domain

SEQ ID NO: 291 - protein, 14 aa, synthetic construct
  source 1..14: note = G02v2[PSMA] vlCDR1
TGASSDVGGYNYVS

SEQ ID NO: 292 - protein, 7 aa, synthetic construct
  source 1..7: note = G02v2[PSMA] vlCDR2
EVSNRPS

SEQ ID NO: 293 - protein, 10 aa, synthetic construct
  source 1..10: note = G02v2[PSMA] vlCDR3
SSYTITSTLV

SEQ ID NO: 294 - protein, 122 aa, synthetic construct
  source 1..122: note = F01[PSMA] Variable Heavy (vh) Domain
QVQLQESGGGVVQPGRSLRLSCAASGFTFSTYGMHWVRQAPGKGLEWVAFISYDGSNKYYADSVKGRFTISRDNSKHTLYLQMNSLRAEDTAVYYCAGRDNLRFLEWFMDVWGQGTTVTVSS

SEQ ID NO: 295 - protein, 5 aa, synthetic construct
  source 1..5: note = F01[PSMA] vhCDR1
TYGMH

SEQ ID NO: 296 - protein, 17 aa, synthetic construct
  source 1..17: note = F01[PSMA] vhCDR2
FISYDGSNKYYADSVKG

SEQ ID NO: 297 - protein, 13 aa, synthetic construct
  source 1..13: note = F01[PSMA] vhCDR3
RDNLRFLEWFMDV

SEQ ID NO: 298 - protein, 108 aa, synthetic construct
  source 1..108: note = F01[PSMA] Variable Light (vl) Domain
EIVLTQSPGTLSVSPGERATLSCRASQSVRSNLAWYQQKPGQAPRLLIYGASTRATGIPARFSGSGSGTEFTLTISSLQSEDFAVYYCHQYNDWPPYTFGQGTKLEIK

SEQ ID NO: 299 - protein, 11 aa, synthetic construct
  source 1..11: note = F01[PSMA] vlCDR1
RASQSVRSNLA

SEQ ID NO: 300 - protein, 7 aa, synthetic construct
  source 1..7: note = F01[PSMA] vlCDR2
GASTRAT

SEQ ID NO: 301 - protein, 10 aa, synthetic construct
  source 1..10: note = F01[PSMA] vlCDR3
HQYNDWPPYT

SEQ ID NO: 302 - protein, 122 aa, synthetic construct
  source 1..122: note = F01v2[PSMA] Variable Heavy (vh) Domain
QVQLQESGGGVVQPGRSLRLSCAASGFTFSTYGMHWVRQAPGKGLEWVAFISYDESNKYYADSVKGRFTISRDNSKHTLYLQMNSLRAEDTAVYYCAGRDNLRFLEWFMDVWGQGTTVTVSS

SEQ ID NO: 303 - protein, 5 aa, synthetic construct
  source 1..5: note = F01v2[PSMA] vhCDR1
TYGMH

SEQ ID NO: 304 - protein, 17 aa, synthetic construct
  source 1..17: note = F01v2[PSMA] vhCDR2
FISYDESNKYYADSVKG

SEQ ID NO: 305 - protein, 13 aa, synthetic construct
  source 1..13: note = F01v2[PSMA] vhCDR3
RDNLRFLEWFMDV

SEQ ID NO: 306 - protein, 108 aa, synthetic construct; identical to SEQ ID NO: 298
  source 1..108: note = F01v2[PSMA] Variable Light (vl) Domain

SEQ ID NO: 307 - protein, 11 aa, synthetic construct
  source 1..11: note = F01v2[PSMA] vlCDR1
RASQSVRSNLA

SEQ ID NO: 308 - protein, 7 aa, synthetic construct
  source 1..7: note = F01v2[PSMA] vlCDR2
GASTRAT

SEQ ID NO: 309 - protein, 10 aa, synthetic construct
  source 1..10: note = F01v2[PSMA] vlCDR3
HQYNDWPPYT

SEQ ID NO: 310 - protein, 120 aa, synthetic construct
  source 1..120: note = 011A11[PSMA] Variable Heavy (vh) Domain
EVQLVESGGGLVKPGGSLRLSCVASGFTFSFYSMNWVRQAPGKGLDWVSSISSSGNYIYYADSVKGRFTISRDNAKNSLHLHMNSLKAEDTAMYFCARSYSGSYDAFDFWGQGTMVTVSS

SEQ ID NO: 311 - protein, 5 aa, synthetic construct
  source 1..5: note = 011A11[PSMA] vhCDR1
FYSMN

SEQ ID NO: 312 - protein, 17 aa, synthetic construct
  source 1..17: note = 011A11[PSMA] vhCDR2
SISSSGNYIYYADSVKG

SEQ ID NO: 313 - protein, 11 aa, synthetic construct
  source 1..11: note = 011A11[PSMA] vhCDR3
SYSGSYDAFDF

SEQ ID NO: 314 - protein, 108 aa, synthetic construct
  source 1..108: note = 011A11[PSMA] Variable Light (vl) Domain
EIVMTQSPGTLSLSPGERATLSCRASQSVSSSFLAWYQQKPGQAPRLLISGASSRATGIPDRFSVSGSGTDFTLTISRLEPEDFAVYYCQQYGVSPWTFGQGTKVEIK

SEQ ID NO: 315 - protein, 12 aa, synthetic construct
  source 1..12: note = 011A11[PSMA] vlCDR1
RASQSVSSSFLA

SEQ ID NO: 316 - protein, 7 aa, synthetic construct
  source 1..7: note = 011A11[PSMA] vlCDR2
GASSRAT

SEQ ID NO: 317 - protein, 9 aa, synthetic construct
  source 1..9: note = 011A11[PSMA] vlCDR3
QQYGVSPWT

SEQ ID NO: 318 - protein, 121 aa, synthetic construct
  source 1..121: note = PSMB896[PSMA] Variable Heavy (vh) Domain
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSS

SEQ ID NO: 319 - protein, 5 aa, synthetic construct
  source 1..5: note = PSMB896[PSMA] vhCDR1
SYAMS

SEQ ID NO: 320 - protein, 17 aa, synthetic construct
  source 1..17: note = PSMB896[PSMA] vhCDR2
AISGGIGSTYYADSVKG

SEQ ID NO: 321 - protein, 12 aa, synthetic construct
  source 1..12: note = PSMB896[PSMA] vhCDR3
DAVGATPYYFDY

SEQ ID NO: 322 - protein, 110 aa, synthetic construct
  source 1..110: note = PSMB896[PSMA] Variable Light (vl) Domain
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVL

SEQ ID NO: 323 - protein, 13 aa, synthetic construct
  source 1..13: note = PSMB896[PSMA] vlCDR1
SGSSSNIGINYVS

SEQ ID NO: 324 - protein, 7 aa, synthetic construct
  source 1..7: note = PSMB896[PSMA] vlCDR2
DNNKRPS

SEQ ID NO: 325 - protein, 11 aa, synthetic construct
  source 1..11: note = PSMB896[PSMA] vlCDR3
GTWDSSLSAVV

SEQ ID NO: 326 - protein, 115 aa, synthetic construct
  source 1..115: note = PSMA-H[PSMA] Variable Heavy (vh) Domain
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSS

SEQ ID NO: 327 - protein, 5 aa, synthetic construct
  source 1..5: note = PSMA-H[PSMA] vhCDR1
EYTIH

SEQ ID NO: 328 - protein, 17 aa, synthetic construct
  source 1..17: note = PSMA-H[PSMA] vhCDR2
NINPNNGGTTYNQKFQG

SEQ ID NO: 329 - protein, 6 aa, synthetic construct
  source 1..6: note = PSMA-H[PSMA] vhCDR3
GWNFDY

SEQ ID NO: 330 - protein, 107 aa, synthetic construct
  source 1..107: note = PSMA-H[PSMA] Variable Light (vl) Domain
DIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIK

SEQ ID NO: 331 - protein, 11 aa, synthetic construct
  source 1..11: note = PSMA-H[PSMA] vlCDR1
RASQDVGTAVD

SEQ ID NO: 332 - protein, 7 aa, synthetic construct
  source 1..7: note = PSMA-H[PSMA] vlCDR2
WASTRHT

SEQ ID NO: 333 - protein, 9 aa, synthetic construct
  source 1..9: note = PSMA-H[PSMA] vlCDR3
QQYNSYPLT

SEQ ID NO: 334 - protein, 120 aa, synthetic construct
  source 1..120: note = D7[PSMA] Variable Heavy (vh) Domain
QVQLQQSGAELVEPGASVKLSCKASGYTFTYFDINWLRQRPEQGLEWIGGISPGDGNTNYNENFKGKATLTIDKSSTTAYIQLSRLTSEDSAVYFCARDGNFPYYAMDSWGQGTSVTVSS

SEQ ID NO: 335 - protein, 5 aa, synthetic construct
  source 1..5: note = D7[PSMA] vhCDR1
YFDIN

SEQ ID NO: 336 - protein, 17 aa, synthetic construct
  source 1..17: note = D7[PSMA] vhCDR2
GISPGDGNTNYNENFKG

SEQ ID NO: 337 - protein, 11 aa, synthetic construct
  source 1..11: note = D7[PSMA] vhCDR3
DGNFPYYAMDS

SEQ ID NO: 338 - protein, 111 aa, synthetic construct
  source 1..111: note = D7[PSMA] Variable Light (vl) Domain
DIELTQSPLSLPVILGDQASISCRSSQSLVHSNGNTYLHWFLQKPGQSPKLLIYTVSNRFSGVPDRFSGSGSGTDFTLKISRVEAEDLGVYFCSQSTHVPTFGGGTKLEIK

SEQ ID NO: 339 - protein, 16 aa, synthetic construct
  source 1..16: note = D7[PSMA] vlCDR1
RSSQSLVHSNGNTYLH

SEQ ID NO: 340 - protein, 7 aa, synthetic construct
  source 1..7: note = D7[PSMA] vlCDR2
TVSNRFS

SEQ ID NO: 341 - protein, 8 aa, synthetic construct
  source 1..8: note = D7[PSMA] vlCDR3
SQSTHVPT

SEQ ID NO: 342 - protein, 478 aa, synthetic construct
  source 1..478: note = C28PB397 Chain 1 - 1A7[CD28]_H1_L1.71 spFv
EVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 450 aa, synthetic construct
  source 1..450: note = C28PB397 Chain 2 - HC1 (hole3 RF): PSMA_P72_A10V2
QVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 344 - protein, 213 aa, synthetic construct
  source 1..213: note = C28PB397 Chain 3 - LC: PSMA_P72_A10V2
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 345 - protein, 477 aa, synthetic construct
  source 1..477: note = C28PB397[(K447del)] Chain 1 - 1A7[CD28]_H1_L1.71 spFv (K447del)
EVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 346 - protein, 449 aa, synthetic construct
  source 1..449: note = C28PB397[(K447del)] Chain 2 - HC1 (hole3 RF): PSMA_P72_A10V2 (K447del)
QVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 347 - protein, 213 aa, synthetic construct; identical to SEQ ID NO: 344
  source 1..213: note = C28PB397[(K447del)] Chain 3 - LC: PSMA_P72_A10V2

SEQ ID NO: 348 - protein, 476 aa, synthetic construct
  source 1..476: note = C28PB397[(G446del/K447del)] Chain 1 - 1A7[CD28]_H1_L1.71 spFv (G446del/K447del)
EVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 349 - protein, 448 aa, synthetic construct
  source 1..448: note = C28PB397[(G446del/K447del)] Chain 2 - HC1 (hole3 RF): PSMA_P72_A10V2 (G446del/K447del)
QVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 350 - protein, 213 aa, synthetic construct; identical to SEQ ID NO: 344
  source 1..213: note = C28PB397[(G446del/K447del)] Chain 3 - LC: PSMA_P72_A10V2

SEQ ID NO: 351 - protein, 480 aa, synthetic construct
  source 1..480: note = C28PB330 Chain 1 - 1A7[CD28]_H1_L1.71-HL-scFv
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 450 aa, synthetic construct; identical to SEQ ID NO: 343
  source 1..450: note = C28PB330 Chain 2 - HC1 (knob): PSMA_P72_A10V2

SEQ ID NO: 353 - protein, 213 aa, synthetic construct; identical to SEQ ID NO: 344
  source 1..213: note = C28PB330 Chain 3 - LC: PSMA_P72_A10V2

SEQ ID NO: 354 - protein, 479 aa, synthetic construct
  source 1..479: note = C28PB330[(K447del)] Chain 1 - 1A7[CD28]_H1_L1.71-HL-scFv (K447del)
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 355 - protein, 449 aa, synthetic construct; identical to SEQ ID NO: 346
  source 1..449: note = C28PB330[(K447del)] Chain 2 - HC1 (knob): PSMA_P72_A10V2 (K447del)

SEQ ID NO: 356 - protein, 213 aa, synthetic construct; identical to SEQ ID NO: 344
  source 1..213: note = C28PB330[(K447del)] Chain 3 - LC: PSMA_P72_A10V2

SEQ ID NO: 357 - protein, 478 aa, synthetic construct
  source 1..478: note = C28PB330[(G446del/K447del)] Chain 1 - 1A7[CD28]_H1_L1.71-HL-scFv (G446del/K447del)
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 358 - protein, 448 aa, synthetic construct; identical to SEQ ID NO: 349
  source 1..448: note = C28PB330[(G446del/K447del)] Chain 2 - HC1 (knob): PSMA_P72_A10V2 (G446del/K447del)

SEQ ID NO: 359 - protein, 213 aa, synthetic construct; identical to SEQ ID NO: 344
  source 1..213: note = C28PB330[(G446del/K447del)] Chain 3 - LC: PSMA_P72_A10V2

SEQ ID NO: 360 - protein, 121 aa, synthetic construct
  source 1..121: note = 1A7_H1sp Variable heavy (vh) domain
EVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSS

SEQ ID NO: 361 - protein, 5 aa, synthetic construct
  source 1..5: note = 1A7_H1sp vhCDR1
SYAMS

SEQ ID NO: 362 - protein, 17 aa, synthetic construct
  source 1..17: note = 1A7_H1sp vhCDR2
TISGSGDSTYYADSVKG

SEQ ID NO: 363 - protein, 12 aa, synthetic construct
  source 1..12: note = 1A7_H1sp vhCDR3
SGPGLRQVGFDY

SEQ ID NO: 364 - protein, 121 aa, synthetic construct
  source 1..121: note = 1A7_H1.1sp Variable heavy (vh) domain
EVQLLESGGGLVQPGGSLRLSCAASGFTFSSYYMSWVRQAPGCGLEWVSTISGSGDSTYYADSVKGRFTISRDNSKNTLYLQMNSLRAEDTAVYYCAKSGPGLRQVGFDYWGQGTLVTVSS

SEQ ID NO: 365 - protein, 5 aa, synthetic construct
  source 1..5: note = 1A7_H1.1sp vhCDR1
SYYMS

SEQ ID NO: 366 - protein, 17 aa, synthetic construct
  source 1..17: note = 1A7_H1.1sp vhCDR2
TISGSGDSTYYADSVKG

SEQ ID NO: 367 - protein, 12 aa, synthetic construct
  source 1..12: note = 1A7_H1.1sp vhCDR3
SGPGLRQVGFDY

SEQ ID NO: 368 - protein, 121 aa, synthetic construct
  source 1..121: note = 1A7_H1.14sp Variable heavy (vh) domain
EVQLLESGGGLVQPGGSLRLSCAASGFTFSSYYMSWVRQAPGCGLEWVSTISESGDSTYYADSVKGRFTISRDNSKNTLYLQMNSLRAEDTAVYYCAKSGPGLRQVGFDYWGQGTLVTVSS

SEQ ID NO: 369 - protein, 5 aa, synthetic construct
  source 1..5: note = 1A7_H1.14sp vhCDR1
SYYMS

SEQ ID NO: 370 - protein, 17 aa, synthetic construct
  source 1..17: note = 1A7_H1.14sp vhCDR2
TISESGDSTYYADSVKG

SEQ ID NO: 371 - protein, 12 aa, synthetic construct
  source 1..12: note = 1A7_H1.14sp vhCDR3
SGPGLRQVGFDY

SEQ ID NO: 372 - protein, 107 aa, synthetic construct
  source 1..107: note = 1A7_L1sp Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGCGTKLEIK

SEQ ID NO: 373 - protein, 11 aa, synthetic construct
  source 1..11: note = 1A7_L1sp vlCDR1
RASQSISSYLN

SEQ ID NO: 374 - protein, 7 aa, synthetic construct
  source 1..7: note = 1A7_L1sp vlCDR2
AASSLQS

SEQ ID NO: 375 - protein, 9 aa, synthetic construct
  source 1..9: note = 1A7_L1sp vlCDR3
QQSYSTPFT

SEQ ID NO: 376 - protein, 107 aa, synthetic construct
  source 1..107: note = 1A7_L1.71sp Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGCGTKLEIK

SEQ ID NO: 377 - protein, 11 aa, synthetic construct
  source 1..11: note = 1A7_L1.71sp vlCDR1
RASQSISSYLN

SEQ ID NO: 378 - protein, 7 aa, synthetic construct
  source 1..7: note = 1A7_L1.71sp vlCDR2
AASSLQS

SEQ ID NO: 379 - protein, 9 aa, synthetic construct
  source 1..9: note = 1A7_L1.71sp vlCDR3
QQVYSTPFT

SEQ ID NO: 380 - protein, 121 aa, synthetic construct; identical to SEQ ID NO: 360
  source 1..121: note = 1A7[CD28]_H1sp_L1sp Variable Heavy (vh) Domain

SEQ ID NO: 381 - protein, 5 aa, synthetic construct
  source 1..5: note = 1A7[CD28]_H1sp_L1sp vhCDR1
SYAMS

SEQ ID NO: 382 - protein, 17 aa, synthetic construct
  source 1..17: note = 1A7[CD28]_H1sp_L1sp vhCDR2
TISGSGDSTYYADSVKG

SEQ ID NO: 383 - protein, 12 aa, synthetic construct
  source 1..12: note = 1A7[CD28]_H1sp_L1sp vhCDR3
SGPGLRQVGFDY

SEQ ID NO: 384 - protein, 107 aa, synthetic construct; identical to SEQ ID NO: 372
  source 1..107: note = 1A7[CD28]_H1sp_L1sp Variable Light (vl) Domain

SEQ ID NO: 385 - protein, 11 aa, synthetic construct
  source 1..11: note = 1A7[CD28]_H1sp_L1sp vlCDR1
RASQSISSYLN

SEQ ID NO: 386 - protein, 7 aa, synthetic construct
  source 1..7: note = 1A7[CD28]_H1sp_L1sp vlCDR2
AASSLQS

SEQ ID NO: 387 - protein, 9 aa, synthetic construct
  source 1..9: note = 1A7[CD28]_H1sp_L1sp vlCDR3
QQSYSTPFT

SEQ ID NO: 388 - protein, 121 aa, synthetic construct; identical to SEQ ID NO: 364
  source 1..121: note = 1A7[CD28]_H1.1sp_L1sp Variable Heavy (vh) Domain

SEQ ID NO: 389 - protein, 5 aa, synthetic construct
  source 1..5: note = 1A7[CD28]_H1.1sp_L1sp vhCDR1
SYYMS

SEQ ID NO: 390 - protein, 17 aa, synthetic construct
  source 1..17: note = 1A7[CD28]_H1.1sp_L1sp vhCDR2
TISGSGDSTYYADSVKG

SEQ ID NO: 391 - protein, 12 aa, synthetic construct
  source 1..12: note = 1A7[CD28]_H1.1sp_L1sp vhCDR3
SGPGLRQVGFDY

SEQ ID NO: 392 - protein, 107 aa, synthetic construct; identical to SEQ ID NO: 372
  source 1..107: note = 1A7[CD28]_H1.1sp_L1sp Variable Light (vl) Domain

SEQ ID NO: 393 - protein, 11 aa, synthetic construct
  source 1..11: note = 1A7[CD28]_H1.1sp_L1sp vlCDR1
RASQSISSYLN

SEQ ID NO: 394 - protein, 7 aa, synthetic construct
  source 1..7: note = 1A7[CD28]_H1.1sp_L1sp vlCDR2
AASSLQS

SEQ ID NO: 395 - protein, 9 aa, synthetic construct
  source 1..9: note = 1A7[CD28]_H1.1sp_L1sp vlCDR3
QQSYSTPFT

SEQ ID NO: 396 - protein, 121 aa, synthetic construct; identical to SEQ ID NO: 360
  source 1..121: note = 1A7[CD28]_H1sp_L1.71sp Variable Heavy (vh) Domain

SEQ ID NO: 397 - protein, 5 aa, synthetic construct
  source 1..5: note = 1A7[CD28]_H1sp_L1.71sp vhCDR1
SYAMS

SEQ ID NO: 398 - protein, 17 aa, synthetic construct
  source 1..17: note = 1A7[CD28]_H1sp_L1.71sp vhCDR2
TISGSGDSTYYADSVKG

SEQ ID NO: 399 - protein, 12 aa, synthetic construct
  source 1..12: note = 1A7[CD28]_H1sp_L1.71sp vhCDR3
SGPGLRQVGFDY

SEQ ID NO: 400 - protein, 107 aa, synthetic construct; identical to SEQ ID NO: 376
  source 1..107: note = 1A7[CD28]_H1sp_L1.71sp Variable Light (vl) Domain

SEQ ID NO: 401 - protein, 11 aa, synthetic construct
  source 1..11: note = 1A7[CD28]_H1sp_L1.71sp vlCDR1
RASQSISSYLN

SEQ ID NO: 402 - protein, 7 aa, synthetic construct
  source 1..7: note = 1A7[CD28]_H1sp_L1.71sp vlCDR2
AASSLQS

SEQ ID NO: 403 - protein, 9 aa, synthetic construct
  source 1..9: note = 1A7[CD28]_H1sp_L1.71sp vlCDR3
QQVYSTPFT

SEQ ID NO: 404 - protein, 121 aa, synthetic construct; identical to SEQ ID NO: 364
  source 1..121: note = 1A7[CD28]_H1.1sp_L1.71sp Variable Heavy (vh) Domain

SEQ ID NO: 405 - protein, 5 aa, synthetic construct
  source 1..5: note = 1A7[CD28]_H1.1sp_L1.71sp vhCDR1
SYYMS

SEQ ID NO: 406 - protein, 17 aa, synthetic construct
  source 1..17: note = 1A7[CD28]_H1.1sp_L1.71sp vhCDR2
TISGSGDSTYYADSVKG

SEQ ID NO: 407 - protein, 12 aa, synthetic construct
  source 1..12: note = 1A7[CD28]_H1.1sp_L1.71sp vhCDR3
SGPGLRQVGFDY

SEQ ID NO: 408 - protein, 107 aa, synthetic construct; identical to SEQ ID NO: 376
  source 1..107: note = 1A7[CD28]_H1.1sp_L1.71sp Variable Light (vl) Domain

SEQ ID NO: 409 - protein, 11 aa, synthetic construct
  source 1..11: note = 1A7[CD28]_H1.1sp_L1.71sp vlCDR1
RASQSISSYLN

SEQ ID NO: 410 - protein, 7 aa, synthetic construct
  source 1..7: note = 1A7[CD28]_H1.1sp_L1.71sp vlCDR2
AASSLQS

SEQ ID NO: 411 - protein, 9 aa, synthetic construct
  source 1..9: note = 1A7[CD28]_H1.1sp_L1.71sp vlCDR3
QQVYSTPFT

SEQ ID NO: 412 - protein, 121 aa, synthetic construct; identical to SEQ ID NO: 368
  source 1..121: note = 1A7[CD28]_H1.14sp_L1sp Variable Heavy (vh) Domain

SEQ ID NO: 413 - protein, 5 aa, synthetic construct
  source 1..5: note = 1A7[CD28]_H1.14sp_L1sp vhCDR1
SYYMS

SEQ ID NO: 414 - protein, 17 aa, synthetic construct
  source 1..17: note = 1A7[CD28]_H1.14sp_L1sp vhCDR2
TISESGDSTYYADSVKG

SEQ ID NO: 415 - protein, 12 aa, synthetic construct
  source 1..12: note = 1A7[CD28]_H1.14sp_L1sp vhCDR3
SGPGLRQVGFDY

SEQ ID NO: 416 - protein, 107 aa, synthetic construct; identical to SEQ ID NO: 372
  source 1..107: note = 1A7[CD28]_H1.14sp_L1sp Variable Light (vl) Domain

SEQ ID NO: 417 - protein, 11 aa, synthetic construct
  source 1..11: note = 1A7[CD28]_H1.14sp_L1sp vlCDR1
RASQSISSYLN

SEQ ID NO: 418 - protein, 7 aa, synthetic construct
  source 1..7: note = 1A7[CD28]_H1.14sp_L1sp vlCDR2
AASSLQS

SEQ ID NO: 419 - protein, 9 aa, synthetic construct
  source 1..9: note = 1A7[CD28]_H1.14sp_L1sp vlCDR3
QQSYSTPFT

SEQ ID NO: 420 - protein, 121 aa, synthetic construct; identical to SEQ ID NO: 368
  source 1..121: note = 1A7[CD28]_H1.14sp_L1.71sp Variable Heavy (vh) Domain

SEQ ID NO: 421 - protein, 5 aa, synthetic construct
  source 1..5: note = 1A7[CD28]_H1.14sp_L1.71sp vhCDR1
SYYMS

SEQ ID NO: 422 - protein, 17 aa, synthetic construct
  source 1..17: note = 1A7[CD28]_H1.14sp_L1.71sp vhCDR2
TISESGDSTYYADSVKG

SEQ ID NO: 423 - protein, 12 aa, synthetic construct
  source 1..12: note = 1A7[CD28]_H1.14sp_L1.71sp vhCDR3
SGPGLRQVGFDY

SEQ ID NO: 424 - protein, 107 aa, synthetic construct; identical to SEQ ID NO: 376
  source 1..107: note = 1A7[CD28]_H1.14sp_L1.71sp Variable Light (vl) Domain

SEQ ID NO: 425 - protein, 11 aa, synthetic construct
  source 1..11: note = 1A7[CD28]_H1.14sp_L1.71sp vlCDR1
RASQSISSYLN

SEQ ID NO: 426 - protein, 7 aa, synthetic construct
  source 1..7: note = 1A7[CD28]_H1.14sp_L1.71sp vlCDR2
AASSLQS

SEQ ID NO: 427 - protein, 9 aa, synthetic construct
  source 1..9: note = 1A7[CD28]_H1.14sp_L1.71sp vlCDR3
QQVYSTPFT

SEQ ID NO: 428 - protein, 750 aa, Homo sapiens
  source 1..750: note = sp|Q04609
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 429 - protein, 707 aa, Homo sapiens
  source 1..707: note = sp|Q04609|44-750
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 430 - protein, 752 aa, Mus musculus
  source 1..752: note = sp|O35409
MWNALQDRDSAEVLGHRQRWLRVGTLVLALTGTFLIGFLFGWFIKPSNEATGNVSHSGMKKEFLHELKAENIKKFLYNFTRTPHLAGTQNNFELAKQIHDQWKEFGLDLVELSHYDVLLSYPNKTHPNYISIINEDGNEIFKTSLSEQPPPGYENISDVVPPYSAFSPQGTPEGDLVYVNYARTEDFFKLEREMKISCSGKIVIARYGKVFRGNMVKNAQLAGAKGMILYSDPADYFVPAVKSYPDGWNLPGGGVQRGNVLNLNGAGDPLTPGYPANEHAYRHELTNAVGLPSIPVHPIGYDDAQKLLEHMGGPAPPDSSWKGGLKVPYNVGPGFAGNFSTQKVKMHIHSYTKVTRIYNVIGTLKGALEPDRYVILGGHRDAWVFGGIDPQSGAAVVHEIVRSFGTLKKKGRRPRRTILFASWDAEEFGLLGSTEWAEEHSRLLQERGVAYINADSSIEGNYTLRVDCTPLMYSLVYNLTKELQSPDEGFEGKSLYDSWKEKSPSPEFIGMPRISKLGSGNDFEVFFQRLGIASGRARYTKNWKTNKVSSYPLYHSVYETYELVVKFYDPTFKYHLTVAQVRGAMVFELANSIVLPFDCQSYAVALKKYADTIYNISMKHPQEMKAYMISFDSLFSAVNNFTDVASKFNQRLQELDKSNPILLRIMNDQLMYLERAFIDPLGLPGRPFYRHIIYAPSSHNKYAGESFPGIYDALFDISSKVNASKAWNEVKRQISIATFTVQAAAETLREVA

SEQ ID NO: 431 - protein, 708 aa, Mus musculus
  source 1..708: note = sp|O35409|45-752
KPSNEATGNVSHSGMKKEFLHELKAENIKKFLYNFTRTPHLAGTQNNFELAKQIHDQWKEFGLDLVELSHYDVLLSYPNKTHPNYISIINEDGNEIFKTSLSEQPPPGYENISDVVPPYSAFSPQGTPEGDLVYVNYARTEDFFKLEREMKISCSGKIVIARYGKVFRGNMVKNAQLAGAKGMILYSDPADYFVPAVKSYPDGWNLPGGGVQRGNVLNLNGAGDPLTPGYPANEHAYRHELTNAVGLPSIPVHPIGYDDAQKLLEHMGGPAPPDSSWKGGLKVPYNVGPGFAGNFSTQKVKMHIHSYTKVTRIYNVIGTLKGALEPDRYVILGGHRDAWVFGGIDPQSGAAVVHEIVRSFGTLKKKGRRPRRTILFASWDAEEFGLLGSTEWAEEHSRLLQERGVAYINADSSIEGNYTLRVDCTPLMYSLVYNLTKELQSPDEGFEGKSLYDSWKEKSPSPEFIGMPRISKLGSGNDFEVFFQRLGIASGRARYTKNWKTNKVSSYPLYHSVYETYELVVKFYDPTFKYHLTVAQVRGAMVFELANSIVLPFDCQSYAVALKKYADTIYNISMKHPQEMKAYMISFDSLFSAVNNFTDVASKFNQRLQELDKSNPILLRIMNDQLMYLERAFIDPLGLPGRPFYRHIIYAPSSHNKYAGESFPGIYDALFDISSKVNASKAWNEVKRQISIATFTVQAAAETLREVA

SEQ ID NO: 432 - protein, 750 aa, Macaca fascicularis
  source 1..750: note = tr|G7PNF
MWNLLHETDSAVATARRPRWLCAGALVLAGGFFLLGFLFGWFIKSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPDDYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGMLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 433 - protein, 707 aa, Macaca fascicularis
  source 1..707: note = tr|G7PNF|44-750
KSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPDDYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGMLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 434 - protein, 15 aa, synthetic construct
GGGGSGGGGSGGGGS

SEQ ID NO: 435 - protein, 18 aa, synthetic construct
GSTSGSGKPGSGEGSTKG

SEQ ID NO: 436 - protein, 14 aa, synthetic construct
IRPRAIGGSKPRVA

SEQ ID NO: 437 - protein, 15 aa, synthetic construct
GKGGSGKGGSGKGGS

SEQ ID NO: 438 - protein, 15 aa, synthetic construct
GGKGSGGKGSGGKGS

SEQ ID NO: 439 - protein, 15 aa, synthetic construct
GGGKSGGGKSGGGKS

SEQ ID NO: 440 - protein, 15 aa, synthetic construct
GKGKSGKGKSGKGKS

SEQ ID NO: 441 - protein, 15 aa, synthetic construct
GGGKSGGKGSGKGGS

SEQ ID NO: 442 - protein, 15 aa, synthetic construct
GKPGSGKPGSGKPGS

SEQ ID NO: 443 - protein, 20 aa, synthetic construct
GKPGSGKPGSGKPGSGKPGS

SEQ ID NO: 444 - protein, 20 aa, synthetic construct
GKGKSGKGKSGKGKSGKGKS

SEQ ID NO: 445 - protein, 20 aa, synthetic construct
GGGGSGGGGSGGGGSGGGGS

SEQ ID NO: 446 - protein, 14 aa, synthetic construct
STAGDTHLGGEDFD

SEQ ID NO: 447 - protein, 15 aa, synthetic construct
GEGGSGEGGSGEGGS

SEQ ID NO: 448 - protein, 15 aa, synthetic construct
GGEGSGGEGSGGEGS

SEQ ID NO: 449 - protein, 15 aa, synthetic construct
GGGESGGGESGGGES

SEQ ID NO: 450 - protein, 15 aa, synthetic construct
GEGESGEGESGEGES

SEQ ID NO: 451 - protein, 15 aa, synthetic construct
GGGESGGEGSGEGGS

SEQ ID NO: 452 - protein, 20 aa, synthetic construct
GEGESGEGESGEGESGEGES

SEQ ID NO: 453 - protein, 19 aa, synthetic construct
PRGASKSGSASQTGSAPGS

SEQ ID NO: 454 - protein, 19 aa, synthetic construct
GTAAAGAGAAGGAAAGAAG

SEQ ID NO: 455 - protein, 19 aa, synthetic construct
GTSGSSGSGSGGSGSGGGG

SEQ ID NO: 456 - protein, 20 aa, synthetic construct
GGSEGKSSGSGSESKSTGGS

SEQ ID NO: 457 - protein, 18 aa, synthetic construct
GGGSGGSGGCPPCGGSGG

SEQ ID NO: 458 - protein, 5 aa, synthetic construct
GGGGS

SEQ ID NO: 459 - protein, 10 aa, synthetic construct
GGGGSGGGGS

SEQ ID NO: 460 - protein, 25 aa, synthetic construct
GGGGSGGGGSGGGGSGGGGSGGGGS

SEQ ID NO: 461 - protein, 30 aa, synthetic construct
GGGGSGGGGSGGGGSGGGGSGGGGSGGGGS

SEQ ID NO: 462 - protein, 35 aa, synthetic construct
GGGGSGGGGSGGGGSGGGGSGGGGSGGGGSGGGGS

SEQ ID NO: 463 - protein, 5 aa, synthetic construct
GGGGA

SEQ ID NO: 464 - protein, 10 aa, synthetic construct
GGGGAGGGGA

SEQ ID NO: 465 - protein, 15 aa, synthetic construct
GGGGAGGGGAGGGGA

SEQ ID NO: 466 - protein, 20 aa, synthetic construct
GGGGAGGGGAGGGGAGGGGA

SEQ ID NO: 467 - protein, 25 aa, synthetic construct
GGGGAGGGGAGGGGAGGGGAGGGGA

SEQ ID NO: 468 - protein, 30 aa, synthetic construct
GGGGAGGGGAGGGGAGGGGAGGGGAGGGGA

SEQ ID NO: 469 - protein, 35 aa, synthetic construct
GGGGAGGGGAGGGGAGGGGAGGGGAGGGGAGGGGA

SEQ ID NO: 470 - protein, 30 aa, synthetic construct
DPALVHQRPAPPGGGGSGGGGSGGGGSGGG

SEQ ID NO: 471 - protein, 5 aa, synthetic construct
GKPGS

SEQ ID NO: 472 - protein, 25 aa, synthetic construct
GKPGSGKPGSGKPGSGKPGSGKPGS

SEQ ID NO: 473 - protein, 30 aa, synthetic construct
GKPGSGKPGSGKPGSGKPGSGKPGSGKPGS

SEQ ID NO: 474 - protein, 5 aa, synthetic construct
GGGES

SEQ ID NO: 475 - protein, 15 aa, synthetic construct
EPKSCDKTHTCPPCP

SEQ ID NO: 476 - protein, 9 aa, synthetic construct
KTHTCPPCP

SEQ ID NO: 477 - protein, 15 aa, synthetic construct
EPKSSDKTHTCPPCP

SEQ ID NO: 478 - protein, 19 aa, synthetic construct
GGGGSGGGGSKTHTCPPCP

SEQ ID NO: 479 - protein, 19 aa, synthetic construct
GKPGSGKPGSKTHTCPPCP

SEQ ID NO: 480 - protein, 14 aa, synthetic construct
GKPGSKTHTCPPCP

SEQ ID NO: 481 - protein, 5 aa, synthetic construct
EPKSC

SEQ ID NO: 482 - protein, 15 aa, synthetic construct
EPKSCGGGGSGGGGS

SEQ ID NO: 483 - protein, 15 aa, synthetic construct
EPKSCGKPGSGKPGS

SEQ ID NO: 484 - protein, 10 aa, synthetic construct
EPKSCGKPGS

SEQ ID NO: 485 - protein, 216 aa, synthetic construct
  source 1..216: note = Heterodimeric Fc Backbone 1 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 486 - protein, 216 aa, synthetic construct
  source 1..216: note = Heterodimeric Fc Backbone 1 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 216 aa, synthetic construct; identical to SEQ ID NO: 485
  source 1..216: note = Heterodimeric Fc Backbone 2 monomer 1 (-)

SEQ ID NO: 488 - protein, 216 aa, synthetic construct
  source 1..216: note = Heterodimeric Fc Backbone monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 489 - protein, 216 aa, synthetic construct
  source 1..216: note = Heterodimeric Fc Backbone 3 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 490 - protein, 216 aa, synthetic construct; identical to SEQ ID NO: 488
  source 1..216: note = Heterodimeric Fc Backbone 3 monomer 2 (+)

SEQ ID NO: 491 - protein, 216 aa, synthetic construct
  source 1..216: note = Heterodimeric Fc Backbone 4 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 492 - protein, 216 aa, synthetic construct
  source 1..216: note = Heterodimeric Fc Backbone 4 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 493 - protein, 216 aa, synthetic construct
  source 1..216: note = Heterodimeric Fc Backbone 5 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 494 - protein, 216 aa, synthetic construct
  source 1..216: note = Heterodimeric Fc Backbone 5 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 495 - protein, 216 aa, synthetic construct
  source 1..216: note = Heterodimeric Fc Backbone 6 monomer 1 (-)
APPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 496 - protein, 216 aa, synthetic construct
  source 1..216: note = Heterodimeric Fc Backbone 6 monomer 2 (+)
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 497 - protein, 216 aa, synthetic construct
  source 1..216: note = Heterodimeric Fc Backbone 7 monomer 1 (-)
APPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 498 - protein, 216 aa, synthetic construct
  source 1..216: note = Heterodimeric Fc Backbone 7 monomer 2 (+)
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 499 - protein, 217 aa, synthetic construct
  source 1..217: note = Heterodimeric Fc Backbone 8 monomer 1 (-)
APEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 500 - protein, 217 aa, synthetic construct
  source 1..217: note = Heterodimeric Fc Backbone 8 monomer 2 (+)
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 501 - protein, 216 aa, synthetic construct
  source 1..216: note = Heterodimeric Fc Backbone 9 monomer 1 (-)
APPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 502 - protein, 216 aa, synthetic construct
  source 1..216: note = Heterodimeric Fc Backbone 9 monomer 2 (+)
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 503 - protein, 216 aa, synthetic construct
  source 1..216: note = Heterodimeric Fc Backbone 10 monomer 1 (-)
APPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 504 - protein, 216 aa, synthetic construct
  source 1..216: note = Heterodimeric Fc Backbone 10 monomer 2 (+)
APPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 505 - protein, 216 aa, synthetic construct
  source 1..216: note = Heterodimeric Fc Backbone 11 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 506 - protein, 216 aa, synthetic construct
  source 1..216: note = Heterodimeric Fc Backbone 11 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 507 - protein, 216 aa, synthetic construct
  source 1..216: note = Heterodimeric Fc Backbone 12 monomer 1 (-)
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 508 - protein, 216 aa, synthetic construct; identical to SEQ ID NO: 485
  source 1..216: note = Heterodimeric Fc Backbone 12 monomer 2 (+)

SEQ ID NO: 509 - protein, 217 aa, synthetic construct
  source 1..217: note = Heterodimeric Fc Backbone 13 monomer 1
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 510 - protein, 217 aa, synthetic construct
  source 1..217: note = Heterodimeric Fc Backbone 13 monomer 2
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 511 - protein, 217 aa, synthetic construct
  source 1..217: note = Heterodimeric Fc Backbone 14 monomer 1
APEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 512 - protein, 217 aa, synthetic construct
  source 1..217: note = Heterodimeric Fc Backbone 14 monomer 2
APEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 513 - protein, 216 aa, synthetic construct
  source 1..216: note = Heterodimeric Fc Backbone 15 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 514 - protein, 216 aa, synthetic construct
  source 1..216: note = Heterodimeric Fc Backbone 15 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 515 - protein, 216 aa, synthetic construct
  source 1..216: note = Heterodimeric Fc Backbone 16 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 516 - protein, 216 aa, synthetic construct
  source 1..216: note = Heterodimeric Fc Backbone 16 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 517 - protein, 216 aa, synthetic construct
  source 1..216: note = Heterodimeric Fc Backbone 17 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 518 - protein, 216 aa, synthetic construct
  source 1..216: note = Heterodimeric Fc Backbone 17 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 519 - protein, 216 aa, synthetic construct; identical to SEQ ID NO: 485
  source 1..216: note = 2 + 1 mAb-scFv Heterodimeric Fc Backbone monomer 1

SEQ ID NO: 520 - protein, 215 aa, synthetic construct
  source 1..215: note = 2 + 1 mAb-scFv Heterodimeric Fc Backbone monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 521 - protein, 217 aa, synthetic construct; identical to SEQ ID NO: 509
  source 1..217: note = 2 + 1 mAb-scFv Heterodimeric Fc Backbone monomer 1

SEQ ID NO: 522 - protein, 216 aa, synthetic construct
  source 1..216: note = 2 + 1 mAb-scFv Heterodimeric Fc Backbone monomer 2
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 523 - protein, 98 aa, synthetic construct
  source 1..98: note = IgG1 CH1(+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 524 - protein, 98 aa, synthetic construct
  source 1..98: note = IgG1 CH1(-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKV

SEQ ID NO: 525 - protein, 98 aa, synthetic construct
  source 1..98: note = IgG2 CH1(+)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 526 - protein, 98 aa, synthetic construct
  source 1..98: note = IgG2 CH1(-)
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTV

SEQ ID NO: 527 - protein, 98 aa, synthetic construct
  source 1..98: note = IgG4 CH1(+)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 528 - protein, 98 aa, synthetic construct
  source 1..98: note = IgG4 CH1(-)
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRV

SEQ ID NO: 529 - protein, 15 aa, synthetic construct
  source 1..15: note = IgG1 hinge
EPKSCDKTHTCPPCP

SEQ ID NO: 530 - protein, 12 aa, synthetic construct
  source 1..12: note = IgG2 hinge
ERKCCVECPPCP

SEQ ID NO: 531 - protein, 12 aa, synthetic construct
  source 1..12: note = IgG4 hinge
ESKYGPPCPSCP

SEQ ID NO: 532 - protein, 107 aa, synthetic construct [sequence omitted]

SEQ ID NO: 533 - protein, 106 aa, synthetic construct
  source 1..106: note = Light Chain Constant Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 534 - protein, 450 aa, synthetic construct
  source 1..450: note = XENP27181 Heavy Chain
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 535 - protein, 218 aa, synthetic construct
  source 1..218: note = XENP27181 Light Chain
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 536 - protein, 446 aa, synthetic construct
  source 1..446: note = XENP29154 Heavy Chain - TGN1412 HC
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 537 - protein, 214 aa, synthetic construct
  source 1..214: note = XENP29154 Light Chain - TGN1412 LC
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 538 - protein, 449 aa, synthetic construct
  source 1..449: note = XENP31600 Chain 1 - D7[PSMA]_H0_IgG1_pI(-)_Isosteric_A_/PVA_/S267K/L368D/K370S
QVQLQQSGAELVEPGASVKLSCKASGYTFTYFDINWLRQRPEQGLEWIGGISPGDGNTNYNENFKGKATLTIDKSSTTAYIQLSRLTSEDSAVYFCARDGNFPYYAMDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 539 - protein, 485 aa, synthetic construct
  source 1..485: note = XENP31600 Chain 2 - [CD3]_H1.30_L1.47_scFv(GKPGS)4_ Fc(222)_IgG1_C220S/PVA_/S267K/S364K/E357Q
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 218 aa, synthetic construct
  source 1..218: note = XENP31600 Chain 3 - D7_L0 Light Chain
DIELTQSPLSLPVILGDQASISCRSSQSLVHSNGNTYLHWFLQKPGQSPKLLIYTVSNRFSGVPDRFSGSGSGTDFTLKISRVEAEDLGVYFCSQSTHVPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 444 aa, synthetic construct
  source 1..444: note = XENP32220 Chain 1 - PSMA-H_H1_IgG1_pI(-)_Isosteric_A_/PVA_/S267K/L368D/K370S
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 542 - protein, 717 aa, synthetic construct
  source 1..717: note = XENP32220 Chain 2 - PSMA-H_H1_(G4S)_[CD3]_L1.47_H1. 32_scFv(GKPGS)4_Fc(222)_IgG1_C220S/PVA_/S267K/S364K/E357Q
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 214 aa, synthetic construct
  source 1..214: note = XENP32220 Chain 3 - PSMA-H_L1.24 Light Chain
DIVMTQSPDSLAVSLGERATLSCRASQDVGTAVDWYQQKPDQSPKLLIYYASTRHTGVPDRFTGSGSGTDFTLTISSLQAEDVAVYFCQQYNSYPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 544 - protein, 444 aa, synthetic construct; identical to SEQ ID NO: 541
  source 1..444: note = XENP33063 Chain 1 - PSMA-H_H1_IgG1_pI(-)_Isosteric_A_/PVA_/S267K/L368D/K370S

SEQ ID NO: 545 - protein, 713 aa, synthetic construct
  source 1..713: note = XENP33063 Chain 2 - PSMA-H_H1_(G4S)_[CD3]_L1.47_H1. 89_scFv(GKPGS)4_Fc(216)_IgG1_C220S/PVA_/S267K/S364K/E357Q
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 546 - protein, 214 aa, synthetic construct
  source 1..214: note = XENP33063 Chain 3 - PSMA-H_L1.58 Light Chain
DIVMTQSPDSLAVSLGERATLSCRASQDVGTAVDWYQQKPDQSPKLLIYWASTRHTGVPDRFTGSGSGTDFTLTISSLQAEDVAVYFCQQYQSYPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 547 - protein, 479 aa, synthetic construct
  source 1..479: note = 1391 PSMA x CD3 Chain 1
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 548 - protein, 450 aa, synthetic construct
  source 1..450: note = 1391 PSMA x CD3 Chain 2
EVQLVESGGGLVKPGGSLRLSCVASGFTFSFYSMNWVRQAPGKGLDWVSSISSSGNYIYYADSVKGRFTISRDNAKNSLHLHMNSLKAEDTAMYFCARSYSGSYDAFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 549 - protein, 215 aa, synthetic construct
  source 1..215: note = 1391 PSMA x CD3 Chain 3
EIVMTQSPGTLSLSPGERATLSCRASQSVSSSFLAWYQQKPGQAPRLLISGASSRATGIPDRFSVSGSGTDFTLTISRLEPEDFAVYYCQQYGVSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 550 - protein, 475 aa, synthetic construct
  source 1..475: note = 1508 PSMA x CD3 Chain 1
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 551 - protein, 451 aa, synthetic construct
  source 1..451: note = 1508 PSMA x CD3 Chain 2
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 552 - protein, 216 aa, synthetic construct
  source 1..216: note = 1508 PSMA x CD3 Chain 3
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 553 - protein, 449 aa, synthetic construct; identical to SEQ ID NO: 538
  source 1..449: note = XENP37900 Chain 1 - D7[PSMA]_H0_IgG1_pI(-)_Isosteric_A_/PVA_/S267K/L368D/K370S

SEQ ID NO: 554 - protein, 479 aa, synthetic construct
  source 1..479: note = XENP37900 Chain 2 - 1A7[CD28]_H1.14_L1_scFv(GKPGS)4 _Fc(216)_IgG1_C220S/PVA_/S267K/S364K/E357Q
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 555 - protein, 218 aa, synthetic construct; identical to SEQ ID NO: 540
  source 1..218: note = XENP37900 Chain 3 - D7[PSMA]_L0 Light Chain

SEQ ID NO: 556 - protein, 449 aa, synthetic construct; identical to SEQ ID NO: 538
  source 1..449: note = XENP37901 Chain 1 - D7[PSMA]_H0_IgG1_pI(-)_Isosteric_A_/PVA_/S267K/L368D/K370S

SEQ ID NO: 557 - protein, 479 aa, synthetic construct
  source 1..479: note = XENP37901 Chain 2 - 1A7[CD28]_H1.14_L1.71_scFv(GKPG S)4_Fc(216)_IgG1_C220S/PVA_/S267K/S364K/E357Q
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 558 - protein, 218 aa, synthetic construct; identical to SEQ ID NO: 540
  source 1..218: note = XENP37901 Chain 3 - D7[PSMA]_L0 Light Chain

SEQ ID NO: 559 - protein, 444 aa, synthetic construct; identical to SEQ ID NO: 541
  source 1..444: note = XENP37902 Chain 1 - PSMA-H[PSMA]_H1_IgG1_pI(-)_Isos teric_A_/PVA_/S267K/L368D/K370S

SEQ ID NO: 560 - protein, 214 aa, synthetic construct
  source 1..214: note = XENP37902 Chain 3 - PSMA-H[PSMA]_L1 Light Chain
DIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 561 - protein, 444 aa, synthetic construct; identical to SEQ ID NO: 541
  source 1..444: note = XENP37903 Chain 1 - PSMA-H[PSMA]_H1_IgG1_pI(-)_Isos teric_A_/PVA_/S267K/L368D/K370S

SEQ ID NO: 562 - protein, 214 aa, synthetic construct; identical to SEQ ID NO: 560
  source 1..214: note = XENP37903 Chain 3 - PSMA-H[PSMA]_L1 Light Chain

SEQ ID NO: 563 - protein, 449 aa, synthetic construct
  source 1..449: note = XENP38931 Chain 1 - P72_A10[PSMA]_H0_IgG1_pI(-)_Iso steric_A_/PVA_/S267K/L368D/K370S
QVQLVESGGGVVQPGRSLRLSCAASGFTFSSYNMNWVRQAPGKGLEWVAIIYYDGSNKYYADSVKGRFTISRDISKNTLYLQMNSLRAEDTAVYYCARERGRDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 564 - protein, 479 aa, synthetic construct
  source 1..479: note = XENP38931 Chain 2 - 1A7[CD28]_H1_L1.71_scFv(GKPGS)4 _Fc(216)_IgG1_C220S/PVA_/S267K/S364K/E357Q
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 565 - protein, 213 aa, synthetic construct; identical to SEQ ID NO: 344
  source 1..213: note = XENP38931 Chain 3 - P72_A10[PSMA]_L0 Light Chain

SEQ ID NO: 566 - protein, 455 aa, synthetic construct
  source 1..455: note = XENP38932 Chain 1 - P72_D01[PSMA]_H0_IgG1_pI(-)_Iso steric_A_/PVA_/S267K/L368D/K370S
EVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCARDGVGADYGDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 567 - protein, 479 aa, synthetic construct; identical to SEQ ID NO: 564
  source 1..479: note = XENP38932 Chain 2 - 1A7[CD28]_H1_L1.71_scFv(GKPGS)4 _Fc(216)_IgG1_C220S/PVA_/S267K/S364K/E357Q

SEQ ID NO: 568 - protein, 216 aa, synthetic construct
  source 1..216: note = XENP38932 Chain 3 - P72_D01[PSMA]_L0 Light Chain
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSYTYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 569 - protein, 454 aa, synthetic construct
  source 1..454: note = XENP38933 Chain 1 - P72_E07[PSMA]_H0_IgG1_pI(-)_Iso steric_A_/PVA_/S267K/L368D/K370S
EVQLVESGGGVVQPGRSLRLSCAASGFTFITYGMHWVRQAPGKGLEWVAVVSFDESNKYYADSVKGRFTISRDNSKNTLYLQMNSLRAEDTAVYYCARALRDGNNWDYFNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 570 - protein, 479 aa, synthetic construct; identical to SEQ ID NO: 564
  source 1..479: note = XENP38933 Chain 2 - 1A7[CD28]_H1_L1.71_scFv(GKPGS)4 _Fc(216)_IgG1_C220S/PVA_/S267K/S364K/E357Q

SEQ ID NO: 571 - protein, 216 aa, synthetic construct
  source 1..216: note = XENP38933 Chain 3 - P72_E07[PSMA]_L0 Light Chain
QSVLTQPPSASGTPGQGVTISCSGSSSNIGSNTVNWFQQLPGTAPKLLIYSDNQRPSGVPDRFSGSKSGTSASLAISGLQSEDEADYYCAAWDDSLNGYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 572 - protein, 447 aa, synthetic construct
  source 1..447: note = XENP38934 Chain 1 - P70_F02[PSMA]_H0_IgG1_pI(-)_Iso steric_A_/PVA_/S267K/L368D/K370S
EVQLLESGPGLVKPSETLSLTCTVSGGSIISYYWSWIRQPAGKGLEWIGRIYSSGSTNYNPSLKSRVTMSVDTSKNQFSLKLSSVTAADTAVYYCAKVGVWPGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 573 - protein, 479 aa, synthetic construct; identical to SEQ ID NO: 564
  source 1..479: note = XENP38934 Chain 2 - 1A7[CD28]_H1_L1.71_scFv(GKPGS)4 _Fc(216)_IgG1_C220S/PVA_/S267K/S364K/E357Q

SEQ ID NO: 574 - protein, 216 aa, synthetic construct
  source 1..216: note = XENP38934 Chain 3 - P70_F02[PSMA]_L0 Light Chain
QSVLTQPPSASGTPGQRVTISCSGSSSNIGSNTVNWYQQLPGTAPKLLIYSSNQRPSGVPDRFSGSKSGTSASLAISGLQSEDEADYYCAAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 575 - protein, 451 aa, synthetic construct
  source 1..451: note = XENP38935 Chain 1 - P72_A11[PSMA]_H0_IgG1_pI(-)_Iso steric_A_/PVA_/S267K/L368D/K370S
QVQLQESGGDVVQPGRSLRLSCAASGFSFSGYGLHWVRQAPGRGLEWVTLISYDGSNKYYADSVKGRFTISRDNSKNTLYLQMNSLRAEDTAVYYCAKTTVSDPYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 576 - protein, 479 aa, synthetic construct; identical to SEQ ID NO: 564
  source 1..479: note = XENP38935 Chain 2 - 1A7[CD28]_H1_L1.71_scFv(GKPGS)4 _Fc(216)_IgG1_C220S/PVA_/S267K/S364K/E357Q

SEQ ID NO: 577 - protein, 214 aa, synthetic construct
  source 1..214: note = XENP38935 Chain 3 - P72_A11[PSMA]_L0 Light Chain
SYELTQPPSVSVAPGQTARITCGGNNIGSKSVHWYQQKPGQAPVLVVYDDSDRPSGIPERFSGTNSGNTATLTISRAEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 578 - protein, 449 aa, synthetic construct
  source 1..449: note = XENP38936 Chain 1 - P72_A10V2[PSMA]_H0_IgG1_pI(-)_I sosteric_A_/PVA_/S267K/L368D/K370S
QVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 579 - protein, 479 aa, synthetic construct; identical to SEQ ID NO: 564
  source 1..479: note = XENP38936 Chain 2 - 1A7[CD28]_H1_L1.71_scFv(GKPGS)4 _Fc(216)_IgG1_C220S/PVA_/S267K/S364K/E357Q

SEQ ID NO: 580 - protein, 213 aa, synthetic construct; identical to SEQ ID NO: 344
  source 1..213: note = XENP38936 Chain 3 - P72_A10V2[PSMA]_L0 Light Chain

SEQ ID NO: 581 - protein, 455 aa, synthetic construct
  source 1..455: note = XENP38937 Chain 1 - P72_D01V2[PSMA]_H0_IgG1_pI(-)_I sosteric_A_/PVA_/S267K/L368D/K370S
EVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCAREGVGADYGDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 582 - protein, 479 aa, synthetic construct; identical to SEQ ID NO: 564
  source 1..479: note = XENP38937 Chain 2 - 1A7[CD28]_H1_L1.71_scFv(GKPGS)4 _Fc(216)_IgG1_C220S/PVA_/S267K/S364K/E357Q

SEQ ID NO: 583 - protein, 216 aa, synthetic construct; identical to SEQ ID NO: 568
  source 1..216: note = XENP38937 Chain 3 - P72_D01V2[PSMA]_L0 Light Chain

SEQ ID NO: 584 - protein, 451 aa, synthetic construct
  source 1..451: note = XENP38938 Chain 1 - P72_F07[PSMA]_H0_IgG1_pI(-)_Iso steric_A_/PVA_/S267K/L368D/K370S
EVQLVESGGGVVQPGRSLRLSCAASGFTFSSYGMNWVRQAPGKGLEWVAVTSYDGSNKYYADSVKGRFTISRDISKNTLYLQMSSLRAEDTAVYYCARDPYSSSWNGAFDIWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 585 - protein, 479 aa, synthetic construct; identical to SEQ ID NO: 564
  source 1..479: note = XENP38938 Chain 2 - 1A7[CD28]_H1_L1.71_scFv(GKPGS)4 _Fc(216)_IgG1_C220S/PVA_/S267K/S364K/E357Q

SEQ ID NO: 586 - protein, 214 aa, synthetic construct
  source 1..214: note = XENP38938 Chain 3 - P72_F07[PSMA]_L0 Light Chain
SYELTQPPSVSVAPGQTARITCGGNNIGSKSVHWYQQKPGQAPVLVVYDDSDRPSGIPERFSGSNSGNTATLTISRVEAGDEADYYCQVWDSSTDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 587 - protein, 453 aa, synthetic construct
  source 1..453: note = XENP38939 Chain 1 - P72_G02[PSMA]_H0_IgG1_pI(-)_Iso steric_A_/PVA_/S267K/L368D/K370S
EVQLVESGGGVVQPGRSLRLSCAASGFSFSGYGMHWVRQAPGKGLEWVAVISYDGSNKYYADSVKGRFTISRDNSKNTLYLQMNSLRVEDTAVYYCARDRIWGSRGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 588 - protein, 479 aa, synthetic construct; identical to SEQ ID NO: 564
  source 1..479: note = XENP38939 Chain 2 - 1A7[CD28]_H1_L1.71_scFv(GKPGS)4 _Fc(216)_IgG1_C220S/PVA_/S267K/S364K/E357Q

SEQ ID NO: 589 - protein, 216 aa, synthetic construct
  source 1..216: note = XENP38939 Chain 3 - P72_G02[PSMA]_L0 Light Chain
QSALTQPASVSGSPGQSITISCTGASSDVGGYNYVSWYQQHPGKAPKLMIYEVSNRPSGVSNRFSGSKSGNTASLTISGLQAEDEADYYCSSYTITSTLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 590 - protein, 451 aa, synthetic construct
  source 1..451: note = XENP38940 Chain 1 - P75_F01[PSMA]_H0_IgG1_pI(-)_Iso steric_A_/PVA_/S267K/L368D/K370S
QVQLQESGGGVVQPGRSLRLSCAASGFTFSTYGMHWVRQAPGKGLEWVAFISYDGSNKYYADSVKGRFTISRDNSKHTLYLQMNSLRAEDTAVYYCAGRDNLRFLEWFMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 591 - protein, 479 aa, synthetic construct; identical to SEQ ID NO: 564
  source 1..479: note = XENP38940 Chain 2 - 1A7[CD28]_H1_L1.71_scFv(GKPGS)4 _Fc(216)_IgG1_C220S/PVA_/S267K/S364K/E357Q

SEQ ID NO: 592 - protein, 215 aa, synthetic construct
  source 1..215: note = XENP38940 Chain 3 - P75_F01[PSMA]_L0 Light Chain
EIVLTQSPGTLSVSPGERATLSCRASQSVRSNLAWYQQKPGQAPRLLIYGASTRATGIPARFSGSGSGTEFTLTISSLQSEDFAVYYCHQYNDWPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 593 - protein, 451 aa, synthetic construct
  source 1..451: note = XENP38941 Chain 1 - P72_F07V2[PSMA]_H0_IgG1_pI(-)_I sosteric_A_/PVA_/S267K/L368D/K370S
EVQLVESGGGVVQPGRSLRLSCAASGFTFSSYGMNWVRQAPGKGLEWVAVTSYDESNKYYADSVKGRFTISRDISKNTLYLQMSSLRAEDTAVYYCARDPYSSSWNGAFDIWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 594 - protein, 479 aa, synthetic construct; identical to SEQ ID NO: 564
  source 1..479: note = XENP38941 Chain 2 - 1A7[CD28]_H1_L1.71_scFv(GKPGS)4 _Fc(216)_IgG1_C220S/PVA_/S267K/S364K/E357Q

SEQ ID NO: 595 - protein, 214 aa, synthetic construct; identical to SEQ ID NO: 586
  source 1..214: note = XENP38941 Chain 3 - P72_F07V2[PSMA]_L0 Light Chain

SEQ ID NO: 596 - protein, 453 aa, synthetic construct
  source 1..453: note = XENP38942 Chain 1 - P72_G02V2[PSMA]_H0_IgG1_pI(-)_I sosteric_A_/PVA_/S267K/L368D/K370S
EVQLVESGGGVVQPGRSLRLSCAASGFSFSGYGMHWVRQAPGKGLEWVAVISYDESNKYYADSVKGRFTISRDNSKNTLYLQMNSLRVEDTAVYYCARDRIWGSRGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 597 - protein, 479 aa, synthetic construct; identical to SEQ ID NO: 564
  source 1..479: note = XENP38942 Chain 2 - 1A7[CD28]_H1_L1.71_scFv(GKPGS)4 _Fc(216)_IgG1_C220S/PVA_/S267K/S364K/E357Q

SEQ ID NO: 598 - protein, 216 aa, synthetic construct; identical to SEQ ID NO: 589
  source 1..216: note = XENP38942 Chain 3 - P72_G02V2[PSMA]_L0 Light Chain

SEQ ID NO: 599 - protein, 451 aa, synthetic construct
  source 1..451: note = XENP38943 Chain 1 - P75_F01V2[PSMA]_H0_IgG1_pI(-)_I sosteric_A_/PVA_/S267K/L368D/K370S
QVQLQESGGGVVQPGRSLRLSCAASGFTFSTYGMHWVRQAPGKGLEWVAFISYDESNKYYADSVKGRFTISRDNSKHTLYLQMNSLRAEDTAVYYCAGRDNLRFLEWFMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 600 - protein, 479 aa, synthetic construct; identical to SEQ ID NO: 564
  source 1..479: note = XENP38943 Chain 2 - 1A7[CD28]_H1_L1.71_scFv(GKPGS)4 _Fc(216)_IgG1_C220S/PVA_/S267K/S364K/E357Q

SEQ ID NO: 601 - protein, 215 aa, synthetic construct; identical to SEQ ID NO: 592
  source 1..215: note = XENP38943 Chain 3 - P75_F01V2[PSMA]_L0 Light Chain

SEQ ID NO: 602 - protein, 450 aa, synthetic construct
  source 1..450: note = XENP38944 Chain 1 - PSMB896-HC-G100A[PSMA]_H0_IgG1_ pI(-)_Isosteric_A_/PVA_/S267K/L368D/K370S
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 603 - protein, 479 aa, synthetic construct; identical to SEQ ID NO: 564
  source 1..479: note = XENP38944 Chain 2 - 1A7[CD28]_H1_L1.71_scFv(GKPGS)4 _Fc(216)_IgG1_C220S/PVA_/S267K/S364K/E357Q

SEQ ID NO: 604 - protein, 216 aa, synthetic construct; identical to SEQ ID NO: 552
  source 1..216: note = XENP38944 Chain 3 - PSMB896-HC-G100A[PSMA]_L0 Light Chain

SEQ ID NO: 605 - protein, 449 aa, synthetic construct
  source 1..449: note = XENP38945 Chain 1 - 011A11[PSMA]_H0_IgG1_pI(-)_Isos teric_A_/PVA_/S267K/L368D/K370S
EVQLVESGGGLVKPGGSLRLSCVASGFTFSFYSMNWVRQAPGKGLDWVSSISSSGNYIYYADSVKGRFTISRDNAKNSLHLHMNSLKAEDTAMYFCARSYSGSYDAFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 606 - protein, 479 aa, synthetic construct; identical to SEQ ID NO: 564
  source 1..479: note = XENP38945 Chain 2 - 1A7[CD28]_H1_L1.71_scFv(GKPGS)4 _Fc(216)_IgG1_C220S/PVA_/S267K/S364K/E357Q

SEQ ID NO: 607 - protein, 215 aa, synthetic construct; identical to SEQ ID NO: 549
  source 1..215: note = XENP38945 Chain 3 - 011A11[PSMA]_L0 Light Chain

SEQ ID NO: 608 - protein, 449 aa, synthetic construct; identical to SEQ ID NO: 563
  source 1..449: note = XENP39211 Chain 1 - P72_A10[PSMA]_H0_IgG1_pI(-)_Iso steric_A_/PVA_/S267K/L368D/K370S

SEQ ID NO: 609 - protein, 479 aa, synthetic construct
  source 1..479: note = XENP39211 Chain 2 - 1A7[CD28]_H1L1_scFv(GKPGS)4_Fc( 216)_IgG1_C220S/PVA_/S267K/S364K/E357Q
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 610 - protein, 213 aa, synthetic construct; identical to SEQ ID NO: 344
  source 1..213: note = XENP39211 Chain 3 - P72_A10[PSMA]_L0 Light Chain

SEQ ID NO: 611 - protein, 449 aa, synthetic construct; identical to SEQ ID NO: 563
  source 1..449: note = XENP39212 Chain 1 - P72_A10[PSMA]_H0_IgG1_pI(-)_Iso steric_A_/PVA_/S267K/L368D/K370S

SEQ ID NO: 612 - protein, 479 aa, synthetic construct; identical to SEQ ID NO: 554
  source 1..479: note = XENP39212 Chain 2 - 1A7[CD28]_H1.14_L1_scFv(GKPGS)4 _Fc(216)_IgG1_C220S/PVA_/S267K/S364K/E357Q

SEQ ID NO: 613 - protein, 213 aa, synthetic construct; identical to SEQ ID NO: 344
  source 1..213: note = XENP39212 Chain 3 - P72_A10[PSMA]_L0 Light Chain

SEQ ID NO: 614 - protein, 449 aa, synthetic construct; identical to SEQ ID NO: 563
  source 1..449: note = XENP39213 Chain 1 - P72_A10[PSMA]_H0_IgG1_pI(-)_Iso steric_A_/PVA_/S267K/L368D/K370S

SEQ ID NO: 615 - protein, 479 aa, synthetic construct
  source 1..479: note = XENP39213 Chain 2 - 1A7[CD28]_H1.1_L1.71_scFv(GKPGS )4_Fc(216)_IgG1_C220S/PVA_/S267K/S364K/E357Q
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 616 - protein, 213 aa, synthetic construct; identical to SEQ ID NO: 344
  source 1..213: note = XENP39213 Chain 3 - P72_A10[PSMA]_L0 Light Chain

SEQ ID NO: 617 - protein, 449 aa, synthetic construct; identical to SEQ ID NO: 563
  source 1..449: note = XENP39214 Chain 1 - P72_A10[PSMA]_H0_IgG1_pI(-)_Iso steric_A_/PVA_/S267K/L368D/K370S

SEQ ID NO: 618 - protein, 479 aa, synthetic construct; identical to SEQ ID NO: 557
  source 1..479: note = XENP39214 Chain 2 - 1A7[CD28]_H1.14_L1.71_scFv(GKPG S)4_Fc(216)_IgG1_C220S/PVA_/S267K/S364K/E357Q

SEQ ID NO: 619 - protein, 213 aa, synthetic construct; identical to SEQ ID NO: 344
  source 1..213: note = XENP39214 Chain 3 - P72_A10[PSMA]_L0 Light Chain

SEQ ID NO: 620 - protein, 455 aa, synthetic construct; identical to SEQ ID NO: 566
  source 1..455: note = XENP39215 Chain 1 - P72_D01[PSMA]_H0_IgG1_pI(-)_Iso steric_A_/PVA_/S267K/L368D/K370S

SEQ ID NO: 621 - protein, 479 aa, synthetic construct; identical to SEQ ID NO: 609
  source 1..479: note = XENP39215 Chain 2 - 1A7[CD28]_H1L1_scFv(GKPGS)4_Fc( 216)_IgG1_C220S/PVA_/S267K/S364K/E357Q

SEQ ID NO: 622 - protein, 216 aa, synthetic construct; identical to SEQ ID NO: 568
  source 1..216: note = XENP39215 Chain 3 - P72_D01[PSMA]_L0 Light Chain

SEQ ID NO: 623 - protein, 455 aa, synthetic construct; identical to SEQ ID NO: 566
  source 1..455: note = XENP39216 Chain 1 - P72_D01[PSMA]_H0_IgG1_pI(-)_Iso steric_A_/PVA_/S267K/L368D/K370S

SEQ ID NO: 624 - protein, 479 aa, synthetic construct; identical to SEQ ID NO: 554
  source 1..479: note = XENP39216 Chain 2 - 1A7[CD28]_H1.14_L1_scFv(GKPGS)4 _Fc(216)_IgG1_C220S/PVA_/S267K/S364K/E357Q

SEQ ID NO: 625 - protein, 216 aa, synthetic construct; identical to SEQ ID NO: 568
  source 1..216: note = XENP39216 Chain 3 - P72_D01[PSMA]_L0 Light Chain

SEQ ID NO: 626 - protein, 455 aa, synthetic construct; identical to SEQ ID NO: 566
  source 1..455: note = XENP39217 Chain 1 - P72_D01[PSMA]_H0_IgG1_pI(-)_Iso steric_A_/PVA_/S267K/L368D/K370S

SEQ ID NO: 627 - protein, 479 aa, synthetic construct; identical to SEQ ID NO: 615
  source 1..479: note = XENP39217 Chain 2 - 1A7[CD28]_H1.1_L1.71_scFv(GKPGS )4_Fc(216)_IgG1_C220S/PVA_/S267K/S364K/E357Q

SEQ ID NO: 628 - protein, 216 aa, synthetic construct; identical to SEQ ID NO: 568
  source 1..216: note = XENP39217 Chain 3 - P72_D01[PSMA]_L0 Light Chain

SEQ ID NO: 629 - protein, 455 aa, synthetic construct; identical to SEQ ID NO: 566
  source 1..455: note = XENP39218 Chain 1 - P72_D01[PSMA]_H0_IgG1_pI(-)_Iso steric_A_/PVA_/S267K/L368D/K370S

SEQ ID NO: 630 - protein, 479 aa, synthetic construct; identical to SEQ ID NO: 557
  source 1..479: note = XENP39218 Chain 2 - 1A7[CD28]_H1.14_L1.71_scFv(GKPG S)4_Fc(216)_IgG1_C220S/PVA_/S267K/S364K/E357Q

SEQ ID NO: 631 - protein, 216 aa, synthetic construct; identical to SEQ ID NO: 568
  source 1..216: note = XENP39218 Chain 3 - P72_D01[PSMA]_L0 Light Chain

SEQ ID NO: 632 - protein, 454 aa, synthetic construct; identical to SEQ ID NO: 569
  source 1..454: note = XENP39219 Chain 1 - P72_E07[PSMA]_H0_IgG1_pI(-)_Iso steric_A_/PVA_/S267K/L368D/K370S

SEQ ID NO: 633 - protein, 479 aa, synthetic construct; identical to SEQ ID NO: 609
  source 1..479: note = XENP39219 Chain 2 - 1A7[CD28]_H1L1_scFv(GKPGS)4_Fc( 216)_IgG1_C220S/PVA_/S267K/S364K/E357Q

SEQ ID NO: 634 - protein, 216 aa, synthetic construct; identical to SEQ ID NO: 571
  source 1..216: note = XENP39219 Chain 3 - P72_E07[PSMA]_L0 Light Chain

SEQ ID NO: 635 - protein, 454 aa, synthetic construct; identical to SEQ ID NO: 569
  source 1..454: note = XENP39220 Chain 1 - P72_E07[PSMA]_H0_IgG1_pI(-)_Iso steric_A_/PVA_/S267K/L368D/K370S

SEQ ID NO: 636 - protein, 479 aa, synthetic construct; identical to SEQ ID NO: 554
  source 1..479: note = XENP39220 Chain 2 - 1A7[CD28]_H1.14_L1_scFv(GKPGS)4 _Fc(216)_IgG1_C220S/PVA_/S267K/S364K/E357Q

SEQ ID NO: 637 - protein, 216 aa, synthetic construct; identical to SEQ ID NO: 571
  source 1..216: note = XENP39220 Chain 3 - P72_E07[PSMA]_L0 Light Chain

SEQ ID NO: 638 - protein, 454 aa, synthetic construct; identical to SEQ ID NO: 569
  source 1..454: note = XENP39221 Chain 1 - P72_E07[PSMA]_H0_IgG1_pI(-)_Iso steric_A_/PVA_/S267K/L368D/K370S

SEQ ID NO: 639 - protein, 479 aa, synthetic construct; identical to SEQ ID NO: 615
  source 1..479: note = XENP39221 Chain 2 - 1A7[CD28]_H1.1_L1.71_scFv(GKPGS )4_Fc(216)_IgG1_C220S/PVA_/S267K/S364K/E357Q

SEQ ID NO: 640 - protein, 216 aa, synthetic construct; identical to SEQ ID NO: 571
  source 1..216: note = XENP39221 Chain 3 - P72_E07[PSMA]_L0 Light Chain

SEQ ID NO: 641 - protein, 454 aa, synthetic construct; identical to SEQ ID NO: 569
  source 1..454: note = XENP39222 Chain 1 - P72_E07[PSMA]_H0_IgG1_pI(-)_Iso steric_A_/PVA_/S267K/L368D/K370S

SEQ ID NO: 642 - protein, 479 aa, synthetic construct; identical to SEQ ID NO: 557
  source 1..479: note = XENP39222 Chain 2 - 1A7[CD28]_H1.14_L1.71_scFv(GKPG S)4_Fc(216)_IgG1_C220S/PVA_/S267K/S364K/E357Q

SEQ ID NO: 643 - protein, 216 aa, synthetic construct; identical to SEQ ID NO: 571
  source 1..216: note = XENP39222 Chain 3 - P72_E07[PSMA]_L0 Light Chain

SEQ ID NO: 644 - protein, 447 aa, synthetic construct; identical to SEQ ID NO: 572
  source 1..447: note = XENP39223 Chain 1 - P70_F02[PSMA]_H0_IgG1_pI(-)_Iso steric_A_/PVA_/S267K/L368D/K370S

SEQ ID NO: 645 - protein, 479 aa, synthetic construct; identical to SEQ ID NO: 609
  source 1..479: note = XENP39223 Chain 2 - 1A7[CD28]_H1L1_scFv(GKPGS)4_Fc( 216)_IgG1_C220S/PVA_/S267K/S364K/E357Q

SEQ ID NO: 646 - protein, 216 aa, synthetic construct; identical to SEQ ID NO: 574
  source 1..216: note = XENP39223 Chain 3 - P70_F02[PSMA]_L0 Light Chain

SEQ ID NO: 647 - protein, 447 aa, synthetic construct; identical to SEQ ID NO: 572
  source 1..447: note = XENP39224 Chain 1 - P70_F02[PSMA]_H0_IgG1_pI(-)_Iso steric_A_/PVA_/S267K/L368D/K370S

SEQ ID NO: 648 - protein, 479 aa, synthetic construct; identical to SEQ ID NO: 554
  source 1..479: note = XENP39224 Chain 2 - 1A7[CD28]_H1.14_L1_scFv(GKPGS)4 _Fc(216)_IgG1_C220S/PVA_/S267K/S364K/E357Q

SEQ ID NO: 649 - protein, 216 aa, synthetic construct; identical to SEQ ID NO: 574
  source 1..216: note = XENP39224 Chain 3 - P70_F02[PSMA]_L0 Light Chain

SEQ ID NO: 650 - protein, 447 aa, synthetic construct; identical to SEQ ID NO: 572
  source 1..447: note = XENP39225 Chain 1 - P70_F02[PSMA]_H0_IgG1_pI(-)_Iso steric_A_/PVA_/S267K/L368D/K370S

SEQ ID NO: 651 - protein, 479 aa, synthetic construct; identical to SEQ ID NO: 615
  source 1..479: note = XENP39225 Chain 2 - 1A7[CD28]_H1.1_L1.71_scFv(GKPGS )4_Fc(216)_IgG1_C220S/PVA_/S267K/S364K/E357Q

SEQ ID NO: 652 - protein, 216 aa, synthetic construct; identical to SEQ ID NO: 574
  source 1..216: note = XENP39225 Chain 3 - P70_F02[PSMA]_L0 Light Chain

SEQ ID NO: 653 - protein, 447 aa, synthetic construct; identical to SEQ ID NO: 572
  source 1..447: note = XENP39226 Chain 1 - P70_F02[PSMA]_H0_IgG1_pI(-)_Iso steric_A_/PVA_/S267K/L368D/K370S

SEQ ID NO: 654 - protein, 479 aa, synthetic construct; identical to SEQ ID NO: 557
  source 1..479: note = XENP39226 Chain 2 - 1A7[CD28]_H1.14_L1.71_scFv(GKPG S)4_Fc(216)_IgG1_C220S/PVA_/S267K/S364K/E357Q

SEQ ID NO: 655 - protein, 216 aa, synthetic construct; identical to SEQ ID NO: 574
  source 1..216: note = XENP39226 Chain 3 - P70_F02[PSMA]_L0 Light Chain

SEQ ID NO: 656 - protein, 451 aa, synthetic construct; identical to SEQ ID NO: 575
  source 1..451: note = XENP39227 Chain 1 - P72_A11[PSMA]_H0_IgG1_pI(-)_Iso steric_A_/PVA_/S267K/L368D/K370S

SEQ ID NO: 657 - protein, 479 aa, synthetic construct; identical to SEQ ID NO: 609
  source 1..479: note = XENP39227 Chain 2 - 1A7[CD28]_H1L1_scFv(GKPGS)4_Fc( 216)_IgG1_C220S/PVA_/S267K/S364K/E357Q

SEQ ID NO: 658 - protein, 214 aa, synthetic construct; identical to SEQ ID NO: 577
  source 1..214: note = XENP39227 Chain 3 - P72_A11[PSMA]_L0 Light Chain

SEQ ID NO: 659 - protein, 451 aa, synthetic construct; identical to SEQ ID NO: 575
  source 1..451: note = XENP39228 Chain 1 - P72_A11[PSMA]_H0_IgG1_pI(-)_Iso steric_A_/PVA_/S267K/L368D/K370S

SEQ ID NO: 660 - protein, 479 aa, synthetic construct; identical to SEQ ID NO: 554
  source 1..479: note = XENP39228 Chain 2 - 1A7[CD28]_H1.14_L1_scFv(GKPGS)4 _Fc(216)_IgG1_C220S/PVA_/S267K/S364K/E357Q

SEQ ID NO: 661 - protein, 214 aa, synthetic construct; identical to SEQ ID NO: 577
  source 1..214: note = XENP39228 Chain 3 - P72_A11[PSMA]_L0 Light Chain

SEQ ID NO: 662 - protein, 451 aa, synthetic construct; identical to SEQ ID NO: 575
  source 1..451: note = XENP39229 Chain 1 - P72_A11[PSMA]_H0_IgG1_pI(-)_Iso steric_A_/PVA_/S267K/L368D/K370S

SEQ ID NO: 663 - protein, 479 aa, synthetic construct; identical to SEQ ID NO: 615
  source 1..479: note = XENP39229 Chain 2 - 1A7[CD28]_H1.1_L1.71_scFv(GKPGS )4_Fc(216)_IgG1_C220S/PVA_/S267K/S364K/E357Q

SEQ ID NO: 664 - protein, 214 aa, synthetic construct; identical to SEQ ID NO: 577
  source 1..214: note = XENP39229 Chain 3 - P72_A11[PSMA]_L0 Light Chain

SEQ ID NO: 665 - protein, 451 aa, synthetic construct; identical to SEQ ID NO: 575
  source 1..451: note = XENP39230 Chain 1 - P72_A11[PSMA]_H0_IgG1_pI(-)_Iso steric_A_/PVA_/S267K/L368D/K370S

SEQ ID NO: 666 - protein, 479 aa, synthetic construct; identical to SEQ ID NO: 557
  source 1..479: note = XENP39230 Chain 2 - 1A7[CD28]_H1.14_L1.71_scFv(GKPG S)4_Fc(216)_IgG1_C220S/PVA_/S267K/S364K/E357Q

SEQ ID NO: 667 - protein, 214 aa, synthetic construct; identical to SEQ ID NO: 577
  source 1..214: note = XENP39230 Chain 3 - P72_A11[PSMA]_L0 Light Chain

SEQ ID NO: 668 - protein, 449 aa, synthetic construct; identical to SEQ ID NO: 578
  source 1..449: note = XENP39231 Chain 1 - P72_A10V2[PSMA]_H0_IgG1_pI(-)_I sosteric_A_/PVA_/S267K/L368D/K370S

SEQ ID NO: 669 - protein, 479 aa, synthetic construct; identical to SEQ ID NO: 609
  source 1..479: note = XENP39231 Chain 2 - 1A7[CD28]_H1L1_scFv(GKPGS)4_Fc( 216)_IgG1_C220S/PVA_/S267K/S364K/E357Q

SEQ ID NO: 670 - protein, 213 aa, synthetic construct; identical to SEQ ID NO: 344
  source 1..213: note = XENP39231 Chain 3 - P72_A10V2[PSMA]_L0 Light Chain

SEQ ID NO: 671 - protein, 449 aa, synthetic construct; identical to SEQ ID NO: 578
  source 1..449: note = XENP39232 Chain 1 - P72_A10V2[PSMA]_H0_IgG1_pI(-)_I sosteric_A_/PVA_/S267K/L368D/K370S

SEQ ID NO: 672 - protein, 479 aa, synthetic construct; identical to SEQ ID NO: 554
  source 1..479: note = XENP39232 Chain 2 - 1A7[CD28]_H1.14_L1_scFv(GKPGS)4 _Fc(216)_IgG1_C220S/PVA_/S267K/S364K/E357Q

SEQ ID NO: 673 - protein, 213 aa, synthetic construct; identical to SEQ ID NO: 344
  source 1..213: note = XENP39232 Chain 3 - P72_A10V2[PSMA]_L0 Light Chain

SEQ ID NO: 674 - protein, 449 aa, synthetic construct; identical to SEQ ID NO: 578
  source 1..449: note = XENP39233 Chain 1 - P72_A10V2[PSMA]_H0_IgG1_pI(-)_I sosteric_A_/PVA_/S267K/L368D/K370S

SEQ ID NO: 675 - protein, 479 aa, synthetic construct; identical to SEQ ID NO: 615
  source 1..479: note = XENP39233 Chain 2 - 1A7[CD28]_H1.1_L1.71_scFv(GKPGS )4_Fc(216)_IgG1_C220S/PVA_/S267K/S364K/E357Q

SEQ ID NO: 676 - protein, 213 aa, synthetic construct; identical to SEQ ID NO: 344
  source 1..213: note = XENP39233 Chain 3 - P72_A10V2[PSMA]_L0 Light Chain

SEQ ID NO: 677 - protein, 449 aa, synthetic construct; identical to SEQ ID NO: 578
  source 1..449: note = XENP39234 Chain 1 - P72_A10V2[PSMA]_H0_IgG1_pI(-)_I sosteric_A_/PVA_/S267K/L368D/K370S

SEQ ID NO: 678 - protein, 479 aa, synthetic construct; identical to SEQ ID NO: 557
  source 1..479: note = XENP39234 Chain 2 - 1A7[CD28]_H1.14_L1.71_scFv(GKPG S)4_Fc(216)_IgG1_C220S/PVA_/S267K/S364K/E357Q

SEQ ID NO: 679 - protein, 213 aa, synthetic construct; identical to SEQ ID NO: 344
  source 1..213: note = XENP39234 Chain 3 - P72_A10V2[PSMA]_L0 Light Chain

SEQ ID NO: 680 - protein, 455 aa, synthetic construct; identical to SEQ ID NO: 581
  source 1..455: note = XENP39235 Chain 1 - P72_D01V2[PSMA]_H0_IgG1_pI(-)_I sosteric_A_/PVA_/S267K/L368D/K370S

SEQ ID NO: 681 - protein, 479 aa, synthetic construct; identical to SEQ ID NO: 609
  source 1..479: note = XENP39235 Chain 2 - 1A7[CD28]_H1L1_scFv(GKPGS)4_Fc( 216)_IgG1_C220S/PVA_/S267K/S364K/E357Q

SEQ ID NO: 682 - protein, 216 aa, synthetic construct; identical to SEQ ID NO: 568
  source 1..216: note = XENP39235 Chain 3 - P72_D01V2[PSMA]_L0 Light Chain

SEQ ID NO: 683 - protein, 455 aa, synthetic construct; identical to SEQ ID NO: 581
  source 1..455: note = XENP39236 Chain 1 - P72_D01V2[PSMA]_H0_IgG1_pI(-)_I sosteric_A_/PVA_/S267K/L368D/K370S

SEQ ID NO: 684 - protein, 479 aa, synthetic construct; identical to SEQ ID NO: 554
  source 1..479: note = XENP39236 Chain 2 - 1A7[CD28]_H1.14_L1_scFv(GKPGS)4 _Fc(216)_IgG1_C220S/PVA_/S267K/S364K/E357Q

SEQ ID NO: 685 - protein, 216 aa, synthetic construct; identical to SEQ ID NO: 568
  source 1..216: note = XENP39236 Chain 3 - P72_D01V2[PSMA]_L0 Light Chain

SEQ ID NO: 686 - protein, 455 aa, synthetic construct; identical to SEQ ID NO: 581
  source 1..455: note = XENP39237 Chain 1 - P72_D01V2[PSMA]_H0_IgG1_pI(-)_I sosteric_A_/PVA_/S267K/L368D/K370S

SEQ ID NO: 687 - protein, 479 aa, synthetic construct; identical to SEQ ID NO: 615
  source 1..479: note = XENP39237 Chain 2 - 1A7[CD28]_H1.1_L1.71_scFv(GKPGS )4_Fc(216)_IgG1_C220S/PVA_/S267K/S364K/E357Q

SEQ ID NO: 688 - protein, 216 aa, synthetic construct; identical to SEQ ID NO: 568
  source 1..216: note = XENP39237 Chain 3 - P72_D01V2[PSMA]_L0 Light Chain

SEQ ID NO: 689 - protein, 455 aa, synthetic construct; identical to SEQ ID NO: 581
  source 1..455: note = XENP39238 Chain 1 - P72_D01V2[PSMA]_H0_IgG1_pI(-)_I sosteric_A_/PVA_/S267K/L368D/K370S

SEQ ID NO: 690 - protein, 479 aa, synthetic construct; identical to SEQ ID NO: 557
  source 1..479: note = XENP39238 Chain 2 - 1A7[CD28]_H1.14_L1.71_scFv(GKPG S)4_Fc(216)_IgG1_C220S/PVA_/S267K/S364K/E357Q

SEQ ID NO: 691 - protein, 216 aa, synthetic construct; identical to SEQ ID NO: 568
  source 1..216: note = XENP39238 Chain 3 - P72_D01V2[PSMA]_L0 Light Chain

SEQ ID NO: 692 - protein, 451 aa, synthetic construct; identical to SEQ ID NO: 584
  source 1..451: note = XENP39239 Chain 1 - P72_F07[PSMA]_H0_IgG1_pI(-)_Iso steric_A_/PVA_/S267K/L368D/K370S

SEQ ID NO: 693 - protein, 479 aa, synthetic construct; identical to SEQ ID NO: 609
  source 1..479: note = XENP39239 Chain 2 - 1A7[CD28]_H1L1_scFv(GKPGS)4_Fc( 216)_IgG1_C220S/PVA_/S267K/S364K/E357Q

SEQ ID NO: 694 - protein, 214 aa, synthetic construct; identical to SEQ ID NO: 586
  source 1..214: note = XENP39239 Chain 3 - P72_F07[PSMA]_L0 Light Chain

SEQ ID NO: 695 - protein, 451 aa, synthetic construct; identical to SEQ ID NO: 584
  source 1..451: note = XENP39240 Chain 1 - P72_F07[PSMA]_H0_IgG1_pI(-)_Iso steric_A_/PVA_/S267K/L368D/K370S

SEQ ID NO: 696 - protein, 479 aa, synthetic construct; identical to SEQ ID NO: 554
  source 1..479: note = XENP39240 Chain 2 - 1A7[CD28]_H1.14_L1_scFv(GKPGS)4 _Fc(216)_IgG1_C220S/PVA_/S267K/S364K/E357Q

SEQ ID NO: 697 - protein, 214 aa, synthetic construct; identical to SEQ ID NO: 586
  source 1..214: note = XENP39240 Chain 3 - P72_F07[PSMA]_L0 Light Chain

SEQ ID NO: 698 - protein, 451 aa, synthetic construct; identical to SEQ ID NO: 584
  source 1..451: note = XENP39241 Chain 1 - P72_F07[PSMA]_H0_IgG1_pI(-)_Iso steric_A_/PVA_/S267K/L368D/K370S

SEQ ID NO: 699 - protein, 479 aa, synthetic construct; identical to SEQ ID NO: 615
  source 1..479: note = XENP39241 Chain 2 - 1A7[CD28]_H1.1_L1.71_scFv(GKPGS )4_Fc(216)_IgG1_C220S/PVA_/S267K/S364K/E357Q

SEQ ID NO: 700 - protein, 214 aa, synthetic construct; identical to SEQ ID NO: 586
  source 1..214: note = XENP39241 Chain 3 - P72_F07[PSMA]_L0 Light Chain

SEQ ID NO: 701 - protein, 451 aa, synthetic construct; identical to SEQ ID NO: 584
  source 1..451: note = XENP39242 Chain 1 - P72_F07[PSMA]_H0_IgG1_pI(-)_Iso steric_A_/PVA_/S267K/L368D/K370S

SEQ ID NO: 702 - protein, 479 aa, synthetic construct; identical to SEQ ID NO: 557
  source 1..479: note = XENP39242 Chain 2 - 1A7[CD28]_H1.14_L1.71_scFv(GKPG S)4_Fc(216)_IgG1_C220S/PVA_/S267K/S364K/E357Q

SEQ ID NO: 703 - protein, 214 aa, synthetic construct; identical to SEQ ID NO: 586
  source 1..214: note = XENP39242 Chain 3 - P72_F07[PSMA]_L0 Light Chain

SEQ ID NO: 704 - protein, 453 aa, synthetic construct; identical to SEQ ID NO: 587
  source 1..453: note = XENP39243 Chain 1 - P72_G02[PSMA]_H0_IgG1_pI(-)_Iso steric_A_/PVA_/S267K/L368D/K370S

SEQ ID NO: 705 - protein, 479 aa, synthetic construct; identical to SEQ ID NO: 609
  source 1..479: note = XENP39243 Chain 2 - 1A7[CD28]_H1L1_scFv(GKPGS)4_Fc( 216)_IgG1_C220S/PVA_/S267K/S364K/E357Q

SEQ ID NO: 706 - protein, 216 aa, synthetic construct; identical to SEQ ID NO: 589
  source 1..216: note = XENP39243 Chain 3 - P72_G02[PSMA]_L0 Light Chain

SEQ ID NO: 707 - protein, 453 aa, synthetic construct; identical to SEQ ID NO: 587
  source 1..453: note = XENP39244 Chain 1 - P72_G02[PSMA]_H0_IgG1_pI(-)_Iso steric_A_/PVA_/S267K/L368D/K370S

SEQ ID NO: 708 - protein, 479 aa, synthetic construct; identical to SEQ ID NO: 554
  source 1..479: note = XENP39244 Chain 2 - 1A7[CD28]_H1.14_L1_scFv(GKPGS)4 _Fc(216)_IgG1_C220S/PVA_/S267K/S364K/E357Q

SEQ ID NO: 709 - protein, 216 aa, synthetic construct; identical to SEQ ID NO: 589
  source 1..216: note = XENP39244 Chain 3 - P72_G02[PSMA]_L0 Light Chain

SEQ ID NO: 710 - protein, 453 aa, synthetic construct; identical to SEQ ID NO: 587
  source 1..453: note = XENP39245 Chain 1 - P72_G02[PSMA]_H0_IgG1_pI(-)_Iso steric_A_/PVA_/S267K/L368D/K370S

SEQ ID NO: 711 - protein, 479 aa, synthetic construct; identical to SEQ ID NO: 615
  source 1..479: note = XENP39245 Chain 2 - 1A7[CD28]_H1.1_L1.71_scFv(GKPGS )4_Fc(216)_IgG1_C220S/PVA_/S267K/S364K/E357Q

SEQ ID NO: 712 - protein, 216 aa, synthetic construct; identical to SEQ ID NO: 589
  source 1..216: note = XENP39245 Chain 3 - P72_G02[PSMA]_L0 Light Chain

SEQ ID NO: 713 - protein, 453 aa, synthetic construct; identical to SEQ ID NO: 587
  source 1..453: note = XENP39246 Chain 1 - P72_G02[PSMA]_H0_IgG1_pI(-)_Iso steric_A_/PVA_/S267K/L368D/K370S

SEQ ID NO: 714 - protein, 479 aa, synthetic construct; identical to SEQ ID NO: 557
  source 1..479: note = XENP39246 Chain 2 - 1A7[CD28]_H1.14_L1.71_scFv(GKPG S)4_Fc(216)_IgG1_C220S/PVA_/S267K/S364K/E357Q

SEQ ID NO: 715 - protein, 216 aa, synthetic construct; identical to SEQ ID NO: 589
  source 1..216: note = XENP39246 Chain 3 - P72_G02[PSMA]_L0 Light Chain

SEQ ID NO: 716 - protein, 451 aa, synthetic construct; identical to SEQ ID NO: 590
  source 1..451: note = XENP39247 Chain 1 - P75_F01[PSMA]_H0_IgG1_pI(-)_Iso steric_A_/PVA_/S267K/L368D/K370S

SEQ ID NO: 717 - protein, 479 aa, synthetic construct; identical to SEQ ID NO: 609
  source 1..479: note = XENP39247 Chain 2 - 1A7[CD28]_H1L1_scFv(GKPGS)4_Fc( 216)_IgG1_C220S/PVA_/S267K/S364K/E357Q

SEQ ID NO: 718 - protein, 215 aa, synthetic construct; identical to SEQ ID NO: 592
  source 1..215: note = XENP39247 Chain 3 - P75_F01[PSMA]_L0 Light Chain

SEQ ID NO: 719 - protein, 451 aa, synthetic construct; identical to SEQ ID NO: 590
  source 1..451: note = XENP39248 Chain 1 - P75_F01[PSMA]_H0_IgG1_pI(-)_Iso steric_A_/PVA_/S267K/L368D/K370S

SEQ ID NO: 720 - protein, 479 aa, synthetic construct; identical to SEQ ID NO: 554
  source 1..479: note = XENP39248 Chain 2 - 1A7[CD28]_H1.14_L1_scFv(GKPGS)4 _Fc(216)_IgG1_C220S/PVA_/S267K/S364K/E357Q

SEQ ID NO: 721 - protein, 215 aa, synthetic construct; identical to SEQ ID NO: 592
  source 1..215: note = XENP39248 Chain 3 - P75_F01[PSMA]_L0 Light Chain

SEQ ID NO: 722 - protein, 451 aa, synthetic construct; identical to SEQ ID NO: 590
  source 1..451: note = XENP39249 Chain 1 - P75_F01[PSMA]_H0_IgG1_pI(-)_Iso steric_A_/PVA_/S267K/L368D/K370S

SEQ ID NO: 723 - protein, 479 aa, synthetic construct; identical to SEQ ID NO: 615
  source 1..479: note = XENP39249 Chain 2 - 1A7[CD28]_H1.1_L1.71_scFv(GKPGS )4_Fc(216)_IgG1_C220S/PVA_/S267K/S364K/E357Q

SEQ ID NO: 724 - protein, 215 aa, synthetic construct; identical to SEQ ID NO: 592
  source 1..215: note = XENP39249 Chain 3 - P75_F01[PSMA]_L0 Light Chain

SEQ ID NO: 725 - protein, 451 aa, synthetic construct; identical to SEQ ID NO: 590
  source 1..451: note = XENP39250 Chain 1 - P75_F01[PSMA]_H0_IgG1_pI(-)_Iso steric_A_/PVA_/S267K/L368D/K370S

SEQ ID NO: 726 - protein, 479 aa, synthetic construct; identical to SEQ ID NO: 557
  source 1..479: note = XENP39250 Chain 2 - 1A7[CD28]_H1.14_L1.71_scFv(GKPG S)4_Fc(216)_IgG1_C220S/PVA_/S267K/S364K/E357Q

SEQ ID NO: 727 - protein, 215 aa, synthetic construct; identical to SEQ ID NO: 592
  source 1..215: note = XENP39250 Chain 3 - P75_F01[PSMA]_L0 Light Chain

SEQ ID NO: 728 - protein, 451 aa, synthetic construct; identical to SEQ ID NO: 593
  source 1..451: note = XENP39251 Chain 1 - P72_F07V2[PSMA]_H0_IgG1_pI(-)_I sosteric_A_/PVA_/S267K/L368D/K370S

SEQ ID NO: 729 - protein, 479 aa, synthetic construct; identical to SEQ ID NO: 609
  source 1..479: note = XENP39251 Chain 2 - 1A7[CD28]_H1L1_scFv(GKPGS)4_Fc( 216)_IgG1_C220S/PVA_/S267K/S364K/E357Q

SEQ ID NO: 730 - protein, 214 aa, synthetic construct; identical to SEQ ID NO: 586
  source 1..214: note = XENP39251 Chain 3 - P72_F07V2[PSMA]_L0 Light Chain

SEQ ID NO: 731 - protein, 451 aa, synthetic construct; identical to SEQ ID NO: 593
  source 1..451: note = XENP39252 Chain 1 - P72_F07V2[PSMA]_H0_IgG1_pI(-)_I sosteric_A_/PVA_/S267K/L368D/K370S

SEQ ID NO: 732 - protein, 479 aa, synthetic construct; identical to SEQ ID NO: 554
  source 1..479: note = XENP39252 Chain 2 - 1A7[CD28]_H1.14_L1_scFv(GKPGS)4 _Fc(216)_IgG1_C220S/PVA_/S267K/S364K/E357Q

SEQ ID NO: 733 - protein, 214 aa, synthetic construct; identical to SEQ ID NO: 586
  source 1..214: note = XENP39252 Chain 3 - P72_F07V2[PSMA]_L0 Light Chain

SEQ ID NO: 734 - protein, 451 aa, synthetic construct; identical to SEQ ID NO: 593
  source 1..451: note = XENP39253 Chain 1 - P72_F07V2[PSMA]_H0_IgG1_pI(-)_I sosteric_A_/PVA_/S267K/L368D/K370S

SEQ ID NO: 735 - protein, 479 aa, synthetic construct; identical to SEQ ID NO: 615
  source 1..479: note = XENP39253 Chain 2 - 1A7[CD28]_H1.1_L1.71_scFv(GKPGS )4_Fc(216)_IgG1_C220S/PVA_/S267K/S364K/E357Q

SEQ ID NO: 736 - protein, 214 aa, synthetic construct; identical to SEQ ID NO: 586
  source 1..214: note = XENP39253 Chain 3 - P72_F07V2[PSMA]_L0 Light Chain

SEQ ID NO: 737 - protein, 451 aa, synthetic construct; identical to SEQ ID NO: 593
  source 1..451: note = XENP39254 Chain 1 - P72_F07V2[PSMA]_H0_IgG1_pI(-)_I sosteric_A_/PVA_/S267K/L368D/K370S

SEQ ID NO: 738 - protein, 479 aa, synthetic construct; identical to SEQ ID NO: 557
  source 1..479: note = XENP39254 Chain 2 - 1A7[CD28]_H1.14_L1.71_scFv(GKPG S)4_Fc(216)_IgG1_C220S/PVA_/S267K/S364K/E357Q

SEQ ID NO: 739 - protein, 214 aa, synthetic construct; identical to SEQ ID NO: 586
  source 1..214: note = XENP39254 Chain 3 - P72_F07V2[PSMA]_L0 Light Chain

SEQ ID NO: 740 - protein, 453 aa, synthetic construct; identical to SEQ ID NO: 596
  source 1..453: note = XENP39255 Chain 1 - P72_G02V2[PSMA]_H0_IgG1_pI(-)_I sosteric_A_/PVA_/S267K/L368D/K370S

SEQ ID NO: 741 - protein, 479 aa, synthetic construct; identical to SEQ ID NO: 609
  source 1..479: note = XENP39255 Chain 2 - 1A7[CD28]_H1L1_scFv(GKPGS)4_Fc( 216)_IgG1_C220S/PVA_/S267K/S364K/E357Q

SEQ ID NO: 742 - protein, 216 aa, synthetic construct; identical to SEQ ID NO: 589
  source 1..216: note = XENP39255 Chain 3 - P72_G02V2[PSMA]_L0 Light Chain

SEQ ID NO: 743 - protein, 453 aa, synthetic construct; identical to SEQ ID NO: 596
  source 1..453: note = XENP39256 Chain 1 - P72_G02V2[PSMA]_H0_IgG1_pI(-)_I sosteric_A_/PVA_/S267K/L368D/K370S

SEQ ID NO: 744 - protein, 479 aa, synthetic construct; identical to SEQ ID NO: 554
  source 1..479: note = XENP39256 Chain 2 - 1A7[CD28]_H1.14_L1_scFv(GKPGS)4 _Fc(216)_IgG1_C220S/PVA_/S267K/S364K/E357Q

SEQ ID NO: 745 - protein, 216 aa, synthetic construct; identical to SEQ ID NO: 589
  source 1..216: note = XENP39256 Chain 3 - P72_G02V2[PSMA]_L0 Light Chain

SEQ ID NO: 746 - protein, 453 aa, synthetic construct; identical to SEQ ID NO: 596
  source 1..453: note = XENP39257 Chain 1 - P72_G02V2[PSMA]_H0_IgG1_pI(-)_I sosteric_A_/PVA_/S267K/L368D/K370S

SEQ ID NO: 747 - protein, 479 aa, synthetic construct; identical to SEQ ID NO: 615
  source 1..479: note = XENP39257 Chain 2 - 1A7[CD28]_H1.1_L1.71_scFv(GKPGS )4_Fc(216)_IgG1_C220S/PVA_/S267K/S364K/E357Q

SEQ ID NO: 748 - protein, 216 aa, synthetic construct; identical to SEQ ID NO: 589
  source 1..216: note = XENP39257 Chain 3 - P72_G02V2[PSMA]_L0 Light Chain

SEQ ID NO: 749 - protein, 453 aa, synthetic construct; identical to SEQ ID NO: 596
  source 1..453: note = XENP39258 Chain 1 - P72_G02V2[PSMA]_H0_IgG1_pI(-)_I sosteric_A_/PVA_/S267K/L368D/K370S

SEQ ID NO: 750 - protein, 479 aa, synthetic construct; identical to SEQ ID NO: 557
  source 1..479: note = XENP39258 Chain 2 - 1A7[CD28]_H1.14_L1.71_scFv(GKPG S)4_Fc(216)_IgG1_C220S/PVA_/S267K/S364K/E357Q

SEQ ID NO: 751 - protein, 216 aa, synthetic construct; identical to SEQ ID NO: 589
  source 1..216: note = XENP39258 Chain 3 - P72_G02V2[PSMA]_L0 Light Chain

SEQ ID NO: 752 - protein, 451 aa, synthetic construct; identical to SEQ ID NO: 599
  source 1..451: note = XENP39259 Chain 1 - P75_F01V2[PSMA]_H0_IgG1_pI(-)_I sosteric_A_/PVA_/S267K/L368D/K370S

SEQ ID NO: 753 - protein, 479 aa, synthetic construct; identical to SEQ ID NO: 609
  source 1..479: note = XENP39259 Chain 2 - 1A7[CD28]_H1L1_scFv(GKPGS)4_Fc( 216)_IgG1_C220S/PVA_/S267K/S364K/E357Q

SEQ ID NO: 754 - protein, 215 aa, synthetic construct; identical to SEQ ID NO: 592
  source 1..215: note = XENP39259 Chain 3 - P75_F01V2[PSMA]_L0 Light Chain

SEQ ID NO: 755 - protein, 451 aa, synthetic construct; identical to SEQ ID NO: 599
  source 1..451: note = XENP39260 Chain 1 - P75_F01V2[PSMA]_H0_IgG1_pI(-)_I sosteric_A_/PVA_/S267K/L368D/K370S

SEQ ID NO: 756 - protein, 479 aa, synthetic construct; identical to SEQ ID NO: 554
  source 1..479: note = XENP39260 Chain 2 - 1A7[CD28]_H1.14_L1_scFv(GKPGS)4 _Fc(216)_IgG1_C220S/PVA_/S267K/S364K/E357Q

SEQ ID NO: 757 - protein, 215 aa, synthetic construct; identical to SEQ ID NO: 592
  source 1..215: note = XENP39260 Chain 3 - P75_F01V2[PSMA]_L0 Light Chain

SEQ ID NO: 758 - protein, 451 aa, synthetic construct; identical to SEQ ID NO: 599
  source 1..451: note = XENP39261 Chain 1 - P75_F01V2[PSMA]_H0_IgG1_pI(-)_I sosteric_A_/PVA_/S267K/L368D/K370S

SEQ ID NO: 759 - protein, 479 aa, synthetic construct; identical to SEQ ID NO: 615
  source 1..479: note = XENP39261 Chain 2 - 1A7[CD28]_H1.1_L1.71_scFv(GKPGS )4_Fc(216)_IgG1_C220S/PVA_/S267K/S364K/E357Q

SEQ ID NO: 760 - protein, 215 aa, synthetic construct; identical to SEQ ID NO: 592
  source 1..215: note = XENP39261 Chain 3 - P75_F01V2[PSMA]_L0 Light Chain

SEQ ID NO: 761 - protein, 451 aa, synthetic construct; identical to SEQ ID NO: 599
  source 1..451: note = XENP39262 Chain 1 - P75_F01V2[PSMA]_H0_IgG1_pI(-)_I sosteric_A_/PVA_/S267K/L368D/K370S

SEQ ID NO: 762 - protein, 479 aa, synthetic construct; identical to SEQ ID NO: 557
  source 1..479: note = XENP39262 Chain 2 - 1A7[CD28]_H1.14_L1.71_scFv(GKPG S)4_Fc(216)_IgG1_C220S/PVA_/S267K/S364K/E357Q

SEQ ID NO: 763 - protein, 215 aa, synthetic construct; identical to SEQ ID NO: 592
  source 1..215: note = XENP39262 Chain 3 - P75_F01V2[PSMA]_L0 Light Chain

SEQ ID NO: 764 - protein, 450 aa, synthetic construct; identical to SEQ ID NO: 602
  source 1..450: note = XENP39263 Chain 1 - PSMB896-HC-G100A[PSMA]_H0_IgG1_ pI(-)_Isosteric_A_/PVA_/S267K/L368D/K370S

SEQ ID NO: 765 - protein, 479 aa, synthetic construct; identical to SEQ ID NO: 609
  source 1..479: note = XENP39263 Chain 2 - 1A7[CD28]_H1L1_scFv(GKPGS)4_Fc( 216)_IgG1_C220S/PVA_/S267K/S364K/E357Q

SEQ ID NO: 766 - protein, 216 aa, synthetic construct; identical to SEQ ID NO: 552
  source 1..216: note = XENP39263 Chain 3 - PSMB896-HC-G100A[PSMA]_L0 Light Chain

SEQ ID NO: 767 - protein, 450 aa, synthetic construct; identical to SEQ ID NO: 602
  source 1..450: note = XENP39264 Chain 1 - PSMB896-HC-G100A[PSMA]_H0_IgG1_ pI(-)_Isosteric_A_/PVA_/S267K/L368D/K370S

SEQ ID NO: 768 - protein, 479 aa, synthetic construct; identical to SEQ ID NO: 554
  source 1..479: note = XENP39264 Chain 2 - 1A7[CD28]_H1.14_L1_scFv(GKPGS)4 _Fc(216)_IgG1_C220S/PVA_/S267K/S364K/E357Q

SEQ ID NO: 769 - protein, 216 aa, synthetic construct; identical to SEQ ID NO: 552
  source 1..216: note = XENP39264 Chain 3 - PSMB896-HC-G100A[PSMA]_L0 Light Chain

SEQ ID NO: 770 - protein, 450 aa, synthetic construct; identical to SEQ ID NO: 602
  source 1..450: note = XENP39265 Chain 1 - PSMB896-HC-G100A[PSMA]_H0_IgG1_ pI(-)_Isosteric_A_/PVA_/S267K/L368D/K370S

SEQ ID NO: 771 - protein, 479 aa, synthetic construct; identical to SEQ ID NO: 615
  source 1..479: note = XENP39265 Chain 2 - 1A7[CD28]_H1.1_L1.71_scFv(GKPGS )4_Fc(216)_IgG1_C220S/PVA_/S267K/S364K/E357Q

SEQ ID NO: 772 - protein, 216 aa, synthetic construct; identical to SEQ ID NO: 552
  source 1..216: note = XENP39265 Chain 3 - PSMB896-HC-G100A[PSMA]_L0 Light Chain

SEQ ID NO: 773 - protein, 450 aa, synthetic construct; identical to SEQ ID NO: 602
  source 1..450: note = XENP39266 Chain 1 - PSMB896-HC-G100A[PSMA]_H0_IgG1_ pI(-)_Isosteric_A_/PVA_/S267K/L368D/K370S

SEQ ID NO: 774 - protein, 479 aa, synthetic construct; identical to SEQ ID NO: 557
  source 1..479: note = XENP39266 Chain 2 - 1A7[CD28]_H1.14_L1.71_scFv(GKPG S)4_Fc(216)_IgG1_C220S/PVA_/S267K/S364K/E357Q

SEQ ID NO: 775 - protein, 216 aa, synthetic construct; identical to SEQ ID NO: 552
  source 1..216: note = XENP39266 Chain 3 - PSMB896-HC-G100A[PSMA]_L0 Light Chain

SEQ ID NO: 776 - protein, 449 aa, synthetic construct; identical to SEQ ID NO: 605
  source 1..449: note = XENP39267 Chain 1 - 011A11[PSMA]_H0_IgG1_pI(-)_Isos teric_A_/PVA_/S267K/L368D/K370S

SEQ ID NO: 777 - protein, 479 aa, synthetic construct; identical to SEQ ID NO: 609
  source 1..479: note = XENP39267 Chain 2 - 1A7[CD28]_H1L1_scFv(GKPGS)4_Fc( 216)_IgG1_C220S/PVA_/S267K/S364K/E357Q

SEQ ID NO: 778 - protein, 215 aa, synthetic construct; identical to SEQ ID NO: 549
  source 1..215: note = XENP39267 Chain 3 - 011A11[PSMA]_L0 Light Chain

SEQ ID NO: 779 - protein, 449 aa, synthetic construct; identical to SEQ ID NO: 605
  source 1..449: note = XENP39268 Chain 1 - 011A11[PSMA]_H0_IgG1_pI(-)_Isos teric_A_/PVA_/S267K/L368D/K370S

SEQ ID NO: 780 - protein, 479 aa, synthetic construct; identical to SEQ ID NO: 554
  source 1..479: note = XENP39268 Chain 2 - 1A7[CD28]_H1.14_L1_scFv(GKPGS)4 _Fc(216)_IgG1_C220S/PVA_/S267K/S364K/E357Q

SEQ ID NO: 781 - protein, 215 aa, synthetic construct; identical to SEQ ID NO: 549
  source 1..215: note = XENP39268 Chain 3 - 011A11[PSMA]_L0 Light Chain

SEQ ID NO: 782 - protein, 449 aa, synthetic construct; identical to SEQ ID NO: 605
  source 1..449: note = XENP39269 Chain 1 - 011A11[PSMA]_H0_IgG1_pI(-)_Isos teric_A_/PVA_/S267K/L368D/K370S

SEQ ID NO: 783 - protein, 479 aa, synthetic construct; identical to SEQ ID NO: 615
  source 1..479: note = XENP39269 Chain 2 - 1A7[CD28]_H1.1_L1.71_scFv(GKPGS )4_Fc(216)_IgG1_C220S/PVA_/S267K/S364K/E357Q

SEQ ID NO: 784 - protein, 215 aa, synthetic construct; identical to SEQ ID NO: 549
  source 1..215: note = XENP39269 Chain 3 - 011A11[PSMA]_L0 Light Chain

SEQ ID NO: 785 - protein, 449 aa, synthetic construct; identical to SEQ ID NO: 605
  source 1..449: note = XENP39270 Chain 1 - 011A11[PSMA]_H0_IgG1_pI(-)_Isos teric_A_/PVA_/S267K/L368D/K370S

SEQ ID NO: 786 - protein, 479 aa, synthetic construct; identical to SEQ ID NO: 557
  source 1..479: note = XENP39270 Chain 2 - 1A7[CD28]_H1.14_L1.71_scFv(GKPG S)4_Fc(216)_IgG1_C220S/PVA_/S267K/S364K/E357Q

SEQ ID NO: 787 - protein, 215 aa, synthetic construct; identical to SEQ ID NO: 549
  source 1..215: note = XENP39270 Chain 3 - 011A11[PSMA]_L0 Light Chain

SEQ ID NO: 788 - protein, 449 aa, synthetic construct; identical to SEQ ID NO: 578
  source 1..449: note = XENP39274 Chain 1 - P72_A10V2[PSMA]_H0_IgG1_pI(-)_I sosteric_A_/PVA_/S267K/L368D/K370S

SEQ ID NO: 789 - protein, 479 aa, synthetic construct
  source 1..479: note = XENP39274 Chain 2 - 1A7[CD28]_L1_H1.14_scFv(GKPGS)4 _Fc(216)_IgG1_C220S/PVA_/S267K/S364K/E357Q
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKGKPGSGKPGSGKPGSGKPGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 790 - protein, 213 aa, synthetic construct; identical to SEQ ID NO: 344
  source 1..213: note = XENP39274 Chain 3 - P72_A10V2[PSMA]_L0 Light Chain

SEQ ID NO: 791 - protein, 455 aa, synthetic construct; identical to SEQ ID NO: 581
  source 1..455: note = XENP39275 Chain 1 - P72_D01V2[PSMA]_H0_IgG1_pI(-)_I sosteric_A_/PVA_/S267K/L368D/K370S

SEQ ID NO: 792 - protein, 479 aa, synthetic construct; identical to SEQ ID NO: 789
  source 1..479: note = XENP39275 Chain 2 - 1A7[CD28]_L1_H1.14_scFv(GKPGS)4 _Fc(216)_IgG1_C220S/PVA_/S267K/S364K/E357Q

SEQ ID NO: 793 - protein, 216 aa, synthetic construct; identical to SEQ ID NO: 568
  source 1..216: note = XENP39275 Chain 3 - P72_D01V2[PSMA]_L0 Light Chain

SEQ ID NO: 794 - protein, 451 aa, synthetic construct; identical to SEQ ID NO: 584
  source 1..451: note = XENP39276 Chain 1 - P72_F07[PSMA]_H0_IgG1_pI(-)_Iso steric_A_/PVA_/S267K/L368D/K370S

SEQ ID NO: 795 - protein, 479 aa, synthetic construct; identical to SEQ ID NO: 789
  source 1..479: note = XENP39276 Chain 2 - 1A7[CD28]_L1_H1.14_scFv(GKPGS)4 _Fc(216)_IgG1_C220S/PVA_/S267K/S364K/E357Q

SEQ ID NO: 796 - protein, 214 aa, synthetic construct; identical to SEQ ID NO: 586
  source 1..214: note = XENP39276 Chain 3 - P72_F07[PSMA]_L0 Light Chain

SEQ ID NO: 797 - protein, 449 aa, synthetic construct; identical to SEQ ID NO: 578
  source 1..449: note = XENP39277 Chain 1 - P72_A10V2[PSMA]_H0_IgG1_pI(-)_I sosteric_A_/PVA_/S267K/L368D/K370S

SEQ ID NO: 798 - protein, 479 aa, synthetic construct
  source 1..479: note = XENP39277 Chain 2 - 1A7[CD28]_L1.71_H1.14_scFv(GKPG S)4_Fc(216)_IgG1_C220S/PVA_/S267K/S364K/E357Q
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGKPGSGKPGSGKPGSGKPGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 799 - protein, 213 aa, synthetic construct; identical to SEQ ID NO: 344
  source 1..213: note = XENP39277 Chain 3 - P72_A10V2[PSMA]_L0 Light Chain

SEQ ID NO: 800 - protein, 455 aa, synthetic construct; identical to SEQ ID NO: 581
  source 1..455: note = XENP39278 Chain 1 - P72_D01V2[PSMA]_H0_IgG1_pI(-)_I sosteric_A_/PVA_/S267K/L368D/K370S

SEQ ID NO: 801 - protein, 479 aa, synthetic construct; identical to SEQ ID NO: 798
  source 1..479: note = XENP39278 Chain 2 - 1A7[CD28]_L1.71_H1.14_scFv(GKPG S)4_Fc(216)_IgG1_C220S/PVA_/S267K/S364K/E357Q

SEQ ID NO: 802 - protein, 216 aa, synthetic construct; identical to SEQ ID NO: 568
  source 1..216: note = XENP39278 Chain 3 - P72_D01V2[PSMA]_L0 Light Chain

SEQ ID NO: 803 - protein, 451 aa, synthetic construct; identical to SEQ ID NO: 584
  source 1..451: note = XENP39279 Chain 1 - P72_F07[PSMA]_H0_IgG1_pI(-)_Iso steric_A_/PVA_/S267K/L368D/K370S

SEQ ID NO: 804 - protein, 479 aa, synthetic construct; identical to SEQ ID NO: 798
  source 1..479: note = XENP39279 Chain 2 - 1A7[CD28]_L1.71_H1.14_scFv(GKPG S)4_Fc(216)_IgG1_C220S/PVA_/S267K/S364K/E357Q

SEQ ID NO: 805 - protein, 214 aa, synthetic construct; identical to SEQ ID NO: 586
  source 1..214: note = XENP39279 Chain 3 - P72_F07[PSMA]_L0 Light Chain

SEQ ID NO: 806 - protein, 449 aa, synthetic construct; identical to SEQ ID NO: 578
  source 1..449: note = XENP40470 Chain 1 - P72_A10V2[PSMA]_H0_IgG1_pI(-)_I sosteric_A_/PVA_/S267K/L368D/K370S

SEQ ID NO: 807 - protein, 479 aa, synthetic construct
  source 1..479: note = XENP40470 Chain 2 - 1A7[CD28]_H1.1_L1_scFv(GKPGS)4_ Fc(216)_IgG1_C220S/PVA_/S267K/S364K/E357Q
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 808 - protein, 213 aa, synthetic construct; identical to SEQ ID NO: 344
  source 1..213: note = XENP40470 Chain 3 - P72_A10V2[PSMA]_L0 Light Chain

SEQ ID NO: 809 - protein, 455 aa, synthetic construct; identical to SEQ ID NO: 566
  source 1..455: note = XENP41406 Chain 1 - P72_D01[PSMA]_H0_IgG1_pI(-)_Iso steric_A_/PVA_/S267K/L368D/K370S

SEQ ID NO: 810 - protein, 479 aa, synthetic construct
  source 1..479: note = XENP41406 Chain 2 - 1A7[CD28]_L1.71_H1_scFv(GKPGS)4 _Fc(216)_IgG1_C220S/PVA_/S267K/S364K/E357Q
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGKPGSGKPGSGKPGSGKPGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 811 - protein, 216 aa, synthetic construct; identical to SEQ ID NO: 568
  source 1..216: note = XENP41406 Chain 3 - P72_D01[PSMA]_L0 Light Chain

SEQ ID NO: 812 - protein, 450 aa, synthetic construct; identical to SEQ ID NO: 602
  source 1..450: note = XENP42268 Chain 1 - C28PB247 (PSMB896-HC-G100A[PSMA ]_H0_IgG1_pI(-)_Isosteric_A_/PVA_/S267K/L368D/K370S

SEQ ID NO: 813 - protein, 479 aa, synthetic construct; identical to SEQ ID NO: 564
  source 1..479: note = XENP42268 Chain 2 - 1A7[CD28]_H1_L1.71_scFv(GKPGS)4 _Fc(216)_IgG1_C220S/PVA_/S267K/S364K/E357Q)

SEQ ID NO: 814 - protein, 216 aa, synthetic construct; identical to SEQ ID NO: 552
  source 1..216: note = XENP42268 Chain 3 - PSMB896-HC-G100A[PSMA]_L0 Light Chain

SEQ ID NO: 815 - protein, 478 aa, synthetic construct
  source 1..478: note = C28PB405 Chain 1 - 1A7[CD28]_h1.14_l1-spFv
EVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 816 - protein, 450 aa, synthetic construct; identical to SEQ ID NO: 343
  source 1..450: note = C28PB405 Chain 2 - HC1 (AAS_knob3): P72_A10V2

SEQ ID NO: 817 - protein, 213 aa, synthetic construct; identical to SEQ ID NO: 344
  source 1..213: note = C28PB405 Chain 3 - LC: P72_A10V2

SEQ ID NO: 818 - protein, 478 aa, synthetic construct; identical to SEQ ID NO: 815
  source 1..478: note = C28PB404 Chain 1 - 1A7[CD28]_h1.14_l1-spFv

SEQ ID NO: 819 - protein, 451 aa, synthetic construct; identical to SEQ ID NO: 551
  source 1..451: note = C28PB404 Chain 2 - HC1 (AAS_knob3): PSMB896-G100A

SEQ ID NO: 820 - protein, 216 aa, synthetic construct; identical to SEQ ID NO: 552
  source 1..216: note = C28PB404 Chain 3 - LC: PSMB896-G100A

SEQ ID NO: 821 - protein, 478 aa, synthetic construct
  source 1..478: note = C28PB403 Chain 1 - 1A7_H1.1_L1-spFv
EVQLLESGGGLVQPGGSLRLSCAASGFTFSSYYMSWVRQAPGCGLEWVSTISGSGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 822 - protein, 450 aa, synthetic construct; identical to SEQ ID NO: 343
  source 1..450: note = C28PB403 Chain 2 - HC1 (AAS_knob3): P72_A10V2

SEQ ID NO: 823 - protein, 213 aa, synthetic construct; identical to SEQ ID NO: 344
  source 1..213: note = C28PB403 Chain 3 - LC: P72_A10V2

SEQ ID NO: 824 - protein, 478 aa, synthetic construct; identical to SEQ ID NO: 821
  source 1..478: note = C28PB402 Chain 1 - 1A7_H1.1_L1-spFv

SEQ ID NO: 825 - protein, 451 aa, synthetic construct; identical to SEQ ID NO: 551
  source 1..451: note = C28PB402 Chain 2 - HC1 (AAS_knob3): PSMB896-G100A

SEQ ID NO: 826 - protein, 216 aa, synthetic construct; identical to SEQ ID NO: 552
  source 1..216: note = C28PB402 Chain 3 - LC: PSMB896-G100A

SEQ ID NO: 827 - protein, 478 aa, synthetic construct
  source 1..478: note = C28PB401 Chain 1 - 1A7[CD28]_h1.14_l1-spFv
EVQLLESGGGLVQPGGSLRLSCAASGFTFSSYYMSWVRQAPGCGLEWVSTISESGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 828 - protein, 450 aa, synthetic construct; identical to SEQ ID NO: 343
  source 1..450: note = C28PB401 Chain 2 - HC1 (AAS_knob3): P72_A10V2

SEQ ID NO: 829 - protein, 213 aa, synthetic construct; identical to SEQ ID NO: 344
  source 1..213: note = C28PB401 Chain 3 - LC: P72_A10V2

SEQ ID NO: 830 - protein, 478 aa, synthetic construct; identical to SEQ ID NO: 342
  source 1..478: note = C28PB400 Chain 1 - 1A7[CD28]_H1_L1.71-spFv

SEQ ID NO: 831 - protein, 451 aa, synthetic construct; identical to SEQ ID NO: 551
  source 1..451: note = C28PB400 Chain 2 - HC1 (AAS_knob3): PSMB896-G100A

SEQ ID NO: 832 - protein, 216 aa, synthetic construct; identical to SEQ ID NO: 552
  source 1..216: note = C28PB400 Chain 3 - LC: PSMB896-G100A

SEQ ID NO: 833 - protein, 480 aa, synthetic construct
  source 1..480: note = C28PB343 Chain 1 - 1A7[CD28]_H1L1-HL-scFv
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 834 - protein, 456 aa, synthetic construct
  source 1..456: note = C28PB343 Chain 2 - HC1 (knob): PSMA_P72_D01V2
EVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCAREGVGADYGDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 835 - protein, 216 aa, synthetic construct; identical to SEQ ID NO: 568
  source 1..216: note = C28PB343 Chain 3 - LC: PSMA_P72_D01V2

SEQ ID NO: 836 - protein, 480 aa, synthetic construct; identical to SEQ ID NO: 833
  source 1..480: note = C28PB342 Chain 1 - 1A7[CD28]_H1L1-HL-scFv

SEQ ID NO: 837 - protein, 450 aa, synthetic construct; identical to SEQ ID NO: 343
  source 1..450: note = C28PB342 Chain 2 - HC1 (knob): PSMA_P72_A10V2

SEQ ID NO: 838 - protein, 213 aa, synthetic construct; identical to SEQ ID NO: 344
  source 1..213: note = C28PB342 Chain 3 - LC: PSMA_P72_A10V2

SEQ ID NO: 839 - protein, 480 aa, synthetic construct; identical to SEQ ID NO: 833
  source 1..480: note = C28PB341 Chain 1 - 1A7[CD28]_H1L1-HL-scFv

SEQ ID NO: 840 - protein, 451 aa, synthetic construct; identical to SEQ ID NO: 551
  source 1..451: note = C28PB341 Chain 2 - HC1 (knob): PSMB896-HC-G100A

SEQ ID NO: 841 - protein, 216 aa, synthetic construct; identical to SEQ ID NO: 552
  source 1..216: note = C28PB341 Chain 3 - LC: PSMB896-HC-G100A

SEQ ID NO: 842 - protein, 480 aa, synthetic construct; identical to SEQ ID NO: 351
  source 1..480: note = C28PB331 Chain 1 - 1A7[CD28]_H1_L1.71-HL-scFv

SEQ ID NO: 843 - protein, 456 aa, synthetic construct; identical to SEQ ID NO: 834
  source 1..456: note = C28PB331 Chain 2 - HC1 (knob): PSMA_P72_D01V2

SEQ ID NO: 844 - protein, 216 aa, synthetic construct; identical to SEQ ID NO: 568
  source 1..216: note = C28PB331 Chain 3 - LC: PSMA_P72_D01V2

SEQ ID NO: 845 - protein, 480 aa, synthetic construct; identical to SEQ ID NO: 351
  source 1..480: note = C28PB329 Chain 1 - 1A7[CD28]_H1_L1.71-HL-scFv

SEQ ID NO: 846 - protein, 451 aa, synthetic construct; identical to SEQ ID NO: 551
  source 1..451: note = C28PB329 Chain 2 - HC1 (knob): PSMB896-HC-G100A

SEQ ID NO: 847 - protein, 216 aa, synthetic construct; identical to SEQ ID NO: 552
  source 1..216: note = C28PB329 Chain 3 - LC: PSMB896-HC-G100A

SEQ ID NO: 848 - protein, 480 aa, synthetic construct
  source 1..480: note = C28PB319 Chain 1 - 1A7[CD28]_H1.1_L1.71-HL-scFv
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 849 - protein, 456 aa, synthetic construct; identical to SEQ ID NO: 834
  source 1..456: note = C28PB319 Chain 2 - HC1 (knob): PSMA_P72_D01V2

SEQ ID NO: 850 - protein, 216 aa, synthetic construct; identical to SEQ ID NO: 568
  source 1..216: note = C28PB319 Chain 3 - LC: PSMA_P72_D01V2

SEQ ID NO: 851 - protein, 480 aa, synthetic construct; identical to SEQ ID NO: 848
  source 1..480: note = C28PB318 Chain 1 - 1A7[CD28]_H1.1_L1.71-HL-scFv

SEQ ID NO: 852 - protein, 450 aa, synthetic construct; identical to SEQ ID NO: 343
  source 1..450: note = C28PB318 Chain 2 - HC1 (knob): PSMA_P72_A10V2

SEQ ID NO: 853 - protein, 213 aa, synthetic construct; identical to SEQ ID NO: 344
  source 1..213: note = C28PB318 Chain 3 - LC: PSMA_P72_A10V2

SEQ ID NO: 854 - protein, 480 aa, synthetic construct
  source 1..480: note = C28PB307 Chain 1 - 1A7[CD28]_H1.14_L1-HL-scFv
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 855 - protein, 456 aa, synthetic construct; identical to SEQ ID NO: 834
  source 1..456: note = C28PB307 Chain 2 - HC1 (knob): PSMA_P72_D01V2

SEQ ID NO: 856 - protein, 216 aa, synthetic construct; identical to SEQ ID NO: 568
  source 1..216: note = C28PB307 Chain 3 - LC: PSMA_P72_D01V2

SEQ ID NO: 857 - protein, 480 aa, synthetic construct; identical to SEQ ID NO: 854
  source 1..480: note = C28PB306 Chain 1 - 1A7[CD28]_H1.14_L1-HL-scFv

SEQ ID NO: 858 - protein, 450 aa, synthetic construct; identical to SEQ ID NO: 343
  source 1..450: note = C28PB306 Chain 2 - HC1 (knob): PSMA_P72_A10V2

SEQ ID NO: 859 - protein, 213 aa, synthetic construct; identical to SEQ ID NO: 344
  source 1..213: note = C28PB306 Chain 3 - LC: PSMA_P72_A10V2

SEQ ID NO: 860 - protein, 480 aa, synthetic construct
  source 1..480: note = C28PB295 Chain 1 - 1A7[CD28]_H1.14_L1.71-HL-scFv
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 861 - protein, 456 aa, synthetic construct; identical to SEQ ID NO: 834
  source 1..456: note = C28PB295 Chain 2 - HC1 (knob): PSMA_P72_D01V2

SEQ ID NO: 862 - protein, 216 aa, synthetic construct; identical to SEQ ID NO: 568
  source 1..216: note = C28PB295 Chain 3 - LC: PSMA_P72_D01V2

SEQ ID NO: 863 - protein, 480 aa, synthetic construct; identical to SEQ ID NO: 860
  source 1..480: note = C28PB294 Chain 1 - 1A7[CD28]_H1.14_L1.71-HL-scFv

SEQ ID NO: 864 - protein, 450 aa, synthetic construct; identical to SEQ ID NO: 343
  source 1..450: note = C28PB294 Chain 2 - HC1 (knob): PSMA_P72_A10V2

SEQ ID NO: 865 - protein, 213 aa, synthetic construct; identical to SEQ ID NO: 344
  source 1..213: note = C28PB294 Chain 3 - LC: PSMA_P72_A10V2

SEQ ID NO: 866 - protein, 451 aa, synthetic construct; identical to SEQ ID NO: 551
  source 1..451: note = C28PB329 Chain 1 - PSMB896-HC-G100A[PSMA]_H0_IgG1_L 234A/L235A/D265S/T366S/L368A/Y407V/H435R/Y436F

SEQ ID NO: 867 - protein, 480 aa, synthetic construct; identical to SEQ ID NO: 351
  source 1..480: note = C28PB329 Chain 2 - 1A7[CD28]_H1_L1.71_scFv(GKPGS)4_ Fc(216)_IgG1_C220S/L234A/L235A/D265S/T366W

SEQ ID NO: 868 - protein, 216 aa, synthetic construct; identical to SEQ ID NO: 552
  source 1..216: note = C28PB329 Chain 3 - PSMB896-HC-G100A[PSMA]_L0 Light Chain

SEQ ID NO: 869 - protein, 121 aa, synthetic construct; identical to SEQ ID NO: 19
  source 1..121: note = 1A7[CD28]_H1.1

SEQ ID NO: 870 - protein, 121 aa, synthetic construct
  source 1..121: note = 1A7[CD28]_H1.2
EVQLLESGGGLVQPGGSLRLSCAASGFTFSSYAMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 871 - protein, 121 aa, synthetic construct
  source 1..121: note = 1A7[CD28]_H1.3
EVQLLESGGGLVQPGGSLRLSCAASGFTFSSYAMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 872 - protein, 121 aa, synthetic construct
  source 1..121: note = 1A7[CD28]_H1.4
EVQLLESGGGLVQPGGSLRLSCAASGFTFSSYAMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 873 - protein, 121 aa, synthetic construct
  source 1..121: note = 1A7[CD28]_H1.5
EVQLLESGGGLVQPGGSLRLSCAASGFTFSSYAMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 874 - protein, 121 aa, synthetic construct
  source 1..121: note = 1A7[CD28]_H1.6
EVQLLESGGGLVQPGGSLRLSCAASGFTFSSYAMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 875 - protein, 121 aa, synthetic construct
  source 1..121: note = 1A7[CD28]_H1.7
EVQLLESGGGLVQPGGSLRLSCAASGFTFSSYAMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 876 - protein, 121 aa, synthetic construct
  source 1..121: note = 1A7[CD28]_H1.8
EVQLLESGGGLVQPGGSLRLSCAASGFTFSSYAMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 877 - protein, 121 aa, synthetic construct
  source 1..121: note = 1A7[CD28]_H1.9
EVQLLESGGGLVQPGGSLRLSCAASGFTFSSYAMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 878 - protein, 121 aa, synthetic construct
  source 1..121: note = 1A7[CD28]_H1.10
EVQLLESGGGLVQPGGSLRLSCAASGFTFSSYYMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 879 - protein, 121 aa, synthetic construct
  source 1..121: note = 1A7[CD28]_H1.11
EVQLLESGGGLVQPGGSLRLSCAASGFTFSSYYMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 880 - protein, 121 aa, synthetic construct
  source 1..121: note = 1A7[CD28]_H1.12
EVQLLESGGGLVQPGGSLRLSCAASGFTFSSYYMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 881 - protein, 121 aa, synthetic construct
  source 1..121: note = 1A7[CD28]_H1.13
EVQLLESGGGLVQPGGSLRLSCAASGFTFSSYYMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 882 - protein, 121 aa, synthetic construct; identical to SEQ ID NO: 23
  source 1..121: note = 1A7[CD28]_H1.14

SEQ ID NO: 883 - protein, 121 aa, synthetic construct
  source 1..121: note = 1A7[CD28]_H1.15
EVQLLESGGGLVQPGGSLRLSCAASGFTFSSYYMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 884 - protein, 121 aa, synthetic construct
  source 1..121: note = 1A7[CD28]_H1.16
EVQLLESGGGLVQPGGSLRLSCAASGFTFSSYYMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 885 - protein, 121 aa, synthetic construct
  source 1..121: note = 1A7[CD28]_H1.17
EVQLLESGGGLVQPGGSLRLSCAASGFTFSSYYMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 886 - protein, 121 aa, synthetic construct
  source 1..121: note = 1A7[CD28]_H1.18
EVQLLESGGGLVQPGGSLRLSCAASGFTFSSYAMSWVRQAPGKGLEWVSTIDDSGDSTYYADSVKGRFTISRDNSKNTLYLQMNSLRAEDTAVYYCAKSGPGLRQVGFDYWGQGTLVTVSS

SEQ ID NO: 887 - protein, 121 aa, synthetic construct
  source 1..121: note = 1A7[CD28]_H1.19
EVQLLESGGGLVQPGGSLRLSCAASGFTFSSYAMSWVRQAPGKGLEWVSTIDESGDSTYYADSVKGRFTISRDNSKNTLYLQMNSLRAEDTAVYYCAKSGPGLRQVGFDYWGQGTLVTVSS

SEQ ID NO: 888 - protein, 121 aa, synthetic construct
  source 1..121: note = 1A7[CD28]_H1.20
EVQLLESGGGLVQPGGSLRLSCAASGFTFSSYAMSWVRQAPGKGLEWVSTIDGSGTSTYYADSVKGRFTISRDNSKNTLYLQMNSLRAEDTAVYYCAKSGPGLRQVGFDYWGQGTLVTVSS

SEQ ID NO: 889 - protein, 121 aa, synthetic construct
  source 1..121: note = 1A7[CD28]_H1.21
EVQLLESGGGLVQPGGSLRLSCAASGFTFSSYAMSWVRQAPGKGLEWVSTIDGSGYSTYYADSVKGRFTISRDNSKNTLYLQMNSLRAEDTAVYYCAKSGPGLRQVGFDYWGQGTLVTVSS

SEQ ID NO: 890 - protein, 121 aa, synthetic construct
  source 1..121: note = 1A7[CD28]_H1.22
EVQLLESGGGLVQPGGSLRLSCAASGFTFSSYAMSWVRQAPGKGLEWVSTIDGSGDYTYYADSVKGRFTISRDNSKNTLYLQMNSLRAEDTAVYYCAKSGPGLRQVGFDYWGQGTLVTVSS

SEQ ID NO: 891 - protein, 121 aa, synthetic construct
  source 1..121: note = 1A7[CD28]_H1.23
EVQLLESGGGLVQPGGSLRLSCAASGFTFSSYAMSWVRQAPGKGLEWVSTIEDSGDSTYYADSVKGRFTISRDNSKNTLYLQMNSLRAEDTAVYYCAKSGPGLRQVGFDYWGQGTLVTVSS

SEQ ID NO: 892 - protein, 121 aa, synthetic construct
  source 1..121: note = 1A7[CD28]_H1.24
EVQLLESGGGLVQPGGSLRLSCAASGFTFSSYAMSWVRQAPGKGLEWVSTIEESGDSTYYADSVKGRFTISRDNSKNTLYLQMNSLRAEDTAVYYCAKSGPGLRQVGFDYWGQGTLVTVSS

SEQ ID NO: 893 - protein, 121 aa, synthetic construct
  source 1..121: note = 1A7[CD28]_H1.25
EVQLLESGGGLVQPGGSLRLSCAASGFTFSSYAMSWVRQAPGKGLEWVSTIEGSGTSTYYADSVKGRFTISRDNSKNTLYLQMNSLRAEDTAVYYCAKSGPGLRQVGFDYWGQGTLVTVSS

SEQ ID NO: 894 - protein, 121 aa, synthetic construct
  source 1..121: note = 1A7[CD28]_H1.26
EVQLLESGGGLVQPGGSLRLSCAASGFTFSSYAMSWVRQAPGKGLEWVSTIEGSGYSTYYADSVKGRFTISRDNSKNTLYLQMNSLRAEDTAVYYCAKSGPGLRQVGFDYWGQGTLVTVSS

SEQ ID NO: 895 - protein, 121 aa, synthetic construct
  source 1..121: note = 1A7[CD28]_H1.27
EVQLLESGGGLVQPGGSLRLSCAASGFTFSSYAMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 896 - protein, 121 aa, synthetic construct
  source 1..121: note = 1A7[CD28]_H1.28
EVQLLESGGGLVQPGGSLRLSCAASGFTFSSYAMSWVRQAPGKGLEWVSTIYDSGDSTYYADSVKGRFTISRDNSKNTLYLQMNSLRAEDTAVYYCAKSGPGLRQVGFDYWGQGTLVTVSS

SEQ ID NO: 897 - protein, 121 aa, synthetic construct
  source 1..121: note = 1A7[CD28]_H1.29
EVQLLESGGGLVQPGGSLRLSCAASGFTFSSYAMSWVRQAPGKGLEWVSTIYESGDSTYYADSVKGRFTISRDNSKNTLYLQMNSLRAEDTAVYYCAKSGPGLRQVGFDYWGQGTLVTVSS

SEQ ID NO: 898 - protein, 121 aa, synthetic construct
  source 1..121: note = 1A7[CD28]_H1.30
EVQLLESGGGLVQPGGSLRLSCAASGFTFSSYAMSWVRQAPGKGLEWVSTIYGSGTSTYYADSVKGRFTISRDNSKNTLYLQMNSLRAEDTAVYYCAKSGPGLRQVGFDYWGQGTLVTVSS

SEQ ID NO: 899 - protein, 121 aa, synthetic construct
  source 1..121: note = 1A7[CD28]_H1.31
EVQLLESGGGLVQPGGSLRLSCAASGFTFSSYAMSWVRQAPGKGLEWVSTIYGSGYSTYYADSVKGRFTISRDNSKNTLYLQMNSLRAEDTAVYYCAKSGPGLRQVGFDYWGQGTLVTVSS

SEQ ID NO: 900 - protein, 121 aa, synthetic construct
  source 1..121: note = 1A7[CD28]_H1.32
EVQLLESGGGLVQPGGSLRLSCAASGFTFSSYAMSWVRQAPGKGLEWVSTIYGSGDYTYYADSVKGRFTISRDNSKNTLYLQMNSLRAEDTAVYYCAKSGPGLRQVGFDYWGQGTLVTVSS

SEQ ID NO: 901 - protein, 121 aa, synthetic construct
  source 1..121: note = 1A7[CD28]_H1.33
EVQLLESGGGLVQPGGSLRLSCAASGFTFSSYAMSWVRQAPGKGLEWVSTISDSGTSTYYADSVKGRFTISRDNSKNTLYLQMNSLRAEDTAVYYCAKSGPGLRQVGFDYWGQGTLVTVSS

SEQ ID NO: 902 - protein, 121 aa, synthetic construct
  source 1..121: note = 1A7[CD28]_H1.34
EVQLLESGGGLVQPGGSLRLSCAASGFTFSSYAMSWVRQAPGKGLEWVSTISDSGYSTYYADSVKGRFTISRDNSKNTLYLQMNSLRAEDTAVYYCAKSGPGLRQVGFDYWGQGTLVTVSS

SEQ ID NO: 903 - protein, 121 aa, synthetic construct
  source 1..121: note = 1A7[CD28]_H1.35
EVQLLESGGGLVQPGGSLRLSCAASGFTFSSYAMSWVRQAPGKGLEWVSTISDSGDYTYYADSVKGRFTISRDNSKNTLYLQMNSLRAEDTAVYYCAKSGPGLRQVGFDYWGQGTLVTVSS

SEQ ID NO: 904 - protein, 121 aa, synthetic construct
  source 1..121: note = 1A7[CD28]_H1.36
EVQLLESGGGLVQPGGSLRLSCAASGFTFSSYAMSWVRQAPGKGLEWVSTISESGTSTYYADSVKGRFTISRDNSKNTLYLQMNSLRAEDTAVYYCAKSGPGLRQVGFDYWGQGTLVTVSS

SEQ ID NO: 905 - protein, 121 aa, synthetic construct
  source 1..121: note = 1A7[CD28]_H1.37
EVQLLESGGGLVQPGGSLRLSCAASGFTFSSYAMSWVRQAPGKGLEWVSTISESGYSTYYADSVKGRFTISRDNSKNTLYLQMNSLRAEDTAVYYCAKSGPGLRQVGFDYWGQGTLVTVSS

SEQ ID NO: 906 - protein, 121 aa, synthetic construct
  source 1..121: note = 1A7[CD28]_H1.38
EVQLLESGGGLVQPGGSLRLSCAASGFTFSSYAMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 907 - protein, 121 aa, synthetic construct
  source 1..121: note = 1A7[CD28]_H1.39
EVQLLESGGGLVQPGGSLRLSCAASGFTFSSYAMSWVRQAPGKGLEWVSTISGSGTYTYYADSVKGRFTISRDNSKNTLYLQMNSLRAEDTAVYYCAKSGPGLRQVGFDYWGQGTLVTVSS

SEQ ID NO: 908 - protein, 121 aa, synthetic construct
  source 1..121: note = 1A7[CD28]_H1.40
EVQLLESGGGLVQPGGSLRLSCAASGFTFSSYAMSWVRQAPGKGLEWVSTISGSGYYTYYADSVKGRFTISRDNSKNTLYLQMNSLRAEDTAVYYCAKSGPGLRQVGFDYWGQGTLVTVSS

SEQ ID NO: 909 - protein, 121 aa, synthetic construct
  source 1..121: note = 1A7[CD28]_H1.41
EVQLLESGGGLVQPGGSLRLSCAASGFTFSSYYMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 910 - protein, 121 aa, synthetic construct
  source 1..121: note = 1A7[CD28]_H1.42
EVQLLESGGGLVQPGGSLRLSCAASGFTFSSYYMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 911 - protein, 121 aa, synthetic construct
  source 1..121: note = 1A7[CD28]_H1.43
EVQLLESGGGLVQPGGSLRLSCAASGFSFSGNYMTWVRQAPGKGLEWVATITADSDATYYADSVKGRFTISRDNSKNTLYLQMNSLRAEDTAVYYCAKSGPGLRQVGFDYWGQGTLVTVSS

SEQ ID NO: 912 - protein, 121 aa, synthetic construct
  source 1..121: note = 1A7[CD28]_H1.44
EVQLLESGGGLVQPGGSLRLSCAASGFTFSGYSMNWVRQAPGKGLEWVATIYANGSYTYYADSVKGRFTISRDNSKNTLYLQMNSLRAEDTAVYYCAKSGPGLRQVGFDYWGQGTLVTVSS

SEQ ID NO: 913 - protein, 121 aa, synthetic construct
  source 1..121: note = 1A7[CD28]_H1.45
EVQLLESGGGLVQPGGSLRLSCAASGFNFEEYSMNWVRQAPGKGLEWVATITYNGDYTYYADSVKGRFTISRDNSKNTLYLQMNSLRAEDTAVYYCAKSGPGLRQVGFDYWGQGTLVTVSS

SEQ ID NO: 914 - protein, 121 aa, synthetic construct
  source 1..121: note = 1A7[CD28]_H1.46
EVQLLESGGGLVQPGGSLRLSCAASGFSFRTYYMTWVRQAPGKGLEWVATITSDGDYTYYADSVKGRFTISRDNSKNTLYLQMNSLRAEDTAVYYCAKSGPGLRQVGFDYWGQGTLVTVSS

SEQ ID NO: 915 - protein, 121 aa, synthetic construct
  source 1..121: note = 1A7[CD28]_H1.47
EVQLLESGGGLVQPGGSLRLSCAASGFSFKGYSMNWVRQAPGKGLEWVATIYASSDSTYYADSVKGRFTISRDNSKNTLYLQMNSLRAEDTAVYYCAKSGPGLRQVGFDYWGQGTLVTVSS

SEQ ID NO: 916 - protein, 121 aa, synthetic construct
  source 1..121: note = 1A7[CD28]_H1.48
EVQLLESGGGLVQPGGSLRLSCAASGFSFGEYSMNWVRQAPGKGLEWVSTIYADGDYTYYADSVKGRFTISRDNSKNTLYLQMNSLRAEDTAVYYCAKSGPGLRQVGFDYWGQGTLVTVSS

SEQ ID NO: 917 - protein, 121 aa, synthetic construct
  source 1..121: note = 1A7[CD28]_H1.49
EVQLLESGGGLVQPGGSLRLSCAASGFTFTDYSMNWVRQAPGKGLEWVATIYADSSSTYYADSVKGRFTISRDNSKNTLYLQMNSLRAEDTAVYYCAKSGPGLRQVGFDYWGQGTLVTVSS

SEQ ID NO: 918 - protein, 121 aa, synthetic construct
  source 1..121: note = 1A7[CD28]_H1.50
EVQLLESGGGLVQPGGSLRLSCAASGFTFGGYSMNWVRQAPGKGLEWVATIYYDSTTTYYADSVKGRFTISRDNSKNTLYLQMNSLRAEDTAVYYCAKSGPGLRQVGFDYWGQGTLVTVSS

SEQ ID NO: 919 - protein, 121 aa, synthetic construct
  source 1..121: note = 1A7[CD28]_H1.51
EVQLLESGGGLVQPGGSLRLSCAASGFTFGAYSMNWVRQAPGKGLEWVSTIYNDGATTYYADSVKGRFTISRDNSKNTLYLQMNSLRAEDTAVYYCAKSGPGLRQVGFDYWGQGTLVTVSS

SEQ ID NO: 920 - protein, 121 aa, synthetic construct
  source 1..121: note = 1A7[CD28]_H1.52
EVQLLESGGGLVQPGGSLRLSCAASGFSFEAYSMNWVRQAPGKGLEWVATIYYDSSYTYYADSVKGRFTISRDNSKNTLYLQMNSLRAEDTAVYYCAKSGPGLRQVGFDYWGQGTLVTVSS

SEQ ID NO: 921 - protein, 121 aa, synthetic construct
  source 1..121: note = 1A7[CD28]_H1.53
EVQLLESGGGLVQPGGSLRLSCAASGFSFGSYYMSWVRQAPGKGLEWVASIYYGGYDTYYADSVKGRFTISRDNSKNTLYLQMNSLRAEDTAVYYCAKSGPGLRQVGFDYWGQGTLVTVSS

SEQ ID NO: 922 - protein, 121 aa, synthetic construct
  source 1..121: note = 1A7[CD28]_H1.54
EVQLLESGGGLVQPGGSLRLSCAASGFNFAEYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 923 - protein, 121 aa, synthetic construct
  source 1..121: note = 1A7[CD28]_H1.55
EVQLLESGGGLVQPGGSLRLSCAASGFTFGAYSINWVRQAPGKGLEWVATIYYDGSYTYYADSVKGRFTISRDNSKNTLYLQMNSLRAEDTAVYYCAKSGPGLRQVGFDYWGQGTLVTVSS

SEQ ID NO: 924 - protein, 121 aa, synthetic construct
  source 1..121: note = 1A7[CD28]_H1.56
EVQLLESGGGLVQPGGSLRLSCAASGFTFSRYYINWVRQAPGKGLEWVASIYDGGADTYYADSVKGRFTISRDNSKNTLYLQMNSLRAEDTAVYYCAKSGPGLRQVGFDYWGQGTLVTVSS

SEQ ID NO: 925 - protein, 121 aa, synthetic construct
  source 1..121: note = 1A7[CD28]_H1.57
EVQLLESGGGLVQPGGSLRLSCAASGFSFGTYSINWVRQAPGKGLEWVSTIYYDGASTYYADSVKGRFTISRDNSKNTLYLQMNSLRAEDTAVYYCAKSGPGLRQVGFDYWGQGTLVTVSS

SEQ ID NO: 926 - protein, 121 aa, synthetic construct
  source 1..121: note = 1A7[CD28]_H1.58
EVQLLESGGGLVQPGGSLRLSCAASGFTFGKYSISWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 927 - protein, 121 aa, synthetic construct
  source 1..121: note = 1A7[CD28]_H1.59
EVQLLESGGGLVQPGGSLRLSCAASGFSFEKYYISWVRQAPGKGLEWVASIYDGSYDTYYADSVKGRFTISRDNSKNTLYLQMNSLRAEDTAVYYCAKSGPGLRQVGFDYWGQGTLVTVSS

SEQ ID NO: 928 - protein, 121 aa, synthetic construct
  source 1..121: note = 1A7[CD28]_H1.60
EVQLLESGGGLVQPGGSLRLSCAASGFSFGTYSMNWVRQAPGKGLEWVATIDYDGSNTYYADSVKGRFTISRDNSKNTLYLQMNSLRAEDTAVYYCAKSGPGLRQVGFDYWGQGTLVTVSS

SEQ ID NO: 929 - protein, 121 aa, synthetic construct
  source 1..121: note = 1A7[CD28]_H1.61
EVQLLESGGGLVQPGGSLRLSCAASGFSFAAYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 930 - protein, 121 aa, synthetic construct
  source 1..121: note = 1A7[CD28]_H1.62
EVQLLESGGGLVQPGGSLRLSCAASGFTFTTYSMTWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 931 - protein, 121 aa, synthetic construct
  source 1..121: note = 1A7[CD28]_H1.63
EVQLLESGGGLVQPGGSLRLSCAASGFSFKDYSMNWVRQAPGKGLEWVATIYYDGTYTYYADSVKGRFTISRDNSKNTLYLQMNSLRAEDTAVYYCAKSGPGLRQVGFDYWGQGTLVTVSS

SEQ ID NO: 932 - protein, 121 aa, synthetic construct
  source 1..121: note = 1A7[CD28]_H1.64
EVQLLESGGGLVQPGGSLRLSCAASGFSFAAYSMNWVRQAPGKGLEWVATIYAGGAYTYYADSVKGRFTISRDNSKNTLYLQMNSLRAEDTAVYYCAKSGPGLRQVGFDYWGQGTLVTVSS

SEQ ID NO: 933 - protein, 121 aa, synthetic construct
  source 1..121: note = 1A7[CD28]_H1.65
EVQLLESGGGLVQPGGSLRLSCAASGFNFAEYYISWVRQAPGKGLEWVASIYNGGSDTYYADSVKGRFTISRDNSKNTLYLQMNSLRAEDTAVYYCAKSGPGLRQVGFDYWGQGTLVTVSS

SEQ ID NO: 934 - protein, 121 aa, synthetic construct
  source 1..121: note = 1A7[CD28]_H1.66
EVQLLESGGGLVQPGGSLRLSCAASGFSLGEYYMNWVRQAPGKGLEWVSTIDYDGTYTYYADSVKGRFTISRDNSKNTLYLQMNSLRAEDTAVYYCAKSGPGLRQVGFDYWGQGTLVTVSS

SEQ ID NO: 935 - protein, 121 aa, synthetic construct
  source 1..121: note = 1A7[CD28]_H1.67
EVQLLESGGGLVQPGGSLRLSCAASGFTFNEYYITWVRQAPGKGLEWVSSIYSSSYDTYYADSVKGRFTISRDNSKNTLYLQMNSLRAEDTAVYYCAKSGPGLRQVGFDYWGQGTLVTVSS

SEQ ID NO: 936 - protein, 121 aa, synthetic construct
  source 1..121: note = 1A7[CD28]_H1.68
EVQLLESGGGLVQPGGSLRLSCAASGFTFKDYYITWVRQAPGKGLEWVSSIYTSGYDTYYADSVKGRFTISRDNSKNTLYLQMNSLRAEDTAVYYCAKSGPGLRQVGFDYWGQGTLVTVSS

SEQ ID NO: 937 - protein, 121 aa, synthetic construct
  source 1..121: note = 1A7[CD28]_H1.69
EVQLLESGGGLVQPGGSLRLSCAASGFSFGKYSINWVRQAPGKGLEWVATIYSDGTDTYYADSVKGRFTISRDNSKNTLYLQMNSLRAEDTAVYYCAKSGPGLRQVGFDYWGQGTLVTVSS

SEQ ID NO: 938 - protein, 107 aa, synthetic construct
  source 1..107: note = 1A7[CD28]_L1.1
DIQMTQSPSSLSASVGDRVTITCRASQSISAYLNWYQQKPGKAPKLLIYAASSLQSGVPSRFSGSGSGTDFTLTISSLQPEDFATYYCQQSYSTPFTFGQGTKLEIK

SEQ ID NO: 939 - protein, 107 aa, synthetic construct
  source 1..107: note = 1A7[CD28]_L1.2
DIQMTQSPSSLSASVGDRVTITCRASQSISDYLNWYQQKPGKAPKLLIYAASSLQSGVPSRFSGSGSGTDFTLTISSLQPEDFATYYCQQSYSTPFTFGQGTKLEIK

SEQ ID NO: 940 - protein, 107 aa, synthetic construct
  source 1..107: note = 1A7[CD28]_L1.3
DIQMTQSPSSLSASVGDRVTITCRASQSISGYLNWYQQKPGKAPKLLIYAASSLQSGVPSRFSGSGSGTDFTLTISSLQPEDFATYYCQQSYSTPFTFGQGTKLEIK

SEQ ID NO: 941 - protein, 107 aa, synthetic construct
  source 1..107: note = 1A7[CD28]_L1.4
DIQMTQSPSSLSASVGDRVTITCRASQSISHYLNWYQQKPGKAPKLLIYAASSLQSGVPSRFSGSGSGTDFTLTISSLQPEDFATYYCQQSYSTPFTFGQGTKLEIK

SEQ ID NO: 942 - protein, 107 aa, synthetic construct
  source 1..107: note = 1A7[CD28]_L1.5
DIQMTQSPSSLSASVGDRVTITCRASQSISKYLNWYQQKPGKAPKLLIYAASSLQSGVPSRFSGSGSGTDFTLTISSLQPEDFATYYCQQSYSTPFTFGQGTKLEIK

SEQ ID NO: 943 - protein, 107 aa, synthetic construct
  source 1..107: note = 1A7[CD28]_L1.6
DIQMTQSPSSLSASVGDRVTITCRASQSISNYLNWYQQKPGKAPKLLIYAASSLQSGVPSRFSGSGSGTDFTLTISSLQPEDFATYYCQQSYSTPFTFGQGTKLEIK

SEQ ID NO: 944 - protein, 107 aa, synthetic construct
  source 1..107: note = 1A7[CD28]_L1.7
DIQMTQSPSSLSASVGDRVTITCRASQSISQYLNWYQQKPGKAPKLLIYAASSLQSGVPSRFSGSGSGTDFTLTISSLQPEDFATYYCQQSYSTPFTFGQGTKLEIK

SEQ ID NO: 945 - protein, 107 aa, synthetic construct
  source 1..107: note = 1A7[CD28]_L1.8
DIQMTQSPSSLSASVGDRVTITCRASQSISTYLNWYQQKPGKAPKLLIYAASSLQSGVPSRFSGSGSGTDFTLTISSLQPEDFATYYCQQSYSTPFTFGQGTKLEIK

SEQ ID NO: 946 - protein, 107 aa, synthetic construct
  source 1..107: note = 1A7[CD28]_L1.9
DIQMTQSPSSLSASVGDRVTITCRASQSISVYLNWYQQKPGKAPKLLIYAASSLQSGVPSRFSGSGSGTDFTLTISSLQPEDFATYYCQQSYSTPFTFGQGTKLEIK

SEQ ID NO: 947 - protein, 107 aa, synthetic construct
  source 1..107: note = 1A7[CD28]_L1.10
DIQMTQSPSSLSASVGDRVTITCRASQSISYYLNWYQQKPGKAPKLLIYAASSLQSGVPSRFSGSGSGTDFTLTISSLQPEDFATYYCQQSYSTPFTFGQGTKLEIK

SEQ ID NO: 948 - protein, 107 aa, synthetic construct
  source 1..107: note = 1A7[CD28]_L1.11
DIQMTQSPSSLSASVGDRVTITCRASQSISSALNWYQQKPGKAPKLLIYAASSLQSGVPSRFSGSGSGTDFTLTISSLQPEDFATYYCQQSYSTPFTFGQGTKLEIK

SEQ ID NO: 949 - protein, 107 aa, synthetic construct
  source 1..107: note = 1A7[CD28]_L1.12
DIQMTQSPSSLSASVGDRVTITCRASQSISSDLNWYQQKPGKAPKLLIYAASSLQSGVPSRFSGSGSGTDFTLTISSLQPEDFATYYCQQSYSTPFTFGQGTKLEIK

SEQ ID NO: 950 - protein, 107 aa, synthetic construct
  source 1..107: note = 1A7[CD28]_L1.13
DIQMTQSPSSLSASVGDRVTITCRASQSISSFLNWYQQKPGKAPKLLIYAASSLQSGVPSRFSGSGSGTDFTLTISSLQPEDFATYYCQQSYSTPFTFGQGTKLEIK

SEQ ID NO: 951 - protein, 107 aa, synthetic construct
  source 1..107: note = 1A7[CD28]_L1.14
DIQMTQSPSSLSASVGDRVTITCRASQSISSHLNWYQQKPGKAPKLLIYAASSLQSGVPSRFSGSGSGTDFTLTISSLQPEDFATYYCQQSYSTPFTFGQGTKLEIK

SEQ ID NO: 952 - protein, 107 aa, synthetic construct
  source 1..107: note = 1A7[CD28]_L1.15
DIQMTQSPSSLSASVGDRVTITCRASQSISSKLNWYQQKPGKAPKLLIYAASSLQSGVPSRFSGSGSGTDFTLTISSLQPEDFATYYCQQSYSTPFTFGQGTKLEIK

SEQ ID NO: 953 - protein, 107 aa, synthetic construct
  source 1..107: note = 1A7[CD28]_L1.16
DIQMTQSPSSLSASVGDRVTITCRASQSISSLLNWYQQKPGKAPKLLIYAASSLQSGVPSRFSGSGSGTDFTLTISSLQPEDFATYYCQQSYSTPFTFGQGTKLEIK

SEQ ID NO: 954 - protein, 107 aa, synthetic construct
  source 1..107: note = 1A7[CD28]_L1.17
DIQMTQSPSSLSASVGDRVTITCRASQSISSNLNWYQQKPGKAPKLLIYAASSLQSGVPSRFSGSGSGTDFTLTISSLQPEDFATYYCQQSYSTPFTFGQGTKLEIK

SEQ ID NO: 955 - protein, 107 aa, synthetic construct
  source 1..107: note = 1A7[CD28]_L1.18
DIQMTQSPSSLSASVGDRVTITCRASQSISSQLNWYQQKPGKAPKLLIYAASSLQSGVPSRFSGSGSGTDFTLTISSLQPEDFATYYCQQSYSTPFTFGQGTKLEIK

SEQ ID NO: 956 - protein, 107 aa, synthetic construct
  source 1..107: note = 1A7[CD28]_L1.19
DIQMTQSPSSLSASVGDRVTITCRASQSISSSLNWYQQKPGKAPKLLIYAASSLQSGVPSRFSGSGSGTDFTLTISSLQPEDFATYYCQQSYSTPFTFGQGTKLEIK

SEQ ID NO: 957 - protein, 107 aa, synthetic construct
  source 1..107: note = 1A7[CD28]_L1.20
DIQMTQSPSSLSASVGDRVTITCRASQSISSWLNWYQQKPGKAPKLLIYAASSLQSGVPSRFSGSGSGTDFTLTISSLQPEDFATYYCQQSYSTPFTFGQGTKLEIK

SEQ ID NO: 958 - protein, 107 aa, synthetic construct
  source 1..107: note = 1A7[CD28]_L1.21
DIQMTQSPSSLSASVGDRVTITCRASQSISSYLAWYQQKPGKAPKLLIYAASSLQSGVPSRFSGSGSGTDFTLTISSLQPEDFATYYCQQSYSTPFTFGQGTKLEIK

SEQ ID NO: 959 - protein, 107 aa, synthetic construct
  source 1..107: note = 1A7[CD28]_L1.22
DIQMTQSPSSLSASVGDRVTITCRASQSISSYLDWYQQKPGKAPKLLIYAASSLQSGVPSRFSGSGSGTDFTLTISSLQPEDFATYYCQQSYSTPFTFGQGTKLEIK

SEQ ID NO: 960 - protein, 107 aa, synthetic construct
  source 1..107: note = 1A7[CD28]_L1.23
DIQMTQSPSSLSASVGDRVTITCRASQSISSYLGWYQQKPGKAPKLLIYAASSLQSGVPSRFSGSGSGTDFTLTISSLQPEDFATYYCQQSYSTPFTFGQGTKLEIK

SEQ ID NO: 961 - protein, 107 aa, synthetic construct
  source 1..107: note = 1A7[CD28]_L1.24
DIQMTQSPSSLSASVGDRVTITCRASQSISSYLHWYQQKPGKAPKLLIYAASSLQSGVPSRFSGSGSGTDFTLTISSLQPEDFATYYCQQSYSTPFTFGQGTKLEIK

SEQ ID NO: 962 - protein, 107 aa, synthetic construct
  source 1..107: note = 1A7[CD28]_L1.25
DIQMTQSPSSLSASVGDRVTITCRASQSISSYLQWYQQKPGKAPKLLIYAASSLQSGVPSRFSGSGSGTDFTLTISSLQPEDFATYYCQQSYSTPFTFGQGTKLEIK

SEQ ID NO: 963 - protein, 107 aa, synthetic construct
  source 1..107: note = 1A7[CD28]_L1.26
DIQMTQSPSSLSASVGDRVTITCRASQSISSYLSWYQQKPGKAPKLLIYAASSLQSGVPSRFSGSGSGTDFTLTISSLQPEDFATYYCQQSYSTPFTFGQGTKLEIK

SEQ ID NO: 964 - protein, 107 aa, synthetic construct
  source 1..107: note = 1A7[CD28]_L1.27
DIQMTQSPSSLSASVGDRVTITCRASQSISSYLTWYQQKPGKAPKLLIYAASSLQSGVPSRFSGSGSGTDFTLTISSLQPEDFATYYCQQSYSTPFTFGQGTKLEIK

SEQ ID NO: 965 - protein, 107 aa, synthetic construct
  source 1..107: note = 1A7[CD28]_L1.28
DIQMTQSPSSLSASVGDRVTITCRASQSISSYLYWYQQKPGKAPKLLIYAASSLQSGVPSRFSGSGSGTDFTLTISSLQPEDFATYYCQQSYSTPFTFGQGTKLEIK

SEQ ID NO: 966 - protein, 107 aa, synthetic construct
  source 1..107: note = 1A7[CD28]_L1.29
DIQMTQSPSSLSASVGDRVTITCRASQSISSYLNWYQQKPGKAPKLLIYDASSLQSGVPSRFSGSGSGTDFTLTISSLQPEDFATYYCQQSYSTPFTFGQGTKLEIK

SEQ ID NO: 967 - protein, 107 aa, synthetic construct
  source 1..107: note = 1A7[CD28]_L1.30
DIQMTQSPSSLSASVGDRVTITCRASQSISSYLNWYQQKPGKAPKLLIYGASSLQSGVPSRFSGSGSGTDFTLTISSLQPEDFATYYCQQSYSTPFTFGQGTKLEIK

SEQ ID NO: 968 - protein, 107 aa, synthetic construct
  source 1..107: note = 1A7[CD28]_L1.31
DIQMTQSPSSLSASVGDRVTITCRASQSISSYLNWYQQKPGKAPKLLIYKASSLQSGVPSRFSGSGSGTDFTLTISSLQPEDFATYYCQQSYSTPFTFGQGTKLEIK

SEQ ID NO: 969 - protein, 107 aa, synthetic construct
  source 1..107: note = 1A7[CD28]_L1.32
DIQMTQSPSSLSASVGDRVTITCRASQSISSYLNWYQQKPGKAPKLLIYLASSLQSGVPSRFSGSGSGTDFTLTISSLQPEDFATYYCQQSYSTPFTFGQGTKLEIK

SEQ ID NO: 970 - protein, 107 aa, synthetic construct
  source 1..107: note = 1A7[CD28]_L1.33
DIQMTQSPSSLSASVGDRVTITCRASQSISSYLNWYQQKPGKAPKLLIYQASSLQSGVPSRFSGSGSGTDFTLTISSLQPEDFATYYCQQSYSTPFTFGQGTKLEIK

SEQ ID NO: 971 - protein, 107 aa, synthetic construct
  source 1..107: note = 1A7[CD28]_L1.34
DIQMTQSPSSLSASVGDRVTITCRASQSISSYLNWYQQKPGKAPKLLIYSASSLQSGVPSRFSGSGSGTDFTLTISSLQPEDFATYYCQQSYSTPFTFGQGTKLEIK

SEQ ID NO: 972 - protein, 107 aa, synthetic construct
  source 1..107: note = 1A7[CD28]_L1.35
DIQMTQSPSSLSASVGDRVTITCRASQSISSYLNWYQQKPGKAPKLLIYTASSLQSGVPSRFSGSGSGTDFTLTISSLQPEDFATYYCQQSYSTPFTFGQGTKLEIK

SEQ ID NO: 973 - protein, 107 aa, synthetic construct
  source 1..107: note = 1A7[CD28]_L1.36
DIQMTQSPSSLSASVGDRVTITCRASQSISSYLNWYQQKPGKAPKLLIYWASSLQSGVPSRFSGSGSGTDFTLTISSLQPEDFATYYCQQSYSTPFTFGQGTKLEIK

SEQ ID NO: 974 - protein, 107 aa, synthetic construct
  source 1..107: note = 1A7[CD28]_L1.37
DIQMTQSPSSLSASVGDRVTITCRASQSISSYLNWYQQKPGKAPKLLIYYASSLQSGVPSRFSGSGSGTDFTLTISSLQPEDFATYYCQQSYSTPFTFGQGTKLEIK

SEQ ID NO: 975 - protein, 107 aa, synthetic construct
  source 1..107: note = 1A7[CD28]_L1.38
DIQMTQSPSSLSASVGDRVTITCRASQSISSYLNWYQQKPGKAPKLLIYAASALQSGVPSRFSGSGSGTDFTLTISSLQPEDFATYYCQQSYSTPFTFGQGTKLEIK

SEQ ID NO: 976 - protein, 107 aa, synthetic construct
  source 1..107: note = 1A7[CD28]_L1.39
DIQMTQSPSSLSASVGDRVTITCRASQSISSYLNWYQQKPGKAPKLLIYAASDLQSGVPSRFSGSGSGTDFTLTISSLQPEDFATYYCQQSYSTPFTFGQGTKLEIK

SEQ ID NO: 977 - protein, 107 aa, synthetic construct
  source 1..107: note = 1A7[CD28]_L1.40
DIQMTQSPSSLSASVGDRVTITCRASQSISSYLNWYQQKPGKAPKLLIYAASKLQSGVPSRFSGSGSGTDFTLTISSLQPEDFATYYCQQSYSTPFTFGQGTKLEIK

SEQ ID NO: 978 - protein, 107 aa, synthetic construct
  source 1..107: note = 1A7[CD28]_L1.41
DIQMTQSPSSLSASVGDRVTITCRASQSISSYLNWYQQKPGKAPKLLIYAASNLQSGVPSRFSGSGSGTDFTLTISSLQPEDFATYYCQQSYSTPFTFGQGTKLEIK

SEQ ID NO: 979 - protein, 107 aa, synthetic construct
  source 1..107: note = 1A7[CD28]_L1.42
DIQMTQSPSSLSASVGDRVTITCRASQSISSYLNWYQQKPGKAPKLLIYAASQLQSGVPSRFSGSGSGTDFTLTISSLQPEDFATYYCQQSYSTPFTFGQGTKLEIK

SEQ ID NO: 980 - protein, 107 aa, synthetic construct
  source 1..107: note = 1A7[CD28]_L1.43
DIQMTQSPSSLSASVGDRVTITCRASQSISSYLNWYQQKPGKAPKLLIYAASTLQSGVPSRFSGSGSGTDFTLTISSLQPEDFATYYCQQSYSTPFTFGQGTKLEIK

SEQ ID NO: 981 - protein, 107 aa, synthetic construct
  source 1..107: note = 1A7[CD28]_L1.44
DIQMTQSPSSLSASVGDRVTITCRASQSISSYLNWYQQKPGKAPKLLIYAASYLQSGVPSRFSGSGSGTDFTLTISSLQPEDFATYYCQQSYSTPFTFGQGTKLEIK

SEQ ID NO: 982 - protein, 107 aa, synthetic construct
  source 1..107: note = 1A7[CD28]_L1.45
DIQMTQSPSSLSASVGDRVTITCRASQSISSYLNWYQQKPGKAPKLLIYAASSLASGVPSRFSGSGSGTDFTLTISSLQPEDFATYYCQQSYSTPFTFGQGTKLEIK

SEQ ID NO: 983 - protein, 107 aa, synthetic construct
  source 1..107: note = 1A7[CD28]_L1.46
DIQMTQSPSSLSASVGDRVTITCRASQSISSYLNWYQQKPGKAPKLLIYAASSLESGVPSRFSGSGSGTDFTLTISSLQPEDFATYYCQQSYSTPFTFGQGTKLEIK

SEQ ID NO: 984 - protein, 107 aa, synthetic construct
  source 1..107: note = 1A7[CD28]_L1.47
DIQMTQSPSSLSASVGDRVTITCRASQSISSYLNWYQQKPGKAPKLLIYAASSLFSGVPSRFSGSGSGTDFTLTISSLQPEDFATYYCQQSYSTPFTFGQGTKLEIK

SEQ ID NO: 985 - protein, 107 aa, synthetic construct
  source 1..107: note = 1A7[CD28]_L1.48
DIQMTQSPSSLSASVGDRVTITCRASQSISSYLNWYQQKPGKAPKLLIYAASSLHSGVPSRFSGSGSGTDFTLTISSLQPEDFATYYCQQSYSTPFTFGQGTKLEIK

SEQ ID NO: 986 - protein, 107 aa, synthetic construct
  source 1..107: note = 1A7[CD28]_L1.49
DIQMTQSPSSLSASVGDRVTITCRASQSISSYLNWYQQKPGKAPKLLIYAASSLISGVPSRFSGSGSGTDFTLTISSLQPEDFATYYCQQSYSTPFTFGQGTKLEIK

SEQ ID NO: 987 - protein, 107 aa, synthetic construct
  source 1..107: note = 1A7[CD28]_L1.50
DIQMTQSPSSLSASVGDRVTITCRASQSISSYLNWYQQKPGKAPKLLIYAASSLKSGVPSRFSGSGSGTDFTLTISSLQPEDFATYYCQQSYSTPFTFGQGTKLEIK

SEQ ID NO: 988 - protein, 107 aa, synthetic construct
  source 1..107: note = 1A7[CD28]_L1.51
DIQMTQSPSSLSASVGDRVTITCRASQSISSYLNWYQQKPGKAPKLLIYAASSLNSGVPSRFSGSGSGTDFTLTISSLQPEDFATYYCQQSYSTPFTFGQGTKLEIK

SEQ ID NO: 989 - protein, 107 aa, synthetic construct
  source 1..107: note = 1A7[CD28]_L1.52
DIQMTQSPSSLSASVGDRVTITCRASQSISSYLNWYQQKPGKAPKLLIYAASSLSSGVPSRFSGSGSGTDFTLTISSLQPEDFATYYCQQSYSTPFTFGQGTKLEIK

SEQ ID NO: 990 - protein, 107 aa, synthetic construct
  source 1..107: note = 1A7[CD28]_L1.53
DIQMTQSPSSLSASVGDRVTITCRASQSISSYLNWYQQKPGKAPKLLIYAASSLVSGVPSRFSGSGSGTDFTLTISSLQPEDFATYYCQQSYSTPFTFGQGTKLEIK

SEQ ID NO: 991 - protein, 107 aa, synthetic construct
  source 1..107: note = 1A7[CD28]_L1.54
DIQMTQSPSSLSASVGDRVTITCRASQSISSYLNWYQQKPGKAPKLLIYAASSLYSGVPSRFSGSGSGTDFTLTISSLQPEDFATYYCQQSYSTPFTFGQGTKLEIK

SEQ ID NO: 992 - protein, 107 aa, synthetic construct
  source 1..107: note = 1A7[CD28]_L1.55
DIQMTQSPSSLSASVGDRVTITCRASQSISSYLNWYQQKPGKAPKLLIYAASSLQAGVPSRFSGSGSGTDFTLTISSLQPEDFATYYCQQSYSTPFTFGQGTKLEIK

SEQ ID NO: 993 - protein, 107 aa, synthetic construct
  source 1..107: note = 1A7[CD28]_L1.56
DIQMTQSPSSLSASVGDRVTITCRASQSISSYLNWYQQKPGKAPKLLIYAASSLQDGVPSRFSGSGSGTDFTLTISSLQPEDFATYYCQQSYSTPFTFGQGTKLEIK

SEQ ID NO: 994 - protein, 107 aa, synthetic construct
  source 1..107: note = 1A7[CD28]_L1.57
DIQMTQSPSSLSASVGDRVTITCRASQSISSYLNWYQQKPGKAPKLLIYAASSLQGGVPSRFSGSGSGTDFTLTISSLQPEDFATYYCQQSYSTPFTFGQGTKLEIK

SEQ ID NO: 995 - protein, 107 aa, synthetic construct
  source 1..107: note = 1A7[CD28]_L1.58
DIQMTQSPSSLSASVGDRVTITCRASQSISSYLNWYQQKPGKAPKLLIYAASSLQHGVPSRFSGSGSGTDFTLTISSLQPEDFATYYCQQSYSTPFTFGQGTKLEIK

SEQ ID NO: 996 - protein, 107 aa, synthetic construct
  source 1..107: note = 1A7[CD28]_L1.59
DIQMTQSPSSLSASVGDRVTITCRASQSISSYLNWYQQKPGKAPKLLIYAASSLQKGVPSRFSGSGSGTDFTLTISSLQPEDFATYYCQQSYSTPFTFGQGTKLEIK

SEQ ID NO: 997 - protein, 107 aa, synthetic construct
  source 1..107: note = 1A7[CD28]_L1.60
DIQMTQSPSSLSASVGDRVTITCRASQSISSYLNWYQQKPGKAPKLLIYAASSLQQGVPSRFSGSGSGTDFTLTISSLQPEDFATYYCQQSYSTPFTFGQGTKLEIK

SEQ ID NO: 998 - protein, 107 aa, synthetic construct
  source 1..107: note = 1A7[CD28]_L1.61
DIQMTQSPSSLSASVGDRVTITCRASQSISSYLNWYQQKPGKAPKLLIYAASSLQTGVPSRFSGSGSGTDFTLTISSLQPEDFATYYCQQSYSTPFTFGQGTKLEIK

SEQ ID NO: 999 - protein, 107 aa, synthetic construct
  source 1..107: note = 1A7[CD28]_L1.62
DIQMTQSPSSLSASVGDRVTITCRASQSISSYLNWYQQKPGKAPKLLIYAASSLQVGVPSRFSGSGSGTDFTLTISSLQPEDFATYYCQQSYSTPFTFGQGTKLEIK

SEQ ID NO: 1000 - protein, 107 aa, synthetic construct
  source 1..107: note = 1A7[CD28]_L1.63
DIQMTQSPSSLSASVGDRVTITCRASQSISSYLNWYQQKPGKAPKLLIYAASSLQYGVPSRFSGSGSGTDFTLTISSLQPEDFATYYCQQSYSTPFTFGQGTKLEIK

SEQ ID NO: 1001 - protein, 107 aa, synthetic construct
  source 1..107: note = 1A7[CD28]_L1.64
DIQMTQSPSSLSASVGDRVTITCRASQSISSYLNWYQQKPGKAPKLLIYAASSLQSGVPSRFSGSGSGTDFTLTISSLQPEDFATYYCQQAYSTPFTFGQGTKLEIK

SEQ ID NO: 1002 - protein, 107 aa, synthetic construct
  source 1..107: note = 1A7[CD28]_L1.65
DIQMTQSPSSLSASVGDRVTITCRASQSISSYLNWYQQKPGKAPKLLIYAASSLQSGVPSRFSGSGSGTDFTLTISSLQPEDFATYYCQQDYSTPFTFGQGTKLEIK

SEQ ID NO: 1003 - protein, 107 aa, synthetic construct
  source 1..107: note = 1A7[CD28]_L1.66
DIQMTQSPSSLSASVGDRVTITCRASQSISSYLNWYQQKPGKAPKLLIYAASSLQSGVPSRFSGSGSGTDFTLTISSLQPEDFATYYCQQFYSTPFTFGQGTKLEIK

SEQ ID NO: 1004 - protein, 107 aa, synthetic construct
  source 1..107: note = 1A7[CD28]_L1.67
DIQMTQSPSSLSASVGDRVTITCRASQSISSYLNWYQQKPGKAPKLLIYAASSLQSGVPSRFSGSGSGTDFTLTISSLQPEDFATYYCQQHYSTPFTFGQGTKLEIK

SEQ ID NO: 1005 - protein, 107 aa, synthetic construct
  source 1..107: note = 1A7[CD28]_L1.68
DIQMTQSPSSLSASVGDRVTITCRASQSISSYLNWYQQKPGKAPKLLIYAASSLQSGVPSRFSGSGSGTDFTLTISSLQPEDFATYYCQQKYSTPFTFGQGTKLEIK

SEQ ID NO: 1006 - protein, 107 aa, synthetic construct
  source 1..107: note = 1A7[CD28]_L1.69
DIQMTQSPSSLSASVGDRVTITCRASQSISSYLNWYQQKPGKAPKLLIYAASSLQSGVPSRFSGSGSGTDFTLTISSLQPEDFATYYCQQLYSTPFTFGQGTKLEIK

SEQ ID NO: 1007 - protein, 107 aa, synthetic construct
  source 1..107: note = 1A7[CD28]_L1.70
DIQMTQSPSSLSASVGDRVTITCRASQSISSYLNWYQQKPGKAPKLLIYAASSLQSGVPSRFSGSGSGTDFTLTISSLQPEDFATYYCQQTYSTPFTFGQGTKLEIK

SEQ ID NO: 1008 - protein, 107 aa, synthetic construct; identical to SEQ ID NO: 27
  source 1..107: note = 1A7[CD28]_L1.71

SEQ ID NO: 1009 - protein, 107 aa, synthetic construct
  source 1..107: note = 1A7[CD28]_L1.72
DIQMTQSPSSLSASVGDRVTITCRASQSISSYLNWYQQKPGKAPKLLIYAASSLQSGVPSRFSGSGSGTDFTLTISSLQPEDFATYYCQQYYSTPFTFGQGTKLEIK

SEQ ID NO: 1010 - protein, 107 aa, synthetic construct
  source 1..107: note = 1A7[CD28]_L1.73
DIQMTQSPSSLSASVGDRVTITCRASQSISSYLNWYQQKPGKAPKLLIYAASSLQSGVPSRFSGSGSGTDFTLTISSLQPEDFATYYCQQSASTPFTFGQGTKLEIK

SEQ ID NO: 1011 - protein, 107 aa, synthetic construct
  source 1..107: note = 1A7[CD28]_L1.74
DIQMTQSPSSLSASVGDRVTITCRASQSISSYLNWYQQKPGKAPKLLIYAASSLQSGVPSRFSGSGSGTDFTLTISSLQPEDFATYYCQQSDSTPFTFGQGTKLEIK

SEQ ID NO: 1012 - protein, 107 aa, synthetic construct
  source 1..107: note = 1A7[CD28]_L1.75
DIQMTQSPSSLSASVGDRVTITCRASQSISSYLNWYQQKPGKAPKLLIYAASSLQSGVPSRFSGSGSGTDFTLTISSLQPEDFATYYCQQSFSTPFTFGQGTKLEIK

SEQ ID NO: 1013 - protein, 107 aa, synthetic construct
  source 1..107: note = 1A7[CD28]_L1.76
DIQMTQSPSSLSASVGDRVTITCRASQSISSYLNWYQQKPGKAPKLLIYAASSLQSGVPSRFSGSGSGTDFTLTISSLQPEDFATYYCQQSHSTPFTFGQGTKLEIK

SEQ ID NO: 1014 - protein, 107 aa, synthetic construct
  source 1..107: note = 1A7[CD28]_L1.77
DIQMTQSPSSLSASVGDRVTITCRASQSISSYLNWYQQKPGKAPKLLIYAASSLQSGVPSRFSGSGSGTDFTLTISSLQPEDFATYYCQQSKSTPFTFGQGTKLEIK

SEQ ID NO: 1015 - protein, 107 aa, synthetic construct
  source 1..107: note = 1A7[CD28]_L1.78
DIQMTQSPSSLSASVGDRVTITCRASQSISSYLNWYQQKPGKAPKLLIYAASSLQSGVPSRFSGSGSGTDFTLTISSLQPEDFATYYCQQSLSTPFTFGQGTKLEIK

SEQ ID NO: 1016 - protein, 107 aa, synthetic construct
  source 1..107: note = 1A7[CD28]_L1.79
DIQMTQSPSSLSASVGDRVTITCRASQSISSYLNWYQQKPGKAPKLLIYAASSLQSGVPSRFSGSGSGTDFTLTISSLQPEDFATYYCQQSQSTPFTFGQGTKLEIK

SEQ ID NO: 1017 - protein, 107 aa, synthetic construct
  source 1..107: note = 1A7[CD28]_L1.80
DIQMTQSPSSLSASVGDRVTITCRASQSISSYLNWYQQKPGKAPKLLIYAASSLQSGVPSRFSGSGSGTDFTLTISSLQPEDFATYYCQQSVSTPFTFGQGTKLEIK

SEQ ID NO: 1018 - protein, 107 aa, synthetic construct
  source 1..107: note = 1A7[CD28]_L1.81
DIQMTQSPSSLSASVGDRVTITCRASQSISSYLNWYQQKPGKAPKLLIYAASSLQSGVPSRFSGSGSGTDFTLTISSLQPEDFATYYCQQSWSTPFTFGQGTKLEIK

SEQ ID NO: 1019 - protein, 107 aa, synthetic construct
  source 1..107: note = 1A7[CD28]_L1.82
DIQMTQSPSSLSASVGDRVTITCRASQSISSYLNWYQQKPGKAPKLLIYAASSLQSGVPSRFSGSGSGTDFTLTISSLQPEDFATYYCQQSYATPFTFGQGTKLEIK

SEQ ID NO: 1020 - protein, 107 aa, synthetic construct
  source 1..107: note = 1A7[CD28]_L1.83
DIQMTQSPSSLSASVGDRVTITCRASQSISSYLNWYQQKPGKAPKLLIYAASSLQSGVPSRFSGSGSGTDFTLTISSLQPEDFATYYCQQSYDTPFTFGQGTKLEIK

SEQ ID NO: 1021 - protein, 107 aa, synthetic construct
  source 1..107: note = 1A7[CD28]_L1.84
DIQMTQSPSSLSASVGDRVTITCRASQSISSYLNWYQQKPGKAPKLLIYAASSLQSGVPSRFSGSGSGTDFTLTISSLQPEDFATYYCQQSYGTPFTFGQGTKLEIK

SEQ ID NO: 1022 - protein, 107 aa, synthetic construct
  source 1..107: note = 1A7[CD28]_L1.85
DIQMTQSPSSLSASVGDRVTITCRASQSISSYLNWYQQKPGKAPKLLIYAASSLQSGVPSRFSGSGSGTDFTLTISSLQPEDFATYYCQQSYHTPFTFGQGTKLEIK

SEQ ID NO: 1023 - protein, 107 aa, synthetic construct
  source 1..107: note = 1A7[CD28]_L1.86
DIQMTQSPSSLSASVGDRVTITCRASQSISSYLNWYQQKPGKAPKLLIYAASSLQSGVPSRFSGSGSGTDFTLTISSLQPEDFATYYCQQSYKTPFTFGQGTKLEIK

SEQ ID NO: 1024 - protein, 107 aa, synthetic construct
  source 1..107: note = 1A7[CD28]_L1.87
DIQMTQSPSSLSASVGDRVTITCRASQSISSYLNWYQQKPGKAPKLLIYAASSLQSGVPSRFSGSGSGTDFTLTISSLQPEDFATYYCQQSYNTPFTFGQGTKLEIK

SEQ ID NO: 1025 - protein, 107 aa, synthetic construct
  source 1..107: note = 1A7[CD28]_L1.88
DIQMTQSPSSLSASVGDRVTITCRASQSISSYLNWYQQKPGKAPKLLIYAASSLQSGVPSRFSGSGSGTDFTLTISSLQPEDFATYYCQQSYQTPFTFGQGTKLEIK

SEQ ID NO: 1026 - protein, 107 aa, synthetic construct
  source 1..107: note = 1A7[CD28]_L1.89
DIQMTQSPSSLSASVGDRVTITCRASQSISSYLNWYQQKPGKAPKLLIYAASSLQSGVPSRFSGSGSGTDFTLTISSLQPEDFATYYCQQSYTTPFTFGQGTKLEIK

SEQ ID NO: 1027 - protein, 107 aa, synthetic construct
  source 1..107: note = 1A7[CD28]_L1.90
DIQMTQSPSSLSASVGDRVTITCRASQSISSYLNWYQQKPGKAPKLLIYAASSLQSGVPSRFSGSGSGTDFTLTISSLQPEDFATYYCQQSYVTPFTFGQGTKLEIK

SEQ ID NO: 1028 - protein, 107 aa, synthetic construct
  source 1..107: note = 1A7[CD28]_L1.91
DIQMTQSPSSLSASVGDRVTITCRASQSISSYLNWYQQKPGKAPKLLIYAASSLQSGVPSRFSGSGSGTDFTLTISSLQPEDFATYYCQQSYYTPFTFGQGTKLEIK

SEQ ID NO: 1029 - protein, 107 aa, synthetic construct
  source 1..107: note = 1A7[CD28]_L1.92
DIQMTQSPSSLSASVGDRVTITCRASQSISSYLNWYQQKPGKAPKLLIYAASSLQSGVPSRFSGSGSGTDFTLTISSLQPEDFATYYCQQSYSAPFTFGQGTKLEIK

SEQ ID NO: 1030 - protein, 107 aa, synthetic construct
  source 1..107: note = 1A7[CD28]_L1.93
DIQMTQSPSSLSASVGDRVTITCRASQSISSYLNWYQQKPGKAPKLLIYAASSLQSGVPSRFSGSGSGTDFTLTISSLQPEDFATYYCQQSYSDPFTFGQGTKLEIK

SEQ ID NO: 1031 - protein, 107 aa, synthetic construct
  source 1..107: note = 1A7[CD28]_L1.94
DIQMTQSPSSLSASVGDRVTITCRASQSISSYLNWYQQKPGKAPKLLIYAASSLQSGVPSRFSGSGSGTDFTLTISSLQPEDFATYYCQQSYSFPFTFGQGTKLEIK

SEQ ID NO: 1032 - protein, 107 aa, synthetic construct
  source 1..107: note = 1A7[CD28]_L1.95
DIQMTQSPSSLSASVGDRVTITCRASQSISSYLNWYQQKPGKAPKLLIYAASSLQSGVPSRFSGSGSGTDFTLTISSLQPEDFATYYCQQSYSIPFTFGQGTKLEIK

SEQ ID NO: 1033 - protein, 107 aa, synthetic construct
  source 1..107: note = 1A7[CD28]_L1.96
DIQMTQSPSSLSASVGDRVTITCRASQSISSYLNWYQQKPGKAPKLLIYAASSLQSGVPSRFSGSGSGTDFTLTISSLQPEDFATYYCQQSYSKPFTFGQGTKLEIK

SEQ ID NO: 1034 - protein, 107 aa, synthetic construct
  source 1..107: note = 1A7[CD28]_L1.97
DIQMTQSPSSLSASVGDRVTITCRASQSISSYLNWYQQKPGKAPKLLIYAASSLQSGVPSRFSGSGSGTDFTLTISSLQPEDFATYYCQQSYSLPFTFGQGTKLEIK

SEQ ID NO: 1035 - protein, 107 aa, synthetic construct
  source 1..107: note = 1A7[CD28]_L1.98
DIQMTQSPSSLSASVGDRVTITCRASQSISSYLNWYQQKPGKAPKLLIYAASSLQSGVPSRFSGSGSGTDFTLTISSLQPEDFATYYCQQSYSQPFTFGQGTKLEIK

SEQ ID NO: 1036 - protein, 107 aa, synthetic construct
  source 1..107: note = 1A7[CD28]_L1.99
DIQMTQSPSSLSASVGDRVTITCRASQSISSYLNWYQQKPGKAPKLLIYAASSLQSGVPSRFSGSGSGTDFTLTISSLQPEDFATYYCQQSYSSPFTFGQGTKLEIK

SEQ ID NO: 1037 - protein, 107 aa, synthetic construct
  source 1..107: note = 1A7[CD28]_L1.100
DIQMTQSPSSLSASVGDRVTITCRASQSISSYLNWYQQKPGKAPKLLIYAASSLQSGVPSRFSGSGSGTDFTLTISSLQPEDFATYYCQQSYSVPFTFGQGTKLEIK

SEQ ID NO: 1038 - protein, 107 aa, synthetic construct
  source 1..107: note = 1A7[CD28]_L1.101
DIQMTQSPSSLSASVGDRVTITCRASQSISSYLNWYQQKPGKAPKLLIYAASSLQSGVPSRFSGSGSGTDFTLTISSLQPEDFATYYCQQSYSYPFTFGQGTKLEIK

SEQ ID NO: 1039 - protein, 107 aa, synthetic construct
  source 1..107: note = 1A7[CD28]_L1.102
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTKLEIK

SEQ ID NO: 1040 - protein, 107 aa, synthetic construct
  source 1..107: note = 1A7[CD28]_L1.103
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKLEIK

SEQ ID NO: 1041 - protein, 107 aa, synthetic construct
  source 1..107: note = 1A7[CD28]_L1.104
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKLEIK

SEQ ID NO: 1042 - protein, 107 aa, synthetic construct
  source 1..107: note = 1A7[CD28]_L1.105
DIQMTQSPSSLSASVGDRVTITCRASQSISSYLTWYQQKPGKAPKLLIYAASSLQSGVPSRFSGSGSGTDFTLTISSLQPEDFATYYCQQVYSTPFTFGQGTKLEIK

SEQ ID NO: 1043 - protein, 107 aa, synthetic construct
  source 1..107: note = 1A7[CD28]_L1.106
DIQMTQSPSSLSASVGDRVTITCRASQSISSYLTWYQQKPGKAPKLLIYAASSLQSGVPSRFSGSGSGTDFTLTISSLQPEDFATYYCQQSYNTPFTFGQGTKLEIK

SEQ ID NO: 1044 - protein, 4 aa, synthetic construct
YMNM

SEQ ID NO: 1045 - protein, 5 aa, synthetic construct
GSGGS

SEQ ID NO: 1046 - protein, 5 aa, synthetic construct
GGGGS

SEQ ID NO: 1047 - protein, 4 aa, synthetic construct
GGGS

SEQ ID NO: 1048 - protein, 479 aa, synthetic construct; identical to SEQ ID NO: 554
  source 1..479: note = XENP37902 Chain 2 - 1A7[CD28]_H1.14_L1_scFv(GKPGS)4 _Fc(216)_IgG1_C220S/PVA_/S267K/S364K/E357Q

SEQ ID NO: 1049 - protein, 479 aa, synthetic construct; identical to SEQ ID NO: 557
  source 1..479: note = XENP37903 Chain 2 - 1A7[CD28]_H1.14_L1.71_scFv(GKPG S)4_Fc(216)_IgG1_C220S/PVA_/S267K/S364K/E357Q

(19) United States
(12) Patent Application Publication
(10) Pub. No.: US 2024/0218082 A1
(43) Pub. Date: Jul. 4, 2024

(54) ANTI-CD28 X ANTI-PSMA ANTIBODIES

(71) Applicants: Xencor, Inc., Pasadena, CA (US); Janssen Biotech, Inc., Horsham, PA (US)

(72) Inventors: John R. Desjarlais, Pasadena, CA (US); Gregory Moore, Azusa, CA (US); Michael Hedvat, Encino, CA (US); Juan Diaz, Anaheim Hills, CA (US); Veronica Gusti Zeng, Duarte, CA (US); Matthew Adam Dragovich, Rancho Cucamonga, CA (US); Joseph Erhardt, Sellersville, PA (US); Theresa McDevitt, Warminster, PA (US); Fouad Moussa, Allentown, PA (US); Pankaj Seth, Norristown, PA (US); Fei Shen, Collegeville, PA (US); Adam Zwolak, Bala Cynwyd, PA (US)

(21) Appl. No.: 18/440,860

(22) Filed: Feb. 13, 2024

Related U.S. Application Data
(63) Continuation of application No. 18/311,771, filed on May 3, 2023.
Application No. 18/311,771 is a continuation of application No. 18/172,987, filed on Feb. 22, 2023.
(60) Provisional application No. 63/313,233, filed on Feb. 23, 2022.

(51) Int. Cl. C07K 16/46; C07K 16/28; C07K 16/30

(52) U.S. Cl. CPC C07K 16/468; C07K 16/2818; C07K 16/3069; C07K 2317/31; C07K 2317/565; C07K 2317/622

(57) ABSTRACT

Provided herein are novel anti-CD28 x anti-PSMA antibodies and methods of using such antibodies for the treatment of PSMA-associated cancers. Subject anti-CD28 x anti-PSMA antibodies are capable of agonistically binding to CD28 costimulatory molecules on T cells and PSMA on tumor cells. Thus, such antibodies selectively enhance anti-tumor activity at tumor sites while minimizing peripheral toxicity. The subject antibodies provided herein are particularly useful in combination with other anti-cancer therapies (e.g., anti-CD3 x anti-PSMA antibodies) for the treatment of prostate cancers.

### PRIORITY CLAIM

This application is continuation of U.S. patent application Ser. No. 18/311,771, filed May 3, 2023, which is a continuation of U.S. patent application Ser. No. 18/172,987, filed Feb. 22, 2023, which claims priority to and benefit of U.S. Provisional Application No. 63/313,233, filed on Feb. 23, 2022, the contents of which are hereby incorporated by reference in their entirety.

### SEQUENCE LISTING

The instant application contains a Sequence Listing which has been submitted electronically in XML format and is hereby incorporated by reference in its entirety. Said XML copy, created on Feb. 22, 2023, is named 067461-5297-WO_SL.xml and is 1,295,846 bytes in size.

### BACKGROUND

Prostate cancer (PC) is one of the most prevalent cancers in men, and end stage (castration-resistant prostate cancer) has no curative treatment option. Prostate Specific Membrane Antigen (PSMA), a type II transmembrane protein with a large extracellular domain, has long generated interest as a therapeutic target. PSMA is highly overexpressed in PC compared to normal tissue, and its expression has been shown to correlate with malignancy. Previous attempts to target PSMA include antibody-based radiotherapy and antibody drug conjugates, which have shown some success but can be hampered by the inherent toxicity of the modality.

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. One particular approach for bispecific antibodies is to engineer a first binding domain which engages CD3 and a second binding domain which engages an antigen associated with or upregulated on cancer cells (e.g., PSMA) so that the bispecific antibody redirects CD3⁺ T cells to destroy the cancer cells.

TILs, however, lose their cytotoxic ability over time due to upregulation of inhibitory immune checkpoints. While checkpoint blockade has demonstrated increased clinical response rates relative to other treatment options, many patients still fail to achieve a response to checkpoint blockade. Engagement of costimulatory receptors on TILs could provide a positive signal capable of overcoming negative signals of immune checkpoints. Preclinical and clinical studies of agonistic costimulatory receptor antibodies have indeed demonstrated that agonism of costimulatory receptors can result in impressive anti-tumor responses, activating T cells to attack tumor cells.

It is also important for cancer therapy to enhance anti-tumor activity by specifically destroying tumor cells while minimizing peripheral toxicity. In this context, it is crucial that only T cells in the presence of the target tumor cells are provided a costimulatory signal. However, agonism of costimulatory receptors with monospecific full-length antibodies is likely nondiscriminatory with regards to TILs vs. peripheral T cells vs. autoantigen-reactive T cells that contribute to autoimmune toxicities. Thus, there remains a need for novel immune response enhancing compositions for the treatment of cancers, including PSMA-associated cancers.

### SUMMARY

Provided herein are novel anti-CD28 x anti-PSMA antibodies and methods of using such antibodies for the treatment of PSMA-associated cancers. Subject anti-CD28 x anti-PSMA antibodies are capable of agonistically binding to CD28 costimulatory molecules on T cells and PSMA on tumor cells. Thus, such antibodies selectively enhance anti-tumor activity at tumor sites while minimizing peripheral toxicity. The subject antibodies provided herein are particularly useful in combination with other anti-cancer therapies (e.g., anti-CD3 x anti-PSMA antibodies) for the treatment of prostate cancers.

In a first aspect, provided herein are heterodimeric anti-CD28 x anti-PSMA antibodies in the 1+1 Fab-scFv-Fc format. These antibodies comprise: a) a first monomer; b) a second monomer; and c) a light chain. The first monomer comprises: i) a single chain variable fragment (scFv); and ii) a first Fc domain, wherein the scFv is covalently attached to the N-terminus of the first Fc domain using a domain linker. The second monomer comprises, from N-terminal to C-terminal, a VH1-CH1-hinge-CH2-CH3, wherein VH1 is a first variable heavy domain and CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 and the VL1 together form a first antigen binding domain (ABD) and the VH2 and the VL2 together form a second ABD, Further, one of the first ABD and second ABD binds CD28 and the other of the first ABD and second ABD binds Prostate Specific Membrane Antigen (PSMA).

In some embodiments, the first ABD binds human CD28 and the second ABD binds PSMA. In certain embodiments, the first ABD binds PSMA and the second ABD binds human CD28.

In some embodiments, the scFv comprises, from N-terminal to C-terminal, VH2-scFv linker-VL2. In certain embodiments, the scFv comprises, from N-terminal to C-terminal, VL2-scFv linker-VH2.

In some embodiments, the first ABD binds to human PSMA, and VH1 and VL1 are selected from the following:
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:206; and (ii) a VL1 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:210; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:214; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:218; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:222; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:226; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:230; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:234; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:238; and (ii) a VL1 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:242; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:246; and (ii) a VL1 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:250; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:254; and (ii) a VL1 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:258; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:262; and (ii) a VL1 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:266; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:270; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:274; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:278; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:282; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:286; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:290; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:294; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:298; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:302; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:306; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:310; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:314; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:318; and (ii) a VL1 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:322; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:326; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:330; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:334; and (ii) a VL1 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:338.

In some embodiments, VH1 and VL1 are selected from the following:
(i) a VH1 comprising a vhCDR1 having an amino acid sequence of SEQ ID NO:207; a vhCDR2 having an amino acid sequence of SEQ ID NO:208; and a vhCDR3 having an amino acid sequence of SEQ ID NO:209; and (ii) a VL1 comprising a vlCDR1 having an amino acid sequence of SEQ ID NO:211, a vlCDR2 having an amino acid sequence of SEQ ID NO: 212, and a vlCDR3 having an amino acid sequence of SEQ ID NO:213; or
(i) a VH1 comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL1 comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221;
(i) a VH1 comprising a vhCDR1 having an amino acid sequence of SEQ ID NO:223; a vhCDR2 having an amino acid sequence of SEQ ID NO:224; and a vhCDR3 having an amino acid sequence of SEQ ID NO:225; and (ii) a VL1 comprising a vlCDR1 having an amino acid sequence of SEQ ID NO:227, a vlCDR2 having an amino acid sequence of SEQ ID NO: 228, and a vlCDR3 having an amino acid sequence of SEQ ID NO:229; or
(i) a VH1 comprising a vhCDR1 having an amino acid sequence of SEQ ID NO:231; a vhCDR2 having an amino acid sequence of SEQ ID NO:232; and a vhCDR3 having an amino acid sequence of SEQ ID NO:233; and (ii) a VL1 comprising a vlCDR1 having an amino acid sequence of SEQ ID NO:235, a vlCDR2 having an amino acid sequence of SEQ ID NO: 236, and a vlCDR3 having an amino acid sequence of SEQ ID NO:237; or
(i) a VH1 comprising a vhCDR1 having an amino acid sequence of SEQ ID NO:239; a vhCDR2 having an amino acid sequence of SEQ ID NO:240; and a vhCDR3 having an amino acid sequence of SEQ ID NO:241; and (ii) a VL1 comprising a vlCDR1 having an amino acid sequence of SEQ ID NO:243, a vlCDR2 having an amino acid sequence of SEQ ID NO: 244, and a vlCDR3 having an amino acid sequence of SEQ ID NO:245; or
(i) a VH1 comprising a vhCDR1 having an amino acid sequence of SEQ ID NO:247; a vhCDR2 having an amino acid sequence of SEQ ID NO:248; and a vhCDR3 having an amino acid sequence of SEQ ID NO:249; and (ii) a VL1 comprising a vlCDR1 having an amino acid sequence of SEQ ID NO:251, a vlCDR2 having an amino acid sequence of SEQ ID NO: 252, and a vlCDR3 having an amino acid sequence of SEQ ID NO:253; or
(i) a VH1 comprising a vhCDR1 having an amino acid sequence of SEQ ID NO:255; a vhCDR2 having an amino acid sequence of SEQ ID NO:256; and a vhCDR3 having an amino acid sequence of SEQ ID NO:257; and (ii) a VL1 comprising a vlCDR1 having an amino acid sequence of SEQ ID NO:259, a vlCDR2 having an amino acid sequence of SEQ ID NO: 260, and a vlCDR3 having an amino acid sequence of SEQ ID NO:261; or
(i) a VH1 comprising a vhCDR1 having an amino acid sequence of SEQ ID NO:263; a vhCDR2 having an amino acid sequence of SEQ ID NO:264; and a vhCDR3 having an amino acid sequence of SEQ ID NO:265; and (ii) a VL1 comprising a vlCDR1 having an amino acid sequence of SEQ ID NO:267, a vlCDR2 having an amino acid sequence of SEQ ID NO: 268, and a vlCDR3 having an amino acid sequence of SEQ ID NO:269; or
(i) a VH1 comprising a vhCDR1 having an amino acid sequence of SEQ ID NO:271; a vhCDR2 having an amino acid sequence of SEQ ID NO:212; and a vhCDR3 having an amino acid sequence of SEQ ID NO:273; and (ii) a VL1 comprising a vlCDR1 having an amino acid sequence of SEQ ID NO:275, a vlCDR2 having an amino acid sequence of SEQ ID NO: 276, and a vlCDR3 having an amino acid sequence of SEQ ID NO:277; or
(i) a VH1 comprising a vhCDR1 having an amino acid sequence of SEQ ID NO:279; a vhCDR2 having an amino acid sequence of SEQ ID NO:280; and a vhCDR3 having an amino acid sequence of SEQ ID NO:281; and (ii) a VL1 comprising a vlCDR1 having an amino acid sequence of SEQ ID NO:283, a vlCDR2 having an amino acid sequence of SEQ ID NO: 284, and a vlCDR3 having an amino acid sequence of SEQ ID NO:285; or
(i) a VH1 comprising a vhCDR1 having an amino acid sequence of SEQ ID NO:287; a vhCDR2 having an amino acid sequence of SEQ ID NO:288; and a vhCDR3 having an amino acid sequence of SEQ ID NO:289; and (ii) a VL1 comprising a vlCDR1 having an amino acid sequence of SEQ ID NO:291, a vlCDR2 having an amino acid sequence of SEQ ID NO: 292, and a vlCDR3 having an amino acid sequence of SEQ ID NO:293; or
(i) a VH1 comprising a vhCDR1 having an amino acid sequence of SEQ ID NO:295; a vhCDR2 having an amino acid sequence of SEQ ID NO:296; and a vhCDR3 having an amino acid sequence of SEQ ID NO:297; and (ii) a VL1 comprising a vlCDR1 having an amino acid sequence of SEQ ID NO:299, a vlCDR2 having an amino acid sequence of SEQ ID NO: 300, and a vlCDR3 having an amino acid sequence of SEQ ID NO:301; or
(i) a VH1 comprising a vhCDR1 having an amino acid sequence of SEQ ID NO:303; a vhCDR2 having an amino acid sequence of SEQ ID NO:304; and a vhCDR3 having an amino acid sequence of SEQ ID NO:305; and (ii) a VL1 comprising a vlCDR1 having an amino acid sequence of SEQ ID NO:307, a vlCDR2 having an amino acid sequence of SEQ ID NO: 308, and a vlCDR3 having an amino acid sequence of SEQ ID NO:309; or
(i) a VH1 comprising a vhCDR1 having an amino acid sequence of SEQ ID NO:311; a vhCDR2 having an amino acid sequence of SEQ ID NO:312; and a vhCDR3 having an amino acid sequence of SEQ ID NO:313; and (ii) a VL1 comprising a vlCDR1 having an amino acid sequence of SEQ ID NO:315, a vlCDR2 having an amino acid sequence of SEQ ID NO: 316, and a vlCDR3 having an amino acid sequence of SEQ ID NO:317; or
(i) a VH1 comprising a vhCDR1 having an amino acid sequence of SEQ ID NO:319; a vhCDR2 having an amino acid sequence of SEQ ID NO:320; and a vhCDR3 having an amino acid sequence of SEQ ID NO:321; and (ii) a VL1 comprising a vlCDR1 having an amino acid sequence of SEQ ID NO:323, a vlCDR2 having an amino acid sequence of SEQ ID NO: 324, and a vlCDR3 having an amino acid sequence of SEQ ID NO:325; or
(i) a VH1 comprising a vhCDR1 having an amino acid sequence of SEQ ID NO:327; a vhCDR2 having an amino acid sequence of SEQ ID NO:328; and a vhCDR3 having an amino acid sequence of SEQ ID NO:329; and (ii) a VL1 comprising a vlCDR1 having an amino acid sequence of SEQ ID NO:331, a vlCDR2 having an amino acid sequence of SEQ ID NO: 332, and a vlCDR3 having an amino acid sequence of SEQ ID NO:333; or
(i) a VH1 comprising a vhCDR1 having an amino acid sequence of SEQ ID NO:335; a vhCDR2 having an amino acid sequence of SEQ ID NO:336; and a vhCDR3 having an amino acid sequence of SEQ ID NO:337; and (ii) a VL1 comprising a vlCDR1 having an amino acid sequence of SEQ ID NO:339, a vlCDR2 having an amino acid sequence of SEQ ID NO: 340, and a vlCDR3 having an amino acid sequence of SEQ ID NO:341.

In exemplary embodiments, VH1 and VL1 are selected from the following:
(i) a VH1 having an amino acid sequence of SEQ ID NO:206; and (ii) a VL1 having an amino acid sequence of SEQ ID NO:210; or
(i) a VH1 having an amino acid sequence of SEQ ID NO:214; and (ii) a VL1 having an amino acid sequence of SEQ ID NO:218; or
(i) a VH1 having an amino acid sequence of SEQ ID NO:222; and (ii) a VL1 having an amino acid sequence of SEQ ID NO:226; or
(i) a VH1 having an amino acid sequence of SEQ ID NO:230; and (ii) a VL1 having an amino acid sequence of SEQ ID NO:234; or
(i) a VH1 having an amino acid sequence of SEQ ID NO:238; and (ii) a VL1 having an amino acid sequence of SEQ ID NO:242; or
(i) a VH1 having an amino acid sequence of SEQ ID NO:246; and (ii) a VL1 having an amino acid sequence of SEQ ID NO:250; or
(i) a VH1 having an amino acid sequence of SEQ ID NO:254; and (ii) a VL1 having an amino acid sequence of SEQ ID NO:258; or
(i) a VH1 having an amino acid sequence of SEQ ID NO:262; and (ii) a VL1 having an amino acid sequence of SEQ ID NO:266; or
(i) a VH1 having an amino acid sequence of SEQ ID NO:270; and (ii) a VL1 having an amino acid sequence of SEQ ID NO:274; or
(i) a VH1 having an amino acid sequence of SEQ ID NO:278; and (ii) a VL1 having an amino acid sequence of SEQ ID NO:282; or
(i) a VH1 having an amino acid sequence of SEQ ID NO:286; and (ii) a VL1 having an amino acid sequence of SEQ ID NO:290; or
(i) a VH1 having an amino acid sequence of SEQ ID NO:294; and (ii) a VL1 having an amino acid sequence of SEQ ID NO:298; or
(i) a VH1 having an amino acid sequence of SEQ ID NO:302; and (ii) a VL1 having an amino acid sequence of SEQ ID NO:306; or
(i) a VH1 having an amino acid sequence of SEQ ID NO:310; and (ii) a VL1 having an amino acid sequence of SEQ ID NO:314; or
(i) a VH1 having an amino acid sequence of SEQ ID NO:318; and (ii) a VL1 having an amino acid sequence of SEQ ID NO:322; or
(i) a VH1 having an amino acid sequence of SEQ ID NO:326; and (ii) a VL1 having an amino acid sequence of SEQ ID NO:330; or
(i) a VH1 having an amino acid sequence of SEQ ID NO:334; and (ii) a VL1 having an amino acid sequence of SEQ ID NO:338.

In some embodiments, the second ABD binds to human CD28, and VH2 and VL2 are selected from the following:
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:13; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:31; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:35; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:43; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:47; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:51; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:55; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:59; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:67; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:90; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:94; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:98; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 102; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:106; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:110; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:114; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 118; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:122; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 126; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 130; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 134; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO: 138; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:142; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO: 146; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 150; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:158; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:166; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:170; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 174; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:178; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:182; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:186; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 190; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:194; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 198; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:202; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:380; and (ii) a VL2 comprising a vlCDR1, a vlCDR2, and a vICDR3 having an amino acid sequence of a vlCDR1, a vlCDR2, and a vlCDR3, respectively, of a VL having an amino acid sequence of SEQ ID NO:384; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:396; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:400; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:404; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:408; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:412; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:416; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:420; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:424.

In certain embodiments, VH2 and VL2 are selected from the following:
(i) a VH2 comprising a vhCDR1 having an amino acid sequence of SEQ ID NO: 10; a vhCDR2 having an amino acid sequence of SEQ ID NO: 11; and a vhCDR3 having an amino acid sequence of SEQ ID NO:12; and (ii) a VL2 comprising a vlCDR1 having an amino acid sequence of SEQ ID NO:14, a vlCDR2 having an amino acid sequence of SEQ ID NO: 15, and a vlCDR3 having an amino acid sequence of SEQ ID NO:16; or
(i) a VH2 comprising a vhCDR1 having an amino acid sequence of SEQ ID NO:32; a vhCDR2 having an amino acid sequence of SEQ ID NO:33; and a vhCDR3 having an amino acid sequence of SEQ ID NO:34; and (ii) a VL2 comprising a vlCDR1 having an amino acid sequence of SEQ ID NO:36, a vlCDR2 having an amino acid sequence of SEQ ID NO: 37, and a vlCDR3 having an amino acid sequence of SEQ ID NO:38; or
i) a VH2 comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL2 comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; or
i) a VH2 comprising a vhCDR1 having an amino acid sequence of SEQ ID NO:48; a vhCDR2 having an amino acid sequence of SEQ ID NO:49; and a vhCDR3 having an amino acid sequence of SEQ ID NO:50; and (ii) a VL2 comprising a vlCDR1 having an amino acid sequence of SEQ ID NO:52, a vlCDR2 having an amino acid sequence of SEQ ID NO: 53, and a vlCDR3 having an amino acid sequence of SEQ ID NO:54; or
i) a VH2 comprising a vhCDR1 having an amino acid sequence of SEQ ID NO:56; a vhCDR2 having an amino acid sequence of SEQ ID NO:57; and a vhCDR3 having an amino acid sequence of SEQ ID NO:58; and (ii) a VL2 comprising a vlCDR1 having an amino acid sequence of SEQ ID NO:60, a vlCDR2 having an amino acid sequence of SEQ ID NO:61, and a vlCDR3 having an amino acid sequence of SEQ ID NO:62; or
i) a VH2 comprising a vhCDR1 having an amino acid sequence of SEQ ID NO:64; a vhCDR2 having an amino acid sequence of SEQ ID NO:65; and a vhCDR3 having an amino acid sequence of SEQ ID NO:66; and (ii) a VL2 comprising a vlCDR1 having an amino acid sequence of SEQ ID NO:68, a vlCDR2 having an amino acid sequence of SEQ ID NO:69, and a vlCDR3 having an amino acid sequence of SEQ ID NO:70; or
i) a VH2 comprising a vhCDR1 having an amino acid sequence of SEQ ID NO:87; a vhCDR2 having an amino acid sequence of SEQ ID NO:88; and a vhCDR3 having an amino acid sequence of SEQ ID NO:89; and (ii) a VL2 comprising a vlCDR1 having an amino acid sequence of SEQ ID NO:91, a vlCDR2 having an amino acid sequence of SEQ ID NO:92, and a vlCDR3 having an amino acid sequence of SEQ ID NO:93; or
i) a VH2 comprising a vhCDR1 having an amino acid sequence of SEQ ID NO:95; a vhCDR2 having an amino acid sequence of SEQ ID NO:96; and a vhCDR3 having an amino acid sequence of SEQ ID NO:97; and (ii) a VL2 comprising a vlCDR1 having an amino acid sequence of SEQ ID NO:99, a vlCDR2 having an amino acid sequence of SEQ ID NO:100, and a vlCDR3 having an amino acid sequence of SEQ ID NO: 101; or
i) a VH2 comprising a vhCDR1 having an amino acid sequence of SEQ ID NO:103; a vhCDR2 having an amino acid sequence of SEQ ID NO: 104; and a vhCDR3 having an amino acid sequence of SEQ ID NO: 105; and (ii) a VL2 comprising a vlCDR1 having an amino acid sequence of SEQ ID NO: 107, a vlCDR2 having an amino acid sequence of SEQ ID NO: 108, and a vlCDR3 having an amino acid sequence of SEQ ID NO:109; or
i) a VH2 comprising a vhCDR1 having an amino acid sequence of SEQ ID NO:111; a vhCDR2 having an amino acid sequence of SEQ ID NO:112; and a vhCDR3 having an amino acid sequence of SEQ ID NO:113; and (ii) a VL2 comprising a vlCDR1 having an amino acid sequence of SEQ ID NO:115, a vlCDR2 having an amino acid sequence of SEQ ID NO:116, and a vlCDR3 having an amino acid sequence of SEQ ID NO:117; or
i) a VH2 comprising a vhCDR1 having an amino acid sequence of SEQ ID NO:119; a vhCDR2 having an amino acid sequence of SEQ ID NO:120; and a vhCDR3 having an amino acid sequence of SEQ ID NO:121; and (ii) a VL2 comprising a vlCDR1 having an amino acid sequence of SEQ ID NO: 123, a vlCDR2 having an amino acid sequence of SEQ ID NO: 124, and a vlCDR3 having an amino acid sequence of SEQ ID NO:125; or
i) a VH2 comprising a vhCDR1 having an amino acid sequence of SEQ ID NO:127; a vhCDR2 having an amino acid sequence of SEQ ID NO:128; and a vhCDR3 having an amino acid sequence of SEQ ID NO: 129; and (ii) a VL2 comprising a vlCDR1 having an amino acid sequence of SEQ ID NO: 131, a vlCDR2 having an amino acid sequence of SEQ ID NO: 132, and a vlCDR3 having an amino acid sequence of SEQ ID NO:133; or
i) a VH2 comprising a vhCDR1 having an amino acid sequence of SEQ ID NO:135; a vhCDR2 having an amino acid sequence of SEQ ID NO:136; and a vhCDR3 having an amino acid sequence of SEQ ID NO: 137; and (ii) a VL2 comprising a vlCDR1 having an amino acid sequence of SEQ ID NO:139, a vlCDR2 having an amino acid sequence of SEQ ID NO: 140, and a vlCDR3 having an amino acid sequence of SEQ ID NO:141; or
i) a VH2 comprising a vhCDR1 having an amino acid sequence of SEQ ID NO:143; a vhCDR2 having an amino acid sequence of SEQ ID NO:144; and a vhCDR3 having an amino acid sequence of SEQ ID NO:115; and (ii) a VL2 comprising a vlCDR1 having an amino acid sequence of SEQ ID NO: 147, a vlCDR2 having an amino acid sequence of SEQ ID NO:149, and a vlCDR3 having an amino acid sequence of SEQ ID NO: 149; or
i) a VH2 comprising a vhCDR1 having an amino acid sequence of SEQ ID NO:151; a vhCDR2 having an amino acid sequence of SEQ ID NO:152; and a vhCDR3 having an amino acid sequence of SEQ ID NO: 153; and (ii) a VL2 comprising a vlCDR1 having an amino acid sequence of SEQ ID NO:155, a vlCDR2 having an amino acid sequence of SEQ ID NO:156, and a vlCDR3 having an amino acid sequence of SEQ ID NO:157; or
i) a VH2 comprising a vhCDR1 having an amino acid sequence of SEQ ID NO:159; a vhCDR2 having an amino acid sequence of SEQ ID NO:160; and a vhCDR3 having an amino acid sequence of SEQ ID NO:161; and (ii) a VL2 comprising a vlCDR1 having an amino acid sequence of SEQ ID NO:163, a vlCDR2 having an amino acid sequence of SEQ ID NO:164, and a vlCDR3 having an amino acid sequence of SEQ ID NO:165; or
i) a VH2 comprising a vhCDR1 having an amino acid sequence of SEQ ID NO:167; a vhCDR2 having an amino acid sequence of SEQ ID NO:168; and a vhCDR3 having an amino acid sequence of SEQ ID NO: 169; and (ii) a VL2 comprising a vlCDR1 having an amino acid sequence of SEQ ID NO:171, a vlCDR2 having an amino acid sequence of SEQ ID NO: 172, and a vlCDR3 having an amino acid sequence of SEQ ID NO:173; or
i) a VH2 comprising a vhCDR1 having an amino acid sequence of SEQ ID NO:175; a vhCDR2 having an amino acid sequence of SEQ ID NO:176; and a vhCDR3 having an amino acid sequence of SEQ ID NO:177; and (ii) a VL2 comprising a vlCDR1 having an amino acid sequence of SEQ ID NO: 179, a vlCDR2 having an amino acid sequence of SEQ ID NO: 180, and a vlCDR3 having an amino acid sequence of SEQ ID NO:181; or
i) a VH2 comprising a vhCDR1 having an amino acid sequence of SEQ ID NO:183; a vhCDR2 having an amino acid sequence of SEQ ID NO:184; and a vhCDR3 having an amino acid sequence of SEQ ID NO: 185; and (ii) a VL2 comprising a vlCDR1 having an amino acid sequence of SEQ ID NO: 187, a vlCDR2 having an amino acid sequence of SEQ ID NO:188, and a vlCDR3 having an amino acid sequence of SEQ ID NO:189; or
i) a VH2 comprising a vhCDR1 having an amino acid sequence of SEQ ID NO:191; a vhCDR2 having an amino acid sequence of SEQ ID NO:192; and a vhCDR3 having an amino acid sequence of SEQ ID NO: 193; and (ii) a VL2 comprising a vlCDR1 having an amino acid sequence of SEQ ID NO:195, a vlCDR2 having an amino acid sequence of SEQ ID NO: 196, and a vlCDR3 having an amino acid sequence of SEQ ID NO:197; or
i) a VH2 comprising a vhCDR1 having an amino acid sequence of SEQ ID NO: 199; a vhCDR2 having an amino acid sequence of SEQ ID NO:200; and a vhCDR3 having an amino acid sequence of SEQ ID NO:201; and (ii) a VL2 comprising a vlCDR1 having an amino acid sequence of SEQ ID NO:203, a vlCDR2 having an amino acid sequence of SEQ ID NO:204, and a vlCDR3 having an amino acid sequence of SEQ ID NO:205; or
i) a VH2 comprising a vhCDR1 having an amino acid sequence of SEQ ID NO:381; a vhCDR2 having an amino acid sequence of SEQ ID NO:382; and a vhCDR3 having an amino acid sequence of SEQ ID NO:383; and (ii) a VL2 comprising a vlCDR1 having an amino acid sequence of SEQ ID NO:385, a vlCDR2 having an amino acid sequence of SEQ ID NO:386, and a vlCDR3 having an amino acid sequence of SEQ ID NO:387; or
i) a VH2 comprising a vhCDR1 having an amino acid sequence of SEQ ID NO:389; a vhCDR2 having an amino acid sequence of SEQ ID NO:390; and a vhCDR3 having an amino acid sequence of SEQ ID NO:391; and (ii) a VL2 comprising a vlCDR1 having an amino acid sequence of SEQ ID NO:393, a vlCDR2 having an amino acid sequence of SEQ ID NO:394, and a vlCDR3 having an amino acid sequence of SEQ ID NO:395; or
i) a VH2 comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL2 comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403; or
i) a VH2 comprising a vhCDR1 having an amino acid sequence of SEQ ID NO:405; a vhCDR2 having an amino acid sequence of SEQ ID NO:406; and a vhCDR3 having an amino acid sequence of SEQ ID NO:407; and (ii) a VL2 comprising a vlCDR1 having an amino acid sequence of SEQ ID NO:409, a vlCDR2 having an amino acid sequence of SEQ ID NO:410, and a vlCDR3 having an amino acid sequence of SEQ ID NO:411; or
i) a VH2 comprising a vhCDR1 having an amino acid sequence of SEQ ID NO:413; a vhCDR2 having an amino acid sequence of SEQ ID NO:414; and a vhCDR3 having an amino acid sequence of SEQ ID NO:415; and (ii) a VL2 comprising a vlCDR1 having an amino acid sequence of SEQ ID NO:417, a vlCDR2 having an amino acid sequence of SEQ ID NO:418, and a vlCDR3 having an amino acid sequence of SEQ ID NO:419; or
i) a VH2 comprising a vhCDR1 having an amino acid sequence of SEQ ID NO:421; a vhCDR2 having an amino acid sequence of SEQ ID NO:422; and a vhCDR3 having an amino acid sequence of SEQ ID NO:423; and (ii) a VL2 comprising a vlCDR1 having an amino acid sequence of SEQ ID NO:425, a vlCDR2 having an amino acid sequence of SEQ ID NO:426, and a vlCDR3 having an amino acid sequence of SEQ ID NO:427.

In exemplary embodiments, VH2 and VL2 are selected from the following:
(i) a VH2 having an amino acid sequence of SEQ ID NO:9; and (ii) a VL2 having an amino acid sequence of SEQ ID NO:13; or
(i) a VH2 having an amino acid sequence of SEQ ID NO:31; and (ii) a VL2 having an amino acid sequence of SEQ ID NO:35; or
(i) a VH2 having an amino acid sequence of SEQ ID NO:39; and (ii) a VL2 having an amino acid sequence of SEQ ID NO:43; or
(i) a VH2 having an amino acid sequence of SEQ ID NO:47; and (ii) a VL2 having an amino acid sequence of SEQ ID NO:51; or
(i) a VH2 having an amino acid sequence of SEQ ID NO:55; and (ii) a VL2 having an amino acid sequence of SEQ ID NO:59; or
(i) a VH2 having an amino acid sequence of SEQ ID NO:63; and (ii) a VL2 having an amino acid sequence of SEQ ID NO:67; or
(i) a VH2 having an amino acid sequence of SEQ ID NO:86; and (ii) a VL2 having an amino acid sequence of SEQ ID NO:90; or
(i) a VH2 having an amino acid sequence of SEQ ID NO:94; and (ii) a VL2 having an amino acid sequence of SEQ ID NO:98; or
(i) a VH2 having an amino acid sequence of SEQ ID NO:102; and (ii) a VL2 having an amino acid sequence of SEQ ID NO: 106; or
(i) a VH2 having an amino acid sequence of SEQ ID NO:110; and (ii) a VL2 having an amino acid sequence of SEQ ID NO:114; or
(i) a VH2 having an amino acid sequence of SEQ ID NO:118; and (ii) a VL2 having an amino acid sequence of SEQ ID NO:122; or
(i) a VH2 having an amino acid sequence of SEQ ID NO:126; and (ii) a VL2 having an amino acid sequence of SEQ ID NO:130; or
(i) a VH2 having an amino acid sequence of SEQ ID NO:134; and (ii) a VL2 having an amino acid sequence of SEQ ID NO: 138; or
(i) a VH2 having an amino acid sequence of SEQ ID NO:142; and (ii) a VL2 having an amino acid sequence of SEQ ID NO:146; or
(i) a VH2 having an amino acid sequence of SEQ ID NO:150; and (ii) a VL2 having an amino acid sequence of SEQ ID NO:154; or
(i) a VH2 having an amino acid sequence of SEQ ID NO:158; and (ii) a VL2 having an amino acid sequence of SEQ ID NO: 162; or
(i) a VH2 having an amino acid sequence of SEQ ID NO:166; and (ii) a VL2 having an amino acid sequence of SEQ ID NO:170; or
(i) a VH2 having an amino acid sequence of SEQ ID NO:174; and (ii) a VL2 having an amino acid sequence of SEQ ID NO:178; or
(i) a VH2 having an amino acid sequence of SEQ ID NO:182; and (ii) a VL2 having an amino acid sequence of SEQ ID NO:186; or
(i) a VH2 having an amino acid sequence of SEQ ID NO:190; and (ii) a VL2 having an amino acid sequence of SEQ ID NO:194; or
(i) a VH2 having an amino acid sequence of SEQ ID NO:198; and (ii) a VL2 having an amino acid sequence of SEQ ID NO:202; or
(i) a VH2 having an amino acid sequence of SEQ ID NO:380; and (ii) a VL2 having an amino acid sequence of SEQ ID NO:384; or
(i) a VH2 having an amino acid sequence of SEQ ID NO:388; and (ii) a VL2 having an amino acid sequence of SEQ ID NO:392; or
(i) a VH2 having an amino acid sequence of SEQ ID NO:396; and (ii) a VL2 having an amino acid sequence of SEQ ID NO:400; or
(i) a VH2 having an amino acid sequence of SEQ ID NO:404; and (ii) a VL2 having an amino acid sequence of SEQ ID NO:408; or
(i) a VH2 having an amino acid sequence of SEQ ID NO:412; and (ii) a VL2 having an amino acid sequence of SEQ ID NO:416; or
(i) a VH2 having an amino acid sequence of SEQ ID NO:420; and (ii) a VL2 having an amino acid sequence of SEQ ID NO:424.

In some embodiments, VH1 comprises a vhCDR1, a vhCDR2, and a vhCDR3 having an amino acid sequence of a vhCDR1, a vhCDR2, and a vhCDR3, respectively, of a VH having an amino acid sequence of SEQ ID NO:214; VL1 comprises a vlCDR1, a vlCDR2, and a vlCDR3 having an amino acid sequence of a vlCDR1, a vlCDR2, and a vICDR3, respectively, of a VL having an amino acid sequence of SEQ ID NO:218; VH2 comprises a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and VL2 comprises a vlCDR1, a vlCDR2, and a vlCDR3 having an amino acid sequence of a vlCDR1, a vlCDR2, and a vlCDR3, respectively, of a VL having an amino acid sequence of SEQ ID NO:43.

In exemplary embodiments, VH1 comprises a vhCDR1 having an amino acid sequence of SEQ ID NO:215; a vhCDR2 having an amino acid sequence of SEQ ID NO:216; and a vhCDR3 having an amino acid sequence of SEQ ID NO:217; VL1 comprises a vlCDR1 having an amino acid sequence of SEQ ID NO:219, a vlCDR2 having an amino acid sequence of SEQ ID NO: 220, and a vlCDR3 having an amino acid sequence of SEQ ID NO:221, VH2 comprises a vhCDR1 having an amino acid sequence of SEQ ID NO:40; a vhCDR2 having an amino acid sequence of SEQ ID NO:41; and a vhCDR3 having an amino acid sequence of SEQ ID NO:42; and VL2 comprises a vlCDR1 having an amino acid sequence of SEQ ID NO:44, a vlCDR2 having an amino acid sequence of SEQ ID NO:45, and a vlCDR3 having an amino acid sequence of SEQ ID NO:46.

In some embodiments, the first Fc domain and second Fc domain are each variant Fc domains. In exemplary embodiments, one of the first and second Fc domains comprises heterodimerization variant T366W, and the other of the first and second Fc domains comprises heterodimerization variants T366S/L368A/Y407V, wherein numbering is according to EU numbering. In some embodiments, the first Fc domain comprises heterodimerization variant T366W, and the second Fc domain comprises heterodimerization variants T366S/L368A/Y407V.

In certain embodiments, the first and second Fc domains each comprise one or more ablation variants. In some embodiments, one or more ablation variants comprise L234A/L235A/D265S, wherein numbering is according to EU numbering.

In some embodiments, the first or second Fc domain comprises purification variants H435R/Y436F, wherein numbering is according to EU numbering.

In exemplary embodiments, the second Fc domain comprises purification variants H435R/Y436F.

In exemplary embodiments, the first Fc domain comprises amino acid substitutions L234A/L235A/D265S/LT366W, and the second Fc domain comprises amino acid substitutions L234A/L235A/D265S/T366S/L368A/Y407V/H435R/Y436F, wherein numbering is according to EU numbering. In some embodiments, the first and second Fc domains each further comprise amino acid substitutions M252Y/S254T/T256E, wherein numbering is according to EU numbering.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some emboidments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises a pI variant. In some embodiments, the CH1-hinge-CH2-CH3 of the second monomer comprises pI variants N208D/Q295E/N384D/Q418E/N421D, wherein numbering is according to EU numbering.

In some emboidments, the CH1-hinge-CH2-CH3 of the second monomer comprises amino acid variants

E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, of the anti-CD28 x anti-PSMA antibody, the scFv linker is selected from GKPGSGKPGSGKPGSGKPGS (SEQ ID NO: 443), GGSEGKSSGSGSESKSTGGS (SEQ ID NO: 456), and GGGSGGSGGCPPCGGSGG (SEQ ID NO: 457).

In a second aspect, provided herein are heterodimeric anti-CD28 x anti-PSMA antibodies in the 2+1 Fab2-scFv-Fc format. These antibodies comprise: a) a first monomer; b) a second monomer; and c) a light chain. The first monomer comprises, from N-terminal to C-terminal, VH1-CH1-first domain linker-scFv-second domain linker-CH2-CH3, wherein VH1 is a first variable heavy domain, and CH2-CH3 is a first Fc domain. The second monomer comprises, from N-terminal to C-terminal, a VH1-CH1-hinge-CH2-CH3, wherein CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). Each of the VH1s and the VL1 together form a first antigen binding domain (ABD), and the VH2 and the VL2 form a second ABD. Further, one of the first and second ABDs bind human CD28 and the other of the first and second ABDs binds PSMA.

In some embodiments, the first ABDs bind human CD28 and the second ABD binds PSMA. In certain embodiments, the first ABDs bind PSMA and the second ABD binds human CD28.

In some embodiments, the scFv comprises, from N-terminal to C-terminal, VH2-scFv linker-VL2. In certain embodiments, the scFv comprises, from N-terminal to C-terminal, VL2-scFv linker-VH2.

In some embodiments, the first ABDs bind to human PSMA, and VH1 and VL1 are selected from the following:
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:206; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:210; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:214; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:218; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:222; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:226; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:230; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:234; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:238; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:242; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:246; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:250; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:254; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:258; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:262; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:266; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:270; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:274; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:278; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:282; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:286; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:290; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:294; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:298; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:302; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:306; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:310; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:314; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:318; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:322; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:326; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:330; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:334; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:338.

In some embodiments, VH1 and VL1 are selected from the following:
(i) a VH1 comprising a vhCDR1 having an amino acid sequence of SEQ ID NO:207; a vhCDR2 having an amino acid sequence of SEQ ID NO:208; and a vhCDR3 having an amino acid sequence of SEQ ID NO:209; and (ii) a VL1 comprising a vlCDR1 having an amino acid sequence of SEQ ID NO:211, a vlCDR2 having an amino acid sequence of SEQ ID NO: 212, and a vlCDR3 having an amino acid sequence of SEQ ID NO:213; or
(i) a VH1 comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL1 comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221;
(i) a VH1 comprising a vhCDR1 having an amino acid sequence of SEQ ID NO:223; a vhCDR2 having an amino acid sequence of SEQ ID NO:224; and a vhCDR3 having an amino acid sequence of SEQ ID NO:225; and (ii) a VL1 comprising a vlCDR1 having an amino acid sequence of SEQ ID NO:227, a vlCDR2 having an amino acid sequence of SEQ ID NO: 228, and a vlCDR3 having an amino acid sequence of SEQ ID NO:229; or
(i) a VH1 comprising a vhCDR1 having an amino acid sequence of SEQ ID NO:231; a vhCDR2 having an amino acid sequence of SEQ ID NO:232; and a vhCDR3 having an amino acid sequence of SEQ ID NO:233; and (ii) a VL1 comprising a vlCDR1 having an amino acid sequence of SEQ ID NO:235, a vlCDR2 having an amino acid sequence of SEQ ID NO: 236, and a vlCDR3 having an amino acid sequence of SEQ ID NO:237; or
(i) a VH1 comprising a vhCDR1 having an amino acid sequence of SEQ ID NO:239; a vhCDR2 having an amino acid sequence of SEQ ID NO:240; and a vhCDR3 having an amino acid sequence of SEQ ID NO:241; and (ii) a VL1 comprising a vlCDR1 having an amino acid sequence of SEQ ID NO:243, a vlCDR2 having an amino acid sequence of SEQ ID NO: 244, and a vlCDR3 having an amino acid sequence of SEQ ID NO:245; or
(i) a VH1 comprising a vhCDR1 having an amino acid sequence of SEQ ID NO:247; a vhCDR2 having an amino acid sequence of SEQ ID NO:248; and a vhCDR3 having an amino acid sequence of SEQ ID NO:249; and (ii) a VL1 comprising a vlCDR1 having an amino acid sequence of SEQ ID NO:251, a vlCDR2 having an amino acid sequence of SEQ ID NO: 252, and a vlCDR3 having an amino acid sequence of SEQ ID NO:253; or
(i) a VH1 comprising a vhCDR1 having an amino acid sequence of SEQ ID NO:255; a vhCDR2 having an amino acid sequence of SEQ ID NO:256; and a vhCDR3 having an amino acid sequence of SEQ ID NO:257; and (ii) a VL1 comprising a vlCDR1 having an amino acid sequence of SEQ ID NO:259, a vlCDR2 having an amino acid sequence of SEQ ID NO: 260, and a vlCDR3 having an amino acid sequence of SEQ ID NO:261; or
(i) a VH1 comprising a vhCDR1 having an amino acid sequence of SEQ ID NO:263; a vhCDR2 having an amino acid sequence of SEQ ID NO:264; and a vhCDR3 having an amino acid sequence of SEQ ID NO:265; and (ii) a VL1 comprising a vlCDR1 having an amino acid sequence of SEQ ID NO:267, a vlCDR2 having an amino acid sequence of SEQ ID NO: 268, and a vlCDR3 having an amino acid sequence of SEQ ID NO:269; or
(i) a VH1 comprising a vhCDR1 having an amino acid sequence of SEQ ID NO:271; a vhCDR2 having an amino acid sequence of SEQ ID NO:212; and a vhCDR3 having an amino acid sequence of SEQ ID NO:273; and (ii) a VL1 comprising a vlCDR1 having an amino acid sequence of SEQ ID NO:275, a vlCDR2 having an amino acid sequence of SEQ ID NO: 276, and a vlCDR3 having an amino acid sequence of SEQ ID NO:277; or
(i) a VH1 comprising a vhCDR1 having an amino acid sequence of SEQ ID NO:279; a vhCDR2 having an amino acid sequence of SEQ ID NO:280; and a vhCDR3 having an amino acid sequence of SEQ ID NO:281; and (ii) a VL1 comprising a vlCDR1 having an amino acid sequence of SEQ ID NO:283, a vlCDR2 having an amino acid sequence of SEQ ID NO: 284, and a vlCDR3 having an amino acid sequence of SEQ ID NO:285; or
(i) a VH1 comprising a vhCDR1 having an amino acid sequence of SEQ ID NO:287; a vhCDR2 having an amino acid sequence of SEQ ID NO:288; and a vhCDR3 having an amino acid sequence of SEQ ID NO:289; and (ii) a VL1 comprising a vlCDR1 having an amino acid sequence of SEQ ID NO:291, a vlCDR2 having an amino acid sequence of SEQ ID NO: 292, and a vlCDR3 having an amino acid sequence of SEQ ID NO:293; or
(i) a VH1 comprising a vhCDR1 having an amino acid sequence of SEQ ID NO:295; a vhCDR2 having an amino acid sequence of SEQ ID NO:296; and a vhCDR3 having an amino acid sequence of SEQ ID NO:297; and (ii) a VL1 comprising a vlCDR1 having an amino acid sequence of SEQ ID NO:299, a vlCDR2 having an amino acid sequence of SEQ ID NO: 300, and a vlCDR3 having an amino acid sequence of SEQ ID NO:301; or
(i) a VH1 comprising a vhCDR1 having an amino acid sequence of SEQ ID NO:303; a vhCDR2 having an amino acid sequence of SEQ ID NO:304; and a vhCDR3 having an amino acid sequence of SEQ ID NO:305; and (ii) a VL1 comprising a vlCDR1 having an amino acid sequence of SEQ ID NO:307, a vlCDR2 having an amino acid sequence of SEQ ID NO: 308, and a vlCDR3 having an amino acid sequence of SEQ ID NO:309; or
(i) a VH1 comprising a vhCDR1 having an amino acid sequence of SEQ ID NO:311; a vhCDR2 having an amino acid sequence of SEQ ID NO:312; and a vhCDR3 having an amino acid sequence of SEQ ID NO:313; and (ii) a VL1 comprising a vlCDR1 having an amino acid sequence of SEQ ID NO:315, a vlCDR2 having an amino acid sequence of SEQ ID NO: 316, and a vlCDR3 having an amino acid sequence of SEQ ID NO:317; or
(i) a VH1 comprising a vhCDR1 having an amino acid sequence of SEQ ID NO:319; a vhCDR2 having an amino acid sequence of SEQ ID NO:320; and a vhCDR3 having an amino acid sequence of SEQ ID NO:321; and (ii) a VL1 comprising a vlCDR1 having an amino acid sequence of SEQ ID NO:323, a vlCDR2 having an amino acid sequence of SEQ ID NO: 324, and a vlCDR3 having an amino acid sequence of SEQ ID NO:325; or
(i) a VH1 comprising a vhCDR1 having an amino acid sequence of SEQ ID NO:327; a vhCDR2 having an amino acid sequence of SEQ ID NO:328; and a vhCDR3 having an amino acid sequence of SEQ ID NO:329; and (ii) a VL1 comprising a vlCDR1 having an amino acid sequence of SEQ ID NO:331, a vlCDR2 having an amino acid sequence of SEQ ID NO: 332, and a vlCDR3 having an amino acid sequence of SEQ ID NO:333; or
(i) a VH1 comprising a vhCDR1 having an amino acid sequence of SEQ ID NO:335; a vhCDR2 having an amino acid sequence of SEQ ID NO:336; and a vhCDR3 having an amino acid sequence of SEQ ID NO:337; and (ii) a VL1 comprising a vlCDR1 having an amino acid sequence of SEQ ID NO:339, a vlCDR2 having an amino acid sequence of SEQ ID NO: 340, and a vlCDR3 having an amino acid sequence of SEQ ID NO:341.

In exemplary embodiments, VH1 and VL1 are selected from the following:
(i) a VH1 having an amino acid sequence of SEQ ID NO:206; and (ii) a VL1 having an amino acid sequence of SEQ ID NO:210; or
(i) a VH1 having an amino acid sequence of SEQ ID NO:214; and (ii) a VL1 having an amino acid sequence of SEQ ID NO:218; or
(i) a VH1 having an amino acid sequence of SEQ ID NO:222; and (ii) a VL1 having an amino acid sequence of SEQ ID NO:226; or
(i) a VH1 having an amino acid sequence of SEQ ID NO:230; and (ii) a VL1 having an amino acid sequence of SEQ ID NO:234; or
(i) a VH1 having an amino acid sequence of SEQ ID NO:238; and (ii) a VL1 having an amino acid sequence of SEQ ID NO:242; or
(i) a VH1 having an amino acid sequence of SEQ ID NO:246; and (ii) a VL1 having an amino acid sequence of SEQ ID NO:250; or
(i) a VH1 having an amino acid sequence of SEQ ID NO:254; and (ii) a VL1 having an amino acid sequence of SEQ ID NO:258; or
(i) a VH1 having an amino acid sequence of SEQ ID NO:262; and (ii) a VL1 having an amino acid sequence of SEQ ID NO:266; or
(i) a VH1 having an amino acid sequence of SEQ ID NO:270; and (ii) a VL1 having an amino acid sequence of SEQ ID NO:274; or
(i) a VH1 having an amino acid sequence of SEQ ID NO:278; and (ii) a VL1 having an amino acid sequence of SEQ ID NO:282; or
(i) a VH1 having an amino acid sequence of SEQ ID NO:286; and (ii) a VL1 having an amino acid sequence of SEQ ID NO:290; or
(i) a VH1 having an amino acid sequence of SEQ ID NO:294; and (ii) a VL1 having an amino acid sequence of SEQ ID NO:298; or
(i) a VH1 having an amino acid sequence of SEQ ID NO:302; and (ii) a VL1 having an amino acid sequence of SEQ ID NO:306; or
(i) a VH1 having an amino acid sequence of SEQ ID NO:310; and (ii) a VL1 having an amino acid sequence of SEQ ID NO:314; or
(i) a VH1 having an amino acid sequence of SEQ ID NO:318; and (ii) a VL1 having an amino acid sequence of SEQ ID NO:322; or
(i) a VH1 having an amino acid sequence of SEQ ID NO:326; and (ii) a VL1 having an amino acid sequence of SEQ ID NO:330; or
(i) a VH1 having an amino acid sequence of SEQ ID NO:334; and (ii) a VL1 having an amino acid sequence of SEQ ID NO:338.

In some embodiments, the second ABD binds to human CD28, and VH2 and VL2 are selected from the following:
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:13; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:31; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:35; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:43; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:47; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:51; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:55; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:59; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:67; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:90; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:94; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:98; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 102; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 106; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:110; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 114; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:118; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:122; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 126; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:130; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:134; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:138; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:142; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 146; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:150; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 158; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 166; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:170; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:174; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:178; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 182; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 186; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 190; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 194; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 198; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:202; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:380; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:384; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:396; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:400; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:404; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:408; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:412; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:416; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:420; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:424.

In certain embodiments, VH2 and VL2 are selected from the following:
(i) a VH2 comprising a vhCDR1 having an amino acid sequence of SEQ ID NO:10; a vhCDR2 having an amino acid sequence of SEQ ID NO:11; and a vhCDR3 having an amino acid sequence of SEQ ID NO:12; and (ii) a VL2 comprising a vlCDR1 having an amino acid sequence of SEQ ID NO:14, a vlCDR2 having an amino acid sequence of SEQ ID NO: 15, and a vlCDR3 having an amino acid sequence of SEQ ID NO: 16; or
(i) a VH2 comprising a vhCDR1 having an amino acid sequence of SEQ ID NO:32; a vhCDR2 having an amino acid sequence of SEQ ID NO:33; and a vhCDR3 having an amino acid sequence of SEQ ID NO:34; and (ii) a VL2 comprising a vlCDR1 having an amino acid sequence of SEQ ID NO:36, a vlCDR2 having an amino acid sequence of SEQ ID NO: 37, and a vlCDR3 having an amino acid sequence of SEQ ID NO:38; or
i) a VH2 comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL2 comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; or
i) a VH2 comprising a vhCDR1 having an amino acid sequence of SEQ ID NO:48; a vhCDR2 having an amino acid sequence of SEQ ID NO:49; and a vhCDR3 having an amino acid sequence of SEQ ID NO:50; and (ii) a VL2 comprising a vlCDR1 having an amino acid sequence of SEQ ID NO:52, a vlCDR2 having an amino acid sequence of SEQ ID NO: 53, and a vlCDR3 having an amino acid sequence of SEQ ID NO:54; or
i) a VH2 comprising a vhCDR1 having an amino acid sequence of SEQ ID NO:56; a vhCDR2 having an amino acid sequence of SEQ ID NO:57; and a vhCDR3 having an amino acid sequence of SEQ ID NO:58; and (ii) a VL2 comprising a vlCDR1 having an amino acid sequence of SEQ ID NO:60, a vlCDR2 having an amino acid sequence of SEQ ID NO:61, and a vlCDR3 having an amino acid sequence of SEQ ID NO:62; or
i) a VH2 comprising a vhCDR1 having an amino acid sequence of SEQ ID NO:64; a vhCDR2 having an amino acid sequence of SEQ ID NO:65; and a vhCDR3 having an amino acid sequence of SEQ ID NO:66; and (ii) a VL2 comprising a vlCDR1 having an amino acid sequence of SEQ ID NO:68, a vlCDR2 having an amino acid sequence of SEQ ID NO:69, and a vlCDR3 having an amino acid sequence of SEQ ID NO:70; or
i) a VH2 comprising a vhCDR1 having an amino acid sequence of SEQ ID NO:87; a vhCDR2 having an amino acid sequence of SEQ ID NO:88; and a vhCDR3 having an amino acid sequence of SEQ ID NO:89; and (ii) a VL2 comprising a vlCDR1 having an amino acid sequence of SEQ ID NO:91, a vlCDR2 having an amino acid sequence of SEQ ID NO:92, and a vlCDR3 having an amino acid sequence of SEQ ID NO:93; or
i) a VH2 comprising a vhCDR1 having an amino acid sequence of SEQ ID NO:95; a vhCDR2 having an amino acid sequence of SEQ ID NO:96; and a vhCDR3 having an amino acid sequence of SEQ ID NO:97; and (ii) a VL2 comprising a vlCDR1 having an amino acid sequence of SEQ ID NO:99, a vlCDR2 having an amino acid sequence of SEQ ID NO:100, and a vlCDR3 having an amino acid sequence of SEQ ID NO:101; or
i) a VH2 comprising a vhCDR1 having an amino acid sequence of SEQ ID NO:103; a vhCDR2 having an amino acid sequence of SEQ ID NO:104; and a vhCDR3 having an amino acid sequence of SEQ ID NO: 105; and (ii) a VL2 comprising a vlCDR1 having an amino acid sequence of SEQ ID NO: 107, a vlCDR2 having an amino acid sequence of SEQ ID NO: 108, and a vlCDR3 having an amino acid sequence of SEQ ID NO: 109; or
i) a VH2 comprising a vhCDR1 having an amino acid sequence of SEQ ID NO:111; a vhCDR2 having an amino acid sequence of SEQ ID NO:112; and a vhCDR3 having an amino acid sequence of SEQ ID NO:113; and (ii) a VL2 comprising a vlCDR1 having an amino acid sequence of SEQ ID NO:115, a vlCDR2 having an amino acid sequence of SEQ ID NO:116, and a vlCDR3 having an amino acid sequence of SEQ ID NO:117; or
i) a VH2 comprising a vhCDR1 having an amino acid sequence of SEQ ID NO:119; a vhCDR2 having an amino acid sequence of SEQ ID NO:120; and a vhCDR3 having an amino acid sequence of SEQ ID NO: 121; and (ii) a VL2 comprising a vlCDR1 having an amino acid sequence of SEQ ID NO:123, a vlCDR2 having an amino acid sequence of SEQ ID NO:124, and a vlCDR3 having an amino acid sequence of SEQ ID NO:125; or
i) a VH2 comprising a vhCDR1 having an amino acid sequence of SEQ ID NO:127; a vhCDR2 having an amino acid sequence of SEQ ID NO:128; and a vhCDR3 having an amino acid sequence of SEQ ID NO: 129; and (ii) a VL2 comprising a vlCDR1 having an amino acid sequence of SEQ ID NO: 131, a vlCDR2 having an amino acid sequence of SEQ ID NO: 132, and a vlCDR3 having an amino acid sequence of SEQ ID NO:133; or
i) a VH2 comprising a vhCDR1 having an amino acid sequence of SEQ ID NO:135; a vhCDR2 having an amino acid sequence of SEQ ID NO:136; and a vhCDR3 having an amino acid sequence of SEQ ID NO: 137; and (ii) a VL2 comprising a vlCDR1 having an amino acid sequence of SEQ ID NO: 139, a vlCDR2 having an amino acid sequence of SEQ ID NO: 140, and a vlCDR3 having an amino acid sequence of SEQ ID NO:141; or
i) a VH2 comprising a vhCDR1 having an amino acid sequence of SEQ ID NO:143; a vhCDR2 having an amino acid sequence of SEQ ID NO:144; and a vhCDR3 having an amino acid sequence of SEQ ID NO:115; and (ii) a VL2 comprising a vlCDR1 having an amino acid sequence of SEQ ID NO: 147, a vlCDR2 having an amino acid sequence of SEQ ID NO:149, and a vlCDR3 having an amino acid sequence of SEQ ID NO:149; or
i) a VH2 comprising a vhCDR1 having an amino acid sequence of SEQ ID NO:151; a vhCDR2 having an amino acid sequence of SEQ ID NO:152; and a vhCDR3 having an amino acid sequence of SEQ ID NO: 153; and (ii) a VL2 comprising a vlCDR1 having an amino acid sequence of SEQ ID NO: 155, a vlCDR2 having an amino acid sequence of SEQ ID NO:156, and a vlCDR3 having an amino acid sequence of SEQ ID NO:157; or
i) a VH2 comprising a vhCDR1 having an amino acid sequence of SEQ ID NO:159; a vhCDR2 having an amino acid sequence of SEQ ID NO:160; and a vhCDR3 having an amino acid sequence of SEQ ID NO: 161; and (ii) a VL2 comprising a vlCDR1 having an amino acid sequence of SEQ ID NO: 163, a vlCDR2 having an amino acid sequence of SEQ ID NO: 164, and a vlCDR3 having an amino acid sequence of SEQ ID NO:165; or
i) a VH2 comprising a vhCDR1 having an amino acid sequence of SEQ ID NO:167; a vhCDR2 having an amino acid sequence of SEQ ID NO:168; and a vhCDR3 having an amino acid sequence of SEQ ID NO:169; and (ii) a VL2 comprising a vlCDR1 having an amino acid sequence of SEQ ID NO: 171, a vlCDR2 having an amino acid sequence of SEQ ID NO: 172, and a vlCDR3 having an amino acid sequence of SEQ ID NO:173; or
i) a VH2 comprising a vhCDR1 having an amino acid sequence of SEQ ID NO:175; a vhCDR2 having an amino acid sequence of SEQ ID NO:176; and a vhCDR3 having an amino acid sequence of SEQ ID NO:177; and (ii) a VL2 comprising a vlCDR1 having an amino acid sequence of SEQ ID NO: 179, a vlCDR2 having an amino acid sequence of SEQ ID NO:180, and a vlCDR3 having an amino acid sequence of SEQ ID NO:181; or
i) a VH2 comprising a vhCDR1 having an amino acid sequence of SEQ ID NO:183; a vhCDR2 having an amino acid sequence of SEQ ID NO:184; and a vhCDR3 having an amino acid sequence of SEQ ID NO:185; and (ii) a VL2 comprising a vlCDR1 having an amino acid sequence of SEQ ID NO:187, a vlCDR2 having an amino acid sequence of SEQ ID NO:188, and a vlCDR3 having an amino acid sequence of SEQ ID NO:189; or
i) a VH2 comprising a vhCDR1 having an amino acid sequence of SEQ ID NO:191; a vhCDR2 having an amino acid sequence of SEQ ID NO:192; and a vhCDR3 having an amino acid sequence of SEQ ID NO:193; and (ii) a VL2 comprising a vlCDR1 having an amino acid sequence of SEQ ID NO:195, a vlCDR2 having an amino acid sequence of SEQ ID NO: 196, and a vlCDR3 having an amino acid sequence of SEQ ID NO:197; or
i) a VH2 comprising a vhCDR1 having an amino acid sequence of SEQ ID NO:199; a vhCDR2 having an amino acid sequence of SEQ ID NO:200; and a vhCDR3 having an amino acid sequence of SEQ ID NO:201; and (ii) a VL2 comprising a vlCDR1 having an amino acid sequence of SEQ ID NO:203, a vlCDR2 having an amino acid sequence of SEQ ID NO:204, and a vlCDR3 having an amino acid sequence of SEQ ID NO:205; or
i) a VH2 comprising a vhCDR1 having an amino acid sequence of SEQ ID NO:381; a vhCDR2 having an amino acid sequence of SEQ ID NO:382; and a vhCDR3 having an amino acid sequence of SEQ ID NO:383; and (ii) a VL2 comprising a vlCDR1 having an amino acid sequence of SEQ ID NO:385, a vlCDR2 having an amino acid sequence of SEQ ID NO:386, and a vlCDR3 having an amino acid sequence of SEQ ID NO:387; or
i) a VH2 comprising a vhCDR1 having an amino acid sequence of SEQ ID NO:389; a vhCDR2 having an amino acid sequence of SEQ ID NO:390; and a vhCDR3 having an amino acid sequence of SEQ ID NO:391; and (ii) a VL2 comprising a vlCDR1 having an amino acid sequence of SEQ ID NO:393, a vlCDR2 having an amino acid sequence of SEQ ID NO:394, and a vlCDR3 having an amino acid sequence of SEQ ID NO:395; or
i) a VH2 comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL2 comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403; or
i) a VH2 comprising a vhCDR1 having an amino acid sequence of SEQ ID NO:405; a vhCDR2 having an amino acid sequence of SEQ ID NO:406; and a vhCDR3 having an amino acid sequence of SEQ ID NO:407; and (ii) a VL2 comprising a vlCDR1 having an amino acid sequence of SEQ ID NO:409, a vlCDR2 having an amino acid sequence of SEQ ID NO:410, and a vlCDR3 having an amino acid sequence of SEQ ID NO:411; or
i) a VH2 comprising a vhCDR1 having an amino acid sequence of SEQ ID NO:413; a vhCDR2 having an amino acid sequence of SEQ ID NO:414; and a vhCDR3 having an amino acid sequence of SEQ ID NO:415; and (ii) a VL2 comprising a vlCDR1 having an amino acid sequence of SEQ ID NO:417, a vlCDR2 having an amino acid sequence of SEQ ID NO:418, and a vlCDR3 having an amino acid sequence of SEQ ID NO:419; or
i) a VH2 comprising a vhCDR1 having an amino acid sequence of SEQ ID NO:421; a vhCDR2 having an amino acid sequence of SEQ ID NO:422; and a vhCDR3 having an amino acid sequence of SEQ ID NO:423; and (ii) a VL2 comprising a vlCDR1 having an amino acid sequence of SEQ ID NO:425, a vlCDR2 having an amino acid sequence of SEQ ID NO:426, and a vlCDR3 having an amino acid sequence of SEQ ID NO:427.

In exemplary embodiments, VH2 and VL2 are selected from the following:
(i) a VH2 having an amino acid sequence of SEQ ID NO:9; and (ii) a VL2 having an amino acid sequence of SEQ ID NO:13; or
(i) a VH2 having an amino acid sequence of SEQ ID NO:31; and (ii) a VL2 having an amino acid sequence of SEQ ID NO:35; or
(i) a VH2 having an amino acid sequence of SEQ ID NO:39; and (ii) a VL2 having an amino acid sequence of SEQ ID NO:43; or
(i) a VH2 having an amino acid sequence of SEQ ID NO:47; and (ii) a VL2 having an amino acid sequence of SEQ ID NO:51; or
(i) a VH2 having an amino acid sequence of SEQ ID NO:55; and (ii) a VL2 having an amino acid sequence of SEQ ID NO:59; or
(i) a VH2 having an amino acid sequence of SEQ ID NO:63; and (ii) a VL2 having an amino acid sequence of SEQ ID NO:67; or
(i) a VH2 having an amino acid sequence of SEQ ID NO:86; and (ii) a VL2 having an amino acid sequence of SEQ ID NO:90; or
(i) a VH2 having an amino acid sequence of SEQ ID NO:94; and (ii) a VL2 having an amino acid sequence of SEQ ID NO:98; or
(i) a VH2 having an amino acid sequence of SEQ ID NO:102; and (ii) a VL2 having an amino acid sequence of SEQ ID NO:106; or
(i) a VH2 having an amino acid sequence of SEQ ID NO:110; and (ii) a VL2 having an amino acid sequence of SEQ ID NO:114; or
(i) a VH2 having an amino acid sequence of SEQ ID NO:118; and (ii) a VL2 having an amino acid sequence of SEQ ID NO:122; or
(i) a VH2 having an amino acid sequence of SEQ ID NO:126; and (ii) a VL2 having an amino acid sequence of SEQ ID NO:130; or
(i) a VH2 having an amino acid sequence of SEQ ID NO:134; and (ii) a VL2 having an amino acid sequence of SEQ ID NO:138; or
(i) a VH2 having an amino acid sequence of SEQ ID NO:142; and (ii) a VL2 having an amino acid sequence of SEQ ID NO: 146; or
(i) a VH2 having an amino acid sequence of SEQ ID NO:150; and (ii) a VL2 having an amino acid sequence of SEQ ID NO: 154; or
(i) a VH2 having an amino acid sequence of SEQ ID NO:158; and (ii) a VL2 having an amino acid sequence of SEQ ID NO: 162; or
(i) a VH2 having an amino acid sequence of SEQ ID NO:166; and (ii) a VL2 having an amino acid sequence of SEQ ID NO:170; or
(i) a VH2 having an amino acid sequence of SEQ ID NO:174; and (ii) a VL2 having an amino acid sequence of SEQ ID NO:178; or
(i) a VH2 having an amino acid sequence of SEQ ID NO:182; and (ii) a VL2 having an amino acid sequence of SEQ ID NO:186; or
(i) a VH2 having an amino acid sequence of SEQ ID NO: 190; and (ii) a VL2 having an amino acid sequence of SEQ ID NO: 194; or
(i) a VH2 having an amino acid sequence of SEQ ID NO:198; and (ii) a VL2 having an amino acid sequence of SEQ ID NO:202; or
(i) a VH2 having an amino acid sequence of SEQ ID NO:380; and (ii) a VL2 having an amino acid sequence of SEQ ID NO:384; or
(i) a VH2 having an amino acid sequence of SEQ ID NO:388; and (ii) a VL2 having an amino acid sequence of SEQ ID NO:392; or
(i) a VH2 having an amino acid sequence of SEQ ID NO:396; and (ii) a VL2 having an amino acid sequence of SEQ ID NO:400; or
(i) a VH2 having an amino acid sequence of SEQ ID NO:404; and (ii) a VL2 having an amino acid sequence of SEQ ID NO:408; or
(i) a VH2 having an amino acid sequence of SEQ ID NO:412; and (ii) a VL2 having an amino acid sequence of SEQ ID NO:416; or
(i) a VH2 having an amino acid sequence of SEQ ID NO:420; and (ii) a VL2 having an amino acid sequence of SEQ ID NO:424.

In some embodiments, VH1 comprises a vhCDR1, a vhCDR2, and a vhCDR3 having an amino acid sequence of a vhCDR1, a vhCDR2, and a vhCDR3, respectively, of a VH having an amino acid sequence of SEQ ID NO:214; VL1 comprises a vlCDR1, a vICDR2, and a vlCDR3 having an amino acid sequence of a vlCDR1, a vlCDR2, and a vICDR3, respectively, of a VL having an amino acid sequence of SEQ ID NO:218; VH2 comprises a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and VL2 comprises a vlCDR1, a vlCDR2, and a vlCDR3 having an amino acid sequence of a vlCDR1, a vlCDR2, and a vlCDR3, respectively, of a VL having an amino acid sequence of SEQ ID NO:43.

In exemplary embodiments, VH1 comprises a vhCDR1 having an amino acid sequence of SEQ ID NO:215; a vhCDR2 having an amino acid sequence of SEQ ID NO:216; and a vhCDR3 having an amino acid sequence of SEQ ID NO:217; VL1 comprises a vICDR1 having an amino acid sequence of SEQ ID NO:219, a vlCDR2 having an amino acid sequence of SEQ ID NO: 220, and a vlCDR3 having an amino acid sequence of SEQ ID NO:221, VH2 comprises a vhCDR 1 having an amino acid sequence of SEQ ID NO:40; a vhCDR2 having an amino acid sequence of SEQ ID NO:41; and a vhCDR3 having an amino acid sequence of SEQ ID NO:42; and VL2 comprises a vlCDR1 having an amino acid sequence of SEQ ID NO:44, a vlCDR2 having an amino acid sequence of SEQ ID NO:45, and a vlCDR3 having an amino acid sequence of SEQ ID NO:46.

In some embodiments, the first Fc domain and second Fc domain are each variant Fc domains. In exemplary embodiments, one of the first and second Fc domains comprises heterodimerization variant T366W, and the other of the first and second Fc domains comprises heterodimerization variants T366S/L368A/Y407V, wherein numbering is according to EU numbering. In some embodiments, the first Fc domain comprises heterodimerization variant T366W, and the second Fc domain comprises heterodimerization variants T366S/L368A/Y407V.

In certain embodiments, the first and second Fc domains each comprise one or more ablation variants. In some embodiments, one or more ablation variants comprise L234A/L235A/D265S, wherein numbering is according to EU numbering.

In some embodiments, the first or second Fc domain comprises purification variants H435R/Y436F, wherein numbering is according to EU numbering.

In exemplary embodiments, the second Fc domain comprises purification variants H435R/Y436F.

In exemplary embodiments, the first Fc domain comprises amino acid substitutions L234A/L235A/D265S/LT366W, and the second Fc domain comprises amino acid substitutions L234A/L235A/D265S/T366S/L368A/Y407V/H435R/Y436F, wherein numbering is according to EU numbering. In some embodiments, the first and second Fc domains each further comprise amino acid substitutions M252Y/S254T/T256E, wherein numbering is according to EU numbering.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some emboidments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises a pI variant. In some embodiments, the CH1-hinge-CH2-CH3 of the second monomer comprises pI variants N208D/Q295E/N384D/Q418E/N421D, wherein numbering is according to EU numbering.

In some emboidments, the CH1-hinge-CH2-CH3 of the second monomer comprises amino acid variants

E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, of the anti-CD28 x anti-PSMA antibody, the scFv linker is selected from GKPGSGKPGSGKPGSGKPGS (SEQ ID NO: 443), GGSEGKSSGSGSESKSTGGS (SEQ ID NO: 456), and GGGSGGSGGCPPCGGSGG (SEQ ID NO: 457).

In another aspect, provided herein are heterodimeric anti-CD28 x anti-PSMA antibodies in the 2+1 mAb-scFv format. These antibodies comprise: a) a first monomer; b) a second monomer; and c) a light chain. The first monomer comprises, from N-terminal to C-terminal, VH1-CH1-hinge-CH2-CH3-domain linker-scFv, wherein VH1 is a first variable heavy domain, and CH2-CH3 is a first Fc domain. The second monomer comprises, from N-terminal to C-terminal, a VH1-CH1-hinge-CH2-CH3, wherein VH1 is a first variable heavy domain and CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). Each of the VH1s and the VL1 together form a first antigen binding domain (ABD), and the VH2 and the VL2 form a second ABD. Further, one of the first and second ABDs bind human CD28 and the other of the first and second ABDs binds PSMA.

In some embodiments, the first ABDs bind human CD28 and the second ABD binds PSMA. In certain embodiments, the first ABDs bind PSMA and the second ABD binds human CD28.

In some embodiments, the scFv comprises, from N-terminal to C-terminal, VH2-scFv linker-VL2. In certain embodiments, the scFv comprises, from N-terminal to C-terminal, VL2-scFv linker-VH2.

In some embodiments, the first ABDs bind to human PSMA, and VH1 and VL1 are selected from the following:
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:206; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:210; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:214; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:218; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:222; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:226; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:230; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:234; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:238; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:242; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:246; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:250; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:254; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:258; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:262; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:266; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:270; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:274; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:278; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:282; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:286; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:290; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:294; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:298; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:302; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:306; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:310; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:314; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:318; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:322; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:326; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:330; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:334; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:338.

In some embodiments, VH1 and VL1 are selected from the following:
(i) a VH1 comprising a vhCDR1 having an amino acid sequence of SEQ ID NO:207; a vhCDR2 having an amino acid sequence of SEQ ID NO:208; and a vhCDR3 having an amino acid sequence of SEQ ID NO:209; and (ii) a VL1 comprising a vlCDR1 having an amino acid sequence of SEQ ID NO:211, a vlCDR2 having an amino acid sequence of SEQ ID NO: 212, and a vlCDR3 having an amino acid sequence of SEQ ID NO:213; or
(i) a VH1 comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL1 comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221;
(i) a VH1 comprising a vhCDR1 having an amino acid sequence of SEQ ID NO:223; a vhCDR2 having an amino acid sequence of SEQ ID NO:224; and a vhCDR3 having an amino acid sequence of SEQ ID NO:225; and (ii) a VL1 comprising a vlCDR1 having an amino acid sequence of SEQ ID NO:227, a vlCDR2 having an amino acid sequence of SEQ ID NO: 228, and a vlCDR3 having an amino acid sequence of SEQ ID NO:229; or
(i) a VH1 comprising a vhCDR1 having an amino acid sequence of SEQ ID NO:231; a vhCDR2 having an amino acid sequence of SEQ ID NO:232; and a vhCDR3 having an amino acid sequence of SEQ ID NO:233; and (ii) a VL1 comprising a vlCDR1 having an amino acid sequence of SEQ ID NO:235, a vlCDR2 having an amino acid sequence of SEQ ID NO: 236, and a vlCDR3 having an amino acid sequence of SEQ ID NO:237; or
(i) a VH1 comprising a vhCDR1 having an amino acid sequence of SEQ ID NO:239; a vhCDR2 having an amino acid sequence of SEQ ID NO:240; and a vhCDR3 having an amino acid sequence of SEQ ID NO:241; and (ii) a VL1 comprising a vlCDR1 having an amino acid sequence of SEQ ID NO:243, a vlCDR2 having an amino acid sequence of SEQ ID NO: 244, and a vlCDR3 having an amino acid sequence of SEQ ID NO:245; or
(i) a VH1 comprising a vhCDR1 having an amino acid sequence of SEQ ID NO:247; a vhCDR2 having an amino acid sequence of SEQ ID NO:248; and a vhCDR3 having an amino acid sequence of SEQ ID NO:249; and (ii) a VL1 comprising a vlCDR1 having an amino acid sequence of SEQ ID NO:251, a vlCDR2 having an amino acid sequence of SEQ ID NO: 252, and a vlCDR3 having an amino acid sequence of SEQ ID NO:253; or
(i) a VH1 comprising a vhCDR1 having an amino acid sequence of SEQ ID NO:255; a vhCDR2 having an amino acid sequence of SEQ ID NO:256; and a vhCDR3 having an amino acid sequence of SEQ ID NO:257; and (ii) a VL1 comprising a vlCDR1 having an amino acid sequence of SEQ ID NO:259, a vlCDR2 having an amino acid sequence of SEQ ID NO: 260, and a vlCDR3 having an amino acid sequence of SEQ ID NO:261; or
(i) a VH1 comprising a vhCDR1 having an amino acid sequence of SEQ ID NO:263; a vhCDR2 having an amino acid sequence of SEQ ID NO:264; and a vhCDR3 having an amino acid sequence of SEQ ID NO:265; and (ii) a VL1 comprising a vlCDR1 having an amino acid sequence of SEQ ID NO:267, a vlCDR2 having an amino acid sequence of SEQ ID NO: 268, and a vlCDR3 having an amino acid sequence of SEQ ID NO:269; or
(i) a VH1 comprising a vhCDR1 having an amino acid sequence of SEQ ID NO:271; a vhCDR2 having an amino acid sequence of SEQ ID NO:212; and a vhCDR3 having an amino acid sequence of SEQ ID NO:273; and (ii) a VL1 comprising a vlCDR1 having an amino acid sequence of SEQ ID NO:275, a vlCDR2 having an amino acid sequence of SEQ ID NO: 276, and a vlCDR3 having an amino acid sequence of SEQ ID NO:277; or
(i) a VH1 comprising a vhCDR1 having an amino acid sequence of SEQ ID NO:279; a vhCDR2 having an amino acid sequence of SEQ ID NO:280; and a vhCDR3 having an amino acid sequence of SEQ ID NO:281; and (ii) a VL1 comprising a vlCDR1 having an amino acid sequence of SEQ ID NO:283, a vlCDR2 having an amino acid sequence of SEQ ID NO: 284, and a vlCDR3 having an amino acid sequence of SEQ ID NO:285; or
(i) a VH1 comprising a vhCDR1 having an amino acid sequence of SEQ ID NO:287; a vhCDR2 having an amino acid sequence of SEQ ID NO:288; and a vhCDR3 having an amino acid sequence of SEQ ID NO:289; and (ii) a VL1 comprising a vlCDR1 having an amino acid sequence of SEQ ID NO:291, a vlCDR2 having an amino acid sequence of SEQ ID NO: 292, and a vlCDR3 having an amino acid sequence of SEQ ID NO:293; or
(i) a VH1 comprising a vhCDR1 having an amino acid sequence of SEQ ID NO:295; a vhCDR2 having an amino acid sequence of SEQ ID NO:296; and a vhCDR3 having an amino acid sequence of SEQ ID NO:297; and (ii) a VL1 comprising a vlCDR1 having an amino acid sequence of SEQ ID NO:299, a vlCDR2 having an amino acid sequence of SEQ ID NO: 300, and a vlCDR3 having an amino acid sequence of SEQ ID NO:301; or
(i) a VH1 comprising a vhCDR1 having an amino acid sequence of SEQ ID NO:303; a vhCDR2 having an amino acid sequence of SEQ ID NO:304; and a vhCDR3 having an amino acid sequence of SEQ ID NO:305; and (ii) a VL1 comprising a vlCDR1 having an amino acid sequence of SEQ ID NO:307, a vlCDR2 having an amino acid sequence of SEQ ID NO: 308, and a vlCDR3 having an amino acid sequence of SEQ ID NO:309; or
(i) a VH1 comprising a vhCDR1 having an amino acid sequence of SEQ ID NO:311; a vhCDR2 having an amino acid sequence of SEQ ID NO:312; and a vhCDR3 having an amino acid sequence of SEQ ID NO:313; and (ii) a VL1 comprising a vlCDR1 having an amino acid sequence of SEQ ID NO:315, a vlCDR2 having an amino acid sequence of SEQ ID NO: 316, and a vlCDR3 having an amino acid sequence of SEQ ID NO:317; or
(i) a VH1 comprising a vhCDR1 having an amino acid sequence of SEQ ID NO:319; a vhCDR2 having an amino acid sequence of SEQ ID NO:320; and a vhCDR3 having an amino acid sequence of SEQ ID NO:321; and (ii) a VL1 comprising a vlCDR1 having an amino acid sequence of SEQ ID NO:323, a vlCDR2 having an amino acid sequence of SEQ ID NO: 324, and a vlCDR3 having an amino acid sequence of SEQ ID NO:325; or
(i) a VH1 comprising a vhCDR1 having an amino acid sequence of SEQ ID NO:327; a vhCDR2 having an amino acid sequence of SEQ ID NO:328; and a vhCDR3 having an amino acid sequence of SEQ ID NO:329; and (ii) a VL1 comprising a vlCDR1 having an amino acid sequence of SEQ ID NO:331, a vlCDR2 having an amino acid sequence of SEQ ID NO: 332, and a vlCDR3 having an amino acid sequence of SEQ ID NO:333; or
(i) a VH1 comprising a vhCDR1 having an amino acid sequence of SEQ ID NO:335; a vhCDR2 having an amino acid sequence of SEQ ID NO:336; and a vhCDR3 having an amino acid sequence of SEQ ID NO:337; and (ii) a VL1 comprising a vlCDR1 having an amino acid sequence of SEQ ID NO:339, a vlCDR2 having an amino acid sequence of SEQ ID NO: 340, and a vlCDR3 having an amino acid sequence of SEQ ID NO:341.

In exemplary embodiments, VH1 and VL1 are selected from the following:
(i) a VH1 having an amino acid sequence of SEQ ID NO:206; and (ii) a VL1 having an amino acid sequence of SEQ ID NO:210; or
(i) a VH1 having an amino acid sequence of SEQ ID NO:214; and (ii) a VL1 having an amino acid sequence of SEQ ID NO:218; or
(i) a VH1 having an amino acid sequence of SEQ ID NO:222; and (ii) a VL1 having an amino acid sequence of SEQ ID NO:226; or
(i) a VH1 having an amino acid sequence of SEQ ID NO:230; and (ii) a VL1 having an amino acid sequence of SEQ ID NO:234; or
(i) a VH1 having an amino acid sequence of SEQ ID NO:238; and (ii) a VL1 having an amino acid sequence of SEQ ID NO:242; or
(i) a VH1 having an amino acid sequence of SEQ ID NO:246; and (ii) a VL1 having an amino acid sequence of SEQ ID NO:250; or
(i) a VH1 having an amino acid sequence of SEQ ID NO:254; and (ii) a VL1 having an amino acid sequence of SEQ ID NO:258; or
(i) a VH1 having an amino acid sequence of SEQ ID NO:262; and (ii) a VL1 having an amino acid sequence of SEQ ID NO:266; or
(i) a VH1 having an amino acid sequence of SEQ ID NO:270; and (ii) a VL1 having an amino acid sequence of SEQ ID NO:274; or
(i) a VH1 having an amino acid sequence of SEQ ID NO:278; and (ii) a VL1 having an amino acid sequence of SEQ ID NO:282; or
(i) a VH1 having an amino acid sequence of SEQ ID NO:286; and (ii) a VL1 having an amino acid sequence of SEQ ID NO:290; or
(i) a VH1 having an amino acid sequence of SEQ ID NO:294; and (ii) a VL1 having an amino acid sequence of SEQ ID NO:298; or
(i) a VH1 having an amino acid sequence of SEQ ID NO:302; and (ii) a VL1 having an amino acid sequence of SEQ ID NO:306; or
(i) a VH1 having an amino acid sequence of SEQ ID NO:310; and (ii) a VL1 having an amino acid sequence of SEQ ID NO:314; or
(i) a VH1 having an amino acid sequence of SEQ ID NO:318; and (ii) a VL1 having an amino acid sequence of SEQ ID NO:322; or
(i) a VH1 having an amino acid sequence of SEQ ID NO:326; and (ii) a VL1 having an amino acid sequence of SEQ ID NO:330; or
(i) a VH1 having an amino acid sequence of SEQ ID NO:334; and (ii) a VL1 having an amino acid sequence of SEQ ID NO:338.

In some embodiments, the second ABD binds to human CD28, and VH2 and VL2 are selected from the following:
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:13; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:31; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:35; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:43; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:47; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:51; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:55; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:59; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:67; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:90; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:94; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:98; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 102; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:106; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:110; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:114; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 118; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 122; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:126; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:130; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:134; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 138; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:142; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 146; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:150; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:158; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:162; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:166; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 170; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:174; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:178; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 182; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 186; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 190; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 194; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 198; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:202; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:380; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:384; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:396; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:400; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:404; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:408; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:412; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:416; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:420; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:424.

In certain embodiments, VH2 and VL2 are selected from the following:
(i) a VH2 comprising a vhCDR1 having an amino acid sequence of SEQ ID NO:10; a vhCDR2 having an amino acid sequence of SEQ ID NO:11; and a vhCDR3 having an amino acid sequence of SEQ ID NO: 12; and (ii) a VL2 comprising a vlCDR1 having an amino acid sequence of SEQ ID NO: 14, a vlCDR2 having an amino acid sequence of SEQ ID NO: 15, and a vlCDR3 having an amino acid sequence of SEQ ID NO:16; or
(i) a VH2 comprising a vhCDR1 having an amino acid sequence of SEQ ID NO:32; a vhCDR2 having an amino acid sequence of SEQ ID NO:33; and a vhCDR3 having an amino acid sequence of SEQ ID NO:34; and (ii) a VL2 comprising a vlCDR1 having an amino acid sequence of SEQ ID NO:36, a vlCDR2 having an amino acid sequence of SEQ ID NO: 37, and a vlCDR3 having an amino acid sequence of SEQ ID NO:38; or
i) a VH2 comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL2 comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; or
i) a VH2 comprising a vhCDR1 having an amino acid sequence of SEQ ID NO:48; a vhCDR2 having an amino acid sequence of SEQ ID NO:49; and a vhCDR3 having an amino acid sequence of SEQ ID NO:50; and (ii) a VL2 comprising a vlCDR1 having an amino acid sequence of SEQ ID NO:52, a vlCDR2 having an amino acid sequence of SEQ ID NO: 53, and a vlCDR3 having an amino acid sequence of SEQ ID NO:54; or
i) a VH2 comprising a vhCDR1 having an amino acid sequence of SEQ ID NO:56; a vhCDR2 having an amino acid sequence of SEQ ID NO:57; and a vhCDR3 having an amino acid sequence of SEQ ID NO:58; and (ii) a VL2 comprising a vlCDR1 having an amino acid sequence of SEQ ID NO:60, a vlCDR2 having an amino acid sequence of SEQ ID NO:61, and a vlCDR3 having an amino acid sequence of SEQ ID NO:62; or
i) a VH2 comprising a vhCDR1 having an amino acid sequence of SEQ ID NO:64; a vhCDR2 having an amino acid sequence of SEQ ID NO:65; and a vhCDR3 having an amino acid sequence of SEQ ID NO:66; and (ii) a VL2 comprising a vlCDR1 having an amino acid sequence of SEQ ID NO:68, a vlCDR2 having an amino acid sequence of SEQ ID NO:69, and a vlCDR3 having an amino acid sequence of SEQ ID NO:70; or
i) a VH2 comprising a vhCDR1 having an amino acid sequence of SEQ ID NO:87; a vhCDR2 having an amino acid sequence of SEQ ID NO:88; and a vhCDR3 having an amino acid sequence of SEQ ID NO:89; and (ii) a VL2 comprising a vlCDR1 having an amino acid sequence of SEQ ID NO:91, a vlCDR2 having an amino acid sequence of SEQ ID NO:92, and a vlCDR3 having an amino acid sequence of SEQ ID NO:93; or
i) a VH2 comprising a vhCDR1 having an amino acid sequence of SEQ ID NO:95; a vhCDR2 having an amino acid sequence of SEQ ID NO:96; and a vhCDR3 having an amino acid sequence of SEQ ID NO:97; and (ii) a VL2 comprising a vlCDR1 having an amino acid sequence of SEQ ID NO:99, a vlCDR2 having an amino acid sequence of SEQ ID NO:100, and a vlCDR3 having an amino acid sequence of SEQ ID NO: 101; or
i) a VH2 comprising a vhCDR1 having an amino acid sequence of SEQ ID NO:103; a vhCDR2 having an amino acid sequence of SEQ ID NO:104; and a vhCDR3 having an amino acid sequence of SEQ ID NO: 105; and (ii) a VL2 comprising a vlCDR1 having an amino acid sequence of SEQ ID NO: 107, a vlCDR2 having an amino acid sequence of SEQ ID NO: 108, and a vlCDR3 having an amino acid sequence of SEQ ID NO:109; or
i) a VH2 comprising a vhCDR1 having an amino acid sequence of SEQ ID NO:111; a vhCDR2 having an amino acid sequence of SEQ ID NO:112; and a vhCDR3 having an amino acid sequence of SEQ ID NO:113; and (ii) a VL2 comprising a vlCDR1 having an amino acid sequence of SEQ ID NO:115, a vlCDR2 having an amino acid sequence of SEQ ID NO: 116, and a vlCDR3 having an amino acid sequence of SEQ ID NO:117; or
i) a VH2 comprising a vhCDR1 having an amino acid sequence of SEQ ID NO:119; a vhCDR2 having an amino acid sequence of SEQ ID NO:120; and a vhCDR3 having an amino acid sequence of SEQ ID NO: 121; and (ii) a VL2 comprising a vlCDR1 having an amino acid sequence of SEQ ID NO: 123, a vlCDR2 having an amino acid sequence of SEQ ID NO:124, and a vlCDR3 having an amino acid sequence of SEQ ID NO:125; or
i) a VH2 comprising a vhCDR1 having an amino acid sequence of SEQ ID NO:127; a vhCDR2 having an amino acid sequence of SEQ ID NO: 128; and a vhCDR3 having an amino acid sequence of SEQ ID NO: 129; and (ii) a VL2 comprising a vlCDR1 having an amino acid sequence of SEQ ID NO: 131, a vlCDR2 having an amino acid sequence of SEQ ID NO: 132, and a vlCDR3 having an amino acid sequence of SEQ ID NO:133; or
i) a VH2 comprising a vhCDR1 having an amino acid sequence of SEQ ID NO:135; a vhCDR2 having an amino acid sequence of SEQ ID NO:136; and a vhCDR3 having an amino acid sequence of SEQ ID NO:137; and (ii) a VL2 comprising a vlCDR1 having an amino acid sequence of SEQ ID NO:139, a vlCDR2 having an amino acid sequence of SEQ ID NO: 140, and a vlCDR3 having an amino acid sequence of SEQ ID NO:141; or
i) a VH2 comprising a vhCDR1 having an amino acid sequence of SEQ ID NO:143; a vhCDR2 having an amino acid sequence of SEQ ID NO:144; and a vhCDR3 having an amino acid sequence of SEQ ID NO:115; and (ii) a VL2 comprising a vlCDR1 having an amino acid sequence of SEQ ID NO:147, a vlCDR2 having an amino acid sequence of SEQ ID NO: 149, and a vlCDR3 having an amino acid sequence of SEQ ID NO: 149; or
i) a VH2 comprising a vhCDR1 having an amino acid sequence of SEQ ID NO:151; a vhCDR2 having an amino acid sequence of SEQ ID NO:152; and a vhCDR3 having an amino acid sequence of SEQ ID NO: 153; and (ii) a VL2 comprising a vlCDR1 having an amino acid sequence of SEQ ID NO:155, a vlCDR2 having an amino acid sequence of SEQ ID NO:156, and a vlCDR3 having an amino acid sequence of SEQ ID NO:157; or
i) a VH2 comprising a vhCDR1 having an amino acid sequence of SEQ ID NO:159; a vhCDR2 having an amino acid sequence of SEQ ID NO:160; and a vhCDR3 having an amino acid sequence of SEQ ID NO:161; and (ii) a VL2 comprising a vlCDR1 having an amino acid sequence of SEQ ID NO:163, a vlCDR2 having an amino acid sequence of SEQ ID NO: 164, and a vlCDR3 having an amino acid sequence of SEQ ID NO:165; or
i) a VH2 comprising a vhCDR1 having an amino acid sequence of SEQ ID NO:167; a vhCDR2 having an amino acid sequence of SEQ ID NO:168; and a vhCDR3 having an amino acid sequence of SEQ ID NO: 169; and (ii) a VL2 comprising a vlCDR1 having an amino acid sequence of SEQ ID NO: 171, a vlCDR2 having an amino acid sequence of SEQ ID NO: 172, and a vlCDR3 having an amino acid sequence of SEQ ID NO:173; or
i) a VH2 comprising a vhCDR1 having an amino acid sequence of SEQ ID NO:175; a vhCDR2 having an amino acid sequence of SEQ ID NO:176; and a vhCDR3 having an amino acid sequence of SEQ ID NO: 177; and (ii) a VL2 comprising a vlCDR1 having an amino acid sequence of SEQ ID NO: 179, a vlCDR2 having an amino acid sequence of SEQ ID NO: 180, and a vlCDR3 having an amino acid sequence of SEQ ID NO:181; or
i) a VH2 comprising a vhCDR1 having an amino acid sequence of SEQ ID NO:183; a vhCDR2 having an amino acid sequence of SEQ ID NO:184; and a vhCDR3 having an amino acid sequence of SEQ ID NO:185; and (ii) a VL2 comprising a vlCDR1 having an amino acid sequence of SEQ ID NO:187, a vlCDR2 having an amino acid sequence of SEQ ID NO:188, and a vlCDR3 having an amino acid sequence of SEQ ID NO:189; or
i) a VH2 comprising a vhCDR1 having an amino acid sequence of SEQ ID NO:191; a vhCDR2 having an amino acid sequence of SEQ ID NO: 192; and a vhCDR3 having an amino acid sequence of SEQ ID NO:193; and (ii) a VL2 comprising a vlCDR1 having an amino acid sequence of SEQ ID NO:195, a vlCDR2 having an amino acid sequence of SEQ ID NO: 196, and a vlCDR3 having an amino acid sequence of SEQ ID NO: 197; or
i) a VH2 comprising a vhCDR1 having an amino acid sequence of SEQ ID NO:199; a vhCDR2 having an amino acid sequence of SEQ ID NO:200; and a vhCDR3 having an amino acid sequence of SEQ ID NO:201; and (ii) a VL2 comprising a vlCDR1 having an amino acid sequence of SEQ ID NO:203, a vlCDR2 having an amino acid sequence of SEQ ID NO:204, and a vlCDR3 having an amino acid sequence of SEQ ID NO:205; or
i) a VH2 comprising a vhCDR1 having an amino acid sequence of SEQ ID NO:381; a vhCDR2 having an amino acid sequence of SEQ ID NO:382; and a vhCDR3 having an amino acid sequence of SEQ ID NO:383; and (ii) a VL2 comprising a vlCDR1 having an amino acid sequence of SEQ ID NO:385, a vlCDR2 having an amino acid sequence of SEQ ID NO:386, and a vlCDR3 having an amino acid sequence of SEQ ID NO:387; or
i) a VH2 comprising a vhCDR1 having an amino acid sequence of SEQ ID NO:389; a vhCDR2 having an amino acid sequence of SEQ ID NO:390; and a vhCDR3 having an amino acid sequence of SEQ ID NO:391; and (ii) a VL2 comprising a vlCDR1 having an amino acid sequence of SEQ ID NO:393, a vlCDR2 having an amino acid sequence of SEQ ID NO:394, and a vlCDR3 having an amino acid sequence of SEQ ID NO:395; or
i) a VH2 comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL2 comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403; or
i) a VH2 comprising a vhCDR1 having an amino acid sequence of SEQ ID NO:405; a vhCDR2 having an amino acid sequence of SEQ ID NO:406; and a vhCDR3 having an amino acid sequence of SEQ ID NO:407; and (ii) a VL2 comprising a vlCDR1 having an amino acid sequence of SEQ ID NO:409, a vlCDR2 having an amino acid sequence of SEQ ID NO:410, and a vlCDR3 having an amino acid sequence of SEQ ID NO:411; or
i) a VH2 comprising a vhCDR1 having an amino acid sequence of SEQ ID NO:413; a vhCDR2 having an amino acid sequence of SEQ ID NO:414; and a vhCDR3 having an amino acid sequence of SEQ ID NO:415; and (ii) a VL2 comprising a vlCDR1 having an amino acid sequence of SEQ ID NO:417, a vlCDR2 having an amino acid sequence of SEQ ID NO:418, and a vlCDR3 having an amino acid sequence of SEQ ID NO:419; or
i) a VH2 comprising a vhCDR1 having an amino acid sequence of SEQ ID NO:421; a vhCDR2 having an amino acid sequence of SEQ ID NO:422; and a vhCDR3 having an amino acid sequence of SEQ ID NO:423; and (ii) a VL2 comprising a vlCDR1 having an amino acid sequence of SEQ ID NO:425, a vlCDR2 having an amino acid sequence of SEQ ID NO:426, and a vlCDR3 having an amino acid sequence of SEQ ID NO:427.

In exemplary embodiments, VH2 and VL2 are selected from the following:
(i) a VH2 having an amino acid sequence of SEQ ID NO:9; and (ii) a VL2 having an amino acid sequence of SEQ ID NO: 13; or
(i) a VH2 having an amino acid sequence of SEQ ID NO:31; and (ii) a VL2 having an amino acid sequence of SEQ ID NO:35; or
(i) a VH2 having an amino acid sequence of SEQ ID NO:39; and (ii) a VL2 having an amino acid sequence of SEQ ID NO:43; or
(i) a VH2 having an amino acid sequence of SEQ ID NO:47; and (ii) a VL2 having an amino acid sequence of SEQ ID NO:51; or
(i) a VH2 having an amino acid sequence of SEQ ID NO:55; and (ii) a VL2 having an amino acid sequence of SEQ ID NO:59; or
(i) a VH2 having an amino acid sequence of SEQ ID NO:63; and (ii) a VL2 having an amino acid sequence of SEQ ID NO:67; or
(i) a VH2 having an amino acid sequence of SEQ ID NO:86; and (ii) a VL2 having an amino acid sequence of SEQ ID NO:90; or
(i) a VH2 having an amino acid sequence of SEQ ID NO:94; and (ii) a VL2 having an amino acid sequence of SEQ ID NO:98; or
(i) a VH2 having an amino acid sequence of SEQ ID NO:102; and (ii) a VL2 having an amino acid sequence of SEQ ID NO: 106; or
(i) a VH2 having an amino acid sequence of SEQ ID NO:110; and (ii) a VL2 having an amino acid sequence of SEQ ID NO:114; or
(i) a VH2 having an amino acid sequence of SEQ ID NO:118; and (ii) a VL2 having an amino acid sequence of SEQ ID NO: 122; or
(i) a VH2 having an amino acid sequence of SEQ ID NO:126; and (ii) a VL2 having an amino acid sequence of SEQ ID NO:130; or
(i) a VH2 having an amino acid sequence of SEQ ID NO:134; and (ii) a VL2 having an amino acid sequence of SEQ ID NO:138; or
(i) a VH2 having an amino acid sequence of SEQ ID NO:142; and (ii) a VL2 having an amino acid sequence of SEQ ID NO: 146; or
(i) a VH2 having an amino acid sequence of SEQ ID NO:150; and (ii) a VL2 having an amino acid sequence of SEQ ID NO:154; or
(i) a VH2 having an amino acid sequence of SEQ ID NO:158; and (ii) a VL2 having an amino acid sequence of SEQ ID NO:162; or
(i) a VH2 having an amino acid sequence of SEQ ID NO:166; and (ii) a VL2 having an amino acid sequence of SEQ ID NO:170; or
(i) a VH2 having an amino acid sequence of SEQ ID NO:174; and (ii) a VL2 having an amino acid sequence of SEQ ID NO:178; or
(i) a VH2 having an amino acid sequence of SEQ ID NO:182; and (ii) a VL2 having an amino acid sequence of SEQ ID NO: 186; or
(i) a VH2 having an amino acid sequence of SEQ ID NO:190; and (ii) a VL2 having an amino acid sequence of SEQ ID NO: 194; or
(i) a VH2 having an amino acid sequence of SEQ ID NO:198; and (ii) a VL2 having an amino acid sequence of SEQ ID NO:202; or
(i) a VH2 having an amino acid sequence of SEQ ID NO:380; and (ii) a VL2 having an amino acid sequence of SEQ ID NO:384; or
(i) a VH2 having an amino acid sequence of SEQ ID NO:388; and (ii) a VL2 having an amino acid sequence of SEQ ID NO:392; or
(i) a VH2 having an amino acid sequence of SEQ ID NO:396; and (ii) a VL2 having an amino acid sequence of SEQ ID NO:400; or
(i) a VH2 having an amino acid sequence of SEQ ID NO:404; and (ii) a VL2 having an amino acid sequence of SEQ ID NO:408; or
(i) a VH2 having an amino acid sequence of SEQ ID NO:412; and (ii) a VL2 having an amino acid sequence of SEQ ID NO:416; or
(i) a VH2 having an amino acid sequence of SEQ ID NO:420; and (ii) a VL2 having an amino acid sequence of SEQ ID NO:424.

In some embodiments, VH1 comprises a vhCDR1, a vhCDR2, and a vhCDR3 having an amino acid sequence of a vhCDR1, a vhCDR2, and a vhCDR3, respectively, of a VH having an amino acid sequence of SEQ ID NO:214; VL1 comprises a vlCDR1, a vICDR2, and a vlCDR3 having an amino acid sequence of a vlCDR1, a vlCDR2, and a vlCDR3, respectively, of a VL having an amino acid sequence of SEQ ID NO:218; VH2 comprises a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and VL2 comprises a vlCDR1, a vlCDR2, and a vlCDR3 having an amino acid sequence of a vlCDR1, a vlCDR2, and a vlCDR3, respectively, of a VL having an amino acid sequence of SEQ ID NO:43.

In exemplary embodiments, VH1 comprises a vhCDR1 having an amino acid sequence of SEQ ID NO:215; a vhCDR2 having an amino acid sequence of SEQ ID NO:216; and a vhCDR3 having an amino acid sequence of SEQ ID NO:217; VL1 comprises a vlCDR1 having an amino acid sequence of SEQ ID NO:219, a vlCDR2 having an amino acid sequence of SEQ ID NO: 220, and a vlCDR3 having an amino acid sequence of SEQ ID NO:221, VH2 comprises a vhCDR 1 having an amino acid sequence of SEQ ID NO:40; a vhCDR2 having an amino acid sequence of SEQ ID NO:41; and a vhCDR3 having an amino acid sequence of SEQ ID NO:42; and VL2 comprises a vlCDR1 having an amino acid sequence of SEQ ID NO:44, a vlCDR2 having an amino acid sequence of SEQ ID NO:45, and a vlCDR3 having an amino acid sequence of SEQ ID NO:46.

In some embodiments, the first Fc domain and second Fc domain are each variant Fc domains. In exemplary embodiments, one of the first and second Fc domains comprises heterodimerization variant T366W, and the other of the first and second Fc domains comprises heterodimerization variants T366S/L368A/Y407V, wherein numbering is according to EU numbering. In some embodiments, the first Fc domain comprises heterodimerization variant T366W, and the second Fc domain comprises heterodimerization variants T366S/L368A/Y407V.

In certain embodiments, the first and second Fc domains each comprise one or more ablation variants. In some embodiments, one or more ablation variants comprise L234A/L235A/D265S, wherein numbering is according to EU numbering.

In some embodiments, the first or second Fc domain comprises purification variants H435R/Y436F, wherein numbering is according to EU numbering.

In exemplary embodiments, the second Fc domain comprises purification variants H435R/Y436F.

In exemplary embodiments, the first Fc domain comprises amino acid substitutions L234A/L235A/D265S/LT366W, and the second Fc domain comprises amino acid substitutions L234A/L235A/D265S/T366S/L368A/Y407V/H435R/Y436F, wherein numbering is according to EU numbering. In some embodiments, the first and second Fc domains each further comprise amino acid substitutions M252Y/S254T/T256E, wherein numbering is according to EU numbering.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some emboidments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises a pI variant. In some embodiments, the CH1-hinge-CH2-CH3 of the second monomer comprises pI variants N208D/Q295E/N384D/Q418E/N421D, wherein numbering is according to EU numbering.

In some emboidments, the CH1-hinge-CH2-CH3 of the second monomer comprises amino acid variants

E233P/L234V/L235A/G236del/S267K/L368D/K370S/N208D/Q295E/N384D/Q418E/N421 D, the first Fc domain comprises amino acid variants

E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, of the anti-CD28 x anti-PSMA antibody, the scFv linker is selected from GKPGSGKPGSGKPGSGKPGS (SEQ ID NO: 443), GGSEGKSSGSGSESKSTGGS (SEQ ID NO: 456), and GGGSGGSGGCPPCGGSGG (SEQ ID NO: 457).

In another aspect, provided herein are bispecific antibodies that include a first antigen binding domain that binds to PSMA, and a second antigen binding domain that binds to CD28.

The first antigen binding domain (i.e., the PSMA binding domain) comprises:
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:206; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:210; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:214; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:218; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:222; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:226; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:230; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:234; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:238; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:242; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:246; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:250; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:254; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:258; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:262; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:266; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:270; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:274; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:278; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:282; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:286; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:290; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:294; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:298; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:302; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:306; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:310; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:314; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:318; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:322; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:326; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:330; or
(i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:334; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:338.

The second antigen binding domain (i.e., the CD28 binding domain) comprises:

i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 13; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:31; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:35; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:43; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:47; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:51; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:55; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:59; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:67; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:90; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:94; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:98; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:102; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 106; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 110; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:114; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 118; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 122; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 126; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 130; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 134; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:138; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 142; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:146; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 150; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:158; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:162; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 166; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:170; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 174; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 178; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:182; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 186; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 190; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:194; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 198; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:202; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:380; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:384; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:396; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:400; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:404; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:408; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:412; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:416; or
(i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:420; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:424.

In some embodiments, (i) the vhCDR1, vhCDR2, vhCDR3, vlCDR1, vlCDR2, and vlCDR3 amino acid sequences are according to the Kabat numbering system; (ii) the vhCDR1, vhCDR2, vhCDR3, vlCDR1, vlCDR2, and vlCDR3 amino acid sequences are according to the Chothia numbering system; (iii) the vhCDR1, vhCDR2, vhCDR3, VICDR1, vICDR2, and vlCDR3 amino acid sequences are according to the AbM numbering system;
(iv) the vhCDR1, vhCDR2, vhCDR3, vlCDR1, vlCDR2, and vlCDR3 amino acid sequences are according to the Contact numbering system; (v) wherein the vhCDR1, vhCDR2, vhCDR3, vlCDR1, vlCDR2, and vlCDR3 amino acid sequences are according to the IMGT numbering system; (vi) wherein the vhCDR1, vhCDR2, vhCDR3, vlCDR1, vlCDR2, and vlCDR3 amino acid sequences are according to the Kabat+Chothia numbering system; and/or the vhCDR1, vhCDR2, vhCDR3, vlCDR1, vlCDR2, and vlCDR3 amino acid sequences are according to the Xencor numbering system.

In another aspect, provided herein are bispecific antibodies that include a first antigen binding domain that binds to PSMA, and a second antigen binding domain that binds to CD28.

The first antigen binding domain (i.e., the PSMA binding domain) comprises:
(i) a VH1 comprising a vhCDR1 having an amino acid sequence of SEQ ID NO:207; a vhCDR2 having an amino acid sequence of SEQ ID NO:208; and a vhCDR3 having an amino acid sequence of SEQ ID NO:209; and (ii) a VL1 comprising a vlCDR1 having an amino acid sequence of SEQ ID NO:211, a vlCDR2 having an amino acid sequence of SEQ ID NO: 212, and a vlCDR3 having an amino acid sequence of SEQ ID NO:213; or
(i) a VH1 comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL1 comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221;
(i) a VH1 comprising a vhCDR1 having an amino acid sequence of SEQ ID NO:223; a vhCDR2 having an amino acid sequence of SEQ ID NO:224; and a vhCDR3 having an amino acid sequence of SEQ ID NO:225; and (ii) a VL1 comprising a vlCDR1 having an amino acid sequence of SEQ ID NO:227, a vlCDR2 having an amino acid sequence of SEQ ID NO: 228, and a vlCDR3 having an amino acid sequence of SEQ ID NO:229; or
(i) a VH1 comprising a vhCDR1 having an amino acid sequence of SEQ ID NO:231; a vhCDR2 having an amino acid sequence of SEQ ID NO:232; and a vhCDR3 having an amino acid sequence of SEQ ID NO:233; and (ii) a VL1 comprising a vlCDR1 having an amino acid sequence of SEQ ID NO:235, a vlCDR2 having an amino acid sequence of SEQ ID NO: 236, and a vlCDR3 having an amino acid sequence of SEQ ID NO:237; or
(i) a VH1 comprising a vhCDR1 having an amino acid sequence of SEQ ID NO:239; a vhCDR2 having an amino acid sequence of SEQ ID NO:240; and a vhCDR3 having an amino acid sequence of SEQ ID NO:241; and (ii) a VL1 comprising a vlCDR1 having an amino acid sequence of SEQ ID NO:243, a vlCDR2 having an amino acid sequence of SEQ ID NO: 244, and a vlCDR3 having an amino acid sequence of SEQ ID NO:245; or
(i) a VH1 comprising a vhCDR1 having an amino acid sequence of SEQ ID NO:247; a vhCDR2 having an amino acid sequence of SEQ ID NO:248; and a vhCDR3 having an amino acid sequence of SEQ ID NO:249; and (ii) a VL1 comprising a vlCDR1 having an amino acid sequence of SEQ ID NO:251, a vlCDR2 having an amino acid sequence of SEQ ID NO: 252, and a vlCDR3 having an amino acid sequence of SEQ ID NO:253; or
(i) a VH1 comprising a vhCDR1 having an amino acid sequence of SEQ ID NO:255; a vhCDR2 having an amino acid sequence of SEQ ID NO:256; and a vhCDR3 having an amino acid sequence of SEQ ID NO:257; and (ii) a VL1 comprising a vlCDR1 having an amino acid sequence of SEQ ID NO:259, a vlCDR2 having an amino acid sequence of SEQ ID NO: 260, and a vlCDR3 having an amino acid sequence of SEQ ID NO:261; or
(i) a VH1 comprising a vhCDR1 having an amino acid sequence of SEQ ID NO:263; a vhCDR2 having an amino acid sequence of SEQ ID NO:264; and a vhCDR3 having an amino acid sequence of SEQ ID NO:265; and (ii) a VL1 comprising a vlCDR1 having an amino acid sequence of SEQ ID NO:267, a vlCDR2 having an amino acid sequence of SEQ ID NO: 268, and a vlCDR3 having an amino acid sequence of SEQ ID NO:269; or
(i) a VH1 comprising a vhCDR1 having an amino acid sequence of SEQ ID NO:271; a vhCDR2 having an amino acid sequence of SEQ ID NO:212; and a vhCDR3 having an amino acid sequence of SEQ ID NO:273; and (ii) a VL1 comprising a vlCDR1 having an amino acid sequence of SEQ ID NO:275, a vlCDR2 having an amino acid sequence of SEQ ID NO: 276, and a vlCDR3 having an amino acid sequence of SEQ ID NO:277; or
(i) a VH1 comprising a vhCDR1 having an amino acid sequence of SEQ ID NO:279; a vhCDR2 having an amino acid sequence of SEQ ID NO:280; and a vhCDR3 having an amino acid sequence of SEQ ID NO:281; and (ii) a VL1 comprising a vlCDR1 having an amino acid sequence of SEQ ID NO:283, a vlCDR2 having an amino acid sequence of SEQ ID NO: 284, and a vlCDR3 having an amino acid sequence of SEQ ID NO:285; or
(i) a VH1 comprising a vhCDR1 having an amino acid sequence of SEQ ID NO:287; a vhCDR2 having an amino acid sequence of SEQ ID NO:288; and a vhCDR3 having an amino acid sequence of SEQ ID NO:289; and (ii) a VL1 comprising a vlCDR1 having an amino acid sequence of SEQ ID NO:291, a vlCDR2 having an amino acid sequence of SEQ ID NO: 292, and a vlCDR3 having an amino acid sequence of SEQ ID NO:293; or
(i) a VH1 comprising a vhCDR1 having an amino acid sequence of SEQ ID NO:295; a vhCDR2 having an amino acid sequence of SEQ ID NO:296; and a vhCDR3 having an amino acid sequence of SEQ ID NO:297; and (ii) a VL1 comprising a vlCDR1 having an amino acid sequence of SEQ ID NO:299, a vlCDR2 having an amino acid sequence of SEQ ID NO: 300, and a vlCDR3 having an amino acid sequence of SEQ ID NO:301; or
(i) a VH1 comprising a vhCDR1 having an amino acid sequence of SEQ ID NO:303; a vhCDR2 having an amino acid sequence of SEQ ID NO:304; and a vhCDR3 having an amino acid sequence of SEQ ID NO:305; and (ii) a VL1 comprising a vlCDR1 having an amino acid sequence of SEQ ID NO:307, a vlCDR2 having an amino acid sequence of SEQ ID NO: 308, and a vlCDR3 having an amino acid sequence of SEQ ID NO:309; or
(i) a VH1 comprising a vhCDR1 having an amino acid sequence of SEQ ID NO:311; a vhCDR2 having an amino acid sequence of SEQ ID NO:312; and a vhCDR3 having an amino acid sequence of SEQ ID NO:313; and (ii) a VL1 comprising a vlCDR1 having an amino acid sequence of SEQ ID NO:315, a vlCDR2 having an amino acid sequence of SEQ ID NO: 316, and a vlCDR3 having an amino acid sequence of SEQ ID NO:317; or
(i) a VH1 comprising a vhCDR1 having an amino acid sequence of SEQ ID NO:319; a vhCDR2 having an amino acid sequence of SEQ ID NO:320; and a vhCDR3 having an amino acid sequence of SEQ ID NO:321; and (ii) a VL1 comprising a vlCDR1 having an amino acid sequence of SEQ ID NO:323, a vlCDR2 having an amino acid sequence of SEQ ID NO: 324, and a vlCDR3 having an amino acid sequence of SEQ ID NO:325; or
(i) a VH1 comprising a vhCDR1 having an amino acid sequence of SEQ ID NO:327; a vhCDR2 having an amino acid sequence of SEQ ID NO:328; and a vhCDR3 having an amino acid sequence of SEQ ID NO:329; and (ii) a VL1 comprising a vlCDR1 having an amino acid sequence of SEQ ID NO:331, a vlCDR2 having an amino acid sequence of SEQ ID NO: 332, and a vlCDR3 having an amino acid sequence of SEQ ID NO:333; or
(i) a VH1 comprising a vhCDR1 having an amino acid sequence of SEQ ID NO:335; a vhCDR2 having an amino acid sequence of SEQ ID NO:336; and a vhCDR3 having an amino acid sequence of SEQ ID NO:337; and (ii) a VL1 comprising a vlCDR1 having an amino acid sequence of SEQ ID NO:339, a vlCDR2 having an amino acid sequence of SEQ ID NO: 340, and a vlCDR3 having an amino acid sequence of SEQ ID NO:341.

The second antigen binding domain (i.e., the CD28 binding domain) comprises:
(i) a VH2 comprising a vhCDR1 having an amino acid sequence of SEQ ID NO:10; a vhCDR2 having an amino acid sequence of SEQ ID NO:11; and a vhCDR3 having an amino acid sequence of SEQ ID NO:12; and (ii) a VL2 comprising a vlCDR1 having an amino acid sequence of SEQ ID NO: 14, a vlCDR2 having an amino acid sequence of SEQ ID NO: 15, and a vlCDR3 having an amino acid sequence of SEQ ID NO:16; or
(i) a VH2 comprising a vhCDR1 having an amino acid sequence of SEQ ID NO:32; a vhCDR2 having an amino acid sequence of SEQ ID NO:33; and a vhCDR3 having an amino acid sequence of SEQ ID NO:34; and (ii) a VL2 comprising a vlCDR1 having an amino acid sequence of SEQ ID NO:36, a vlCDR2 having an amino acid sequence of SEQ ID NO: 37, and a vlCDR3 having an amino acid sequence of SEQ ID NO:38; or
i) a VH2 comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL2 comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; or
i) a VH2 comprising a vhCDR1 having an amino acid sequence of SEQ ID NO:48; a vhCDR2 having an amino acid sequence of SEQ ID NO:49; and a vhCDR3 having an amino acid sequence of SEQ ID NO:50; and (ii) a VL2 comprising a vlCDR1 having an amino acid sequence of SEQ ID NO:52, a vlCDR2 having an amino acid sequence of SEQ ID NO: 53, and a vlCDR3 having an amino acid sequence of SEQ ID NO:54; or
i) a VH2 comprising a vhCDR1 having an amino acid sequence of SEQ ID NO:56; a vhCDR2 having an amino acid sequence of SEQ ID NO:57; and a vhCDR3 having an amino acid sequence of SEQ ID NO:58; and (ii) a VL2 comprising a vlCDR1 having an amino acid sequence of SEQ ID NO:60, a vlCDR2 having an amino acid sequence of SEQ ID NO:61, and a vlCDR3 having an amino acid sequence of SEQ ID NO:62; or
i) a VH2 comprising a vhCDR1 having an amino acid sequence of SEQ ID NO:64; a vhCDR2 having an amino acid sequence of SEQ ID NO:65; and a vhCDR3 having an amino acid sequence of SEQ ID NO:66; and (ii) a VL2 comprising a vlCDR1 having an amino acid sequence of SEQ ID NO:68, a vlCDR2 having an amino acid sequence of SEQ ID NO:69, and a vlCDR3 having an amino acid sequence of SEQ ID NO:70; or
i) a VH2 comprising a vhCDR1 having an amino acid sequence of SEQ ID NO:87; a vhCDR2 having an amino acid sequence of SEQ ID NO:88; and a vhCDR3 having an amino acid sequence of SEQ ID NO:89; and (ii) a VL2 comprising a vlCDR1 having an amino acid sequence of SEQ ID NO:91, a vlCDR2 having an amino acid sequence of SEQ ID NO:92, and a vlCDR3 having an amino acid sequence of SEQ ID NO:93; or
i) a VH2 comprising a vhCDR1 having an amino acid sequence of SEQ ID NO:95; a vhCDR2 having an amino acid sequence of SEQ ID NO:96; and a vhCDR3 having an amino acid sequence of SEQ ID NO:97; and (ii) a VL2 comprising a vlCDR1 having an amino acid sequence of SEQ ID NO:99, a vlCDR2 having an amino acid sequence of SEQ ID NO: 100, and a vlCDR3 having an amino acid sequence of SEQ ID NO: 101; or
i) a VH2 comprising a vhCDR1 having an amino acid sequence of SEQ ID NO:103; a vhCDR2 having an amino acid sequence of SEQ ID NO:104; and a vhCDR3 having an amino acid sequence of SEQ ID NO:105; and (ii) a VL2 comprising a vlCDR1 having an amino acid sequence of SEQ ID NO: 107, a vlCDR2 having an amino acid sequence of SEQ ID NO:108, and a vlCDR3 having an amino acid sequence of SEQ ID NO:109; or
i) a VH2 comprising a vhCDR1 having an amino acid sequence of SEQ ID NO:111; a vhCDR2 having an amino acid sequence of SEQ ID NO:112; and a vhCDR3 having an amino acid sequence of SEQ ID NO:113; and (ii) a VL2 comprising a vlCDR1 having an amino acid sequence of SEQ ID NO:115, a vlCDR2 having an amino acid sequence of SEQ ID NO: 116, and a vlCDR3 having an amino acid sequence of SEQ ID NO:117; or
i) a VH2 comprising a vhCDR1 having an amino acid sequence of SEQ ID NO:119; a vhCDR2 having an amino acid sequence of SEQ ID NO: 120; and a vhCDR3 having an amino acid sequence of SEQ ID NO: 121; and (ii) a VL2 comprising a vlCDR1 having an amino acid sequence of SEQ ID NO:123, a vlCDR2 having an amino acid sequence of SEQ ID NO: 124, and a vlCDR3 having an amino acid sequence of SEQ ID NO:125; or
i) a VH2 comprising a vhCDR1 having an amino acid sequence of SEQ ID NO:127; a vhCDR2 having an amino acid sequence of SEQ ID NO:128; and a vhCDR3 having an amino acid sequence of SEQ ID NO: 129; and (ii) a VL2 comprising a vlCDR1 having an amino acid sequence of SEQ ID NO:131, a vlCDR2 having an amino acid sequence of SEQ ID NO:132, and a vlCDR3 having an amino acid sequence of SEQ ID NO:133; or
i) a VH2 comprising a vhCDR1 having an amino acid sequence of SEQ ID NO:135; a vhCDR2 having an amino acid sequence of SEQ ID NO:136; and a vhCDR3 having an amino acid sequence of SEQ ID NO: 137; and (ii) a VL2 comprising a vlCDR1 having an amino acid sequence of SEQ ID NO: 139, a vlCDR2 having an amino acid sequence of SEQ ID NO:140, and a vlCDR3 having an amino acid sequence of SEQ ID NO:141; or
i) a VH2 comprising a vhCDR1 having an amino acid sequence of SEQ ID NO:143; a vhCDR2 having an amino acid sequence of SEQ ID NO:144; and a vhCDR3 having an amino acid sequence of SEQ ID NO: 115; and (ii) a VL2 comprising a vlCDR1 having an amino acid sequence of SEQ ID NO:147, a vlCDR2 having an amino acid sequence of SEQ ID NO:149, and a vlCDR3 having an amino acid sequence of SEQ ID NO:149; or
i) a VH2 comprising a vhCDR1 having an amino acid sequence of SEQ ID NO:151; a vhCDR2 having an amino acid sequence of SEQ ID NO:152; and a vhCDR3 having an amino acid sequence of SEQ ID NO:153; and (ii) a VL2 comprising a vlCDR1 having an amino acid sequence of SEQ ID NO: 155, a vlCDR2 having an amino acid sequence of SEQ ID NO:156, and a vlCDR3 having an amino acid sequence of SEQ ID NO:157; or
i) a VH2 comprising a vhCDR1 having an amino acid sequence of SEQ ID NO:159; a vhCDR2 having an amino acid sequence of SEQ ID NO:160; and a vhCDR3 having an amino acid sequence of SEQ ID NO: 161; and (ii) a VL2 comprising a vlCDR1 having an amino acid sequence of SEQ ID NO:163, a vlCDR2 having an amino acid sequence of SEQ ID NO:164, and a vlCDR3 having an amino acid sequence of SEQ ID NO:165; or
i) a VH2 comprising a vhCDR1 having an amino acid sequence of SEQ ID NO:167; a vhCDR2 having an amino acid sequence of SEQ ID NO:168; and a vhCDR3 having an amino acid sequence of SEQ ID NO: 169; and (ii) a VL2 comprising a vlCDR1 having an amino acid sequence of SEQ ID NO: 171, a vlCDR2 having an amino acid sequence of SEQ ID NO: 172, and a vlCDR3 having an amino acid sequence of SEQ ID NO:173; or
i) a VH2 comprising a vhCDR1 having an amino acid sequence of SEQ ID NO:175; a vhCDR2 having an amino acid sequence of SEQ ID NO:176; and a vhCDR3 having an amino acid sequence of SEQ ID NO: 177; and (ii) a VL2 comprising a vlCDR1 having an amino acid sequence of SEQ ID NO: 179, a vlCDR2 having an amino acid sequence of SEQ ID NO:180, and a vlCDR3 having an amino acid sequence of SEQ ID NO:181; or
i) a VH2 comprising a vhCDR1 having an amino acid sequence of SEQ ID NO:183; a vhCDR2 having an amino acid sequence of SEQ ID NO:184; and a vhCDR3 having an amino acid sequence of SEQ ID NO:185; and (ii) a VL2 comprising a vlCDR1 having an amino acid sequence of SEQ ID NO:187, a vlCDR2 having an amino acid sequence of SEQ ID NO: 188, and a vlCDR3 having an amino acid sequence of SEQ ID NO:189; or
i) a VH2 comprising a vhCDR1 having an amino acid sequence of SEQ ID NO:191; a vhCDR2 having an amino acid sequence of SEQ ID NO: 192; and a vhCDR3 having an amino acid sequence of SEQ ID NO: 193; and (ii) a VL2 comprising a vlCDR1 having an amino acid sequence of SEQ ID NO: 195, a vlCDR2 having an amino acid sequence of SEQ ID NO: 196, and a vlCDR3 having an amino acid sequence of SEQ ID NO:197; or
i) a VH2 comprising a vhCDR1 having an amino acid sequence of SEQ ID NO:199; a vhCDR2 having an amino acid sequence of SEQ ID NO:200; and a vhCDR3 having an amino acid sequence of SEQ ID NO:201; and (ii) a VL2 comprising a vlCDR1 having an amino acid sequence of SEQ ID NO:203, a vlCDR2 having an amino acid sequence of SEQ ID NO:204, and a vlCDR3 having an amino acid sequence of SEQ ID NO:205; or
i) a VH2 comprising a vhCDR1 having an amino acid sequence of SEQ ID NO:381; a vhCDR2 having an amino acid sequence of SEQ ID NO:382; and a vhCDR3 having an amino acid sequence of SEQ ID NO:383; and (ii) a VL2 comprising a vlCDR1 having an amino acid sequence of SEQ ID NO:385, a vlCDR2 having an amino acid sequence of SEQ ID NO:386, and a vlCDR3 having an amino acid sequence of SEQ ID NO:387; or
i) a VH2 comprising a vhCDR1 having an amino acid sequence of SEQ ID NO:389; a vhCDR2 having an amino acid sequence of SEQ ID NO:390; and a vhCDR3 having an amino acid sequence of SEQ ID NO:391; and (ii) a VL2 comprising a vlCDR1 having an amino acid sequence of SEQ ID NO:393, a vlCDR2 having an amino acid sequence of SEQ ID NO:394, and a vlCDR3 having an amino acid sequence of SEQ ID NO:395; or
i) a VH comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403; or
i) a VH2 comprising a vhCDR1 having an amino acid sequence of SEQ ID NO:405; a vhCDR2 having an amino acid sequence of SEQ ID NO:406; and a vhCDR3 having an amino acid sequence of SEQ ID NO:407; and (ii) a VL2 comprising a vlCDR1 having an amino acid sequence of SEQ ID NO:409, a vlCDR2 having an amino acid sequence of SEQ ID NO:410, and a vlCDR3 having an amino acid sequence of SEQ ID NO:411; or
i) a VH2 comprising a vhCDR1 having an amino acid sequence of SEQ ID NO:413; a vhCDR2 having an amino acid sequence of SEQ ID NO:414; and a vhCDR3 having an amino acid sequence of SEQ ID NO:415; and (ii) a VL2 comprising a vlCDR1 having an amino acid sequence of SEQ ID NO:417, a vlCDR2 having an amino acid sequence of SEQ ID NO:418, and a vlCDR3 having an amino acid sequence of SEQ ID NO:419; or
i) a VH2 comprising a vhCDR1 having an amino acid sequence of SEQ ID NO:421; a vhCDR2 having an amino acid sequence of SEQ ID NO:422; and a vhCDR3 having an amino acid sequence of SEQ ID NO:423; and (ii) a VL2 comprising a vlCDR1 having an amino acid sequence of SEQ ID NO:425, a vlCDR2 having an amino acid sequence of SEQ ID NO:426, and a vlCDR3 having an amino acid sequence of SEQ ID NO:427.

In another aspect, provided herein are bispecific antibodies that include a first antigen binding domain that binds to PSMA, and a second antigen binding domain that binds to CD28.

The first antigen binding domain (i.e., the PSMA binding domain) comprises:
(i) a VH1 having an amino acid sequence that is at least about 95% identical to SEQ ID NO:206; and (ii) a VL1 having an amino acid sequence that is at least about 95% identical to SEQ ID NO:210; or
(i) a VH1 having an amino acid sequence that is at least about 95% identical to SEQ ID NO:214; and (ii) a VL1 having an amino acid sequence that is at least about 95% identical to SEQ ID NO:218; or
(i) a VH1 having an amino acid sequence that is at least about 95% identical to SEQ ID NO:222; and (ii) a VL1 having an amino acid sequence that is at least about 95% identical to SEQ ID NO:226; or
(i) a VH1 having an amino acid sequence that is at least about 95% identical to SEQ ID NO:230; and (ii) a VL1 having an amino acid sequence that is at least about 95% identical to SEQ ID NO:234; or
(i) a VH1 having an amino acid sequence that is at least about 95% identical to SEQ ID NO:238; and (ii) a VL1 having an amino acid sequence that is at least about 95% identical to SEQ ID NO:242; or
(i) a VH1 having an amino acid sequence that is at least about 95% identical to SEQ ID NO:246; and (ii) a VL1 having an amino acid sequence that is at least about 95% identical to SEQ ID NO:250; or
(i) a VH1 having an amino acid sequence that is at least about 95% identical to SEQ ID NO:254; and (ii) a VL1 having an amino acid sequence that is at least about 95% identical to SEQ ID NO:258; or
(i) a VH1 having an amino acid sequence that is at least about 95% identical to SEQ ID NO:262; and (ii) a VL1 having an amino acid sequence that is at least about 95% identical to SEQ ID NO:266; or
(i) a VH1 having an amino acid sequence that is at least about 95% identical to SEQ ID NO:270; and (ii) a VL1 having an amino acid sequence that is at least about 95% identical to SEQ ID NO:274; or
(i) a VH1 having an amino acid sequence that is at least about 95% identical to SEQ ID NO:278; and (ii) a VL1 having an amino acid sequence that is at least about 95% identical to SEQ ID NO:282; or
(i) a VH1 having an amino acid sequence that is at least about 95% identical to SEQ ID NO:286; and (ii) a VL1 having an amino acid sequence that is at least about 95% identical to SEQ ID NO:290; or
(i) a VH1 having an amino acid sequence that is at least about 95% identical to SEQ ID NO:294; and (ii) a VL1 having an amino acid sequence that is at least about 95% identical to SEQ ID NO:298; or
(i) a VH1 having an amino acid sequence that is at least about 95% identical to SEQ ID NO:302; and (ii) a VL1 having an amino acid sequence that is at least about 95% identical to SEQ ID NO:306; or
(i) a VH1 having an amino acid sequence that is at least about 95% identical to SEQ ID NO:310; and (ii) a VL1 having an amino acid sequence that is at least about 95% identical to SEQ ID NO:314; or
(i) a VH1 having an amino acid sequence that is at least about 95% identical to SEQ ID NO:318; and (ii) a VL1 having an amino acid sequence that is at least about 95% identical to SEQ ID NO:322; or
(i) a VH1 having an amino acid sequence that is at least about 95% identical to SEQ ID NO:326; and (ii) a VL1 having an amino acid sequence that is at least about 95% identical to SEQ ID NO:330; or
(i) a VH1 having an amino acid sequence that is at least about 95% identical to SEQ ID NO:334; and (ii) a VL1 having an amino acid sequence that is at least about 95% identical to SEQ ID NO:338.

The second antigen binding domain (i.e., the CD28 binding domain) comprises:
(i) a VH2 having an amino acid sequence that is at least about 95% identical to SEQ ID NO:9; and (ii) a VL2 having an amino acid sequence that is at least about 95% identical to SEQ ID NO:13; or
(i) a VH2 having an amino acid sequence that is at least about 95% identical to SEQ ID NO:31; and (ii) a VL2 having an amino acid sequence that is at least about 95% identical to SEQ ID NO:35; or
(i) a VH2 having an amino acid sequence that is at least about 95% identical to SEQ ID NO:39; and (ii) a VL2 having an amino acid sequence that is at least about 95% identical to SEQ ID NO:43; or
(i) a VH2 having an amino acid sequence that is at least about 95% identical to SEQ ID NO:47; and (ii) a VL2 having an amino acid sequence that is at least about 95% identical to SEQ ID NO:51; or
(i) a VH2 having an amino acid sequence that is at least about 95% identical to SEQ ID NO:55; and (ii) a VL2 having an amino acid sequence that is at least about 95% identical to SEQ ID NO:59; or
(i) a VH2 having an amino acid sequence that is at least about 95% identical to SEQ ID NO:63; and (ii) a VL2 having an amino acid sequence that is at least about 95% identical to SEQ ID NO:67; or
(i) a VH2 having an amino acid sequence that is at least about 95% identical to SEQ ID NO:86; and (ii) a VL2 having an amino acid sequence that is at least about 95% identical to SEQ ID NO:90; or
(i) a VH2 having an amino acid sequence that is at least about 95% identical to SEQ ID NO:94; and (ii) a VL2 having an amino acid sequence that is at least about 95% identical to SEQ ID NO:98; or
(i) a VH2 having an amino acid sequence that is at least about 95% identical to SEQ ID NO:102; and (ii) a VL2 having an amino acid sequence that is at least about 95% identical to SEQ ID NO:106; or
(i) a VH2 having an amino acid sequence that is at least about 95% identical to SEQ ID NO:110; and (ii) a VL2 having an amino acid sequence that is at least about 95% identical to SEQ ID NO:114; or
(i) a VH2 having an amino acid sequence that is at least about 95% identical to SEQ ID NO:118; and (ii) a VL2 having an amino acid sequence that is at least about 95% identical to SEQ ID NO:122; or
(i) a VH2 having an amino acid sequence that is at least about 95% identical to SEQ ID NO:126; and (ii) a VL2 having an amino acid sequence that is at least about 95% identical to SEQ ID NO:130; or
(i) a VH2 having an amino acid sequence that is at least about 95% identical to SEQ ID NO:134; and (ii) a VL2 having an amino acid sequence that is at least about 95% identical to SEQ ID NO:138; or
(i) a VH2 having an amino acid sequence that is at least about 95% identical to SEQ ID NO:142; and (ii) a VL2 having an amino acid sequence that is at least about 95% identical to SEQ ID NO:146; or
(i) a VH2 having an amino acid sequence that is at least about 95% identical to SEQ ID NO:150; and (ii) a VL2 having an amino acid sequence that is at least about 95% identical to SEQ ID NO:154; or
(i) a VH2 having an amino acid sequence that is at least about 95% identical to SEQ ID NO:158; and (ii) a VL2 having an amino acid sequence that is at least about 95% identical to SEQ ID NO:162; or
(i) a VH2 having an amino acid sequence that is at least about 95% identical to SEQ ID NO:166; and (ii) a VL2 having an amino acid sequence that is at least about 95% identical to SEQ ID NO:170; or
(i) a VH2 having an amino acid sequence that is at least about 95% identical to SEQ ID NO:174; and (ii) a VL2 having an amino acid sequence that is at least about 95% identical to SEQ ID NO:178; or
(i) a VH2 having an amino acid sequence that is at least about 95% identical to SEQ ID NO:182; and (ii) a VL2 having an amino acid sequence that is at least about 95% identical to SEQ ID NO:186; or
(i) a VH2 having an amino acid sequence that is at least about 95% identical to SEQ ID NO:190; and (ii) a VL2 having an amino acid sequence that is at least about 95% identical to SEQ ID NO:194; or
(i) a VH2 having an amino acid sequence that is at least about 95% identical to SEQ ID NO:198; and (ii) a VL2 having an amino acid sequence that is at least about 95% identical to SEQ ID NO:202; or
(i) a VH2 having an amino acid sequence that is at least about 95% identical to SEQ ID NO:380; and (ii) a VL2 having an amino acid sequence that is at least about 95% identical to SEQ ID NO:384; or
(i) a VH2 having an amino acid sequence that is at least about 95% identical to SEQ ID NO:388; and (ii) a VL2 having an amino acid sequence that is at least about 95% identical to SEQ ID NO:392; or
(i) a VH2 having an amino acid sequence that is at least about 95% identical to SEQ ID NO:396; and (ii) a VL2 having an amino acid sequence that is at least about 95% identical to SEQ ID NO:400; or
(i) a VH2 having an amino acid sequence that is at least about 95% identical to SEQ ID NO:404; and (ii) a VL2 having an amino acid sequence that is at least about 95% identical to SEQ ID NO:408; or
(i) a VH2 having an amino acid sequence that is at least about 95% identical to SEQ ID NO:412; and (ii) a VL2 having an amino acid sequence that is at least about 95% identical to SEQ ID NO:416; or
(i) a VH2 having an amino acid sequence that is at least about 95% identical to SEQ ID NO:420; and (ii) a VL2 having an amino acid sequence that is at least about 95% identical to SEQ ID NO:424.

In some embodiments of the bispecific antibodies, the antibody is a humanized antibody. In certain embodiments, the antibody is a human antibody.

In some embodiments of the bispecific antibodies, the antibody is an IgG antibody. In some embodiments, IgG antibody is an IgG1, IgG2, IgG3, or IgG4 antibody. In exemplary embodiments, the IgG antibody is an IgG1 antibody.

In some embodiments of the bispecific antibodies, the first antigen binding domain binds a PSMA antigen. In exemplary embodiments, the first antigen binding domain binds a PSMA epitope. In some emboidments, the first antigen binding domain specifically binds to PSMA.

In some embodiments of the bispecific antibodies, the vhCDR1, vhCDR2, vhCDR3, vlCDR1, vlCDR2 and vlCDR3 of the first antigen binding domain form a binding site for an antigen of the PSMA. In certain embodiments, the vhCDR1, vhCDR2, vhCDR3, vICDR1, vlCDR2 and vlCDR3 of the first antigen binding domain form a binding site for an epitope of the PSMA.

In some embodiments of the bispecific antibodies, the second antigen binding

domain binds a CD28 antigen. In exemplary embodiments, the second antigen binding domain binds a CD28 epitope. In some emboidments, the second antigen binding domain specifically binds to CD28.

In some embodiments of the bispecific antibodies, the vhCDR1, vhCDR2, vhCDR3, vlCDR1, vlCDR2 and vlCDR3 of the second antigen binding domain form a binding site for an antigen of the CD28. In exemplary embodiments, the vhCDR1, vhCDR2, vhCDR3, vlCDR1, vlCDR2 and vlCDR3 of the second antigen binding domain form a binding site for an epitope of the CD28.

In some embodiments of the bispecific antibodies, the PSMA is present on the surface of a cell. In certain embodiments, the cell is a prostate cell. In exemplary embodiments, the cell is a prostate cancer cell.

In some embodiments of the bispecific antibodies, the antibody comprises a first Fc domain and a second Fc domain. In exemplary embodiments, the first Fc domain and the second Fc domain are each variant Fc domains.

In some embodiments, one of the first and second Fc domains comprises heterodimerization variant T366W, and the other of the first and second Fc domains comprises heterodimerization variants T366S/L368A/Y407V, wherein numbering is according to EU numbering. In exemplary embodiments, the first Fc domain comprises heterodimerization variant T366W, and the second Fc domain comprises heterodimerization variants T366S/L368A/Y407V.

In some embodiments, the first and second Fc domains each comprise one or more ablation variants. In exemplary embodiments, the one or more ablation variants comprise L234A/L235A/D265S, wherein numbering is according to EU numbering. I

n some embodiments, the first or second Fc domain comprises purification variants H435R/Y436F, wherein numbering is according to EU numbering. In exemplary embodiments, the second Fc domain comprises purification variants H435R/Y436F.

In certain embodiments, the first Fc domain comprises amino acid substitutions L234A/L235A/D265S/LT366W, and the second Fc domain comprises amino acid substitutions L234A/L235A/D265S/T366S/L368A/Y407V/H435R/Y436F, wherein numbering is according to EU numbering. In exemplary embodiments, the first and second Fc domains each further comprise amino acid substitutions M252Y/S254T/T256E, wherein numbering is according to EU numbering.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In exemplary embodiments, the first and second Fc domains comprise heterodimerization skew variants S364K/E357Q: L368D/K370S, wherein numbering is according to EU numbering.

In some embodiments, the first and second Fc domains each comprise one or more ablation variants. In some embodiments, the one or more ablation variants comprise E233P/L234V/L235A/G236del/S267K, wherein numbering is according to EU numbering.

In some emboidments of the bispecific antibody, the bispecific antibody is in the 1+1 Fab-scFv-Fc format. These antibodies comprise: a) a first monomer; b) a second monomer; and c) a light chain. The first monomer comprises: i) a single chain variable fragment (scFv); and ii) the first Fc domain, wherein the scFv is covalently attached to the N-terminus of the first Fc domain using a domain linker. The second monomer comprises, from N-terminal to C-terminal, a VH1-CH1-hinge-CH2-CH3, wherein VH1 is the first variable heavy domain and CH2-CH3 is the second Fc domain. The light chain comprises, from N-terminal to C-terminal, VL1-CL, wherein VL1 is the first variable light domain and CL is a constant light domain. The scFv comprises the second VH domain (VH2), a scFv linker, and the second variable light domain (VL2). The VH1 and the VL1 together form the first antigen binding domain (ABD) and the VH2 and the VL2 together form the second ABD.

In some embodiments, one of the first or second monomer further comprises a pI variant. In some embodiments, the CH1-hinge-CH2-CH3 of the second monomer comprises pI variants N208D/Q295E/N384D/Q418E/N421D, wherein numbering is according to EU numbering.

In exemplary embodiments, the CH1-hinge-CH2-CH3 of the second monomer comprises amino acid variants

E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In exemplary embodiments of the bispecific 1+1 Fab-scFv-Fc format antibody, the scFv linker is selected from GKPGSGKPGSGKPGSGKPGS (SEQ ID NO: 443), GGSEGKSSGSGSESKSTGGS (SEQ ID NO: 456), and GGGSGGSGGCPPCGGSGG (SEQ ID NO: 457).

In another aspect, provided herein is a heterodimeric antibody comprising: a) a first monomer having an amino acid sequence of SEQ ID NO:351; b) a second monomer having an amino acid sequence of SEQ ID NO:352; and c) a light chain having an amino acid sequence of SEQ ID NO:353.

In yet another aspect, provided herein is a heterodimeric antibody comprising: a) a first monomer having an amino acid sequence of SEQ ID NO:342; b) a second monomer having an amino acid sequence of SEQ ID NO:343; and c) a light chain having an amino acid sequence of SEQ ID NO:344.

In one aspect, provided herein is a bispecific antibody comprising: a) a first antigen binding domain having: (i) a VH1 having an amino acid sequence of SEQ ID NO:214; and (ii) a VL1 having an amino acid sequence of SEQ ID NO:218; and b) a second antigen binding domain having: (i) a VH2 having an amino acid sequence of SEQ ID NO:39; and (ii) a VL2 having an amino acid sequence of SEQ ID NO:43.

In one aspect, provided herein is a bispecific antibody comprising: a) a first antigen binding domain having: (i) a VH1 comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL1 comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221; and b) a second antigen binding domain having: (i) a VH2 comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL2 comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46.

In another aspect, provided herein is a bispecific antibody comprising: a) a first antigen binding domain having: (i) a VH1 having an amino acid sequence of SEQ ID NO:214; and (ii) a VL1 having an amino acid sequence of SEQ ID NO:218; and b) a second antigen binding domain having: (i) a VH2 having an amino acid sequence of SEQ ID NO:396; and (ii) a VL2 having an amino acid sequence of SEQ ID NO:400.

In one aspect, provided herein is a bispecific antibody comprising: a) a first antigen binding domain having: (i) a VH1 comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL1 comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221; and b) a second antigen binding domain having: (i) a VH2 comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL2 comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403.

Also provided herein are nucleic acid compositions comprising nucleic acids encoding the antibodies described herein, expression vector compositions that include such nucleic acids, host cells for making the antibodies that comprise the expression vector compositions, and methods of making the antibodies.

In another aspect, provided herein is a method of treating prostate cancer in a patient in need thereof, comprising administering to the patient an anti-CD28 x anti-PSMA antibody described herein.

In yet another aspect, provided herein is a method of treating prostate cancer in a patient in need thereof, comprising administering to the patient: a) an anti-CD28 x anti-PSMA as described herein; and b) an anti-CD3 x anti-PSMA an anti-CD3 x anti-B7H3, an anti-CD3 x anti-hK2 or an anti-CD3 x anti-TMEFF2 antibody.

In one aspect, provided herein is a method of enhancing T cell proliferation in the presence of PSMA-expressing cells, comprising contacting the cells with an anti-CD28 x anti-PSMA as described herein. In some embodiments, the PSMA-expressing cells are prostate cells.

In another aspect, provided herein is a method of inhibiting the growth or proliferation of PSMA-expressing cells, comprising contacting the cells with with an anti-CD28 x anti-PSMA as described herein. In some embodiments, the PSMA-expressing cells are prostate cells.

### BRIEF DESCRIPTION OF THE DRAWINGS

FIGS. 1A-1B depict the sequences for human, mouse, and cynomolgus CD28. Such CD28 are useful for the development of cross-reactive CD28 antigen binding domains for ease of clinical development.

FIGS. 2A-2B depicts the sequences for human, mouse, and cynomolgus PSMA. Such PSMA are useful for the development of cross-reactive PSMA antigen binding domains for ease of clinical development.

FIGS. 3A-3F depict useful pairs of heterodimerization variant sets (including skew and pI variants). In FIG. 3F, there are variants for which there are no corresponding “monomer 2” variants. Such variants are pI variants that can be used alone on either monomer of a bispecific antibody (e.g., PSMA x CD28 bsAb), or included, for example, on the non-scFv side of a format that utilizes an scFv as a component and an appropriate charged scFv linker can be used on the second monomer that utilizes an scFv as the CD28 binding domain. Suitable charged linkers are shown in FIGS. 6A-6B.

FIG. 4 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 5 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIGS. 6A-6B depicts a number of charged scFv linkers that find use in increasing or decreasing the pI of the subject heterodimeric bispecific antibodies that utilize one or more scFv as a component, as described herein (e.g., PSMA x CD28 bsAbs). The (+H) positive linker finds particular use herein, particularly with anti-CD28 VL and VH sequences shown herein. A single prior art scFv linker with a single charge is referenced as “Whitlow,” from Whitlow et al., Protein Engineering 6(8): 989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIG. 7 depicts a number of exemplary domain linkers. In some embodiments, these linkers find use linking a single-chain Fv to an Fc chain. In some embodiments, these linkers may be combined in any orientation. For example, a GGGGS (SEQ ID NO: 458) linker may be combined with a “lower half hinge” linker at the N-terminus or at the C-terminus.

FIG. 8 shows a particularly useful bispecific antibody platforms for the PSMA x CD28 bsAbs of the invention. Although the platforms are described here in the context of the 1+1 Fab-scFv-Fc format, they can be adapted for use in other bispecific antibody formats.

FIG. 9 depicts various heterodimeric skew variant amino acid substitutions that can be used with the heterodimeric antibodies described herein.

FIGS. 10A-10E shows the sequences of several useful heterodimeric PSMA x CD28 bsAb backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants on both chains. Heterodimeric Fc backbone 14 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants and M252Y/S254T/T256E half-life extension variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 16 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 17 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

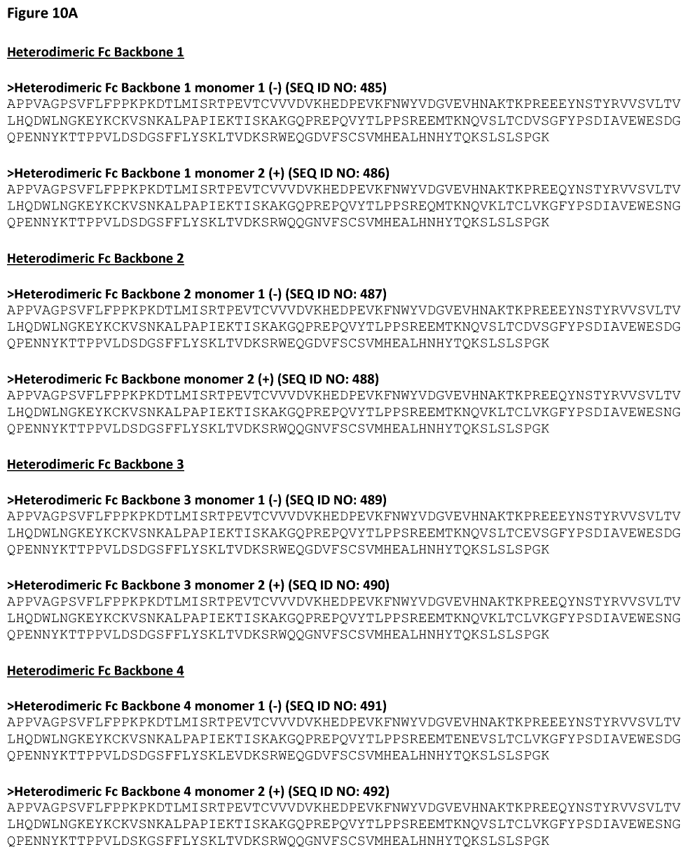

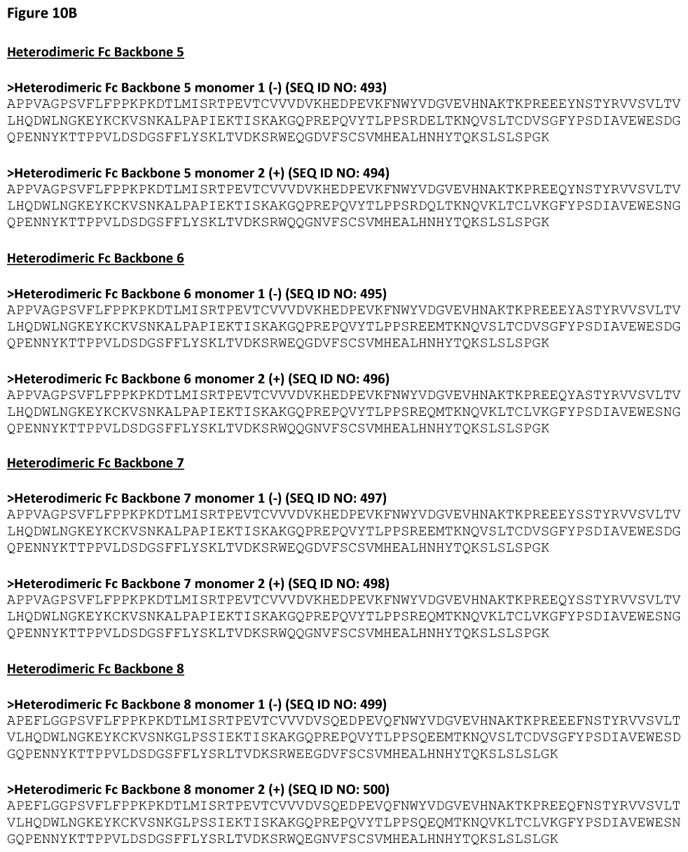

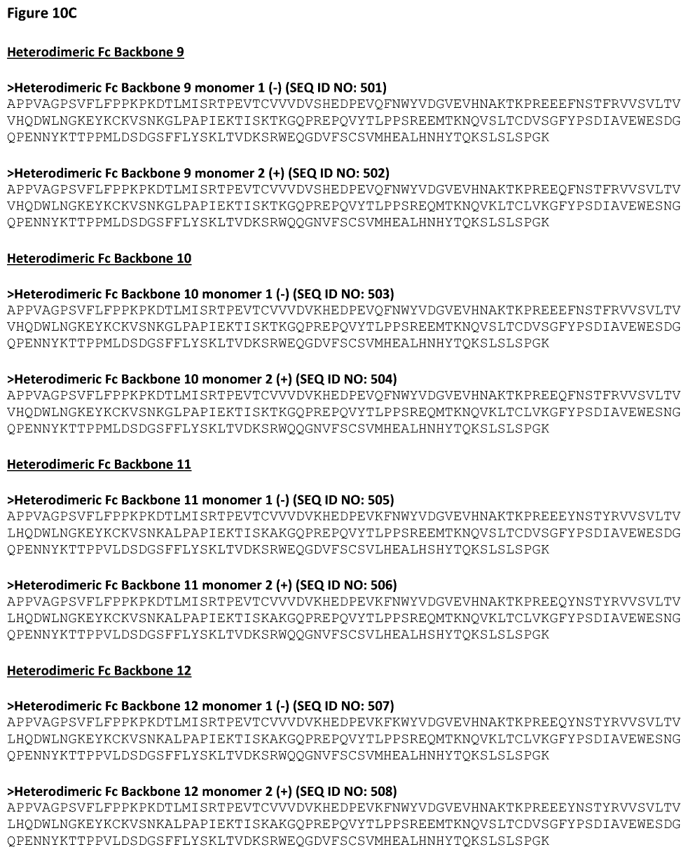

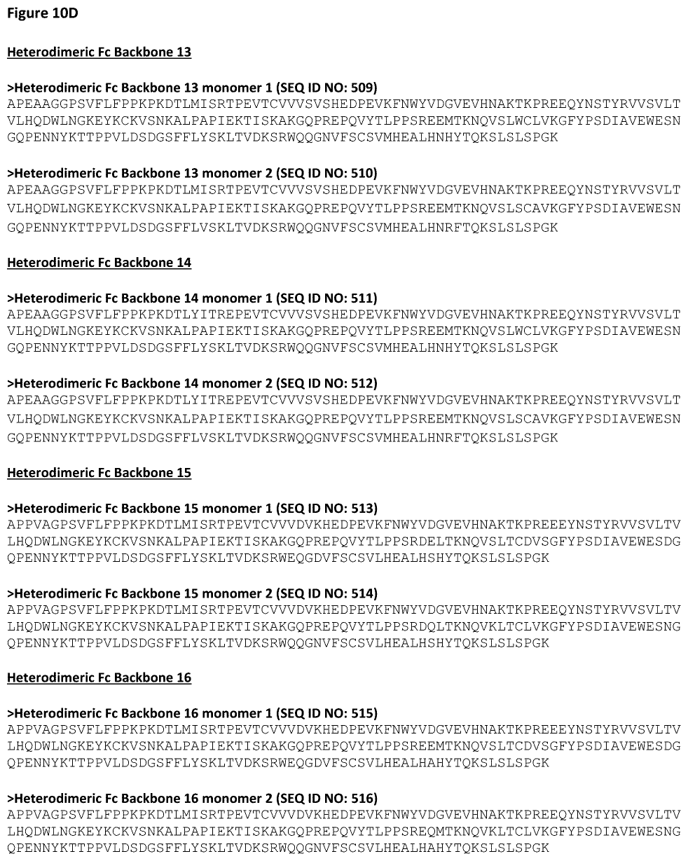

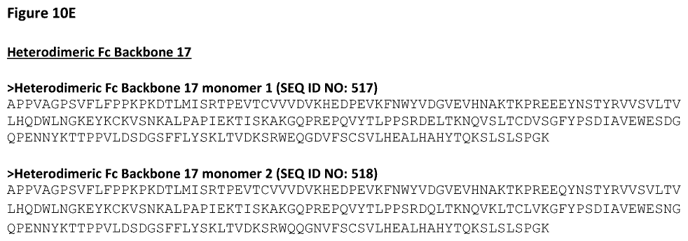

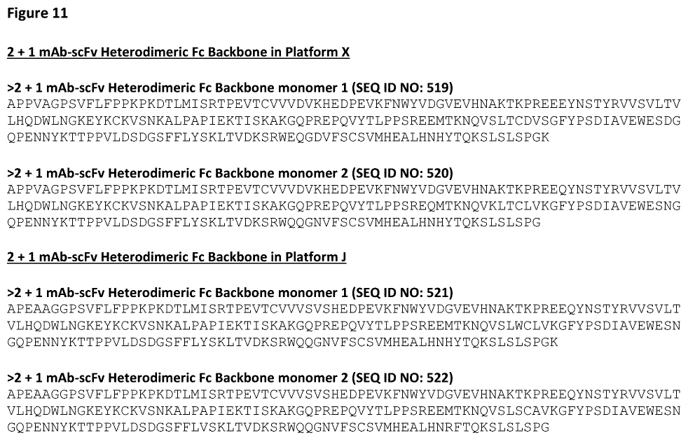

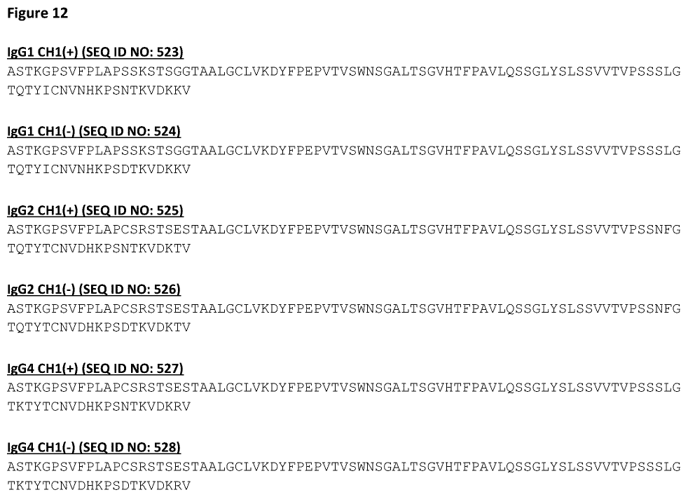

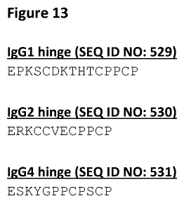

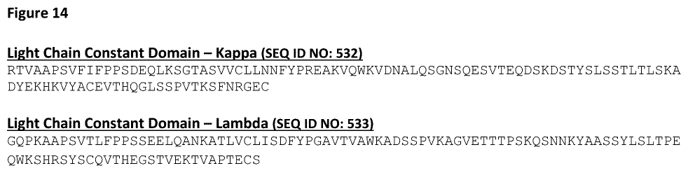

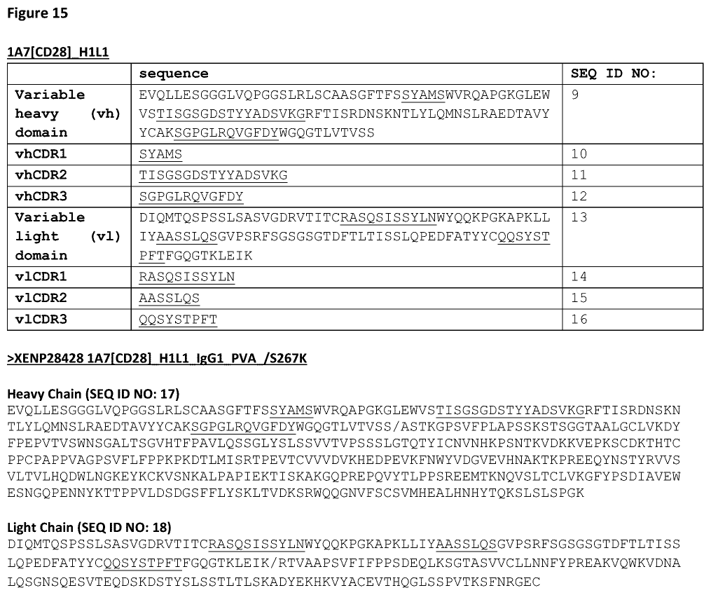

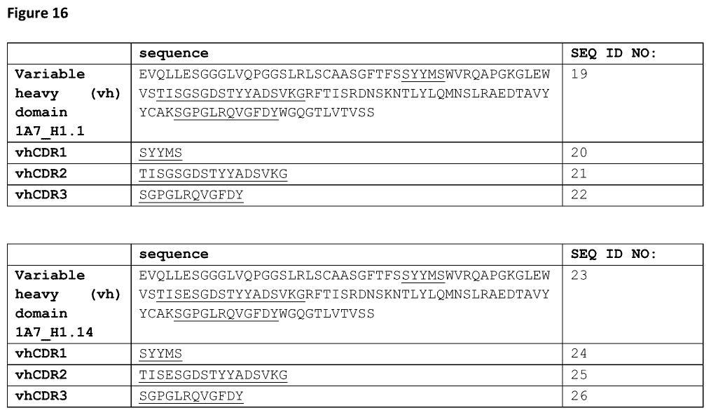

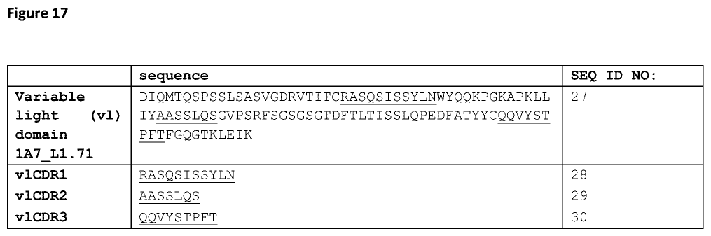

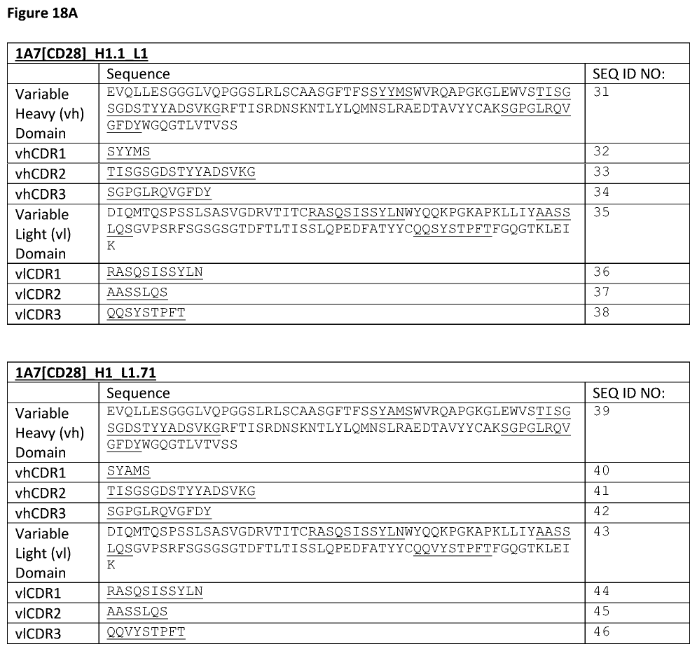

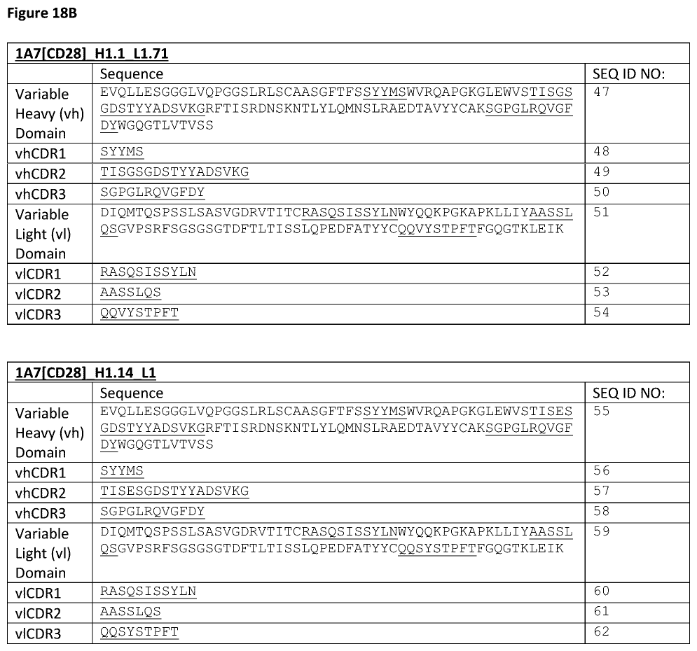

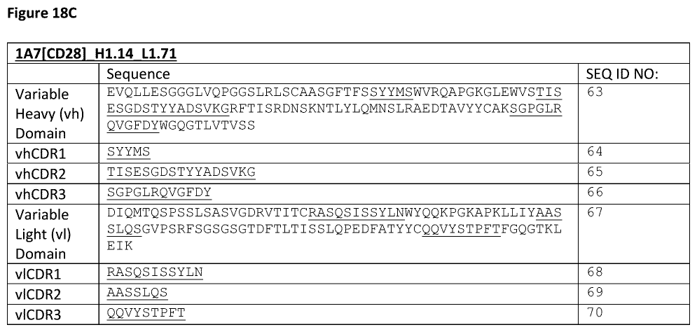

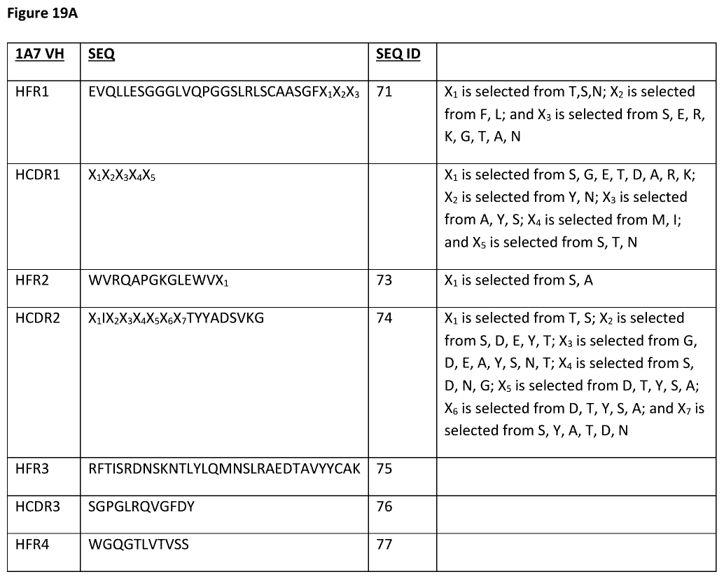

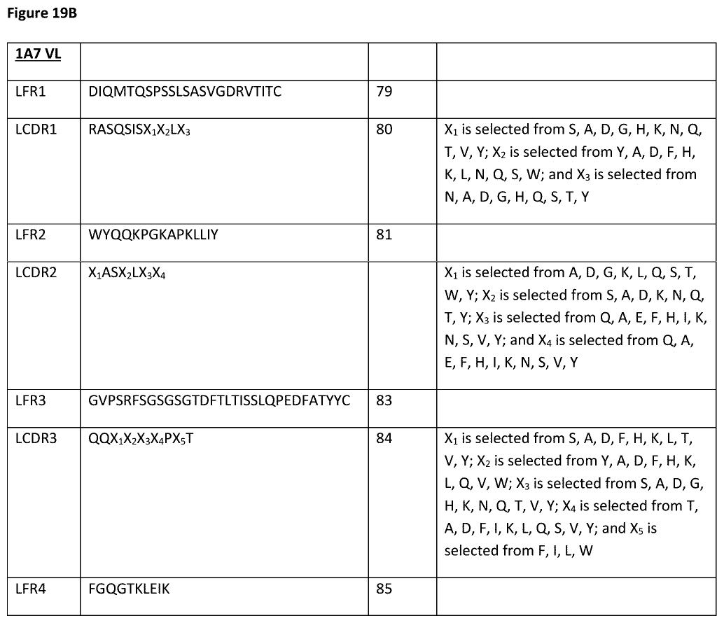

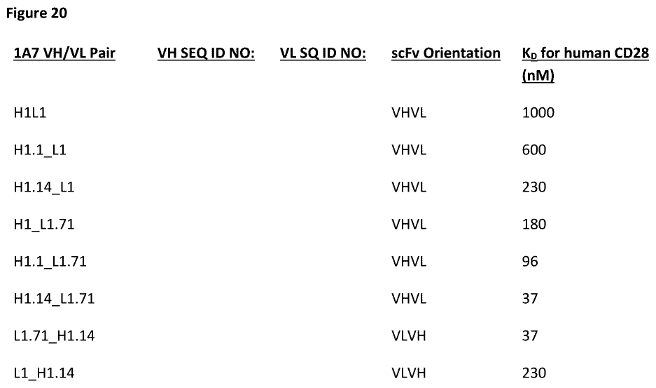

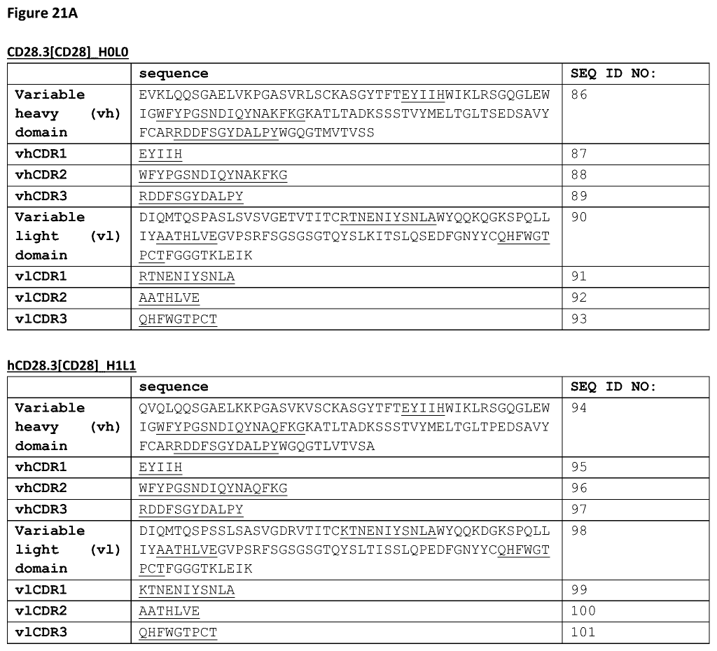

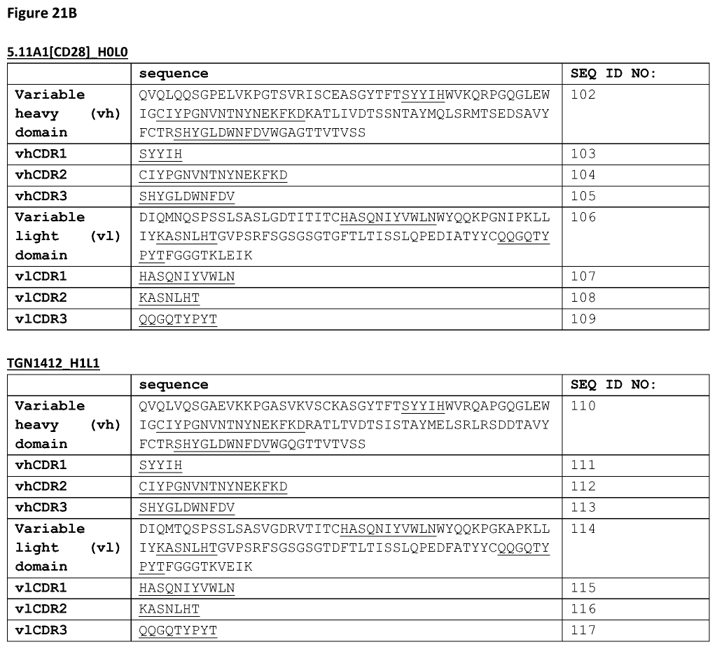

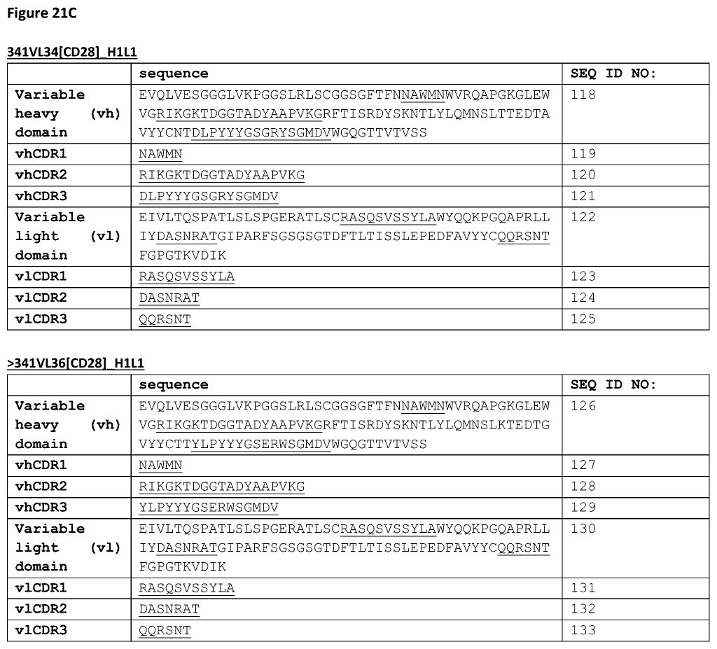

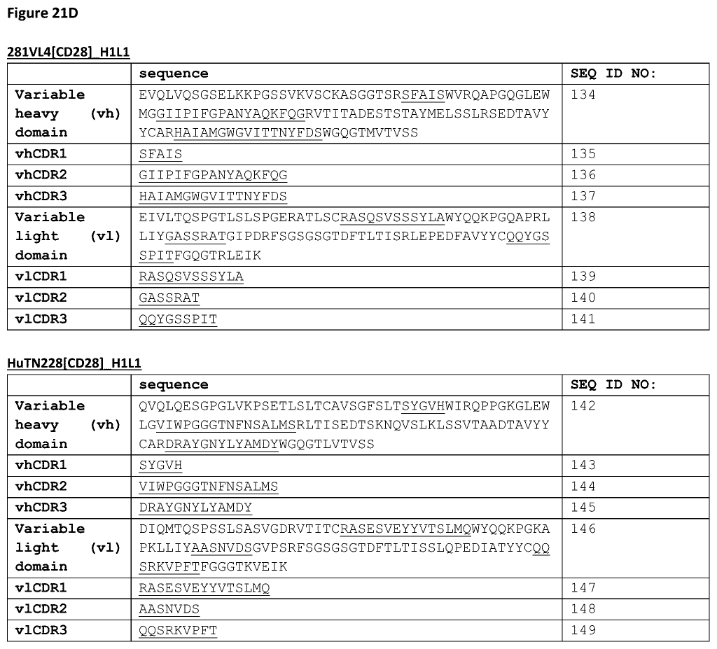

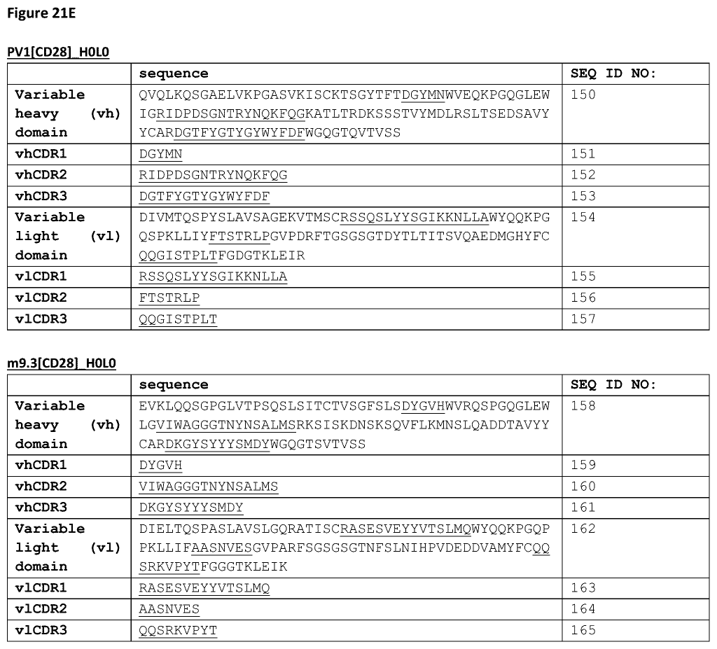

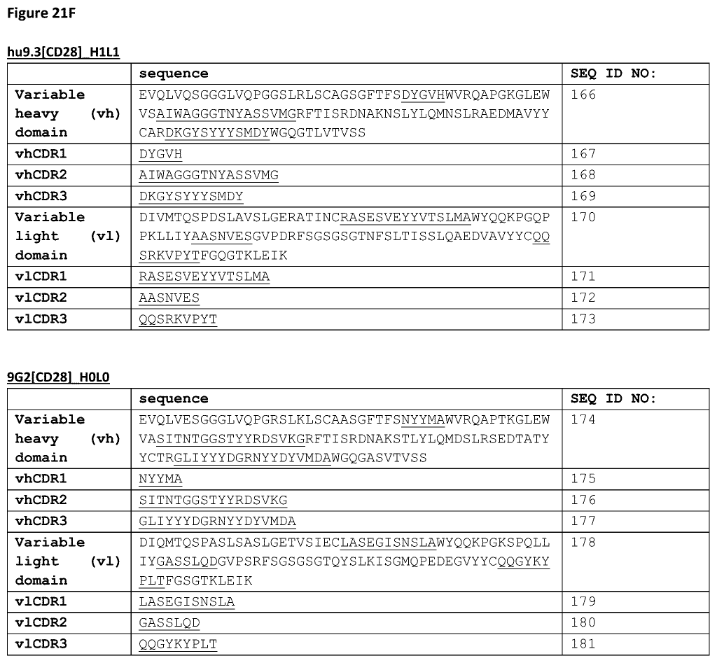

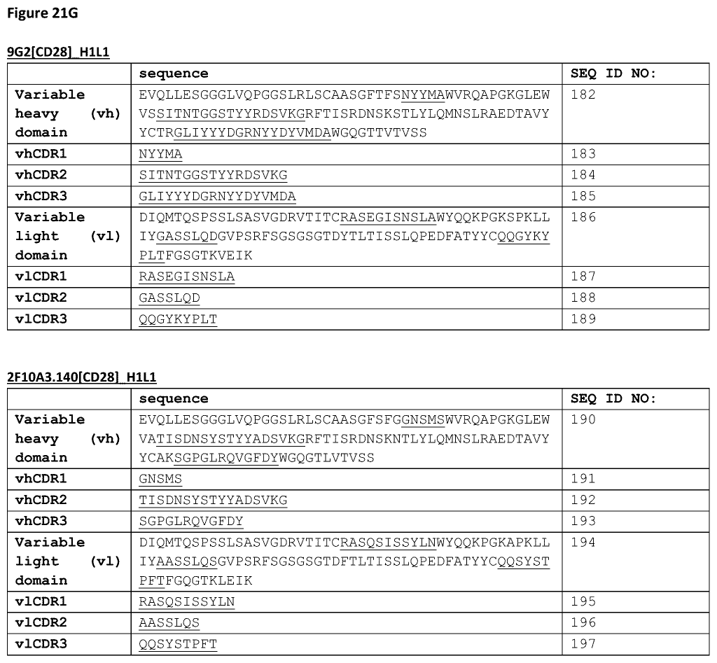

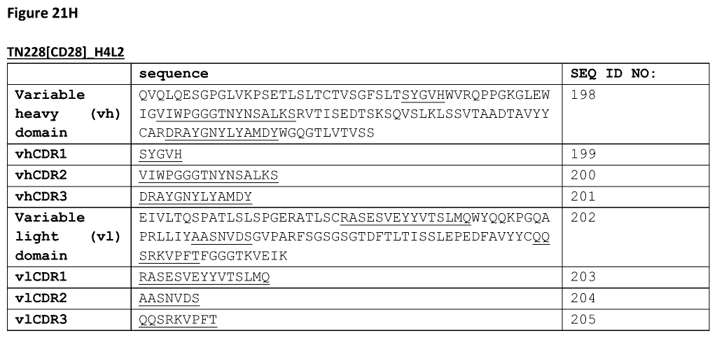

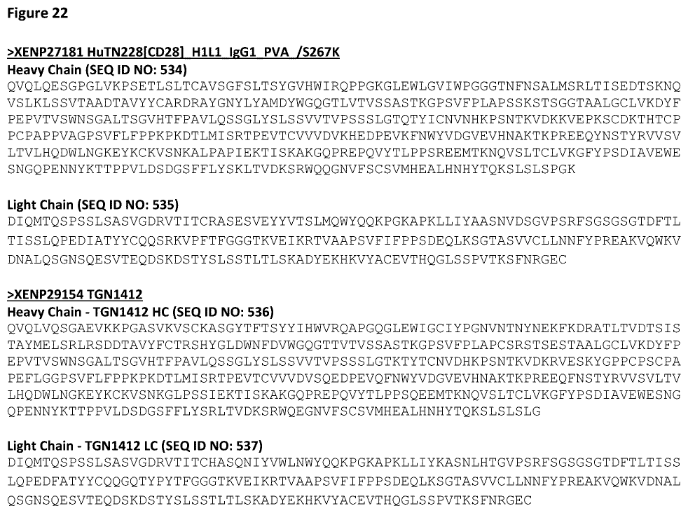

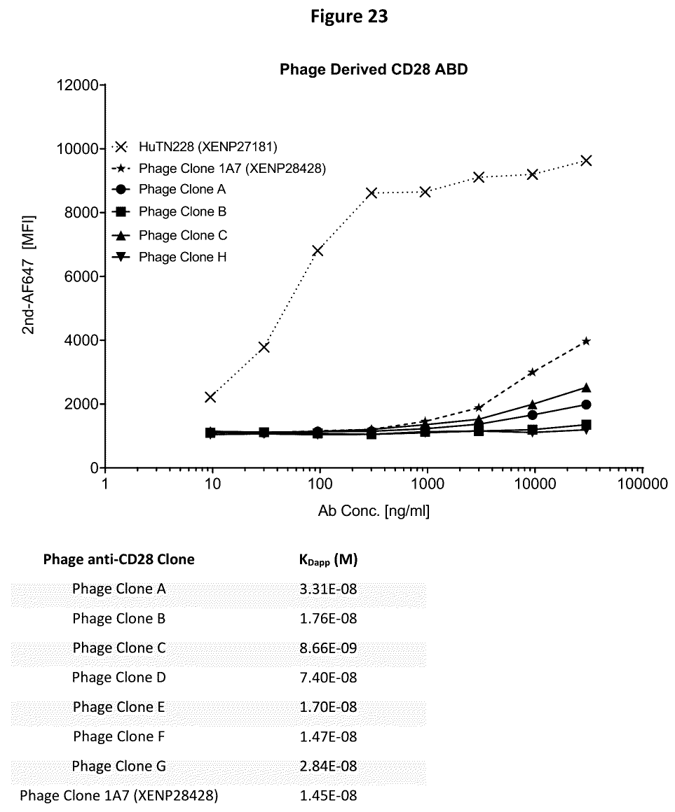

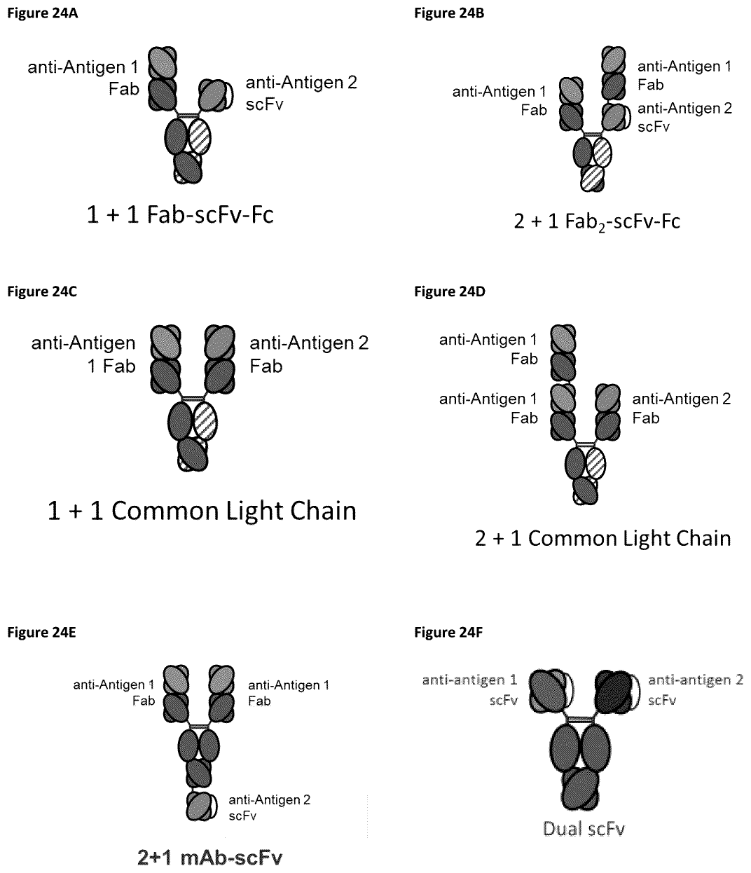

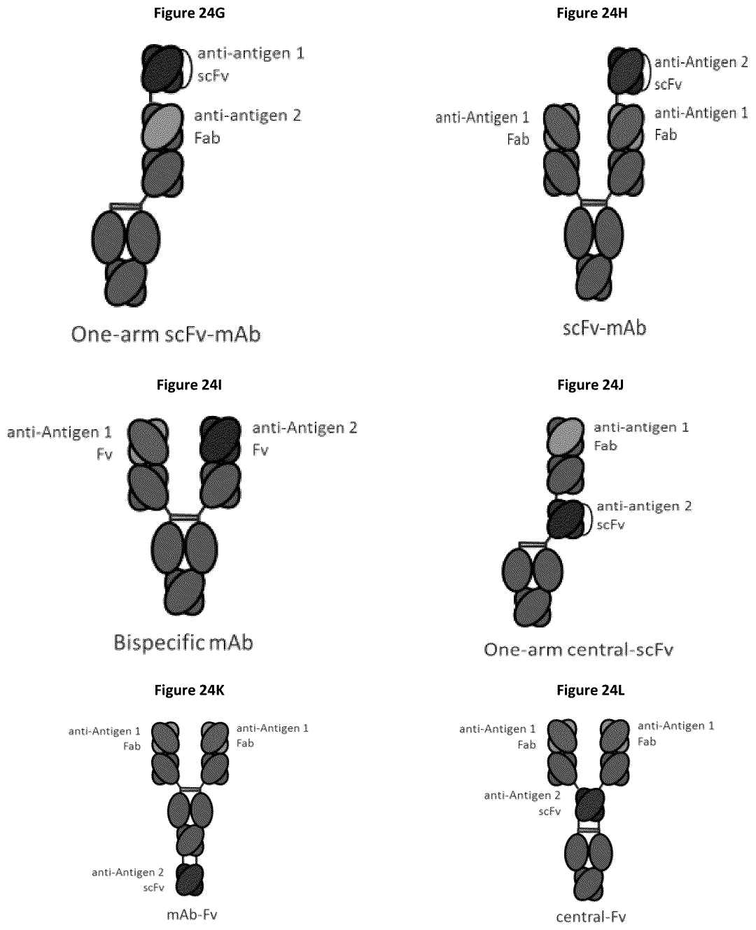

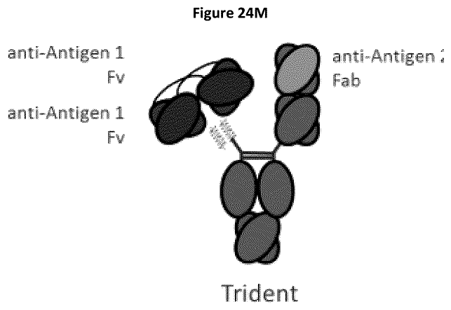

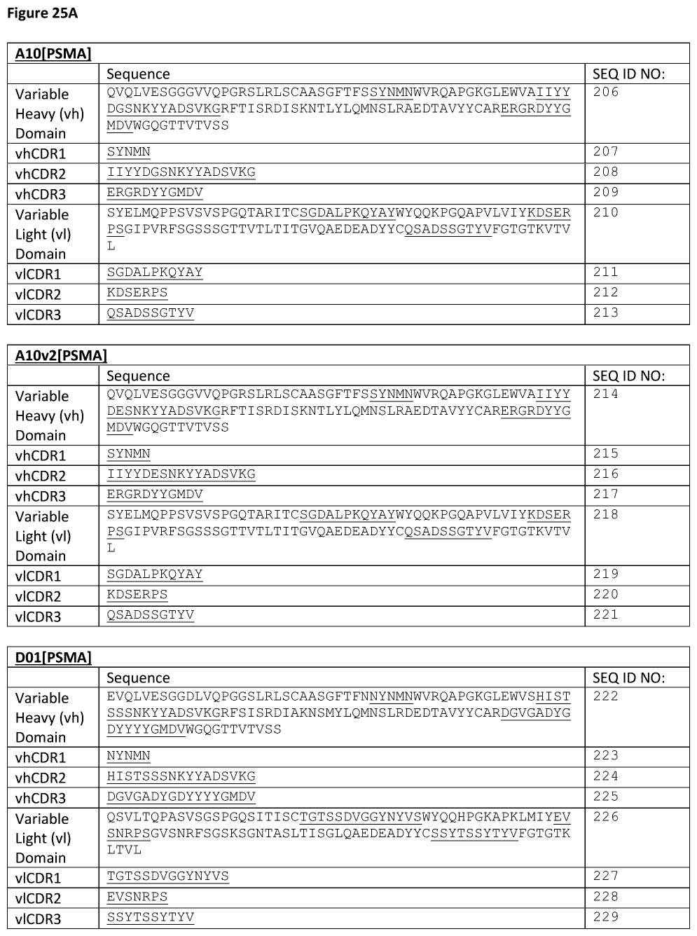

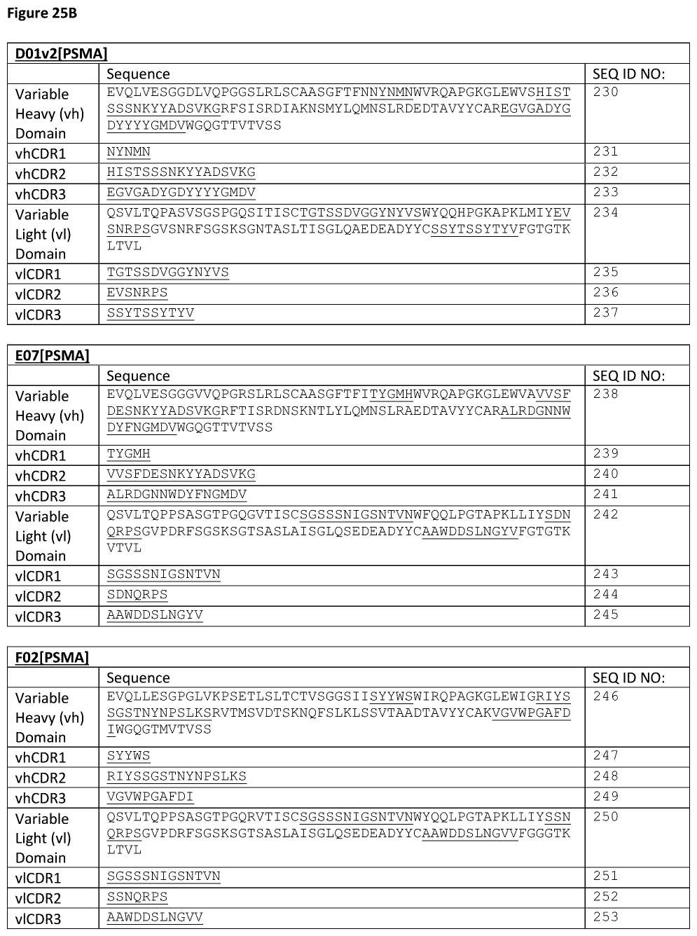

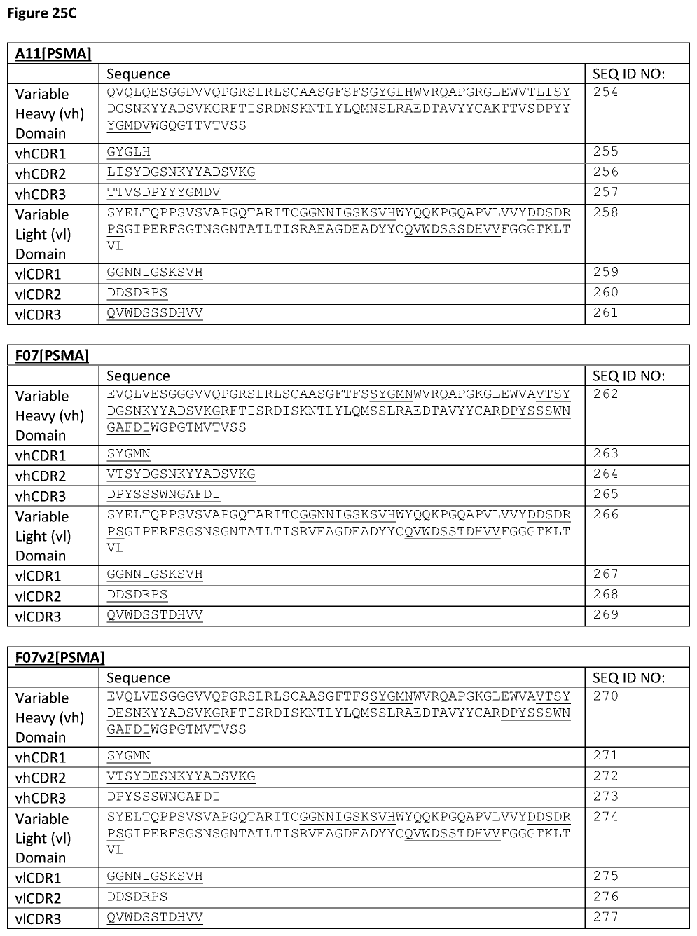

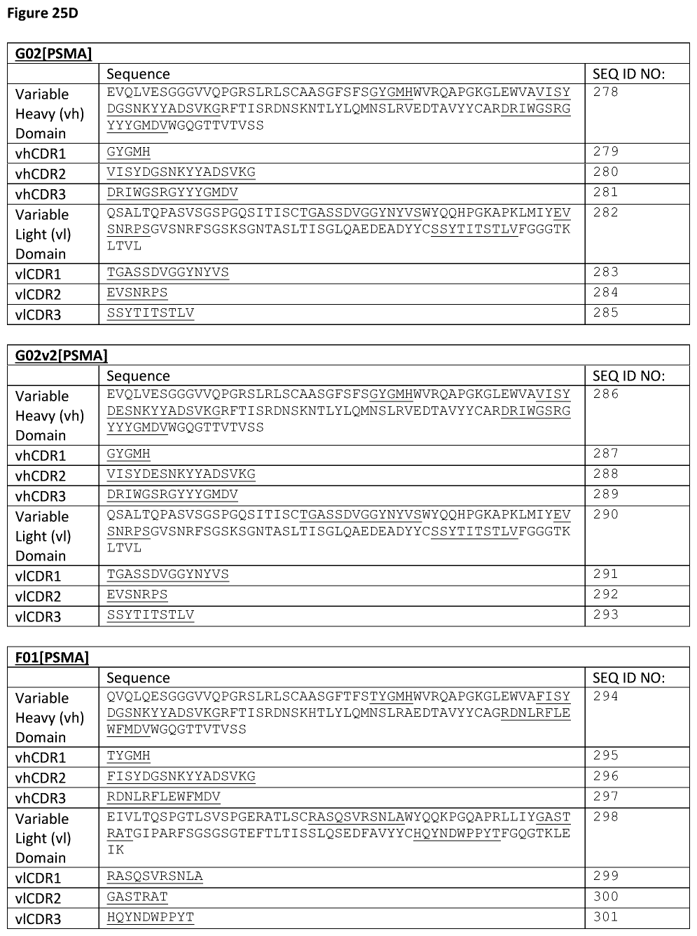

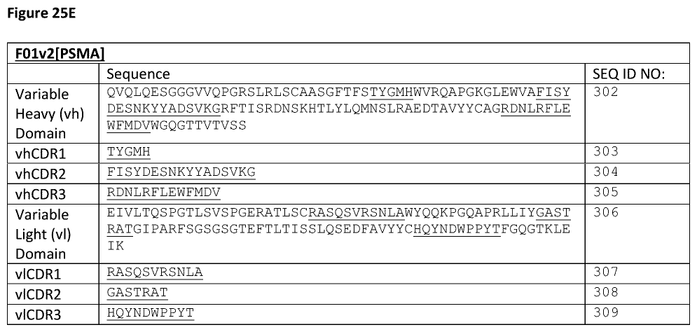

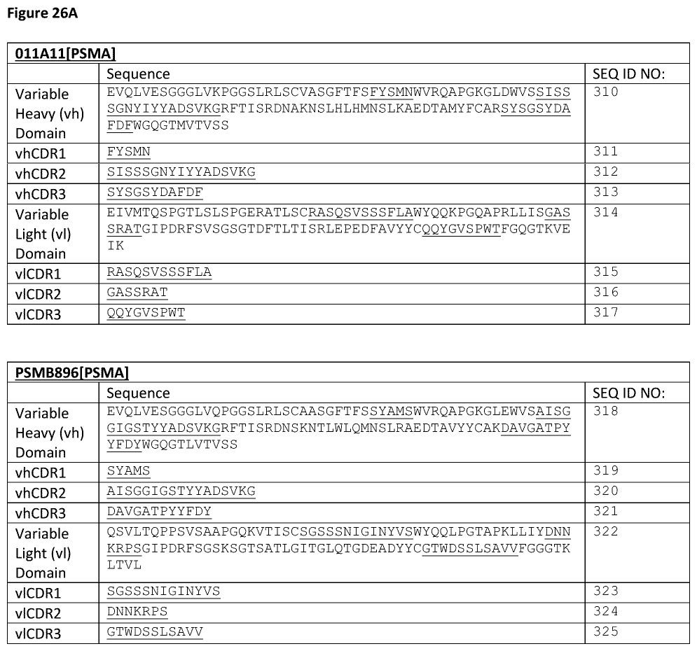

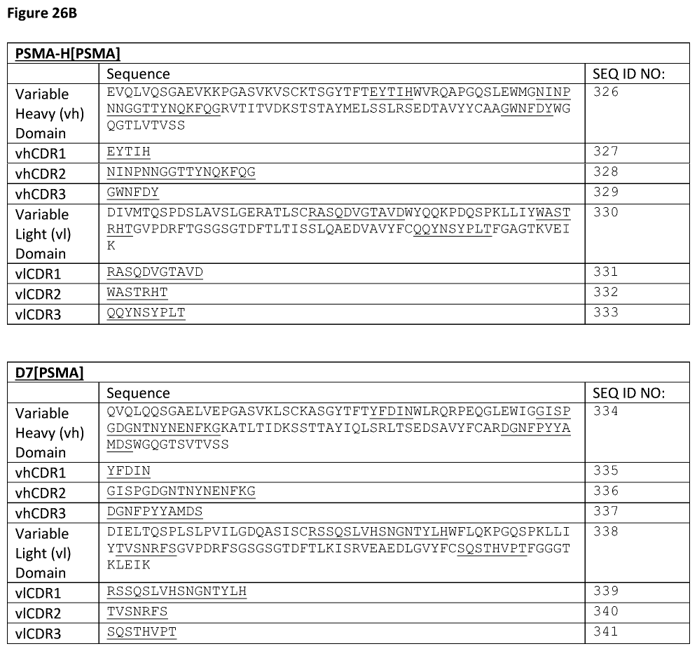

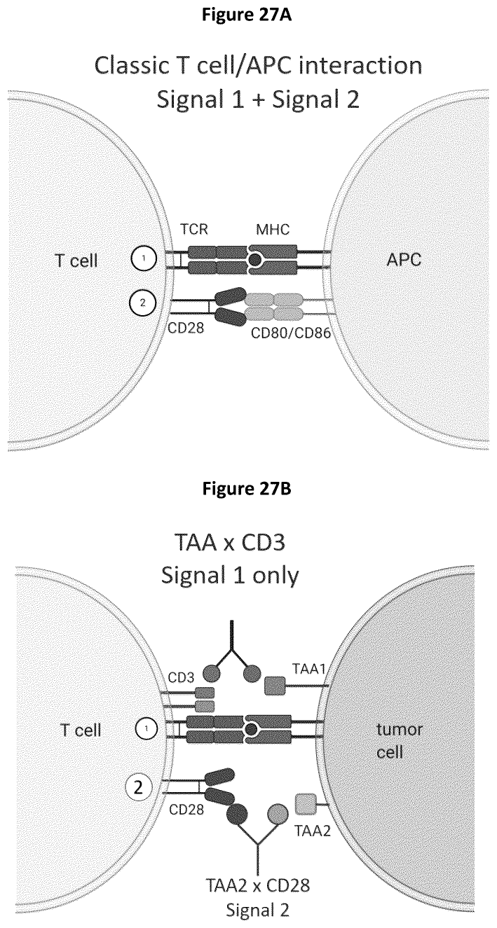

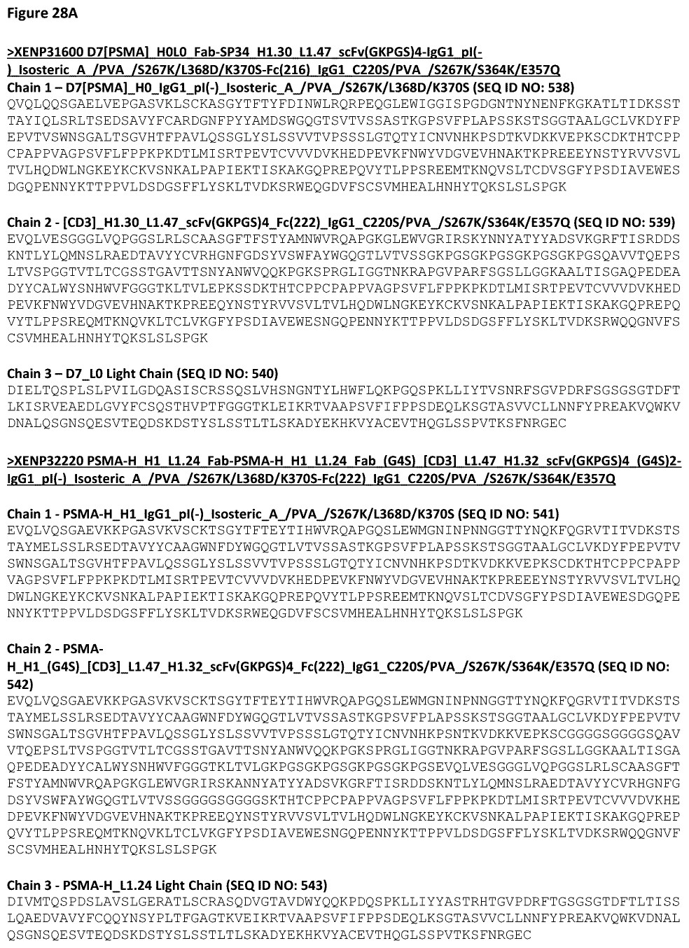

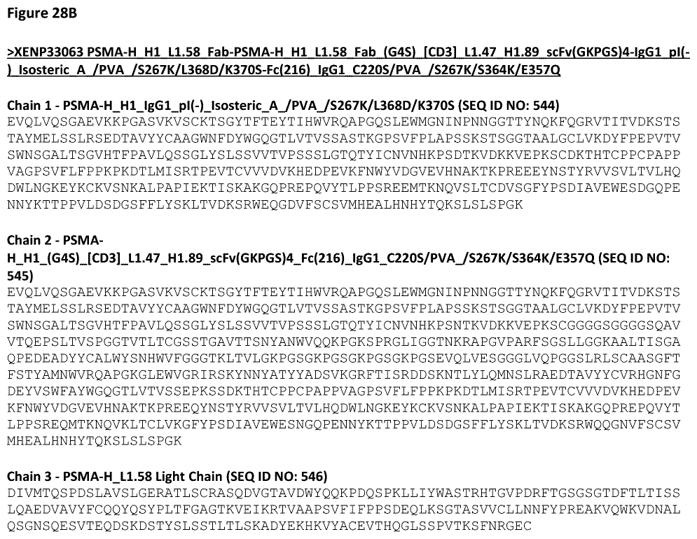

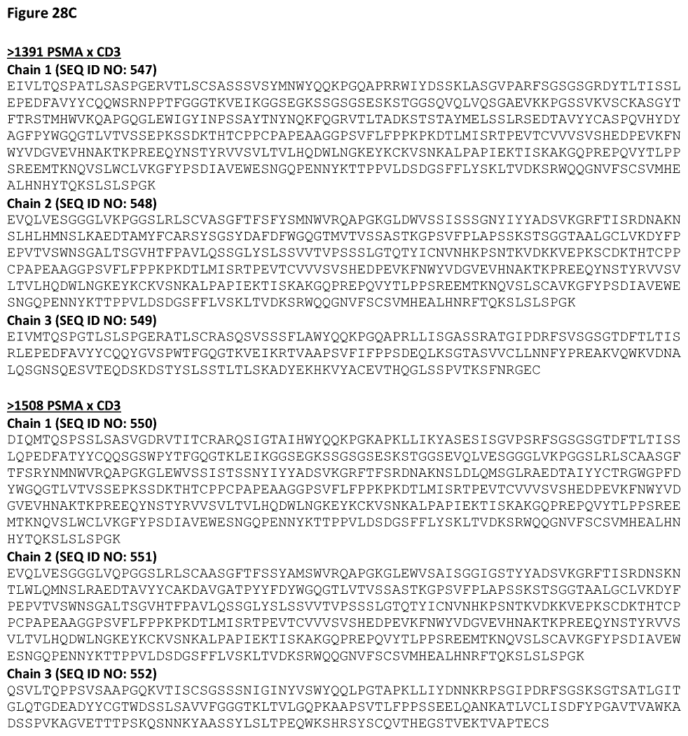

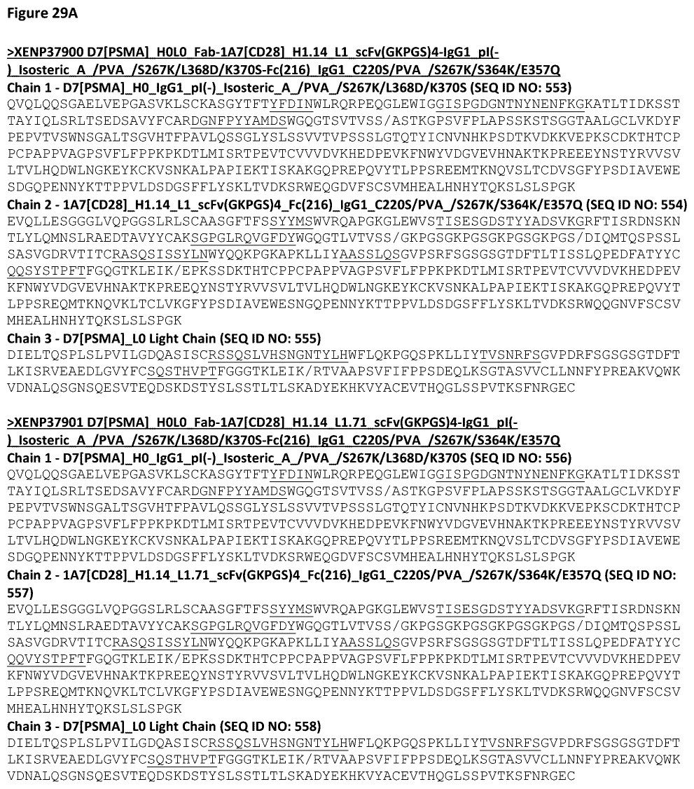

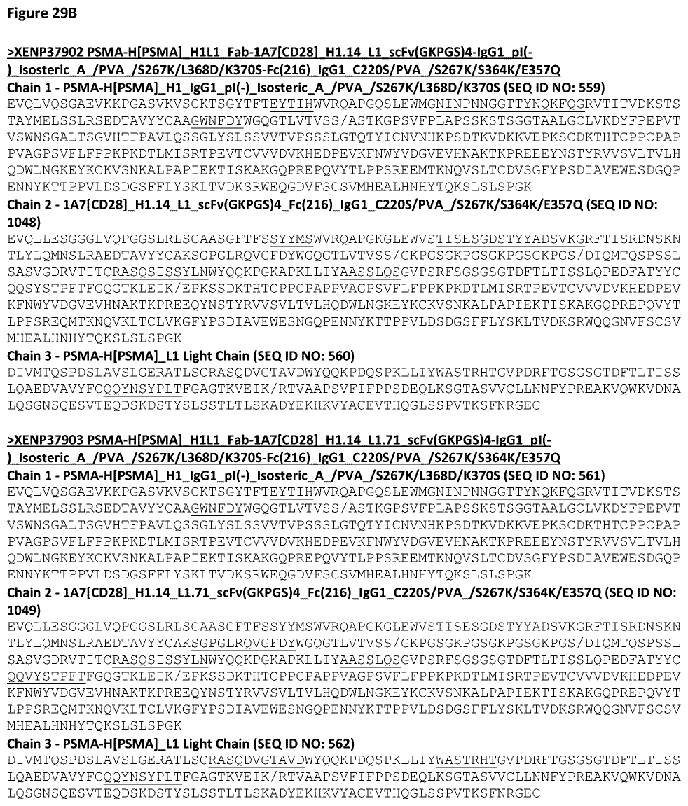

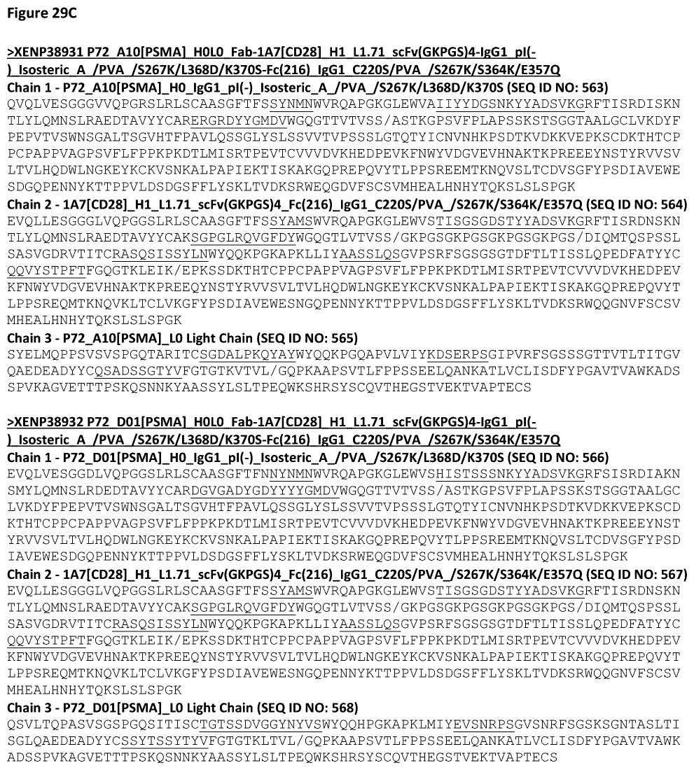

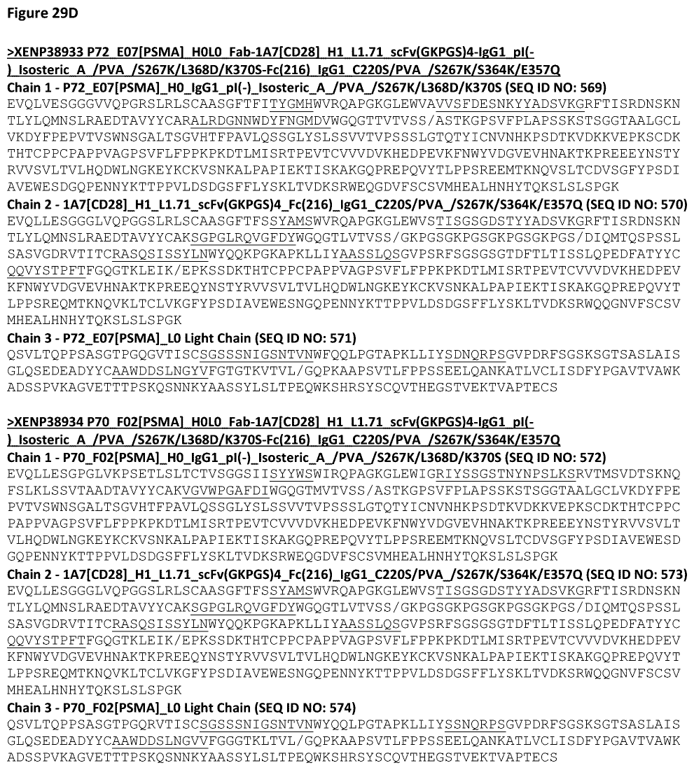

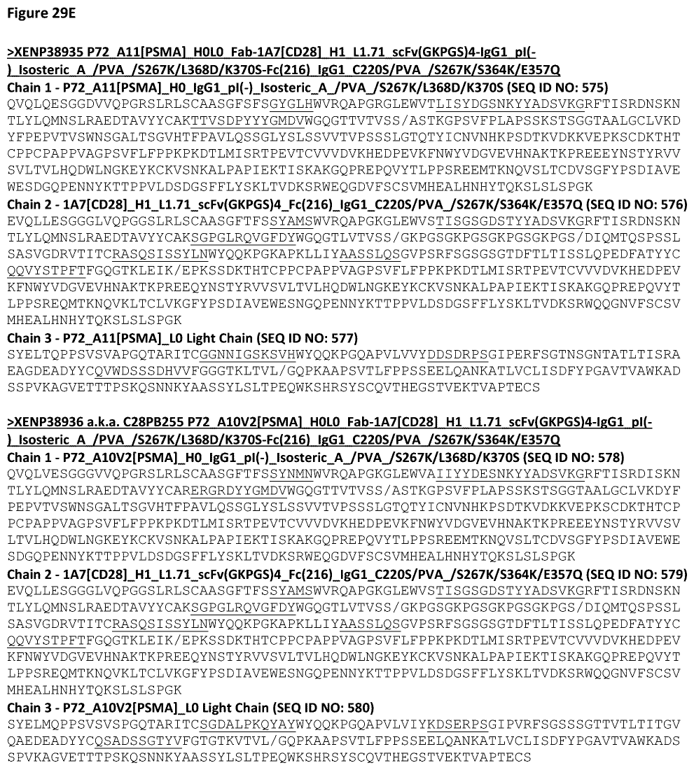

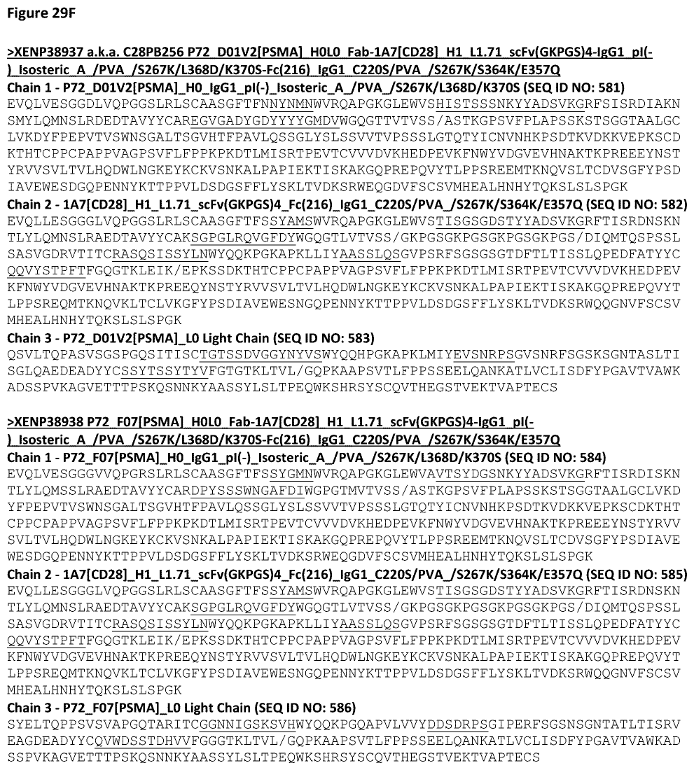

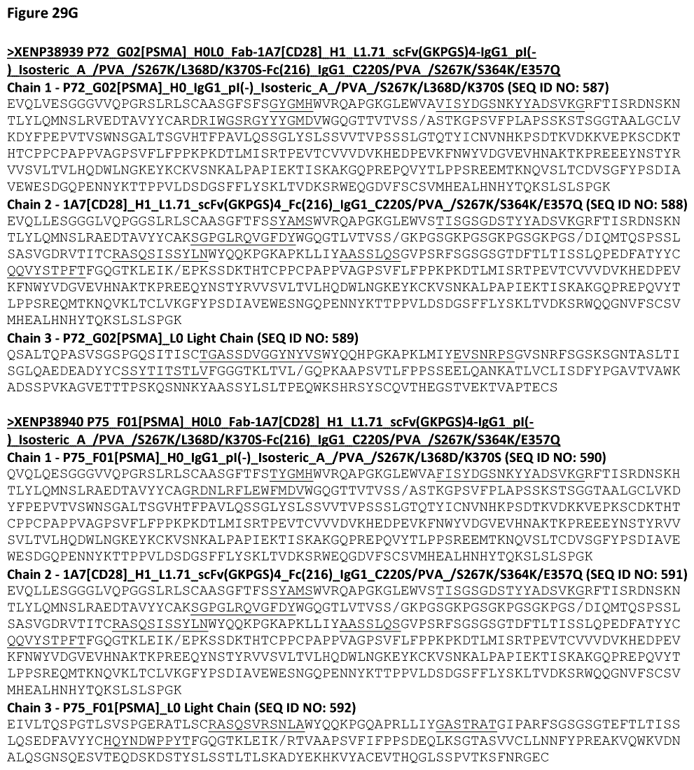

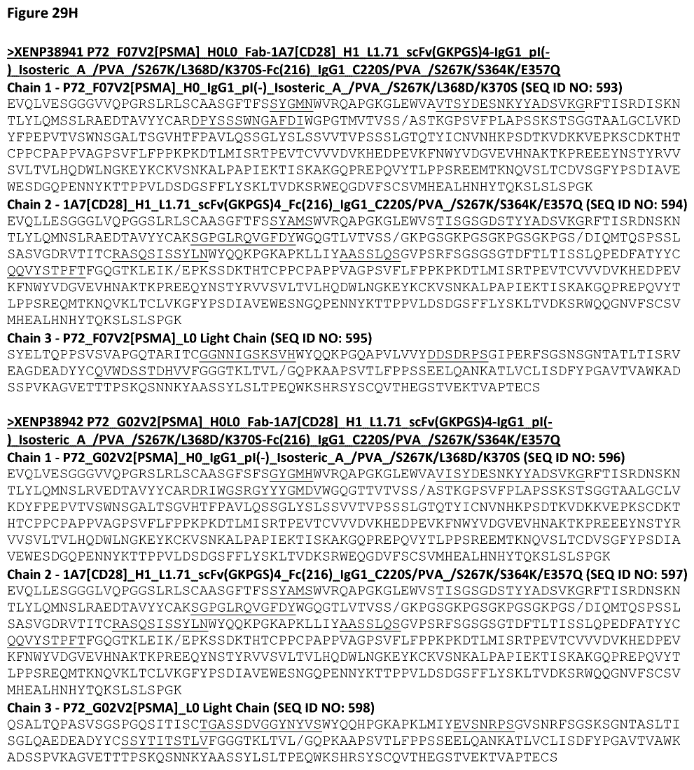

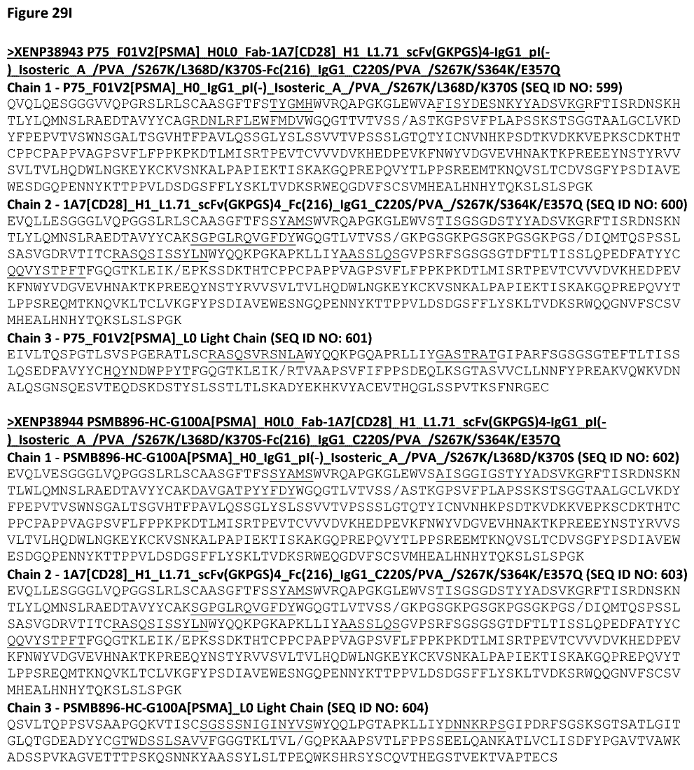

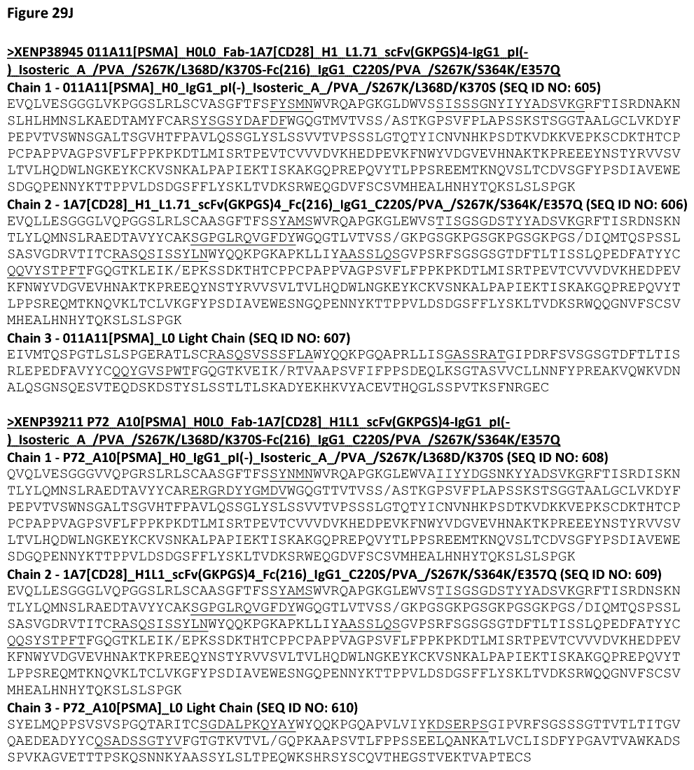

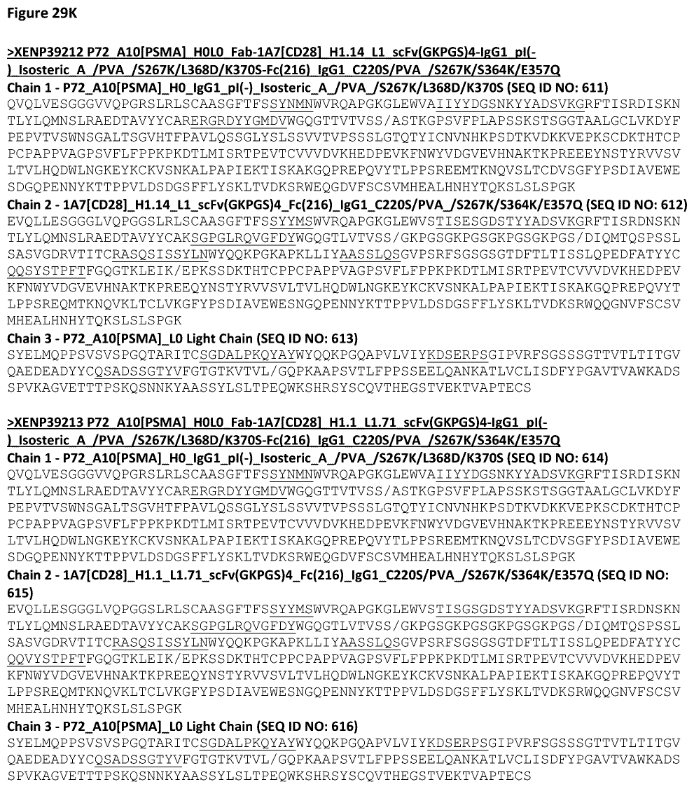

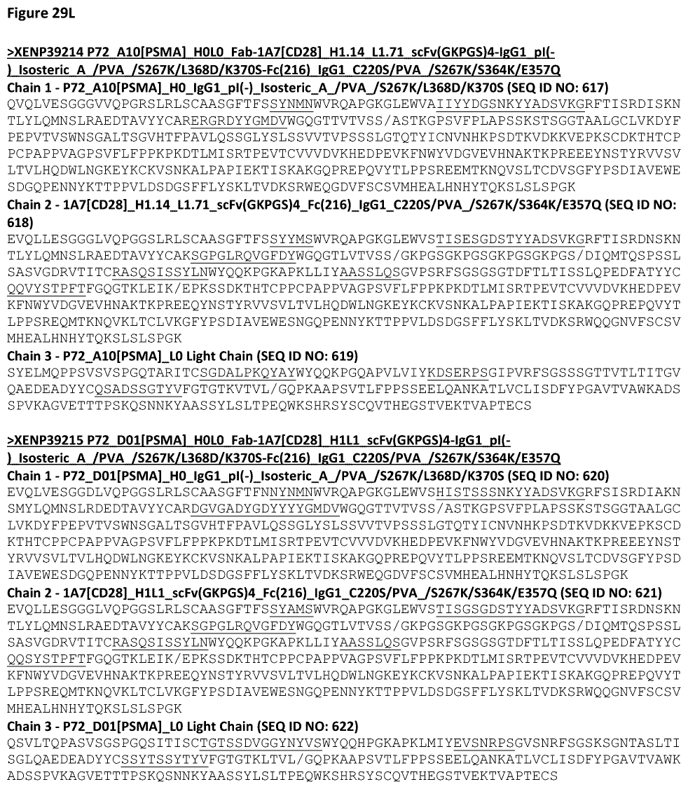

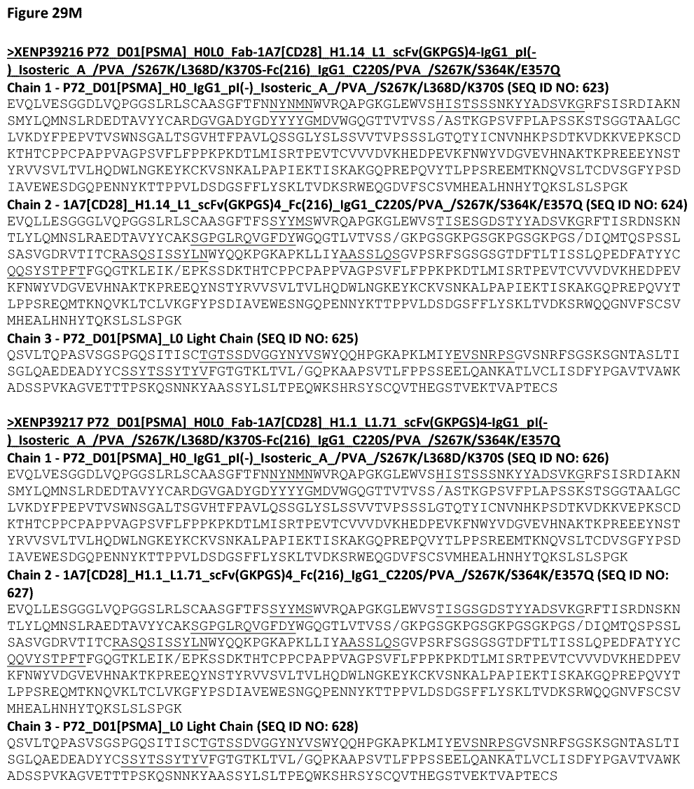

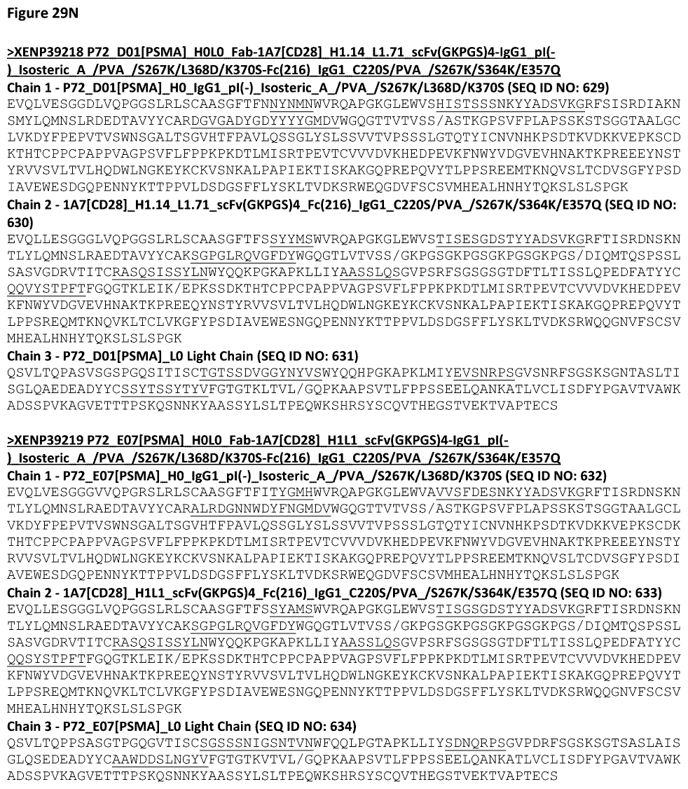

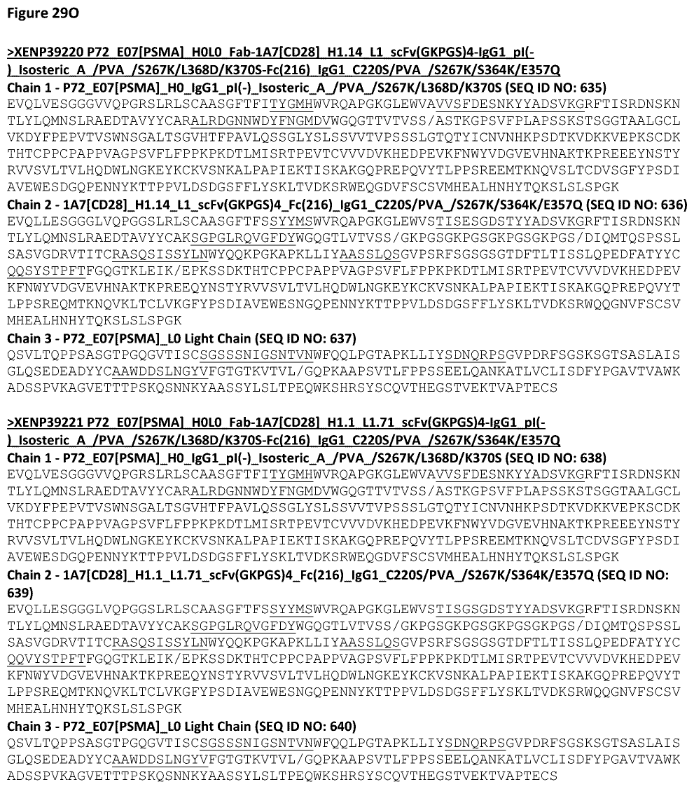

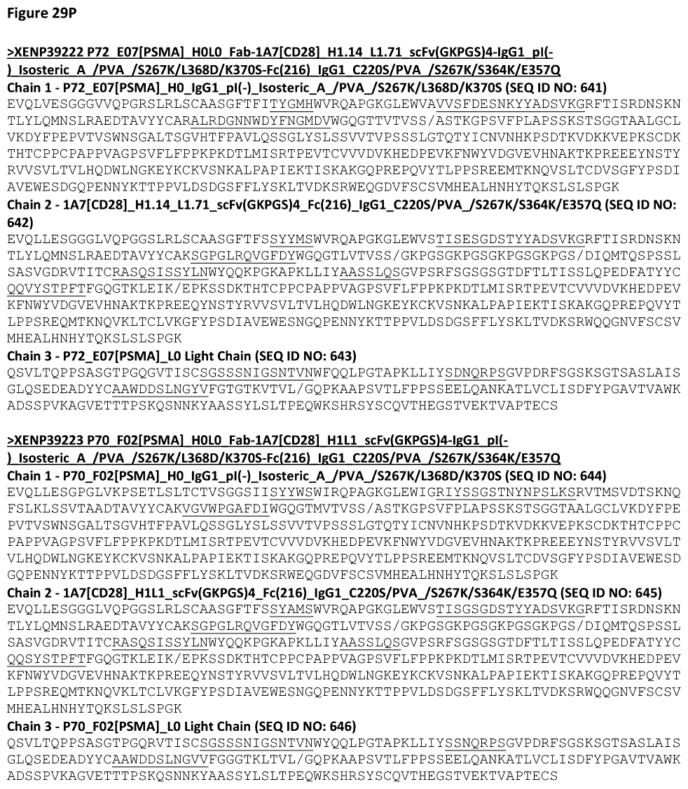

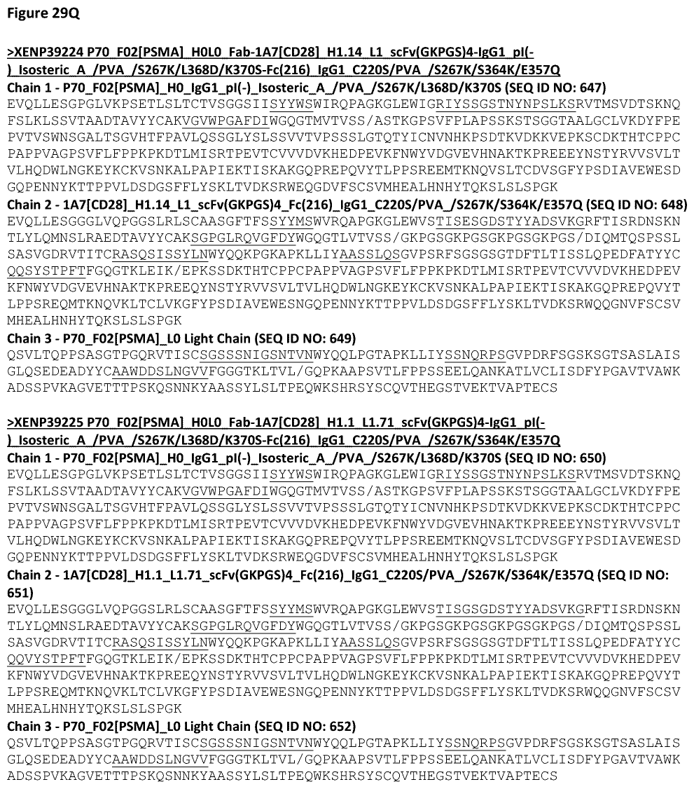

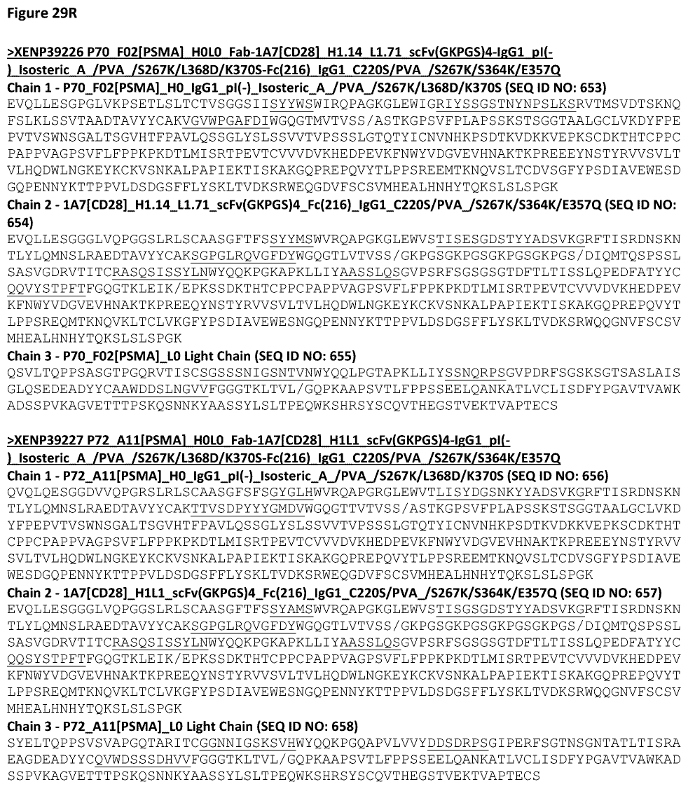

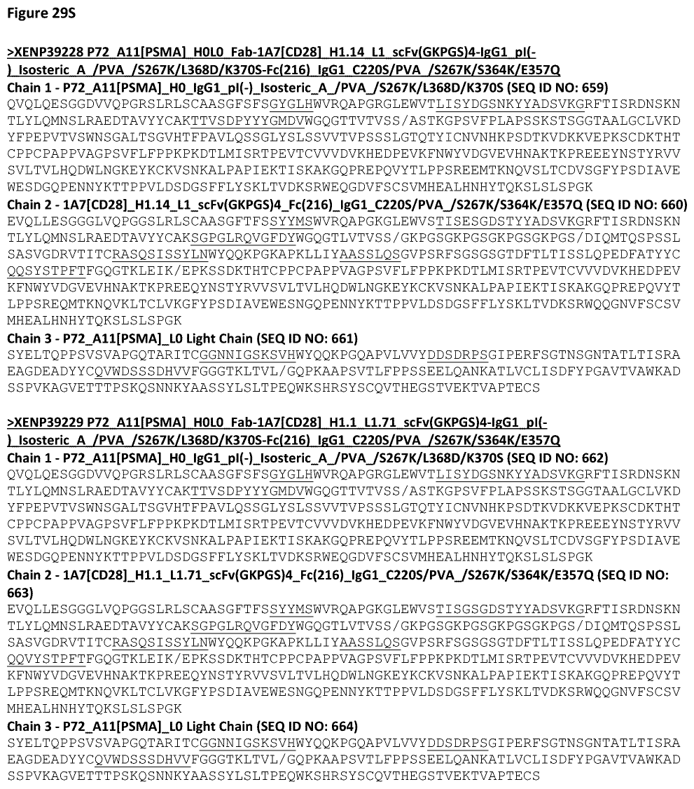

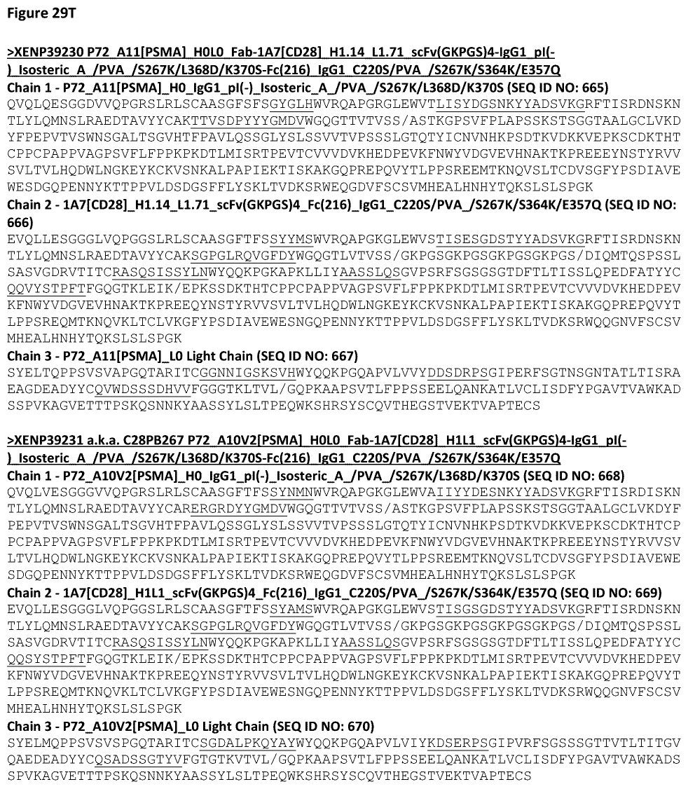

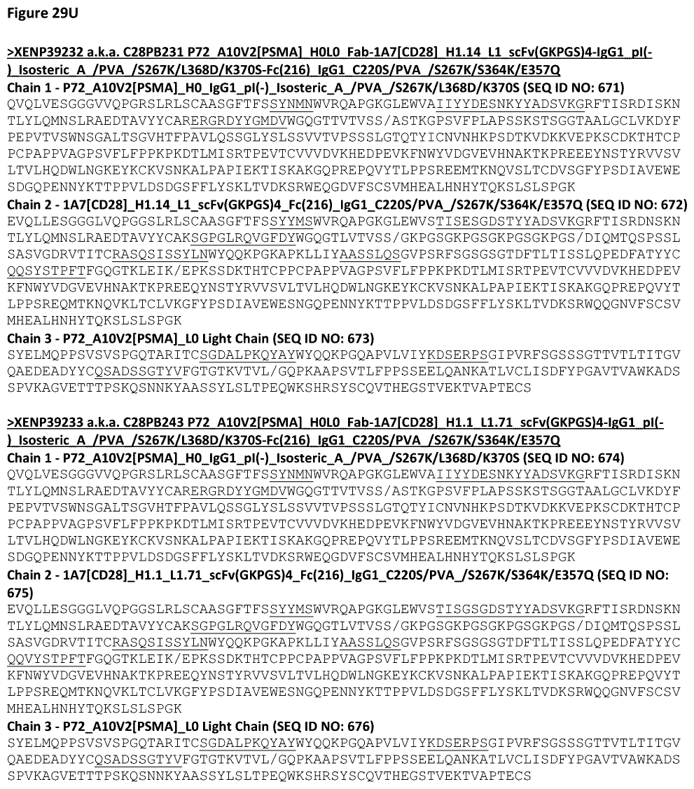

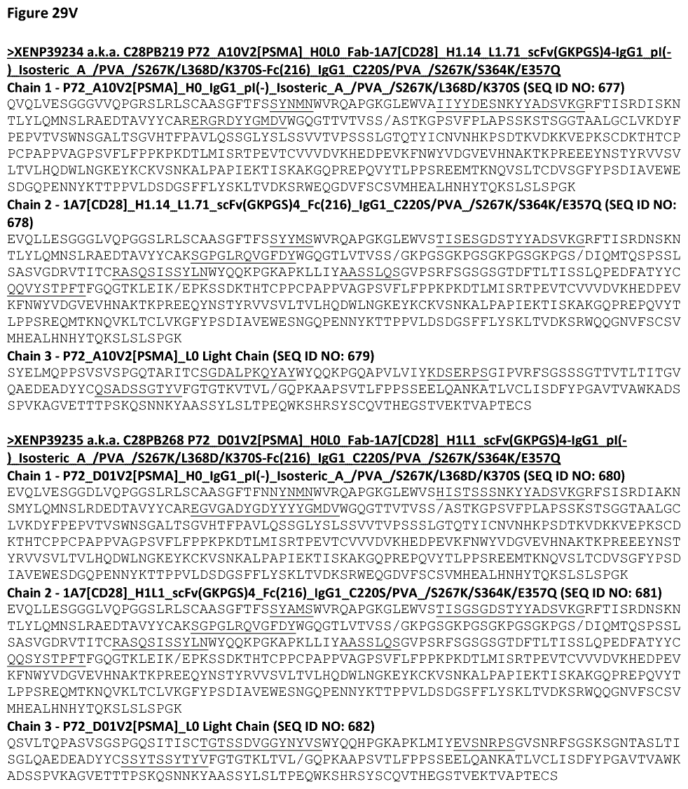

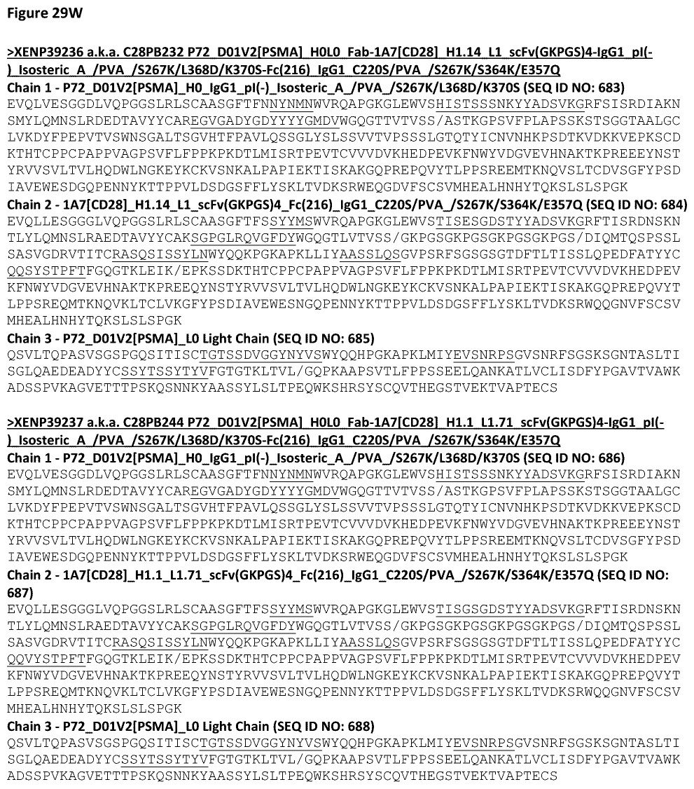

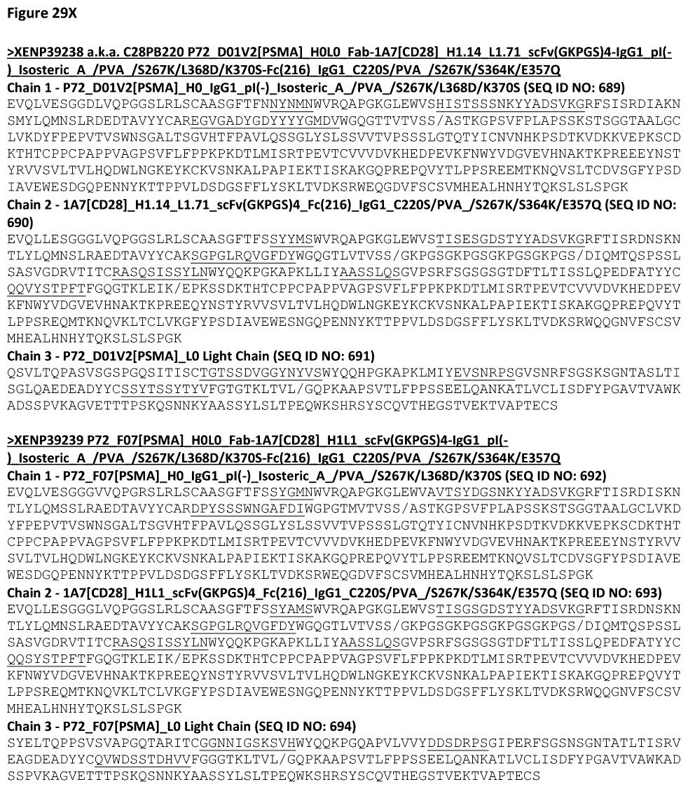

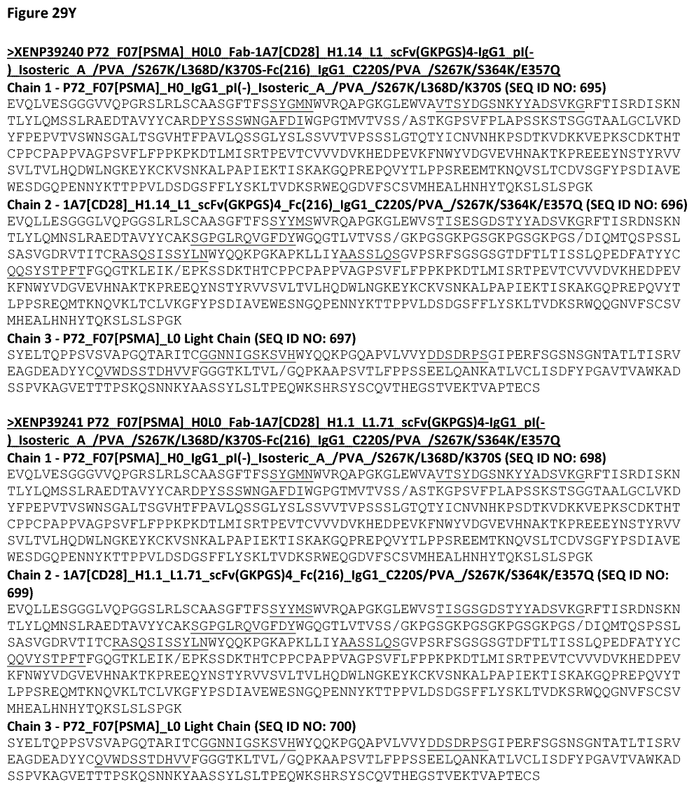

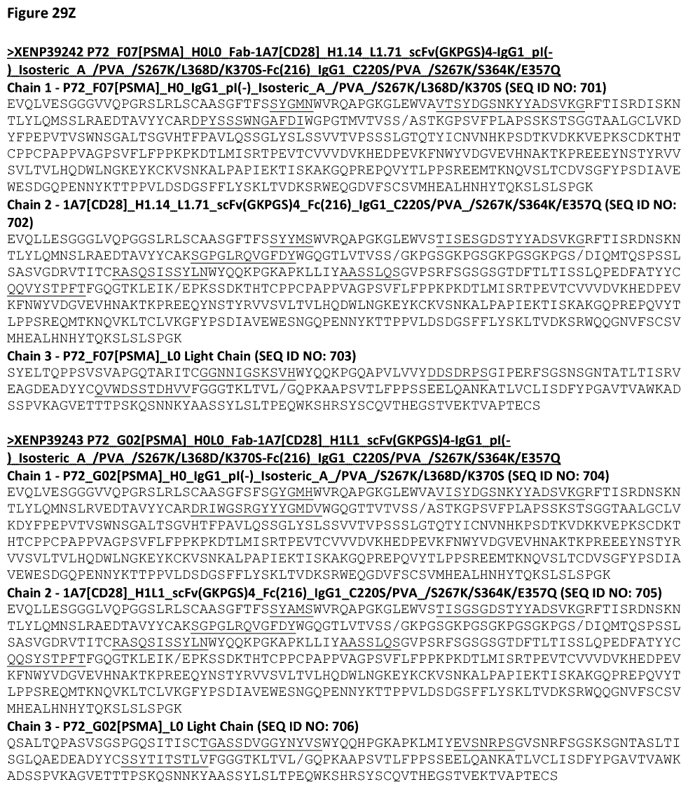

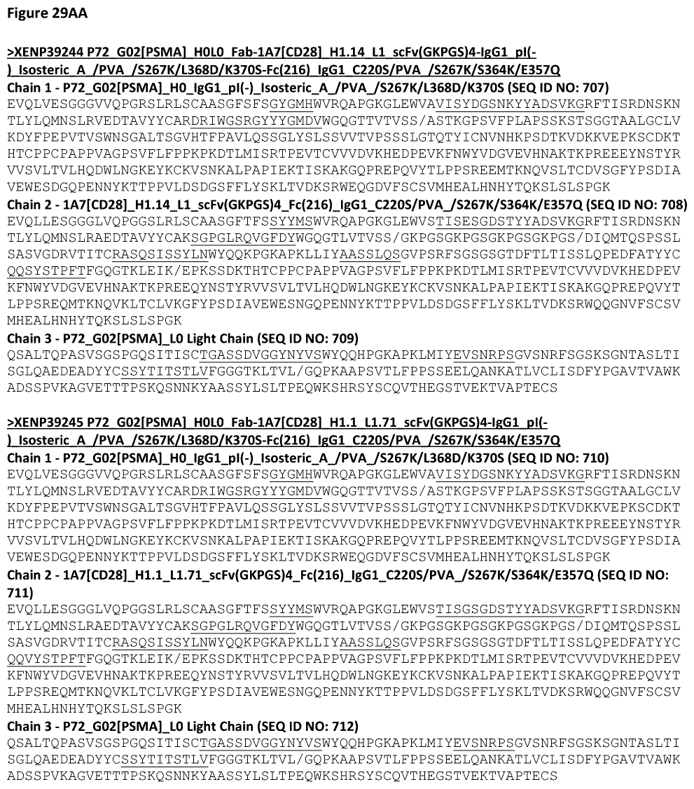

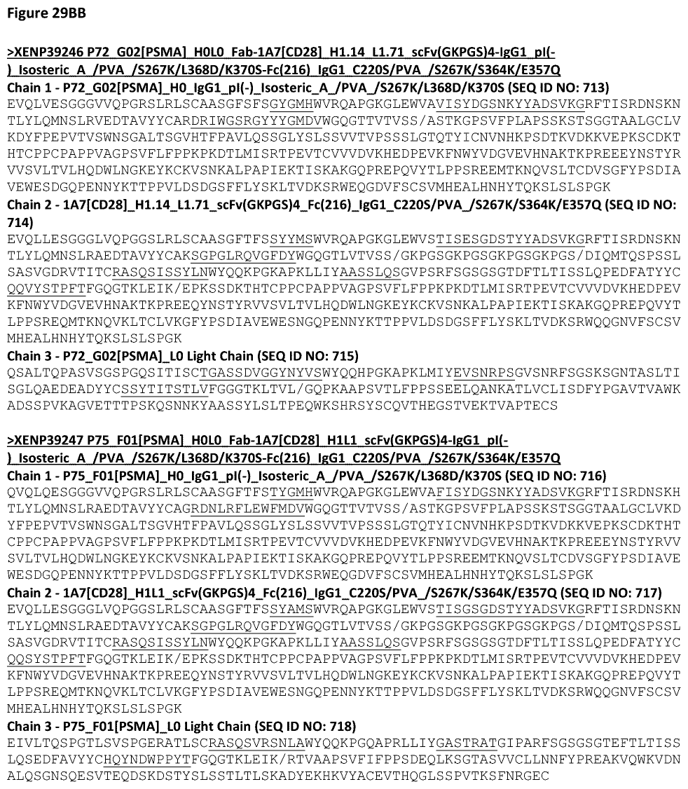

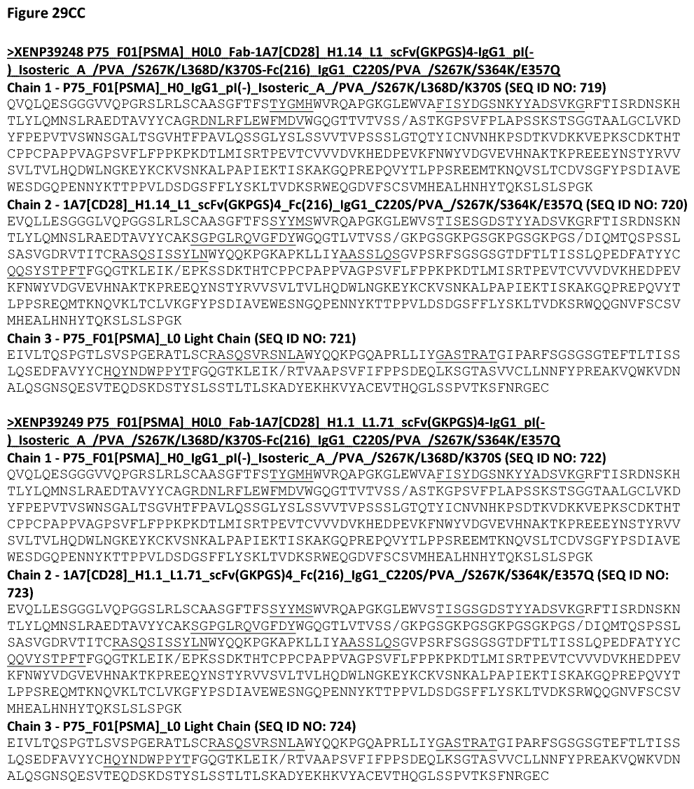

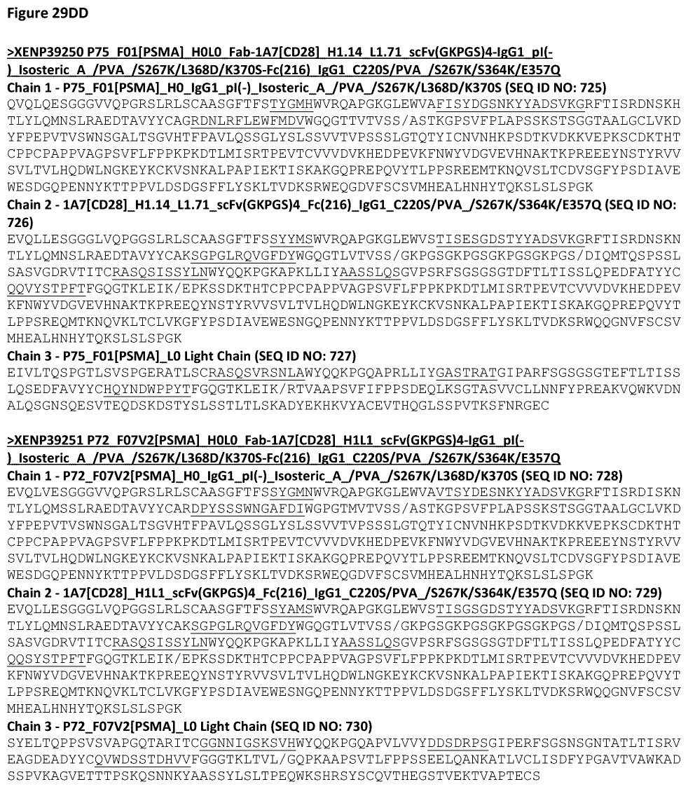

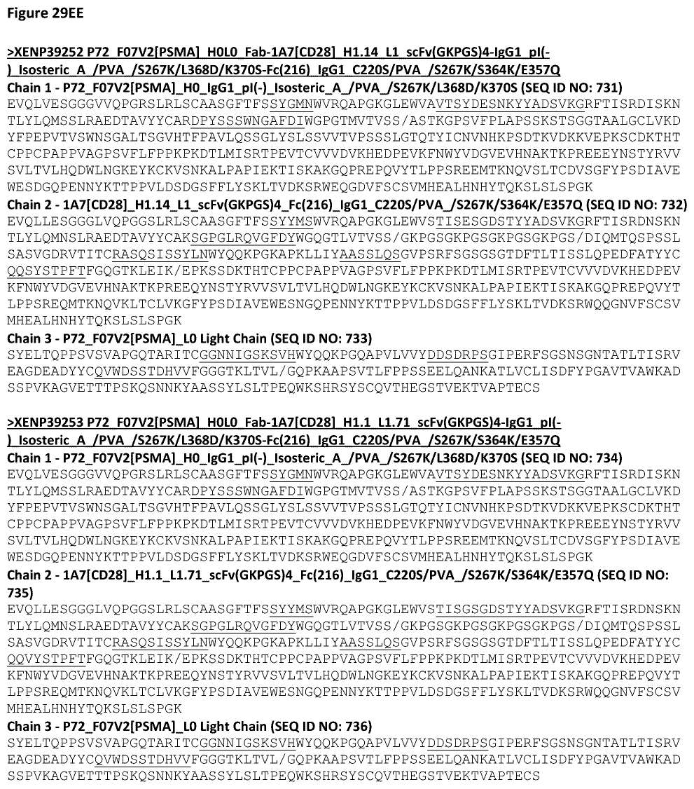

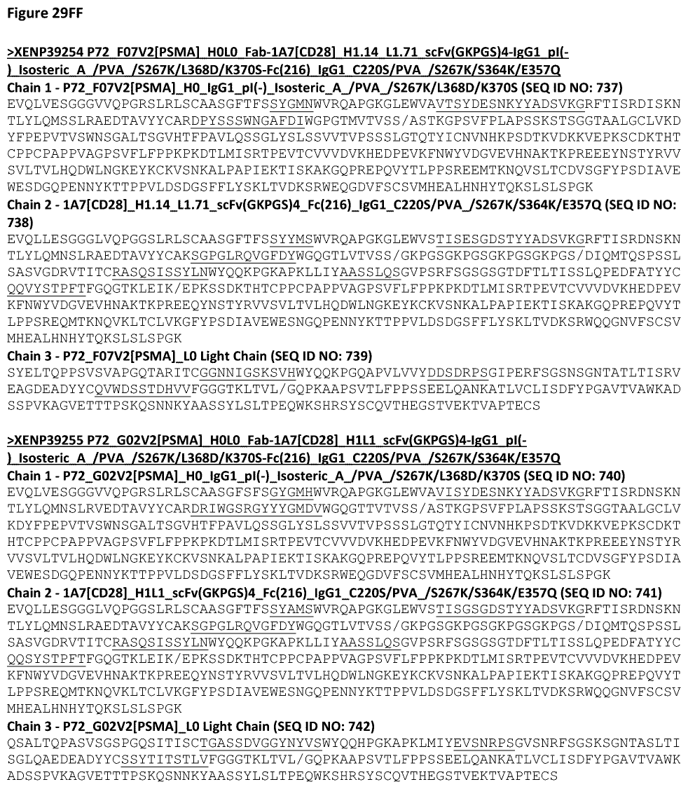

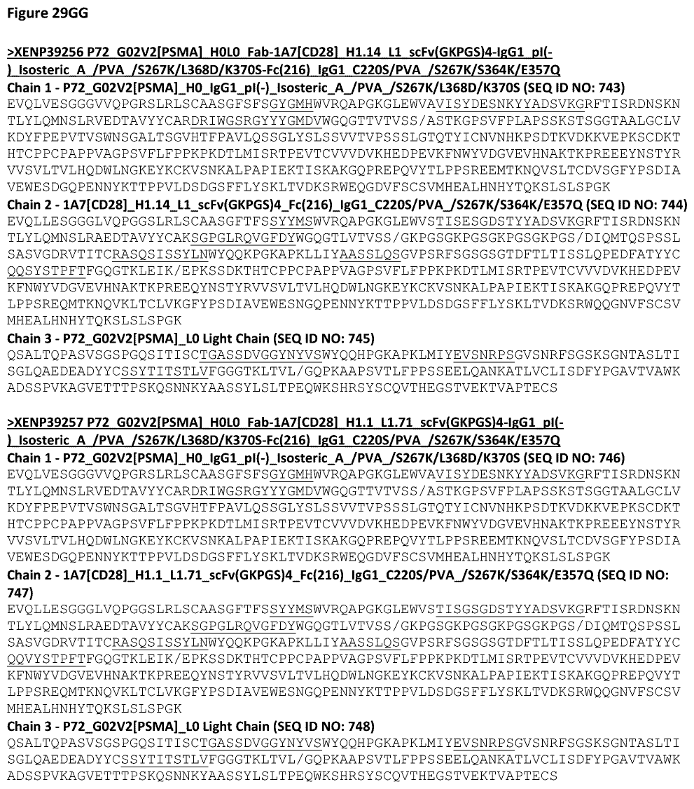

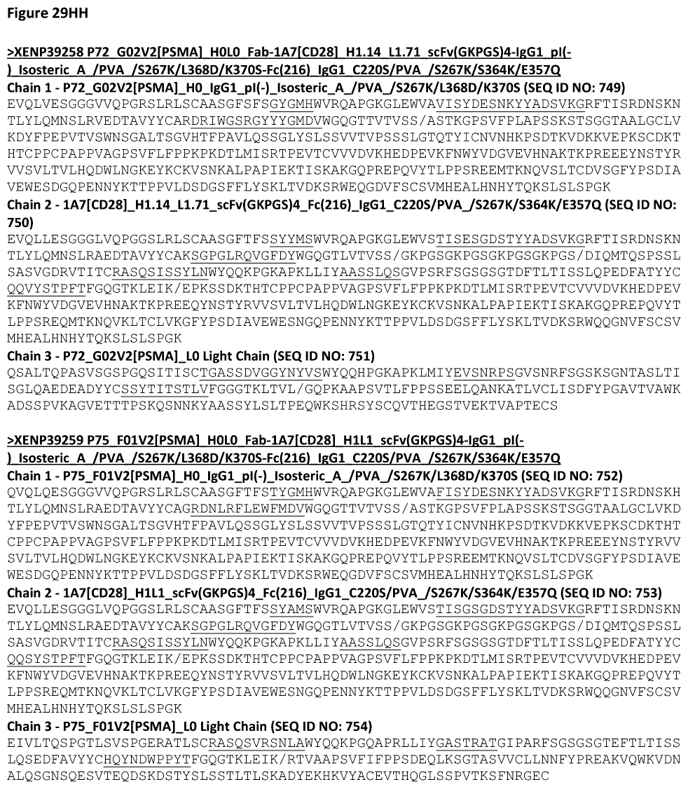

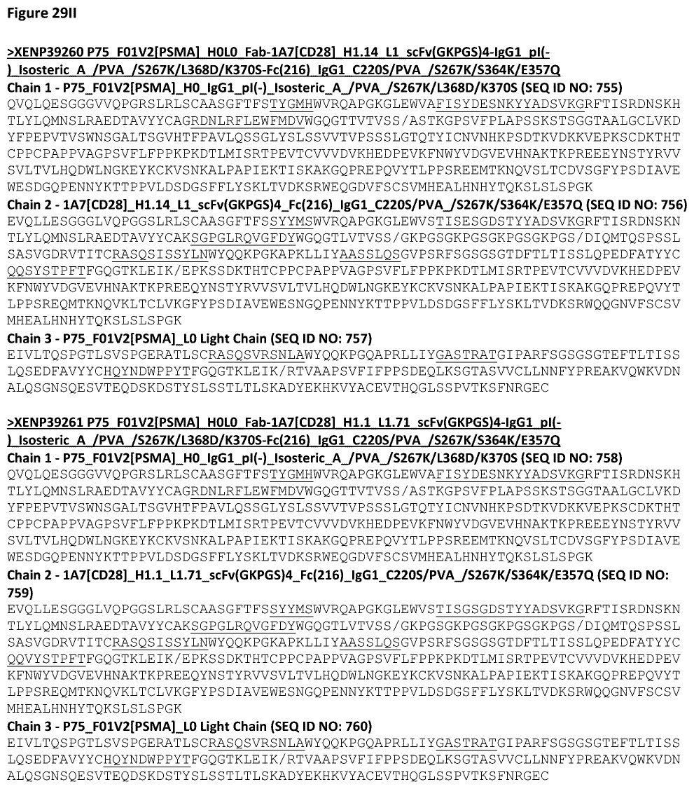

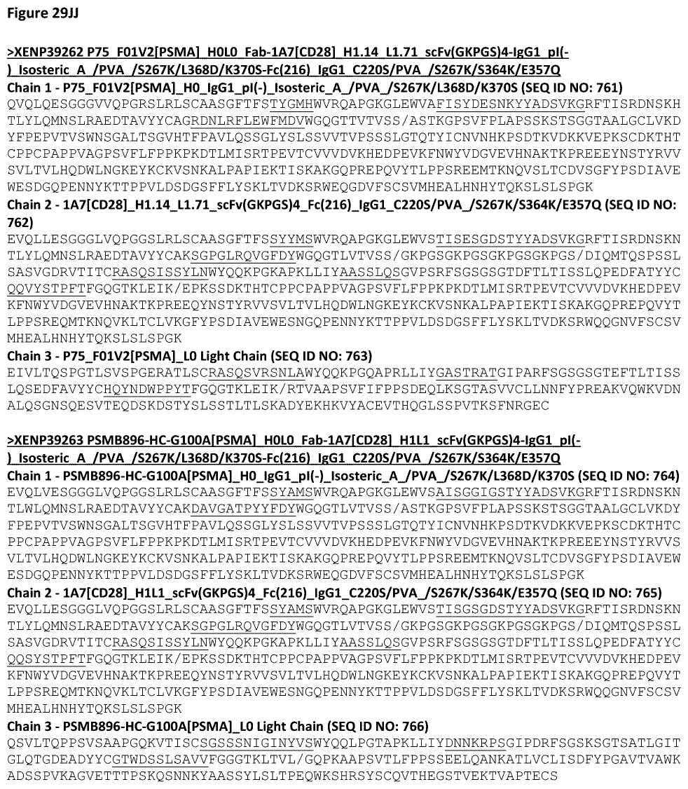

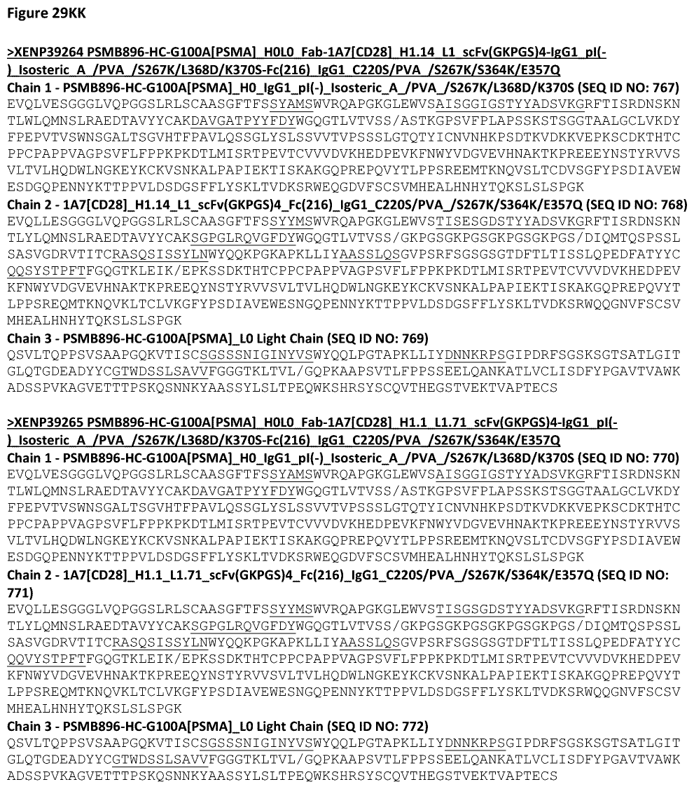

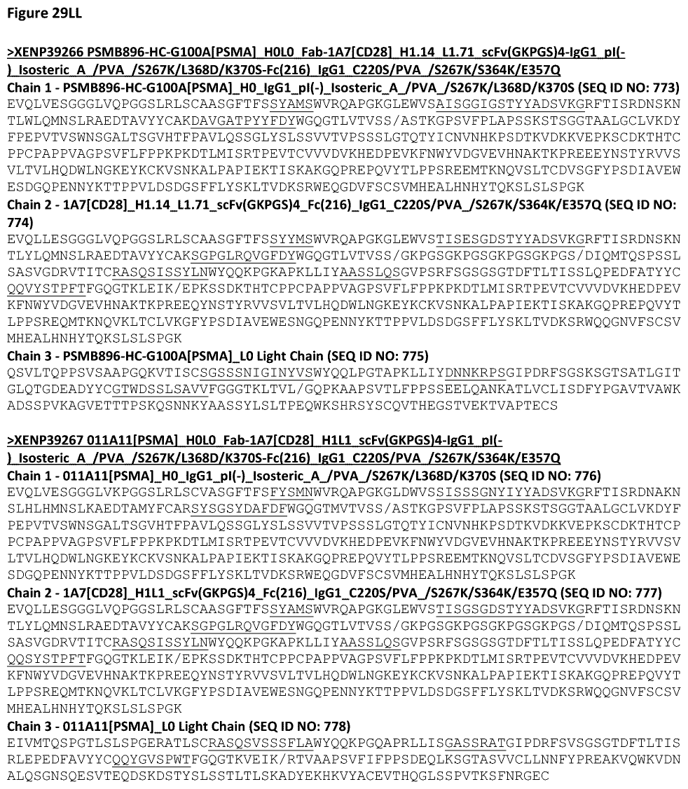

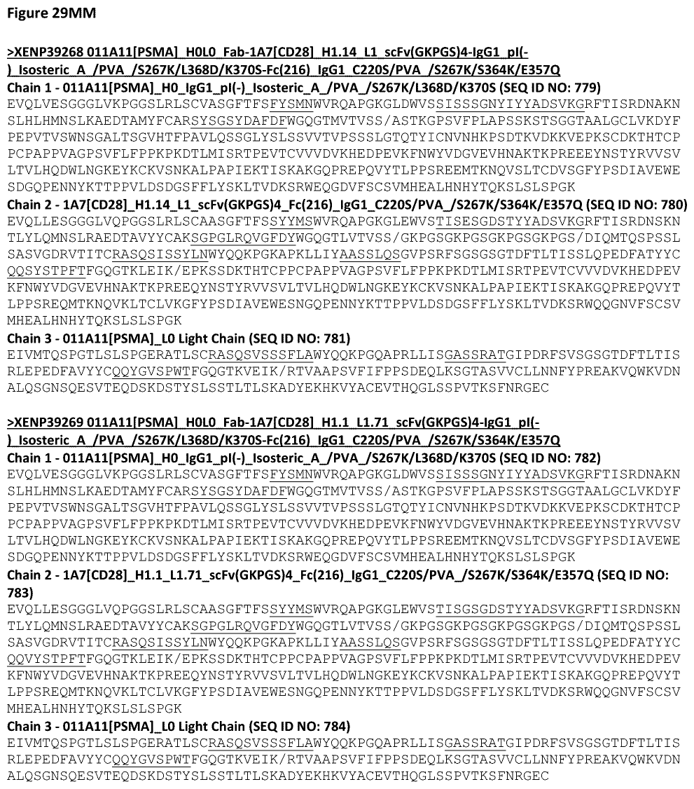

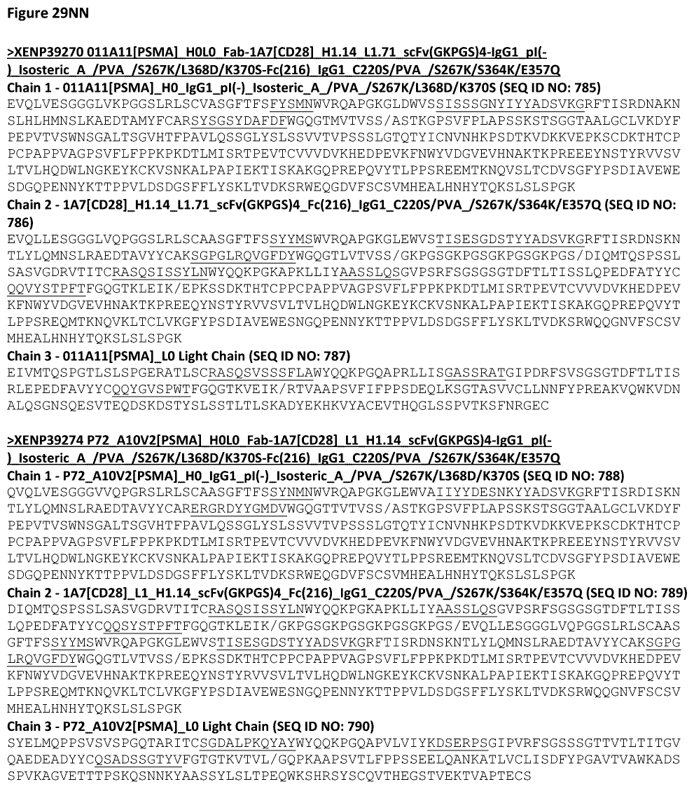

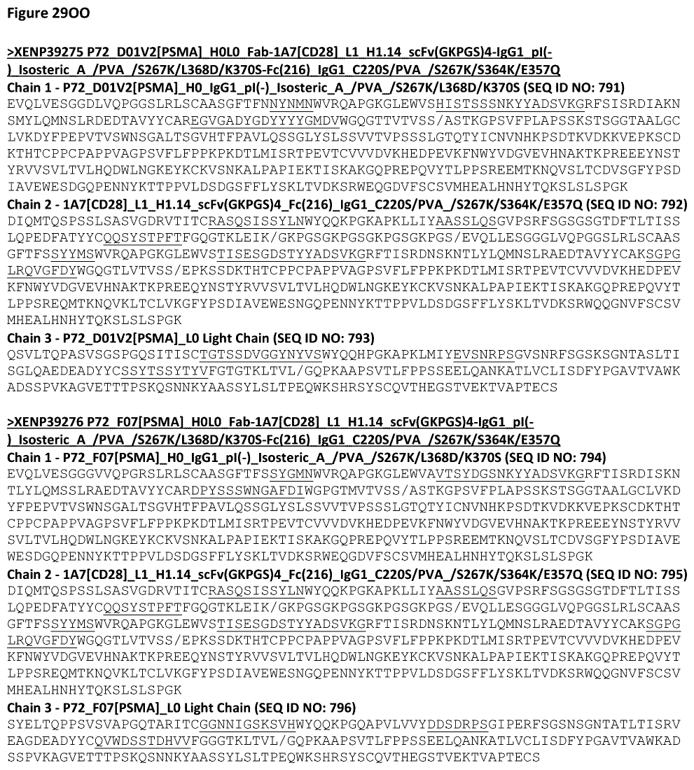

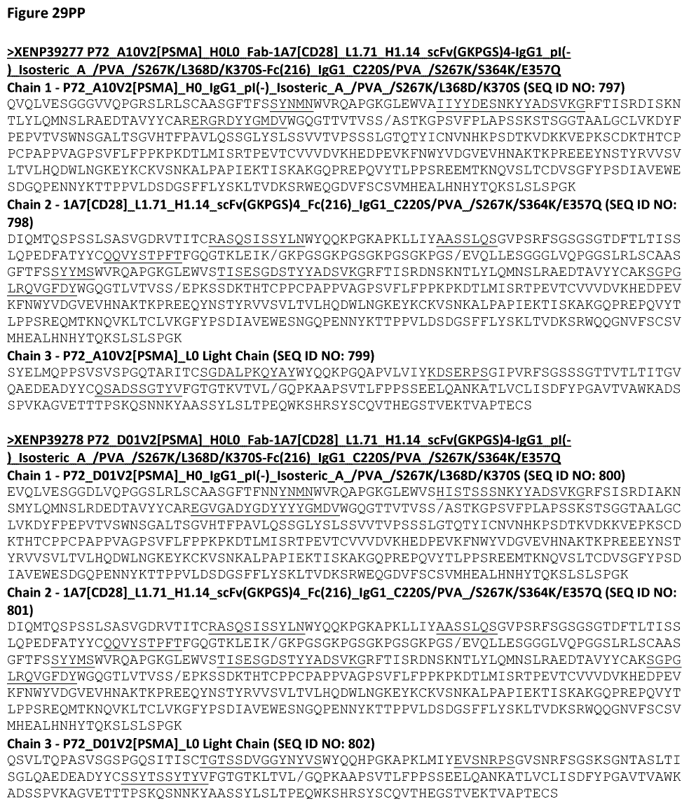

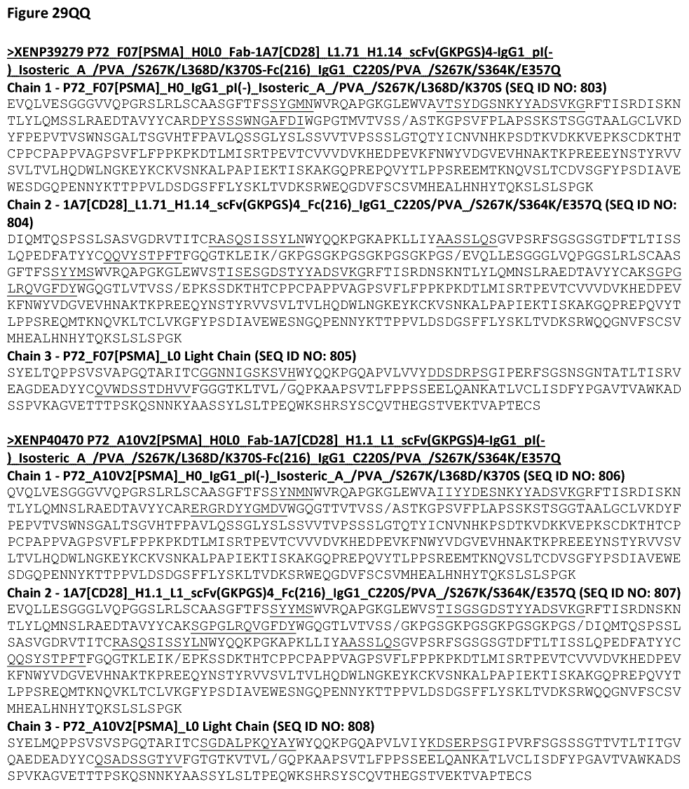

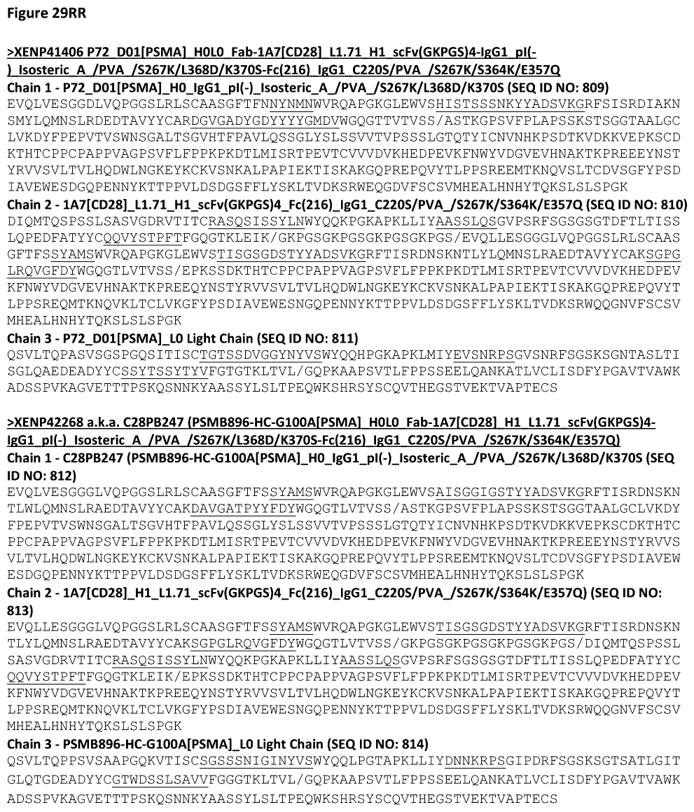

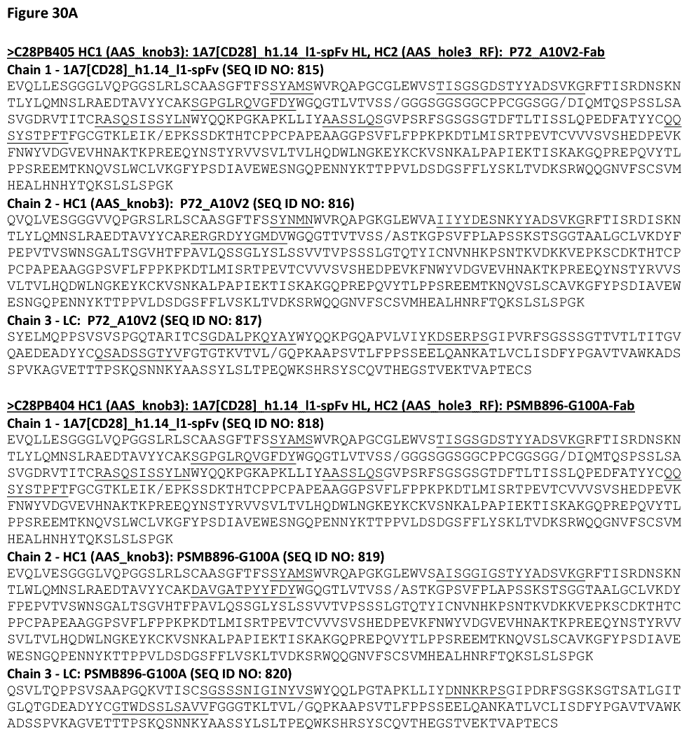

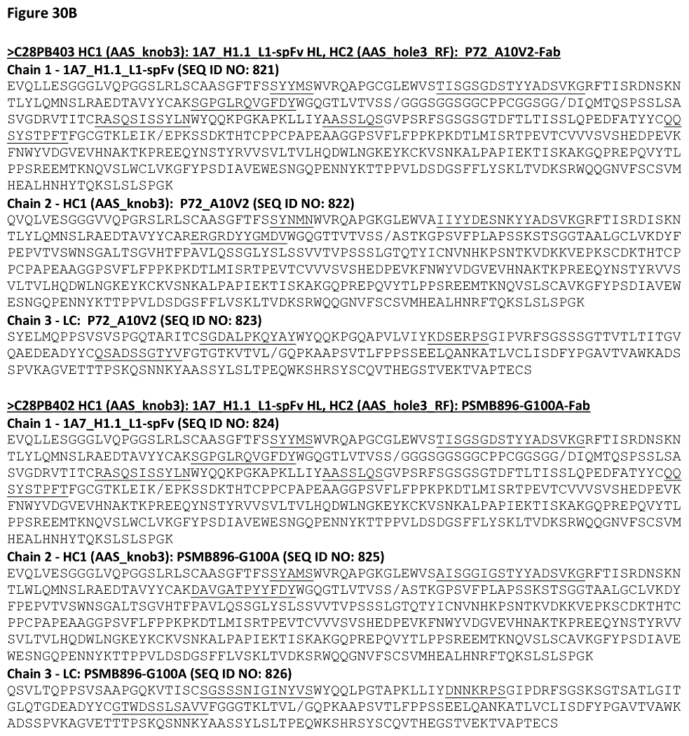

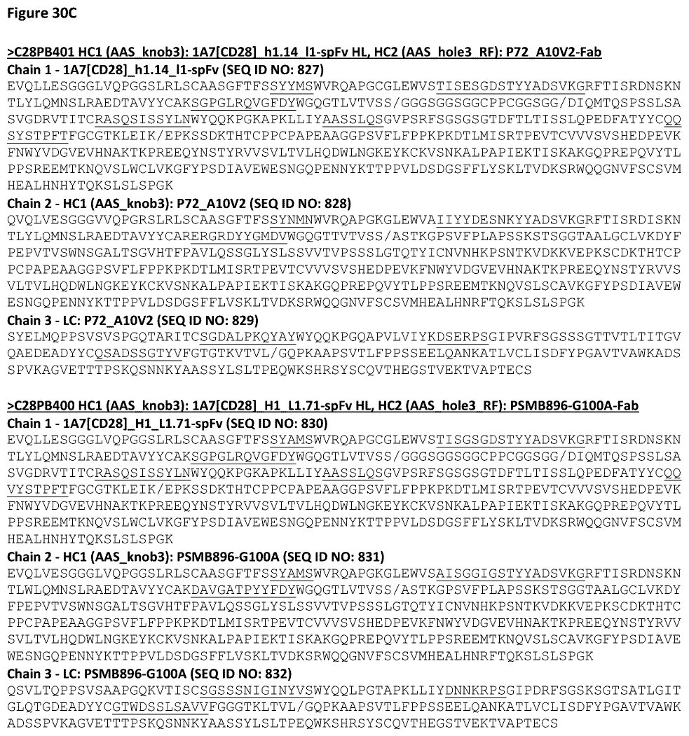

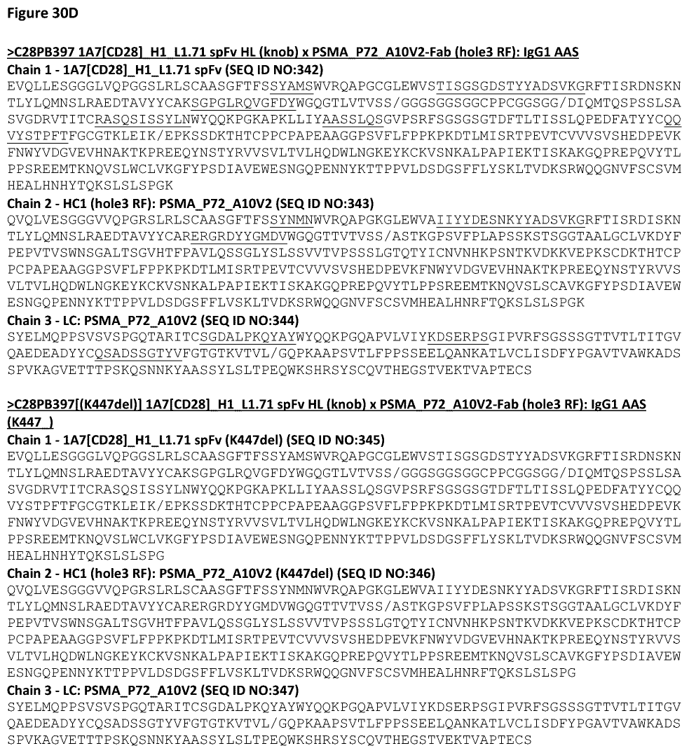

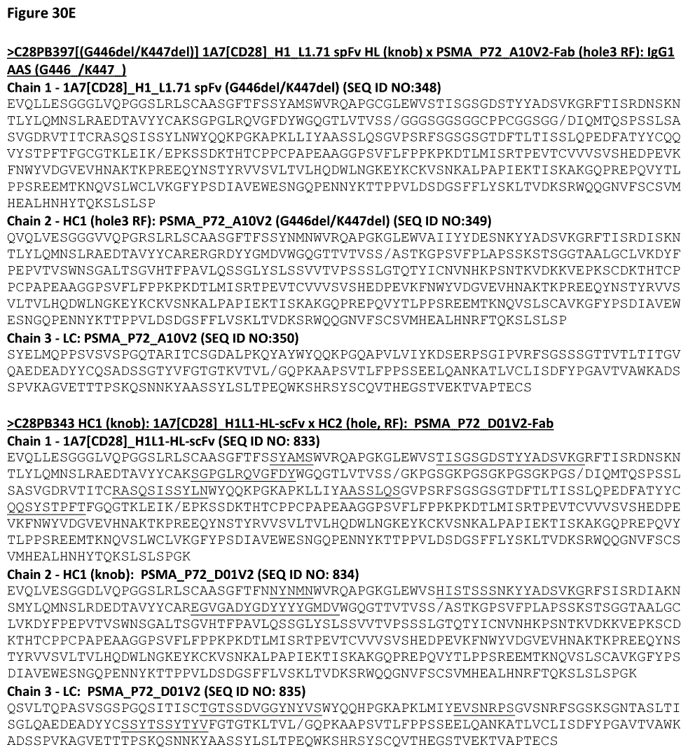

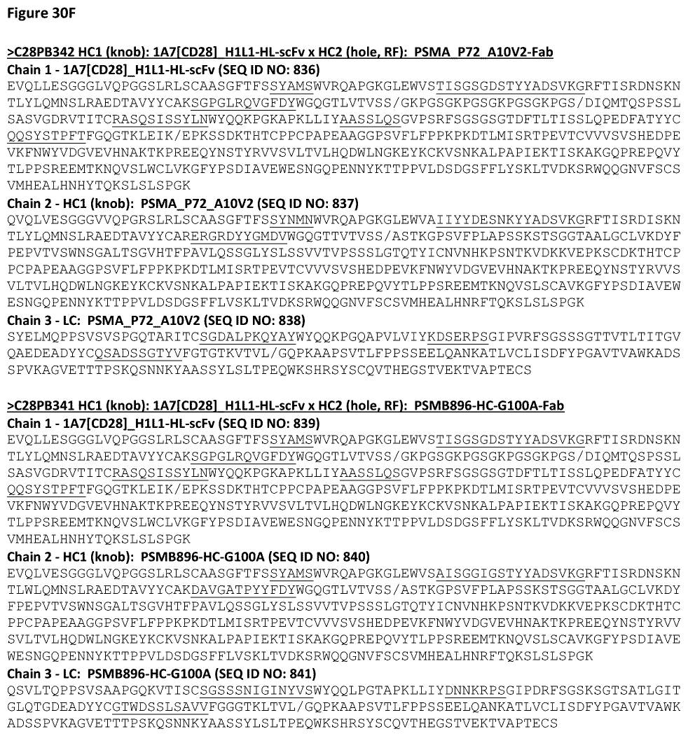

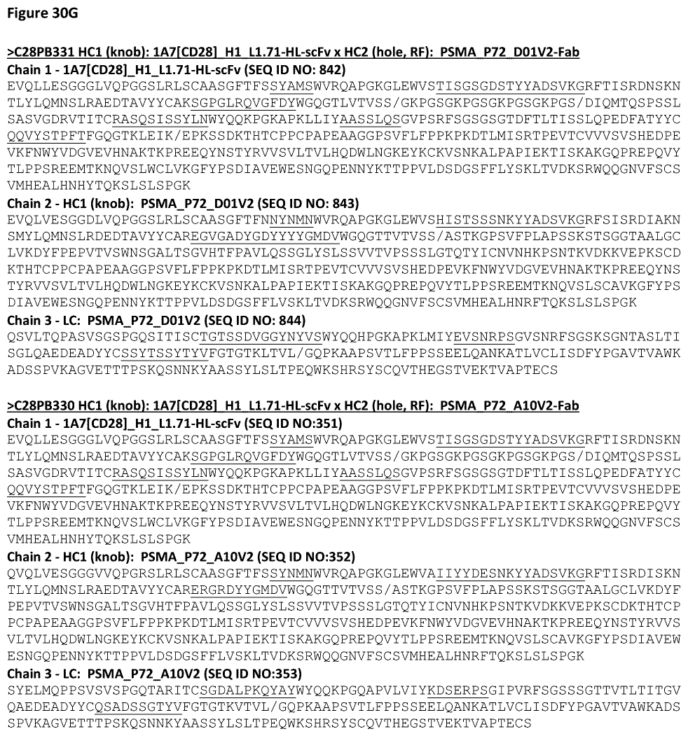

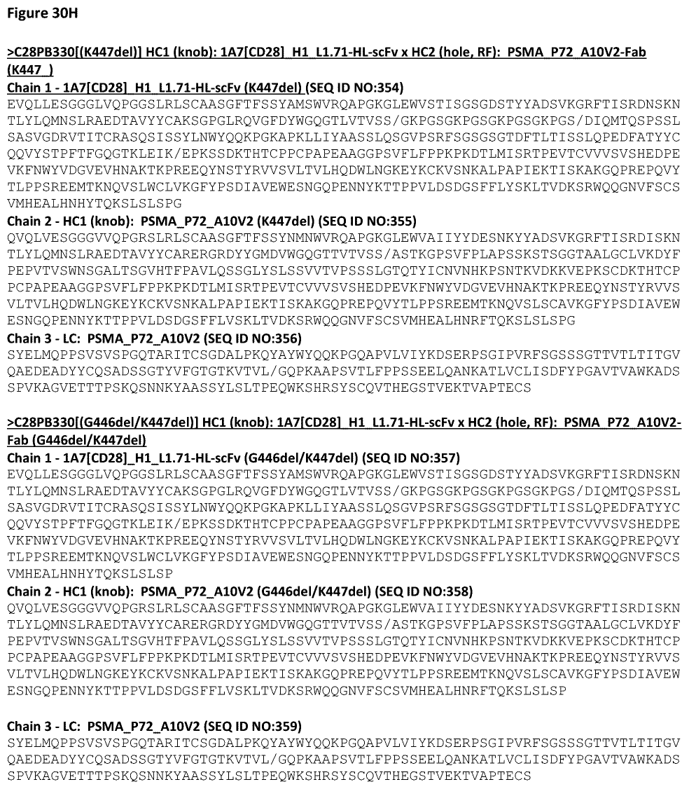

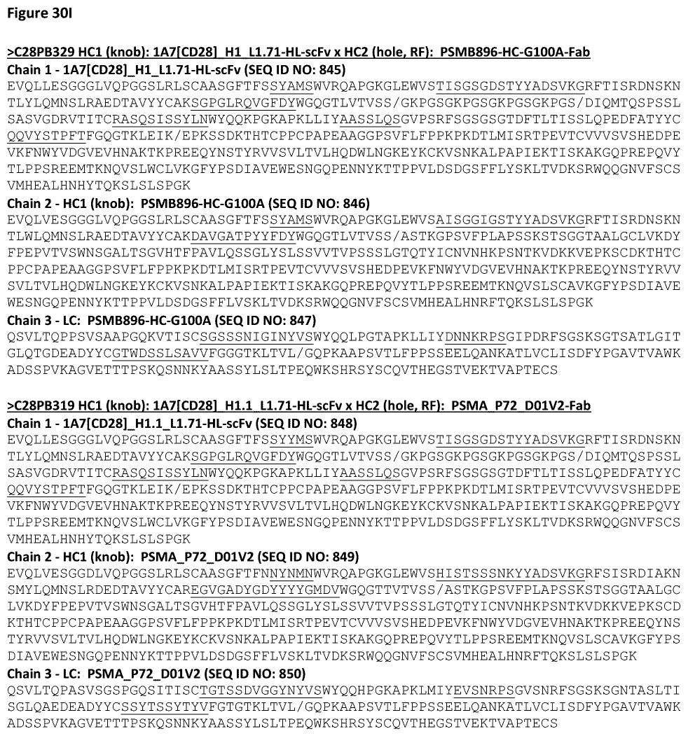

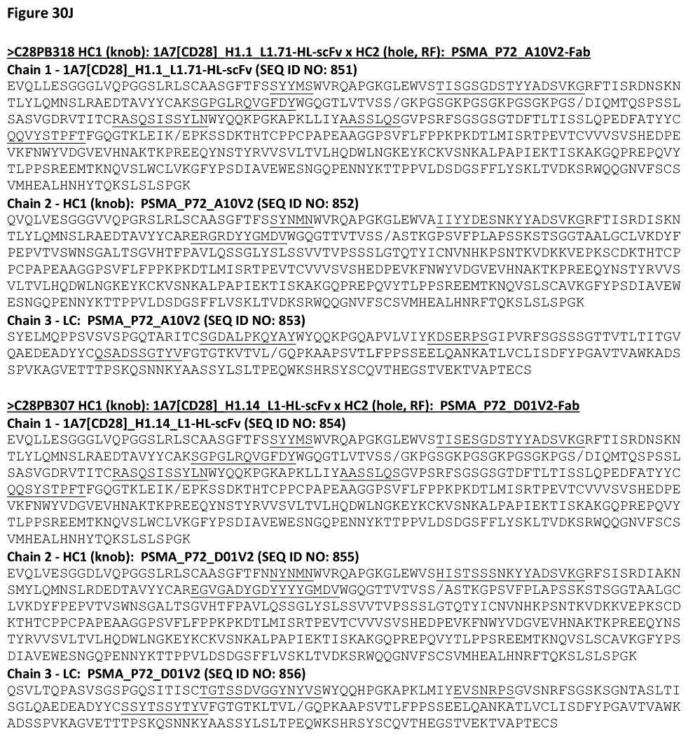

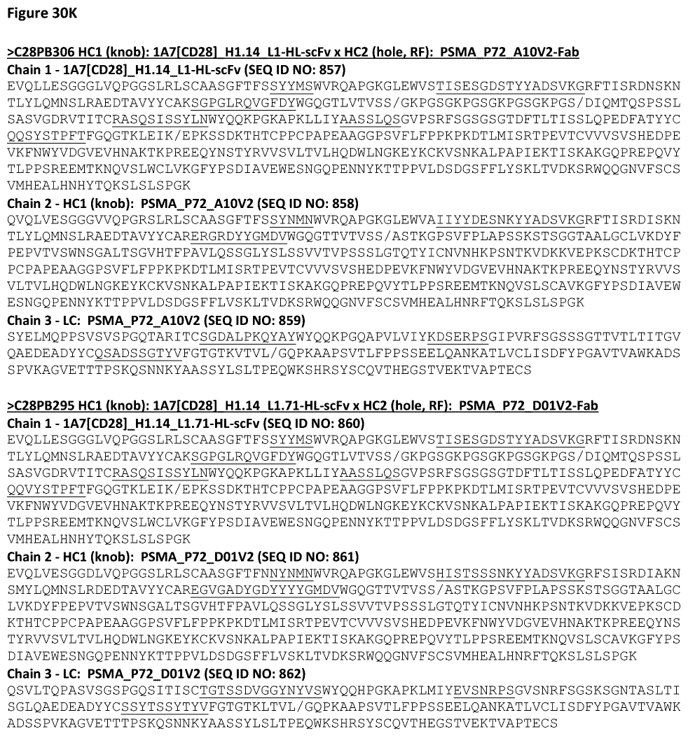

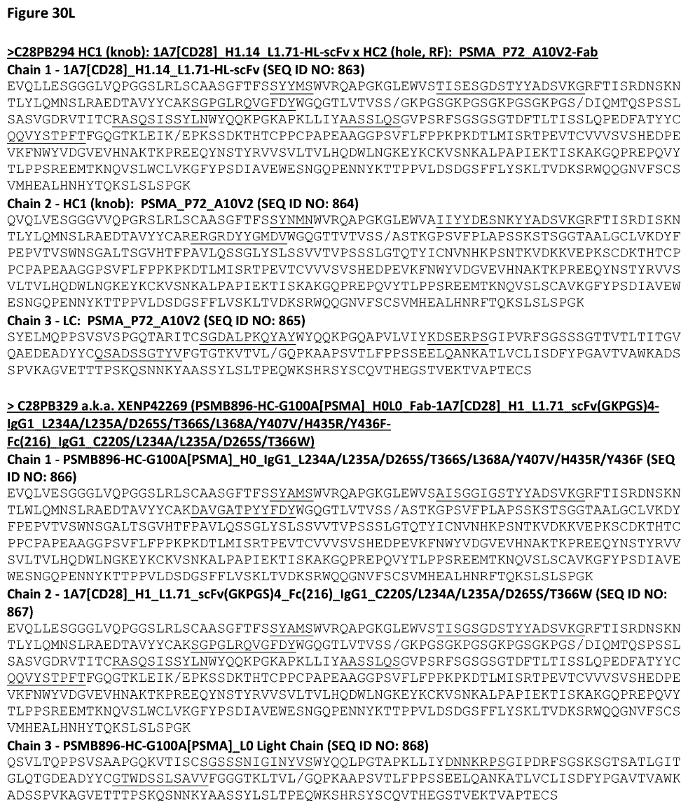

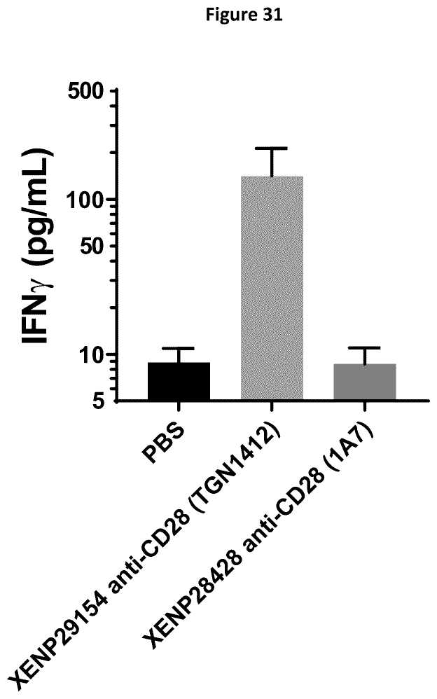

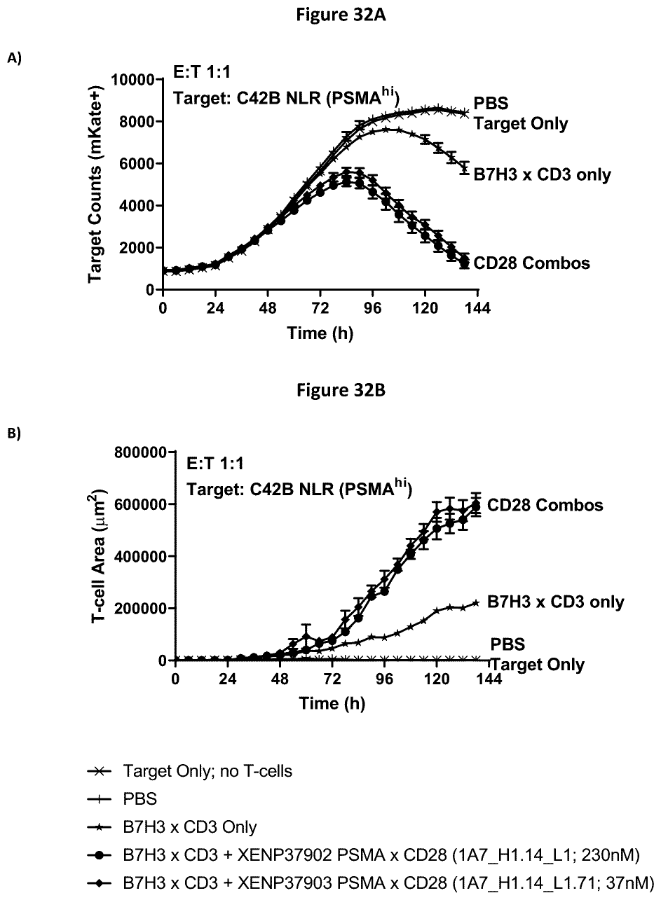

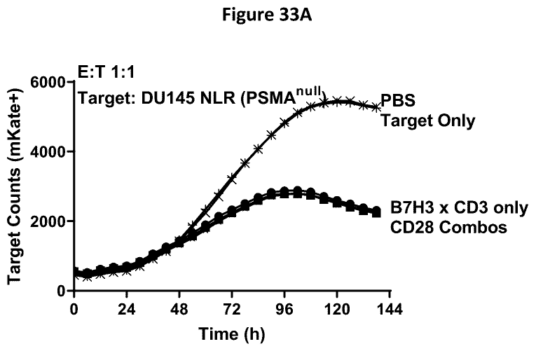

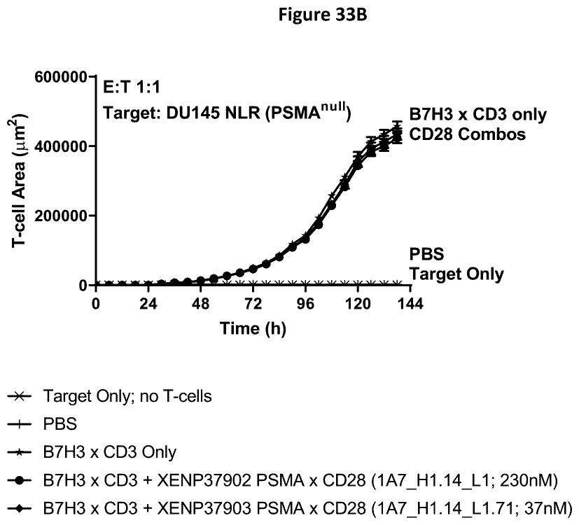

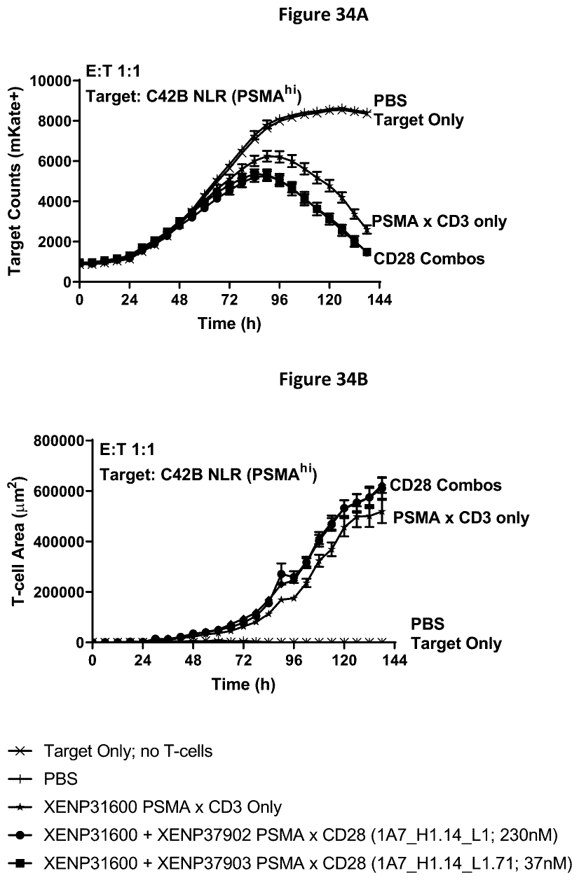

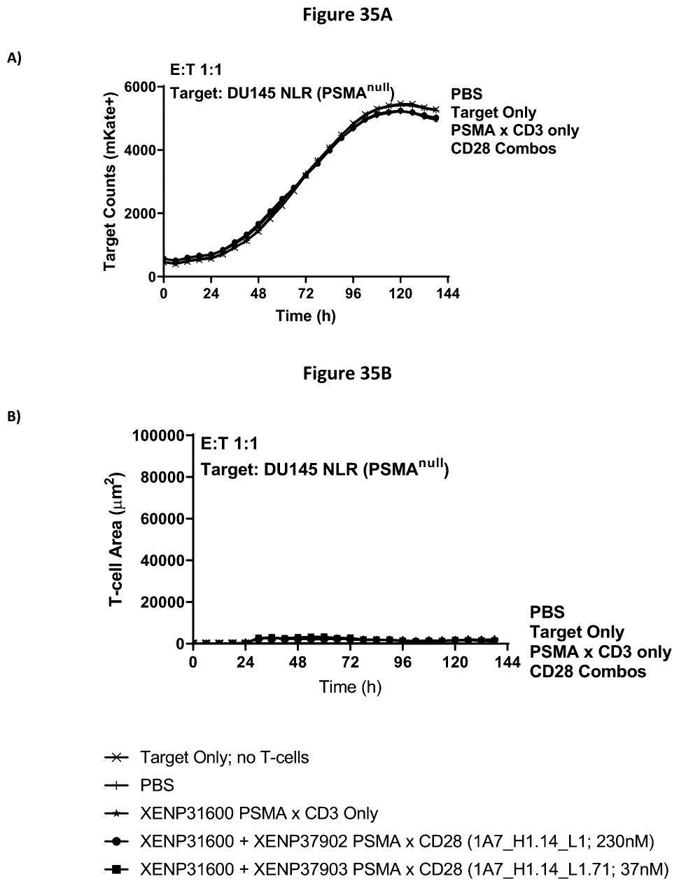

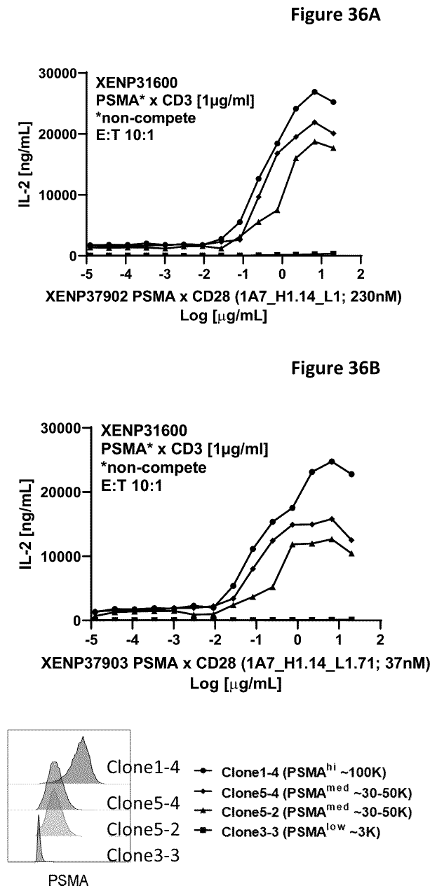

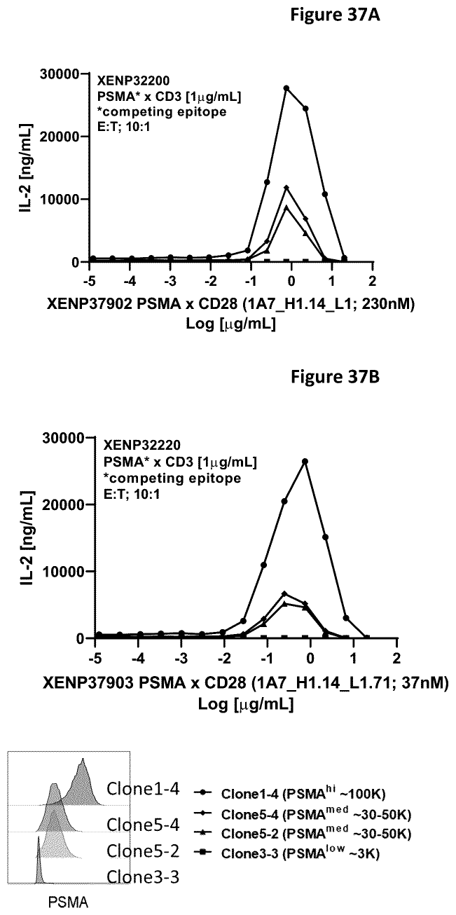

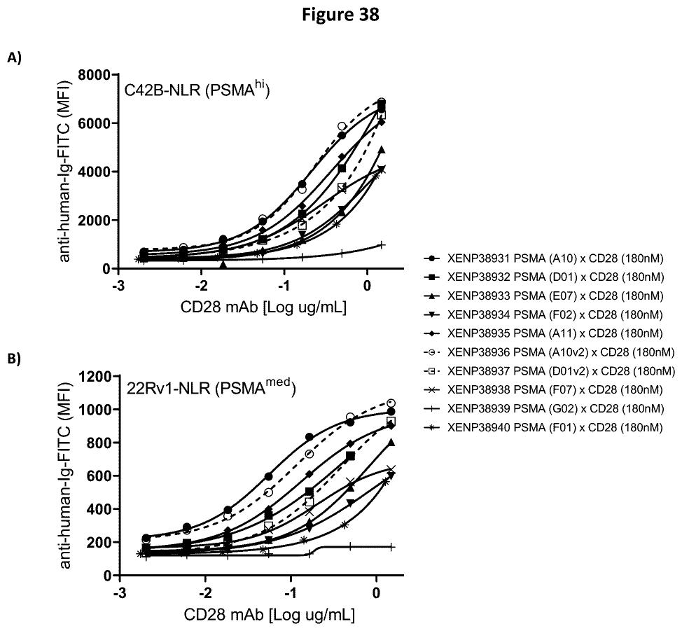

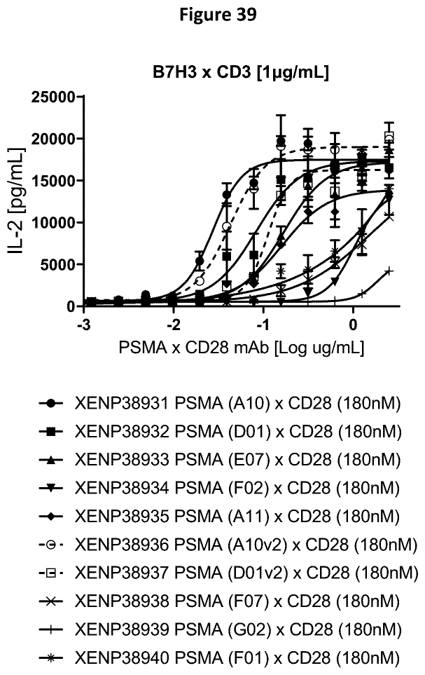

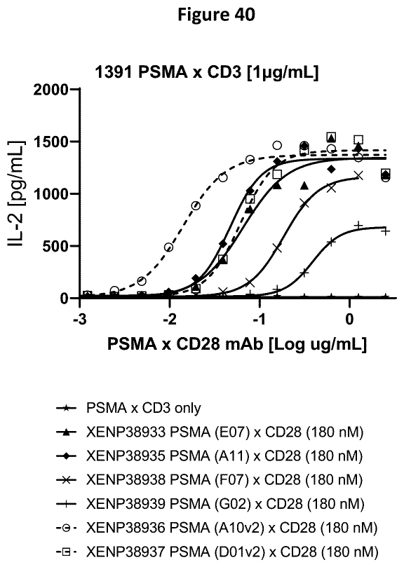

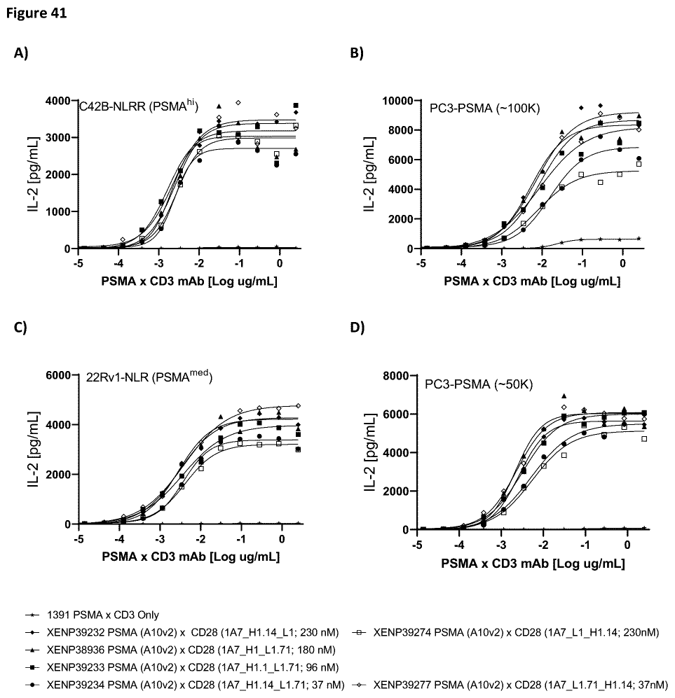

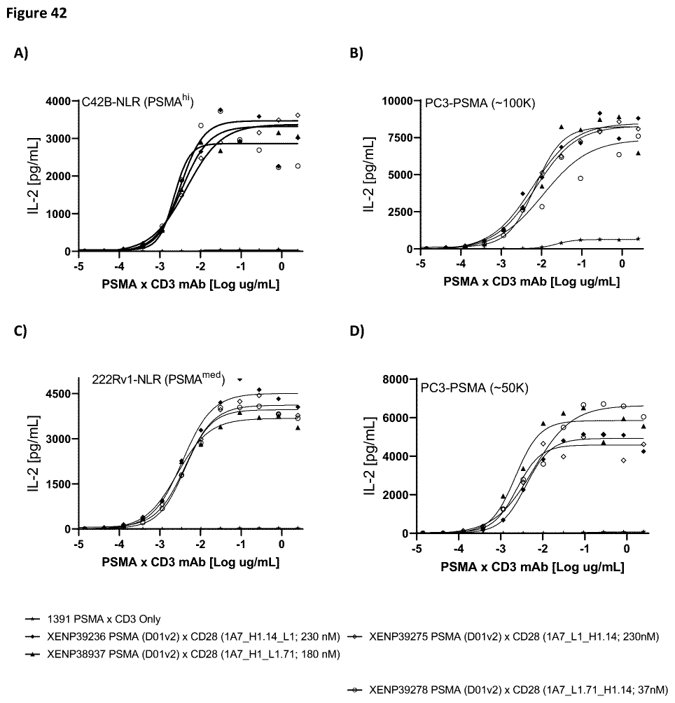

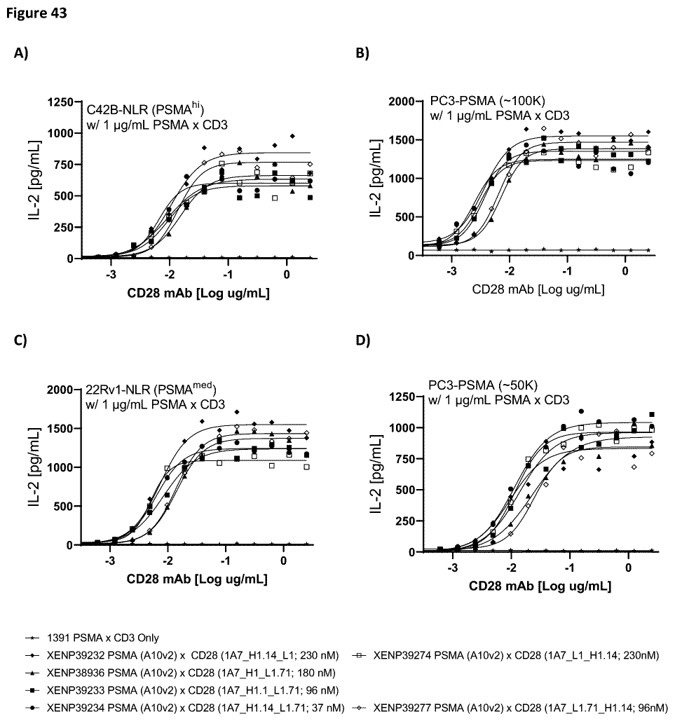

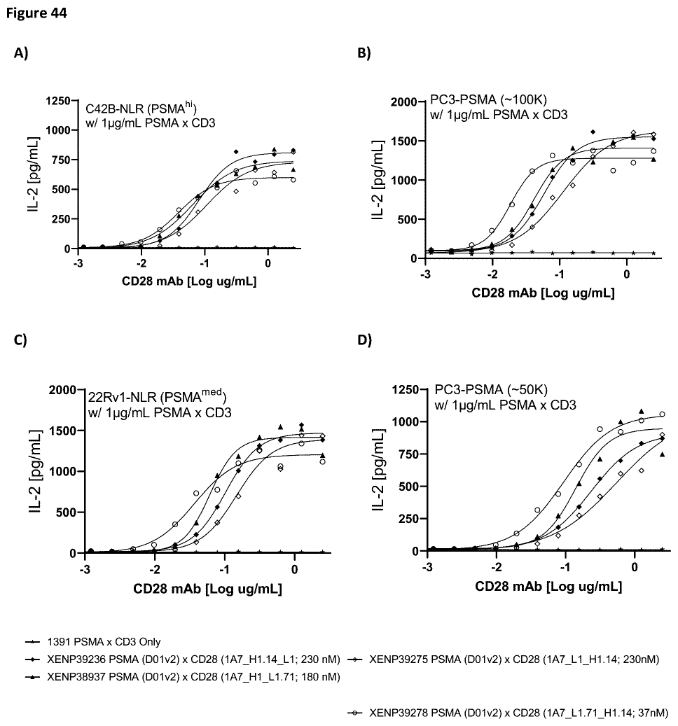

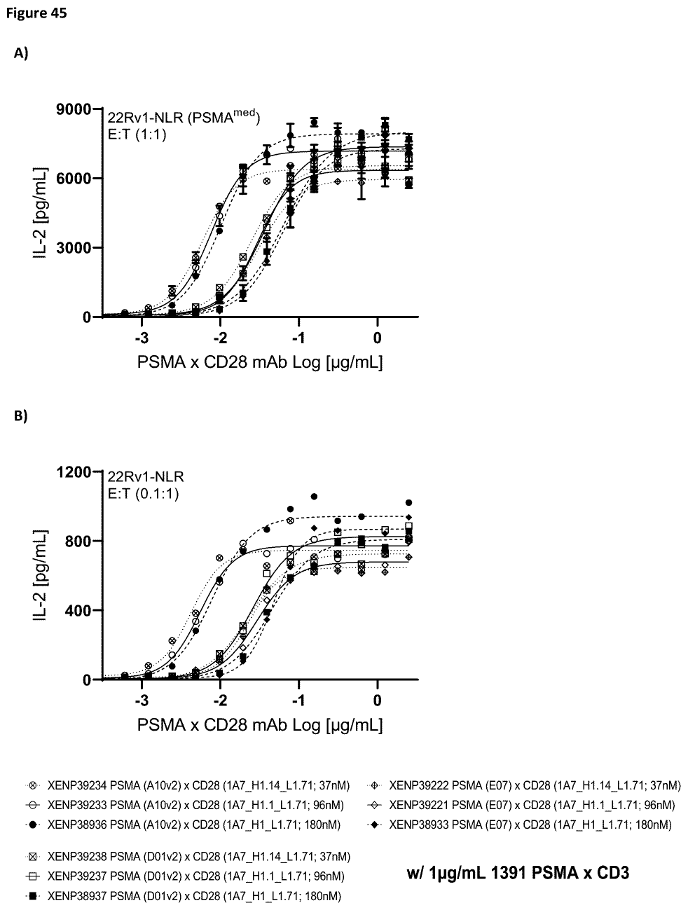

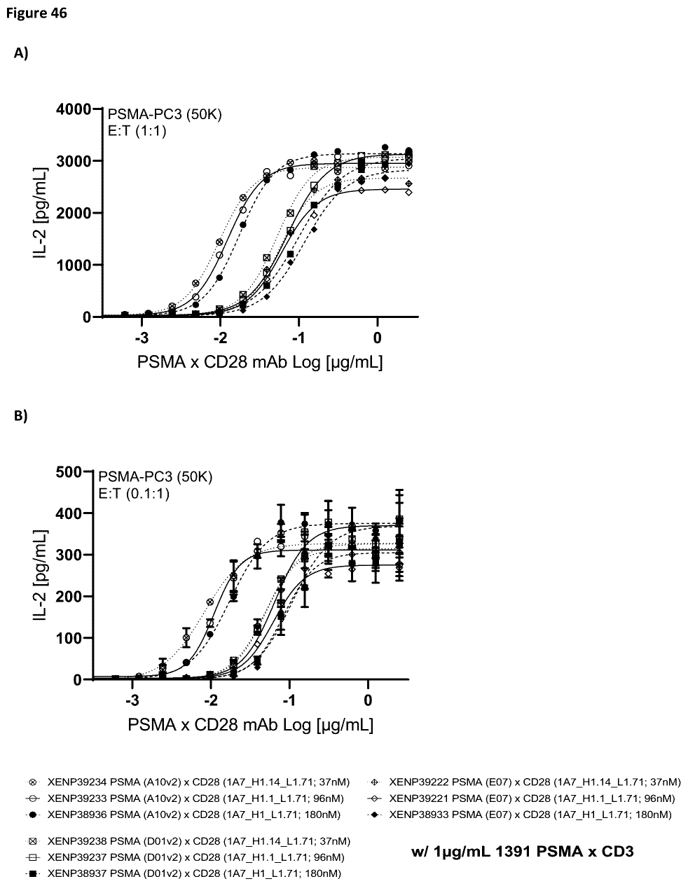

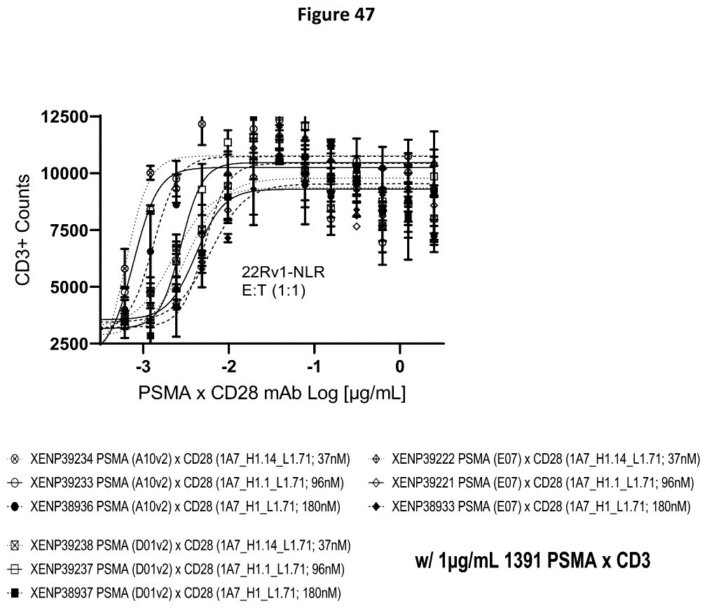

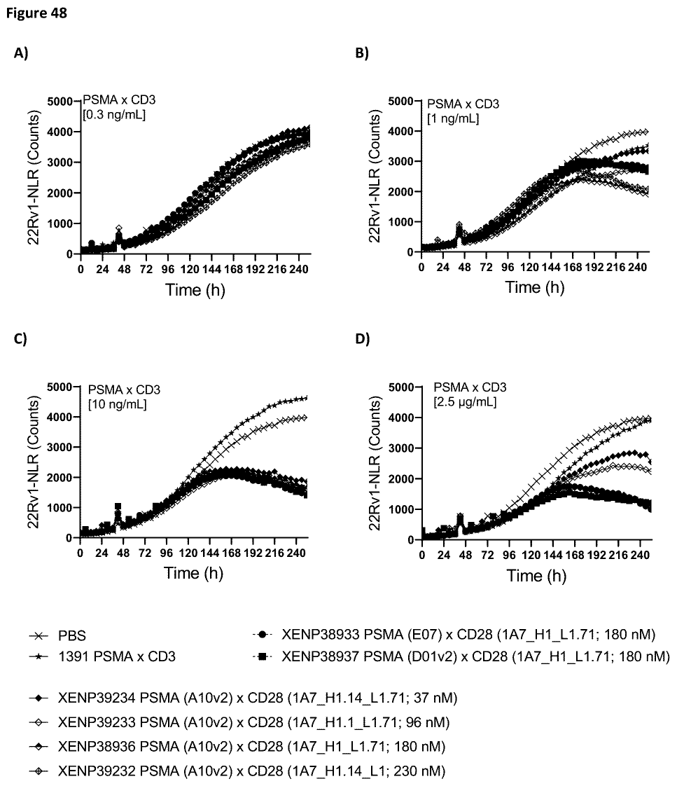

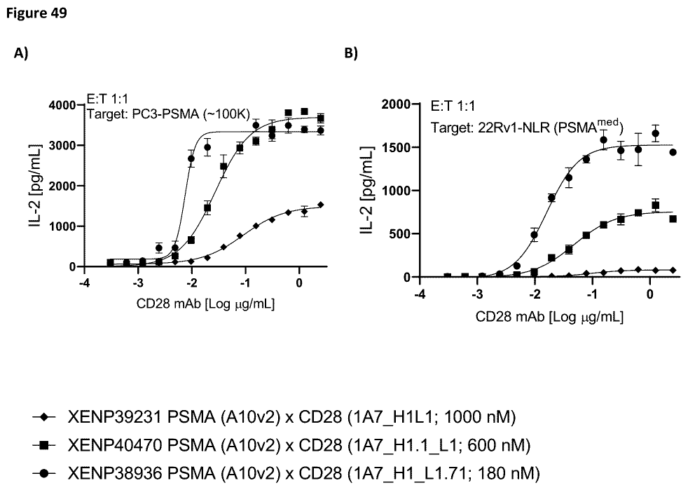

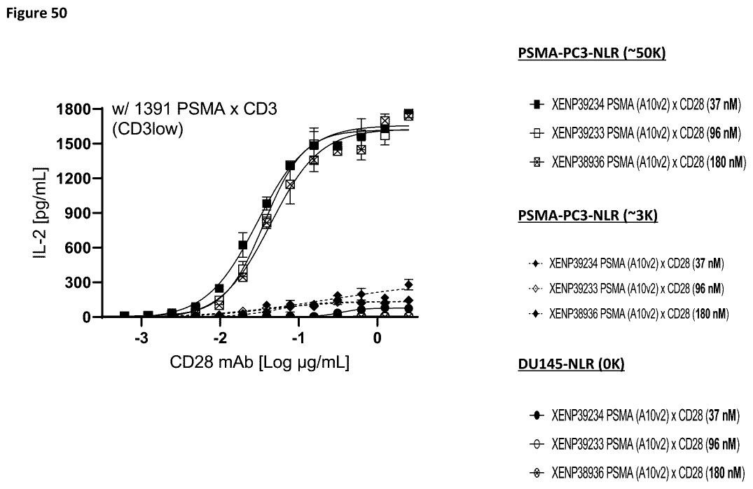

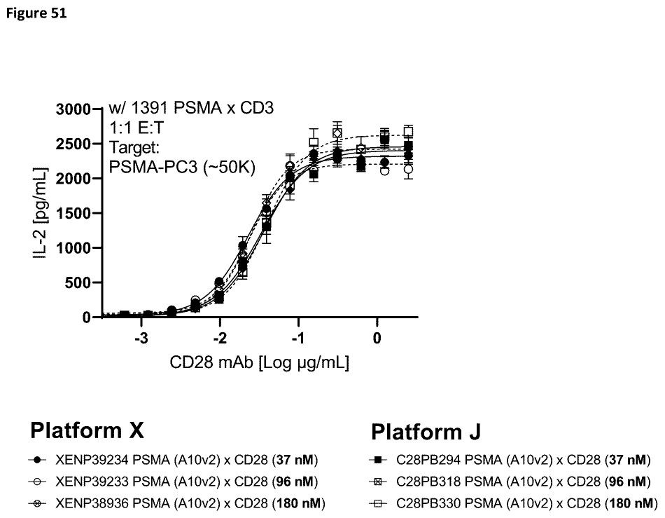

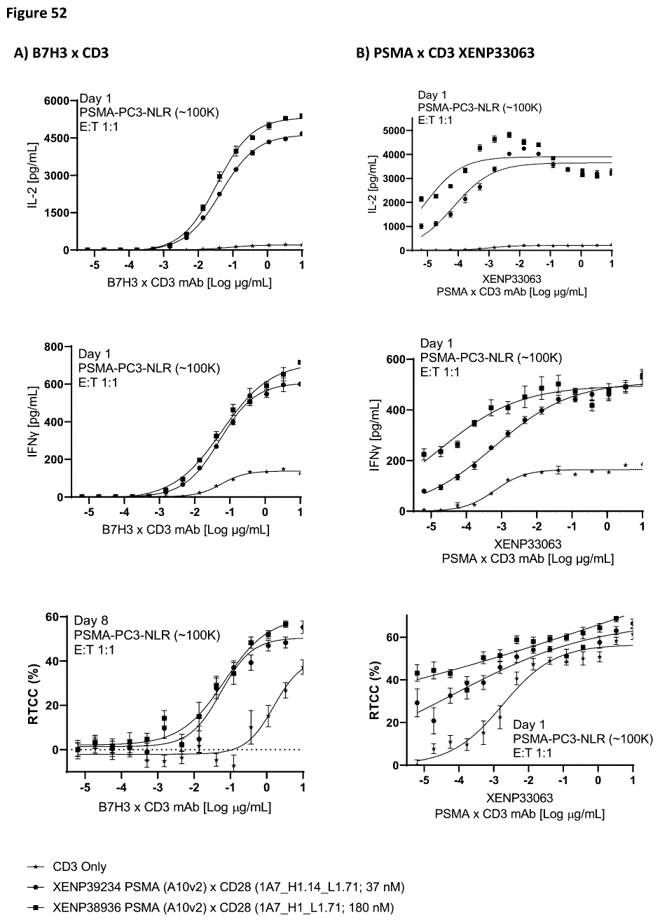

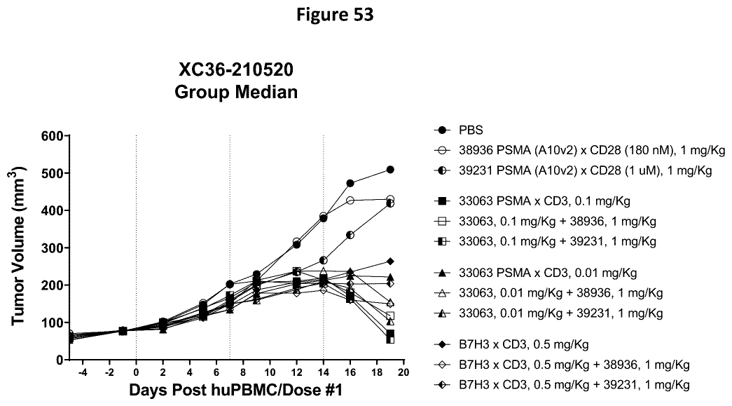

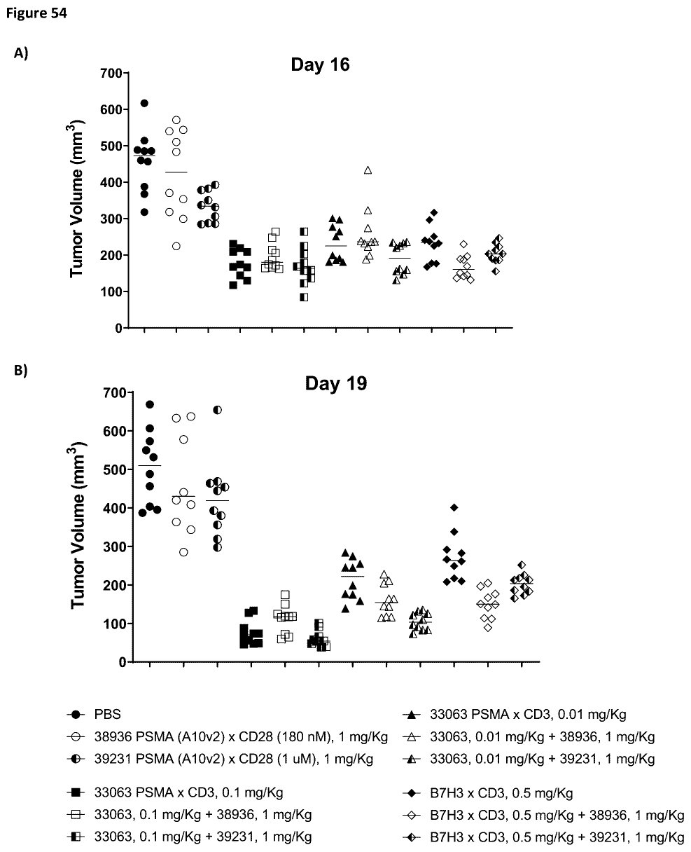

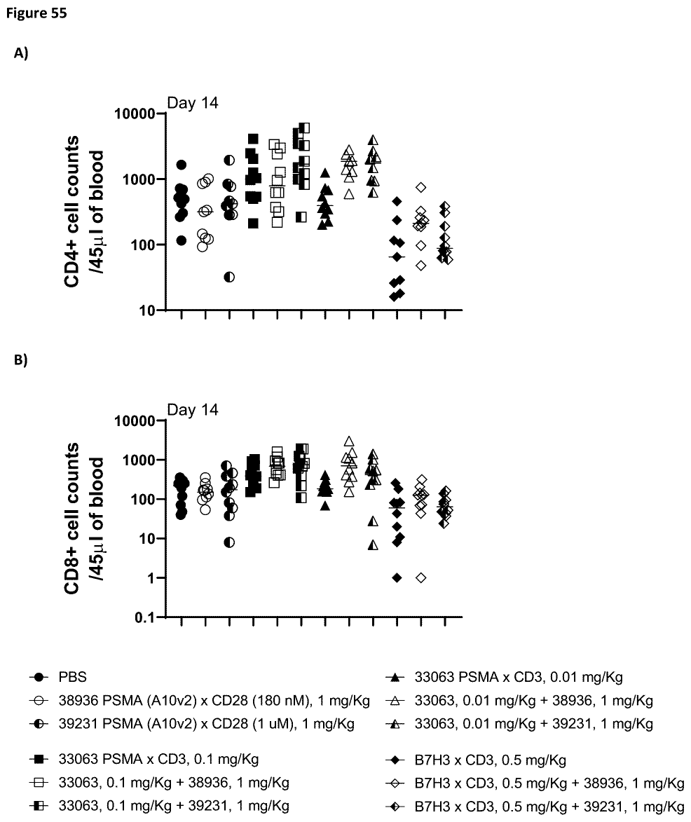

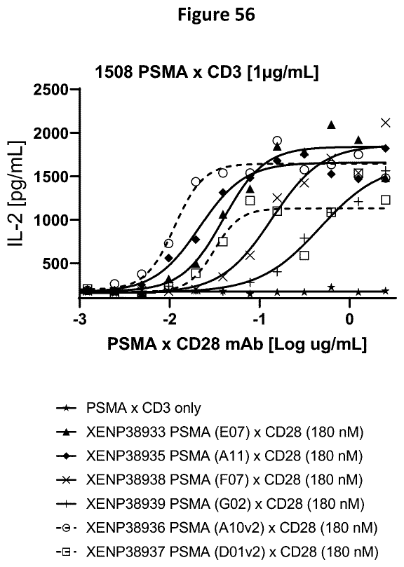

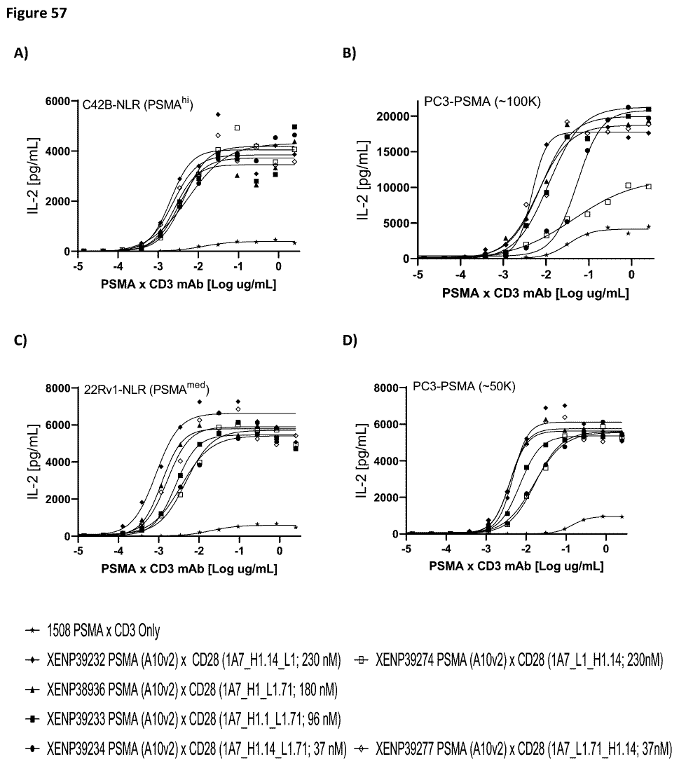

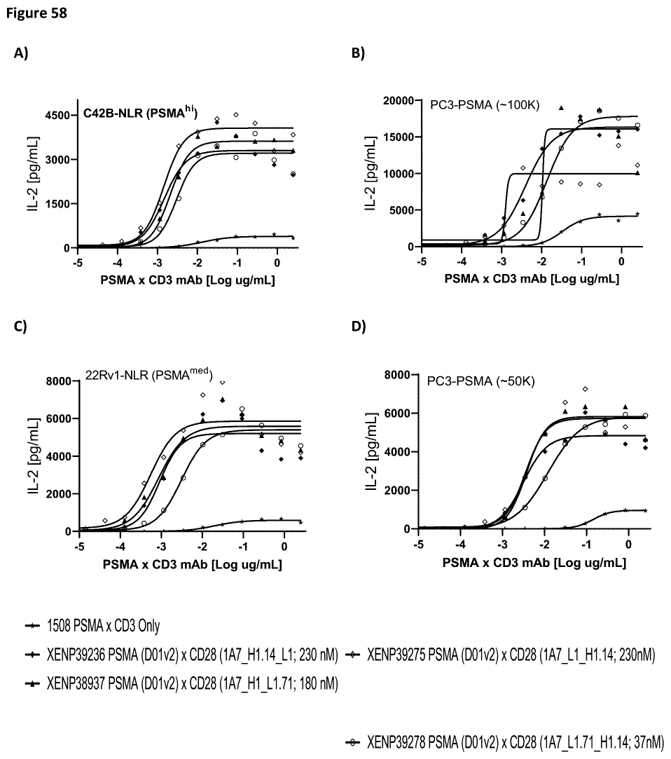

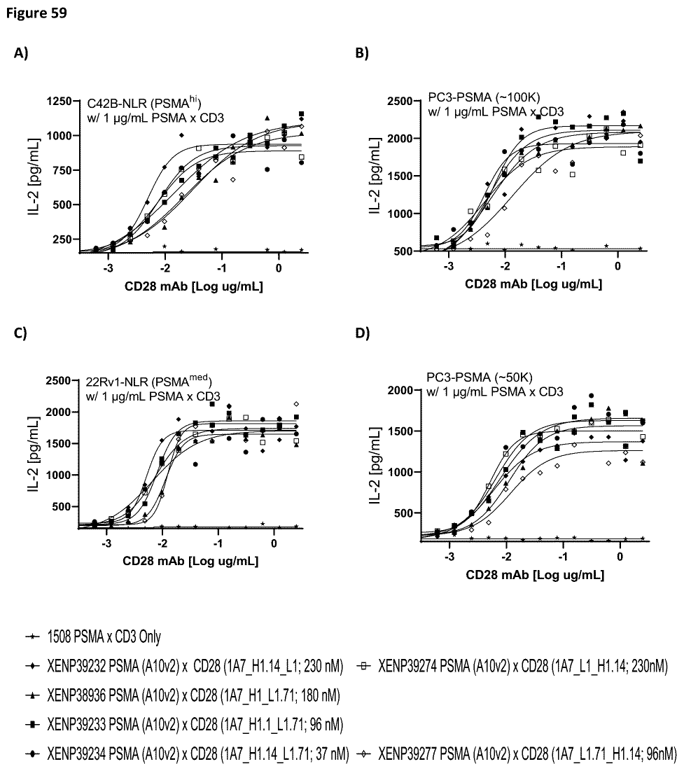

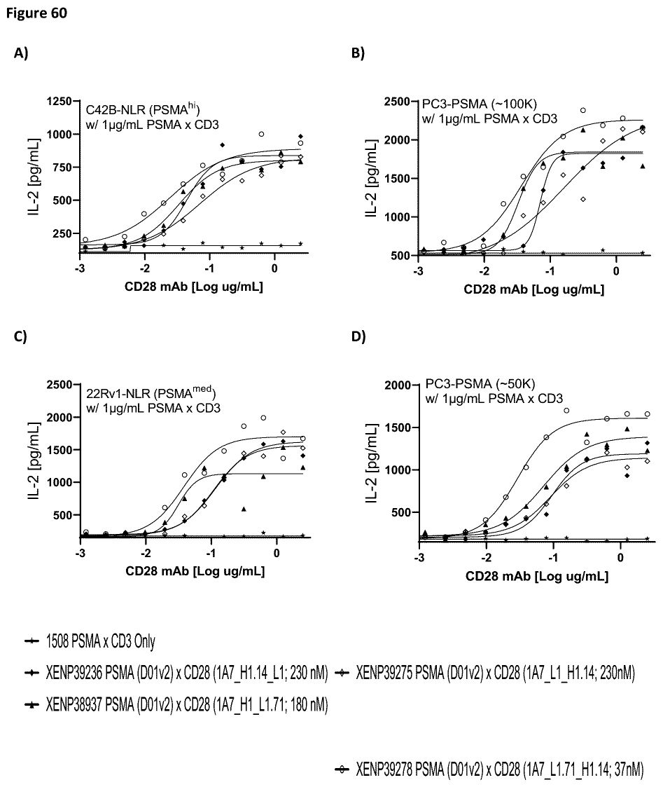

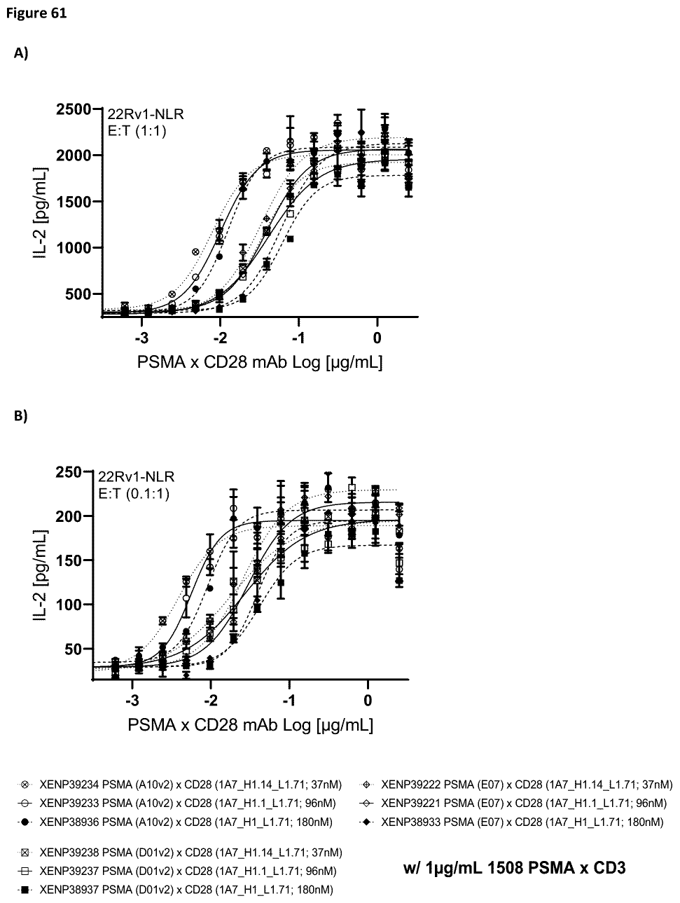

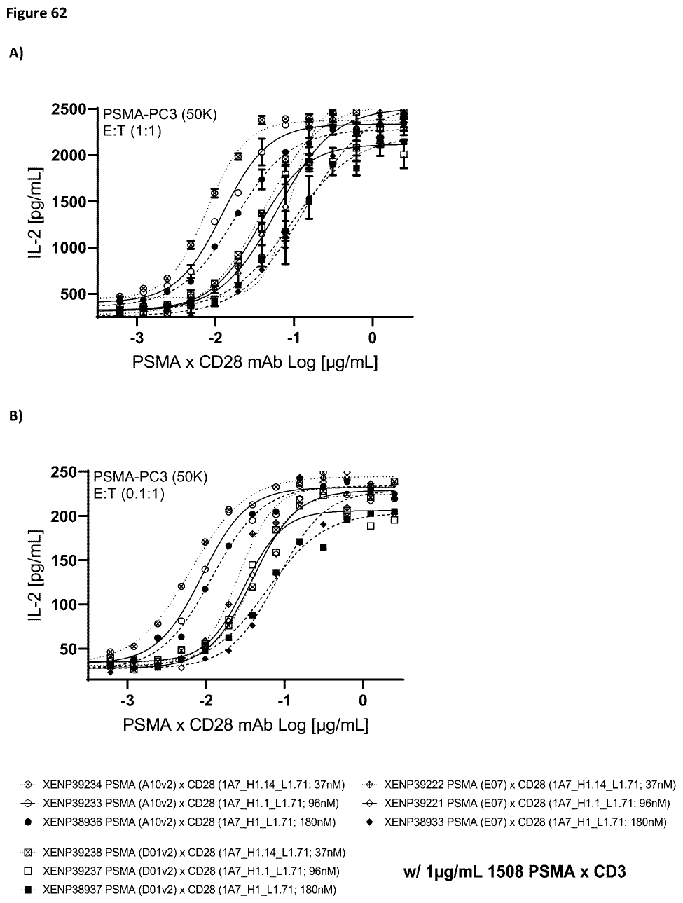

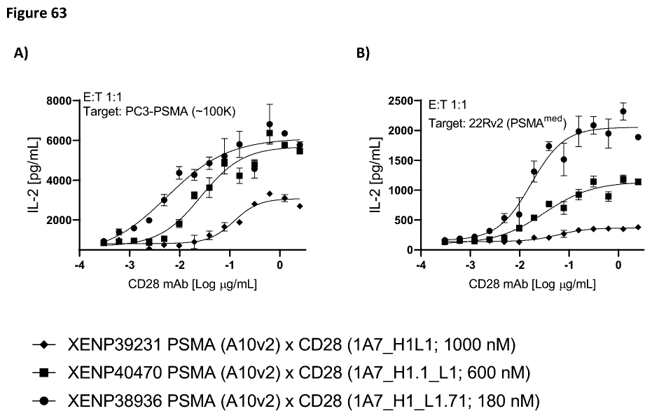

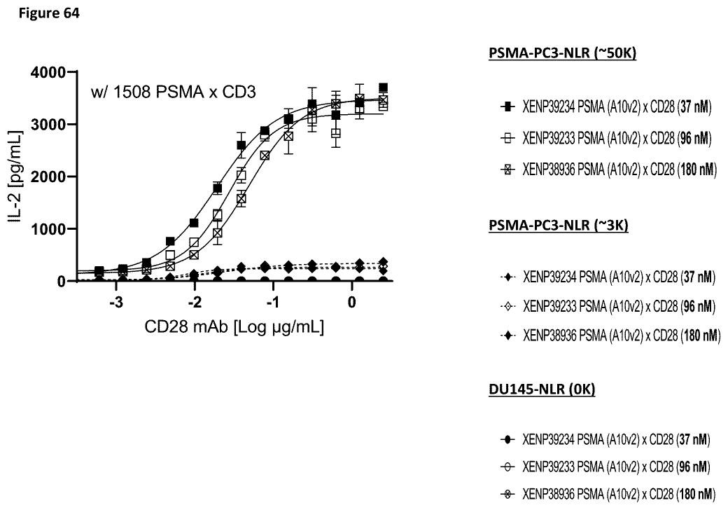

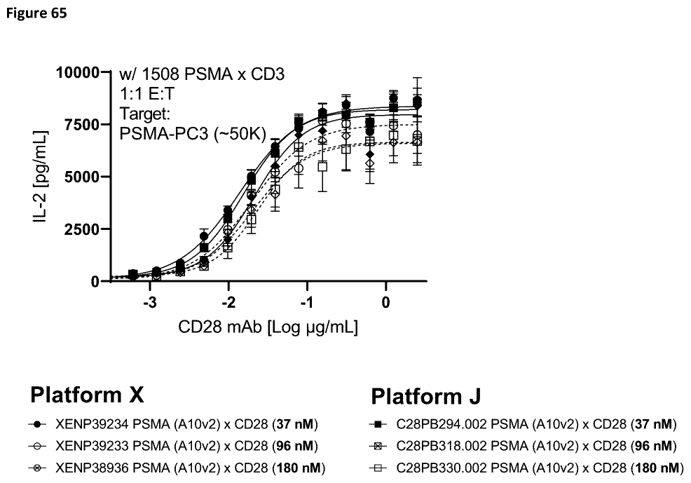

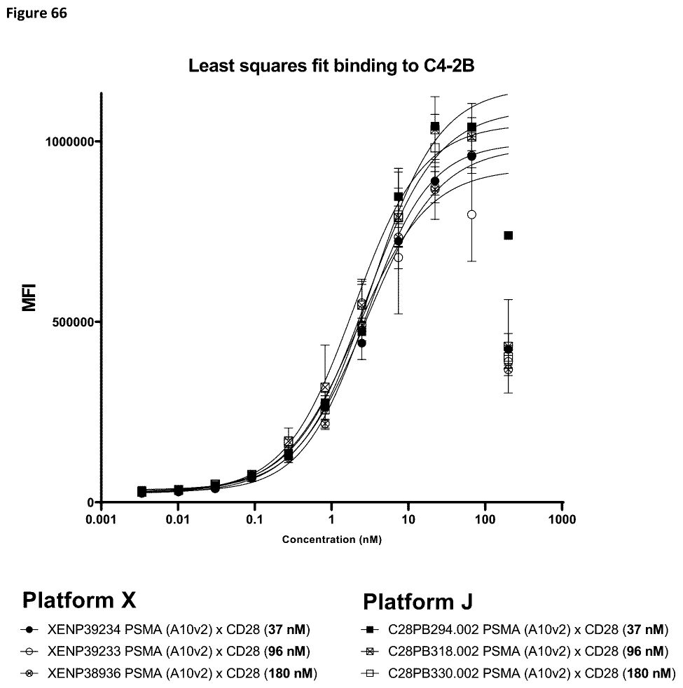

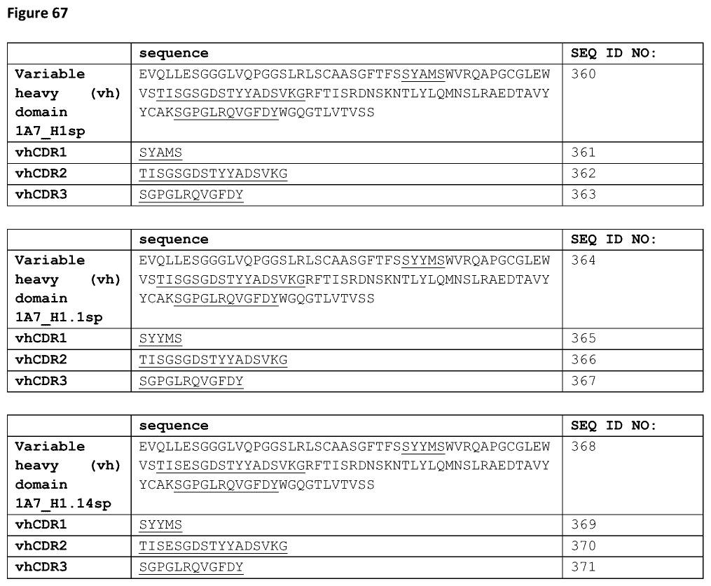

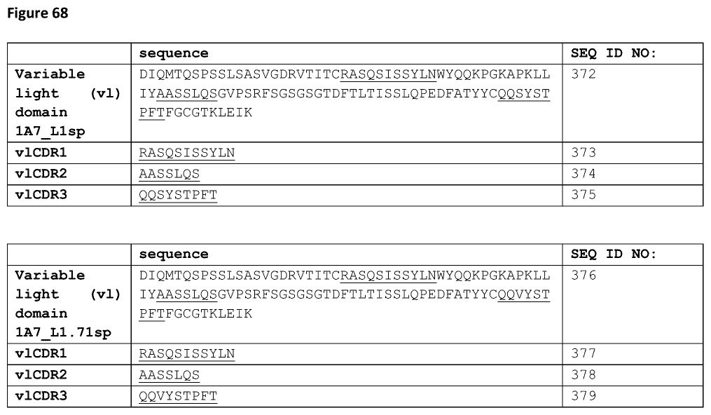

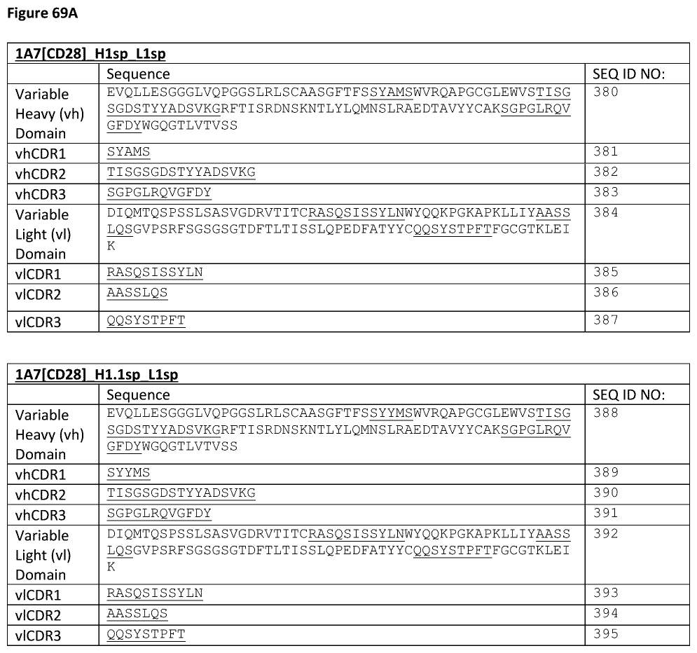

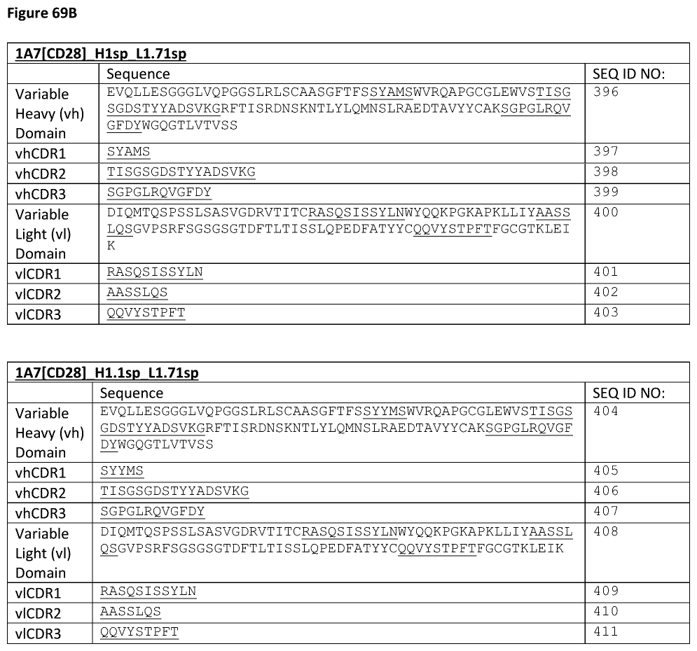

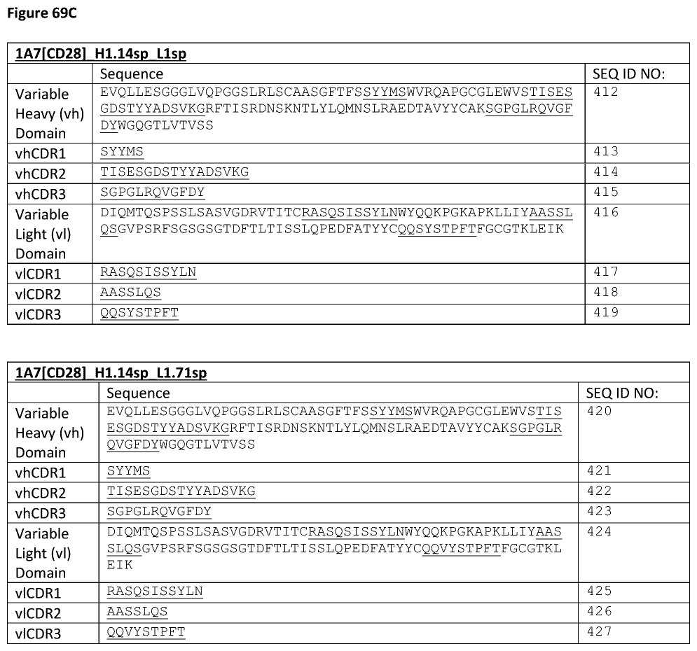

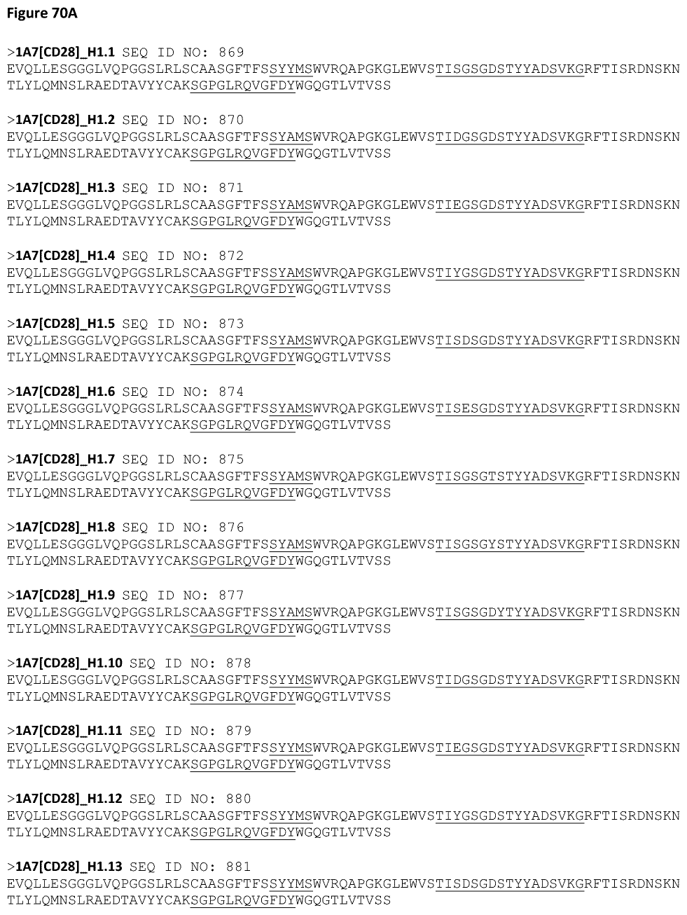

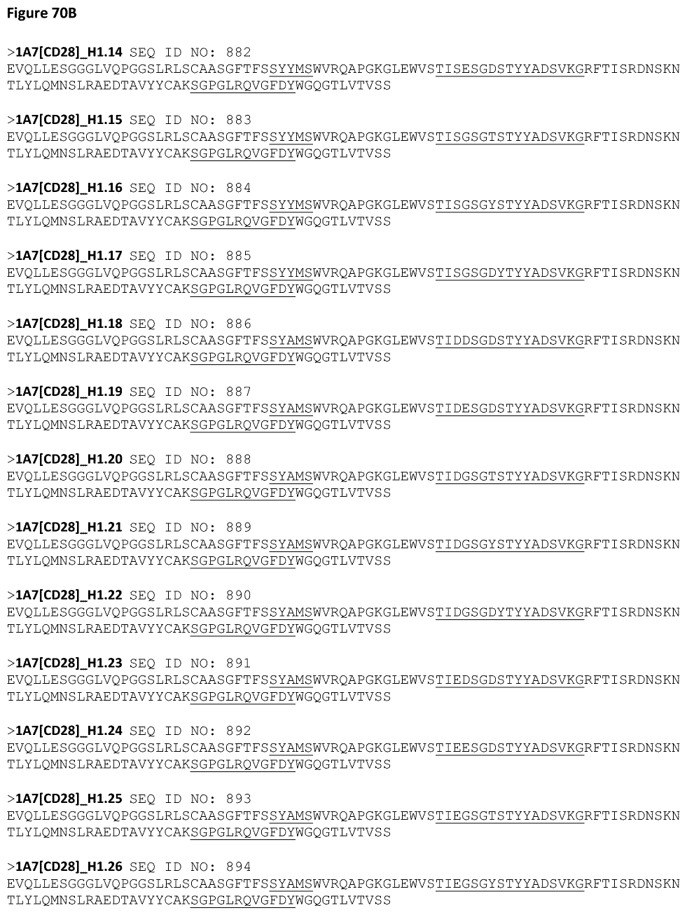

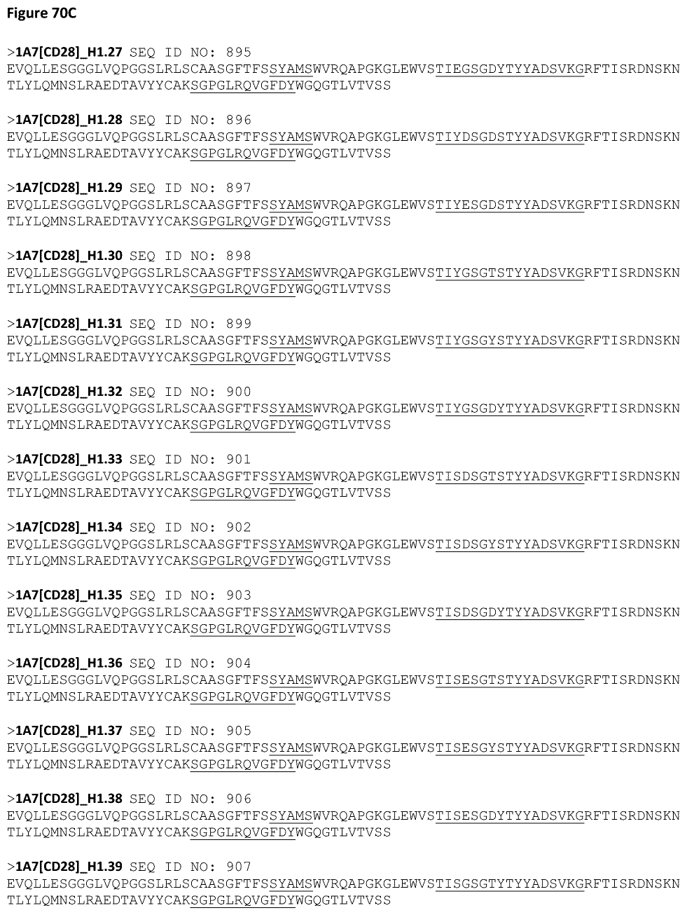

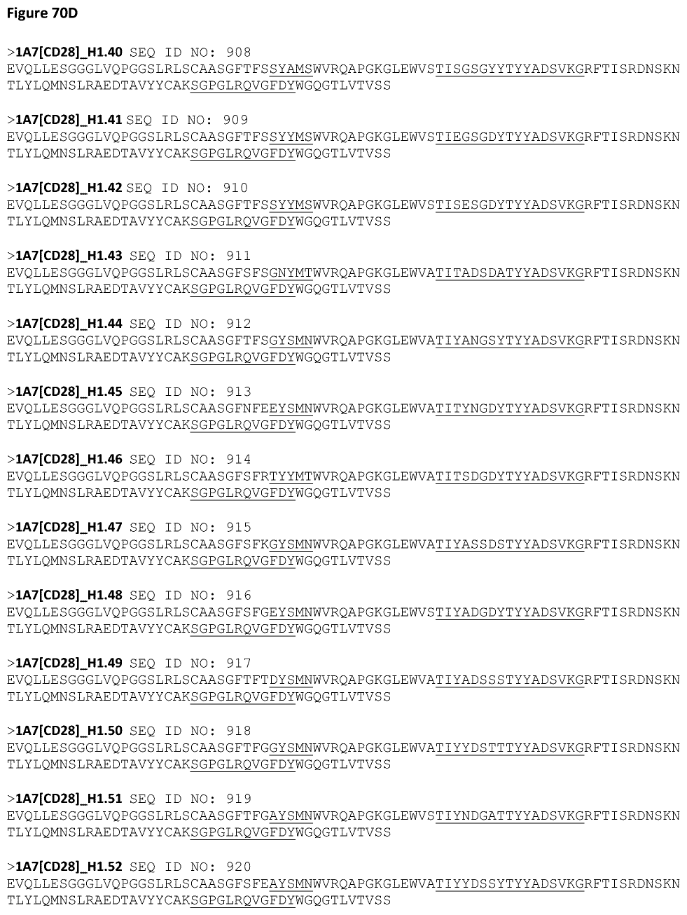

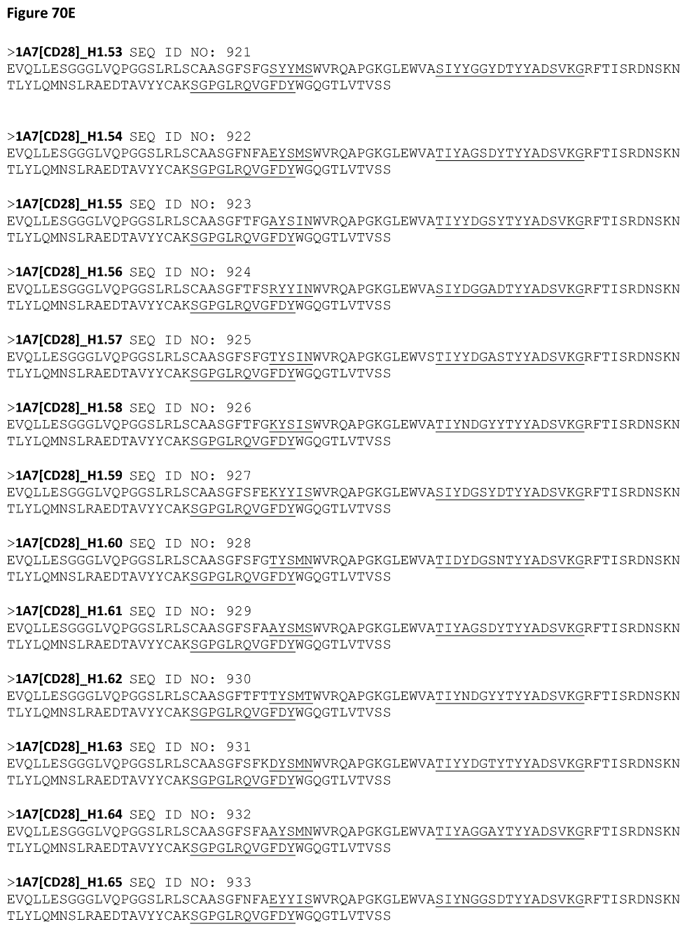

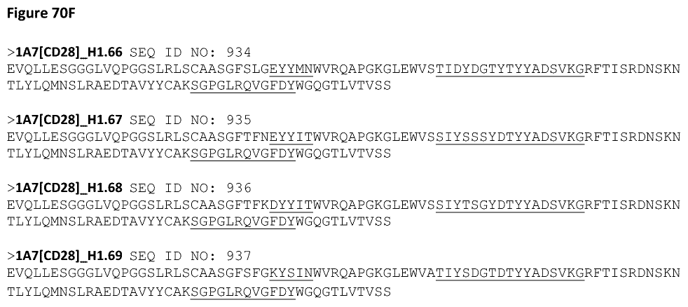

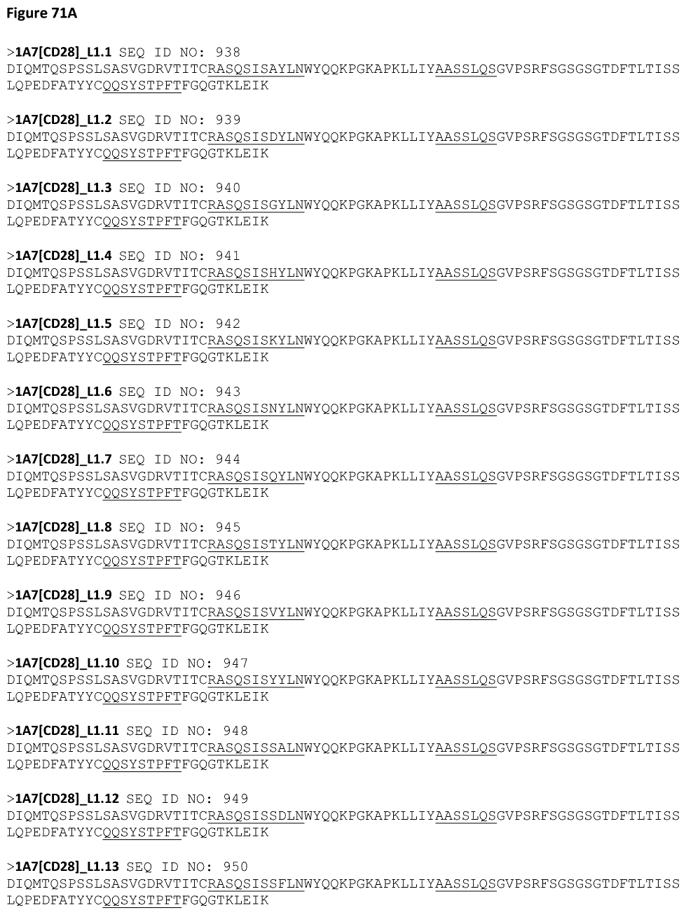

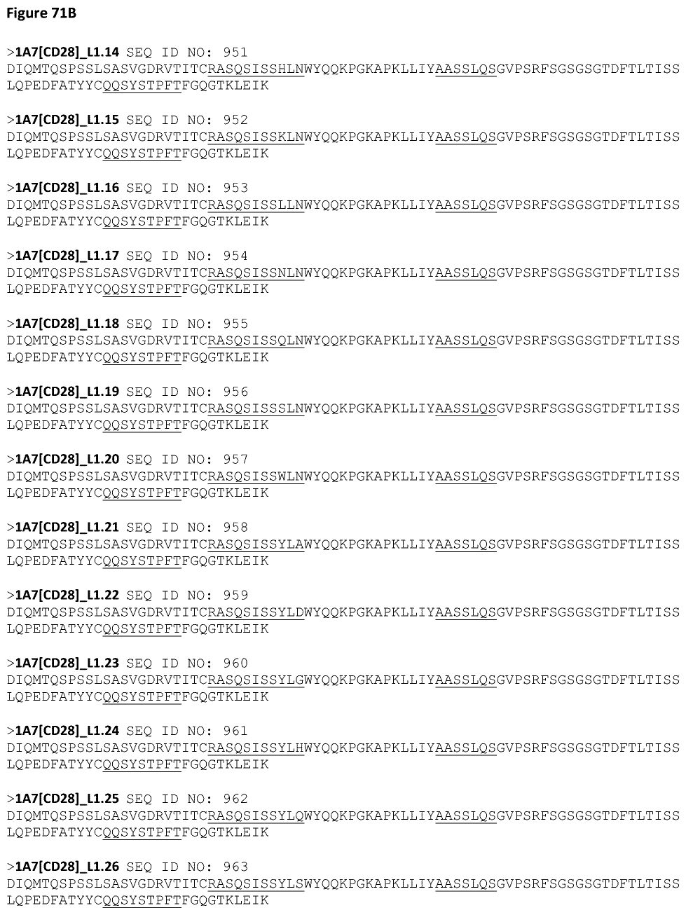

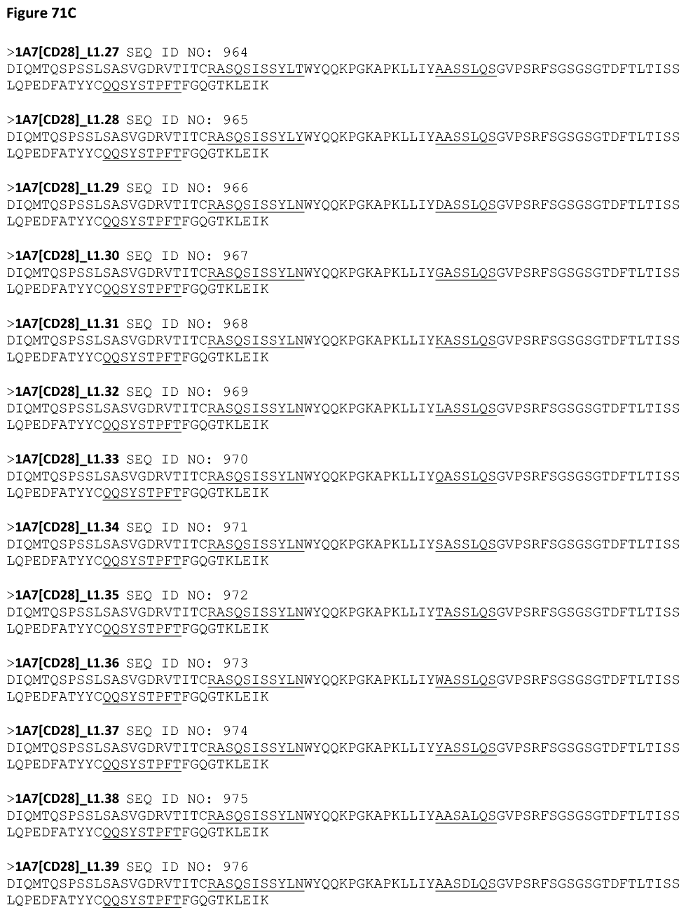

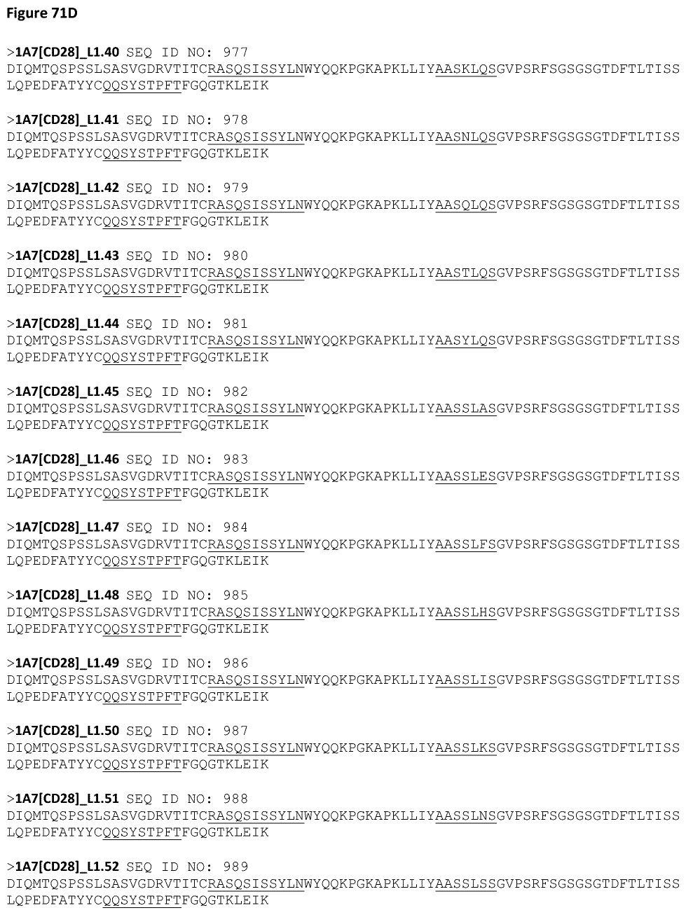

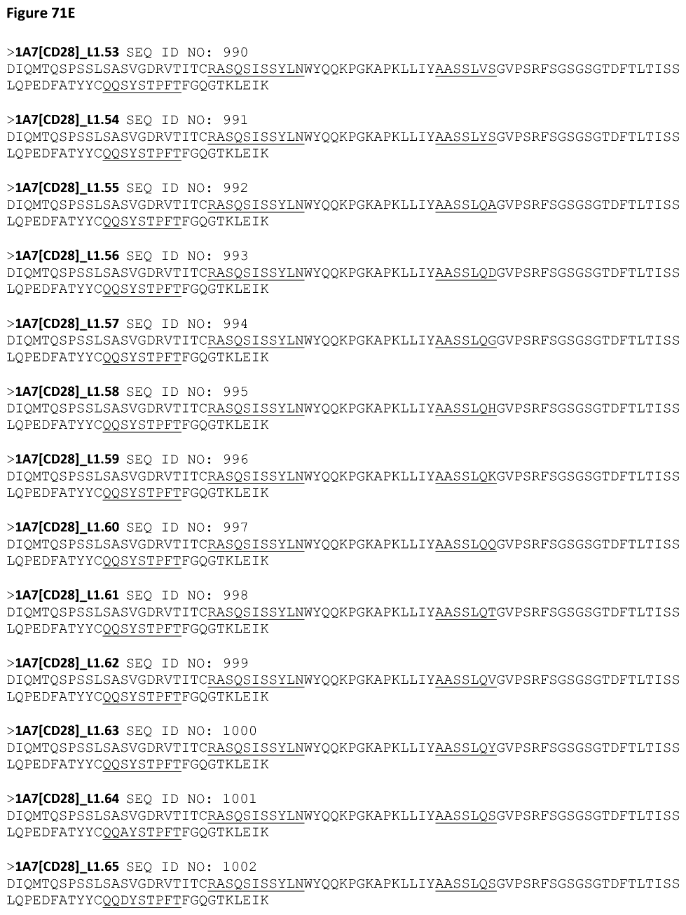

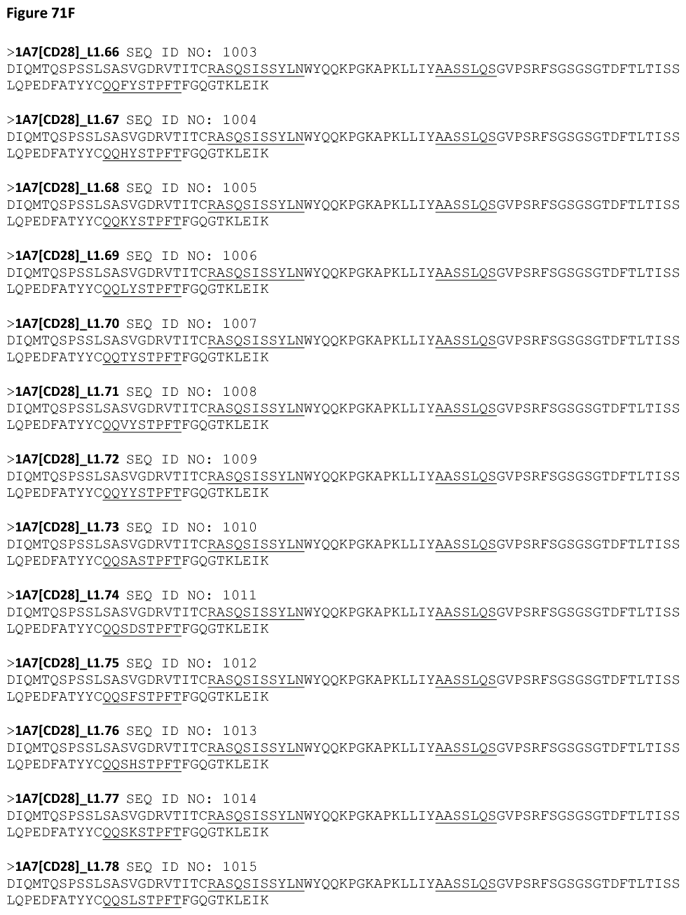

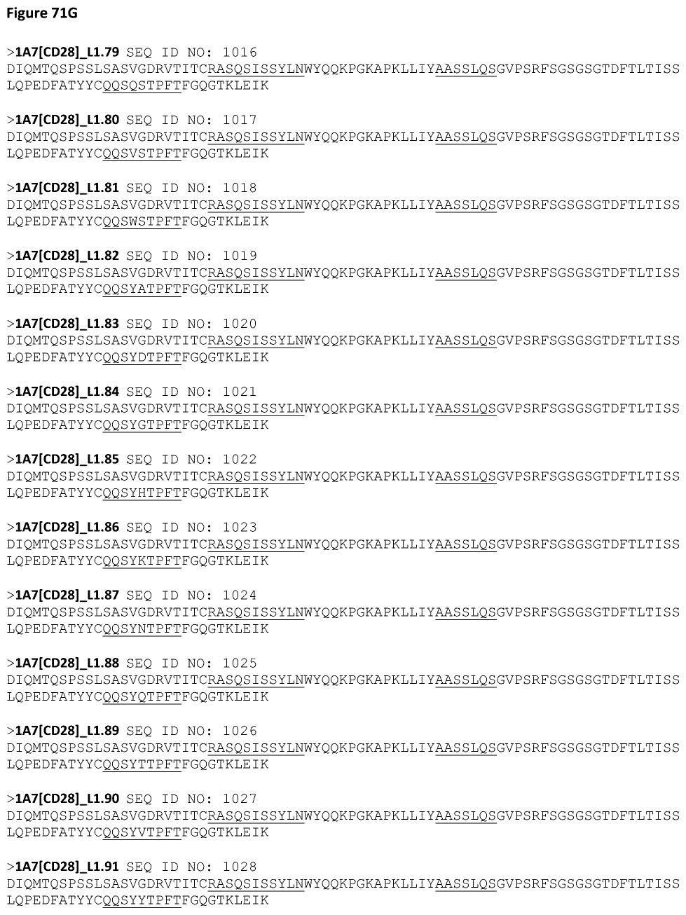

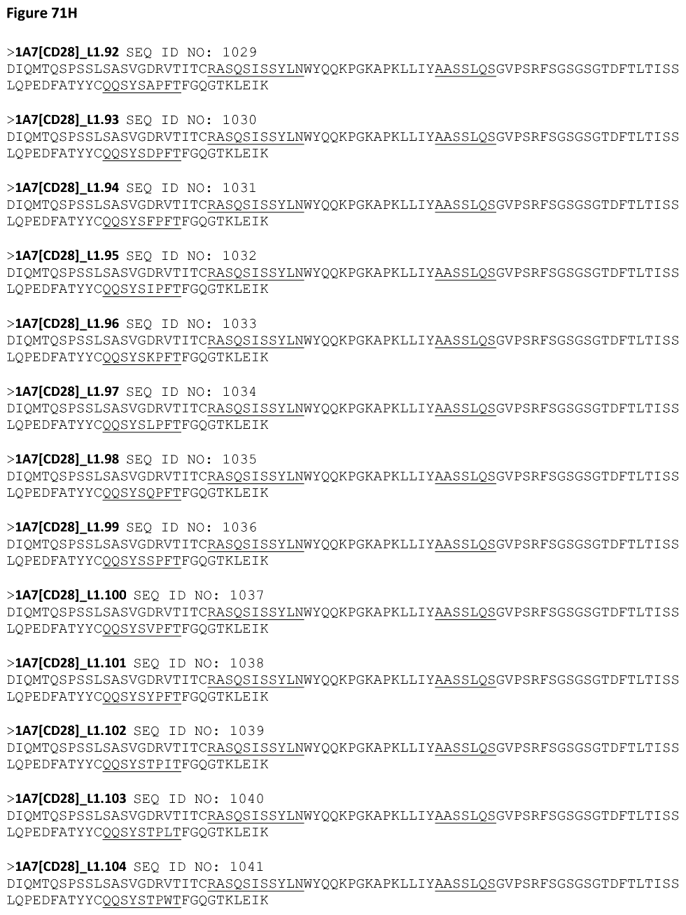

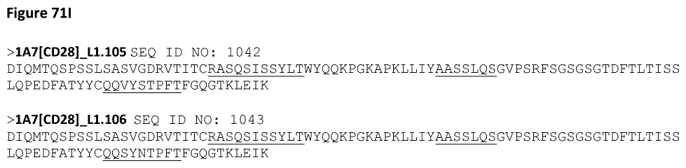

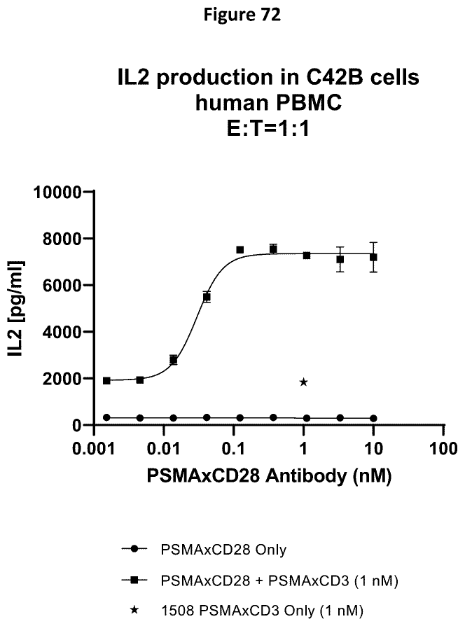

FIG. 11 depicts illustrative sequences of heterodimeric PSMA x CD28 bsAb backbone for use in the 2+1 mAb-scFv format. The format depicted here is based on heterodimeric Fc backbone 1 as depicted in FIGS. 10A-10E, except further including G446_ on monomer 1 (−) and G446_/K447_ on monomer 2 (+). It should be noted that any of the additional backbones depicted in FIGS. 10A-10E may be adapted for use in the 2+1 mAb-scFv format with or without including K447_ on one or both chains. It should be noted that these sequences may further include the M428L/N434S variants or M252Y/S254T/T256E half-life extension variants.

FIG. 12 depicts sequences for “CH1” that find use in embodiments of PSMA x CD28 bsAbs.

FIG. 13 depicts sequences for “hinge” that find use in embodiments of PSMA x CD28 bsAbs.

FIG. 14 depicts the constant domain of the cognate light chains that find use in the subject PSMA x CD28 bsAbs that utilize a Fab binding domain.

FIG. 15 depicts the variable heavy and variable light chain sequences for 1A7, an exemplary phage-derived CD28 binding domain, as well as the sequences for XENP28428, an anti-CD28 mAb based on 1A7 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 16 depicts the sequences for affinity-optimized variable heavy domains from anti-CD28 clone 1A7. It should be noted that the variable heavy domains can be paired with any of the other variable light domains depicted herein, including those depicted in FIGS. 17, 18A-18C and 21A-21H (e.g. 1A7_H1.1_L1.71 as utilized in XENP37559).

FIG. 17 depicts the sequences for affinity-optimized variable light domain from anti-CD28 clone 1A7. It should be noted that the variable light domains can be paired with any of the other variable light domains depicted herein, including those depicted in FIGS. 16, 18 and 21 (e.g. 1A7_H1.1_L1.71 as utilized in XENP37559).

FIGS. 18A-18C depicts the sequence for illustrative affinity-optimized 1A7 VH/VH pairs. It should be noted that these pairs may be formatted as Fabs or as scFvs. Additionally, in the scFv format, these pairs may be formatted in the VHVL orientation or the VLVH orientation.

FIGS. 19A-19B depicts consensus framework regions (FR) and complementarity determining regions (CDRs) (as in Kabat) for anti-CD28 clone 1A7 variable heavy and variable light domain variants.

FIG. 20 depicts illustrative affinity-engineered 1A7 VH/VL pairs and their binding affinities in the context of scFvs (in the context of 1+1 Fab-scFv-Fc bsAb format).

FIGS. 21A-21H depicts the variable heavy and variable light chain sequences for additional CD28 binding domains which find use in the PSMA x CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 22 depicts the sequences for XENP27181, a bivalent anti-CD28 mAb based on HuTN228 binding domain and IgG1 backbone with E233P/L234V/L235A/G236del/S267K ablation variant; and XENP29154 which is an in-house produced version of TGN1412.

FIG. 23 depicts binding of illustrative bivalent anti-CD28 mAbs based on phage-derived clones on human PBMCs. The data show that the phage campaign generated CD28 binding domains having weaker maximum binding than prior art HuTN228 (which is related to the humanized CD28 binding domains described in Example 1A).

FIGS. 24A-24M depicts bispecific formats of the present invention. FIG. 24A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 24B depicts the “2+1 Fab2-scFv-Fc” format, with a first Fab arm and a second Fab-scFv arm, wherein the Fab binds a first antigen and the scFv binds second antigen. The 2+1 Fab2-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 24C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 24D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 24E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen. Additional bispecific formats include F) dual scFv, G) one-arm scFv-mAb, H) scFv-mAb, I) bispecific mAb, J) one-arm central-scFv, K) mAb-Fv, L) central-Fv, and M) trident.

FIGS. 25A-25E depicts the variable heavy and variable light chain sequences for exemplary PSMA binding domains which find use in the PSMA x CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 26A-26B depicts the variable heavy and variable light chain sequences for additional PSMA binding domains which find use in the PSMA x CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 27A-27B depicts A) classic T cell/APC interaction and B) replication of the classic T cell/APC interaction by combining CD3 bispecific antibodies with CD28 bispecific antibodies. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells. In contrast in treatment with CD3 bispecifics, only the first signal is provided. The CD28 signal may be provided by a CD28 bispecific with the idea to promote activation and proliferation through CD28 costimulation. In some embodiments, TAA1 and TAA2 may be different antigens. In some embodiments, TAA1 and TAA2 may be same antigen but different epitopes. In some embodiments, TAA1 and TAA2 may be same antigen and same epitope.

FIGS. 28A-28C depicts sequences for illustrative PSMA x CD3 bsAbs that may be combined with the PSMA x CD28 bsAbs of the invention.

FIGS. 29A-29RR depict the sequences for illustrative PSMA x CD28 bsAbs in the 1+1 Fab-scFv-Fc format using Platform X. Although these sequences utilize Platform X, they can use Platform J or any other suitable backbones including those depicted in FIGS. 10A-10E. CDRs are underlined, and slashes indicate the border(s) between the variable regions, Fc regions, and constant domains. It should be noted that the PSMA x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIGS. 30A-30L depict the sequences for illustrative PSMA x CD28 bsAbs in the 1+1 Fab-scFv-Fc format using Platform J. Although these sequences utilize Platform J, they can use Platform X or any other suitable backbones including those depicted in FIGS. 10A-10E. CDRs are underlined, and slashes indicate the border(s) between the variable regions, Fc regions, and constant domains. It should be noted that the PSMA x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M252Y/S254T/T256E variants in one or preferably both Fc domains, which including M252Y/S254T/T256E results in longer half-life in serum.

FIG. 31 depicts the release of IFNγ from human PBMCs treated with air-dried XENP28428 (anti-CD28 clone 1A7), TGN1412 (XENP29154), or negative control PBS.

FIGS. 32A-32B depicts A) Redirected T Cell Cytotoxicity (RTCC) (as indicated by decrease in target cell count over time) and B) T cell proliferation (as indicated by increase in T cell area over time) after incubating C42B-NLR (PSMA^{hi}) cells with T cells (E:T 1:1) and 1 μg/mL B7H3 x CD3 bsAb alone or in combination with 1 μg/mL prototype PSMA x CD28 bsAbs XENP37902 or XENP37903. The data show that combination with PSMA x CD28 bsAb enhances both RTCC and T cell proliferation in the presence of PSMA^{hi} cells. Notably, the low affinity CD28 binding domain in XENP37902 is sufficient to enhance activity.

FIGS. 33A-33B depicts A) RTCC (as indicated by decrease in target cell count over time) and B) T cell proliferation (as indicated by increase in T cell area over time) after incubating DU145 (PSMA^{null}) cells with T cells (E:T 1:1) and 1 μg/mL B7H3 x CD3 bsAb alone or in combination with prototype 1 μg/mL PSMA x CD28 bsAbs XENP37902 or XENP37903. The data show that combination with PSMA x CD28 bsAb does not enhance RTCC or T cell proliferation in the presence of PSMA^{null} cells.

FIGS. 34A-34B depicts A) RTCC (as indicated by decrease in target cell count over time) and B) T cell proliferation (as indicated by increase in T cell area over time) after incubating C42B-NLR (PSMA^{hi}) cells with T cells (E:T 1:1) and 1 μg/mL PSMA x CD3 bsAb alone or in combination with 1 μg/mL prototype PSMA x CD28 bsAbs XENP37902 or XENP37903. It should be noted that the PSMA x CD3 and PSMA x CD28 bsAbs bind non-competing PSMA epitopes. The data show that combination with PSMA x CD28 bsAb enhances both RTCC and T cell proliferation in the presence of PSMA^{hi} cells. Notably, the low affinity CD28 binding domain in XENP37902 is sufficient to enhance activity.

FIGS. 35A-35B depicts A) RTCC (as indicated by decrease in target cell count over time) and B) T cell proliferation (as indicated by increase in T cell area over time) after incubating DU145-NLR (PSMA^{null}) cells with T cells (E:T 1:1) and 1 μg/mL PSMA x CD3 bsAb alone or in combination with 1 μg/mL prototype PSMA x CD28 bsAbs XENP37902 or XENP37903. It should be noted that the PSMA x CD3 and PSMA x CD28 bsAbs bind non-competing PSMA epitopes. The data show that combination with PSMA x CD28 bsAb does not enhance RTCC or T cell proliferation in the presence of PSMA^{null} cells.

FIGS. 36A-36B depicts induction of IL-2 secretion by A) XENP37902 PSMA x CD28 having 230 nM CD28 binding affinity and B) XENP37903 PSMA x CD28 having 37 nM CD28 binding affinity in combination with an illustrative non-competing PSMA x CD3 bsAb incubated with T cells and PC3 cells transfected with varying surface PSMA densities. The data show that the PSMA x CD28 bsAb enhanced cytokine secretion in the presence of PSMA^{hi} and PSMA^{med} cell lines. Notably, the low affinity CD28 binding domain in XENP37902 is sufficient to enhance activity.

FIGS. 37A-37B depicts induction of IL-2 secretion by A) XENP37902 PSMA x CD28 having 230 nM CD28 binding affinity and B) XENP37903 PSMA x CD28 having 37 nM CD28 binding affinity in combination with an illustrative competing PSMA x CD3 bsAb (i.e. binding the same PSMA epitope) incubated with T cells and PC3 cells transfected with varying surface PSMA densities. The data show that the PSMA x CD28 bsAb enhanced cytokine secretion in the presence of PSMA^{hi} and PSMA^{med} cell lines. Notably, a hooking effect is observed at higher concentrations.

FIG. 38 depicts binding to A) C42B-NLR (PSMA^{hi}) and B) 22Rv1-NLR (PSMA^{med}) cells by PSMA x CD28 bsAbs incorporating a 1A7_H1_L1.71 180 nM CD28 binding domain and various PSMA binding domains.

FIG. 39 depicts induction of IL-2 secretion by PSMA x CD28 bsAbs incorporating a 1A7_H1_L1.71 180 nM CD28 binding domain and various PSMA binding domains in combination with an illustrative B7H3 x CD3 bsAb incubated with T cells and 22Rv1-NLR (PSMA^{med}) cells. The data show a range of potency consistent with cell binding.

FIG. 40 depicts induction of IL-2 secretion by PSMA x CD28 bsAbs incorporating a 1A7_H1_L1.71 180 nM CD28 binding domain and various PSMA binding domains in combination with 1391 PSMA x CD3 incubated with T cells and 22Rv1-NLR (PSMA^{med}) cells.

FIG. 41 depicts induction of IL-2 secretion by PSMA x CD28 bsAbs incorporating A10v2 PSMA binding domain and CD28 binding domains of varying affinities in combination with 1391 PSMA x CD3 (constant 1 μg/mL PSMA x CD28 and dose titration of PSMA x CD3) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 42 depicts induction of IL-2 secretion by PSMA x CD28 bsAbs incorporating DO1v2 PSMA binding domain and CD28 binding domains of varying affinities in combination with 1391 PSMA x CD3 (constant 1 μg/mL PSMA x CD28 and dose titration of PSMA x CD3) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 43 depicts induction of IL-2 secretion by PSMA x CD28 bsAbs incorporating A10v2 PSMA binding domain and CD28 binding domains of varying affinities in combination with 1391 PSMA x CD3 (constant 1 μg/mL PSMA x CD3 and dose titration of PSMA x CD28) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 44 depicts induction of IL-2 secretion by PSMA x CD28 bsAbs incorporating DO1v2 PSMA binding domain and CD28 binding domains of varying affinities in combination with 1391 PSMA x CD3 (constant 1 μg/mL PSMA x CD3 and dose titration of PSMA x CD28) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 45 depicts induction of IL-2 secretion by PSMA x CD28 bsAbs incorporating various PSMA binding domain and CD28 binding domains of varying affinities in combination with 1391 PSMA x CD3 (constant 1 μg/mL PSMA x CD3 and dose titration of PSMA x CD28) incubated with T cells and 22Rv1-NLR (PSMA^{med}) at A) 1:1 and B) 0.1:1 effector:target ratios.

FIG. 46 depicts induction of IL-2 secretion by PSMA x CD28 bsAbs incorporating various PSMA binding domain and CD28 binding domains of varying affinities in combination with 1391 PSMA x CD3 (constant 1 μg/mL PSMA x CD3 and dose titration of PSMA x CD28) incubated with T cells and PSMA-PC3 (˜50K) at A) 1:1 and B) 0.1:1 effector:target ratios.

FIG. 47 depicts induction of T cell proliferation by PSMA x CD28 bsAbs incorporating various PSMA binding domain and CD28 binding domains of varying affinities in combination with 1391 PSMA x CD3 (constant 1 μg/mL PSMA x CD3 and dose titration of PSMA x CD28) incubated with T cells and 22Rv1-NLR (PSMA^{med}) at 1:1 effector:target ratios.

FIG. 48 depicts induction of RTCC (as indicated by decrease in target cell count over time) after incubating 22Rv1-NLR (PSMA^{med}) cells with T cells (E:T 1:1) and A) 0.3 mg/mL, B) 1 ng/mL, C) 10 ng/ml, or D) 2.5 μg/mL 1391 PSMA x CD3 alone or in combination with 1 μg/mL PSMA x CD28 bsAbs incorporating various PSMA binding domain and CD28 binding domains of varying affinities. The data show that combination with PSMA x CD28 bsAb enhances RTCC. Notably at higher 2.5 μg/mL concentration, high affinity CD28 binding domain as in XENP39234 and XENP39233 does not perform as well as low affinity CD28 binding domains as in XENP38936 and XENP39232.

FIG. 49 depicts induction of IL-2 secretion by PSMA x CD28 bsAbs incorporating A10v2 PSMA binding domain and CD28 binding domains of additional lower affinities in combination with 1391 PSMA x CD3 (constant 1 μg/mL PSMA x CD3 and dose titration of PSMA x CD28) incubated with T cells and A) PSMA-PC3 (˜100K) or B) 22Rv1-NLR (PSMA^{med}).

FIG. 50 depicts induction of IL-2 secretion by PSMA x CD28 bsAbs incorporating A10v2 PSMA binding domain and CD28 binding domains of varying affinities in combination with 1391 PSMA x CD3 (constant 1 μg/mL PSMA x CD3 and dose titration of PSMA x CD28) incubated with T cells and PSMA-PC3-NLR (˜50K PSMA), PSMA-PC3-NLR (˜3K PSMA), or DU145-NLR (OK PSMA).

FIG. 51 depicts induction of IL-2 secretion by PSMA x CD28 bsAbs incorporating A10v2 PSMA binding domain and CD28 binding domains of varying affinities built on Platform X or Platform J in combination with 1391 PSMA x CD3 (constant 1 μg/mL PSMA x CD3 and dose titration of PSMA x CD28) incubated with T cells and PSMA-PC3 (˜50K).

FIG. 52 depicts induction of IL-2 secretion, IFNγ secretion, and RTCC by XENP39234 and XENP38936 in combination with A) a B7H3 x CD3 bsAb and B) XENP33063 PSMA x CD3 bsAb. PSMA x CD28 combine more potently with tumor antigen matched CD3 bispecific.

FIG. 53 depicts the change in tumor volume (as determined by caliper measurements) over time in PSMA-transfected PC3 (˜100K) and huPBMC-engrafted NSG-DKO mice dosed with XENP33063 PSMA x CD3 or B7H3 x CD3 bsAbs alone or in combination with XENP38936 (PSMA x CD28 180 nM) or XENP39231 (PSMA x CD28 1 μM).

FIG. 54 depicts tumor volume (as determined by caliper measurements) on A) Day 16 and B) Day 19 in PSMA-transfected PC3 (˜100K) and huPBMC-engrafted NSG-DKO mice dosed with PSMA x CD3 or B7H3 x CD3 bsAbs alone or in combination with XENP38936 (PSMA x CD28 180 nM) or XENP39231 (PSMA x CD28 1 μM).

FIG. 55 depicts A) human CD4+ and B) human CD8+ cell counts on Day 14 in PSMA-transfected PC3 (˜100K) and huPBMC-engrafted NSG-DKO mice dosed with PSMA x CD3 or B7H3 x CD3 bsAbs alone or in combination with XENP38936 (PSMA x CD28 180 nM) or XENP39231 (PSMA x CD28 1 μM).

FIG. 56 depicts induction of IL-2 secretion by PSMA x CD28 bsAbs incorporating a 1A7_H1_L1.71 180 nM CD28 binding domain and various PSMA binding domains in combination with 1508 PSMA x CD3 incubated with T cells and 22Rv1-NLR (PSMA^{med}) cells.

FIG. 57 depicts induction of IL-2 secretion by PSMA x CD28 bsAbs incorporating A10v2 PSMA binding domain and CD28 binding domains of varying affinities in combination with 1508 PSMA x CD3 (constant 1 μg/mL PSMA x CD28 and dose titration of PSMA x CD3) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 58 depicts induction of IL-2 secretion by PSMA x CD28 bsAbs incorporating DO1v2 PSMA binding domain and CD28 binding domains of varying affinities in combination with 1508 PSMA x CD3 (constant 1 μg/mL PSMA x CD28 and dose titration of PSMA x CD3) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 59 depicts induction of IL-2 secretion by PSMA x CD28 bsAbs incorporating A10v2 PSMA binding domain and CD28 binding domains of varying affinities in combination with 1508 PSMA x CD3 (constant 1 μg/mL PSMA x CD3 and dose titration of PSMA x CD28) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 60 depicts induction of IL-2 secretion by PSMA x CD28 bsAbs incorporating DO1v2 PSMA binding domain and CD28 binding domains of varying affinities in combination with 1508 PSMA x CD3 (constant 1 μg/mL PSMA x CD3 and dose titration of PSMA x CD28) incubated with T cells and A) C42B-NLR (PSMA^{hi}), B) PC3-PSMA (˜100K), C) 22Rv1-NLR (PSMA^{med}), and D) PC3-PSMA (˜50K) cells.

FIG. 61 depicts induction of IL-2 secretion by PSMA x CD28 bsAbs incorporating various PSMA binding domain and CD28 binding domains of varying affinities in combination with 1508 PSMA x CD3 (constant 1 μg/mL PSMA x CD3 and dose titration of PSMA x CD28) incubated with T cells and 22Rv1-NLR (PSMA^{med}) at A) 1:1 and B) 0.1:1 effector:target ratios.

FIG. 62 depicts induction of IL-2 secretion by PSMA x CD28 bsAbs incorporating various PSMA binding domain and CD28 binding domains of varying affinities in combination with 1508 PSMA x CD3 (constant 1 μg/mL PSMA x CD3 and dose titration of PSMA x CD28) incubated with T cells and PSMA-PC3 (˜50K) at A) 1:1 and B) 0.1:1 effector:target ratios.

FIG. 63 depicts induction of IL-2 secretion by PSMA x CD28 bsAbs incorporating A10v2 PSMA binding domain and CD28 binding domains of additional lower affinities in combination with 1508 PSMA x CD3 (constant 1 μg/mL PSMA x CD3 and dose titration of PSMA x CD28) incubated with T cells and A) PSMA-PC3 (˜100K) or B) 22Rv1-NLR (PSMA^{med}).

FIG. 64 depicts induction of IL-2 secretion by PSMA x CD28 bsAbs incorporating A10v2 PSMA binding domain and CD28 binding domains of varying affinities in combination with 1508 PSMA x CD3 (constant 1 μg/mL PSMA x CD3 and dose titration of PSMA x CD28) incubated with T cells and PSMA-PC3-NLR (˜50K PSMA), PSMA-PC3-NLR (˜3K PSMA), or DU145-NLR (OK PSMA).

FIG. 65 depicts induction of IL-2 secretion by PSMA x CD28 bsAbs incorporating A10v2 PSMA binding domain and CD28 binding domains of varying affinities built on Platform X or Platform J in combination with 1508 PSMA x CD3 (constant 1 μg/mL PSMA x CD3 and dose titration of PSMA x CD28) incubated with T cells and PSMA-PC3 (˜50K).

FIG. 66 depicts binding to C42B (PSMA^{hi}) by PSMA x CD28 bsAbs incorporating A10v2 PSMA binding domain and CD28 binding domains of varying affinities built on Platform X or Platform J.

FIG. 67 depicts the sequences for illustrative variable heavy domains from anti-CD28 clone 1A7 with an introduced cysteine to allow for “stapling” of the scFv. It should be noted that the variable heavy domains can be paired with any of the other CD28 binding domain variable light domains depicted herein, including those depicted in FIG. 68 (e.g. as utilized in C28PB405, C28PB404, C28PB403, C28PB402, C28PB401, C28PB400, and C28PB397).

FIG. 68 depicts the sequences for illustrative variable light domain from anti-CD28 clone 1A7 with an introduced cysteine to allow for “stapling” of the scFv. It should be noted that the variable heavy domains can be paired with any of the other CD28 binding domain variable heavy domains depicted herein, including those depicted in FIG. 67 (e.g. as utilized in C28PB405, C28PB404, C28PB403, C28PB402, C28PB401, C28PB400, and C28PB397).

FIGS. 69A-69C depicts the sequence for illustrative stapled 1A7 VH/VL pairs. These pairs may be formatted in the VHVL orientation or the VLVH orientation.

FIGS. 70A-70F depicts additional sequences for affinity-optimized variable heavy domains from anti-CD28 clone 1A7. It should be noted that the variable heavy domains can be paired with any of the other CD28 binding domain variable light domains depicted herein, including those depicted in FIGS. 71A-71I.

FIGS. 71A-71I depicts additional sequences for affinity-optimized variable light domains from anti-CD28 clone 1A7. It should be noted that the variable light domains can be paired with any of the other CD28 binding domain variable heavy domains depicted herein, including those depicted in FIGS. 70-70F.

FIG. 72 shows addition of PSMAxCD28 increased IL2 production of PSMAxCD3 in a dose dependent manner in C42B cells using human PBMCs.

### DETAILED DESCRIPTION

### I. Overview

Prostate cancer (PC) is one of the most prevalent cancers in men, and end stage (castration-resistant prostate cancer) has no curative treatment option. Prostate Specific Membrane Antigen (PSMA), a type II transmembrane protein with a large extracellular domain, has long generated interest as a therapeutic target. PSMA is highly overexpressed in PC compared to normal tissue, and its expression has been shown to correlate with malignancy. Previous attempts to target PSMA include antibody-based radiotherapy and antibody drug conjugates, which have shown some success but can be hampered by the inherent toxicity of the modality.

The activation of T cells in the treatment of cancer is being widely investigated. T cells require multiple signals for complete activation and differentiation. As shown in FIG. 27A, Signal 1, promoted by recognition of a peptide-MHC (pMHC) complex by the T cell receptor (TCR), is absolutely required for T cell activation. Signal 2, which synergizes with, and amplifies signal 1, is typically provided by the interaction of the CD28 ligands CD80 and CD86 with CD28 itself. Although CD28 engagement alone is typically inert, when combined with signal 1 activation, it promotes additional activation, survival, and proliferative signals, including IL-2 secretion. As CD80 and CD86 are only naturally expressed by professional antigen-presenting cells (APC), the extent of CD28 costimulation in the tumor setting can be highly variable. Accordingly, the present invention is directed to a novel class of tumor-targeted anti-CD28 x anti-PSMA bispecific antibodies that mimick the CD80/CD86 engagement of CD28, thereby providing an artificial source of signal 2. Notably, signal 1 can either be provided by the natural TCR:pMHC recognition of tumor cells, or it can be provided by combination of the CD28 bispecific with a CD3 bispecific (e.g., anti-CD3 x anti-PSMA), which can mimic signal 1.

Accordingly, provided herein are novel anti-CD28 x anti-PSMA (also referred to as “αCD28 x αPSMA” and sometimes “CD28 x PSMA”) bispecific antibodies and methods of using such antibodies for the treatment of PSMA-associated cancers. In many cases, these bispecific antibodies are heterodimeric. Subject αCD28 x αPSMA antibodies are capable of agonistically binding to CD28 costimulatory molecules on T cells and targeting to PSMA on PSMA-expressing tumor cells. Thus, such antibodies selectively enhance anti-tumor activity at PSMA-expressing tumor sites while minimizing peripheral toxicity. The subject antibodies provided herein are particularly useful for enhancing anti-tumor activity either alone, as a monotherapy, or when used in combination with other anti-cancer therapies as more fully described herein.

Accordingly, in one aspect, provided herein are heterodimeric antibodies that bind to two different antigens, e.g., the antibodies are “bispecific,” in that they bind two different target antigens, generally CD28 and PSMA as described below. These heterodimeric antibodies can bind each of the target antigens either monovalently (e.g., there is a single antigen binding domain such as a variable heavy and variable light domain pair) or bivalently (there are two antigen binding domains that each independently bind the antigen). In some embodiments, the heterodimeric antibody provided herein includes one CD28 binding domain and one PSMA binding domain (e.g., heterodimeric antibodies in the “1+1 Fab-scFv-Fc” format described herein, which are thus bispecific and bivalent). In other embodiments, the heterodimeric antibody provided herein includes one CD28 binding domain and two PSMA binding domains (e.g., heterodimeric antibodies in the “2+1 Fab2-scFv-Fc” formats described herein, which are thus bispecific but trivalent, as they contain three antigen binding domains (ABDs)). The heterodimeric antibodies provided herein are based on the use of different monomers that contain amino acid substitutions (i.e., skew variants”) that “skew” formation of heterodimers over homodimers, as is more fully outlined below. In some embodiments, the heterodimer antibodies are also coupled with purification variants (e.g., “pI variants”) that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

The naming nomenclature of particular antigen binding domains (e.g., PSMA and CD28 binding domains) use a “Hx.xx_Ly.yy” type of format, with the numbers being unique identifiers to particular variable chain sequences. Thus, for example, the CD28 binding domain “1A7[CD28]_H1_L1” (FIG. 15) includes a variable heavy domain, H1, and a variable light domain L1. In the case that these sequences are used as scFvs, the designation “H1_L1”, indicates that the binding domain includes a variable heavy domain “H1” combined with a variable light domain “L1,” and is in VH-linker-VL orientation, from N- to C-terminus. This molecule with the identical sequences of the heavy and light variable domains but in the reverse order (VL-linker-VH orientation, from N- to C-terminus) would be designated “L1_H1”. Similarly, different constructs may “mix and match” the heavy and light chains as will be evident from the sequence listing and the Figures.

### III. Definitions

In order that the application may be more completely understood, several definitions are set forth below. Such definitions are meant to encompass grammatical equivalents.

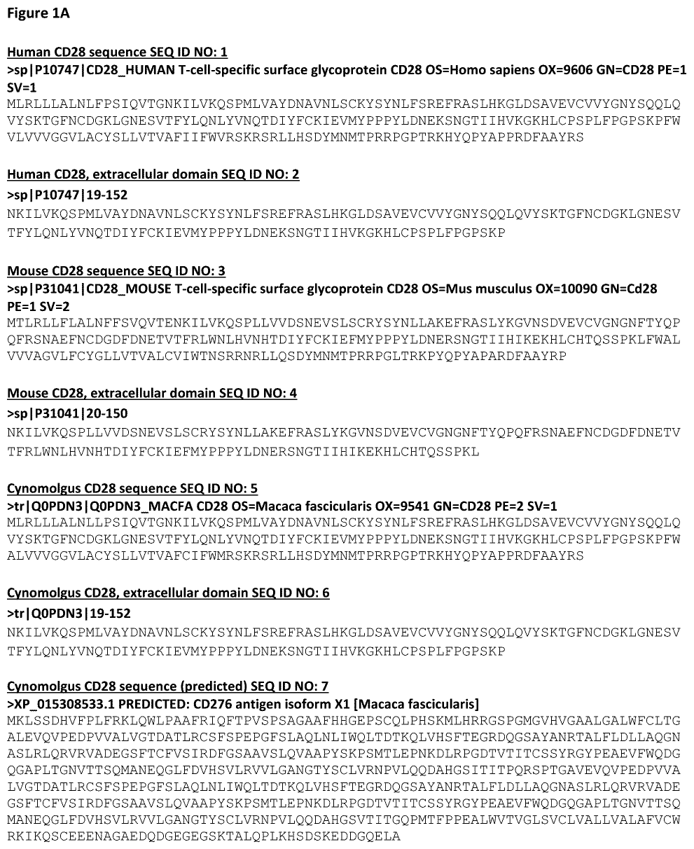

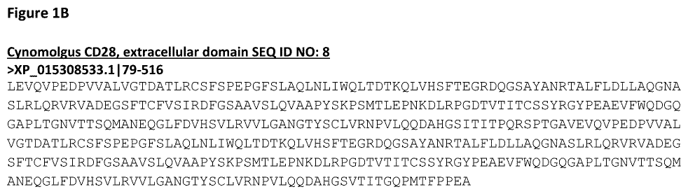

By “CD28,” “Cluster of Differentiation 28,” and “Tp44” (e.g., Genebank Accession Numbers NP_001230006 (human), NP_001230007 (human), NP_006130 (human), and NP_031668 (mouse)) herein is meant a B7 receptor expressed on T cells that provides co-stimulatory signals required for T cell activation and survival. T cell stimulation through CD28 in addition to the T cell receptor (TCR) provides a potent signal for the production of various interleukins. CD28 is the receptor for CD80 (B7.1) and CD86 (B7.2) proteins. CD28 includes an intercellular domain with a YMNM motif (SEQ ID NO: 1044) critical for the recruitment of SH2-domain containing proteins, particularly PI3K. CD28 also includes two proline-rich motifs that are able to bind SH3-containing proteins. Exemplary CD28 sequences are depicted in FIGS. 1A-1B. Unless otherwise noted, references to CD28 are to the human CD28 sequence.

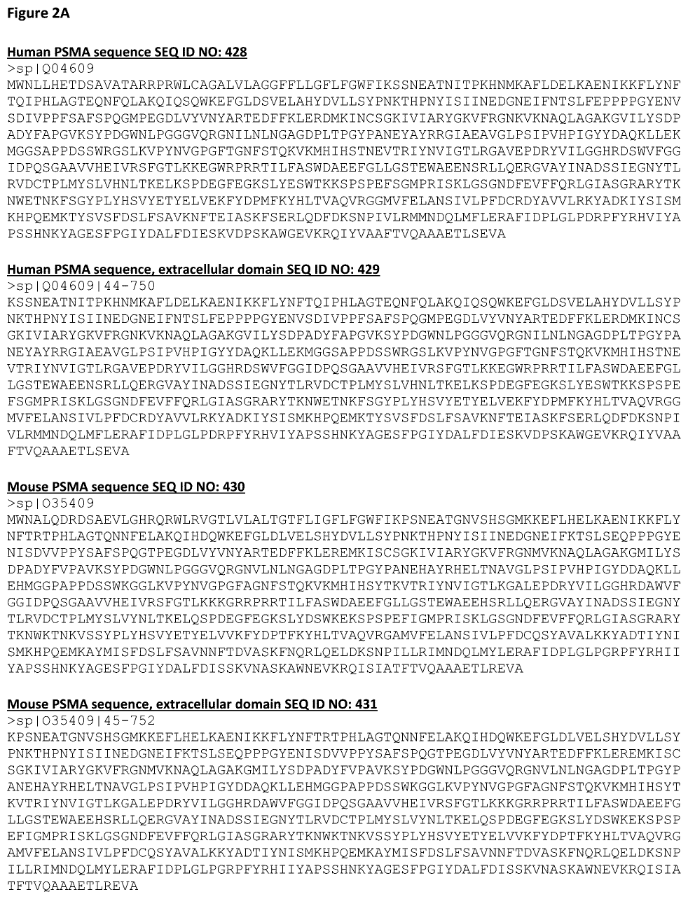

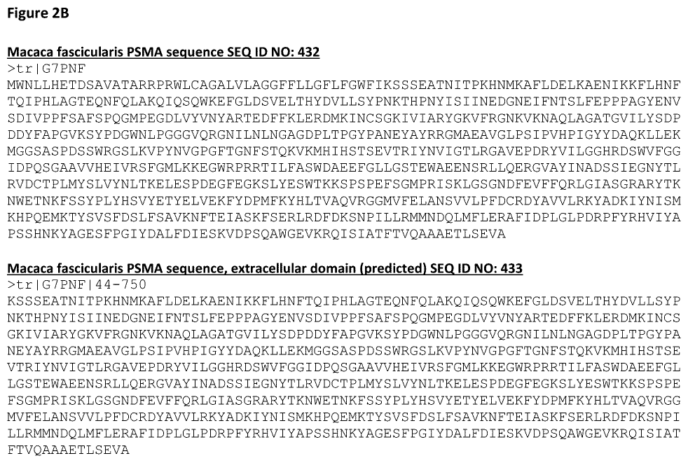

By “PSMA” or “Prostate Specific Membrane Antigen” (e.g., Genebank Accession Number NP 005012.2) herein is meant a type II transmembrane protein that is expressed in prostatic tissues, including primary prostate adenocarcinomas, metastatic prostate cancer, and in the tumor neovasculature of many solid tumors. In prostate cancer (PCa), PSMA is highly expressed in poorly differentiated, highly metastatic prostatic cells and in castrate-resistant models. Exemplary PSMA sequences are disclosed in FIGS. 2A-2B. Unless otherwise noted, references to PSMA are to the human PSMA sequence.

By “B7H3,” “B7-H3,” “B7RP-2,” “CD276,” “Cluster of Differentiation 276,” (e.g., Genebank Accession Numbers NP_001019907 (human), NP_001316557 (human), NP_001316558 (human), NP_079516 (human), and NP_598744 (mouse)) herein is meant a type-1 transmembrane protein that is a member of the B7 family possessing an ectodomain composed of a single IgV-IgC domain pair. B7H3 is an immune checkpoint molecule and is aberrantly overexpressed in many types of cancers. Unless otherwise noted, references to B7H3 are to the human B7H3 sequence.

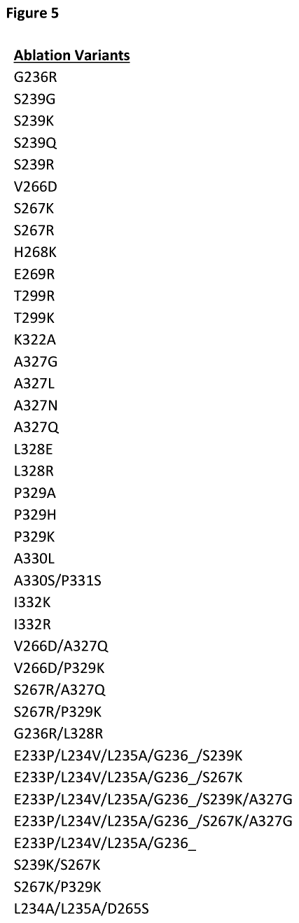

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 5, which generally are added to both monomers.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

Other useful antibody formats include, but are not limited to, the “1+1 Fab-scFv-Fc,” and “2+1 Fab2-scFv-Fc” formats provided herein (sec, e.g., FIGS. 24A-24M). Additional useful antibody formats include, but are not limited to, “1+1 common light chain,” and “2+1 common light chain,” “mAb-Fv,” “mAb-scFv,” “central-Fv”, “one armed scFv-mAb,” “scFv-mAb,” “dual scFv,” and “trident” format antibodies (FIGS. 24A-24M). See also, US20180127501A1, which is incorporated by reference herein, particularly in pertinent part relating to antibody formats (see, e.g., FIG. 2 of US20180127501A1).

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., PSMA or CD28) as discussed herein. As is known in the art, these CDRs are generally present as a first set of variable heavy CDRs (vhCDRs or VHCDRs) and a second set of variable light CDRs (vICDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N-to C-terminus (vh-linker-vl or vl-linker-vh). In the sequences depicted in the sequence listing and in the figures, the order of the vh and vl domain is indicated in the name, e.g., H.X_L. Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233— or E233 #, E233( )or E233del designates a deletion of glutamic acid at position 233. Additionally, EDA233— or EDA233 #designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol.48:443 [homology alignment algorithm], Pearson, W.R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000— or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000— or more times greater for the epitope relative to a control, where KA or Ka refers to an association rate of a particular antibody-antigen interaction. Binding affinity is generally measured using a Biacore, SPR or BLI assay.

### IV. Anti-CD28 x Anti-PSMA Antibodies

In one aspect, provided herein are novel anti-CD28 x anti-PSMA antibodies In some embodiments, the anti-CD28 x anti-PSMA antibodies described herein are capable of agonistically binding to CD28 costimulatory molecules on T cells and PSMA on tumor cells. Such antibodies selectively enhance anti-tumor activity at PSMA-associated tumor sites while minimizing peripheral toxicity. The subject antibodies provided herein are particularly useful in combination with other anti-cancer therapies, including, for example, multivalent antibodies for the treatment of prostate cancers.

The anti-CD28 x anti-PSMA antibodies are multivalent and include at least two antigen binding domains (ABDs), wherein at least one antigen binding domain is a CD28 binding domain and at least one antigen binding domain is a PSMA binding domain. Any suitable CD28 binding domain and PSMA binding domain can be included in the subject anti-CD28 x anti-PSMA antibodies, including, for example, the CD28 binding domains and PSMA binding domains provided herein.

The antigen binding domains provided herein generally include a variable heavy domain (VH) having VH-CDR1, VH-CDR-2, and VH-CDR-3; and a variable light domain (VL), and a variable light domain (VL) having VL-CDR1, VL-CDR-2, and VL-CDR-3.

In addition, as discussed above, the numbering used in the sequence listing and figures for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 90, 95 or 99% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85 90, 95 or 99% identity to a human germline sequence selected from those listed in FIG. 1 of U.S. Pat. No. 7,657,380.

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain).

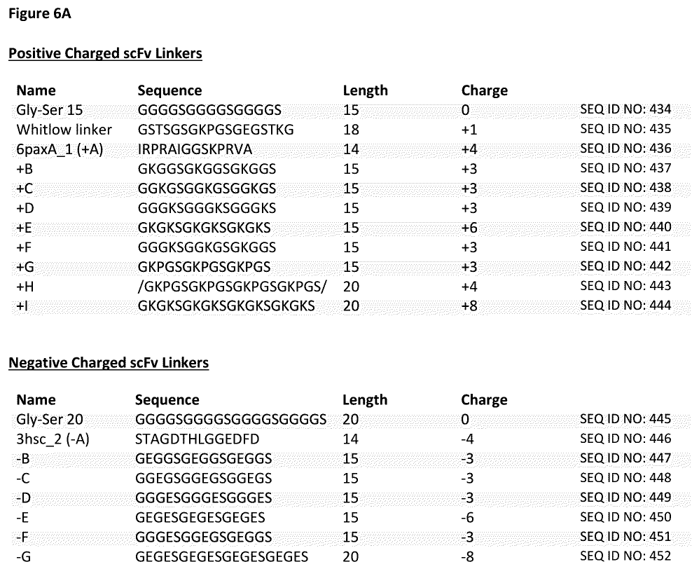

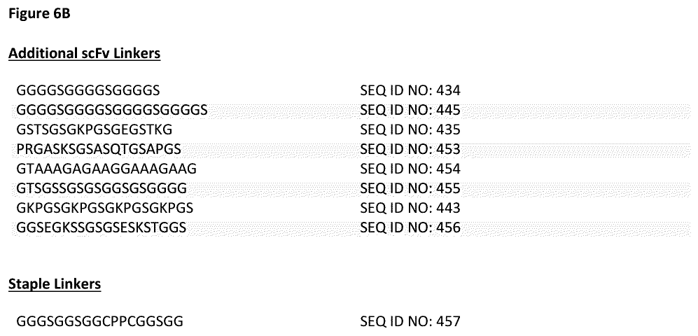

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in FIGS. 6A-6B.

Useful CD28 binding domains and PSMA binding domains that can be included in the subject anti-CD28 x anti-PSMA antibodies are further detailed herein.

In some embodiments, the anti-CD28 x anti-PSMA antibody is a bispecific antibody. In some embodiments, the anti-CD28 x anti-PSMA antibody is a bivalent antibody. In some embodiments, the anti-CD28 x anti-PSMA antibody is a trivalent antibody. In some embodiments, the anti-CD28 x anti-PSMA antibody is a bispecific, bivalent antibody. In some embodiments, the anti-CD28 x anti-PSMA antibodies include one CD28 binding domain and one PSMA binding domain. In exemplary embodiments, the anti-CD28 x anti-PSMA antibody is a bispecific, trivalent antibody. In some embodiments, the anti-CD28 x anti-PSMA antibodies include one CD28 binding domain and two PSMA binding domains.

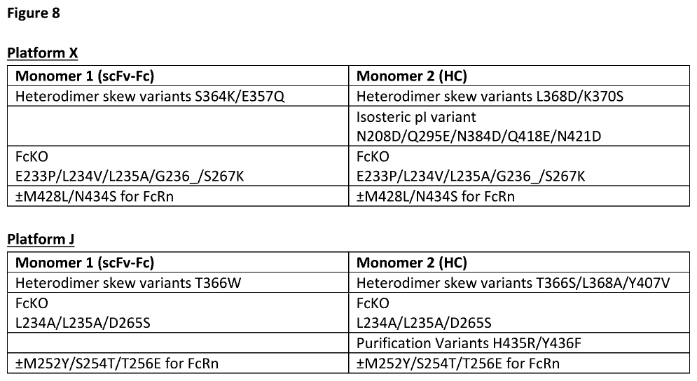

The anti-CD28 x anti-PSMA antibodies provided herein can be in any useful format, including, including, for example, canonical immunoglobulin, as well as the “1+1 Fab-scFv-Fc,” “2+1 Fab2-scFv-Fc,” described herein (FIGS. 24A-24M). Additional useful formats include, but are not limited to: “1+1 common light chain,” and “2+1 common light chain,” “mAb-Fv,” “mAb-scFv,” “central-Fv”, “one armed scFv-mAb,” “scFv-mAb,” “dual scFv,” and “trident” formats provided herein (see, e.g., FIGS. 24A-24M). See also, US20180127501A1, which is incorporated by reference herein, particularly in pertinent part relating to antibody formats (sec, e.g., FIG. 2). In some embodiments, the anti-CD28 x anti-PSMA antibodies are heterodimeric bispecific antibodies that include variant Fc domains having any of the heterodimerization skew variants, pI variants and/or ablation variants described herein. Sec, e.g. FIG. 8.

Note that unless specified herein, the order of the antigen list in the name does not confer structure; that is an anti-PSMA x anti-CD28 1+1 Fab-scFv-Fc antibody can have the scFv bind to PSMA or CD28, although in some cases, the order specifies structure as indicated.

The anti-CD28 x anti-PSMA antibodies provided herein further include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

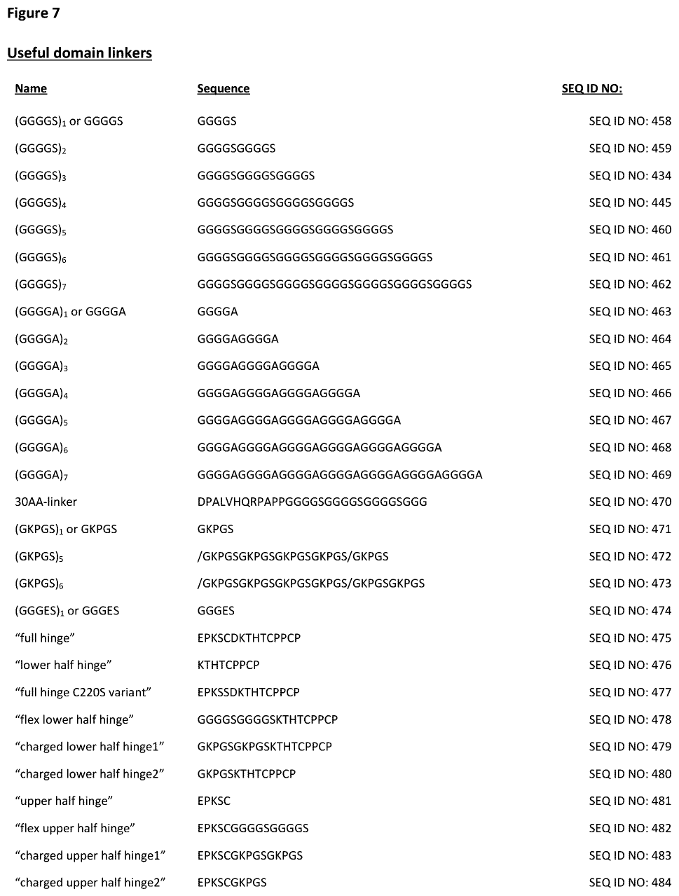

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 1045), (GGGGS)n (SEQ ID NO: 1046), and (GGGS)n (SEQ ID NO: 1047), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker”, used to link any two domains as outlined herein together. For example, in the 2+1 Fab2-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 1045), (GGGGS)n (SEQ ID NO: 1046), and (GGGS)n (SEQ ID NO: 1047), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 7.

In some embodiments, the linker is a scFv linker that is used to covalently attach the VH and VL domains as discussed herein. In many cases, the scFv linker is a charged scFv linker, a number of which are shown in FIGS. 6A-6B. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen. Charged domain linkers can also be used to increase the pI separation of the monomers of the invention as well, and thus those included in FIGS. 6A-6B can be used in any embodiment herein where a linker is utilized. In some embodiments, the scFv is a “stapled” scFv that includes a “staple linker.” “Stapled” scFvs that exhibit improved stability and/or reduced aggregation are further described in detail herein. Exemplary staple linkers that are useful for inclusion in such “stapled” scFvs are provided in FIGS. 6A-6B.

In some embodiments, wherein an scFv is included in the anti-CD28 x anti-PSMA antibody (e.g., the 1+1 Fab-scFv-Fc format or 2+1 Fab2-scFv-Fc format antibody), the scFv includes “staple” modifications that improve scFv stability and/or reduces aggregation. In exemplary embodiments, such “stapled” scFvs include: a) a first disulfide bond between a structurally conserved surface exposed VH cysteine and a first scFv linker cysteine; b) a second disulfide bond between a structurally conserved surface exposed VL cysteine and a second scFv linker cysteine; or c) the first disulfide bond between the structurally conserved surface exposed VH cysteine and the first scFv linker cysteine and the second disulfide bond between the structurally considered surface exposed VL cysteine and the second scFv linker cysteine. Exemplary scFv “staple linkers” for inclusion in “stapled” scFvs are provided in FIGS. 6A-6B. Methods for making “stapled” scFvs are described in WO 2021/030657, which is incorporated by reference in its entirety, including pertinent parts relating to methods for making “stapled” scFvs, and “stapled” scFv compositions.

Exemplary subject anti-CD28 x anti-PSMA antibodies are depicted, for example, in FIGS. 29A-29RR and 30A-30L. During the cell culture production of the anti-CD28 x anti-PSMA antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-CD28 x anti-PSMA antibody is a variant of one of the anti-CD28 x anti-PSMA antibodies depicted in FIGS. 29A-29RR and 30A-30L that includes a deletion of a C-terminal lysine (−K) residues or lysine and glycine (−GK) residues in “chain 1” and/or “chain 2.” In some embodiments, the deletion is G446del and/or K447del. In some embodiments, the anti-CD28 x anti-PSMA antibody is engineered to include a G446del and/or K447del modification in one or both Fc domains of a anti-CD28 x anti-PSMA antibody described herein. In some embodiments, the anti-CD28 x anti-PSMA antibody includes a nautrally occuring G446del and/or K447del modification in one or both Fc domains as compared to an anti-CD28 x anti-PSMA antibody provided herein.

Aspects of the anti-CD28 x anti-PSMA antibodies are further described in detail below.

### A. CD28 Binding Domains

The anti-CD28 x anti-PSMA antibodies provided herein include at least one CD28 binding domain. Any suitable CD28 binding domain can be included in the anti-CD28 x anti-PSMA antibodies provided herein. In exemplary embodiments, the CD28 binding domain is an agonistic CD28 ABDs that advantageously provide T cell costimulatory activity.

As will be appreciated by those in the art, suitable CD28 binding domains can comprise a set of 6 CDRs as depicted in the figures, either as they are underlined or, in the case where a different numbering scheme is used, as described herein and as shown in Table 2, as the CDRs that are identified using other alignments within the variable heavy (VH) domain and variable light domain (VL) sequences of those depicted in FIGS. 15 18C, 21A-21H and 22. Suitable CD28 ABDs can also include the entire VH and VL sequences as depicted in these sequences and figures, used as scFvs or as Fabs.

In one embodiment, the CD28 antigen binding domain includes the 6 CDRs (i.e., vhCDR1-3 and vlCDR1-3) of any of the CD28 binding domains described herein, including, but not limited to those depicted in FIGS. 1518C and 21A-21H. In some embodiments, the CD28 ABD that binds human CD28 is one of the following CD28 ABDs: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1. 14sp_L1.71sp, CD28.3[CD28]_H0L0, hCD28.3[CD28]_HIL1, 5.11A1[CD28]_H0L0, TGN1412_HIL1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, 9.3[CD28]_HIL1, 9G2[CD28]_H0L0, 9G2[CD28]_HIL1, 2F10A3.140[CD28]_HIL1, and TN228[CD28]_H4L2 (FIGS. 1518C, 21A-21H, and 69A-69C). In exemplary embodiments, the CD28 ABD is CD28 ABDs: 1A7[CD28]_HIL1, 1A7[CD28]_H1.14L1.

In addition to the parental CDR sets disclosed in the figures and sequence listing that form an ABD to CD28, provided herein are variant CD28 ABDS having CDRs that include at least one modification of the CD28 ABD CDRs disclosed herein (e.g., (FIGS. 1518C, and 21A-21H and the sequence listing). In one embodiment, the CD28 ABD of the subject anti-CD28 x anti-PSMA antibody includes a set of 6 CDRs with 1, 2, 3, 4, 5, 6, 7, 8, 9, 10 amino acid modifications as compared to the 6 CDRs of a CD28 ABD as described herein, including the figures and sequence listing. In exemplary embodiments, the CD28 ABD of the subject anti-CD28 x anti-PSMA antibody includes a set of 6 CDRs with 1, 2, 3, 4, 5, 6, 7, 8, 9, 10 amino acid modifications as compared to the 6 CDRs of one of the following CD28 ABDs: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1. 1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_HIL1, 9G2[CD28]_H0L0, 9G2[CD28]_HIL1, 2F10A3.140[CD28]_HIL1, and TN228[CD28]_H4L2 (FIGS. 1518C, 21A-21H, and 69A-69C). In certain embodiments, the CD28 ABD of the subject anti-CD28 x anti-PSMA antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIGS. 1A-1B).

In some embodiments, the CD28 ABD of the subject anti-CD28 x anti-PSMA antibody includes 6 CDRs that are at least 90, 95, 97, 98 or 99% identical to the 6 CDRs of a CD28 ABD as described herein, including the figures and sequence listing. In exemplary embodiments, the CD28 ABD of the subject anti-CD28 x anti-PSMA antibody includes 6 CDRs that are at least 90, 95, 97, 98 or 99% identical to the 6 CDRs of one of the following CD28 ABDs: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_H1L1, 9G2[CD28]_H0L0, 9G2[CD28]_HIL1, 2F10A3.140[CD28]_HIL1, and TN228[CD28]_H4L2 (FIGS. 15-18C, 21A-21H, and 69A-69C). In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIGS. 1A-1B).

In another exemplary embodiment, the CD28 ABD of the subject anti-CD28 x anti-PSMA antibody includes the variable heavy (VH) domain and variable light (VL) domain of any one of the CD28 ABDs described herein, including the figures and sequence listing. In exemplary embodiments, the CD28 ABD is one of the following CD28 ABDs: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_Lisp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_H1L1, 9G2[CD28]_H0L0, 9G2[CD28]_HIL1, 2F10A3.140[CD28]_HIL1, and TN228[CD28]_H4L2 (FIGS. 15-18C, 21A-21H, and 69A-69C). In some embodiments, the CD28 ABD includes a VH/VL pair selected from the VHs and VLs depicted in FIGS. 67, 68, 70A-70F and 71A-71I, respectively.

In some embodiments, the anti-CD28 x anti-PSMA antibody includes a CD28 ABD that includes a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following CD28 ABDs: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_H1L1, 9G2[CD28]_H0L0, 9G2[CD28]_HIL1, 2F10A3.140[CD28]_HIL1, and TN228[CD28]_H4L2 (FIGS. 15-18C, 21A-21H, and 67-69C). In some embodiments, the changes are in a VH domain depicted in FIGS. 15-18C, 21A-21H, and 67-711. In some embodiments, the changes are in a VL domain are depicted in 15-18C, 21A-21H, and 67-711. In some embodiments, the changes are in a VH and VL domain are depicted in FIGS. 15-18C, 21A-21H, and 67-711. In exemplary embodiments, the CD28 ABD is an scFv and the amino acid changes introduce cysteine residues in the VH and/or VL framework regions (FR1-FR4) to allow for “stapling” of the scFv, as described herein. See also WO 2021/030657, which is incorporated by reference in its entirety, including pertinent parts relating to methods for making “stapled” scFvs, and “stapled” scFv compositions. Exemplary “stapled” CD28 binding domains and VH/VL domains that can be included in the subject anti-CD28 x anti-PSMA antibodies depicted herein are included in FIGS. 67-69C. In certain embodiments, the CD28 ABD of the anti-CD28 x anti-PSMA antibody is capable of binding to CD28, as measured at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (sec FIGS. 1A-1B).

In one embodiment, the variant VH and/or VL domain is at least 90, 95, 97, 98 or 99% identical to the VH and/or VL of a CD28 ABD as described herein, including the figures and sequence listing. In exemplary embodiments, the variant VH and/or VL domain is at least 90, 95, 97, 98 or 99% identical to the VH and/or VL of one of the following CD28 ABDs: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_H1L1, 9G2[CD28]_H0L0, 9G2[CD28]_HIL1, 2F10A3.140[CD28]_HIL1, and TN228[CD28]_H4L2 (FIGS. 15-18C, 21A-21H, and 69A-69C). In some embodiments, the CD28 ABD includes a VH that is at least 90, 95, 97, 98 or 99% identical to VH domain depicted in FIGS. 15-18C, 21A-21H, and 67-711. In some embodiments, the CD28 ABD includes a VL that is at least 90, 95, 97, 98 or 99% identical to VL domain depicted in FIGS. 15-18C, 21A-21H, and 67-711. In some embodiments, the CD28 ABD includes a VH and a VL that is at least 90, 95, 97, 98 or 99% identical to a VH domain and a VL domain depicted in FIGS. 15-18C, 21A-21H, and 67-711. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIGS. 1A-1B).

In one embodiment, the CD28 antigen binding domain of the anti-CD28 x anti-PSMA antibody includes a variable heavy domain (VH) having the vhCDR1-3 (i.e., vhCDR1-3) of 1A7_H1 (SEQ ID NO:39, FIG. 18A). In some embodiments, the CD28 antigen binding domain of the anti-CD28 x anti-PSMA antibody further includes any of the CD28 binding domain variable light domains provided herein. In exemplary embodiments, the variable light domain is 1A7_L1.71 (SEQ ID NO:43, FIG. 18A) or a variant thereof. In certain embodiments, the CD28 ABD of the anti-CD28 x anti-PSMA antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD of the anti-CD28 x anti-PSMA antibody is capable of binding human CD28 antigen (see FIGS. 1A-1B). In exemplary embodiments, the anti-CD28 x anti-PSMA antibody is a “1+1 Fab-scFv-Fc,” “2+1 Fab2-scFv-Fc,” or “2+1 mAb-scFv” antibody.

In one embodiment, the CD28 ABD of the anti-CD28 x anti-PSMA antibody includes a variable heavy domain (VH) having vhCDR1-3s with 1, 2, 3, 4, 5, 6, 7, 8, 9, 10 amino acid modifications as compared to the vhCDR1-3 of 1A7_H1 (SEQ ID NO:39, FIG. 18A). In some embodiments, the CD28 antigen binding domain of the anti-CD28 x anti-PSMA antibody further includes any of the CD28 binding domain variable light domains provided herein. In exemplary embodiments, the variable light domain is 1A7_L1.71 (SEQ ID NO:43, FIG. 18A) or a variant thereof. In certain embodiments, the CD28 ABD of the anti-CD28 x anti-PSMA antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD of the anti-CD28 x anti-PSMA antibody is capable of binding human CD28 antigen (see FIGS. 1A-1B). In particular embodiments, the CD28 ABD of the anti-CD28 x anti-PSMA antibody is capable of binding human CD28 antigen (see FIGS. 1A-1B). In exemplary embodiments, the anti-CD28 x anti-PSMA antibody is a “1+1 Fab-scFv-Fc,” “2+1 Fab2-scFv-Fc,” or “2+1 mAb-scFv” antibody.

In some embodiments, the CD28 ABD of the anti-CD28 x anti-PSMA antibody includes a variable heavy domain (VH) having vhCDR1-3s that are at least 90, 95, 97, 98 or 99% identical to the vhCDR1-3 of 1A7_H1 (SEQ ID NO:39, FIG. 18A). In some embodiments, the CD28 antigen binding domain of the anti-CD28 x anti-PSMA antibody further includes any of the CD28 binding domain variable light domains provided herein. In exemplary embodiments, the variable light domain is 1A7_L1.71 (SEQ ID NO:43, FIG. 18A) or a variant thereof. In certain embodiments, the CD28 ABD of the anti-CD28 x anti-PSMA antibody is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD of the anti-CD28 x anti-PSMA antibody is capable of binding human CD28 antigen (see FIGS. 1A-1B). In exemplary embodiments, the anti-CD28 x anti-PSMA antibody is a “1+1 Fab-scFv-Fc,” “2+1 Fab2-scFv-Fc,” or “2+1 mAb-scFv” antibody.

In exemplary embodiments, the CD28 binding domain of the anti-CD28 x anti-PSMA antibody is an scFv. In some embodiments, the anti-CD28 scFv includes one of the CD28 ABD VH and/or VLs described herein or a variant thereof. In some embodiments, the anti-CD28 scFv of the anti-CD28 x anti-PSMA antibody includes one or more “staple” modifications that improves the stability or reduce aggregation of the scFv. In some embodiments, the anti-CD28 x anti-PSMA antibody includes a “stapled” scFv, wherein the stapled scFv includes: a) a first disulfide bond between a structurally conserved surface exposed VH cysteine and a first scFv linker cysteine; b) a second disulfide bond between a structurally conserved surface exposed VL cysteine and a second scFv linker cysteine; or c) the first disulfide bond between the structurally conserved surface exposed VH cysteine and the first scFv linker cysteine and the second disulfide bond between the structurally considered surface exposed VL cysteine and the second scFv linker cysteine. In exemplary embodiments, the “stapled” scFv is a variant of one of the CD28 ABDs described herein, wherein the variant CD28 ABD includes: a) an amino acid substitution to introduce a surface exposed VH cysteine; b) an amino acid substitution to introduce a surface exposed VL cysteine; or c) amino acid substitutions to introduce both a surface exposed VH cysteine and a surface exposed VH cysteine. In some embodiments, the amino acid change(s) is in the VH and/or VL framework regions (FR1, FR2, FR3, and/or FR4). Exemplary scFv “staple linkers” for inclusion in “stapled” scFvs are provided in FIGS. 6A-6B. Methods for making “stapled” scFvs are described, for example, in WO 2021/030657, which is incorporated by reference in its entirety, including pertinent parts relating to methods for making “stapled” scFvs, and “stapled” scFv compositions. In exemplary embodiments, the anti-CD28 x anti-PSMA antibody includes a “stapled” scFv that includes a modified VH and VL of any of the following CD28 ABDs: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_HIL1, 9G2[CD28]_H0L0, 9G2[CD28]_HIL1, 2F10A3.140[CD28]_HIL1, and TN228[CD28]_H4L2 (FIGS. 15-18C, and 21A-21H). Exemplary “stapled” CD28 binding domains include, but are not limited to: 1A7[CD28]_H1sp_L1sp, 1A7[CD28]_H1.1sp_LIsp, 1A7[CD28]_H1sp_L1.71sp, 1A7[CD28]_H1.1sp_L1.71sp, 1A7[CD28]_H1.14sp_L1sp, and 1A7[CD28]_H1.14sp_L1.71sp (FIGS. 70A-70F).

In some embodiments, the anti-CD28 x anti-PSMA antibody includes a CD28 binding domain that includes a VH and VL selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9; and (ii) a VL comprising a vlCDR1, a vlCDR2, and a vICDR3 having an amino acid sequence of a vlCDR1, a vlCDR2, and a vlCDR3, respectively, of a VL having an amino acid sequence of SEQ ID NO: 13; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:31; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:35; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:43; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:47; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:51; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:55; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:59; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:67; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:90; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:94; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:98; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 102; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 106; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:110; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:114; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 118; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:122; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 126; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:130; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:134; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:138; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 142; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 146; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:150; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 158; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 166; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:170; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:174; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 178; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:182; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:186; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 190; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 194; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 198; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:202; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:380; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:384; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:396; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:400; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:404; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:408; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:412; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:416; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:420; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:424.

In some embodiments, the anti-CD28 x anti-PSMA antibody includes a CD28 binding domain that includes a VH and VL selected from the following:
(i) a VH comprising a vhCDR1 having an amino acid sequence of SEQ ID NO:10; a vhCDR2 having an amino acid sequence of SEQ ID NO:11; and a vhCDR3 having an amino acid sequence of SEQ ID NO:12; and (ii) a VL comprising a vlCDR1 having an amino acid sequence of SEQ ID NO:14, a vlCDR2 having an amino acid sequence of SEQ ID NO: 15, and a vlCDR3 having an amino acid sequence of SEQ ID NO: 16; or
(i) a VH comprising a vhCDR1 having an amino acid sequence of SEQ ID NO:32; a vhCDR2 having an amino acid sequence of SEQ ID NO:33; and a vhCDR3 having an amino acid sequence of SEQ ID NO:34; and (ii) a VL comprising a vlCDR1 having an amino acid sequence of SEQ ID NO:36, a vlCDR2 having an amino acid sequence of SEQ ID NO: 37, and a vlCDR3 having an amino acid sequence of SEQ ID NO:38; or
i) a VH comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; or
i) a VH comprising a vhCDR1 having an amino acid sequence of SEQ ID NO:48; a vhCDR2 having an amino acid sequence of SEQ ID NO:49; and a vhCDR3 having an amino acid sequence of SEQ ID NO:50; and (ii) a VL comprising a vlCDR1 having an amino acid sequence of SEQ ID NO:52, a vlCDR2 having an amino acid sequence of SEQ ID NO: 53, and a vlCDR3 having an amino acid sequence of SEQ ID NO:54; or
i) a VH comprising a vhCDR1 having an amino acid sequence of SEQ ID NO:56; a vhCDR2 having an amino acid sequence of SEQ ID NO:57; and a vhCDR3 having an amino acid sequence of SEQ ID NO:58; and (ii) a VL comprising a vlCDR1 having an amino acid sequence of SEQ ID NO:60, a vlCDR2 having an amino acid sequence of SEQ ID NO:61, and a vlCDR3 having an amino acid sequence of SEQ ID NO:62; or
i) a VH comprising a vhCDR1 having an amino acid sequence of SEQ ID NO:64; a vhCDR2 having an amino acid sequence of SEQ ID NO:65; and a vhCDR3 having an amino acid sequence of SEQ ID NO:66; and (ii) a VL comprising a vlCDR1 having an amino acid sequence of SEQ ID NO:68, a vlCDR2 having an amino acid sequence of SEQ ID NO:69, and a vlCDR3 having an amino acid sequence of SEQ ID NO:70; or
i) a VH comprising a vhCDR1 having an amino acid sequence of SEQ ID NO:87; a vhCDR2 having an amino acid sequence of SEQ ID NO:88; and a vhCDR3 having an amino acid sequence of SEQ ID NO:89; and (ii) a VL comprising a vlCDR1 having an amino acid sequence of SEQ ID NO:91, a vlCDR2 having an amino acid sequence of SEQ ID NO:92, and a vlCDR3 having an amino acid sequence of SEQ ID NO:93; or
i) a VH comprising a vhCDR1 having an amino acid sequence of SEQ ID NO:95; a vhCDR2 having an amino acid sequence of SEQ ID NO:96; and a vhCDR3 having an amino acid sequence of SEQ ID NO:97; and (ii) a VL comprising a vlCDR1 having an amino acid sequence of SEQ ID NO:99, a vlCDR2 having an amino acid sequence of SEQ ID NO:100, and a vlCDR3 having an amino acid sequence of SEQ ID NO: 101; or
i) a VH comprising a vhCDR1 having an amino acid sequence of SEQ ID NO:103; a vhCDR2 having an amino acid sequence of SEQ ID NO:104; and a vhCDR3 having an amino acid sequence of SEQ ID NO:105; and (ii) a VL comprising a vlCDR1 having an amino acid sequence of SEQ ID NO: 107, a vlCDR2 having an amino acid sequence of SEQ ID NO:108, and a vlCDR3 having an amino acid sequence of SEQ ID NO:109; or
i) a VH comprising a vhCDR1 having an amino acid sequence of SEQ ID NO:111; a vhCDR2 having an amino acid sequence of SEQ ID NO:112; and a vhCDR3 having an amino acid sequence of SEQ ID NO:113; and (ii) a VL comprising a vlCDR1 having an amino acid sequence of SEQ ID NO: 115, a vlCDR2 having an amino acid sequence of SEQ ID NO: 116, and a vlCDR3 having an amino acid sequence of SEQ ID NO:117; or
i) a VH comprising a vhCDR1 having an amino acid sequence of SEQ ID NO:119; a vhCDR2 having an amino acid sequence of SEQ ID NO:120; and a vhCDR3 having an amino acid sequence of SEQ ID NO: 121; and (ii) a VL comprising a vlCDR1 having an amino acid sequence of SEQ ID NO:123, a vlCDR2 having an amino acid sequence of SEQ ID NO: 124, and a vlCDR3 having an amino acid sequence of SEQ ID NO:125; or
i) a VH comprising a vhCDR1 having an amino acid sequence of SEQ ID NO:127; a vhCDR2 having an amino acid sequence of SEQ ID NO:128; and a vhCDR3 having an amino acid sequence of SEQ ID NO: 129; and (ii) a VL comprising a vlCDR1 having an amino acid sequence of SEQ ID NO: 131, a vlCDR2 having an amino acid sequence of SEQ ID NO:132, and a vlCDR3 having an amino acid sequence of SEQ ID NO:133; or
i) a VH comprising a vhCDR1 having an amino acid sequence of SEQ ID NO:135; a vhCDR2 having an amino acid sequence of SEQ ID NO:136; and a vhCDR3 having an amino acid sequence of SEQ ID NO: 137; and (ii) a VL comprising a vlCDR1 having an amino acid sequence of SEQ ID NO:139, a vlCDR2 having an amino acid sequence of SEQ ID NO:140, and a vlCDR3 having an amino acid sequence of SEQ ID NO:141; or
i) a VH comprising a vhCDR1 having an amino acid sequence of SEQ ID NO:143; a vhCDR2 having an amino acid sequence of SEQ ID NO:144; and a vhCDR3 having an amino acid sequence of SEQ ID NO: 115; and (ii) a VL comprising a vlCDR1 having an amino acid sequence of SEQ ID NO: 147, a vlCDR2 having an amino acid sequence of SEQ ID NO: 149, and a vlCDR3 having an amino acid sequence of SEQ ID NO:149; or
i) a VH comprising a vhCDR1 having an amino acid sequence of SEQ ID NO:151; a vhCDR2 having an amino acid sequence of SEQ ID NO:152; and a vhCDR3 having an amino acid sequence of SEQ ID NO:153; and (ii) a VL comprising a vlCDR1 having an amino acid sequence of SEQ ID NO: 155, a vlCDR2 having an amino acid sequence of SEQ ID NO:156, and a vlCDR3 having an amino acid sequence of SEQ ID NO:157; or
i) a VH comprising a vhCDR1 having an amino acid sequence of SEQ ID NO:159; a vhCDR2 having an amino acid sequence of SEQ ID NO:160; and a vhCDR3 having an amino acid sequence of SEQ ID NO: 161; and (ii) a VL comprising a vlCDR1 having an amino acid sequence of SEQ ID NO:163, a vlCDR2 having an amino acid sequence of SEQ ID NO:164, and a vlCDR3 having an amino acid sequence of SEQ ID NO:165; or
i) a VH comprising a vhCDR1 having an amino acid sequence of SEQ ID NO:167; a vhCDR2 having an amino acid sequence of SEQ ID NO:168; and a vhCDR3 having an amino acid sequence of SEQ ID NO: 169; and (ii) a VL comprising a vlCDR1 having an amino acid sequence of SEQ ID NO: 171, a vlCDR2 having an amino acid sequence of SEQ ID NO: 172, and a vlCDR3 having an amino acid sequence of SEQ ID NO:173; or
i) a VH comprising a vhCDR1 having an amino acid sequence of SEQ ID NO:175; a vhCDR2 having an amino acid sequence of SEQ ID NO:176; and a vhCDR3 having an amino acid sequence of SEQ ID NO:177; and (ii) a VL comprising a vlCDR1 having an amino acid sequence of SEQ ID NO: 179, a vlCDR2 having an amino acid sequence of SEQ ID NO:180, and a vlCDR3 having an amino acid sequence of SEQ ID NO:181; or
i) a VH comprising a vhCDR1 having an amino acid sequence of SEQ ID NO:183; a vhCDR2 having an amino acid sequence of SEQ ID NO:184; and a vhCDR3 having an amino acid sequence of SEQ ID NO: 185; and (ii) a VL comprising a vlCDR1 having an amino acid sequence of SEQ ID NO:187, a vlCDR2 having an amino acid sequence of SEQ ID NO:188, and a vlCDR3 having an amino acid sequence of SEQ ID NO:189; or
i) a VH comprising a vhCDR1 having an amino acid sequence of SEQ ID NO:191; a vhCDR2 having an amino acid sequence of SEQ ID NO:192; and a vhCDR3 having an amino acid sequence of SEQ ID NO: 193; and (ii) a VL comprising a vlCDR1 having an amino acid sequence of SEQ ID NO: 195, a vlCDR2 having an amino acid sequence of SEQ ID NO: 196, and a vlCDR3 having an amino acid sequence of SEQ ID NO:197; or
i) a VH comprising a vhCDR1 having an amino acid sequence of SEQ ID NO:199; a vhCDR2 having an amino acid sequence of SEQ ID NO:200; and a vhCDR3 having an amino acid sequence of SEQ ID NO:201; and (ii) a VL comprising a vlCDR1 having an amino acid sequence of SEQ ID NO:203, a vlCDR2 having an amino acid sequence of SEQ ID NO:204, and a vlCDR3 having an amino acid sequence of SEQ ID NO:205; or
i) a VH comprising a vhCDR1 having an amino acid sequence of SEQ ID NO:381; a vhCDR2 having an amino acid sequence of SEQ ID NO:382; and a vhCDR3 having an amino acid sequence of SEQ ID NO:383; and (ii) a VL comprising a vlCDR1 having an amino acid sequence of SEQ ID NO:385, a vlCDR2 having an amino acid sequence of SEQ ID NO:386, and a vlCDR3 having an amino acid sequence of SEQ ID NO:387; or
i) a VH comprising a vhCDR1 having an amino acid sequence of SEQ ID NO:389; a vhCDR2 having an amino acid sequence of SEQ ID NO:390; and a vhCDR3 having an amino acid sequence of SEQ ID NO:391; and (ii) a VL comprising a vlCDR1 having an amino acid sequence of SEQ ID NO:393, a vlCDR2 having an amino acid sequence of SEQ ID NO:394, and a vlCDR3 having an amino acid sequence of SEQ ID NO:395; or
i) a VH comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403; or
i) a VH comprising a vhCDR1 having an amino acid sequence of SEQ ID NO:405; a vhCDR2 having an amino acid sequence of SEQ ID NO:406; and a vhCDR3 having an amino acid sequence of SEQ ID NO:407; and (ii) a VL comprising a vlCDR1 having an amino acid sequence of SEQ ID NO:409, a vlCDR2 having an amino acid sequence of SEQ ID NO:410, and a vlCDR3 having an amino acid sequence of SEQ ID NO:411; or
i) a VH comprising a vhCDR1 having an amino acid sequence of SEQ ID NO:413; a vhCDR2 having an amino acid sequence of SEQ ID NO:414; and a vhCDR3 having an amino acid sequence of SEQ ID NO:415; and (ii) a VL comprising a vlCDR1 having an amino acid sequence of SEQ ID NO:417, a vlCDR2 having an amino acid sequence of SEQ ID NO:418, and a vlCDR3 having an amino acid sequence of SEQ ID NO:419; or
i) a VH comprising a vhCDR1 having an amino acid sequence of SEQ ID NO:421; a vhCDR2 having an amino acid sequence of SEQ ID NO:422; and a vhCDR3 having an amino acid sequence of SEQ ID NO:423; and (ii) a VL comprising a vlCDR1 having an amino acid sequence of SEQ ID NO:425, a vlCDR2 having an amino acid sequence of SEQ ID NO:426, and a vlCDR3 having an amino acid sequence of SEQ ID NO:427.

In some embodiments, the anti-CD28 x anti-PSMA antibody includes a CD28 binding domain that includes a VH and VL selected from the following:
(i) a VH having an amino acid sequence that is at least about 95% identical to SEQ ID NO:9; and (ii) a VL having an amino acid sequence that is at least about 95% identical to SEQ ID NO:13; or
(i) a VH having an amino acid sequence that is at least about 95% identical to SEQ ID NO:31; and (ii) a VL having an amino acid sequence that is at least about 95% identical to SEQ ID NO:35; or
(i) a VH having an amino acid sequence that is at least about 95% identical to SEQ ID NO:39; and (ii) a VL having an amino acid sequence that is at least about 95% identical to SEQ ID NO:43; or
(i) a VH having an amino acid sequence that is at least about 95% identical to SEQ ID NO:47; and (ii) a VL having an amino acid sequence that is at least about 95% identical to SEQ ID NO:51; or
(i) a VH having an amino acid sequence that is at least about 95% identical to SEQ ID NO:55; and (ii) a VL having an amino acid sequence that is at least about 95% identical to SEQ ID NO:59; or
(i) a VH having an amino acid sequence that is at least about 95% identical to SEQ ID NO:63; and (ii) a VL having an amino acid sequence that is at least about 95% identical to SEQ ID NO:67; or
(i) a VH having an amino acid sequence that is at least about 95% identical to SEQ ID NO:86; and (ii) a VL having an amino acid sequence that is at least about 95% identical to SEQ ID NO:90; or
(i) a VH having an amino acid sequence that is at least about 95% identical to SEQ ID NO:94; and (ii) a VL having an amino acid sequence that is at least about 95% identical to SEQ ID NO:98; or
(i) a VH having an amino acid sequence that is at least about 95% identical to SEQ ID NO:102; and (ii) a VL having an amino acid sequence that is at least about 95% identical to SEQ ID NO: 106; or
(i) a VH having an amino acid sequence that is at least about 95% identical to SEQ ID NO:110; and (ii) a VL having an amino acid sequence that is at least about 95% identical to SEQ ID NO:114; or
(i) a VH having an amino acid sequence that is at least about 95% identical to SEQ ID NO:118; and (ii) a VL having an amino acid sequence that is at least about 95% identical to SEQ ID NO:122; or
(i) a VH having an amino acid sequence that is at least about 95% identical to SEQ ID NO:126; and (ii) a VL having an amino acid sequence that is at least about 95% identical to SEQ ID NO:130; or
(i) a VH having an amino acid sequence that is at least about 95% identical to SEQ ID NO:134; and (ii) a VL having an amino acid sequence that is at least about 95% identical to SEQ ID NO:138; or
(i) a VH having an amino acid sequence that is at least about 95% identical to SEQ ID NO:142; and (ii) a VL having an amino acid sequence that is at least about 95% identical to SEQ ID NO:146; or
(i) a VH having an amino acid sequence that is at least about 95% identical to SEQ ID NO:150; and (ii) a VL having an amino acid sequence that is at least about 95% identical to SEQ ID NO:154; or
(i) a VH having an amino acid sequence that is at least about 95% identical to SEQ ID NO:158; and (ii) a VL having an amino acid sequence that is at least about 95% identical to SEQ ID NO:162; or
(i) a VH having an amino acid sequence that is at least about 95% identical to SEQ ID NO:166; and (ii) a VL having an amino acid sequence that is at least about 95% identical to SEQ ID NO:170; or
(i) a VH having an amino acid sequence that is at least about 95% identical to SEQ ID NO:174; and (ii) a VL having an amino acid sequence that is at least about 95% identical to SEQ ID NO:178; or
(i) a VH having an amino acid sequence that is at least about 95% identical to SEQ ID NO:182; and (ii) a VL having an amino acid sequence that is at least about 95% identical to SEQ ID NO:186; or
(i) a VH having an amino acid sequence that is at least about 95% identical to SEQ ID NO:190; and (ii) a VL having an amino acid sequence that is at least about 95% identical to SEQ ID NO:194; or
(i) a VH having an amino acid sequence that is at least about 95% identical to SEQ ID NO:198; and (ii) a VL having an amino acid sequence that is at least about 95% identical to SEQ ID NO:202; or
(i) a VH having an amino acid sequence that is at least about 95% identical to SEQ ID NO:380; and (ii) a VL having an amino acid sequence that is at least about 95% identical to SEQ ID NO:384; or
(i) a VH having an amino acid sequence that is at least about 95% identical to SEQ ID NO:388; and (ii) a VL having an amino acid sequence that is at least about 95% identical to SEQ ID NO:392; or
(i) a VH having an amino acid sequence that is at least about 95% identical to SEQ ID NO:396; and (ii) a VL having an amino acid sequence that is at least about 95% identical to SEQ ID NO:400; or
(i) a VH having an amino acid sequence that is at least about 95% identical to SEQ ID NO:404; and (ii) a VL having an amino acid sequence that is at least about 95% identical to SEQ ID NO:408; or
(i) a VH having an amino acid sequence that is at least about 95% identical to SEQ ID NO:412; and (ii) a VL having an amino acid sequence that is at least about 95% identical to SEQ ID NO:416; or
(i) a VH having an amino acid sequence that is at least about 95% identical to SEQ ID NO:420; and (ii) a VL having an amino acid sequence that is at least about 95% identical to SEQ ID NO:424.

In some embodiments, the anti-CD28 x anti-PSMA antibody includes a CD28 binding domain that includes a VH and VL selected from the following:
(i) a VH having an amino acid sequence of SEQ ID NO:9; and (ii) a VL having an amino acid sequence of SEQ ID NO: 13; or
(i) a VH having an amino acid sequence of SEQ ID NO:31; and (ii) a VL having an amino acid sequence of SEQ ID NO:35; or
(i) a VH having an amino acid sequence of SEQ ID NO:39; and (ii) a VL having an amino acid sequence of SEQ ID NO:43; or
(i) a VH having an amino acid sequence of SEQ ID NO:47; and (ii) a VL having an amino acid sequence of SEQ ID NO:51; or
(i) a VH having an amino acid sequence of SEQ ID NO:55; and (ii) a VL having an amino acid sequence of SEQ ID NO:59; or
(i) a VH having an amino acid sequence of SEQ ID NO:63; and (ii) a VL having an amino acid sequence of SEQ ID NO:67; or
(i) a VH having an amino acid sequence of SEQ ID NO:86; and (ii) a VL having an amino acid sequence of SEQ ID NO:90; or
(i) a VH having an amino acid sequence of SEQ ID NO:94; and (ii) a VL having an amino acid sequence of SEQ ID NO:98; or
(i) a VH having an amino acid sequence of SEQ ID NO:102; and (ii) a VL having an amino acid sequence of SEQ ID NO: 106; or
(i) a VH having an amino acid sequence of SEQ ID NO:110; and (ii) a VL having an amino acid sequence of SEQ ID NO: 114; or
(i) a VH having an amino acid sequence of SEQ ID NO:118; and (ii) a VL having an amino acid sequence of SEQ ID NO:122; or
(i) a VH having an amino acid sequence of SEQ ID NO:126; and (ii) a VL having an amino acid sequence of SEQ ID NO:130; or
(i) a VH having an amino acid sequence of SEQ ID NO:134; and (ii) a VL having an amino acid sequence of SEQ ID NO:138; or
(i) a VH having an amino acid sequence of SEQ ID NO:142; and (ii) a VL having an amino acid sequence of SEQ ID NO: 146; or
(i) a VH having an amino acid sequence of SEQ ID NO:150; and (ii) a VL having an amino acid sequence of SEQ ID NO:154; or
(i) a VH having an amino acid sequence of SEQ ID NO:158; and (ii) a VL having an amino acid sequence of SEQ ID NO:162; or
(i) a VH having an amino acid sequence of SEQ ID NO:166; and (ii) a VL having an amino acid sequence of SEQ ID NO:170; or
(i) a VH having an amino acid sequence of SEQ ID NO: 174; and (ii) a VL having an amino acid sequence of SEQ ID NO: 178; or
(i) a VH having an amino acid sequence of SEQ ID NO:182; and (ii) a VL having an amino acid sequence of SEQ ID NO:186; or
(i) a VH having an amino acid sequence of SEQ ID NO:190; and (ii) a VL having an amino acid sequence of SEQ ID NO: 194; or
(i) a VH having an amino acid sequence of SEQ ID NO:198; and (ii) a VL having an amino acid sequence of SEQ ID NO:202; or
(i) a VH having an amino acid sequence of SEQ ID NO:380; and (ii) a VL having an amino acid sequence of SEQ ID NO:384; or
(i) a VH having an amino acid sequence of SEQ ID NO:388; and (ii) a VL having an amino acid sequence of SEQ ID NO:392; or
(i) a VH having an amino acid sequence of SEQ ID NO:396; and (ii) a VL having an amino acid sequence of SEQ ID NO:400; or
(i) a VH having an amino acid sequence of SEQ ID NO:404; and (ii) a VL having an amino acid sequence of SEQ ID NO:408; or
(i) a VH having an amino acid sequence of SEQ ID NO:412; and (ii) a VL having an amino acid sequence of SEQ ID NO:416; or
(i) a VH having an amino acid sequence of SEQ ID NO:420; and (ii) a VL having an amino acid sequence of SEQ ID NO:424.

In some embodiments, the CD28 binding domain included in the anti-CD28 x anti-PSMA antibody includes a vhCDR1 having an amino acid of SEQ ID NO:72, a vhCDR2 having an amino acid sequence of SEQ ID NO:74, and/or a vhCDR3 having an amino acid sequence of SEQ ID NO: 76. In some embodiments, the CD28 binding domain included in the anti-CD28 x anti-PSMA antibody includes a vlCDR1 having an amino acid of SEQ ID NO:80, a vlCDR2 having an amino acid sequence of SEQ ID NO:82, and/or a vlCDR3 having an amino acid sequence of SEQ ID NO: 84.

### B. PSMA Binding Domains

The anti-CD28 x anti-PSMA antibodies provided herein include at least one PSMA binding domain. Subject antibodies that include such PSMA antigen binding domains (e.g., anti-PSMA x anti-CD3 bispecific antibodies) advantageously target cells that express high levels of PSMA over those that express levels of PSMA (e.g., normal cells).

As will be appreciated by those in the art, suitable PSMA binding domains can comprise a set of 6 CDRs as depicted in the sequence listing and FIGS. 25A-25E and 26A-26B, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 25A-25E and 26A-26B and the sequence listing (see Table 2). Suitable PSMA ABDs can also include the entire VH and VL sequences as depicted in these sequences and figures, used as scFvs or as Fab domains.

In one embodiment, the PSMA antigen binding domain of the anti-CD28 x anti-PSMA antibody includes the 6 CDRs (i.e., vhCDR1-3 and vlCDR1-3) of a PSMA ABD described herein, including the Figures and sequence listing. In exemplary embodiments, the PSMA ABD is one of the following PSMA ABDs: A10[PSMA], A10v2[PSMA], DO1[PSMA], DO1v2[PSMA], E07[PSMA], F02[PSMA], A11[PSMA], F07[PSMA], F07v2[PSMA], G02[PSMA], F01[PSMA], F01v2[PSMA], 011A11[PSMA], PSMB896[PSMA], PSMA-H[PSMA], and D7[PSMA] (FIGS. 25A-25E and 26A-26B).

In addition to the parental CDR sets disclosed in the figures and sequence listing that form an ABD to PSMA, provided herein are variant PSMA ABDS having CDRs that include at least one modification of the PSMA ABD CDRs disclosed herein. In one embodiment, the PSMA ABD includes a set of 6 CDRs with 1, 2, 3, 4, 5, 6, 7, 8, 9, 10 amino acid modifications as compared to the 6 CDRs of a PSMA ABD described herein, including the figures and sequence listing. In exemplary embodiments, the PSMA ABD of the anti-CD28 x anti-PSMA antibody includes a set of 6 CDRs with 1, 2, 3, 4, 5, 6, 7, 8, 9, 10 amino acid modifications as compared to the 6 CDRs of one of the following PSMA ABDs: A10[PSMA], A10v2[PSMA], DO1[PSMA], DO1v2[PSMA], E07[PSMA], F02[PSMA], A11[PSMA], F07[PSMA], F07v2[PSMA], G02[PSMA], F01[PSMA], F01v2[PSMA], 011A11[PSMA], PSMB896[PSMA], PSMA-H[PSMA], and D7[PSMA] (FIGS. 25A-25E and 26A-26B). In certain embodiments, the variant PSMA ABD is capable of binding PSMA antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PSMA ABD is capable of binding human PSMA antigen (see Example 5).

In one embodiment, the PSMA ABD of the anti-CD28 x anti-PSMA antibody includes 6 CDRs that are at least 90, 95, 97, 98 or 99% identical to the 6 CDRs of a PSMA ABD as described herein, including the figures and sequence listing. In exemplary embodiments, the PSMA ABD includes 6 CDRs that are at least 90, 95, 97, 98 or 99% identical to the 6 CDRs of one of the following PSMA ABDs: A10[PSMA], A10v2[PSMA], DO1[PSMA], DO1v2[PSMA], E07[PSMA], F02[PSMA], A11[PSMA], F07[PSMA], F07v2[PSMA], G02[PSMA], FOI[PSMA], F01v2[PSMA], 011A11[PSMA], PSMB896[PSMA], PSMA-H[PSMA], and D7[PSMA] (FIGS. 25A-25E and 26A-26B). In certain embodiments, the PSMA ABD is capable of binding to PSMA antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PSMA ABD is capable of binding human PSMA antigen (see FIGS. 2A-2B).

In another exemplary embodiment, the PSMA ABD of the anti-CD28 x anti-PSMA antibody include the variable heavy (VH) domain and variable light (VL) domain of any one of the PSMA ABDs described herein, including the figures and sequence listing. In exemplary embodiments, the PSMA ABD is one of the following PSMA ABDs: A10[PSMA], A10v2[PSMA], DO1[PSMA], DO1v2[PSMA], E07[PSMA], F02[PSMA], All[PSMA], F07[PSMA], F07v2[PSMA], G02[PSMA], FOI[PSMA], F01v2[PSMA], 011A11[PSMA], PSMB896[PSMA], PSMA-H[PSMA], and D7[PSMA] (FIGS. 25A-25E and 26A-26B).

In addition to the parental PSMA variable heavy and variable light domains disclosed herein, provided herein are PSMA ABDs that include a variable heavy domain and/or a variable light domain that are variants of a PSMA ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a PSMA ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following PSMA ABDs: A10[PSMA], A10v2[PSMA], DO1[PSMA], DO1v2[PSMA], E07[PSMA], F02[PSMA], A11[PSMA], F07[PSMA], F07v2[PSMA], G02[PSMA], F01[PSMA], F01v2[PSMA], 011A11[PSMA], PSMB896[PSMA], PSMA-H[PSMA], and D7[PSMA] (FIGS. 25A-25E and 26A-26B). In some embodiments, the changes are in a VH domain depicted in FIGS. 25A-25E and 26A-26B. In some embodiments, the changes are in a VL domain are depicted in FIGS. 25A-25E and 26A-26B. In some embodiments, the changes are in a VH and VL domain are depicted in FIGS. 25A-25E and 26A-26B. In some embodiments, the amino acid change(s) is in the VH and/or VL framework regions (FR1, FR2, FR3, and/or FR4). In certain embodiments, the PSMA ABD of the anti-CD28 x anti-PSMA antibody is capable of binding to PSMA, as measured at least one of a Biacore, surface plasmon resonance (SPR), flow cytometry, and/or BLI (biolayer interferometry, e.g., Octet assay) assay, with the latter finding particular use in many embodiments. In particular embodiments, the PSMA ABD is capable of binding human PSMA antigen.

In one embodiment, the variant VH and/or VL domain is at least 90, 95, 97, 98 or 99% identical to the VH and/or VL of a PSMA ABD as described herein, including the figures and sequence listing. In exemplary embodiments, the variant VH and/or VL domain is at least 90, 95, 97, 98 or 99% identical to the VH and/or VL of one of the following PSMA ABDs: A10[PSMA], A10v2[PSMA], DO1[PSMA], DO1v2[PSMA], E07[PSMA], F02[PSMA], A11[PSMA], F07[PSMA], F07v2[PSMA], G02[PSMA], F01[PSMA], F01v2[PSMA], 011A11[PSMA], PSMB896[PSMA], PSMA-H[PSMA], and D7[PSMA] (FIGS. 25A-25E and 26A-26B). In some embodiments, the PSMA ABD includes a VH that is at least 90, 95, 97, 98 or 99% identical to VH domain depicted in FIGS. 25A-25E and 26A-26B. In some embodiments, the CD28 PSMA includes a VL that is at least 90, 95, 97, 98 or 99% identical to VL domain depicted in FIGS. 25A-25E and 26A-26B. In some embodiments, the PSMA ABD includes a VH and a VL that is at least 90, 95, 97, 98 or 99% identical to a VH and a VL domains depicted in FIGS. 25A-25E and 26A-26B. In certain embodiments, the PSMA ABD of the anti-CD28 x anti-PSMA antibody is capable of binding to the PSMA, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PSMA ABD is capable of binding human PSMA antigen.

In some embodiments, the anti-CD28 x anti-PSMA antibody is a bivalent antibody (e.g., 1+1 Fab-scFv-Fc format antibody) that includes one PSMA binding domain. In other embodiments, the anti-CD28 x anti-PSMA antibody is a trivalent antibody (e.g., 2+1 Fab2-scFv-Fc format antibody) that includes two PSMA binding domains.

In some embodiments, the VH and VL of the PSMA binding domain(s) are selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:206; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:210; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:214; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:218; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:222; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:226; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:230; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:234; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:238; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:242; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:246; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:250; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:254; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:258; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:262; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:266; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:270; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:274; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:278; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:282; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:286; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:290; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:294; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:298; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:302; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:306; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:310; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:314; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:318; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:322; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:326; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:330; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:334; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:338.

In some embodiments, the VH and VL of the PSMA binding domain(s) are selected from the following:
(i) a VH comprising a vhCDR1 having an amino acid sequence of SEQ ID NO:207; a vhCDR2 having an amino acid sequence of SEQ ID NO:208; and a vhCDR3 having an amino acid sequence of SEQ ID NO:209; and (ii) a VL comprising a vlCDR1 having an amino acid sequence of SEQ ID NO:211, a vlCDR2 having an amino acid sequence of SEQ ID NO: 212, and a vlCDR3 having an amino acid sequence of SEQ ID NO:213; or
(i) a VH comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221;
(i) a VH comprising a vhCDR1 having an amino acid sequence of SEQ ID NO:223; a vhCDR2 having an amino acid sequence of SEQ ID NO:224; and a vhCDR3 having an amino acid sequence of SEQ ID NO:225; and (ii) a VL comprising a vlCDR1 having an amino acid sequence of SEQ ID NO:227, a vlCDR2 having an amino acid sequence of SEQ ID NO: 228, and a vlCDR3 having an amino acid sequence of SEQ ID NO:229; or
(i) a VH comprising a vhCDR1 having an amino acid sequence of SEQ ID NO:231; a vhCDR2 having an amino acid sequence of SEQ ID NO:232; and a vhCDR3 having an amino acid sequence of SEQ ID NO:233; and (ii) a VL comprising a vlCDR1 having an amino acid sequence of SEQ ID NO:235, a vlCDR2 having an amino acid sequence of SEQ ID NO: 236, and a vlCDR3 having an amino acid sequence of SEQ ID NO:237; or
(i) a VH comprising a vhCDR1 having an amino acid sequence of SEQ ID NO:239; a vhCDR2 having an amino acid sequence of SEQ ID NO:240; and a vhCDR3 having an amino acid sequence of SEQ ID NO:241; and (ii) a VL comprising a vlCDR1 having an amino acid sequence of SEQ ID NO:243, a vlCDR2 having an amino acid sequence of SEQ ID NO: 244, and a vlCDR3 having an amino acid sequence of SEQ ID NO:245; or
(i) a VH comprising a vhCDR1 having an amino acid sequence of SEQ ID NO:247; a vhCDR2 having an amino acid sequence of SEQ ID NO:248; and a vhCDR3 having an amino acid sequence of SEQ ID NO:249; and (ii) a VL comprising a vlCDR1 having an amino acid sequence of SEQ ID NO:251, a vlCDR2 having an amino acid sequence of SEQ ID NO: 252, and a vlCDR3 having an amino acid sequence of SEQ ID NO:253; or
(i) a VH comprising a vhCDR1 having an amino acid sequence of SEQ ID NO:255; a vhCDR2 having an amino acid sequence of SEQ ID NO:256; and a vhCDR3 having an amino acid sequence of SEQ ID NO:257; and (ii) a VL comprising a vlCDR1 having an amino acid sequence of SEQ ID NO:259, a vlCDR2 having an amino acid sequence of SEQ ID NO: 260, and a vlCDR3 having an amino acid sequence of SEQ ID NO:261; or
(i) a VH comprising a vhCDR1 having an amino acid sequence of SEQ ID NO:263; a vhCDR2 having an amino acid sequence of SEQ ID NO:264; and a vhCDR3 having an amino acid sequence of SEQ ID NO:265; and (ii) a VL comprising a vlCDR1 having an amino acid sequence of SEQ ID NO:267, a vlCDR2 having an amino acid sequence of SEQ ID NO: 268, and a vlCDR3 having an amino acid sequence of SEQ ID NO:269; or
(i) a VH comprising a vhCDR1 having an amino acid sequence of SEQ ID NO:271; a vhCDR2 having an amino acid sequence of SEQ ID NO:212; and a vhCDR3 having an amino acid sequence of SEQ ID NO:273; and (ii) a VL comprising a vlCDR1 having an amino acid sequence of SEQ ID NO:275, a vlCDR2 having an amino acid sequence of SEQ ID NO: 276, and a vlCDR3 having an amino acid sequence of SEQ ID NO:277; or
(i) a VH comprising a vhCDR1 having an amino acid sequence of SEQ ID NO:279; a vhCDR2 having an amino acid sequence of SEQ ID NO:280; and a vhCDR3 having an amino acid sequence of SEQ ID NO:281; and (ii) a VL comprising a vlCDR1 having an amino acid sequence of SEQ ID NO:283, a vlCDR2 having an amino acid sequence of SEQ ID NO: 284, and a vlCDR3 having an amino acid sequence of SEQ ID NO:285; or
(i) a VH comprising a vhCDR1 having an amino acid sequence of SEQ ID NO:287; a vhCDR2 having an amino acid sequence of SEQ ID NO:288; and a vhCDR3 having an amino acid sequence of SEQ ID NO:289; and (ii) a VL comprising a vlCDR1 having an amino acid sequence of SEQ ID NO:291, a vlCDR2 having an amino acid sequence of SEQ ID NO: 292, and a vlCDR3 having an amino acid sequence of SEQ ID NO:293; or
(i) a VH comprising a vhCDR1 having an amino acid sequence of SEQ ID NO:295; a vhCDR2 having an amino acid sequence of SEQ ID NO:296; and a vhCDR3 having an amino acid sequence of SEQ ID NO:297; and (ii) a VL comprising a vlCDR1 having an amino acid sequence of SEQ ID NO:299, a vlCDR2 having an amino acid sequence of SEQ ID NO: 300, and a vlCDR3 having an amino acid sequence of SEQ ID NO:301; or
(i) a VH comprising a vhCDR1 having an amino acid sequence of SEQ ID NO:303; a vhCDR2 having an amino acid sequence of SEQ ID NO:304; and a vhCDR3 having an amino acid sequence of SEQ ID NO:305; and (ii) a VL comprising a vlCDR1 having an amino acid sequence of SEQ ID NO:307, a vlCDR2 having an amino acid sequence of SEQ ID NO: 308, and a vlCDR3 having an amino acid sequence of SEQ ID NO:309; or
(i) a VH comprising a vhCDR1 having an amino acid sequence of SEQ ID NO:311; a vhCDR2 having an amino acid sequence of SEQ ID NO:312; and a vhCDR3 having an amino acid sequence of SEQ ID NO:313; and (ii) a VL comprising a vlCDR1 having an amino acid sequence of SEQ ID NO:315, a vlCDR2 having an amino acid sequence of SEQ ID NO: 316, and a vlCDR3 having an amino acid sequence of SEQ ID NO:317; or
(i) a VH comprising a vhCDR1 having an amino acid sequence of SEQ ID NO:319; a vhCDR2 having an amino acid sequence of SEQ ID NO:320; and a vhCDR3 having an amino acid sequence of SEQ ID NO:321; and (ii) a VL comprising a vlCDR1 having an amino acid sequence of SEQ ID NO:323, a vlCDR2 having an amino acid sequence of SEQ ID NO: 324, and a vlCDR3 having an amino acid sequence of SEQ ID NO:325; or
(i) a VH comprising a vhCDR1 having an amino acid sequence of SEQ ID NO:327; a vhCDR2 having an amino acid sequence of SEQ ID NO:328; and a vhCDR3 having an amino acid sequence of SEQ ID NO:329; and (ii) a VL comprising a vlCDR1 having an amino acid sequence of SEQ ID NO:331, a vlCDR2 having an amino acid sequence of SEQ ID NO: 332, and a vlCDR3 having an amino acid sequence of SEQ ID NO:333; or
(i) a VH comprising a vhCDR1 having an amino acid sequence of SEQ ID NO:335; a vhCDR2 having an amino acid sequence of SEQ ID NO:336; and a vhCDR3 having an amino acid sequence of SEQ ID NO:337; and (ii) a VL comprising a vlCDR1 having an amino acid sequence of SEQ ID NO:339, a vlCDR2 having an amino acid sequence of SEQ ID NO: 340, and a vlCDR3 having an amino acid sequence of SEQ ID NO:341.

In some embodiments, the VH and VL of the PSMA binding domain(s) are selected from the following:
(i) a VH1 having an amino acid sequence that is at least about 95% identical to SEQ ID NO:206; and (ii) a VL1 having an amino acid sequence that is at least about 95% identical to SEQ ID NO:210; or
(i) a VH1 having an amino acid sequence that is at least about 95% identical to SEQ ID NO:214; and (ii) a VL1 having an amino acid sequence that is at least about 95% identical to SEQ ID NO:218; or
(i) a VH1 having an amino acid sequence that is at least about 95% identical to SEQ ID NO:222; and (ii) a VL1 having an amino acid sequence that is at least about 95% identical to SEQ ID NO:226; or
(i) a VH1 having an amino acid sequence that is at least about 95% identical to SEQ ID NO:230; and (ii) a VL1 having an amino acid sequence that is at least about 95% identical to SEQ ID NO:234; or
(i) a VH1 having an amino acid sequence that is at least about 95% identical to SEQ ID NO:238; and (ii) a VL1 having an amino acid sequence that is at least about 95% identical to SEQ ID NO:242; or
(i) a VH1 having an amino acid sequence that is at least about 95% identical to SEQ ID NO:246; and (ii) a VL1 having an amino acid sequence that is at least about 95% identical to SEQ ID NO:250; or
(i) a VH1 having an amino acid sequence that is at least about 95% identical to SEQ ID NO:254; and (ii) a VL1 having an amino acid sequence that is at least about 95% identical to SEQ ID NO:258; or
(i) a VH1 having an amino acid sequence that is at least about 95% identical to SEQ ID NO:262; and (ii) a VL1 having an amino acid sequence that is at least about 95% identical to SEQ ID NO:266; or
(i) a VH1 having an amino acid sequence that is at least about 95% identical to SEQ ID NO:270; and (ii) a VL1 having an amino acid sequence that is at least about 95% identical to SEQ ID NO:274; or
(i) a VH1 having an amino acid sequence that is at least about 95% identical to SEQ ID NO:278; and (ii) a VL1 having an amino acid sequence that is at least about 95% identical to SEQ ID NO:282; or
(i) a VH1 having an amino acid sequence that is at least about 95% identical to SEQ ID NO:286; and (ii) a VL1 having an amino acid sequence that is at least about 95% identical to SEQ ID NO:290; or
(i) a VH1 having an amino acid sequence that is at least about 95% identical to SEQ ID NO:294; and (ii) a VL1 having an amino acid sequence that is at least about 95% identical to SEQ ID NO:298; or
(i) a VH1 having an amino acid sequence that is at least about 95% identical to SEQ ID NO:302; and (ii) a VL1 having an amino acid sequence that is at least about 95% identical to SEQ ID NO:306; or
(i) a VH1 having an amino acid sequence that is at least about 95% identical to SEQ ID NO:310; and (ii) a VL1 having an amino acid sequence that is at least about 95% identical to SEQ ID NO:314; or
(i) a VH1 having an amino acid sequence that is at least about 95% identical to SEQ ID NO:318; and (ii) a VL1 having an amino acid sequence that is at least about 95% identical to SEQ ID NO:322; or
(i) a VH1 having an amino acid sequence that is at least about 95% identical to SEQ ID NO:326; and (ii) a VL1 having an amino acid sequence that is at least about 95% identical to SEQ ID NO:330; or
(i) a VH1 having an amino acid sequence that is at least about 95% identical to SEQ ID NO:334; and (ii) a VL1 having an amino acid sequence that is at least about 95% identical to SEQ ID NO:338; and

In some embodiments, the VH and VL of the PSMA binding domain(s) are selected from the following:
(i) a VH having an amino acid sequence of SEQ ID NO:206; and (ii) a VL having an amino acid sequence of SEQ ID NO:210; or
(i) a VH having an amino acid sequence of SEQ ID NO:214; and (ii) a VL having an amino acid sequence of SEQ ID NO:218; or
(i) a VH having an amino acid sequence of SEQ ID NO:222; and (ii) a VL having an amino acid sequence of SEQ ID NO:226; or
(i) a VH having an amino acid sequence of SEQ ID NO:230; and (ii) a VL having an amino acid sequence of SEQ ID NO:234; or
(i) a VH having an amino acid sequence of SEQ ID NO:238; and (ii) a VL having an amino acid sequence of SEQ ID NO:242; or
(i) a VH having an amino acid sequence of SEQ ID NO:246; and (ii) a VL having an amino acid sequence of SEQ ID NO:250; or
(i) a VH having an amino acid sequence of SEQ ID NO:254; and (ii) a VL having an amino acid sequence of SEQ ID NO:258; or
(i) a VH having an amino acid sequence of SEQ ID NO:262; and (ii) a VL having an amino acid sequence of SEQ ID NO:266; or
(i) a VH having an amino acid sequence of SEQ ID NO:270; and (ii) a VL having an amino acid sequence of SEQ ID NO:274; or
(i) a VH having an amino acid sequence of SEQ ID NO:278; and (ii) a VL having an amino acid sequence of SEQ ID NO:282; or
(i) a VH having an amino acid sequence of SEQ ID NO:286; and (ii) a VL having an amino acid sequence of SEQ ID NO:290; or
(i) a VH having an amino acid sequence of SEQ ID NO:294; and (ii) a VL having an amino acid sequence of SEQ ID NO:298; or
(i) a VH having an amino acid sequence of SEQ ID NO:302; and (ii) a VL having an amino acid sequence of SEQ ID NO:306; or
(i) a VH having an amino acid sequence of SEQ ID NO:310; and (ii) a VL having an amino acid sequence of SEQ ID NO:314; or
(i) a VH having an amino acid sequence of SEQ ID NO:318; and (ii) a VL having an amino acid sequence of SEQ ID NO:322; or
(i) a VH having an amino acid sequence of SEQ ID NO:326; and (ii) a VL having an amino acid sequence of SEQ ID NO:330; or
(i) a VH having an amino acid sequence of SEQ ID NO:334; and (ii) a VL having an amino acid sequence of SEQ ID NO:338.

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10−20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294: 151-162; Baca et al., 1997, J. Biol. Chem. 272(16): 10678-10684; Rosok et al., 1996, J. Biol. Chem. 271(37): 22611-22618; Rader et al., 1998, Proc. Natl. Acad. Sci. USA 95: 8910−8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Heterodimeric Antibodies

In exemplary embodiments, the anti-CD28 x anti-PSMA antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

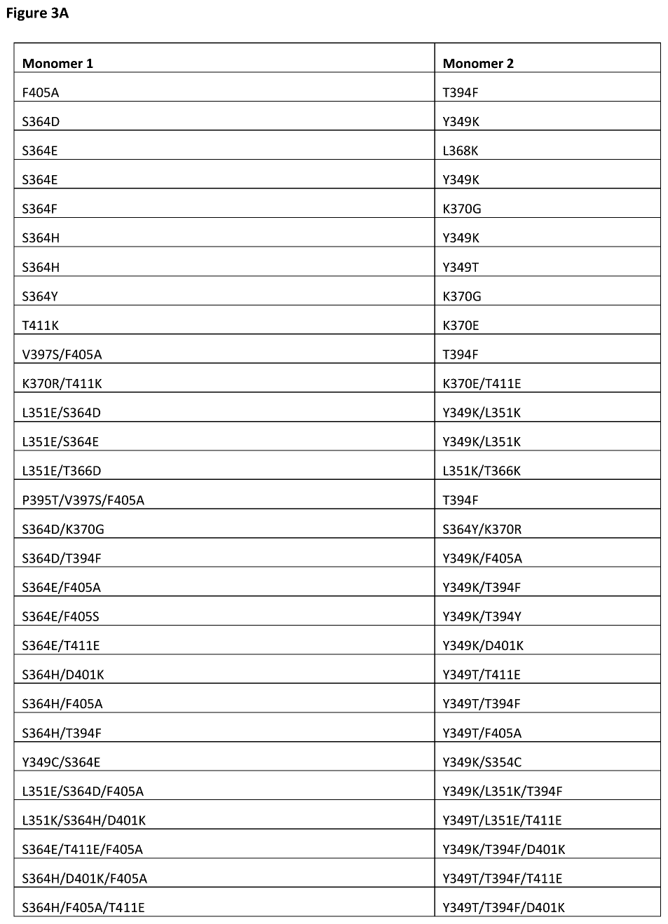

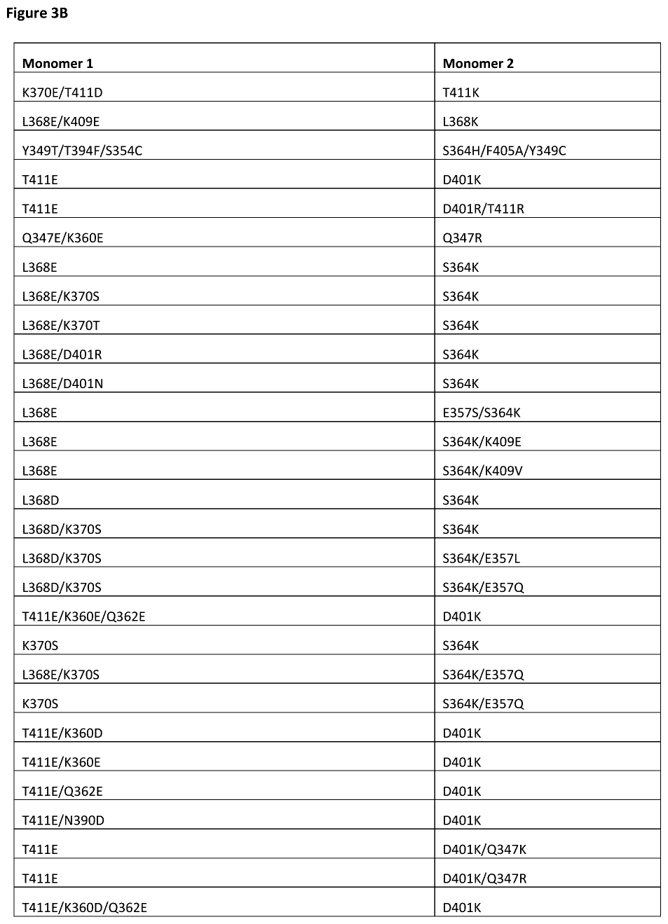

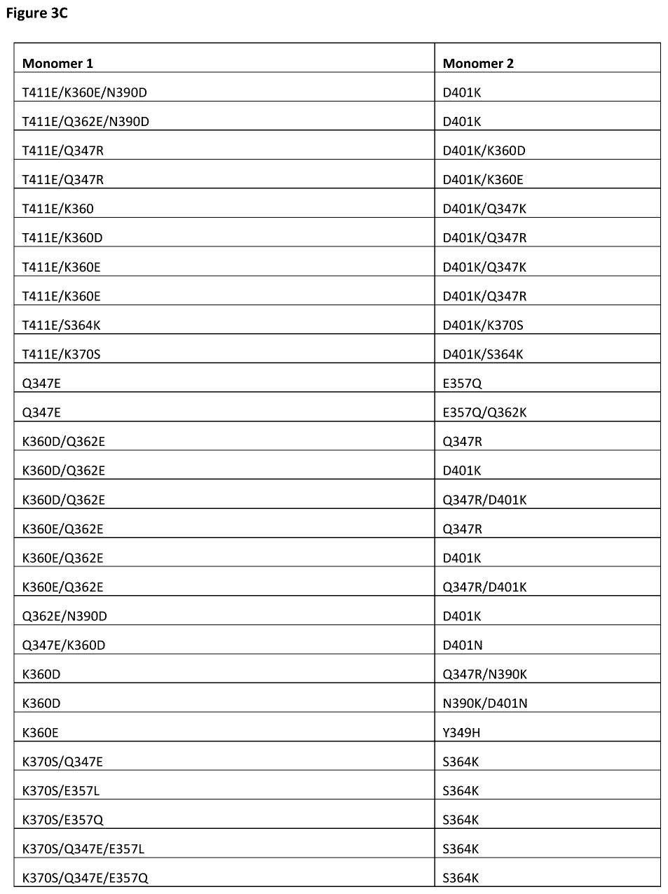

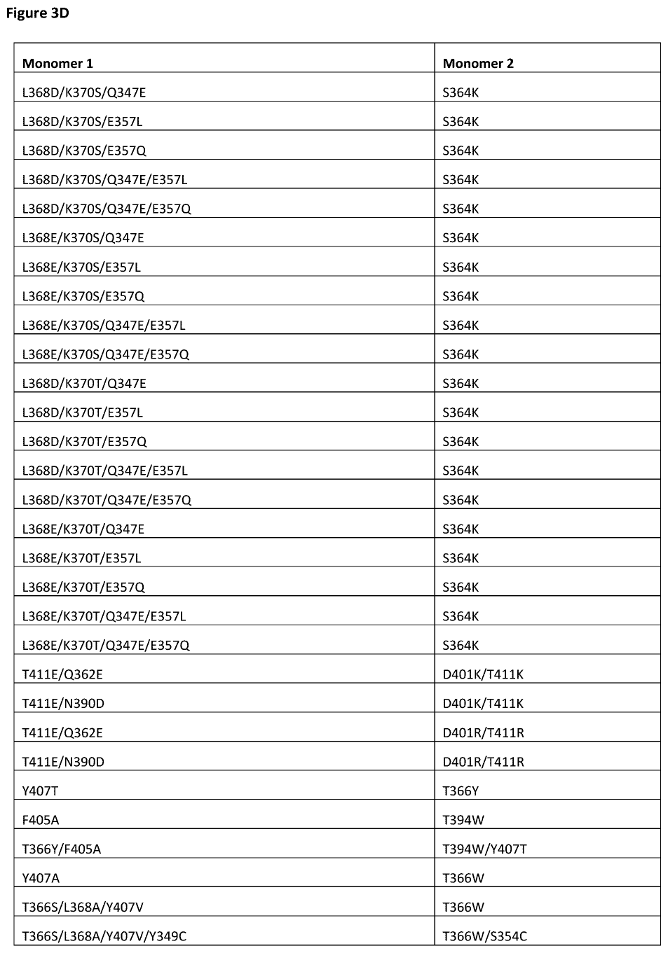

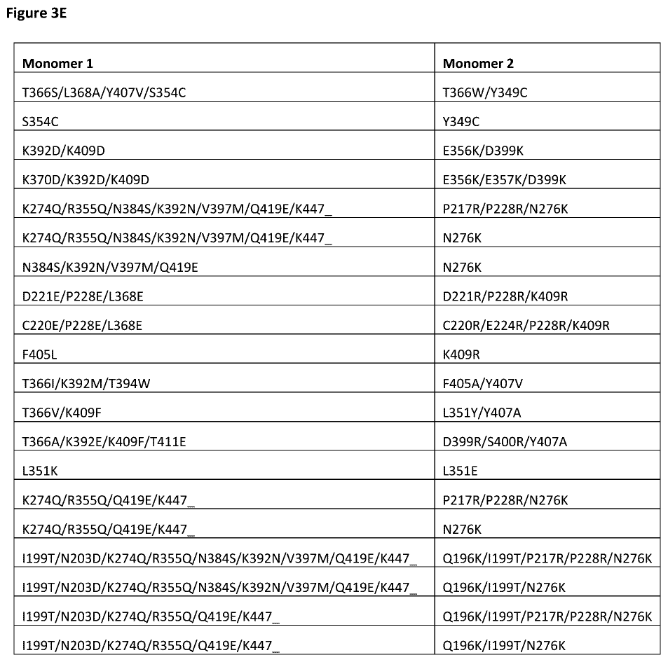

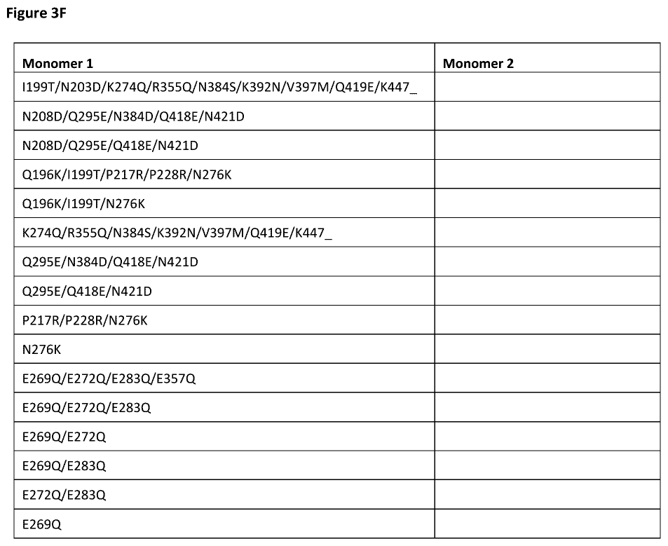

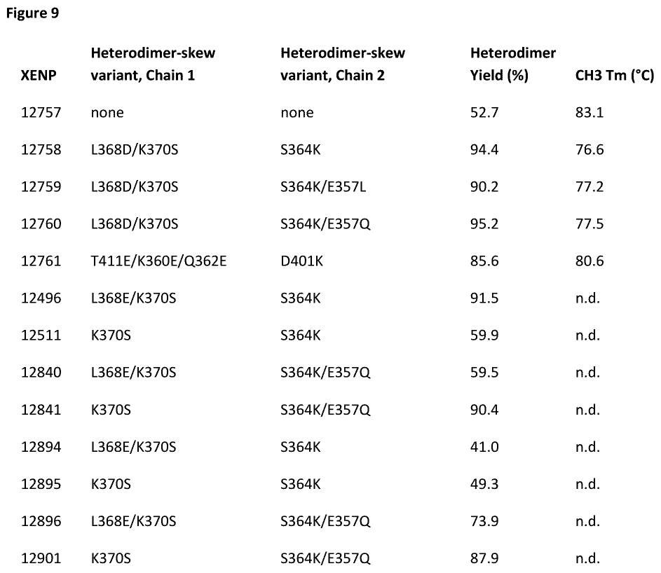

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 3A-3F and 9.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A—monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25): 19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIGS. 3A-3F. Such “skew” variants include, but are not limited to: S364K/E357Q: L368D/K370S; L368D/K370S: S364K; L368E/K370S: S364K; T411T/E360E/Q362E: D401K; L368D/K370S: S364K/E357L; K370S: S364K/E357Q (EU numbering).

In exemplary embodiments, the heterodimeric antibody includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C) “skew” variant amino acid substitution set (EU numbering). In an exemplary embodiment, the heterodimeric antibody includes a “S364K/E357Q: L368D/K370S” amino acid substitution set. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

### 2. Purification Variants

In some embodiments, the heterodimeric antibody includes purification variants that advantageously allow for the separation of heterodimeric proteins (e.g., anti-CD28 x anti-PSMA bispecific antibody) from homodimeric proteins.

There are several basic mechanisms that can lead to case of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, and the “2+1 Fab2-scFv-Fc” format, allows separation on the basis of size. As described above, it is also possible to “skew” the formation of heterodimers over homodimers using skew variants. Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab2-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

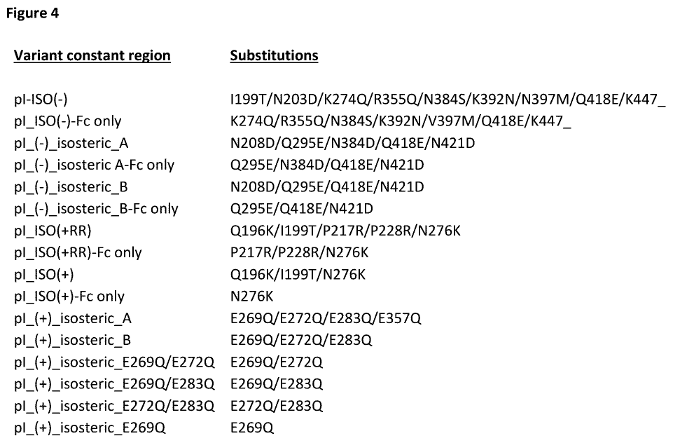

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine.). A number of these variants are shown in the FIGS. 3A-3F and 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab2-scFv-Fc, 1+1 CLC and 2+1 CLC formats, the starting pI of the scFv (1+1 Fab-scFv-Fc, 2+1 Fab2-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. Preferred combinations of pI variants are shown in FIGS. 3A-3F and 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)+(SEQ ID NO:443). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 8 and the other monomer has a charged linker (either in the form of a charged scFv linker because that monomer comprises an scFv or a charged domain linker, as the format dictates, which can be selected from those depicted in FIG. 6A-6B).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-CD28 x anti-PSMA antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). Sec, e.g., US2010331527, which is incorporated by reference in its entirety, and specifically for pertinent disclosures relating to Fc domain modifications to reduce Protein A binding.

### 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is more further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. Nos. 8,188,321 (particularly FIG. 41) and 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 2591, 428L/434S, 2591/308F, 4361/428L, 4361 or V/434S, 436V/428L, 2591/308F/428L, and M252Y/S254T/T256E. Such modification may be included in one or both Fc domains of the subject antibody.

### 2. Ablation Variants

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 5, and each can be independently and optionally included or excluded, with preferred aspects utilizing ablation variants selected from the group consisting of: L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or purification variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

Exemplary combination of variants that are included in some embodiments of the heterodimeric 1+1 Fab-scFv-Fc, and 2+1 Fab2-scFv-Fc format antibodies are included in FIG. 8. In some embodiments, the heterodimeric antibody includes a combination of variants as depicted in FIG. 8. In certain embodiments, the antibody is a heterodimeric 1+1 Fab-scFv-Fc, or 2+1 Fab2-scFv-Fc format antibody that includes the “platform X” combination of variants depicted in FIG. 8. In certain embodiments, the antibody is a heterodimeric 1+1 Fab-scFv-Fc, or 2+1 Fab2-scFv-Fc format antibody that includes the “platform J” combination of variants depicted in FIG. 8.

### G. Useful Antibody Formats

As will be appreciated by those in the art and discussed more fully below, the heterodimeric bispecific antibodies provided herein can take on several different configurations as generally depicted in FIG. 24A-24M.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain. As is outlined herein, when CD28 is one of the target antigens, it is preferable that the CD28 is bound only monovalently.

The present invention utilizes CD28 binding domains in combination with PSMA binding domains. As will be appreciated by those in the art, any collection of anti-CD28 CDRs, anti-CD28 variable light and variable heavy domains, Fabs and scFvs as depicted in any of the Figures (see particularly FIGS. 15-18C, and 21A-21H) can be used. Similarly, any of the PSMA antigen binding domains can be used, whether CDRs, variable light and variable heavy domains, Fabs and scFvs as depicted in any of the Figures (e.g., FIGS. 25A-25E and 26A-26B) can be used, optionally and independently combined in any combination.

1. 1+1 Fab-scFv-Fc format

One heterodimeric antibody format that finds particular use in subject anti-CD28 x anti-PSMA antibodies provided herein is the “1+1 Fab-scFv-Fc” or “bottle opener” format as shown in FIG. 24A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv”, as defined below) and a second Fc domain. The scFv includes a second variable heavy domain (VH2) and a second variable light domain (VL2), wherein the VH2 is attached to the VL2 using an scFv linker that can be charged (see, e.g., FIGS. 6A-6B). The scFv is attached to the heavy chain using a domain linker (see, e.g., FIG. 7). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a CD28 binding domain and the other binding domain is a PSMA binding domain. In some embodiments where the 1+1 Fab-scFv-Fc, it is the scFv that binds to the CD28, and the Fab that binds PSMA. Exemplary anti-CD28 x anti-PSMA bispecific antibodies in the 1+1 Fab-scFv-Fc format are depicted in FIG. 29A-29RR and 30A-30L.

In some embodiments, the first and second Fc domains of the 1+1 Fab-scFv-Fc format antibody are variant Fc domains that include heterodimerization skew variants (e.g., a set of amino acid substitutions as shown in FIGS. 3A-3F and 9). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody include “Platform X” amino acid modifications as depicted in FIG. 8. In such embodiments, the CH1-hinge-CH2-CH3 of the first monomer comprises amino acid variants L368D/K370S/N208D/Q295E/N384D/Q418E/N421D/E233P/L234V/L235A/G236del/S267 K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIGS. 6A-6B). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody with “Platform X” variants, the first Fc domain includes heterodimerization skew variants L368D/K370S and the second Fc domain includes heterodimerization skew variants S364K/E357Q; each of the first and second Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)4 charged scFv linker (SEQ ID NO:443). In some embodiments, the 1+1 Fab-scFv-Fc format antibody provided herein includes FcRn variants M428L/N434S, wherein numbering is according to EU numbering. In some embodiments, the scFv of the 1 +1 Fab-scFv-Fc format antibody provided herein includes a charged scFv linker (including those shown in FIGS. 6A-6B).

In exemplary embodiments, the 1+1 Fab-scFv-Fc format antibody include “Platform J” amino acid modifications as depicted in FIG. 8. In such embodiments, the first Fc domain comprises amino acid substitutions L234A/L235A/D265S/T366S/L368A/Y407V/H435R/Y436F, and the first Fc domain comprises amino acid substitutions L234A/L235A/D265S/LT366W, wherein numbering is according to EU numbering.

In exemplary embodiments 1+1 Fab-scFv-Fc format antibody with Platform J variants, the first Fc domain includes heterodimerization skew variants T366S/L368A/Y407V and the second Fc domain includes heterodimerization skew variants T366W; each of the first and second variant Fc domains include ablation variants L234A/L235A/D265S; and the second Fc domain includes purification variants H435R/Y436F, wherein numbering is according to EU numbering. In some embodiments, the scFv of the 1+1 Fab-scFv-Fc format antibody provided herein includes a (GKPGS)4 charged scFv linker (SEQ ID NO:443). In some embodiments, the 1+1 Fab-scFv-Fc format antibody provided herein includes FcRn variants M252Y/S254T/T256E, wherein numbering is according to EU numbering. In some embodiments, the scFv of the 1+1 Fab-scFv-Fc format antibody provided herein includes a charged scFv linker (including those shown in FIGS. 6A-6B).

In exemplary embodiments, the 1+1 Fab-scFv-Fc format antibody with Platform J variants further includes a “stapled” scFv (e.g., a “stapled” anti-CD28 scFv). Stapled” scFvs that exhibit improved stability and/or reduced aggregation are further described in detail herein. Exemplary staple linkers that are useful for inclusion in such “stapled” scFvs are provided in FIGS. 6A-6B. In exemplary embodiments, the stapled linker is GGGSGGSGGCPPCGGSGG (SEQ ID NO:457).

In some embodiments, one of the first binding domain or the second binding domain binds CD28 and the other binding domain binds a tumor associated antigen (TAA) (see FIG. 34A). Any suitable CD28 binding domain can be included in subject 1+1 Fab-scFv-Fc format antibody, including any of the CD28 binding domains provided herein. In some embodiments, the CD28 binding domain is one of the following CD28 binding domains or a variant thereof: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_HIL1, 9G2[CD28]_H0L0, 9G2[CD28]_HIL1, 2F10A3.140[CD28]_HIL1, and TN228[CD28]_H4L2 (FIGS. 15-18C, 21A-21H, and 69A-69C).

In some embodiments of the 1+1 Fab-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:13; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:31; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:35; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and (ii) a VL comprising a vlCDR1, a vlCDR2, and a vCDR3 having an amino acid sequence of a vlCDR1, a vlCDR2, and a vlCDR3, respectively, of a VL having an amino acid sequence of SEQ ID NO:43; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:47; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:51; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:55; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:59; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:67; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:90; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:94; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:98; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 102; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 106; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:110; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:114; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:118; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 122; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:126; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:130; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 134; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:138; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 142; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 146; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 150; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:158; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:166; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 170; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 174; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 178; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 182; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:186; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 190; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 194; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 198; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:202; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:380; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:384; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:396; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:400; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:404; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:408; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:412; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:416; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:420; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:424.

In some embodiments of the 1+1 Fab-scFv-Fc format, the anti-CD28 ABD has a VH and VL domain selected from the following:
(i) a VH comprising a vhCDR1 having an amino acid sequence of SEQ ID NO:10; a vhCDR2 having an amino acid sequence of SEQ ID NO:11; and a vhCDR3 having an amino acid sequence of SEQ ID NO:12; and (ii) a VL comprising a vlCDR1 having an amino acid sequence of SEQ ID NO:14, a vlCDR2 having an amino acid sequence of SEQ ID NO: 15, and a vlCDR3 having an amino acid sequence of SEQ ID NO: 16; or
(i) a VH comprising a vhCDR1 having an amino acid sequence of SEQ ID NO:32; a vhCDR2 having an amino acid sequence of SEQ ID NO:33; and a vhCDR3 having an amino acid sequence of SEQ ID NO:34; and (ii) a VL comprising a vlCDR1 having an amino acid sequence of SEQ ID NO:36, a vlCDR2 having an amino acid sequence of SEQ ID NO: 37, and a vlCDR3 having an amino acid sequence of SEQ ID NO:38; or
i) a VH comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; or
i) a VH comprising a vhCDR1 having an amino acid sequence of SEQ ID NO:48; a vhCDR2 having an amino acid sequence of SEQ ID NO:49; and a vhCDR3 having an amino acid sequence of SEQ ID NO:50; and (ii) a VL comprising a vlCDR1 having an amino acid sequence of SEQ ID NO:52, a vlCDR2 having an amino acid sequence of SEQ ID NO: 53, and a vlCDR3 having an amino acid sequence of SEQ ID NO:54; or
i) a VH comprising a vhCDR1 having an amino acid sequence of SEQ ID NO:56; a vhCDR2 having an amino acid sequence of SEQ ID NO:57; and a vhCDR3 having an amino acid sequence of SEQ ID NO:58; and (ii) a VL comprising a vlCDR1 having an amino acid sequence of SEQ ID NO:60, a vlCDR2 having an amino acid sequence of SEQ ID NO:61, and a vlCDR3 having an amino acid sequence of SEQ ID NO:62; or
i) a VH comprising a vhCDR1 having an amino acid sequence of SEQ ID NO:64; a vhCDR2 having an amino acid sequence of SEQ ID NO:65; and a vhCDR3 having an amino acid sequence of SEQ ID NO:66; and (ii) a VL comprising a vlCDR1 having an amino acid sequence of SEQ ID NO:68, a vlCDR2 having an amino acid sequence of SEQ ID NO:69, and a vlCDR3 having an amino acid sequence of SEQ ID NO:70; or
i) a VH comprising a vhCDR1 having an amino acid sequence of SEQ ID NO:87; a vhCDR2 having an amino acid sequence of SEQ ID NO:88; and a vhCDR3 having an amino acid sequence of SEQ ID NO:89; and (ii) a VL comprising a vlCDR1 having an amino acid sequence of SEQ ID NO:91, a vlCDR2 having an amino acid sequence of SEQ ID NO:92, and a vlCDR3 having an amino acid sequence of SEQ ID NO:93; or
i) a VH comprising a vhCDR1 having an amino acid sequence of SEQ ID NO:95; a vhCDR2 having an amino acid sequence of SEQ ID NO:96; and a vhCDR3 having an amino acid sequence of SEQ ID NO:97; and (ii) a VL comprising a vlCDR1 having an amino acid sequence of SEQ ID NO:99, a vlCDR2 having an amino acid sequence of SEQ ID NO:100, and a vlCDR3 having an amino acid sequence of SEQ ID NO:101; or
i) a VH comprising a vhCDR1 having an amino acid sequence of SEQ ID NO:103; a vhCDR2 having an amino acid sequence of SEQ ID NO: 104; and a vhCDR3 having an amino acid sequence of SEQ ID NO: 105; and (ii) a VL comprising a vlCDR1 having an amino acid sequence of SEQ ID NO: 107, a vlCDR2 having an amino acid sequence of SEQ ID NO:108, and a vlCDR3 having an amino acid sequence of SEQ ID NO:109; or
i) a VH comprising a vhCDR1 having an amino acid sequence of SEQ ID NO:111; a vhCDR2 having an amino acid sequence of SEQ ID NO:112; and a vhCDR3 having an amino acid sequence of SEQ ID NO:113; and (ii) a VL comprising a vlCDR1 having an amino acid sequence of SEQ ID NO:115, a vlCDR2 having an amino acid sequence of SEQ ID NO:116, and a vlCDR3 having an amino acid sequence of SEQ ID NO:117; or
i) a VH comprising a vhCDR1 having an amino acid sequence of SEQ ID NO:119; a vhCDR2 having an amino acid sequence of SEQ ID NO:120; and a vhCDR3 having an amino acid sequence of SEQ ID NO:121; and (ii) a VL comprising a vlCDR1 having an amino acid sequence of SEQ ID NO: 123, a vlCDR2 having an amino acid sequence of SEQ ID NO: 124, and a vlCDR3 having an amino acid sequence of SEQ ID NO:125; or
i) a VH comprising a vhCDR1 having an amino acid sequence of SEQ ID NO:127; a vhCDR2 having an amino acid sequence of SEQ ID NO:128; and a vhCDR3 having an amino acid sequence of SEQ ID NO: 129; and (ii) a VL comprising a vlCDR1 having an amino acid sequence of SEQ ID NO:131, a vlCDR2 having an amino acid sequence of SEQ ID NO: 132, and a vlCDR3 having an amino acid sequence of SEQ ID NO:133; or
i) a VH comprising a vhCDR1 having an amino acid sequence of SEQ ID NO:135; a vhCDR2 having an amino acid sequence of SEQ ID NO:136; and a vhCDR3 having an amino acid sequence of SEQ ID NO:137; and (ii) a VL comprising a vlCDR1 having an amino acid sequence of SEQ ID NO: 139, a vlCDR2 having an amino acid sequence of SEQ ID NO: 140, and a vlCDR3 having an amino acid sequence of SEQ ID NO: 141; or
i) a VH comprising a vhCDR1 having an amino acid sequence of SEQ ID NO:143; a vhCDR2 having an amino acid sequence of SEQ ID NO:144; and a vhCDR3 having an amino acid sequence of SEQ ID NO:115; and (ii) a VL comprising a vlCDR1 having an amino acid sequence of SEQ ID NO: 147, a vlCDR2 having an amino acid sequence of SEQ ID NO: 149, and a vlCDR3 having an amino acid sequence of SEQ ID NO: 149; or
i) a VH comprising a vhCDR1 having an amino acid sequence of SEQ ID NO:151; a vhCDR2 having an amino acid sequence of SEQ ID NO:152; and a vhCDR3 having an amino acid sequence of SEQ ID NO: 153; and (ii) a VL comprising a vlCDR1 having an amino acid sequence of SEQ ID NO:155, a vlCDR2 having an amino acid sequence of SEQ ID NO:156, and a vlCDR3 having an amino acid sequence of SEQ ID NO:157; or
i) a VH comprising a vhCDR1 having an amino acid sequence of SEQ ID NO:159; a vhCDR2 having an amino acid sequence of SEQ ID NO: 160; and a vhCDR3 having an amino acid sequence of SEQ ID NO: 161; and (ii) a VL comprising a vlCDR1 having an amino acid sequence of SEQ ID NO:163, a vlCDR2 having an amino acid sequence of SEQ ID NO:164, and a vlCDR3 having an amino acid sequence of SEQ ID NO:165; or
i) a VH comprising a vhCDR1 having an amino acid sequence of SEQ ID NO:167; a vhCDR2 having an amino acid sequence of SEQ ID NO:168; and a vhCDR3 having an amino acid sequence of SEQ ID NO:169; and (ii) a VL comprising a vlCDR1 having an amino acid sequence of SEQ ID NO:171, a vlCDR2 having an amino acid sequence of SEQ ID NO:172, and a vlCDR3 having an amino acid sequence of SEQ ID NO:173; or
i) a VH comprising a vhCDR1 having an amino acid sequence of SEQ ID NO:175; a vhCDR2 having an amino acid sequence of SEQ ID NO:176; and a vhCDR3 having an amino acid sequence of SEQ ID NO: 177; and (ii) a VL comprising a vlCDR1 having an amino acid sequence of SEQ ID NO:179, a vlCDR2 having an amino acid sequence of SEQ ID NO:180, and a vlCDR3 having an amino acid sequence of SEQ ID NO:181; or
i) a VH comprising a vhCDR1 having an amino acid sequence of SEQ ID NO:183; a vhCDR2 having an amino acid sequence of SEQ ID NO:184; and a vhCDR3 having an amino acid sequence of SEQ ID NO:185; and (ii) a VL comprising a vlCDR1 having an amino acid sequence of SEQ ID NO: 187, a vlCDR2 having an amino acid sequence of SEQ ID NO:188, and a vlCDR3 having an amino acid sequence of SEQ ID NO:189; or
i) a VH comprising a vhCDR1 having an amino acid sequence of SEQ ID NO:191; a vhCDR2 having an amino acid sequence of SEQ ID NO:192; and a vhCDR3 having an amino acid sequence of SEQ ID NO: 193; and (ii) a VL comprising a vlCDR1 having an amino acid sequence of SEQ ID NO: 195, a vlCDR2 having an amino acid sequence of SEQ ID NO: 196, and a vlCDR3 having an amino acid sequence of SEQ ID NO:197; or
i) a VH comprising a vhCDR1 having an amino acid sequence of SEQ ID NO:199; a vhCDR2 having an amino acid sequence of SEQ ID NO:200; and a vhCDR3 having an amino acid sequence of SEQ ID NO:201; and (ii) a VL comprising a vlCDR1 having an amino acid sequence of SEQ ID NO:203, a vlCDR2 having an amino acid sequence of SEQ ID NO:204, and a vlCDR3 having an amino acid sequence of SEQ ID NO:205; or
i) a VH comprising a vhCDR1 having an amino acid sequence of SEQ ID NO:381; a vhCDR2 having an amino acid sequence of SEQ ID NO:382; and a vhCDR3 having an amino acid sequence of SEQ ID NO:383; and (ii) a VL comprising a vlCDR1 having an amino acid sequence of SEQ ID NO:385, a vlCDR2 having an amino acid sequence of SEQ ID NO:386, and a vlCDR3 having an amino acid sequence of SEQ ID NO:387; or
i) a VH comprising a vhCDR1 having an amino acid sequence of SEQ ID NO:389; a vhCDR2 having an amino acid sequence of SEQ ID NO:390; and a vhCDR3 having an amino acid sequence of SEQ ID NO:391; and (ii) a VL comprising a vlCDR1 having an amino acid sequence of SEQ ID NO:393, a vlCDR2 having an amino acid sequence of SEQ ID NO:394, and a vlCDR3 having an amino acid sequence of SEQ ID NO:395; or
i) a VH comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403; or
i) a VH comprising a vhCDR1 having an amino acid sequence of SEQ ID NO:405; a vhCDR2 having an amino acid sequence of SEQ ID NO:406; and a vhCDR3 having an amino acid sequence of SEQ ID NO:407; and (ii) a VL comprising a vlCDR1 having an amino acid sequence of SEQ ID NO:409, a vlCDR2 having an amino acid sequence of SEQ ID NO:410, and a vlCDR3 having an amino acid sequence of SEQ ID NO:411; or
i) a VH comprising a vhCDR1 having an amino acid sequence of SEQ ID NO:413; a vhCDR2 having an amino acid sequence of SEQ ID NO:414; and a vhCDR3 having an amino acid sequence of SEQ ID NO:415; and (ii) a VL comprising a vlCDR1 having an amino acid sequence of SEQ ID NO:417, a vlCDR2 having an amino acid sequence of SEQ ID NO:418, and a vlCDR3 having an amino acid sequence of SEQ ID NO:419; or
i) a VH comprising a vhCDR1 having an amino acid sequence of SEQ ID NO:421; a vhCDR2 having an amino acid sequence of SEQ ID NO:422; and a vhCDR3 having an amino acid sequence of SEQ ID NO:423; and (ii) a VL comprising a vlCDR1 having an amino acid sequence of SEQ ID NO:425, a vlCDR2 having an amino acid sequence of SEQ ID NO:426, and a vlCDR3 having an amino acid sequence of SEQ ID NO:427.

In some embodiments of the 1+1 Fab-scFv-Fc format, the anti-CD28 ABD has a VH and VL domain selected from the following:
(i) a VH having an amino acid sequence of SEQ ID NO:9; and (ii) a VL having an amino acid sequence of SEQ ID NO:13; or
(i) a VH having an amino acid sequence of SEQ ID NO:31; and (ii) a VL having an amino acid sequence of SEQ ID NO:35; or
(i) a VH having an amino acid sequence of SEQ ID NO:39; and (ii) a VL having an amino acid sequence of SEQ ID NO:43; or
(i) a VH having an amino acid sequence of SEQ ID NO:47; and (ii) a VL having an amino acid sequence of SEQ ID NO:51; or
(i) a VH having an amino acid sequence of SEQ ID NO:55; and (ii) a VL having an amino acid sequence of SEQ ID NO:59; or
(i) a VH having an amino acid sequence of SEQ ID NO:63; and (ii) a VL having an amino acid sequence of SEQ ID NO:67; or
(i) a VH having an amino acid sequence of SEQ ID NO:86; and (ii) a VL having an amino acid sequence of SEQ ID NO:90; or
(i) a VH having an amino acid sequence of SEQ ID NO:94; and (ii) a VL having an amino acid sequence of SEQ ID NO:98; or
(i) a VH having an amino acid sequence of SEQ ID NO:102; and (ii) a VL having an amino acid sequence of SEQ ID NO: 106; or
(i) a VH having an amino acid sequence of SEQ ID NO:110; and (ii) a VL having an amino acid sequence of SEQ ID NO:114; or
(i) a VH having an amino acid sequence of SEQ ID NO:118; and (ii) a VL having an amino acid sequence of SEQ ID NO:122; or
(i) a VH having an amino acid sequence of SEQ ID NO:126; and (ii) a VL having an amino acid sequence of SEQ ID NO:130; or
(i) a VH having an amino acid sequence of SEQ ID NO:134; and (ii) a VL having an amino acid sequence of SEQ ID NO:138; or
(i) a VH having an amino acid sequence of SEQ ID NO:142; and (ii) a VL having an amino acid sequence of SEQ ID NO: 146; or
(i) a VH having an amino acid sequence of SEQ ID NO:150; and (ii) a VL having an amino acid sequence of SEQ ID NO:154; or
(i) a VH having an amino acid sequence of SEQ ID NO:158; and (ii) a VL having an amino acid sequence of SEQ ID NO:162; or
(i) a VH having an amino acid sequence of SEQ ID NO:166; and (ii) a VL having an amino acid sequence of SEQ ID NO:170; or
(i) a VH having an amino acid sequence of SEQ ID NO:174; and (ii) a VL having an amino acid sequence of SEQ ID NO: 178; or
(i) a VH having an amino acid sequence of SEQ ID NO:182; and (ii) a VL having an amino acid sequence of SEQ ID NO: 186; or
(i) a VH having an amino acid sequence of SEQ ID NO: 190; and (ii) a VL having an amino acid sequence of SEQ ID NO: 194; or
(i) a VH having an amino acid sequence of SEQ ID NO: 198; and (ii) a VL having an amino acid sequence of SEQ ID NO:202; or
(i) a VH having an amino acid sequence of SEQ ID NO:380; and (ii) a VL having an amino acid sequence of SEQ ID NO:384; or
(i) a VH having an amino acid sequence of SEQ ID NO:388; and (ii) a VL having an amino acid sequence of SEQ ID NO:392; or
(i) a VH having an amino acid sequence of SEQ ID NO:396; and (ii) a VL having an amino acid sequence of SEQ ID NO:400; or
(i) a VH having an amino acid sequence of SEQ ID NO:404; and (ii) a VL having an amino acid sequence of SEQ ID NO:408; or
(i) a VH having an amino acid sequence of SEQ ID NO:412; and (ii) a VL having an amino acid sequence of SEQ ID NO:416; or
(i) a VH having an amino acid sequence of SEQ ID NO:420; and (ii) a VL having an amino acid sequence of SEQ ID NO:424.

In some embodiments, one of the first binding domain or the second binding domain of the 1+1 Fab-scFv-Fc format antibody binds PSMA. In some embodiments, the PSMA binding domain is one of the following PSMA binding domains or a variant thereof: A10[PSMA], A10v2[PSMA], DO1[PSMA], DO1v2[PSMA], E07[PSMA], F02[PSMA], A11[PSMA], F07[PSMA], F07v2[PSMA], G02[PSMA], F01[PSMA], F01v2[PSMA], 011A11[PSMA], PSMB896[PSMA], PSMA-H[PSMA], and D7[PSMA] (FIGS. 25A-25E and 26A-26B).

In some embodiments of the 1+1 Fab-scFv-Fc format, the anti-PSMA ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:206; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:210; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:214; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:218; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:222; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:226; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:230; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:234; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:238; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:242; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:246; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:250; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:254; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:258; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:262; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:266; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:270; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:274; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:278; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:282; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:286; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:290; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:294; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:298; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:302; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:306; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:310; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:314; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:318; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:322; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:326; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:330; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:334; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:338.

In some embodiments of the 1+1 Fab-scFv-Fc format, the anti-PSMA ABD has a VH and VL domain selected from the following:
(i) a VH comprising a vhCDR1 having an amino acid sequence of SEQ ID NO:207; a vhCDR2 having an amino acid sequence of SEQ ID NO:208; and a vhCDR3 having an amino acid sequence of SEQ ID NO:209; and (ii) a VL comprising a vlCDR1 having an amino acid sequence of SEQ ID NO:211, a vlCDR2 having an amino acid sequence of SEQ ID NO: 212, and a vlCDR3 having an amino acid sequence of SEQ ID NO:213; or
(i) a VH comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221;
(i) a VH comprising a vhCDR1 having an amino acid sequence of SEQ ID NO:223; a vhCDR2 having an amino acid sequence of SEQ ID NO:224; and a vhCDR3 having an amino acid sequence of SEQ ID NO:225; and (ii) a VL comprising a vlCDR1 having an amino acid sequence of SEQ ID NO:227, a vlCDR2 having an amino acid sequence of SEQ ID NO: 228, and a vlCDR3 having an amino acid sequence of SEQ ID NO:229; or
(i) a VH comprising a vhCDR1 having an amino acid sequence of SEQ ID NO:231; a vhCDR2 having an amino acid sequence of SEQ ID NO:232; and a vhCDR3 having an amino acid sequence of SEQ ID NO:233; and (ii) a VL comprising a vlCDR1 having an amino acid sequence of SEQ ID NO:235, a vlCDR2 having an amino acid sequence of SEQ ID NO: 236, and a vlCDR3 having an amino acid sequence of SEQ ID NO:237; or
(i) a VH comprising a vhCDR1 having an amino acid sequence of SEQ ID NO:239; a vhCDR2 having an amino acid sequence of SEQ ID NO:240; and a vhCDR3 having an amino acid sequence of SEQ ID NO:241; and (ii) a VL comprising a vlCDR1 having an amino acid sequence of SEQ ID NO:243, a vlCDR2 having an amino acid sequence of SEQ ID NO: 244, and a vlCDR3 having an amino acid sequence of SEQ ID NO:245; or
(i) a VH comprising a vhCDR1 having an amino acid sequence of SEQ ID NO:247; a vhCDR2 having an amino acid sequence of SEQ ID NO:248; and a vhCDR3 having an amino acid sequence of SEQ ID NO:249; and (ii) a VL comprising a vlCDR1 having an amino acid sequence of SEQ ID NO:251, a vlCDR2 having an amino acid sequence of SEQ ID NO: 252, and a vlCDR3 having an amino acid sequence of SEQ ID NO:253; or
(i) a VH comprising a vhCDR1 having an amino acid sequence of SEQ ID NO:255; a vhCDR2 having an amino acid sequence of SEQ ID NO:256; and a vhCDR3 having an amino acid sequence of SEQ ID NO:257; and (ii) a VL comprising a vlCDR1 having an amino acid sequence of SEQ ID NO:259, a vlCDR2 having an amino acid sequence of SEQ ID NO: 260, and a vlCDR3 having an amino acid sequence of SEQ ID NO:261; or
(i) a VH comprising a vhCDR1 having an amino acid sequence of SEQ ID NO:263; a vhCDR2 having an amino acid sequence of SEQ ID NO:264; and a vhCDR3 having an amino acid sequence of SEQ ID NO:265; and (ii) a VL comprising a vlCDR1 having an amino acid sequence of SEQ ID NO:267, a vlCDR2 having an amino acid sequence of SEQ ID NO: 268, and a vlCDR3 having an amino acid sequence of SEQ ID NO:269; or
(i) a VH comprising a vhCDR1 having an amino acid sequence of SEQ ID NO:271; a vhCDR2 having an amino acid sequence of SEQ ID NO:212; and a vhCDR3 having an amino acid sequence of SEQ ID NO:273; and (ii) a VL comprising a vlCDR1 having an amino acid sequence of SEQ ID NO:275, a vlCDR2 having an amino acid sequence of SEQ ID NO: 276, and a vlCDR3 having an amino acid sequence of SEQ ID NO:277; or
(i) a VH comprising a vhCDR1 having an amino acid sequence of SEQ ID NO:279; a vhCDR2 having an amino acid sequence of SEQ ID NO:280; and a vhCDR3 having an amino acid sequence of SEQ ID NO:281; and (ii) a VL comprising a vlCDR1 having an amino acid sequence of SEQ ID NO:283, a vlCDR2 having an amino acid sequence of SEQ ID NO: 284, and a vlCDR3 having an amino acid sequence of SEQ ID NO:285; or
(i) a VH comprising a vhCDR1 having an amino acid sequence of SEQ ID NO:287; a vhCDR2 having an amino acid sequence of SEQ ID NO:288; and a vhCDR3 having an amino acid sequence of SEQ ID NO:289; and (ii) a VL comprising a vlCDR1 having an amino acid sequence of SEQ ID NO:291, a vlCDR2 having an amino acid sequence of SEQ ID NO: 292, and a vlCDR3 having an amino acid sequence of SEQ ID NO:293; or
(i) a VH comprising a vhCDR1 having an amino acid sequence of SEQ ID NO:295; a vhCDR2 having an amino acid sequence of SEQ ID NO:296; and a vhCDR3 having an amino acid sequence of SEQ ID NO:297; and (ii) a VL comprising a vlCDR1 having an amino acid sequence of SEQ ID NO:299, a vlCDR2 having an amino acid sequence of SEQ ID NO: 300, and a vlCDR3 having an amino acid sequence of SEQ ID NO:301; or
(i) a VH comprising a vhCDR1 having an amino acid sequence of SEQ ID NO:303; a vhCDR2 having an amino acid sequence of SEQ ID NO:304; and a vhCDR3 having an amino acid sequence of SEQ ID NO:305; and (ii) a VL comprising a vlCDR1 having an amino acid sequence of SEQ ID NO:307, a vlCDR2 having an amino acid sequence of SEQ ID NO: 308, and a vlCDR3 having an amino acid sequence of SEQ ID NO:309; or
(i) a VH comprising a vhCDR1 having an amino acid sequence of SEQ ID NO:311; a vhCDR2 having an amino acid sequence of SEQ ID NO:312; and a vhCDR3 having an amino acid sequence of SEQ ID NO:313; and (ii) a VL comprising a vlCDR1 having an amino acid sequence of SEQ ID NO:315, a vlCDR2 having an amino acid sequence of SEQ ID NO: 316, and a vlCDR3 having an amino acid sequence of SEQ ID NO:317; or
(i) a VH comprising a vhCDR1 having an amino acid sequence of SEQ ID NO:319; a vhCDR2 having an amino acid sequence of SEQ ID NO:320; and a vhCDR3 having an amino acid sequence of SEQ ID NO:321; and (ii) a VL comprising a vlCDR1 having an amino acid sequence of SEQ ID NO:323, a vlCDR2 having an amino acid sequence of SEQ ID NO: 324, and a vlCDR3 having an amino acid sequence of SEQ ID NO:325; or
(i) a VH comprising a vhCDR1 having an amino acid sequence of SEQ ID NO:327; a vhCDR2 having an amino acid sequence of SEQ ID NO:328; and a vhCDR3 having an amino acid sequence of SEQ ID NO:329; and (ii) a VL comprising a vlCDR1 having an amino acid sequence of SEQ ID NO:331, a vlCDR2 having an amino acid sequence of SEQ ID NO: 332, and a vlCDR3 having an amino acid sequence of SEQ ID NO:333; or
(i) a VH comprising a vhCDR1 having an amino acid sequence of SEQ ID NO:335; a vhCDR2 having an amino acid sequence of SEQ ID NO:336; and a vhCDR3 having an amino acid sequence of SEQ ID NO:337; and (ii) a VL comprising a vlCDR1 having an amino acid sequence of SEQ ID NO:339, a vlCDR2 having an amino acid sequence of SEQ ID NO: 340, and a vlCDR3 having an amino acid sequence of SEQ ID NO:341.

In some embodiments of the 1+1 Fab-scFv-Fc format, the anti-PSMA ABD has a VH and VL domain selected from the following:
(i) a VH having an amino acid sequence of SEQ ID NO:206; and (ii) a VL having an amino acid sequence of SEQ ID NO:210; or
(i) a VH having an amino acid sequence of SEQ ID NO:214; and (ii) a VL having an amino acid sequence of SEQ ID NO:218; or
(i) a VH having an amino acid sequence of SEQ ID NO:222; and (ii) a VL having an amino acid sequence of SEQ ID NO:226; or
(i) a VH having an amino acid sequence of SEQ ID NO:230; and (ii) a VL having an amino acid sequence of SEQ ID NO:234; or
(i) a VH having an amino acid sequence of SEQ ID NO:238; and (ii) a VL having an amino acid sequence of SEQ ID NO:242; or
(i) a VH having an amino acid sequence of SEQ ID NO:246; and (ii) a VL having an amino acid sequence of SEQ ID NO:250; or
(i) a VH having an amino acid sequence of SEQ ID NO:254; and (ii) a VL having an amino acid sequence of SEQ ID NO:258; or
(i) a VH having an amino acid sequence of SEQ ID NO:262; and (ii) a VL having an amino acid sequence of SEQ ID NO:266; or
(i) a VH having an amino acid sequence of SEQ ID NO:270; and (ii) a VL having an amino acid sequence of SEQ ID NO:274; or
(i) a VH having an amino acid sequence of SEQ ID NO:278; and (ii) a VL having an amino acid sequence of SEQ ID NO:282; or
(i) a VH having an amino acid sequence of SEQ ID NO:286; and (ii) a VL having an amino acid sequence of SEQ ID NO:290; or
(i) a VH having an amino acid sequence of SEQ ID NO:294; and (ii) a VL having an amino acid sequence of SEQ ID NO:298; or
(i) a VH having an amino acid sequence of SEQ ID NO:302; and (ii) a VL having an amino acid sequence of SEQ ID NO:306; or
(i) a VH having an amino acid sequence of SEQ ID NO:310; and (ii) a VL having an amino acid sequence of SEQ ID NO:314; or
(i) a VH having an amino acid sequence of SEQ ID NO:318; and (ii) a VL having an amino acid sequence of SEQ ID NO:322; or
(i) a VH having an amino acid sequence of SEQ ID NO:326; and (ii) a VL having an amino acid sequence of SEQ ID NO:330; or
(i) a VH having an amino acid sequence of SEQ ID NO:334; and (ii) a VL having an amino acid sequence of SEQ ID NO:338.

In some embodiments, the anti-PSMA x anti-CD28 antibody includes a) a CD28 binding domain comprising:

i) a VH comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; and b) a PSMA binding domain comprising:

i) a VH comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221.

FIGS. 10A-10E shows some exemplary Fc domain sequences that are useful in the 1+1 Fab-scFv-Fc format antibodies. The “monomer 1” sequences depicted in FIGS. 10A-10E typically refer to the Fc domain of the “Fab-Fc heavy chain” and the “monomer 2” sequences refer to the Fc domain of the “scFv-Fc heavy chain.” In addition, FIGS. 12 and 13 provide exemplary CH1-hinge domains, CH1 domains, and hinge domains that can be included in the first or second monomer of the 1+1 Fab-scFv-Fc format. Further, FIG. 14 provides useful CL sequences that can be used with this format.

Exemplary subject anti-PSMA x anti-CD28 antibodies in the 1+1 Fab-scFv-Fc format are provided in FIGS. 29A-29RR and 30A-30L.

In some embodiments, the anti-PSMA x anti-CD28 antibody in the 1+1 Fab-scFv-Fc format is C28PB330 or a variant thereof (see FIGS. 30G and 30H). In some embodiments, the anti-PSMA x anti-CD28 antibody in the 1+1 Fab-scFv-Fc format includes: a) a first monomer having an amino acid sequence of SEQ ID NO:351; b) a second monomer having an amino acid sequence of SEQ ID NO:352; and c) a light chain having an amino acid sequence of SEQ ID NO:353.

In some embodiments, the C28PB330 variant includes a deletion of a C-terminal lysine (−K) or lysine and glycine (−GK) in the first monomer or second monomer. In some embodiments, the anti-PSMA x anti-CD28 antibody in the 1+1 Fab-scFv-Fc format includes: a) a first monomer having an amino acid sequence of SEQ ID NO:354; b) a second monomer having an amino acid sequence of SEQ ID NO:355; and c) a light chain having an amino acid sequence of SEQ ID NO:356. In some embodiments, the anti-PSMA x anti-CD28 antibody in the 1+1 Fab-scFv-Fc format includes: a) a first monomer having an amino acid sequence of SEQ ID NO:357; b) a second monomer having an amino acid sequence of SEQ ID NO:358; and c) a light chain having an amino acid sequence of SEQ ID NO:359.

In some embodiments, the anti-PSMA x anti-CD28 antibody in the 1+1 Fab-scFv-Fc format is C28PB397 or a variant thereof (see FIGS. 30D and 30E). In some embodiments, the anti-PSMA x anti-CD28 antibody in the 1+1 Fab-scFv-Fc format includes: a) a first monomer having an amino acid sequence of SEQ ID NO:342; b) a second monomer having an amino acid sequence of SEQ ID NO:343; and c) a light chain having an amino acid sequence of SEQ ID NO:344.

In some embodiments, the C28PB397 variant includes a deletion of a C-terminal lysine (−K) or lysine and glycine (−GK) in the first monomer or second monomer. In some embodiments, the anti-PSMA x anti-CD28 antibody in the 1+1 Fab-scFv-Fc format includes: a) a first monomer having an amino acid sequence of SEQ ID NO:345; b) a second monomer having an amino acid sequence of SEQ ID NO:346; and c) a light chain having an amino acid sequence of SEQ ID NO:347. In some embodiments, the anti-PSMA x anti-CD28 antibody in the 1+1 Fab-scFv-Fc format includes: a) a first monomer having an amino acid sequence of SEQ ID NO:348; b) a second monomer having an amino acid sequence of SEQ ID NO:349; and c) a light chain having an amino acid sequence of SEQ ID NO:350.

### 2. 2+1 Fab2-scFv-Fc Format

One heterodimeric antibody format that finds particular use in subject anti-CD28 x anti-PSMA antibodies provided herein is the 2+1 Fab2-scFv-Fc format (also referred to as “central-scFv format”) shown in FIG. 24B. This antibody format includes three antigen binding domains: two Fab portions and an scFv that is inserted between the VH-CH1 and CH2-CH3 regions of one of the monomers. In some embodiments of this format, the Fab portions each bind PSMA and the “extra” scFv domain binds CD28. In some embodiments, the 2+1 Fab2-scFv-Fc format antibody is a trivalent antibody.

In some embodiments of the 2+1 Fab2-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]—VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 7. This embodiment further utilizes a common light chain that includes a variable light domain (VL1) and a constant light domain (CL). The common light chain associates with the VH1-CH1 of the first and second monomers to form two identical Fabs. In some embodiments, the identical Fabs each bind PSMA. As for many of the embodiments herein, these constructs can include skew variants, pI variants, ablation variants, additional Fc variants, etc. as desired and described herein.

In some embodiments, the first and second Fc domains of the 2+1 Fab2-scFv-Fc format antibody are variant Fc domains that include heterodimerization skew variants (e.g., a set of amino acid substitutions as shown in FIGS. 3A-3F and 9). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab2-scFv-Fc format antibody provided herein includes a charged scFv linker (including those shown in FIGS. 6A-6B). In some embodiments, the 2+1 Fab2-scFv-Fc format antibody provided herein includes FcRn variants M428L/N434S, wherein numbering is according to EU numbering.

In exemplary embodiments 2+1 Fab2-scFv-Fc format antibody with “Platform X” variants, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)4 charged scFv linker (SEQ ID NO:443). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants

L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In exemplary embodiments, the 2+1 Fab2-scFv-Fc format antibody include “Platform J” amino acid modifications as depicted in FIG. 8. In such embodiments, the first Fc domain comprises amino acid substitutions L234A/L235A/D265S/T366S/L368A/Y407V/H435R/Y436F, and the first Fc domain comprises amino acid substitutions L234A/L235A/D265S/LT366W, wherein numbering is according to EU numbering.

In exemplary embodiments of the 2+1 Fab2-scFv-Fc format antibody with Platform J variants, the first Fc domain includes heterodimerization skew variants T366S/L368A/Y407V and the second Fc domain includes heterodimerization skew variants T366W; each of the first and second variant Fc domains include ablation variants L234A/L235A/D265S; and the second Fc domain includes purification variants H435R/Y436F, wherein numbering is according to EU numbering. In some embodiments, the scFv of the 1+1 Fab-scFv-Fc format antibody provided herein includes a (GKPGS)+charged scFv linker. In some embodiments, the 1+1 Fab-scFv-Fc format antibody provided herein includes FcRn variants M252Y/S254T/T256E, wherein numbering is according to EU numbering. In some embodiments, the scFv of the 2+1 Fab2-scFv-Fc Fc format antibody provided herein includes a charged scFv linker (including those shown in FIGS. 6A-6B).

In exemplary embodiments, the 2+1 Fab2-scFv-Fc format antibody with Platform J variants further includes a “stapled” scFv (e.g., a “stapled” anti-CD28 scFv). Stapled” scFvs that exhibit improved stability and/or reduced aggregation are further described in detail herein. Exemplary staple linkers that are useful for inclusion in such “stapled” scFvs are provided in FIGS. 6A-6B. In exemplary embodiments, the stapled linker is GGGSGGSGGCPPCGGSGG (SEQ ID NO:457).

In some embodiments, the scFv of the second monomer of the 2+1 Fab2-scFv-Fc format antibody is a CD28 binding and the VH1 of the first and second monomer and the VL1 of the common light chain each form binding domains that bind PSMA. Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof: 1A7[CD28]_HIL1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, CD28.3[CD28]_H0L0, hCD28.3[CD28]_HIL1, 5.11A1[CD28]_H0L0, TGN1412_H1L1, 341VL34[CD28]_HIL1, 341VL36[CD28]_HIL1, 281VL4[CD28]_H1L1, HuTN228[CD28]_HIL1, PV1[CD28]_H0L0, m9.3[CD28]_H0L0, hu9.3[CD28]_HIL1, 9G2[CD28]_H0L0, 9G2[CD28]_HIL1, 2F10A3.140[CD28]_H1L1, and TN228[CD28]_H4L2 (FIGS. 15-18C, 21A-21H, and 69A-69C).

In some embodiments of the 2+1 Fab2-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:13; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:31; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:35; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:39; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:43; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:47; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:51; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:55; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:59; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:67; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:90; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:94; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:98; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 102; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 106; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 110; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 114; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 118; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 122; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 126; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 130; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:134; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 138; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 142; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:146; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:150; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 158; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:162; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 166; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:170; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 174; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 178; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 182; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:186; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 190; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 194; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 198; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:202; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:380; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:384; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:396; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:400; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:404; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:408; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:412; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:416; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:420; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:424.

In some embodiments of the 2+1 Fab2-scFv-Fc format, the anti-CD28 ABD has a VH and VL domain selected from the following:
(i) a VH comprising a vhCDR1 having an amino acid sequence of SEQ ID NO:10; a vhCDR2 having an amino acid sequence of SEQ ID NO: 11; and a vhCDR3 having an amino acid sequence of SEQ ID NO: 12; and (ii) a VL comprising a vlCDR1 having an amino acid sequence of SEQ ID NO: 14, a vlCDR2 having an amino acid sequence of SEQ ID NO: 15, and a vlCDR3 having an amino acid sequence of SEQ ID NO:16; or
(i) a VH comprising a vhCDR1 having an amino acid sequence of SEQ ID NO:32; a vhCDR2 having an amino acid sequence of SEQ ID NO:33; and a vhCDR3 having an amino acid sequence of SEQ ID NO:34; and (ii) a VL comprising a vlCDR1 having an amino acid sequence of SEQ ID NO:36, a vlCDR2 having an amino acid sequence of SEQ ID NO: 37, and a vlCDR3 having an amino acid sequence of SEQ ID NO:38; or
i) a VH comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; or
i) a VH comprising a vhCDR1 having an amino acid sequence of SEQ ID NO:48; a vhCDR2 having an amino acid sequence of SEQ ID NO:49; and a vhCDR3 having an amino acid sequence of SEQ ID NO:50; and (ii) a VL comprising a vlCDR1 having an amino acid sequence of SEQ ID NO:52, a vlCDR2 having an amino acid sequence of SEQ ID NO: 53, and a vlCDR3 having an amino acid sequence of SEQ ID NO:54; or
i) a VH comprising a vhCDR1 having an amino acid sequence of SEQ ID NO:56; a vhCDR2 having an amino acid sequence of SEQ ID NO:57; and a vhCDR3 having an amino acid sequence of SEQ ID NO:58; and (ii) a VL comprising a vlCDR1 having an amino acid sequence of SEQ ID NO:60, a vlCDR2 having an amino acid sequence of SEQ ID NO:61, and a vlCDR3 having an amino acid sequence of SEQ ID NO:62; or
i) a VH comprising a vhCDR 1 having an amino acid sequence of SEQ ID NO:64; a vhCDR2 having an amino acid sequence of SEQ ID NO:65; and a vhCDR3 having an amino acid sequence of SEQ ID NO:66; and (ii) a VL comprising a vlCDR1 having an amino acid sequence of SEQ ID NO:68, a vlCDR2 having an amino acid sequence of SEQ ID NO:69, and a vlCDR3 having an amino acid sequence of SEQ ID NO:70; or
i) a VH comprising a vhCDR1 having an amino acid sequence of SEQ ID NO:87; a vhCDR2 having an amino acid sequence of SEQ ID NO:88; and a vhCDR3 having an amino acid sequence of SEQ ID NO:89; and (ii) a VL comprising a vlCDR1 having an amino acid sequence of SEQ ID NO:91, a vlCDR2 having an amino acid sequence of SEQ ID NO:92, and a vlCDR3 having an amino acid sequence of SEQ ID NO:93; or
i) a VH comprising a vhCDR1 having an amino acid sequence of SEQ ID NO:95; a vhCDR2 having an amino acid sequence of SEQ ID NO:96; and a vhCDR3 having an amino acid sequence of SEQ ID NO:97; and (ii) a VL comprising a vlCDR1 having an amino acid sequence of SEQ ID NO:99, a vlCDR2 having an amino acid sequence of SEQ ID NO:100, and a vlCDR3 having an amino acid sequence of SEQ ID NO:101; or
i) a VH comprising a vhCDR1 having an amino acid sequence of SEQ ID NO:103; a vhCDR2 having an amino acid sequence of SEQ ID NO: 104; and a vhCDR3 having an amino acid sequence of SEQ ID NO: 105; and (ii) a VL comprising a vlCDR1 having an amino acid sequence of SEQ ID NO:107, a vlCDR2 having an amino acid sequence of SEQ ID NO: 108, and a vlCDR3 having an amino acid sequence of SEQ ID NO: 109; or
i) a VH comprising a vhCDR1 having an amino acid sequence of SEQ ID NO:111; a vhCDR2 having an amino acid sequence of SEQ ID NO:112; and a vhCDR3 having an amino acid sequence of SEQ ID NO: 113; and (ii) a VL comprising a vlCDR1 having an amino acid sequence of SEQ ID NO:115, a vlCDR2 having an amino acid sequence of SEQ ID NO:116, and a vlCDR3 having an amino acid sequence of SEQ ID NO:117; or
i) a VH comprising a vhCDR1 having an amino acid sequence of SEQ ID NO:119; a vhCDR2 having an amino acid sequence of SEQ ID NO:120; and a vhCDR3 having an amino acid sequence of SEQ ID NO: 121; and (ii) a VL comprising a vlCDR1 having an amino acid sequence of SEQ ID NO:123, a vlCDR2 having an amino acid sequence of SEQ ID NO: 124, and a vlCDR3 having an amino acid sequence of SEQ ID NO:125; or
i) a VH comprising a vhCDR1 having an amino acid sequence of SEQ ID NO:127; a vhCDR2 having an amino acid sequence of SEQ ID NO:128; and a vhCDR3 having an amino acid sequence of SEQ ID NO: 129; and (ii) a VL comprising a vlCDR1 having an amino acid sequence of SEQ ID NO: 131, a vlCDR2 having an amino acid sequence of SEQ ID NO: 132, and a vlCDR3 having an amino acid sequence of SEQ ID NO:133; or
i) a VH comprising a vhCDR1 having an amino acid sequence of SEQ ID NO:135; a vhCDR2 having an amino acid sequence of SEQ ID NO:136; and a vhCDR3 having an amino acid sequence of SEQ ID NO: 137; and (ii) a VL comprising a vlCDR1 having an amino acid sequence of SEQ ID NO: 139, a vlCDR2 having an amino acid sequence of SEQ ID NO: 140, and a vlCDR3 having an amino acid sequence of SEQ ID NO:141; or
i) a VH comprising a vhCDR1 having an amino acid sequence of SEQ ID NO:143; a vhCDR2 having an amino acid sequence of SEQ ID NO:144; and a vhCDR3 having an amino acid sequence of SEQ ID NO:115; and (ii) a VL comprising a vlCDR1 having an amino acid sequence of SEQ ID NO:147, a vlCDR2 having an amino acid sequence of SEQ ID NO: 149, and a vlCDR3 having an amino acid sequence of SEQ ID NO:149; or
i) a VH comprising a vhCDR1 having an amino acid sequence of SEQ ID NO:151; a vhCDR2 having an amino acid sequence of SEQ ID NO:152; and a vhCDR3 having an amino acid sequence of SEQ ID NO:153; and (ii) a VL comprising a vlCDR1 having an amino acid sequence of SEQ ID NO:155, a vlCDR2 having an amino acid sequence of SEQ ID NO: 156, and a vlCDR3 having an amino acid sequence of SEQ ID NO:157; or
i) a VH comprising a vhCDR1 having an amino acid sequence of SEQ ID NO:159; a vhCDR2 having an amino acid sequence of SEQ ID NO:160; and a vhCDR3 having an amino acid sequence of SEQ ID NO: 161; and (ii) a VL comprising a vlCDR1 having an amino acid sequence of SEQ ID NO:163, a vlCDR2 having an amino acid sequence of SEQ ID NO: 164, and a vlCDR3 having an amino acid sequence of SEQ ID NO:165; or
i) a VH comprising a vhCDR1 having an amino acid sequence of SEQ ID NO:167; a vhCDR2 having an amino acid sequence of SEQ ID NO:168; and a vhCDR3 having an amino acid sequence of SEQ ID NO: 169; and (ii) a VL comprising a vlCDR1 having an amino acid sequence of SEQ ID NO:171, a vlCDR2 having an amino acid sequence of SEQ ID NO: 172, and a vlCDR3 having an amino acid sequence of SEQ ID NO:173; or
i) a VH comprising a vhCDR1 having an amino acid sequence of SEQ ID NO:175; a vhCDR2 having an amino acid sequence of SEQ ID NO:176; and a vhCDR3 having an amino acid sequence of SEQ ID NO:177; and (ii) a VL comprising a vlCDR1 having an amino acid sequence of SEQ ID NO: 179, a vlCDR2 having an amino acid sequence of SEQ ID NO: 180, and a vlCDR3 having an amino acid sequence of SEQ ID NO:181; or
i) a VH comprising a vhCDR1 having an amino acid sequence of SEQ ID NO:183; a vhCDR2 having an amino acid sequence of SEQ ID NO:184; and a vhCDR3 having an amino acid sequence of SEQ ID NO:185; and (ii) a VL comprising a vlCDR1 having an amino acid sequence of SEQ ID NO:187, a vlCDR2 having an amino acid sequence of SEQ ID NO:188, and a vlCDR3 having an amino acid sequence of SEQ ID NO:189; or
i) a VH comprising a vhCDR1 having an amino acid sequence of SEQ ID NO:191; a vhCDR2 having an amino acid sequence of SEQ ID NO: 192; and a vhCDR3 having an amino acid sequence of SEQ ID NO: 193; and (ii) a VL comprising a vlCDR1 having an amino acid sequence of SEQ ID NO:195, a vlCDR2 having an amino acid sequence of SEQ ID NO: 196, and a vlCDR3 having an amino acid sequence of SEQ ID NO:197; or
i) a VH comprising a vhCDR1 having an amino acid sequence of SEQ ID NO:199; a vhCDR2 having an amino acid sequence of SEQ ID NO:200; and a vhCDR3 having an amino acid sequence of SEQ ID NO:201; and (ii) a VL comprising a vlCDR1 having an amino acid sequence of SEQ ID NO:203, a vlCDR2 having an amino acid sequence of SEQ ID NO:204, and a vlCDR3 having an amino acid sequence of SEQ ID NO:205; or
i) a VH comprising a vhCDR1 having an amino acid sequence of SEQ ID NO:381; a vhCDR2 having an amino acid sequence of SEQ ID NO:382; and a vhCDR3 having an amino acid sequence of SEQ ID NO:383; and (ii) a VL comprising a vlCDR1 having an amino acid sequence of SEQ ID NO:385, a vlCDR2 having an amino acid sequence of SEQ ID NO:386, and a vlCDR3 having an amino acid sequence of SEQ ID NO:387; or
i) a VH comprising a vhCDR1 having an amino acid sequence of SEQ ID NO:389; a vhCDR2 having an amino acid sequence of SEQ ID NO:390; and a vhCDR3 having an amino acid sequence of SEQ ID NO:391; and (ii) a VL comprising a vlCDR1 having an amino acid sequence of SEQ ID NO:393, a vlCDR2 having an amino acid sequence of SEQ ID NO:394, and a vlCDR3 having an amino acid sequence of SEQ ID NO:395; or
i) a VH comprising a vhCDR1 having an amino acid sequence of SEQ ID NO:397; a vhCDR2 having an amino acid sequence of SEQ ID NO:398; and a vhCDR3 having an amino acid sequence of SEQ ID NO:399; and (ii) a VL comprising a vlCDR1 having an amino acid sequence of SEQ ID NO:401, a vlCDR2 having an amino acid sequence of SEQ ID NO:402, and a vlCDR3 having an amino acid sequence of SEQ ID NO:403; or
i) a VH comprising a vhCDR1 having an amino acid sequence of SEQ ID NO:405; a vhCDR2 having an amino acid sequence of SEQ ID NO:406; and a vhCDR3 having an amino acid sequence of SEQ ID NO:407; and (ii) a VL comprising a vlCDR1 having an amino acid sequence of SEQ ID NO:409, a vlCDR2 having an amino acid sequence of SEQ ID NO:410, and a vlCDR3 having an amino acid sequence of SEQ ID NO:411; or
i) a VH comprising a vhCDR1 having an amino acid sequence of SEQ ID NO:413; a vhCDR2 having an amino acid sequence of SEQ ID NO:414; and a vhCDR3 having an amino acid sequence of SEQ ID NO:415; and (ii) a VL comprising a vlCDR1 having an amino acid sequence of SEQ ID NO:417, a vlCDR2 having an amino acid sequence of SEQ ID NO:418, and a vlCDR3 having an amino acid sequence of SEQ ID NO:419; or
i) a VH comprising a vhCDR1 having an amino acid sequence of SEQ ID NO:421; a vhCDR2 having an amino acid sequence of SEQ ID NO:422; and a vhCDR3 having an amino acid sequence of SEQ ID NO:423; and (ii) a VL comprising a vlCDR1 having an amino acid sequence of SEQ ID NO:425, a vlCDR2 having an amino acid sequence of SEQ ID NO:426, and a vlCDR3 having an amino acid sequence of SEQ ID NO:427.

In some embodiments of the 2+1 Fab2-scFv-Fc format, the anti-CD28 ABD has a VH and VL domain selected from the following:
(i) a VH having an amino acid sequence of SEQ ID NO:9; and (ii) a VL having an amino acid sequence of SEQ ID NO:13; or
(i) a VH having an amino acid sequence of SEQ ID NO:31; and (ii) a VL having an amino acid sequence of SEQ ID NO:35; or
(i) a VH having an amino acid sequence of SEQ ID NO:39; and (ii) a VL having an amino acid sequence of SEQ ID NO:43; or
(i) a VH having an amino acid sequence of SEQ ID NO:47; and (ii) a VL having an amino acid sequence of SEQ ID NO:51; or
(i) a VH having an amino acid sequence of SEQ ID NO:55; and (ii) a VL having an amino acid sequence of SEQ ID NO:59; or
(i) a VH having an amino acid sequence of SEQ ID NO:63; and (ii) a VL having an amino acid sequence of SEQ ID NO:67; or
(i) a VH having an amino acid sequence of SEQ ID NO:86; and (ii) a VL having an amino acid sequence of SEQ ID NO:90; or
(i) a VH having an amino acid sequence of SEQ ID NO:94; and (ii) a VL having an amino acid sequence of SEQ ID NO:98; or
(i) a VH having an amino acid sequence of SEQ ID NO:102; and (ii) a VL having an amino acid sequence of SEQ ID NO:106; or
(i) a VH having an amino acid sequence of SEQ ID NO:110; and (ii) a VL having an amino acid sequence of SEQ ID NO:114; or
(i) a VH having an amino acid sequence of SEQ ID NO:118; and (ii) a VL having an amino acid sequence of SEQ ID NO:122; or
(i) a VH having an amino acid sequence of SEQ ID NO:126; and (ii) a VL having an amino acid sequence of SEQ ID NO:130; or
(i) a VH having an amino acid sequence of SEQ ID NO:134; and (ii) a VL having an amino acid sequence of SEQ ID NO:138; or
(i) a VH having an amino acid sequence of SEQ ID NO:142; and (ii) a VL having an amino acid sequence of SEQ ID NO: 146; or
(i) a VH having an amino acid sequence of SEQ ID NO:150; and (ii) a VL having an amino acid sequence of SEQ ID NO:154; or
(i) a VH having an amino acid sequence of SEQ ID NO:158; and (ii) a VL having an amino acid sequence of SEQ ID NO:162; or
(i) a VH having an amino acid sequence of SEQ ID NO:166; and (ii) a VL having an amino acid sequence of SEQ ID NO:170; or
(i) a VH having an amino acid sequence of SEQ ID NO:174; and (ii) a VL having an amino acid sequence of SEQ ID NO:178; or
(i) a VH having an amino acid sequence of SEQ ID NO:182; and (ii) a VL having an amino acid sequence of SEQ ID NO: 186; or
(i) a VH having an amino acid sequence of SEQ ID NO:190; and (ii) a VL having an amino acid sequence of SEQ ID NO:194; or
(i) a VH having an amino acid sequence of SEQ ID NO:198; and (ii) a VL having an amino acid sequence of SEQ ID NO:202; or
(i) a VH having an amino acid sequence of SEQ ID NO:380; and (ii) a VL having an amino acid sequence of SEQ ID NO:384; or
(i) a VH having an amino acid sequence of SEQ ID NO:388; and (ii) a VL having an amino acid sequence of SEQ ID NO:392; or
(i) a VH having an amino acid sequence of SEQ ID NO:396; and (ii) a VL having an amino acid sequence of SEQ ID NO:400; or
(i) a VH having an amino acid sequence of SEQ ID NO:404; and (ii) a VL having an amino acid sequence of SEQ ID NO:408; or
(i) a VH having an amino acid sequence of SEQ ID NO:412; and (ii) a VL having an amino acid sequence of SEQ ID NO:416; or
(i) a VH having an amino acid sequence of SEQ ID NO:420; and (ii) a VL having an amino acid sequence of SEQ ID NO:424.

In some embodiments, the VH1 of the first and second monomer and the VL1 of the common light chain of the 2+1 Fab2-scFv-Fc format antibody each form a binding domain that binds PSMA. In some embodiments, the PSMA binding domain is one of the following PSMA binding domains or a variant thereof: A10[PSMA], A10v2[PSMA], DO1[PSMA], DO1v2[PSMA], E07[PSMA], F02[PSMA], A11[PSMA], F07[PSMA], F07v2[PSMA], G02[PSMA], F01[PSMA], F01v2[PSMA], 011A11[PSMA], PSMB896[PSMA], PSMA-H[PSMA], and D7[PSMA] (FIGS. 25A-25E and 26A-26B).

In some embodiments of 2+1 Fab2-scFv-Fc format, the anti-PSMA ABDs each include a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:206; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:210; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:214; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:218; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:222; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:226; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:230; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:234; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:238; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:242; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:246; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:250; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:254; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:258; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:262; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:266; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:270; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:274; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:278; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:282; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:286; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:290; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:294; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:298; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:302; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:306; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:310; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:314; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:318; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:322; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:326; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:330; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:334; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:338.

In some embodiments of 2+1 Fab2-scFv-Fc format, the anti-PSMA ABDs each include a VH and VL domain selected from the following:
(i) a VH comprising a vhCDR1 having an amino acid sequence of SEQ ID NO:207; a vhCDR2 having an amino acid sequence of SEQ ID NO:208; and a vhCDR3 having an amino acid sequence of SEQ ID NO:209; and (ii) a VL comprising a vlCDR1 having an amino acid sequence of SEQ ID NO:211, a vlCDR2 having an amino acid sequence of SEQ ID NO: 212, and a vlCDR3 having an amino acid sequence of SEQ ID NO:213; or
(i) a VH comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221;
(i) a VH comprising a vhCDR1 having an amino acid sequence of SEQ ID NO:223; a vhCDR2 having an amino acid sequence of SEQ ID NO:224; and a vhCDR3 having an amino acid sequence of SEQ ID NO:225; and (ii) a VL comprising a vlCDR1 having an amino acid sequence of SEQ ID NO:227, a vlCDR2 having an amino acid sequence of SEQ ID NO: 228, and a vlCDR3 having an amino acid sequence of SEQ ID NO:229; or
(i) a VH comprising a vhCDR1 having an amino acid sequence of SEQ ID NO:231; a vhCDR2 having an amino acid sequence of SEQ ID NO:232; and a vhCDR3 having an amino acid sequence of SEQ ID NO:233; and (ii) a VL comprising a vlCDR1 having an amino acid sequence of SEQ ID NO:235, a vlCDR2 having an amino acid sequence of SEQ ID NO: 236, and a vlCDR3 having an amino acid sequence of SEQ ID NO:237; or
(i) a VH comprising a vhCDR1 having an amino acid sequence of SEQ ID NO:239; a vhCDR2 having an amino acid sequence of SEQ ID NO:240; and a vhCDR3 having an amino acid sequence of SEQ ID NO:241; and (ii) a VL comprising a vlCDR1 having an amino acid sequence of SEQ ID NO:243, a vlCDR2 having an amino acid sequence of SEQ ID NO: 244, and a vlCDR3 having an amino acid sequence of SEQ ID NO:245; or
(i) a VH comprising a vhCDR1 having an amino acid sequence of SEQ ID NO:247; a vhCDR2 having an amino acid sequence of SEQ ID NO:248; and a vhCDR3 having an amino acid sequence of SEQ ID NO:249; and (ii) a VL comprising a vlCDR1 having an amino acid sequence of SEQ ID NO:251, a vlCDR2 having an amino acid sequence of SEQ ID NO: 252, and a vlCDR3 having an amino acid sequence of SEQ ID NO:253; or
(i) a VH comprising a vhCDR1 having an amino acid sequence of SEQ ID NO:255; a vhCDR2 having an amino acid sequence of SEQ ID NO:256; and a vhCDR3 having an amino acid sequence of SEQ ID NO:257; and (ii) a VL comprising a vlCDR1 having an amino acid sequence of SEQ ID NO:259, a vlCDR2 having an amino acid sequence of SEQ ID NO: 260, and a vlCDR3 having an amino acid sequence of SEQ ID NO:261; or
(i) a VH comprising a vhCDR1 having an amino acid sequence of SEQ ID NO:263; a vhCDR2 having an amino acid sequence of SEQ ID NO:264; and a vhCDR3 having an amino acid sequence of SEQ ID NO:265; and (ii) a VL comprising a vlCDR1 having an amino acid sequence of SEQ ID NO:267, a vlCDR2 having an amino acid sequence of SEQ ID NO: 268, and a vlCDR3 having an amino acid sequence of SEQ ID NO:269; or
(i) a VH comprising a vhCDR1 having an amino acid sequence of SEQ ID NO:271; a vhCDR2 having an amino acid sequence of SEQ ID NO:212; and a vhCDR3 having an amino acid sequence of SEQ ID NO:273; and (ii) a VL comprising a vlCDR1 having an amino acid sequence of SEQ ID NO:275, a vlCDR2 having an amino acid sequence of SEQ ID NO: 276, and a vlCDR3 having an amino acid sequence of SEQ ID NO:277; or
(i) a VH comprising a vhCDR1 having an amino acid sequence of SEQ ID NO:279; a vhCDR2 having an amino acid sequence of SEQ ID NO:280; and a vhCDR3 having an amino acid sequence of SEQ ID NO:281; and (ii) a VL comprising a vlCDR1 having an amino acid sequence of SEQ ID NO:283, a vlCDR2 having an amino acid sequence of SEQ ID NO: 284, and a vlCDR3 having an amino acid sequence of SEQ ID NO:285; or
(i) a VH comprising a vhCDR1 having an amino acid sequence of SEQ ID NO:287; a vhCDR2 having an amino acid sequence of SEQ ID NO:288; and a vhCDR3 having an amino acid sequence of SEQ ID NO:289; and (ii) a VL comprising a vlCDR1 having an amino acid sequence of SEQ ID NO:291, a vlCDR2 having an amino acid sequence of SEQ ID NO: 292, and a vlCDR3 having an amino acid sequence of SEQ ID NO:293; or
(i) a VH comprising a vhCDR1 having an amino acid sequence of SEQ ID NO:295; a vhCDR2 having an amino acid sequence of SEQ ID NO:296; and a vhCDR3 having an amino acid sequence of SEQ ID NO:297; and (ii) a VL comprising a vlCDR1 having an amino acid sequence of SEQ ID NO:299, a vlCDR2 having an amino acid sequence of SEQ ID NO: 300, and a vlCDR3 having an amino acid sequence of SEQ ID NO:301; or
(i) a VH comprising a vhCDR1 having an amino acid sequence of SEQ ID NO:303; a vhCDR2 having an amino acid sequence of SEQ ID NO:304; and a vhCDR3 having an amino acid sequence of SEQ ID NO:305; and (ii) a VL comprising a vlCDR1 having an amino acid sequence of SEQ ID NO:307, a vlCDR2 having an amino acid sequence of SEQ ID NO: 308, and a vlCDR3 having an amino acid sequence of SEQ ID NO:309; or
(i) a VH comprising a vhCDR1 having an amino acid sequence of SEQ ID NO:311; a vhCDR2 having an amino acid sequence of SEQ ID NO:312; and a vhCDR3 having an amino acid sequence of SEQ ID NO:313; and (ii) a VL comprising a vlCDR1 having an amino acid sequence of SEQ ID NO:315, a vlCDR2 having an amino acid sequence of SEQ ID NO: 316, and a vlCDR3 having an amino acid sequence of SEQ ID NO:317; or
(i) a VH comprising a vhCDR1 having an amino acid sequence of SEQ ID NO:319; a vhCDR2 having an amino acid sequence of SEQ ID NO:320; and a vhCDR3 having an amino acid sequence of SEQ ID NO:321; and (ii) a VL comprising a vlCDR1 having an amino acid sequence of SEQ ID NO:323, a vlCDR2 having an amino acid sequence of SEQ ID NO: 324, and a vlCDR3 having an amino acid sequence of SEQ ID NO:325; or
(i) a VH comprising a vhCDR1 having an amino acid sequence of SEQ ID NO:327; a vhCDR2 having an amino acid sequence of SEQ ID NO:328; and a vhCDR3 having an amino acid sequence of SEQ ID NO:329; and (ii) a VL comprising a vlCDR1 having an amino acid sequence of SEQ ID NO:331, a vlCDR2 having an amino acid sequence of SEQ ID NO: 332, and a vlCDR3 having an amino acid sequence of SEQ ID NO:333; or
(i) a VH comprising a vhCDR1 having an amino acid sequence of SEQ ID NO:335; a vhCDR2 having an amino acid sequence of SEQ ID NO:336; and a vhCDR3 having an amino acid sequence of SEQ ID NO:337; and (ii) a VL comprising a vlCDR1 having an amino acid sequence of SEQ ID NO:339, a vlCDR2 having an amino acid sequence of SEQ ID NO: 340, and a vlCDR3 having an amino acid sequence of SEQ ID NO:341.

In some embodiments of 2+1 Fab2-scFv-Fc format, the anti-PSMA ABDs each include a VH and VL domain selected from the following:
(i) a VH having an amino acid sequence of SEQ ID NO:206; and (ii) a VL having an amino acid sequence of SEQ ID NO:210; or
(i) a VH having an amino acid sequence of SEQ ID NO:214; and (ii) a VL having an amino acid sequence of SEQ ID NO:218; or
(i) a VH having an amino acid sequence of SEQ ID NO:222; and (ii) a VL having an amino acid sequence of SEQ ID NO:226; or
(i) a VH having an amino acid sequence of SEQ ID NO:230; and (ii) a VL having an amino acid sequence of SEQ ID NO:234; or
(i) a VH having an amino acid sequence of SEQ ID NO:238; and (ii) a VL having an amino acid sequence of SEQ ID NO:242; or
(i) a VH having an amino acid sequence of SEQ ID NO:246; and (ii) a VL having an amino acid sequence of SEQ ID NO:250; or
(i) a VH having an amino acid sequence of SEQ ID NO:254; and (ii) a VL having an amino acid sequence of SEQ ID NO:258; or
(i) a VH having an amino acid sequence of SEQ ID NO:262; and (ii) a VL having an amino acid sequence of SEQ ID NO:266; or
(i) a VH having an amino acid sequence of SEQ ID NO:270; and (ii) a VL having an amino acid sequence of SEQ ID NO:274; or
(i) a VH having an amino acid sequence of SEQ ID NO:278; and (ii) a VL having an amino acid sequence of SEQ ID NO:282; or
(i) a VH having an amino acid sequence of SEQ ID NO:286; and (ii) a VL having an amino acid sequence of SEQ ID NO:290; or
(i) a VH having an amino acid sequence of SEQ ID NO:294; and (ii) a VL having an amino acid sequence of SEQ ID NO:298; or
(i) a VH having an amino acid sequence of SEQ ID NO:302; and (ii) a VL having an amino acid sequence of SEQ ID NO:306; or
(i) a VH having an amino acid sequence of SEQ ID NO:310; and (ii) a VL having an amino acid sequence of SEQ ID NO:314; or
(i) a VH having an amino acid sequence of SEQ ID NO:318; and (ii) a VL having an amino acid sequence of SEQ ID NO:322; or
(i) a VH having an amino acid sequence of SEQ ID NO:326; and (ii) a VL having an amino acid sequence of SEQ ID NO:330; or
(i) a VH having an amino acid sequence of SEQ ID NO:334; and (ii) a VL having an amino acid sequence of SEQ ID NO:338.

In some embodiments, the anti-PSMA x anti-CD28 antibody includes a) a CD28 binding domain comprising:

i) a VH comprising a vhCDR1 having an amino acid sequence of SEQ ID NO:40; a vhCDR2 having an amino acid sequence of SEQ ID NO:41; and a vhCDR3 having an amino acid sequence of SEQ ID NO:42; and (ii) a VL comprising a vlCDR1 having an amino acid sequence of SEQ ID NO:44, a vlCDR2 having an amino acid sequence of SEQ ID NO: 45, and a vlCDR3 having an amino acid sequence of SEQ ID NO:46; and b) two PSMA binding domains, each comprising:

i) a VH comprising a vhCDR1 having an amino acid sequence of SEQ ID NO:215; a vhCDR2 having an amino acid sequence of SEQ ID NO:216; and a vhCDR3 having an amino acid sequence of SEQ ID NO:217; and (ii) a VL comprising a vlCDR1 having an amino acid sequence of SEQ ID NO:219, a vlCDR2 having an amino acid sequence of SEQ ID NO: 220, and a vlCDR3 having an amino acid sequence of SEQ ID NO:221.

FIG. 11 shows some exemplary Fc domain sequences that are useful with the 2+1 Fab2-scFv-Fc format. The “monomer 1” sequences depicted in FIG. 11 typically refer to the Fc domain of the “Fab-Fc heavy chain” and the “monomer 2” sequences refer to the Fc domain of the “Fab-scFv-Fc heavy chain.” In addition, FIGS. 12 and 13 provide exemplary CH1-hinge domains, CH1 domains, and hinge domains that can be included in the first or second monomer of the 2+1 Fab2-scFv-Fc format. Further, FIG. 14 provides useful CL sequences that can be used with this format.

### 3. 1+1 CLC Format

One heterodimeric antibody format that finds particular use in subject anti-CD28 x anti-PSMA antibodies provided herein is the “1+1 Common Light Chain” or “1+1 CLC” format, which is depicted in FIG. 24C. The 1+1 CLC format antibody includes a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In such embodiments, the VL pairs with the VH1 to form a first binding domain with a first antigen binding specificity; and the VL pairs with the VH2 to form a second binding domain with a second antigen binding specificity. In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in FIGS. 3A-3F and 9). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering.

In some embodiments, the CH1-hinge-CH2-CH3 of the first monomer comprises amino acid variants

L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the 1+1 CLC format antibody provided herein further includes FcRn variants M428L/N434S, wherein numbering is according to EU numbering.

In some embodiments, one of the first binding domain or the second binding domain binds CD28 and the other binding domain binds PSMA. Any suitable CD28 binding domain and PSMA domain can be included in subject 1+1 CLC format antibody, including any of the CD28 binding domains and PSMA binding domains provided herein or a variant thereof (sec, e.g., FIGS. 15-18C, 21A-21H, 25A-25E and 26A-26B).

### 4. 2+1 CLC Format

Another heterodimeric antibody format that finds particular use in subject CD28 x anti-PSMA antibodies provided herein is the “2+1 Common Light Chain” or “2+1 CLC” format, which is depicted in FIG. 24D. The 2+1 CLC format includes a first monomer that includes a VH1-CH1-linker-VH1-CH1-hinge-CH2-CH3, wherein the VH1s are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. The VL pairs with each of the VH1s of the first monomer to form two first binding domains, each with a first antigen binding specificity; and the VL pairs with the VH2 to form a second binding domain with a second antigen binding specificity. The linker of the first monomer can be any suitable linker, including any one of the domain linkers or combinations thereof described in FIG. 7. In some embodiments, the linker is EPKSCGKPGSGKPGS (SEQ ID NO:483). In some embodiments, the 2+1 CLC format antibody is a trivalent antibody.

In some embodiments, the first and second Fc domains of the 2+1 CLC format are variant Fc domains that include heterodimerization skew variants (e.g., a set of amino acid substitutions as shown in FIGS. 3A-3F and 9). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267 K, and the first Fc domain comprises amino acid variants S364K/E357Q/E233P/L234V/L235A/G236del/S267K, wherein numbering is according to EU numbering.

In some embodiments, each of the two first binding domains binds a tumor PSMA and the second binding domain binds CD28 (see FIGS. 34A-34B). Any suitable CD28 binding domain and PSMA domain can be included in the subject 2+1 CLC format antibody, including any of the CD28 binding domains and PSMA binding domains provided herein or a variant thereof (sec, e.g., FIGS. 15-18C, 21A-21H, 25A-25E and 26A-26B).

5. 2+1 mAb-scFv Format

One heterodimeric antibody format that finds particular use in the subject bispecific anti-CD28 x anti-PSMA antibodies is the 2+1 mAb-scFv format shown in FIG. 24E. This antibody format includes three antigen binding domains: two Fab portions and an scFv that is attached to the C-terminal of one of the heavy chains. In some embodiments of this format, the Fab portions each bind PSMA, in this case, human PSMA and the “extra” scFv domain binds CD28. That is, this mAb-scFv format is a trivalent antibody.

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In these embodiments, the VH1-VL1 each form a first ABD and the VH2-VL2 form a second ABD. In some embodiments, the first ABD binds to a tumor target antigen, including human PSMA, and the second ABD binds human CD28.

In some embodiments, the first and second Fc domains of the 2+1 mAb-scFv format antibody are variant Fc domains that include heterodimerization skew variants (e.g., a set of amino acid substitutions as shown in FIGS. 3A-3F and 9). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIGS. 6A-6B). In some embodiments, the 2+1 mAb-scFv format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)4 charged scFv linker (SEQ ID NO:443). In some embodiments, 2+1 mAb-scFv format antibody provided herein includes FcRn variants M428L/N434S, wherein numbering is according to EU numbering.

In some embodiments, the scFv of the second monomer of the 2+1 Fab2-scFv-Fc format antibody is a CD28 binding and the VH1 of the first and second monomer and the VL1 of the common light chain each form binding domains that bind PSMA. Any suitable CD28 binding domain and PSMA binding domain can be included in the 2+1 mAb-scFv format antibody, including any of the CD28 binding domains and PSMA binding domains provided herein or a variant thereof (see, e.g., FIGS. 15-18C, 21A-21H, 25A-25E and 26A-26B).

6. Dual scFv Formats

One heterodimeric antibody format that finds particular use in the subject bispecific anti-CD28 x anti-PSMA antibodies is the dual scFv format, as are known in the art and shown in FIG. 24F. In this embodiment, the heterodimeric bispecific antibody is made up of two scFv-Fc monomers (both in either (vh-scFv linker-v1-[optional domain linker]-CH2-CH3) format or (v1-scFv linker-vh-[optional domain linker]-CH2-CH3) format, or with one monomer in one orientation and the other in the other orientation.

In this case, all ABDs are in the scFv format. Any suitable PSMA binding domain and CD28 binding domain can be included in anti-CD28 x anti-PSMA antibody in the dual scFv format, including any of the PSMA binding domains and CD28 binding domains provided herein.

In addition, the Fc domains of the dual scFv format comprise skew variants (e.g. a set of amino acid substitutions as shown in FIG. 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 5), optionally charged scFv linkers (including those shown in FIGS. 6A-6B) and the heavy chain comprises pI variants (including those shown in FIG. 4).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable PSMA binding domain and CD28 binding domain can be included in the dual scFv format, including any of the PSMA binding domains and CD28 binding domains provided herein or a variant thereof (see, e.g., FIGS. 15-18C, 21A-21H, 25A-25E and 26A-26B).

7. One-Armed scFv-mAb Format

One heterodimeric antibody format that finds particular use in subject anti-CD28 x anti-PSMA antibodies provided herein is the one-armed mAb-scFv format shown in FIG. 24G. This format includes: 1) a first monomer that comprises an “empty” Fc domain; 2) a second monomer that includes a first variable heavy domain (VH), a scFv domain (a second antigen binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen binding domain and the scFv is a second antigen binding domain. In this format, one of the first antigen binding domain and second binding domain binds CD28, and the other antigen binding domain binds PSMA. As for many of the embodiments herein, these constructs include skew variants, pI variants, ablation variants, additional Fc variants, etc. as desired and described herein.

Any suitable PSMA binding domain and CD28 binding domain can be included in the one-armed scFv-mAb antibody format, including any of the PSMA binding domains and CD28 binding domains provided herein or a variant thereof (see, e.g., FIGS. 15-18C, 21A-21H, 25A-25E and 26A-26B).

8. scFv-mAb format

One heterodimeric antibody format that finds particular use in subject anti-CD28 x anti-PSMA antibodies provided herein is the mAb-scFv format shown in FIG. 24H. In this embodiment, the format relies on the use of a N-terminal attachment of a scFv to one of the monomers, thus forming a third antigen binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vh1-scFv linker-vl1-[optional domain linker]—vh2-CH1-hinge-CH2-CH3) or (with the scFv in the opposite orientation) ((v11-scFv linker-vh1-[optional domain linker]-vh2-CH1-hinge-CH2-CH3)). The second monomer comprises a heavy chain VH2-CH1-hinge-CH2-CH3. This embodiment further utilizes a common light chain comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable PSMA binding domain and CD28 binding domain can be included in the scFv-mAb antibody format, including any of the PSMA binding domains and CD28 binding domains provided herein or a variant thereof (see, e.g., FIGS. 15-18C, 21A-21H, 25A-25E and 26A-26B).

### 9. Non-Heterodimeric Bispecific Antibodies

As will be appreciated by those in the art, the anti-CD28 x anti-PSMA antibodies provided herein can also be included in non-heterodimeric bispecific formats (see FIG. 24I). In this format, the anti-CD28 x anti-PSMA includes: 1) a first monomer comprising a VH1-CH1-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen binding domain and VH2 and VL2 form a second antigen binding domain. One of the first or second antigen binding domains binds CD28 and the other antigen binding domain binds PSMA.

Any suitable PSMA binding domain and CD28 binding domain can be included in anti-CD28 x anti-PSMA antibody in the non-heterodimeric bispecific antibody format, including any of the PSMA binding domains and CD28 binding domains provided herein or a variant thereof (see, e.g., FIGS. 15-18C, 21A-21H, 25A-25E and 26A-26B).

### 10. One Armed Central-scFv

One heterodimeric antibody format that finds particular use in subject anti-CD28 x anti-PSMA antibodies provided herein is the one armed central-scFv format shown in FIG. 24J. In this embodiment, one monomer comprises just an Fc domain, while the other monomer includes a Fab domain (a first antigen binding domain), a scFv domain (a second antigen binding domain) and an Fc domain, where the scFv domain is inserted between the Fc domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable PSMA binding domain and CD28 binding domain can be included in the one armed central-scFv format, including any of the PSMA binding domains and CD28 binding domains provided herein.

11. mAb-Fv Format

One heterodimeric antibody format that finds particular use in subject anti-CD28 x anti-PSMA antibodies provided herein is the mAb-Fv format (FIG. 24K). In this embodiment, the format relies on the use of a C-terminal attachment of an “extra” variable heavy domain to one monomer and the C-terminal attachment of an “extra” variable light domain to the other monomer, thus forming a third antigen binding domain (i.e. an “extra” Fv domain), wherein the Fab portions of the two monomers bind CD28 and the “extra” Fv domain binds PSMA.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vh1-CH1-hinge-CH2-CH3-[optional linker]-v12). The second monomer comprises a second variable heavy domain, a second constant heavy domain comprising a second Fc domain, and a third variable heavy domain covalently attached to the C-terminus of the second Fc domain using a domain linker (vh1-CH1-hinge-CH2-CH3-[optional linker]-vh2. This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

In addition, the Fc domains of the mAb-Fv format comprise skew variants (e.g. a set of amino acid substitutions as shown in FIGS. 3A-3F, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 5), optionally charged scFv linkers (including those shown in FIGS. 6A-6B) and the heavy chain comprises pI variants (including those shown in FIG. 4).

Any suitable PSMA binding domain and CD28 binding domain can be included in the mAb-Fv format, including any of the PSMA binding domains and CD28 binding domains provided herein or a variant thereof (see, e.g., FIGS. 15-18C, 21A-21H, 25A-25E and 26A-26B).

### 12. Central-Fv Format

O One heterodimeric antibody format that finds particular use in subject anti-CD28 x anti-PSMA antibodies provided herein is the central-Fv format shown in FIG. 24L. In this embodiment, the format relies on the use of an inserted Fv domain thus forming an “extra” third antigen binding domain, wherein the Fab portions of the two monomers bind PSMA and the “extra” central-Fv domain binds CD28. The Fv domain is inserted between the Fc domain and the CH1-Fv region of the monomers, thus providing a third antigen binding domain, wherein each monomer contains a component of the Fv (e.g. one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vh1-CH1-[optional linker]-v12-hinge-CH2-CH3). The other monomer comprises a first heavy chain comprising a first variable heavy domain, a CH1 domain and Fc domain and an additional variable heavy domain (vh1-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain comprising a variable light domain and a constant light domain, that associates with the heavy chains to form two identical Fabs that each bind PSMA. The additional variable heavy domain and additional variable light domain form an “extra” central Fv that binds CD28. As for many of the embodiments herein, these constructs include skew variants, pI variants, ablation variants, additional Fc variants, etc. as desired and described herein.

Any suitable PSMA binding domain and CD28 binding domain can be included in the central-Fv format, including any of the PSMA binding domains and CD28 binding domains provided herein or a variant thereof (see, e.g., FIGS. 15-18C, 21A-21H, 25A-25E and 26A-26B).

### 13. Trident Format

In some embodiments, the anti-CD28 x anti-PSMA antibodies provided herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 24M. In this embodiment, the Trident format include a “traditional” heavy and light chain (e.g. VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 24M, the second and third ABDs bind the same antigen.

Any suitable PSMA binding domain and CD28 binding domain can be included in the trident format, including any of the PSMA binding domains and CD28 binding domains provided herein or a variant thereof (see, e.g., FIGS. 15-18C, 21A-21H, 25A-25E and 26A-26B).

### V. Nucleic Acids

In another aspect, provided herein are nucleic acid compositions encoding the anti-CD28 x anti-PSMA antibodies provided herein. A nucleic acid composition may refer to one or multiple polynucleotides.

As will be appreciated by those in the art, the nucleic acid compositions will depend on the format and scaffold of the heterodimeric protein. Thus, for example, when the format requires three amino acid sequences, such as for the 1+1 Fab-scFv-Fc or 2+1 Fab2-scFv-Fc formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer: second monomer:light chains (in the case of many of the embodiments herein that have three polypeptides comprising the heterodimeric antibody) in a 1:1:2 ratio, these are not the ratios that give the best results.

The antibodies provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

VI. Biological and Biochemical Functionality of the anti-CD28 x anti-TAA Antibodies

Generally the anti-CD28 x anti-PSMA antibodies described herein are administered to patients with a PSMA-associated cancer (e.g., a prostate cancer), and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### A. Antibody Compositions for In Vivo Administration

Formulations of the anti-CD28 x anti-PSMA antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### VII. Treatments

Once made, the compositions of the invention find use in a number of oncology applications, by treating cancer, generally by enhancing immune responses (e.g., T cell activation and proliferation), particularly when used with anti-cancer therapies such as anti-tumor bispecific antibodies. In some embodiments, the antibodies provided herein enhance immune responses (e.g., T cell activation and proliferation) by providing agonistic co-stimulation of T cells in the microenvironment of tumors expressing PSMA.

In some embodiments, the anti-CD28 x anti-PSMA bispecific antibodies provided herein are administered with an anti-tumor therapy including, for example, anti-tumor-associated antigen (TAA) bispecific antibodies.

### A. Anti-CD28 x Anti-PSMA/Anti-PSMA Bispecific Antibody

In some embodiments, the anti-CD28 x anti-PSMA antibodies provided herein are administered with an anti-PSMA bispecific antibody that is a T-cell engaging bispecific antibody, such as those that bind to human CD3.

In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 27A). In contrast, only the first signal is provided in treatment with CD3 bispecific antibodies that target a tumor-associated antigen (TAA)(i.e., anti-CD3 x anti-PSMA bispecific antibodies).

Without being bound by any particular theory of operation, it is believed that the anti-CD28 x anti-PSMA bispecific antibodies provided herein can enhance the anti-tumor response of an anti-CD3 x anti-PSMA bispecific antibody by CD28 costimulation (see FIG. 27B). Thus, in one aspect, provided herein are methods of methods of treating a PSMA-associated cancer in a patient by administering the patient an anti-CD3 x anti-PSMA bispecific antibody and an anti-CD28 x anti-PSMA bispecific antibody provided herein.

Anti-CD3 x anti-PSMA antibodies that are useful for providing “signal 1” in combination with the subject anti-CD28 x anti-PSMA antibodies provided herein include, for example, those disclosed in FIGS. 28A-28C. Additional suitable anti-CD3 x anti-PSMA antibodies for providing “signal 1” include, but are not limited to: Acapatamab (see, e.g., Kamat et al., Clin Cancer Res. 27(10:2675-2677 (2021)); DKTK CC-1 (see, e.g., EMBO Mol Med 13:e11902 (2021)), TNB-585 (sec, e.g., J Immunother Cancer 9(6):c002488 (2021)); MOR209/ES414 (see, e.g., U.S. Pat. No. 9,782,478); REGN4338 (U.S. Pat. No. 10,179,819); JNJ-63898081(see, e.g., WO2020/212947); CCW702 (see, e.g., WO2017/136659 and Lec et al., Sci Adv. 7(33):cabi8193 (2021)); HPN424 (see, e.g., WO2021/231434); and Pasotuxizumab (sec, e.g., Hummel et al., Immunotherapy 13(2):125-141 (2021), all of the cited references are incorporate by reference herein in entirety and specifically for pertinent teachings relating to the enumerated exemplary anti-CD3 x anti-PSMA antibodies and antibody sequences. In some embodiments, the anti-CD3 x anti-PSMA antibody and the anti-CD28 x anti-PSMA antibody used in combination bind to the same PSMA epitope. In some embodiments, the anti-CD3 x anti-PSMA antibody and the anti-CD28 x anti-PSMA antibody used in combination bind to different PSMA epitopes.

In addition to anti-CD3 x anti-PSMA antibodies, the subject anti-CD28 x anti-PSMA described herein can also be used as a “signal 2” in combination with an anti-CD3 x anti-B7H3 antibody. See Examples 4, 5 and 7. Thus, in another aspect, provided herein are methods of treating a PSMA-associated cancer in a patient by administering the patient an anti-CD3 x anti-B7H3 bispecific antibody and an anti-CD28 x anti-PSMA bispecific antibody provided herein.

In other embodiments, the subject anti-CD28 x anti-PSMA described herein can also be used as a “signal 2” in combination with a CD3 bispecific antibody that targets an alternative prostate antigen. Non-limiting examples of such “signal 1” bispecific antibodies include an anti-CD3 x anti-hK2 bispecific antibody and an anti-CD3 x anti-TMEFF2 bispecific antibody (e.g., as described in US 2021/0040210 and US 2019/0359711, respectively, which are incorporated by reference herein). Thus, in another aspect, provided herein are methods of treating a PSMA-associated cancer in a patient by administering the patient an anti-CD3 x anti-hK2 bispecific antibody and an anti-CD28 x anti-PSMA bispecific antibody provided herein. In another aspect, provided herein are methods of treating a PSMA-associated cancer in a patient by administering the patient an anti-CD3 x anti-TMEFF2 bispecific antibody and an anti-CD28 x anti-PSMA bispecific antibody provided herein.

### B. Administrative Modalities

The antibodies provided herein administered to a subject, in accord with known methods, such as intravenous administration as a bolus or by continuous infusion over a period of time.

### C. Treatment Modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for case of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, 2014/0288275 and WO2014/145806, all of which are expressly incorporated by reference in their entirety and particularly for the techniques outlined therein.

### Example 1: CD28 Binding Domains

Sequences for human, mouse, and cynomolgus CD28 are depicted in FIGS. 1A-1B and are useful for the development of cross-reactive CD28 antigen binding domains for ease of clinical development.

### 1A: Novel CD28 Binding Domains

An approach considered to avoid the superagonism associated with TGN1412 was to generate novel CD28 binding domains having lower affinity binding to CD28 and/or binding to a different CD28 epitope than TGN1412. In one campaign to generate such novel CD28 binding domains, in-house de novo phage libraries were panned against CD28. 1A(a): Phage-derived clone 1A7

It should be noted that this phage library utilized a human germline VL with diversity introduced into the LCDR3. The amino acid sequences for exemplary phage-derived clone 1A7 are depicted in FIG. 15.

The phage-derived clones were formatted as bivalent mAbs to investigate their binding characteristics. Plasmids containing the variable heavy and variable light domains of select clones were constructed by Gibson assembly and subcloned into a pTT5 expression vector containing the coding sequence for the IgG1 constant regions (with E233P/L234V/L235A/G236del/S67K ablation variants). DNA was transfected in HEK293E for expression and resulting bivalent mAbs were purified from the supernatant using protein A chromatography.

Affinity of the phage-derived bivalent mAbs for CD28 was screened using Octet, a BioLayer Interferometry (BLI)-based method. Experimental steps for Octet generally include the following: Immobilization (capture of ligand to a biosensor); Association (dipping of ligand-coated biosensors into wells containing the analyte); and Dissociation (returning of biosensors to well containing buffer). The resulting apparent dissociation constant (K_{Dapp}) are depicted in FIG. 23 for XENP28428 (based on clone 1A7) and additional phage-derived comparators.

Binding of the phage-derived bivalent mAbs to cell-surface CD28 was investigated. Human PBMCs were incubated with indicated concentrations of XENP28428 or comparator phage-derived mAbs for 1 hour at 4ºC. Cells were then then stained with Alexa Fluor® 647 AffiniPure F(ab′)2 Fragment Goat Anti-Human IgG, Fcγ fragment specific secondary antibody (Jackson ImmunoResearch, West Grove, Penn.) for 1 hour at 4° C. and analyzed by flow cytometry. The data (FIG. 23) show that the phage-derived mAbs were able to bind human PBMCs, although with much weaker maximum binding than prior art anti-CD28 mAb HuTN228 (XENP27181, sequences for which are depicted in FIG. 22).

### 1A(b): 1A7 is not Superagonistic

Potential superagonism of XENP34339 was assessed by air-drying per the Stebbings protocol (Stebbings R. et al. 2007). Air-drying of test articles was achieved by drying in a SpeedVac™ for 2 hours at room temperature. Human PBMCs were treated for 24 hours with 10 μg of air-dried XENP28428 (parental αCD28 mAb 1A7), and activity was compared to the superagonist TGN1412 (XENP29154; sequences for which are depicted in FIG. 22) or PBS control. Air-dried TGN1412 promoted IFNγ secretion from unstimulated human PBMC. In comparison, IFNγ level in PBMCs treated with air-dried XENP28428 remained similar to the negative control of PBS (data shown in FIG. 31).

### 1A(c): Engineering 1A7 Affinity Variants

Towards optimization of PSMA x CD28 bsAbs as described in Example 5, numerous 1A7 affinity variants were developed by engineering VH variants (illustrative sequences as depicted in FIG. 16 and additional sequences depicted as SEQ ID NOs: 19-26), VL variants (illustrative sequences as depicted in FIG. 17 and additional sequences depicted as SEQ ID NOs: 27-30), consensus sequences for which are depicted in FIGS. 19A-19B, and combinations thereof (illustrative sequences for which are depicted in FIG. 18). Monovalent affinity of illustrative variants, in the context of scFvs, are depicted in FIG. 20.

### 1B: Additional CD28 Binding Domains

Sequences for additional CD28 binding domains which may find use in the PSMA x CD28 bsAbs of the invention are depicted in FIG. 21A-21H.

### Example 2: PSMA Binding Domains

Sequences for human, mouse, and cynomolgus PSMA are depicted in FIGS. 2A-2B and are useful for the development of cross-reactive PSMA antigen binding domains for case of clinical development.

### 2A: Novel PSMA Binding Domains

As will be described below, the PSMA x CD28 bsAbs of the invention were engineered with the aim to combine with PSMA x CD3 bsAbs. More specifically, they were anticipated to be combined with PSMA x CD3 bsAbs such as 1391 PSMA x CD3 and 1508 PSMA x CD3 (sequences for which are depicted in FIGS. 28A-28C). As will be described in Example 4C, PSMA x CD28 and PSMA x CD3 bsAbs having competing PSMA binding domains do not combine well at increasing concentrations. Therefore, novel PSMA binding domains were generated and selected based on binning to different epitope than 1391 and 1508, sequences for which are depicted in FIGS. 25A-25E. It should be noted that A10v2, DO1v2, F07v2, G02v2, and F01v2 are respectively identical to A10, DO1, F07, G02, and F01, except that degradation liabilities were removed.

### 2B: Additional PSMA Binding Domains

Sequences for additional PSMA binding domains which may find use in the PSMA x CD28 bsAbs of the invention are depicted in FIGS. 26A-26B.

### Example 3: Engineering PSMA x CD28 bsAbs

T cells require multiple signals for complete activation and differentiation. Signal 1, promoted by recognition of a peptide-MHC (pMHC) complex by the T cell receptor (TCR), is absolutely required for T cell activation. Signal 2, which synergizes with, and amplifies Signal 1, is typically provided by the interaction of the CD28 ligands CD80 and CD86 with CD28 itself. Although CD28 engagement alone is typically inert, when combined with Signal 1 activation, it promotes additional activation, survival, and proliferative signals, including IL2 secretion (sec FIGS. 27A-27B). As CD80 and CD86 are only naturally expressed by professional antigen-presenting cells (APC), the extent of CD28 costimulation in the tumor setting can be highly variable. By creating this novel class of tumor-targeted CD28 bispecific antibodies, CD80/CD86 engagement of CD28 can be mimicked, providing an artificial source of Signal 2. Notably, signal can either be provided by the natural TCR:pMHC recognition of tumor cells, or it can be provided by combination of the CD28 bispecific with a CD3 bispecific (which can mimic Signal 1). As there are an increasing number of PSMA x CD3 undergoing clinical development for prostate cancer, it would be useful to develop PSMA x CD28 bsAbs to synergize with such molecules.

A number of formats are contemplated for use, schematics for which are outlined in FIG. 23. One exemplary format utilizing Fab domains and scFv is the 1+1 Fab-scFv-Fc format (depicted schematically in FIGS. 24A-24F) which comprises a first monomer comprising a single-chain Fv (“scFv”) with a first antigen binding specificity covalently attached to a first heterodimeric Fc domain i.e., scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain. Any number of heterodimerization approaches as is known in the art could find use in this (and other) bispecific formats, in combination with any number of approaches for purifying heterodimers from contaminating homodimers, including those depicted in FIGS. 3A-3F. Regardless of bsAb format, the CD28 bispecific antibodies are monovalent for CD28 and incorporate Fc variants engineered to ablate FcγR binding to avoid potential superagonism. Such Fc variants include those depicted in FIG. 5. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs.

Two platforms based on the 1+1 Fab-scFv-Fc format were used, herein referred to as Platform X and Platform Y.

### 3A: Platform X

Platform X (as described in FIG. 8) utilizes Backbones 1 or 11 in FIGS. 10A-10E. Specifically, this Platform utilizes the L368D/K370S (on the HC): S364K/E357Q (on the scFv-Fc) heterodimeric Fc variants. The Fab side further includes pI variants N208D/Q295E/N384D/Q418E/N421D to increase negative charge of the heavy chain. The sc Fv utilizes a positively charged (GKPGS)4 linker (SEQ ID NO:443) between the VH and VL domains to increase positive charge of the scFv-Fc chain. Collectively, these two approaches enable easy purification of heterodimers from contaminating homodimers. The FcγR ablation variants utilized in this platform are the E233P/L234V/L235A/G236/S267K substitutions on both the HC and the scFv-Fc monomers. In some cases, this platform includes the M428L/N434S half-life extension variants. Sequences for illustrative PSMA x CD28 bsAbs (based on binding domains as described in Examples 1 and 2) in the 1+1 Fab-scFv-Fc format and Platform X are depicted in FIGS. 29A-29RR.

### 3B: Platform J

Platform J (as described in FIG. 8) utilizes Backbones 13 or 14 in FIGS. 10A-10E. Specifically, this Platform utilizes the T366S/L368A/Y407V (on the HC): T366W (on the scFv-Fc) heterodimeric Fc variants. The Fab side further includes H435R/Y436F to facilitate purification. The scFv may utilize a GGSEGKSSGSGSESKSTGGS (SEQ ID NO:456) (unstapled) or a GGGSGGSGGCPPCGGSGG (SEQ ID NO:457) (stapled) linker between the VH and VL domains. When using the latter stapled scFv linker, the VH and VL further include cysteine substitutions to form disulfide bridges with the linker. The FcγR ablation variants utilized in this platform are the L234A/L235A/D265S substitutions on both the HC and the scFv-Fc monomers. In some cases, this platform includes the

M252Y/S254T/T256E half-life extension variants. Sequences for illustrative PSMA x CD28 bsAbs (based on binding domains as described in Examples 1 and 2) in the 1+1 Fab-scFv-Fc format and Platform J are depicted in FIGS. 30A-30L.

It should be noted that components from Platform X and Platform J can be mix and matched. For example, Platform J may utilize the (GKPGS)4 linker (SEQ ID NO:443) of Platform X. Additionally, each of Platform X and Platform J may use alternative variants (e.g. alternative scFv linker, alternative half-life extension variants, alternative FcγR ablation variants, etc.).

### Example 4: Prototype PSMA x CD28 bsAbs

Prototype PSMA x CD28 bsAbs were engineered and produced with high and low affinity 1A7 CD28 binding domains, respectively XENP37902 (1A7_H1.14_L1; 230 nM) and XENP37903 (1A7_H1.14_L1.71; 37 nM), sequences for which are depicted in FIGS. 29A-29RR.

4A: PSMA x CD28 bsAbs Combine Productively with CD3 bsAbs

To investigate the combination of PSMA x CD28 bsAbs with CD3 bsAbs, the prototype PSMA x CD28 bsAbs were paired with an illustrative B7H3 x CD3 bsAb or an illustrative PSMA x CD3.

In a first experiment, 2,500 C42B-NLR (PSMA^{hi}) and DU145-NLR (PSMA1º) cancer cells were seeded. After 48 hours, CD3+ T cells were added at an effector to target ratio of 1:1 along with 1 μg/mL B7H3 x CD3 bsAb alone or in combination with 1 μg/mL XENP37902 or XENP37903. Data depicting induction of Redirected T Cell Cytotoxicity (RTCC) and T cell proliferation by the test articles are depicted in FIGS. 32 and 33A-33B. The data show that combination with PSMA x CD28 enhances both RTCC and T cell proliferation in the presence of PSMA^{hi} C42B-NLR cancer cells. Notably, the low affinity 1A7_H1.14_L1 230 nM CD28 binding domain in XENP37902 was sufficient to enhance activity. Further, the data show that while the B7H3 x CD3 bsAb alone induces RTCC and T cell proliferation in the presence of PSMA^{null} DU145-NLR cancer cells, combination with the PSMA x CD28 bsAbs does not enhance activity indicating that PSMA x CD28 bsAbs only enhance activity in the presence of PSMA antigen.

In a second similar experiment, 2,500 C42B-NLR (PSMA^{hi}) and DU145-NLR (PSMA1º) cancer cells were seeded. After 48 hours, CD3+ T cells were added at an effector to target ratio of 1:1 along with 1 μg/mL PSMA x CD3 bsAb alone or in combination with 1 μg/mL XENP37902 or XENP37903. It should be noted that the PSMA x CD3 does not compete with the PSMA x CD28 bsAbs (i.e. their PSMA binding domains bind different epitopes). Data depicting induction of RTCC and T cell proliferation by the test articles are depicted in FIGS. 34A-34B and 35. Consistent with the above, the data show that combination with PSMA x CD28 enhances both RTCC and T cell proliferation in the presence of PSMA^{hi} C42B-NLR cancer cells, but not in the presence of PSMA1^{lo} DU145-NLR cancer cells.

4B: PSMA x CD28 bsAbs are Active on PSMA^{hi} and PSMA^{med} Cells

To further investigate the effect of PSMA antigen density on PSMA x CD28 activity, PC3 cell lines expressing varying PSMA antigen densities were generated. Cell-surface PSMA antigen density levels on the PSMA-transfected PC3 cell lines were estimated by FACS using fluorescently-labeled beads as advised by the QuickCal protocol (Bangs Laboratories, Inc., Fishers, IN). 50,000 cells per well and MESF beads were combined with A647—conjugated anti-PSMA mAh (J591) for 30 minutes at 4° C. Cells were then washed and fixed in 1% PFA. Flow cytometry was performed to determine antibody binding, and antigen density calculations were carried out using QuickCal® V.2.3. software (Bangs Laboratories, Inc., Fishers, IN). PSMA-transfected PC3 cell lines are hereon referred to by their PSMA density (i.e. PC3 (˜100k) has a MESF score of 100,000).

100,00 CD3+ T cells were treated with a dose titration off XENP37902 or XENP37903 in the presence of 100,000 PC3 cancer cells (of varying PSMA densities) and constant 1 μg/mL non-competing PSMA x CD3 bsAb. 1 day post T cell seeding, IL-2 secretion was measured using MSD (Meso Scale Discovery, Rockville, Md.), data for which are depicted in FIG. 36. The data show that the PSMA x CD28 bsAb enhanced cytokine secretion in the presence of both PSMA^{hi} and PSMA^{med} cells, but not in the presence of PSMA1^{lo} cells.

4C: PSMA x CD28 bsAbs do not Combine Well with Competing PSMA x CD3 bsAbs

In the experiments above, a non-competing PSMA x CD3 bsAb was utilized. To investigate whether the PSMA x CD28 bsAbs combine productively with PSMA x CD3 bsAbs binding to the same epitope, additional competing PSMA x CD3 bsAb XENP32220 (sequences depicted in FIGS. 28A-28C) was utilized. 100,00 CD3+ T cells were treated with a dose titration off XENP37902 or XENP37903 in the presence of 100,000 PC3 cancer cells (of varying PSMA densities) and constant 1 μg/mL XENP32220. 1 day post T cell seeding, IL-2 secretion was measured using MSD (Meso Scale Discovery, Rockville, Md.), data for which are depicted in FIG. 37. The data show that while the PSMA x CD28 bsAbs enhanced IL-2 secretion at lower concentration, there was a hooking effect and activity was diminished at higher concentrations. This suggests that it is useful for the PSMA x CD28 and PSMA x CD3 bsAbs to bind different epitopes, especially when higher concentration doses of PSMA x CD28 are expected.

### Example 5: Characterizing PSMA x CD28 bsAbs Utilizing Novel PSMA Binding Domains

Additional PSMA x CD28 bsAbs were engineered in Platform X utilizing the novel PSMA binding domains as described in Example 2A. As noted above in Example 2A, the novel PSMA binding domains utilized in the PSMA x CD28 bsAbs were selected as they bind different PSMA epitope than selected PSMA x CD3 bsAb (1391 and 1508). Additionally for the investigation described in this section, medium CD28 affinity (1A7_H1_L1.71; 180 nM) binding domain was utilized to pull out differences on the PSMA side.

5A: PSMA x CD28 bsAbs Bound PSMA^{hi} and PSMA^{med} Cell Lines

First, the ability of the PSMA x CD28 bsAbs to bind to PSMA⁺ cell lines was investigated. 100,000 22Rv1 and C42B cancer cells were treated with a dose titration of the indicated PSMA x CD28 bsAbs at 37 degrees for 30 minutes. Cells were washed and then stained with anti-human-Ig-FITC for detecting bound bsAbs, data for which are depicted in FIG. 38. The data show that the PSMA x CD28 bsAbs bound the PSMA⁺ cell lines with varying potencies. Notably, A10v2 demonstrated similar binding as A10; and DO1v2 demonstrated similar binding as DO1v2 indicating that the degradation liability fix did not negatively affect their binding. In an additional internalization experiment (data not shown), the bsAbs were found to internalize into C42B at different rates as well. While All bound well to PSMA⁺ cell lines, it internalizes much quicker than the other PSMA binding domain. Additionally, while DO1 and DO1v2 demonstrated similar binding to the PSMA⁺ cell lines, DO1v2 internalizes more rapidly. PSMA binding domains having potent binding and low internalization rates were preferred.

5A: PSMA x CD28 bsAb Demonstrated In Vitro Activity Consistent with their PSMA Binding

Next, the in vitro activity of the PSMA x CD28 bsAbs were investigated. In a first experiment, 10,000 22Rv1-NLR (PSMA^{med}) were seeded. The next day, T cells were added at an effector to target ratio of 1:1 with constant 1 μg/ml B7H3 x CD3 bsAb and dose titration of PSMA x CD28 bsAbs. IL-2 was measured after 24 hours, data for which are depicted in FIG. 39. Next, a similar experiment was performed combining PSMA x CD28 bsAbs with PSMA x CD3 bsAbs 1391 or 1508. As before, IL-2 was measured after 24 hours, data for which are depicted in FIGS. 40 and 56. The data show a range of potencies consistent with cell binding. Notably as with binding, the degradation liability fixed A10v2 and DO1v2 binding domains demonstrated similar activity as their parental counterparts.

### Example 6: Tuning CD28 Binding Affinity

Based on observations associated with other T cell engagers (e.g. CD3 bsAbs), higher binding affinity for T cell antigen may negatively affect pharmacokinetic profile. Accordingly, preferred PSMA binding domains identified in Example 5 were paired with CD28 binding domains having varying CD28 binding affinities to identify the weakest CD28 binding domain that provides sufficient activity.

6A: Characterizing CD28 bsAb by Titrating PSMA x CD3 bsAb (Constant CD28 bsAb)

In a first set of experiment investigating the effect of varying CD28 binding affinities when paired with A10v2 or DO1v2, constant 1 μg/mL dose of CD28 bsAbs were combined with a titration of PSMA x CD3 (1391 or 1508) in the presence of C42B-NLR (PSMA^{hi}), PC3-PSMA (˜100K), 22Rv2-NLR (PSMA^{med}), and PC3-PSMA (˜50K). Experiments were performed by seeding 10,000 cancer cells and incubating with T cells (1:1 E:T) and test articles after 24 hours. After a further 24 hour incubation, IL-2 secretion was measured, data for which are depicted in FIGS. 57 and 40 for A10v2 and FIGS. 42 and 58 for DO1v2. The data show that most of the CD28 binding domains functioned well in the PSMA x CD28 bsAbs based on A10v2 and DO1v2.

6B: Characterizing CD28 bsAb by Titrating PSMA x CD28 bsAb (Constant PSMA x CD3 Bsab)

In a further set of experiments investigating the effect of varying CD28 binding affinities when paired with A10v2 or DO1v2, constant 1 μg/mL dose of PSMA x CD3 (1391 or 1508) were combined with a titration of PSMA x CD28 bsAbs in the presence of C42B-NLR (PSMA^{hi}), PC3-PSMA (˜100K), 22Rv2-NLR (PSMA^{med}), and PC3-PSMA (˜50K). Experiments were performed by seeding 10,000 cancer cells and incubating with T cells (1:1 E:T) and test articles after 24 hours. After a further 24 hour incubation, IL-2 secretion was measured, data for which are depicted in FIGS. 43 and 59 for A10v2 and FIGS. 44 and 60 for DO1v2. Consistent with the above, the CD28 binding domains all functioned well in the PSMA x CD28 bsAbs based on A10v2 and DO1v2.

Next, additional PSMA x CD28 bsAbs based on E07 paired with different CD28 binding affinities were investigated. Constant 1 μg/mL dose of PSMA x CD3 (1391 or 1508) were paired with dose titration of PSMA x CD28 bsAbs (based on A10v2, DO1v2, and E07) in the presence of 22Rv1-NLR (PSMA^{med}) or PSMA-PC3 (˜50K) cells. Experiments were performed by seeding 10,000 or 1,000 cancer cells and incubating with T cells (for 1:1 or 0.1:1 E:T ratio) and test articles after 24 hours. After a further 24 hour incubation, IL-2 secretion was measured, data for which are depicted in FIGS. 45-46 and 61-62. 5 days post incubation with test articles, T cell counts were analyzed using FACS, data for which are depicted in FIG. 47. Consistent with the foregoing, there is a ladder of potencies enabled by the use of the different PSMA binding domains (A10v2 >DO1v2 >E07).

In another experiment, 1,250 22Rv1-NLR cancer cells were seeded. After 48 hours, T cells were added at an 1:1 E:T ratio with constant 1 μg/mL PSMA x CD28 bsAbs and 0.3 ng/mL, 1 ng/mL, 20 ng/ml, or 2.5 μg/mL 1391 PSMA x CD3. Incucyte recorded target cells over a period of 10 days (as an indicator off RTCC), as depicted in FIG. 48. At low 0.3 ng/mL concentration PSMA x CD3 bsAb, the CD28 bsAbs did not noticeably enhance RTCC activity. However at higher 1 ng/ml, 10 ng/ml, and 2.5 μg/mL PSMA x CD3 bsAb, the CD28 bsAbs noticeably enhanced RTCC activity. Notably, at 1 ng/mL PSMA x CD28 bsAbs with high affinity 37 nM and 96 nM CD28 binding domains lagged behind corresponding bsAbs with 180 nM and 230 nM CD28 binding domains. This trend is also observed in the combination with high concentration 2.5 μg/mL PSMA x CD3 bsAb.

### 6C: Investigating Lower CD28 Binding Affinities

In certain contexts (e.g. when targeting tumors with higher antigen density or combining with higher concentration CD3 bsAb), lower affinity CD28 binding domains may be preferred. In view of these observations, lower CD28 binding affinities were also investigated and XENP39231 and XENP40470 were produced pairing A10v2 with 1A7_H1.1_L1 600 nM and 1A7_HIL1 1000 nM CD28 binding domains. 10,000 PC3-PSMA (˜100K) or 22Rv1-NLR (PSMA^{med}) cancer cells were seeded with T cells at a 1:1 E:T ratio in the presence of PSMA x CD3 (1391 or 1508) and dose titration of CD28 bsAbs. IL2 was assessed after 24 hours, data for which are shown in FIGS. 49 and 63. The data show that on PSMA^{hi} cell lines, the lower affinity CD28 binding domains were still functional (although potency correlated to affinity). On the PSMA^{med} cell line, the 600 nM CD28 binding domain was still functional.

### Example 7: Further Characterization of PSMA x CD28 bsAbs

7A: PSMA x CD28 bsAbs in Combination with PSMA x CD3 is Only Active on PSMA Positive Cells Lines

In combining CD3 and CD28 agonistic antibodies, it is especially important to ascertain that there are no off-target toxicities. To confirm this, activity of the PSMA x CD28 and PSMA x CD3 combination in the presence of PSMA^{med} (PSMA-PC3 (˜50K)), PSMA^{lo} (PSMA-PC3 (˜3K)), and PSMA^{null} (DU145-NLR) cell lines were investigated. 10,000 cancer cells per well were seeded. After 24 hours, T cell were added at an effector to target ratio of 1:1 with 1 μg/mL 1391 PSMA x CD3 or 1508 PSMA x CD3 and dose titration off CD28 bsAbs. IL2 was measured after 24 hours, data for which are depicted in FIGS. 50 and 64. The data show that in fact the combination is only active on PSMA⁺ cell lines.

7B: PSMA x CD28 bsAbs Produced on Platform X and Platform J Function Similarly

To investigate differences between Platform X and Platform J, XENP39234, XENP39233, and XENP38936 produced on Platform X were also produced on Platform J (i.e. grafting the variable regions). In a first experiment, binding of the bsAbs to C42B (PSMA^{hi}) cells was determined, data for which are depicted in FIG. 66. 10,000 PC3-PSMA (˜50K) were seeded. After 24 hours, T cells were added at a 1:1 E:T ratio with 1 μg/mL PSMA x CD3 (1391 or 1508) and dose titration of CD28 bsAbs. IL2 was measured after 2 hours, data for which are depicted in FIGS. 51 and 65. The data show that molecules produced on Platform X and Platform J function similarly.

7C: PSMA x CD28 bsAbs Combine More Potently with Tumor Antigen Matched CD3 Bispecifics

As previously noted, the PSMA x CD28 bsAbs of the invention were engineered with the aim to combine with certain PSMA x CD3 bsAbs. Nonetheless, it would be useful for the bsAbs to combine productively with other CD3 bsAbs. 10,000 PC3-PSMA (˜100K) cancer cells were seeded. After 24 hours, T cells were added at 1:1 E:T ratio with 1 μg/mL B7H3 x CD3 or PSMA x CD3 and dose titration of PSMA x CD28 bsAbs. IL2 and IFNγ secretion were measured after 1 day and RTCC was measured for 10 days, data for which are depicted in FIG. 52. The data show that the PSMA x CD28 bsAbs also combine productively with B7H3 x CD3 bsAbs. Nonetheless, they combine more potently with tumor antigen matched CD3 bsAbs.

### Example 8: In Vivo Anti-Tumor Activity

In a first experiment, NSG mice that were MHC EII-DKO (NSG-DKO) and thus resistant to GVHD were used. NSG-DKO mice (10 per group) were intradermally inoculated with 1×10⁶ PC3-PSMA (˜100K) cells on Day −13. Mice were then intraperitoneally injected with 5×10⁶ human PBMCs and treated with the indicated test articles/test article combinations on Day 0, and further treated with the indicated test articles on Days 7 and 14. Tumor volumes were monitored by caliper measurements once to three times per week, data for which are shown (days post 1^{st} dose) in FIGS. 53-54. Blood was drawn weekly and analyzed by flow cytometry to count expansion of human lymphocytes, data for which are shown in FIG. 55. The data show that each of the PSMA x CD28 bsAbs demonstrated anti-tumor activity as a single agent. This is likely by providing co-stimulation to alloreactivity between the human T cells and tumor cells. The PSMA x CD3 and B7H3 x CD3 bsAbs as single agents also enhanced anti-tumor activity. Each of the PSMA x CD28 bsAbs further enhance anti-tumor activity when combined with the CD3 bsAbs (although more pronounced with a weaker Signal 1 e.g. in the case of lower dose PSMA x CD3). Notably, XENP39231 having a weak 1000 nM CD28 binding domain outperformed XENP38936 having a tighter 180 nM CD28 binding domain when combined with PSMA x CD3 bsAb.

### Example 9: IL2 Production of Anti-PSMAxCD28 in Combination with PSMAxCD3 on Target Cells Using Human PBMCs

C42B human prostate tumor cells were washed with DPBS and 0.05% trypsin was added to allow cells to detach. Media was added to neutralize trypsin and the cells were transferred to a 15 mL conical with DPBS. The cells were centrifuged 1200 rpm for 3 minutes. DPBS was aspirated and cells were re-suspended in RPMI-10 complete medium. The cells were counted using the Vi-cell XR cell viability analyzer and were plated at 15K/well in 100 μL RPMI-10 complete medium. The plates were incubated at 37ºC, 5% CO₂ overnight. Human PBMC Cells (Discovery Life Services) were thawed and transferred to a 15 mL conical with DPBS. The cells were centrifuged 1500 rpm for 3 minutes. DPBS was aspirated and cells were re-suspended in RPMI-10 complete medium. The cells were counted using the Vi-cell XR cell viability analyzer and were plated at an E:T of 1:1(normalized to % CD3) in 50 μL of RPMI-10/well. PSMAxCD28 C28PB397 was prepared in a dilution block with a starting concentration of 40 nM, diluted 3-fold for a total of 10 dilution points including an untreated control. PSMAxCD28 C28PB397 was diluted in RPMI −10 or 1 nM of PSMAxCD3 1508. The molecules were added at 50 μL/well in RPMI-10 and plate was placed into an incubator at 37° C. overnight. Supernatant was collected after 24 hr of incubation. IL2 production was measured using IL2 AlphaLISA Kit(Perkinelmer). Data was graphed (GraphPad Prism) using a non-linear fit, log(agonist) vs. response-variable slope (4-parameters) (FIG. 72).

1-159. (canceled)

160. A bispecific antibody comprising a first antigen binding domain (ABD) that binds PSMA and a second ABD that binds CD28, wherein the first ABD that binds PSMA comprises a VH of SEQ ID NO: 214 and a VL of SEQ ID NO: 218, and the second ABD that binds CD28 comprises a VH of SEQ ID NO: 396 and a VL of SEQ ID NO: 400.

161. The bispecific antibody of claim 160, wherein the first ABD or second ABD is an scFv.

162. The bispecific antibody of claim 161, wherein the scFv comprises an scFv linker selected from GKPGSGKPGSGKPGSGKPGS (SEQ ID NO: 443), GGSEGKSSGSGSESKSTGGS (SEQ ID NO: 456), and GGGSGGSGGCPPCGGSGG (SEQ ID NO: 457).

163. The bispecific antibody of claim 162, wherein the second ABD is the scFv, and the scFv linker is SEQ ID NO:457.

164. The bispecific antibody of claim 160, wherein the bispecific antibody further comprises a first Fc domain and a second Fc domain.

165. The bispecific antibody of claim 164, wherein the first Fc domain and the second Fc domain are each variant human IgG1, IgG2, or IgG4 Fc domains.

166. The bispecific antibody of claim 165, wherein one of the first and second Fc domains comprises heterodimerization variant T366W, and the other of the first and second Fc domains comprises heterodimerization variants T366S/L368A/Y407V, wherein numbering is according to EU numbering.

167. The bispecific antibody of claim 165, wherein the first and the second Fc domains each comprise ablation variants L234A/L235A/D265S, wherein numbering is according to EU numbering.

168. The bispecific antibody of claim 165, wherein the first or the second Fc domain comprises purification variants H435R/Y436F, wherein numbering is according to EU numbering.

169. The bispecific antibody of claim 165, wherein the first Fc domain comprises amino acid substitutions L234A/L235A/D265S/LT366W, and the second Fc domain comprises amino acid substitutions L234A/L235A/D265S/T366S/L368A/Y407V/H435R/Y436F, wherein numbering is according to EU numbering.

170. The bispecific antibody of claim 160, wherein the bispecific antibody comprises a first monomer that is at least 95% identical to SEQ ID NO: 342, a second monomer that is at least 95% identical to SEQ ID NO: 343, and a light chain that is at least 95% identical to SEQ ID NO: 344.

171. A pharmaceutical composition comprising the bispecific antibody of claim 160 and a pharmaceutically acceptable carrier.

172. A nucleic acid composition encoding the bispecific antibody of claim 160.

173. An expression vector composition comprising the nucleic acid composition of claim 172.

174. A host cell comprising the expression vector composition of claim 173.

175. A method of making a bispecific antibody comprising culturing the host cell of claim 174 under conditions wherein the bispecific antibody is expressed and recovering the bispecific antibody.

176. A method of treating prostate cancer in a patient in need thereof, comprising administering to the patient the bispecific antibody of claim 160.

177. The method of claim 176, wherein the prostate cancer is castration-resistant prostate cancer.

178. A method of enhancing T cell proliferation in the presence of PSMA-expressing cells, comprising contacting the cells with the bispecific antibody of claim 160.

179. A method of inhibiting the growth or proliferation of PSMA-expressing cells, comprising contacting the cells with the bispecific antibody of claim 160.

180. A bispecific anti-PSMA x anti-CD28 antibody comprising:
a) a first monomer having an amino acid sequence of SEQ ID NO: 342;
b) a second monomer having an amino acid sequence of SEQ ID NO: 343; and
c) a light chain having an amino acid sequence of SEQ ID NO: 344.

181. A pharmaceutical composition comprising the bispecific antibody of claim 180 and a pharmaceutically acceptable carrier.

182. A nucleic acid composition comprising:
a) a first nucleic acid encoding a first monomer;
b) a second nucleic acid encoding a second monomer; and
c) a third nucleic acid encoding a light chain,
wherein the first monomer, second monomer, and light chain are the first monomer, second monomer, and light chain of claim 180, respectively.

183. An expression vector composition comprising:
a) a first expression vector comprising a first nucleic acid encoding a first monomer;
b) a second expression vector comprising a second nucleic acid encoding a second monomer; and
c) a third expression vector comprising a third nucleic acid encoding a light chain,
wherein the first monomer, second monomer and light chain are the first monomer, second monomer, and light chain of claim 180, respectively.

184. A host cell comprising the expression vector composition of claim 183.

185. A method of making a bispecific antibody comprising culturing the host cell of claim 184 under conditions wherein the bispecific antibody is expressed and recovering the bispecific antibody.

186. A method of treating prostate cancer in a patient in need thereof, comprising administering to the patient the bispecific antibody of claim 180.

187. The method of claim 186, wherein the prostate cancer is castration-resistant prostate cancer.

188. A method of enhancing T cell proliferation in the presence of PSMA-expressing cells, comprising contacting the cells with the bispecific antibody of claim 180.

189. A method of inhibiting the growth or proliferation of PSMA-expressing cells, comprising contacting the cells with the bispecific antibody of claim 180.
